(19) United States
(12) Patent Application Publication
(10) Pub. No.: US 2019/0216093 A1
(43) Pub. Date: Jul. 18, 2019

(54) COMPOSITIONS AND RELATED METHODS FOR AGRICULTURE

(71) Applicant: Flagship Pioneering Innovations V, Inc., Cambridge, MA (US)

(72) Inventors: Ignacio MARTINEZ, Lexington, MA (US); Zachary Garo ARMEN, Boston, MA (US); Barry Andrew MARTIN, Boston, MA (US); Maier Steve AVENDANO AMADO, Cambridge, MA (US)

(21) Appl. No.: 16/372,822

(22) Filed: Apr. 2, 2019

Related U.S. Application Data
(63) Continuation of application No. PCT/US2018/015077, filed on Jan. 24, 2018.
(60) Provisional application No. 62/583,763, filed on Nov. 9, 2017.
(60) Provisional application No. 62/450,045, filed on Jan. 24, 2017.

(51) Int. Cl. A01N 63/02; A01N 63/00

(52) U.S. Cl. CPC A01N 63/02; A01N 63/00

(57) ABSTRACT

Provided herein are agents, compositions, and methods for agricultural pest control, e.g., for altering the level, activity, or metabolism of one or more microorganisms resident in a host insect (e.g., agricultural pest), the alteration resulting in a decrease in the fitness of the host. The invention features a composition including an agent (e.g., phage, peptide, small molecule, antibiotic, or combinations thereof) that can alter the host's microbiota in a manner that is detrimental to the host. By disrupting microbial levels, microbial activity, microbial metabolism, and/or microbial diversity, the agents described herein may decrease the fitness of a variety of insects that are considered agricultural pests.

### CROSS-REFERENCE TO RELATED APPLICATIONS

This application claims priority to U.S. Provisional Application No. 62/450,045, filed on Jan. 24, 2017, and U.S. Provisional Application No. 62/583,763, filed on Nov. 9, 2017, the contents of which are hereby incorporated herein by reference in their entireties.

### BACKGROUND

Arthropod insects are pervasive in the human environment, and a multitude of means have been utilized for attempting to control infestations by these pests. The demand for pest control strategies is increasing. Thus, there is need in the art for new methods and compositions to control agricultural insect pests.

### SUMMARY OF THE INVENTION

Disclosed herein are compositions and methods for modulating the fitness of insects for agriculture. The composition includes an agent that alters a level, activity, or metabolism of one or more microorganisms resident in a host, the alteration resulting in a modulation in the host's fitness.

In one aspect, provided herein is a method of decreasing fitness of an agricultural insect pest, the method including delivering an antimicrobial peptide having at least 90% sequence identity (e.g., at least 90%, 92%, 94%, 96%, 98%, or 100% sequence identity) with one or more of the following: cecropin (SEQ ID NO: 82), melittin, copsin, drosomycin (SEQ ID NO: 93), dermcidin (SEQ ID NO: 81), andropin (SEQ ID NO: 83), moricin (SEQ ID NO: 84), ceratotoxin (SEQ ID NO: 85), abaecin (SEQ ID NO: 86), apidaecin (SEQ ID NO: 87), prophenin (SEQ ID NO: 88), indolicidin (SEQ ID NO: 89), protegrin (SEQ ID NO: 90), tachyplesin (SEQ ID NO: 91), or defensin (SEQ ID NO: 92) to the agricultural insect pest.

In some embodiments, the delivery may include delivering the antimicrobial peptide to at least one habitat where the agricultural insect pest grows, lives, reproduces, feeds, or infests.

In any of the above embodiments, the delivery may include spraying the antimicrobial peptide on an agricultural crop.

In any of the above embodiments, the antimicrobial peptide may be delivered as an insect comestible composition for ingestion by the agricultural insect pest.

In any of the above embodiments, the antimicrobial peptide may be formulated with an agriculturally acceptable carrier as a liquid, a solid, an aerosol, a paste, a gel, or a gas composition.

In any of the above embodiments, the agricultural insect pest may be an aphid.

In another aspect, provided herein is a composition including an antimicrobial peptide having at least 90% sequence identity (e.g., at least 90%, 92%, 94%, 96%, 98%, or 100% sequence identity) with one or more of the following: cecropin (SEQ ID NO: 82), melittin, copsin, drosomycin (SEQ ID NO: 93), dermcidin (SEQ ID NO: 81), andropin (SEQ ID NO: 83), moricin (SEQ ID NO: 84), ceratotoxin (SEQ ID NO: 85), abaecin (SEQ ID NO: 86), apidaecin (SEQ ID NO: 87), prophenin (SEQ ID NO: 88), indolicidin (SEQ ID NO: 89), protegrin (SEQ ID NO: 90), tachyplesin (SEQ ID NO: 91), or defensin (SEQ ID NO: 92) formulated for targeting a microorganism in an insect.

In some embodiments of the second aspect, the antimicrobial peptide may be at a concentration of about 0.1 ng/g to about 100 mg/g (about 0.1 ng/g to about 1 ng/g, about 1 ng/g to about 10 ng/g, about 10 ng/g to about 100 ng/g, about 100 ng/g to about 1000 ng/g, about 1 mg/g to about 10 mg/g, about 10 mg/g to about 100 mg/g) or about 0.1 ng/mL to about 100 mg/mL (about 0.1 ng/mL to about 1 ng/mL, about 1 ng/mL to about 10 ng/mL, about 10 ng/mL to about 100 ng/mL, about 100 ng/mL to about 1000 ng/mL, about 1 mg/mL to about 10 mg/mL, about 10 mg/mL to about 100 mg/mL) in the composition.

In some embodiments of the second aspect, the antimicrobial peptide may further include a targeting domain.

In some embodiments of the second aspect, the antimicrobial peptide may further include a cell penetrating peptide.

In another aspect, the composition includes an agent that alters a level, activity, or metabolism of one or more microorganisms resident in an insect host, the alteration resulting in a decrease in the insect host's fitness.

In some embodiments of any of the above compositions, the one or more microorganisms may be a bacterium or fungus resident in the host. In some embodiments, the bacterium resident in the host is at least one selected from the group consisting of Candidatus spp, Buchenera spp, Blattabacterium spp, Baumania spp, Wigglesworthia spp, Wolbachia spp, Rickettsia spp, Orientia spp, Sodalis spp, Burkholderia spp, Cupriavidus spp, Frankia spp, Snirhizobium spp, Streptococcus spp, Wolinella spp, Xylella spp, Erwinia spp, Agrobacterium spp, Bacillus spp, Paenibacillus spp, Streptomyces spp, Micrococcus spp, Corynebacterium spp, Acetobacter spp, Cyanobacteria spp, Salmonella spp, Rhodococcus spp, Pseudomonas spp, Lactobacillus spp, Enterococcus spp, Alcaligenes spp, Klebsiella spp, Paenibacillus spp, Arthrobacter spp, Corynebacterium spp, Brevibacterium spp, Thermus spp, Pseudomonas spp, Clostridium spp, and Escherichia spp. In some embodiments, the fungus resident in the host is at least one selected from the group consisting of Candida, Metschnikowia, Debaromyces, Starmerella, Pichia, Cryptococcus, Pseudozyma, Symbiotaphrina bucneri, Symbiotaphrina kochii, Scheffersomyces shehatae, Scheffersomyces stipites, Cryptococcus, Trichosporon, Amylostereum areolatum, Epichloe spp, Pichia pinus, Hansenula capsulate, Daldinia decipien, Ceratocytis spp, Ophiostoma spp, and Attamyces bromatificus. In certain embodiments, the bacteria is a Buchnera spp., (e.g., Buchnera aphidcola, an endosymbiont of aphids).

In any of the above compositions, the agent, which hereinafter may also be referred to as a modulating agent, may alter the growth, division, viability, metabolism, and/or longevity of the microorganism resident in the host. In any of the above embodiments, the modulating agent may decrease the viability of the one or more microorganisms resident in the host. In some embodiments, the modulating agent increases growth or viability of the one or more microorganisms resident in the host.

In any of the above embodiments, the modulating agent is a phage, a polypeptide, a small molecule, an antibiotic, a bacterium, or any combination thereof.

In some embodiments, the phage binds a cell surface protein on a bacterium resident in the host. In some embodiments, the phage is virulent to a bacterium resident in the host. In some embodiments, the phage is at least one selected from the group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Gluboloviridae, Guttaviridae, Inoviridae, Leviviridae, Microviridae, Plasmaviridae, and Tectiviridae.

In some embodiments, the polypeptide is at least one of a bacteriocin, R-type bacteriocin, nodule C-rich peptide, antimicrobial peptide, lysin, or bacteriocyte regulatory peptide.

In some embodiments, the small molecule is a metabolite.

In some embodiments, the antibiotic is a broad-spectrum antibiotic. In alternative embodiments, the antibiotic is a narrow-spectrum antibiotic (e.g., rifampicin).

In some embodiments, the modulating agent is a naturally occurring bacteria. In some embodiments, the bacteria is at least one selected from the group consisting of Bartonella apis, Parasaccharibacter apium, Frischella perrara, Snodgrassella alvi, Gilliamela apicola, Bifidobacterium spp, and Lactobacillus spp. In some embodiments, the bacterium is at least one selected from the group consisting of Candidatus spp, Buchenera spp, Blattabacterium spp, Baumania spp, Wigglesworthia spp, Wolbachia spp, Rickettsia spp, Orientia spp, Sodalis spp, Burkholderia spp, Cupriavidus spp, Frankia spp, Snirhizobium spp, Streptococcus spp, Wolinella spp, Xylella spp, Erwinia spp, Agrobacterium spp, Bacillus spp, Paenibacillus spp, Streptomyces spp, Micrococcus spp, Corynebacterium spp, Acetobacter spp, Cyanobacteria spp, Salmonella spp, Rhodococcus spp, Pseudomonas spp, Lactobacillus spp, Enterococcus spp, Alcaligenes spp, Klebsiella spp, Paenibacillus spp, Arthrobacter spp, Corynebacterium spp, Brevibacterium spp, Thermus spp, Pseudomonas spp, Clostridium spp, and Escherichia spp.

In any of the above compositions, host fitness may be measured by survival, reproduction, or metabolism of the host. In any of the above embodiments, the modulating agent may modulate the host's fitness by increasing pesticidal susceptibility of the host (e.g., susceptibility to a pesticide listed in Table 12). In some embodiments, the modulating agent modulates the host's fitness by increasing pesticidal susceptibility of the host. In some embodiments, the pesticidal susceptibility is bactericidal or fungicidal susceptibility. In some embodiments, the pesticidal susceptibility is insecticidal susceptibility.

In any of the above compositions, the composition may include a plurality of different modulating agents. In some embodiments, the composition includes a modulating agent and a pesticidal agent (e.g., a pesticide listed in Table 12). In some embodiments, the pesticidal agent is a bactericidal or fungicidal agent. In some embodiments, the pesticidal agent is an insecticidal agent.

In any of the above compositions, the composition may include a modulating agent and an agent that increases crop growth.

In any of the above compositions, modulating agent may be linked to a second moiety. In some embodiments, the second moiety is a modulating agent.

In any of the above compositions, the modulating agent may be linked to a targeting domain. In some embodiments, the targeting domain targets the modulating agent to a target site in the host. In some embodiments, the targeting domain targets the modulating agent to the one or more microorganisms resident in the host.

In any of the above compositions, the modulating agent may include an inactivating pre- or pro-sequence, thereby forming a precursor modulating agent. In some embodiments, the precursor modulating agent is converted to an active form in the host.

In any of the above compositions, the modulating agent may include a linker. In some embodiments, the linker is a cleavable linker.

In any of the above compositions, the composition may further include a carrier. In some instances, the carrier may be an agriculturally acceptable carrier.

In any of the above compositions, the composition may further include a host bait, a sticky agent, or a combination thereof. In some embodiments, the host bait is a comestible agent and/or a chemoattractant.

In any of the above compositions, the composition may be at a dose effective to modulate host fitness.

In any of the above compositions, the composition may be formulated for delivery to a microorganism inhabiting the gut of the host.

In any of the above compositions, the composition may be formulated for delivery to a microorganism inhabiting a bacteriocyte of the host and/or the gut of the host. In some embodiments, the composition may be formulated for delivery to a plant. In some embodiments, the composition may be formulated for use in a host feeding station.

In any of the above compositions, the composition may be formulated as a liquid, a powder, granules, or nanoparticles. In some embodiments, the composition is formulated as one selected from the group consisting of a liposome, polymer, bacteria secreting peptide, and synthetic nanocapsule. In some embodiments, the synthetic nanocapsule delivers the composition to a target site in the host. In some embodiments, the target site is the gut of the host. In some embodiments, the target site is a bacteriocyte in the host.

In yet another aspect, also provided herein are hosts that include any of the above compositions. In some embodiments, the host is an insect. In some embodiments, the insect is a species belonging to Coleoptera Diptera, Hemiptera, Lepidoptera, Orthoptera, Thysanoptera, or Acarina. In some embodiments, the insect is a beetle, weevil, fly, aphid, whitefly, leafhopper, scale, moth, butterfly, grasshopper, cricket, thrip, or mite. In certain embodiments, the insect is an aphid.

In a further aspect, also provided herein is a system for modulating a host's fitness comprising a modulating agent that targets a microorganism that is required for a host's fitness, wherein the system is effective to modulate the host's fitness, and wherein the host is an insect. The modulating agent may include any of the compositions described herein. In some embodiments, the modulating agent is formulated as a powder. In some embodiments, the modulating agent is formulated as a solvent. In some embodiments, the modulating agent is formulated as a concentrate. In some embodiments, the modulating agent is formulated as a diluent. In some embodiments, the modulating agent is prepared for delivery by combining any of the previous compositions with a carrier.

In yet a further aspect, also provided herein are methods for modulating the fitness of an insect using any of the compositions described herein. In one instance, the method of modulating the fitness of an insect host includes delivering the composition of any one of the previous claims to the host, wherein the modulating agent targets the one or more microorganisms resident in the host, and thereby modulates the host's fitness. In another instance, the method of modulating microbial diversity in an insect host includes delivering the composition of any one of the previous claims to the host, wherein the modulating agent targets the one or more microorganisms resident in the host, and thereby modulates microbial diversity in the host.

In some embodiments of any of the above methods, the modulating agent may alter the levels of the one or more microorganisms resident in the host. In some embodiments of any of the above methods, the modulating agent may alter the function of the one or more microorganisms resident in the host. In some embodiments, the one or more microorganisms may be a bacterium and/or fungus. In some embodiments, the one or more microorganisms are required for host fitness. In some embodiments, the one or more microorganisms are required for host survival.

In some embodiments of any of the above methods, the delivering step may include providing the modulating agent at a dose and time sufficient to effect the one or more microorganisms, thereby modulating microbial diversity in the host. In some embodiments, the delivering step includes topical application of any of the previous compositions to a plant. In some embodiments, the delivering step includes providing the modulating agent through a genetically engineered plant. In some embodiments, the delivering step includes providing the modulating agent to the host as a comestible. In some embodiments, the delivering step includes providing a host carrying the modulating agent. In some embodiments the host carrying the modulating agent can transmit the modulating agent to one or more additional hosts.

In some embodiments of any of the above methods, the composition may be effective to increase the host's sensitivity to a pesticidal agent (e.g., a pesticide listed in Table 12). In some embodiments, the host is resistant to the pesticidal agent prior to delivery of the modulating agent. In some embodiments, the pesticidal agent is an allelochemical agent. In some embodiments, the allelochemical agent is caffeine, soyacystatin N, monoterpenes, diterpene acids, or phenolic compounds. In some embodiments, the composition is effective to selectively kill the host. In some embodiments, the composition is effective to decrease host fitness. In some embodiments, the composition is effective to decrease the production of essential amino acids and/or vitamins in the host.

In some embodiments of any of the above methods, the host is an insect. In some embodiments, the insect is a species belonging to Coleoptera Diptera, Hemiptera, Lepidoptera, Orthoptera, Thysanoptera, or Acarina. In some embodiments, the insect is a beetle, weevil, fly, aphid, whitefly, leafhopper, scale, moth, butterfly, grasshopper, cricket, thrip, or mite. In certain embodiments, the insect is an aphid.

In some embodiments of any of the above methods, the delivering step includes delivering any of the previous compositions to a plant. In some embodiments, the plant is an agricultural crop. In some embodiments, the crop is an unharvested crop at the time of delivery. In some embodiments, the crop is a harvested crop at the time of delivery. The some embodiments, the crop comprises harvested fruits or vegetables. In some embodiments, the composition is delivered in an amount and for a duration effective to increase growth of the crop. In some embodiments, the crop includes corn, soybean, or wheat plants.

In a further aspect, also provided herein are screening assays to identify modulating agent that modulate the fitness of a host. In one instance, the screening assay to identify a modulating agent that modulates the fitness of a host, includes the steps of (a) exposing a microorganism that can be resident in the host to one or more candidate modulating agents and (b) identifying a modulating agent that decreases the fitness of the host.

In some embodiments of the screening assay, the modulating agent is a microorganism resident in the host. In some embodiments, the microorganism is a bacterium. In some embodiments, the bacterium, when resident in the host, decreases host fitness. In some embodiments of the screening assay, the modulating agent affects an allelochemical-degrading microorganism. In some embodiments, the modulating agent is a phage, an antibiotic, or a test compound. In some embodiments, the antibiotic is timentin, rifampicin, or azithromycin.

In some embodiments of the screening assay, the host may be an invertebrate. In some embodiments, the invertebrate is an insect. In some embodiments, the insect is an aphid. In some embodiments, the insect is a mosquito. In some embodiments, the insect is a cricket.

In any of the above embodiments of the screening assay, host fitness may be modulated by modulating the host microbiota.

### Definitions

As used herein, the term “bacteriocin” refers to a peptide or polypeptide that possesses anti-microbial properties. Naturally occurring bacteriocins are produced by certain prokaryotes and act against organisms related to the producer strain, but not against the producer strain itself. Bacteriocins contemplated herein include, but are not limited to, naturally occurring bacteriocins, such as bacteriocins produced by bacteria, and derivatives thereof, such as engineered bacteriocins, recombinantly expressed bacteriocins, and chemically synthesized bacteriocins. In some instances, the bacteriocin is a functionally active variant of the bacteriocins described herein. In some instances, the variant of the bacteriocin has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a bacteriocin described herein or a naturally occurring bacteriocin.

As used herein, the term “bacteriocyte” refers to a specialized cell found in certain insects where intracellular bacteria reside with symbiotic bacterial properties.

As used herein, the term “effective amount” refers to an amount of a modulating agent (e.g., a phage, lysin, bacteriocin, small molecule, or antibiotic) or composition including said agent sufficient to effect the recited result, e.g., to decrease or reduce the fitness of a host organism (e.g., insect); to reach a target level (e.g., a predetermined or threshold level) of a modulating agent concentration inside a target host; to reach a target level (e.g., a predetermined or threshold level) of a modulating agent concentration inside a target host gut; to reach a target level (e.g., a predetermined or threshold level) of a modulating agent concentration inside a target host bacteriocyte; to modulate the level, or an activity, of one or more microorganism (e.g., endosymbiont) in the target host.

As used herein, the term “fitness” refers to the ability of a host organism to survive, grow, and/or to produce surviving offspring. Fitness of an organism may be measured by one or more parameters, including, but not limited to, life span, reproductive rate, mobility, body weight, and metabolic rate. Fitness may additionally be measured based on measures of activity (e.g., biting animals or humans) or disease transmission (e.g., vector-vector transmission or vector-animal transmission).

As used herein, the term “gut” refers to any portion of a host's gut, including, the foregut, midgut, or hindgut of the host.

As used herein, the term “host” refers to an organism (e.g., insect) carrying resident microorganisms (e.g., endogenous microorganisms, endosymbiotic microorganisms (e.g., primary or secondary endosymbionts), commensal organisms, and/or pathogenic microorganisms).

As used herein “decreasing host fitness” or “decreasing host fitness” refers to any disruption to host physiology, or any activity carried out by said host, as a consequence of administration of a modulating agent, including, but not limited to, any one or more of the following desired effects: (1) decreasing a population of a host by about 10%, 20%, 30%, 40%, 50%, 60%, 70%, 80%, 90%, 95%, 99%, 100% or more; (2) decreasing the reproductive rate of a host (e.g., insect) by about 10%, 20%, 30%, 40%, 50%, 60%, 70%, 80%, 90%, 95%, 99%, 100% or more; (3) decreasing the mobility of a host (e.g., insect) by about 10%, 20%, 30%, 40%, 50%, 60%, 70%, 80%, 90%, 95%, 99%, 100% or more; (4) decreasing the body weight of a host (e.g., insect) by about 10%, 20%, 30%, 40%, 50%, 60%, 70%, 80%, 90%, 95%, 99%, 100% or more; (5) decreasing the metabolic rate or activity of a host (e.g., insect) by about 10%, 20%, 30%, 40%, 50%, 60%, 70%, 80%, 90%, 95%, 99%, 100% or more; or (6) decreasing plant infestation by a host (e.g., insect) by about 10%, 20%, 30%, 40%, 50%, 60%, 70%, 80%, 90%, 95%, 99%, 100% or more. A decrease in host fitness can be determined in comparison to a host organism to which the modulating agent has not been administered.

The term “insect” includes any organism belonging to the phylum Arthropoda and to the class Insecta or the class Arachnida, in any stage of development, i.e., immature and adult insects.

As used herein, “lysin” also known as endolysin, autolysin, murein hydrolase, peptidoglycan hydrolase, or cell wall hydrolase refers to a hydrolytic enzyme that can lyse a bacterium by cleaving peptidoglycan in the cell wall of the bacterium. Lysins contemplated herein include, but are not limited to, naturally occurring lysins, such as lysins produced by phages, lysins produced by bacteria, and derivatives thereof, such as engineered lysins, recombinantly expressed lysins, and chemically synthesized lysins. A functionally active variant of the bacteriocin may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a synthetic, recombinant, or naturally derived bacteriocin, including any described herein.

As used herein, the term “microorganism” refers to bacteria or fungi. Microorganisms may refer to microorganisms resident in a host organism (e.g., endogenous microorganisms, endosymbiotic microorganisms (e.g., primary or secondary endosymbionts)) or microorganisms exogenous to the host, including those that may act as modulating agents. As used herein, the term “target microorganism” refers to a microorganism that is resident in the host and impacted by a modulating agent, either directly or indirectly.

As used herein, the term “agent” or “modulating agent” refers to an agent that is capable of altering the levels and/or functioning of microorganisms resident in a host organism (e.g., insect), and thereby modulate (e.g., decrease) the fitness of the host organism (e.g., insect).

As used herein, the term “pesticide” or “pesticidal agent” refers to a substance that can be used in the control of agricultural, environmental, and domestic/household pests, such as insects, fungi, bacteria, and viruses. The term “pesticide” is understood to encompass naturally occurring or synthetic insecticides (larvicides or adulticides), insect growth regulators, acaricides (miticides), nematicides, ectoparasiticides, bactericides, fungicides, or herbicides (substance which can be used in agriculture to control or modify plant growth). Further examples of pesticides or pesticidal agents are listed in Table 12. In some instances, the pesticide is an allelochemical. As used herein, “allelochemical” or “allelochemical agent” is a substance produced by an organism that can effect a physiological function (e.g., the germination, growth, survival, or reproduction) of another organism (e.g., a host insect).

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “bacteriophage” or “phage” refers to a virus that infects and replicates in bacteria. Bacteriophages replicate within bacteria following the injection of their genome into the cytoplasm and do so using either a lytic cycle, which results in bacterial cell lysis, or a lysogenic (non-lytic) cycle, which leaves the bacterial cell intact. The phage may be a naturally occurring phage isolate, or an engineered phage, including vectors, or nucleic acids that encode either a partial phage genome (e.g., including at least all essential genes necessary to carry out the life cycle of the phage inside a host bacterium) or the full phage genome.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, tumor tissue, and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA, for example, of any of the modulating agents in the methods or compositions described herein.

Other features and advantages of the invention will be apparent from the following Detailed Description and the Claims.

### BRIEF DESCRIPTION OF THE DRAWINGS

The figures are meant to be illustrative of one or more features, aspects, or embodiments of the invention and are not intended to be limiting.

FIGS. 1A-1G show images of different antibiotic delivery systems. First instar LSR-1 aphids were treated with different therapeutic solutions by delivery through plants (FIG. 1A), leaf coating (FIG. 1B), microinjection (FIG. 1C), topical delivery (FIG. 1D), leaf perfusion and cutting (FIG. 1E), leaf perfusion and through plant (FIG. 1F), and combination treatment of spraying both plant and aphid, and delivery though plant (FIG. 1G).

FIG. 2A-2C show the delay in aphid development during rifampicin treatment in first instar LSR-1 aphids treated by delivery through plants with three different conditions: artificial diet without essential amino acids (AD only), artificial diet without essential amino acids with 100 μg/ml rifampicin (AD+Rif), and artificial diet with 100 μg/ml rifampicin and essential amino acids (AD+Rif+EAA). FIG. 2A is a series of graphs showing the percentage of living aphids at each developmental stage (sample size=33 aphids/group). FIG. 2B shows representative images from each treatment taken at 12 days. Scale bars 2.5 mm. FIG. 2C shows area measurements from aphid bodies showing the drastic effect of rifampicin treatment. Adding back essential amino acids partially rescues development defects.

FIG. 3 shows that rifampicin treatment resulted in aphid death. Survival was monitored daily for LSR-1 aphids treated by delivery through plants with artificial diet without essential amino acids (AD only), artificial diet without essential amino acids with 100 ug/ml rifampicin (AD+Rif), and artificial diet with 100 ug/ml rifampicin and (AD+Rif+EAA). Number in parentheses represents number of aphids in each group. Statistical significance was determined by Log-Rank Test and the following statistically significant differences were determined: AD only vs. AD+Rif, p<0.0001 and AD+Rif vs. AD+Rif+EAA, p=0.017.

FIG. 4 is a graph showing that rifampicin treatment resulted in loss of reproduction in aphids. First instar LSR-1 aphids were treated by delivery through plants with artificial diet without essential amino acids (AD only), artificial diet without essential amino acids with 100 ug/ml rifampicin (AD+Rif), and artificial diet with 100 ug/ml rifampicin and (AD+Rif+EAA) and the number of offspring produced each day after aphid reached adulthood was measured. Shown is the mean number of offspring produced per day after aphid reached adulthood±S.D.

FIG. 5 is a graph showing that rifampicin treatment eliminated endosymbiotic Buchnera. Symbiont titer was determined for the different conditions at 7 days post-treatment. DNA from aphids was extracted and qPCR was performed to determine the ratio of Buchnera DNA to aphid DNA. Shown is the mean ratio of Buchnera DNA to aphid DNA±SD of 3 aphids per group. Statistically significant differences were determined using a one-way-ANOVA followed by Tukey's Post-Test; *, p<0.05.

FIGS. 6A and 6B show that rifampicin treatment delivered through leaf coating delayed aphid development. First instar eNASCO aphids were treated by coating leaves with 100 μl of two different solutions: solvent control (0.025% Silwet L-77), and 50 μg/ml rifampicin. FIG. 6A is a series of graphs showing the developmental stage over time for each condition. Shown is the percentage of living aphids at each developmental stage (sample size=20 aphids/group). FIG. 6B is a graph showing area measurements from aphid bodies showing the drastic effect of rifampicin coated leaves on aphid size. Statistically significant differences were determined using a one-way-ANOVA followed by Tukey's Post-Test; *, p<0.05.

FIG. 7 shows that rifampicin treatment delivered through leaf coating resulted in aphid death. Survival was monitored daily for eNASCO aphids treated by coating leaves with 100 μl of two different solutions: solvent control (Silwet L-77), and 50 μg/ml rifampicin. Treatment affects survival rate of aphids.

FIG. 8 shows that rifampicin treatment delivered through leaf coating eliminated endosymbiotic Buchnera. Symbiont titer was determined for the two conditions at 6 days post-treatment. DNA from aphids was extracted and qPCR was performed to determine the ratio of Buchnera DNA to aphid DNA. Shown is the mean ratio of Buchnera DNA to aphid DNA±SD. Statistically significant differences were determined using a one-way-ANOVA followed by Tukey's Post-Test; *, p<0.05.

FIG. 9 is a graph showing rifampicin treatment by microinjection eliminated endosymbiotic Buchnera. Symbiont titer was determined 4 days post-injection with the indicated conditions. Control sample is the solvent, 0.025% Silwet L-77 described before. DNA from aphids was extracted and qPCR was performed to determine the ratio of Buchnera DNA to aphid DNA. Shown is the mean ratio of Buchnera DNA to aphid DNA±SD. Statistically significant differences were determined using a one-way-ANOVA followed by Tukey's Post-Test; *, p<0.05.

FIG. 10 is a graph showing that rifampicin treatment delivered through topical treatment eliminated endosymbiotic Buchnera. Symbiont titer was determined 3 days post-spraying with: solvent (silwet L-77) or the rifampicin solution diluted in solvent. DNA from aphids was extracted and qPCR was performed to determine the ratio of Buchnera DNA to aphid DNA. Shown is the mean ratio of Buchnera DNA to aphid DNA±SD. Statistically significant differences were determined using a one-way-ANOVA followed by Tukey's Post-Test; *, p<0.05.

FIG. 11 shows a panel of graphs demonstrating that 1^{st} and 2^{nd} instar LSR-1 aphids were placed on leaves perfused with water plus food coloring or 50 μg/ml rifampicin in water plus food coloring. Developmental stage was measured over time for each condition. Shown is the percentage of living aphids at each developmental stage (sample size=74-81 aphids/group).

FIG. 12 shows a graph demonstrating survival of 1^{st} and 2^{nd} instar LSR-1 aphids placed on leaves perfused with water plus food coloring or 50 μg/ml rifampicin in water plus food coloring. Number in parentheses represents the number of aphids in each group. Statistical significance was determined by Log-Rank Test.

FIG. 13 shows a graph demonstrating symbiont titer determined 8 days post-treatment with leaves perfused with water and food coloring or rifampicin plus water and food coloring. DNA from aphids was extracted and qPCR was performed to determine the ratio of Buchnera DNA to aphid DNA. Shown is the mean ratio of Buchnera DNA to aphid DNA±SD. Number in box indicates the median of the experimental group.

FIG. 14 shows a panel of graphs demonstrating 1^{st} and 2^{nd} instar LSR-1 aphids treated via leaf injection and through the plant with water plus food coloring or 100 μg/ml rifampicin in water plus food coloring. Developmental stage was measured over time for each condition. Shown is the percentage of living aphids at each developmental stage (sample size=49-50 aphids/group).

FIG. 15 is a graph demonstrating survival of 1^{st} and 2^{nd} instar LSR-1 aphids placed on leaves perfused and treated with water plus food coloring or 100 μg/ml rifampicin in water plus food coloring. Number in parentheses represents the number of aphids in each group. A Log-Rank Test was performed and determined that there were no statistically significant differences between groups.

FIGS. 16A and 16B are graphs showing symbiont titer determined 6 (16A) and 8 (16B) days post-treatment in aphids feeding on leaves perfused and treated with water and food coloring or rifampicin plus water and food coloring. DNA was extracted from aphids and qPCR was performed to determine the ratio of Buchnera DNA to aphid DNA. Shown is the mean ratio of Buchnera DNA to aphid DNA±SD. Number in box indicates the median of the experimental group.

FIG. 17 is a panel of graphs showing that 1^{st} and 2^{nd} instar LSR-1 aphids were treated with control solutions (water and Silwet L-77) or a combination of treatments with 100 g/ml rifampicin. Developmental stage was measured over time for each condition. Shown is the percentage of living aphids at each developmental stage (sample size=76-80 aphids/group).

FIG. 18 is a graph showing 1st and 2nd instar LSR-1 aphids were treated with control solutions of a combination of treatments containing rifampicin. Number in parentheses represents the number of aphids in each group. A Log-Rank Test was performed and determined that there were no statistically significant differences between groups.

FIG. 19 is a graph showing symbiont titer determined at 7 days post-treatment with control or rifampicin solutions. DNA from aphids was extracted and qPCR was performed to determine the ratio of Buchnera DNA to aphid DNA. Shown is the mean ratio of Buchnera DNA to aphid DNA±SD. Number in box indicates the median of the experimental group. Statistically significant differences were determined by t-test.

FIG. 20 is an image showing the chitosan delivery system. A. pisum aphids were treated with a therapeutic solution by delivery through leaf perfusion and through the plants as shown.

FIG. 21 is a panel of graphs showing that chitosan treatment resulted in delayed aphid development. First and second instar A. pisum aphids were treated by delivery through plants and leaf perfusion with the control solution (Water), and 300 ug/ml chitosan in water. Developmental stage was monitored throughout the experiment. Shown are the percent of aphids at each developmental stage (1st instar, 2nd instar, 3rd instar, 4th instar, 5th instar, or 5R which represents a reproducing 5th instar) per treatment group.

FIG. 22 is a graph showing there was a decrease in insect survival upon treatment with chitosan. First and second instar A. pisum aphids were treated by delivery through plants and leaf perfusion with just water or chitosan solution and survival was monitored daily over the course of the experiment. Number in parentheses represents the total number of aphids in the treatment group.

FIG. 23 is a graph showing treatment with chitosan reduced endosymbiotic Buchnera. First and second instar A. pisum aphids were treated by delivery through plants and leaf perfusion with water or 300 ug/ml chitosan in water. At 8 days post-treatment, DNA from aphids was extracted and qPCR was performed to determine the ratio of Buchnera DNA to aphid DNA. Shown is the mean ratio of Buchnera DNA to aphid DNA±SD of 6 aphids/group. The median value for each group is shown in box.

FIG. 24 is a panel of graphs showing treatment with nisin resulted in delayed aphid development. First and second instar LSR-2 A. pisum aphids were treated with water (control) or 1.6 or 7 mg/ml nisin via delivery by leaf injection and through the plant and development was measured over time. Shown are the percent of aphids at each life stage (1st, 2nd, 3rd, 4th, 5th, and 5R (reproducing 5th) instar) at the indicated time point. N=56-59 aphids/group.

FIG. 25 is a graph showing there was a dose dependent decrease in insect survival upon treatment with nisin. First and second instar LSR-1 A. pisum aphids were treated with water (control) or 1.6 or 7 mg/ml nisin via delivery by leaf injection and through the plant and survival was monitored over time. Number in parentheses indicates the number of aphids/group. Statistically significant differences were determined by Log Rank (Mantel-Cox) test.

FIG. 26 is a graph showing treatment with nisin reduced endosymbiotic Buchnera. First and second instar LSR-1 A. pisum aphids were treated with water (control) or 1.6 mg/ml nisin via delivery by leaf injection and through the plant and DNA was extracted from select aphids at eight days post-treatment and used for qPCR to determine Buchnera copy numbers. Shown are the mean Buchnera/aphid ratios for each treatment+/−SEM. Number in the box above each experimental group indicates the median value for that group. Each data point represents a single aphid.

FIG. 27 is a panel of graphs showing treatment with levulinic acid resulted in delayed aphid development. First and second instar eNASCO A. pisum aphids were treated with water (control) or 0.03 or 0.3% levulinic acid via delivery by leaf injection and through the plant and development was measured over time. Shown are the percent of aphids at each life stage (1^{st}, 2^{nd}, 3^{rd}, 4^{th}, and 5^{th} instar) at the indicated time point. N=57-59 aphids/group.

FIG. 28 is a graph showing there was a decrease in insect survival upon treatment with levulinic acid. First and second instar eNASCO A. pisum aphids were treated with water (control) or 0.03 or 0.3% levulinic acid via delivery by leaf injection and through the plant and survival was monitored over time. N=57-59 aphids/group. Statistically significant differences were determined by Log Rank (Mantel-Cox) test; **, p<0.01.

FIG. 29 is a panel of graphs showing treatment with levulinic acid reduced endosymbiotic Buchnera. First and second instar eNASCO A. pisum aphids were treated with water (control) or 0.03 or 0.3% levulinic acid via delivery by leaf injection and through the plant and DNA was extracted from select aphids at seven and eleven days post-treatment and used for qPCR to determine Buchnera copy numbers. Shown are the mean Buchnera/aphid ratios for each treatment+/−SEM. Statistically significant differences were determined by One-way ANOVA and Dunnett's Multiple Comparison Test; *, p<0.05. Each data point represents a single aphid.

FIGS. 30A and 30B show graphs demonstrating that gossypol treatment resulted in delayed aphid development. First and second instar A. pisum aphids were treated by delivery through plants with artificial diet without essential amino acids (AD only), and artificial diet without essential amino acids with different concentrations of gossypol (0.05%, 0.25% and 0.5%). Developmental stage was monitored throughout the experiment. FIG. 30A is a series of graphs showing the mean number of aphids at each developmental stage (1st instar, 2nd instar, 3rd instar, 4th instar, 5th instar, or 5R which represents a reproducing 5th instar) per treatment group. At the indicated time, aphids were imaged and their size was determined using Image J. FIG. 30B is a graph showing the mean aphid area±SD of artificial diet treated (Control) or gossypol treated aphids. Statistical significance was determined using a One-Way ANOVA followed by Tukey's post-test. *, p<0.05. **, p<0.01.

FIG. 31 is a graph showing a dose-dependent decrease in survival of aphids upon treatment with the allelochemical gossypol. First and second instar A. pisum aphids were treated by delivery through plants with artificial diet without essential amino acids (AD no EAA), artificial diet without essential amino acids with 0.5% gossypol acetic acid (0.5% gossypol), artificial diet without essential amino acids with 0.25% gossypol acetic acid (0.25% gossypol), and artificial diet without essential amino acids and 0.05% gossypol acetic acid (0.05% gossypol) and survival was monitored daily over the course of the experiment. Number in parentheses represents the essential amino acids number of aphids in each group. Statistically significant differences were determined by Log-Rank test and AD no EAA and 0.5% gossypol are significantly different, p=0.0002.

FIGS. 32A and 32B are two graphs showing that treatment with 0.25% gossypol resulted in decreased fecundity. First and second instar A. pisum aphids were treated by delivery through plants with artificial diet without essential amino acids (AD5-2 no EAA), or artificial diet without essential amino acids with 0.25% gossypol acetic acid (AD5-2 no EAA+0.25% gossypol), and fecundity was determined throughout the time course of the experiment. FIG. 32A shows the mean day±SD at which aphids began producing offspring was measured and gossypol treatment delayed production of offspring. FIG. 32B shows the mean number of offspring produced after the aphid began a reproducing adult±SD was measured and gossypol treatment results in decreased number of offspring produced. Each data point represents one aphid.

FIG. 33 is a graph showing that treatment with different concentrations of gossypol reduced endosymbiotic Buchnera. First and second instar A. pisum aphids were treated by delivery through plants with artificial diet without essential amino acids (Control)) or artificial diet without essential amino acids with 0.5%, 0.25%, or 0.05% gossypol. At 5 or 13 days post-treatment, DNA from aphids was extracted and qPCR was performed to determine the ratio of Buchnera DNA to aphid DNA. Shown is the mean ratio of Buchnera DNA to aphid DNA±SD of 2-6 aphids/group. Statistically significant differences were determined by Unpaired T-test; *, p<0.05.

FIG. 34 is a graph showing that microinjection of gossypol resulted in decreased Buchnera levels in aphids. A. pisum LSR-1 aphids <3rd instar stage (nymphs) were injected with 20 nl of artificial diet without essential amino acids (AD) or artificial diet without essential amino acids with 0.05% gossypol (gossypol (0.05%)). Three days after injection, DNA was extracted from aphids and Buchnera levels were assessed by qPCR. Shown are the mean ratios of Buchnera/aphid DNA±SD. Each data point represents one aphid.

FIG. 35 is a panel of graphs showing Trans-cinnemaldehyde treatment resulted in delayed aphid development. First and second instar A. pisum aphids were treated by delivery through plants with water and water with different concentrations of trans-cinnemaldehyde (TC, 0.05%, 0.5%, and 5%). Developmental stage was monitored throughout the experiment. Shown are the mean number of aphids at each developmental stage (1st instar, 2nd instar, 3rd instar, 4th instar, 5th instar, or 5R which represents a reproducing 5th instar) per treatment group. N=40-49 aphids/experimental group.

FIG. 36 is a graph showing there was a dose-dependent decrease in survival upon treatment the natural antimicrobial trans-cinnemaldehyde. First and second instar A. pisum aphids were treated by delivery through plants with water and water with different concentrations of trans-cinnemaldehyde (TC, 0.05%, 0.5%, and 5%). Survival was monitored throughout the course of the treatment. Statistically significant differences were determined by Log-Rank test. N=40-49 aphids/group.

FIG. 37 is a graph showing treatment with different concentrations of trans-cinnemaldehyde reduced endosymbiotic Buchnera. First and second instar A. pisum aphids were treated by delivery through plants with water and water with different concentrations of trans-cinnemaldehyde (0.05%, 0.5%, and 5%). At 3 days post-treatment, DNA from aphids was extracted and qPCR was performed to determine the ratio of Buchnera DNA to aphid DNA. Shown is the mean ratio of Buchnera DNA to aphid DNA±SD of 2-11 aphids/group. The median of each treatment group is shown in the box above the data points. Statistically significant differences were determined by Unpaired T-test; *, p<0.05. There was a statistically significant difference between the water control and the 0.5% trans-cinnemaldehyde group.

FIG. 38 is a panel of graphs showing treatment with scorpion peptide Uy192 resulted in delayed aphid development. First and second instar A. pisum aphids were treated by delivery through plants and leaf perfusion with the control solution (water), and 100 ug/ml Uy192 in water. a) developmental stage was monitored throughout the experiment. Shown are the percent of aphids at each developmental stage (1st instar, 2nd instar, 3rd instar, 4th instar, 5th instar, or 5R which represents a reproducing 5th instar) per treatment group.

FIG. 39 is a graph showing there was a decrease in insect survival upon treatment with the scorpion AMP Uy192. First and second instar A. pisum aphids were treated by delivery through plants and leaf perfusion with just water or Uy192 solution and survival was monitored daily over the course of the experiment. Number in parentheses represents the total number of aphids in the treatment group.

FIG. 40 is a graph showing treatment with Uy192 reduced endosymbiotic Buchnera. First and second instar A. pisum aphids were treated by delivery through plants and leaf perfusion with water or 100 ug/ml Uy192 in water, at 8 days post-treatment, DNA from aphids was extracted and qPCR was performed to determine the ratio of Buchnera DNA to aphid DNA. Shown is the mean ratio of Buchnera DNA to aphid DNA±SD of 2-6 aphids/group. The median value for each group is shown in box.

FIG. 41 is a graph showing a decrease in survival in aphids microinjected with scorpion peptides D10 and D3. LSR-1 A. pisum aphids were microinjected with water (control) or with 100 ng of either scorpion peptide D3 or D10. After injection, aphids were released to fava bean leaves and survival was monitored throughout the course of the experiment. The number in parentheses indicates the number of aphids in each experimental treatment group.

FIG. 42 is a graph showing a decrease in endosymbiont titers upon injection with scorpion peptides D3 and D10. LSR-1 A. pisum aphids were microinjected with water (control) or with 100 ng of either scorpion peptide D3 or D10. After injection, aphids were released to fava bean leaves and at 5 days post-treatment, DNA was extracted from the remaining living aphids and qPCR was performed to determine the ratio of Buchnera/aphid DNA. Shown are the mean±SD of each treatment group. N=2-9 aphids/group. The number above each treatment group in the box represents the median of the dataset.

FIG. 43 is a graph showing a decrease in insect survival upon treatment with a cocktail of scorpion AMPs. First and second instar eNASCO aphids were treated by delivery through leaf perfusion and through plants with a cocktail of scorpion peptides (40 μg/ml of each of Uy17, D3, UyCt3, and D10) and survival was monitored over the course of the experiment. The number in parentheses represents the number of aphids in each treatment group.

FIG. 44 is a panel of graphs showing treatment with scorpion peptide fused to a cell penetrating peptide resulted in delayed aphid development. First instar LSR-2 A. pisum aphids were treated with water (control) or 100 μg/ml Uy192+CPP+FAM via delivery by leaf injection and through the plant and development was measured over time. Shown are the percent of aphids at each life stage (1st, 2nd, 3rd, 4th, 5th, and 5R (reproducing 5th) instar) at the indicated time point. N=90 aphids/group.

FIG. 45 is a graph showing treatment of aphids with a scorpion peptide fused to a cell penetrating peptide increased mortality. First instar LSR-1 A. pisum aphids were treated with water or 100 μg/ml UY192+CPP+FAM (peptide) in water delivered by leaf injection and through the plant. Survival was monitored over time. The number in parentheses indicates the number of aphids/group. Statistically significant differences were determined by Log Rank (Mantel-Cox) test and there is a significant difference between the two experimental groups (p=0.0036).

FIG. 46 is a graph showing treatment with Uy192+CPP+FAM reduced endosymbiotic Buchnera. First instar LSR-1 A. pisum aphids were treated with water or 100 μg/ml Uy192+CPP+FAM (peptide) in water delivered by leaf injection and through the plant. DNA was extracted from select aphids at five days post-treatment and used for qPCR to determine Buchnera copy numbers. Shown are the mean Buchnera/aphid ratios for each treatment+/−SEM. Number in the box above each experimental group indicates the median value for that group. Each data point represents a single aphid. Statistically significant differences were determined by Student's T-test; ****, p<0.0001.

FIG. 47 is a panel of images showing Uy192+CPP+FAM penetrated bacteriocyte membranes. Bacteriocytes were dissected from the aphids and incubated with 250 ug/ml of the Uy192+CPP+FAM peptide for 30 min. Upon washing and imaging, the Uy192+CPP+FAM can be seen at high quantities inside the bacteriocytes.

FIG. 48A and FIG. 48B are a panel of graphs showing Pantothenol treatment delayed aphid development. First instar and second eNASCO aphids were treated by delivery through plants with three different conditions: artificial diet without essential amino acids (AD no EAA), artificial diet without essential amino acids with 10 uM pantothenol (10 uM pantothenol), and artificial diet without essential amino acids with 100 uM pantothenol (100 uM pantothenol), artificial diet without essential amino acids with 100 uM pantothenol, and artificial diet without essential amino acids with 10 uM pantothenol. FIG. 48A shows developmental stage monitored over time for each condition. FIG. 48B shows relative area measurements from aphid bodies showing the drastic effect of pantothenol treatment.

FIG. 49 is a graph showing that treatment with pantothenol increased aphid mortality. Survival was monitored daily for eNASCO aphids treated by delivery through plants with artificial diet without essential amino acids, or artificial diet without essential amino acids containing 10 or 100 uM pantothenol. Number in parentheses represents number of aphids in each group.

FIGS. 50A, 50B, and 50C are a panel of graphs showing Pantothenol treatment resulted in loss of reproduction. First and second instar eNASCO aphids were treated by delivery through plants with artificial diet without essential amino acids or with artificial diet without essential amino acids with 10 or 100 uM pantothenol. FIG. 50A shows the fraction of aphids surviving to maturity and reproducing. FIG. 50B shows the mean day aphids in each group began reproducing. Shown is the mean day an aphid began reproducing±SD. FIG. 50C shows the mean number of offspring produced per day after an aphid began reproducing. Shown are the mean number of offspring/day±SD.

FIG. 51 is a graph showing Pantothenol treatment did not affect endosymbiotic Buchnera. Symbiont titer was determined for the different conditions at 8 days post-treatment. DNA from aphids was extracted and qPCR was performed to determine the ratio of Buchnera DNA to aphid DNA. Shown is the mean ratio of Buchnera DNA to aphid DNA±SD of 6 aphids per group.

FIG. 52 is a panel of graphs showing Pantothenol treatment delivered through plants did not affect aphid development. First instar eNASCO aphids were treated by coating leaves with 100 μl of two different solutions: solvent control (0.025% Silwet L-77), and 10 uM pantothenol and the developmental stage was measured over time for each condition. Shown is the percentage of living aphids at each developmental stage (sample size=20 aphids/group).

FIG. 53 is a graph showing Pantothenol treatment delivered through leaf coating resulted in aphid death. Survival was monitored daily for eNASCO aphids treated by coating leaves with 100 μl of two different solutions: solvent control (Silwet L-77), and 10 uM pantothenol. Treatment affects survival rate of aphids. Sample size=20 aphids/group. Log-Rank Mantel Cox test was used to determine whether there were statistically significant differences between groups and identified that the two group are significantly different (p=0.0019).

FIGS. 54A and 54B are a panel of graphs showing treatment with a cocktail of amino acid analogs delayed aphid development. First instar LSR-1 aphids were treated by delivery through leaf perfusion and through plants with water or a cocktail of amino acid analogs in water (AA cocktail). FIG. 54A shows the developmental stage measured over time for each condition. Shown are the percentage of living aphids at each developmental stage. FIG. 54B shows the area measurements from aphid bodies showing the drastic effect of treatment with an amino acid analog cocktail (AA cocktail). Statistically significant differences were determined using a Student's T-test; ****, p<0.0001.

FIG. 55 is a graph showing treatment with a cocktail of amino acid analogs eliminated endosymbiotic Buchnera. Symbiont titer was determined for the different conditions at 6 days post-treatment. DNA from aphids was extracted and qPCR was performed to determine the ratio of Buchnera DNA to aphid DNA. Shown are the mean ratios of Buchnera DNA to aphid DNA±SD of 19-20 aphids per group. Each data point represents an individual aphid. Statistically significant differences were determined using a Student's T-test; *, p<0.05.

FIGS. 56A and 56B is a panel of graphs showing treatment with a combination of three agents delayed aphid development. First instar LSR-1 aphids were treated by delivery through leaf perfusion and through plants with water or a combination of three agents in water (Pep-Rif-Chitosan). FIG. 56A shows the developmental stage measured over time for each condition. Shown are the percentage of living aphids at each developmental stage. FIG. 56B shows the area measurements from aphid bodies showing the drastic effect of treatment with a combination of three treatments (Pep-Rif-Chitosan). Statistically significant differences were determined using a Student's T-test; ****, p<0.0001.

FIG. 57 is a graph showing treatment with a combination of a peptide, antibiotic, and natural antimicrobial agent increased aphid mortality. LSR-1 aphids were treated with water or a combination of three treatments (Pep-Rif-Chitosan) and survival was monitored daily after treatment.

FIG. 58 is a graph showing treatment with a combination of a peptide, antibiotic, and natural antimicrobial agent eliminated endosymbiotic Buchnera. Symbiont titer was determined for the different conditions at 6 days post-treatment. DNA from aphids was extracted and qPCR was performed to determine the ratio of Buchnera DNA to aphid DNA. Shown are the mean ratios of Buchnera DNA to aphid DNA±SD of 20-21 aphids per group. Each data point represents an individual aphid.

FIGS. 59A and 59B are a panel of images showing ciprofloxacin coated and penetrated corn kernels. Corn kernels were soaked in water (no antibiotic) or the indicated concentration of ciprofloxacin in water and whole kernels or kernel were tested to see whether they can inhibit the growth of E. coli DH5α. FIG. 59A shows bacterial growth in the presence of a corn kernel soaked in water without antibiotics and FIG. 59B shows the inhibition of bacterial growth when whole or half corn kernels that have been soaked in antibiotics are placed on a plate spread with E. coli.

FIG. 60 is a graph showing that adult S. zeamais weevils were treated with ciprofloxacin (250 ug/ml or 2.5 mg/ml) or mock treated with water. After 18 days of treatment, genomic DNA was isolated from weevils and the amount of Sitophilus primary endosymbiont was determined by qPCR. Shown is the mean±SEM of each group. Each data point represents one weevil. The median of each group is listed above the dataset.

FIGS. 61A and 61B are graphs showing weevil development after treatment with ciprofloxacin. FIG. 61A shows individual corn kernels cut open 25 days after adults were removed from one replicate each of the initial corn kernels soaked/coated with water (control) or ciprofloxacin (250 ug/ml or 2.5 mg/ml) and examined for the presence of larvae, pupae, or almost fully developed (adult) weevils. Shown is the percent of each life stage found in kernels from each treatment group. The total number of offspring found in the kernels from each treatment group is indicated above each dataset. FIG. 61B shows genomic DNA isolated from offspring dissected from corn kernels from the control (water) and 2.5 mg/ml ciprofloxacin treatment groups and qPCR was done to measure the amount of Sitophilus primary endosymbiont present. Shown are the mean±SD for each group. Statistically significant differences were determined by unpaired t-test; ***, p≤0.001.

FIGS. 62A and 62B are graphs showing the two remaining replicates of corn kernels mock treated (water) or treated with 250 ug/ml or 2.5 mg/ml ciprofloxacin monitored for the emergence of offspring after mating pairs were removed (at 7 days post-treatment). FIG. 62A shows the mean number of newly emerged weevils over time±SD for each treatment group. FIG. 62B shows the mean number±SEM of emerged weevils for each treatment group at 43 days after mating pairs were removed.

FIG. 63 is a panel of graphs showing rifampicin and doxycycline treatment resulted in mite mortality. Survival was monitored daily for untreated two-spotted spider mites and mites treated with 250 μg/ml rifampicin and 500 μg/ml doxycycline in 0.025% Silwet L-77.

FIG. 64 is a a panel of graphs showing results of a Seahorse flux assay for bacterial respiration. Bacteria were grown to logarithmic phase and loaded into Seahorse XFe96 plates for temporal measurements of oxygen consumption rate (OCR) and extracellular acidification rate (ECAR) as described in methods. Treatments were injected into the wells after approximately 20 minutes and bacteria were monitored to detect changes in growth. Rifampicin=100 μg/mL; Chloramphenicol=25 μg/mL; Phages (T7 for E. coli and ϕSmVL-C1 for S. marcescens) were lysates diluted either 1:2 or 1:100 in SM Buffer. The markers on each line are solely provided as indicators of the condition to which each line corresponds, and are not indicative of data points

FIG. 65 is a graph showing phage against S. marcescens reduced fly mortality. Flies that were pricked with S. marcescens were all dead within a day, whereas a sizeable portion of the flies that were pricked with both S. marcescens and the phage survived for five days after the treatment. Almost all of the control flies which were not treated in anyway survived till the end of the experiment. Log-rank test was used to compare the curves for statistical significance, asterisk denotes p<0.0001.

### DETAILED DESCRIPTION

Provided herein are methods and compositions for agricultural pest control, e.g., for altering a level, activity, or metabolism of one or more microorganisms resident in a host insect (e.g., agricultural pest), the alteration resulting in a decrease in the fitness of the host. The invention features a composition including a modulating agent (e.g., phage, peptide, small molecule, antibiotic, or combinations thereof) that can alter the host's microbiota in a manner that is detrimental to the host. By disrupting microbial levels, microbial activity, microbial metabolism, and/or microbial diversity, the modulating agent described herein may decrease the fitness of a variety of insects that are considered agricultural pests.

The methods and compositions described herein are based in part on the examples which illustrate how different agents, for example isolated natural phages, antibiotics (e.g., rifampicin, oxytetracycline, ciprofloxacin, doxycycline, or a combination thereof), antimicrobial peptides (AMPs, e.g., polymyxin B, melittin, cecropin A, drosocin, or scorpion peptides), allelochemicals (e.g., gossypol acetic acid), or natural antimicrobial compounds (e.g., trans-cinnemaldehyde, chitosan, propionic acid, levulinic acid, or nisin) can be used to target symbiotic microorganisms in insect hosts (e.g., endosymbiotic Buchnera in aphids) to decrease the fitness of these hosts by altering the level, activity, or metabolism of the microorganisms within these hosts. Rifampicin, oxytetracycline, ciprofloxacin, and doxycycline are representative examples of antibiotics, and other antibiotics may be useful in the invention. Similarly, polymyxin B, melittin, cecropin A, drosocin, or scorpion peptides are representative examples of AMPs useful in the invention. Further, gossypol acetic acid is a representative example of small molecules useful in the invention. Additionally, trans-cinnemaldehyde, chitosan, propionic acid, levulinic acid, or nisin are representative examples of useful natural antimicrobial compounds. On this basis the present disclosure describes a variety of different approaches for the use of agents that alter a level, activity, or metabolism of one or more microorganisms resident in a host, the alteration resulting in a decrease in the host's fitness.

### I. Hosts

i. Insects

The host of any of the methods and compositions provided herein may be any organism belonging to the phylum Arthropoda that is considered a pest, e.g., an agricultural pest, including any arthropods described herein. As used herein, the term “pest” refers to insects that cause damage to plants or other organisms, or otherwise are detrimental to humans, for example, human agricultural methods or products. As used herein the term “insect” describes any insect, meaning any organism belonging to the Kingdom Animals, more specific to the Phylum Arthropoda, and to the Class Insecta or the Class Arachnida.

In some instances, the insect may belong to the following orders: Acari, Araneae, Anoplura, Coleoptera, Collembola, Dermaptera, Dictyoptera, Diplura, Diptera (e.g., spotted-wing Drosophila), Embioptera, Ephemeroptera, Grylloblatodea, Hemiptera (e.g., aphids, Greenhous whitefly), Homoptera, Hymenoptera, Isoptera, Lepidoptera, Mallophaga, Mecoptera, Neuroptera, Odonata, Orthoptera, Phasmida, Plecoptera, Protura, Psocoptera, Siphonaptera, Siphunculata, Thysanura, Strepsiptera, Thysanoptera, Trichoptera, or Zoraptera.

In some instances, the insect is from the class Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vaejovis spp., Vasates lycopersici.

In some instances, the insect is from the class Chilopoda, for example, Geophilus spp., Scutigera spp.

In some instances, the insect is from the order Collembola, for example, Onychiurus armatus.

In some instances, the insect is from the class Diplopoda, for example, Blaniulus guttulatus; from the class Insecta, e.g. from the order Blattodea, for example, Blattella asahinai, Blattella germanica, Blatta orientalis, Leucophaea maderae, Panchlora spp., Parcoblatta spp., Periplaneta spp., Supella longipalpa.

In some instances, the insect is from the order Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp. (e.g., corn rootworm), Dichocrocis spp., Dicladispa armigera, Diloboderus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Necrobia spp., Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Sitophilus spp., Sitophilus oryzae, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., Zabrus spp.;

from the order Diptera, for example, Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomyia spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp. (e.g., Musca domestica), Oestrus spp., Oscinella frit, Paratanytarsus spp., Paralauterborniella subcincta, Pegomyia spp., Phlebotomus spp., Phorbia spp., Phormia spp., Piophila casei, Prodiplosis spp., Psila rosae, Rhagoletis spp., Sarcophaga spp., Simulium spp., Stomoxys spp., Tabanus spp., Tetanops spp., Tipula spp.

In some instances, the insect is from the order Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptocorisa varicornis, Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.

In some instances, the insect is from the order Homoptera, for example, Acizzia acaciaebaileyanae, Acizzia dodonaeae, Acizzia uncatoides, Acrida turrita, Acyrthosipon spp., Acrogonia spp., Aeneolamia spp., Agonoscena spp., Aleyrodes proletella, Aleurolobus barodensis, Aleurothrixus floccosus, Allocaridara malayensis, Amrasca spp., Anuraphis cardui, Aonidiella spp., Aphanostigma pini, Aphis spp. (e.g., Apis gossypii), Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes citri, Diaphorina citri, Diaspis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Homalodisca vitripennis, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Macrosteles facifrons, Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nettigoniclla spectra, Nilaparvata lugens, Oncometopia spp., Orthezia praelonga, Oxya chinensis, Pachypsylla spp., Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.; from the order Hymenoptera, for example, Acromyrmex spp., Athalia spp., Atta spp., Diprion spp., Hoplocampa spp., Lasius spp., Monomorium pharaonis, Sirex spp., Solenopsis invicta, Tapinoma spp., Urocerus spp., Vespa spp., Xeris spp.

In some instances, the insect is from the order Isopoda, for example, Armadillidium vulgare, Oniscus asellus, Porcellio scaber.

In some instances, the insect is from the order Isoptera, for example, Coptotermes spp., Cornitermes cumulans, Cryptotermes spp., Incisitermes spp., Microtermes obesi, Odontotermes spp., Reticulitermes spp.

In some instances, the insect is from the order Lepidoptera, for example, Achroia grisella, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamstra brassicae, Melanitis leda, Mocis spp., Monopis obviella, Mythimna separata, Nemapogon cloacellus, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., Tryporyza incertulas, Tuta absoluta, Virachola spp.

In some instances, the insect is from the order Orthoptera or Saltatoria, for example, Acheta domesticus, Dichroplus spp., Gryllotalpa spp., Hieroglyphus spp., Locusta spp., Melanoplus spp., Schistocerca gregaria.

In some instances, the insect is from the order Phthiraptera, for example, Damalinia spp., Haematopinus spp., Linognathus spp., Pediculus spp., Ptirus pubis, Trichodectes spp.

In some instances, the insect is from the order Psocoptera for example Lepinatus spp., Liposcelis spp.

In some instances, the insect is from the order Siphonaptera, for example, Ceratophyllus spp., Ctenocephalides spp., Pulex irritans, Tunga penetrans, Xenopsylla cheopsis.

In some instances, the insect is from the order Thysanoptera, for example, Anaphothrips obscurus, Baliothrips biformis, Drepanothrips reuteri, Enneothrips flavens, Frankliniella spp., Heliothrips spp., Hercinothrips femoralis, Rhipiphorothrips cruentatus, Scirtothrips spp., Taeniothrips cardamomi, Thrips spp.

In some instances, the insect is from the order Zygentoma (=Thysanura), for example, Ctenolepisma spp., Lepisma saccharina, Lepismodes inquilinus, Thermobia domestica.

In some instances, the insect is from the class Symphyla, for example, Scutigerella spp.

In some instances, the insect is a mite, including but not limited to, Tarsonemid mites, such as Phytonemus pallidus, Polyphagotarsonemus latus, Tarsonemus bilobatus, or the like; Eupodid mites, such as Penthaleus erythrocephalus, Penthaleus major, or the like; Spider mites, such as Oligonychus shinkajii, Panonychus citri, Panonychus mori, Panonychus ulmi, Tetranychus kanzawai, Tetranychus urticae, or the like; Eriophyid mites, such as Acaphylla theavagrans, Aceria tulipae, Aculops lycopersici, Aculops pelekassi, Aculus schlechtendali, Eriophyes chibaensis, Phyllocoptruta oleivora, or the like; Acarid mites, such as Rhizoglyphus robini, Tyrophagus putrescentiae, Tyrophagus similis, or the like; Bee brood mites, such as Varroa jacobsoni, Varroa destructor or the like; Ixodides, such as Boophilus microplus, Rhipicephalus sanguineus, Haemaphysalis longicornis, Haemophysalis flava, Haemophysalis campanulata, Ixodes ovatus, Ixodes persulcatus, Amblyomma spp., Dermacentor spp., or the like; Cheyletidae, such as Cheyletiella yasguri, Cheyletiella blakei, or the like; Demodicidae, such as Demodex canis, Demodex cati, or the like; Psoroptidae, such as Psoroptes ovis, or the like; Scarcoptidae, such as Sarcoptes scabiei, Notoedres cati, Knemidocoptes spp., or the like.

In certain instances, the insect is an aphid. In certain instances, the insect is a weevil. In certain instances, the insect is a two-spotted spider mite. In certain instances, the insect is a fall army worm. In certain instances, the insect is a Varroa mite (e.g., a Varroa mite that infects bees).

ii. Host Fitness

The methods and compositions provided herein may be used to decrease the fitness of any of the hosts described herein. The decrease in fitness may arise from any alterations in microorganisms resident in the host, wherein the alterations are a consequence of administration of a modulating agent and have detrimental effects on the host.

In some instances, the decrease in host fitness may manifest as a deterioration or decline in the physiology of the host (e.g., reduced health or survival) as a consequence of administration of a modulating agent. In some instances, the fitness of an organism may be measured by one or more parameters, including, but not limited to, reproductive rate, lifespan, mobility, fecundity, body weight, metabolic rate or activity, or survival in comparison to a host organism to which the modulating agent has not been administered. For example, the methods or compositions provided herein may be effective to decrease the overall health of the host or to decrease the overall survival of the host. In some instances, the decreased survival of the host is about 2%, 5%, 10%, 20%, 30%, 40%, 50%, 60%, 70%, 80%, 90%, 100%, or greater than 100% greater relative to a reference level (e.g., a level found in a host that does not receive a modulating agent). In some instances, the methods and compositions are effective to decrease host reproduction (e.g., reproductive rate) in comparison to a host organism to which the modulating agent has not been administered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a host that does not receive a modulating agent).

In some instances, the decrease in host fitness may manifest as a decrease in the production of one or more nutrients in the host (e.g., vitamins, carbohydrates, amino acids, or polypeptides) in comparison to a host organism to which the modulating agent has not been administered. In some instances, the methods or compositions provided herein may be effective to decrease the production of nutrients in the host (e.g., vitamins, carbohydrates, amino acids, or polypeptides) by about 2%, 5%, 10%, 20%, 30%, 40%, 50%, 60%, 70%, 80%, 90%, 100%, or greater than 100% relative to a reference level (e.g., a level found in a host that does not receive a modulating agent). In some instances, the methods or compositions provided herein may decrease nutrients in the host by decreasing the production of nutrients by one or more microorganisms (e.g., endosymbiont) in the host in comparison to a host organism to which the modulating agent has not been administered.

In some instances, the decrease in host fitness may manifest as an increase in the host's sensitivity to a pesticidal agent (e.g., a pesticide listed in Table 12) and/or a decrease in the host's resistance to a pesticidal agent (e.g., a pesticide listed in Table 12) in comparison to a host organism to which the modulating agent has not been administered. In some instances, the methods or compositions provided herein may be effective to increase the host's sensitivity to a pesticidal agent (e.g., a pesticide listed in Table 12) by about 2%, 5%, 10%, 20%, 30%, 40%, 50%, 60%, 70%, 80%, 90%, 100%, or greater than 100% relative to a reference level (e.g., a level found in a host that does not receive a modulating agent). The pesticidal agent may be any pesticidal agent known in the art, including insecticidal agents. In some instances, the methods or compositions provided herein may increase the host's sensitivity to a pesticidal agent (e.g., a pesticide listed in Table 12) by decreasing the host's ability to metabolize or degrade the pesticidal agent into usable substrates.

In some instances, the decrease in host fitness may manifest as an increase in the host's sensitivity to an allelochemical agent and/or a decrease in the host's resistance to an allelochemical agent in comparison to a host organism to which the modulating agent has not been administered. In some instances, the methods or compositions provided herein may be effective to decrease the host's resistance to an allelochemical agent by about 2%, 5%, 10%, 20%, 30%, 40%, 50%, 60%, 70%, 80%, 90%, 100%, or greater than 100% relative to a reference level (e.g., a level found in a host that does not receive a modulating agent). In some instances, the allelochemical agent is caffeine, soyacystatin N, monoterpenes, diterpene acids, or phenolic compounds. In some instances, the methods or compositions provided herein may increase the host's sensitivity to an allelochemical agent by decreasing the host's ability to metabolize or degrade the allelochemical agent into usable substrates in comparison to a host organism to which the modulating agent has not been administered.

In some instances, the methods or compositions provided herein may be effective to decease the host's resistance to parasites or pathogens (e.g., fungal, bacterial, or viral pathogens or parasites) in comparison to a host organism to which the modulating agent has not been administered. In some instances, the methods or compositions provided herein may be effective to decrease the host's resistance to a pathogen or parasite (e.g., fungal, bacterial, or viral pathogens; or parasitic mites) by about 2%, 5%, 10%, 20%, 30%, 40%, 50%, 60%, 70%, 80%, 90%, 100%, or greater than 100% relative to a reference level (e.g., a level found in a host that does not receive a modulating agent).

In some instances, the decrease in host fitness may manifest as other fitness disadvantages, such as decreased tolerance to certain environmental factors (e.g., a high or low temperature tolerance), decreased ability to survive in certain habitats, or a decreased ability to sustain a certain diet in comparison to a host organism to which the modulating agent has not been administered. In some instances, the methods or compositions provided herein may be effective to decrease host fitness in any plurality of ways described herein. Further, the modulating agent may decrease host fitness in any number of host classes, orders, families, genera, or species (e.g., 1 host species, 2, 3, 4, 5, 6, 7, 8, 9, 10, 15, 20, 30, 40, 50, 60, 70, 80, 90, 100, 150, 200, 200, 250, 500, or more host species). In some instances, the modulating agent acts on a single host class, order, family, genus, or species.

Host fitness may be evaluated using any standard methods in the art. In some instances, host fitness may be evaluated by assessing an individual host. Alternatively, host fitness may be evaluated by assessing a host population. For example, a decrease in host fitness may manifest as a decrease in successful competition against other insects, thereby leading to a decrease in the size of the host population.

iii. Host Insects in Agriculture

By reducing the fitness of harmful insects, the modulating agents provided herein may be effective to promote the growth of plants that are typically harmed by said hosts. The modulating agent may be delivered to the plant using any of the formulations and delivery methods described herein, in an amount and for a duration effective to decrease host fitness and thereby benefit the plant, e.g., increase crop growth, increase crop yield, decrease pest infestation, and/or decrease damage to plants. This may or may not involve direct application of the modulating agent to the plant. For example, in instances where the primary host habitat is different than the region of plant growth, the modulating agent may be applied to either the primary host habitat, the plants of interest, or a combination of both.

In some instances, the plant may be an agricultural food crop, such as a cereal, grain, legume, fruit, or vegetable crop, or a non-food crop, e.g., grasses, flowering plants, cotton, hay, hemp. The compositions described herein may be delivered to the crop any time prior to or after harvesting the cereal, grain, legume, fruit, vegetable, or other crop. Crop yield is a measurement often used for crop plants and is normally measured in metric tons per hectare (or kilograms per hectare). Crop yield can also refer to the actual seed generation from the plant. In some instances, the modulating agent may be effective to increase crop yield (e.g., increase metric tons of cereal, grain, legume, fruit, or vegetable per hectare and/or increase seed generation) by about 2%, 5%, 10%, 20%, 30%, 40%, 50%, 60%, 70%, 80%, 90%, 100%, or more in comparison to a reference level (e.g., a crop to which the modulating agent has not been administered).

In some instances, the plant (e.g., crop) may be at risk of developing a pest infestation (e.g., by an insect) or may have already developed a pest infestation. The methods and compositions described herein may be used to reduce or prevent pest infestation in such crops by reducing the fitness of insects that infest the plants. In some instances, the modulating agent may be effective to reduce crop infestation (e.g., reduce the number of plants infested, reduce the pest population size, reduce damage to plants) by about 2%, 5%, 10%, 20%, 30%, 40%, 50%, 60%, 70%, 80%, 90%, 100%, or more in comparison to a reference level (e.g., a crop to which the modulating agent has not been administered). In other instances, the modulating agent may be effective to prevent or reduce the likelihood of crop infestation by about 2%, 5%, 10%, 20%, 30%, 40%, 50%, 60%, 70%, 80%, 90%, 100%, or more in comparison to a reference level (e.g., a crop to which the modulating agent has not been administered).

Any suitable plant tissues may benefit from the compositions and methods described herein, including, but not limited to, somatic embryos, pollen, leaves, stems, calli, stolons, microtubers, and shoots. The methods described herein may include treatment of angiosperm and gymnosperm plants such as acacia, alfalfa, apple, apricot, artichoke, ash tree, asparagus, avocado, banana, barley, beans, beet, birch, beech, blackberry, blueberry, broccoli, brussels sprouts, cabbage, canola, cantaloupe, carrot, cassaya, cauliflower, cedar, a cereal, celery, chestnut, cherry, Chinese cabbage, citrus, clemintine, clover, coffee, corn, cotton, conifers, cowpea, cucumber, cypress, eggplant, elm, endive, eucalyptus, fava beans, fennel, figs, fir, fruit and nut trees, geranium, grape, grapefruit, groundnuts, ground cherry, gum hemlock, hemp, hickory, kale, kiwifruit, kohlrabi, larch, lettuce, leek, lemon, lime, locust, pine, maidenhair, maize, mango, maple, melon, millet, mushroom, mustard, nuts, oak, oats, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, sallow, soybean, spinach, spruce, squash, strawberry, sugarbeet, sugarcane, sunflower, sweet potato, sweet corn, tangerine, tea, tobacco, tomato, trees, triticale, turf grasses, turnips, a vine, walnut, watercress, watermelon, wheat, yams, yew, and zucchini.

II. Target Microorganisms

The microorganisms targeted by the modulating agent described herein may include any microorganism resident in or on the host, including, but not limited to, any bacteria and/or fungi described herein. Microorganisms resident in the host may include, for example, symbiotic (e.g., endosymbiotic microorganisms that provide beneficial nutrients or enzymes to the host), commensal, pathogenic, or parasitic microorganisms. An endosymbiotic microorganism may be a primary endosymbiont or a secondary endosymbiont. A symbiotic microorganism (e.g., bacteria or fungi) may be an obligate symbiont of the host or a facultative symbiont of the host. Microorganisms resident in the host may be acquired by any mode of transmission, including vertical, horizontal, or multiple origins of transmission.

i. Bacteria

Exemplary bacteria that may be targeted in accordance with the methods and compositions provided herein, include, but are not limited to, Xenorhabdus spp, Photorhabdus spp, Candidatus spp, Buchnera spp, Blattabacterium spp, Baumania spp, Wigglesworthia spp, Wolbachia spp, Rickettsia spp, Orientia spp, Sodalis spp, Burkholderia spp, Cupriavidus spp, Frankia spp, Snirhizobium spp, Streptococcus spp, Wolinella spp, Xylella spp (e.g., Xylella fastidiosa), Erwinia spp, Agrobacterium spp, Bacillus spp, Commensalibacter spp. (e.g., Commensalibacter intestine), Paenibacillus spp, Streptomyces spp, Micrococcus spp, Corynebacterium spp, Acetobacter spp (e.g., Acetobacter pomorum), Cyanobacteria spp, Salmonella spp, Rhodococcus spp, Pseudomonas spp, Lactobacillus spp (e.g., Lactobacillus plantarum), Lysobacter spp., Herbaspirillum spp., Enterococcus spp, Gluconobacter spp. (e.g., Gluconobacter morbifer), Alcaligenes spp, Hamiltonella spp., Klebsiella spp, Paenibacillus spp, Serratia spp., Arthrobacter spp, Azotobacter spp., Corynebacterium spp, Brevibacterium spp, Regiella spp. (e.g., Regiella insecticola), Thermus spp, Pseudomonas spp, Clostridium spp, Mortierella spp. (e.g., Mortierella elongata) and Escherichia spp. In some instances, the bacteria targeted by the modulating agent may be ones that can be transmitted from the insect to a plant, including, but not limited to, bacterial plant pathogens (e.g., Agrobacterium spp.). Non-limiting examples of bacteria that may be targeted by the methods and compositions provided herein are shown in Table 1. In some instances, the 16S rRNA sequence of the bacteria targeted by the modulating agent has at least 50%, 60%, 70%, 80%, 85%, 90%, 95%, 97%, 99%, 99.9%, or 100% identity with a sequence listed in Table 1.

TABLE 1
Examples of Target Bacteria and Host Insects

| Primary endosymbiont | Host | Location | 16S rRNA |
|---|---|---|---|
| Gamma proteobacteria | | | |
| Carsonella ruddii | Psyllids | bacteriocytes | TATCCAGCCACAGGTTCCCCTA |
| | (Psylloidea) | | CAGCTACCTTGTTACGACTTCA |
| | | | CCCCAGTTACAAATCATACCGTT |
| | | | GTAATAGTAAAATTACTTATGAT |
| | | | ACAATTTACTTCCATGGTGTGAC |
| | | | GGGCGGTGTGTACAAGGCTCG |
| | | | AGAACGTATTCACCGTAACATTC |
| | | | TGATTTACGATTACTAGCGATTC |
| | | | CAACTTCATGAAATCGAGTTACA |
| | | | GATTTCAATCCGAACTAAGAATA |
| | | | TTTTTTAAGATTAGCATTATGTT |
| | | | GCCATATAGCATATAACTTTTTG |
| | | | TAATACTCATTGTAGCACGTGTG |
| | | | TAGCCCTACTTATAAGGGCCAT |
| | | | GATGACTTGACGTCGTCCTCAC |
| | | | CTTCCTCCAATTTATCATTGGCA |
| | | | GTTTCTTATTAGTTCTAATATATT |
| | | | TTTAGTAAAATAAGATAAGGGTT |
| | | | GCGCTCGTTATAGGACTTAACC |
| | | | CAACATTTCACAACACGAGCTG |
| | | | ACGACAGCCATGCAGCACCTGT |
| | | | CTCAAAGCTAAAAAAGCTTTATT |
| | | | ATTTCTAATAAATTCTTTGGATG |
| | | | TCAAAAGTAGGTAAGATTTTTCG |
| | | | TGTTGTATCGAATTAAACCACAT |
| | | | GCTCCACCGCTTGTGCGAGCCC |
| | | | CCGTCAATTCATTTGAGTTTTAA |
| | | | CCTTGCGGTCGTAATCCCCAGG |
| | | | CGGTCAACTTAACGCGTTAGCT |
| | | | TTTTCACTAAAAATATATAACTTT |
| | | | TTTTCATAAAACAAAATTACAATT |
| | | | ATAATATTTAATAAATAGTTGAC |
| | | | ATCGTTTACTGCATGGACTACC |
| | | | AGGGTATCTAATCCTGTTTGCTC |
| | | | CCCATGCTTTCGTGTATTAGTGT |
| | | | CAGTATTAAAATAGAAATACGCC |
| | | | TTCGCCACTAGTATTCTTTCAGA |
| | | | TATCTAAGCATTTCACTGCTACT |
| | | | CCTGAAATTCTAATTTCTTCTTTT |
| | | | ATACTCAAGTTTATAAGTATTAA |
| | | | TTTCAATATTAAATTACTTTAATA |
| | | | AATTTAAAAATTAATTTTTAAAAA |
| | | | CAACCTGCACACCCTTTACGCC |
| | | | CAATAATTCCGATTAACGCTTGC |
| | | | ACCCCTCGTATTACCGCGGCTG |
| | | | CTGGCACGAAGTTAGCCGGTGC |
| | | | TTCTTTTACAAATAACGTCAAAG |
| | | | ATAATATTTTTTTATTATAAAATC |
| | | | TCTTCTTACTTTGTTGAAAGTGT |
| | | | TTTACAACCCTAAGGCCTTCTTC |
| | | | ACACACGCGATATAGCTGGATC |
| | | | AAGCTTTCGCTCATTGTCCAATA |
| | | | TCCCCCACTGCTGCCTTCCGTA |
| | | | AAAGTTTGGGCCGTGTCTCAGT |
| | | | CCCAATGTGGTTGTTCATCCTCT |
| | | | AAGATCAACTACGAATCATAGTC |
| | | | TTGTTAAGCTTTTACTTTAACAA |
| | | | CTAACTAATTCGATATAAGCTCT |
| | | | TCTATTAGCGAACGACATTCTC |
| | | | GTTCTTTATCCATTAGGATACAT |
| | | | ATTGAATTACTATACATTTCTATA |
| | | | TACTTTTCTAATACTAATAGGTA |
| | | | GATTCTTATATATTACTCACCCG |
| | | | TTCGCTGCTAATTATTTTTTTAAT |
| | | | AATTCGCACAACTTGCATGTGTT |
| | | | AAGCTTATCGCTAGCGTTCAAT |
| | | | CTGAGCTATGATCAAACTCA |
| | | | (SEQ ID NO: 1) |
| Portiera aleyrodidarum | whiteflyes | bacteriocytes | AAGAGTTTGATCATGGCTCAGA |
| BT-B | (Aleyrodoidea) | | TTGAACGCTAGCGGCAGACATA |
| | | | ACACATGCAAGTCGAGCGGCAT |
| | | | CATACAGGTTGGCAAGCGGCG |
| | | | CACGGGTGAGTAATACATGTAA |
| | | | ATATACCTAAAAGTGGGGAATA |
| | | | ACGTACGGAAACGTACGCTAAT |
| | | | ACCGCATAATTATTACGAGATAA |
| | | | AGCAGGGGCTTGATAAAAAAAA |
| | | | TCAACCTTGCGCTTTTAGAAAAT |
| | | | TACATGCCGGATTAGCTAGTTG |
| | | | GTAGAGTAAAAGCCTACCAAGG |
| | | | TAACGATCCGTAGCTGGTCTGA |
| | | | GAGGATGATCAGCCACACTGGG |
| | | | ACTGAGAAAAGGCCCAGACTCC |
| | | | TACGGGAGGCAGCAGTGGGGA |
| | | | ATATTGGACAATGGGGGGAACC |
| | | | CTGATCCAGTCATGCCGCGTGT |
| | | | GTGAAGAAGGCCTTTGGGTTGT |
| | | | AAAGCACTTTCAGCGAAGAAGA |
| | | | AAAGTTAGAAAATAAAAAGTTAT |
| | | | AACTATGACGGTACTCGCAGAA |
| | | | GAAGCACCGGCTAACTCCGTGC |
| | | | CAGCAGCCGCGGTAAGACGGA |
| | | | GGGTGCAAGCGTTAATCAGAAT |
| | | | TACTGGGCGTAAAGGGCATGTA |
| | | | GGTGGTTTGTTAAGCTTTATGTG |
| | | | AAAGCCCTATGCTTAACATAGG |
| | | | AACGGAATAAAGAACTGACAAA |
| | | | CTAGAGTGCAGAAGAGGAAGGT |
| | | | AGAATTCCCGGTGTAGCGGTGA |
| | | | AATGCGTAGATATCTGGAGGAA |
| | | | TACCAGTTGCGAAGGCGACCTT |
| | | | CTGGGCTGACACTGACACTGAG |
| | | | ATGCGAAAGCGTGGGGAGCAA |
| | | | ACAGGATTAGATACCCTGGTAG |
| | | | TCCACGCTGTAAACGATATCAA |
| | | | CTAGCCGTTGGATTCTTAAAGA |
| | | | ATTTTGTGGCGTAGCTAACGCG |
| | | | ATAAGTTGATCGCCTGGGGAGT |
| | | | ACGGTCGCAAGGCTAAAACTCA |
| | | | AATGAATTGACGGGGGCCCGCA |
| | | | CAAGCGGTGGAGCATGTGGTTT |
| | | | AATTCGATGCAACGCGCAAAAC |
| | | | CTTACCTACTCTTGACATCCAAA |
| | | | GTACTTTCCAGAGATGGAAGGG |
| | | | TGCCTTAGGGAACTTTGAGACA |
| | | | GGTGCTGCATGGCTGTCGTCAG |
| | | | CTCGTGTTGTGAAATGTTGGGT |
| | | | TAAGTCCCGTAACGAGCGCAAC |
| | | | CCTTGTCCTTAGTTGCCAACGC |
| | | | ATAAGGCGGGAACTTTAAGGAG |
| | | | ACTGCTGGTGATAAACCGGAGG |
| | | | AAGGTGGGGACGACGTCAAGT |
| | | | CATCATGGCCCTTAAGAGTAGG |
| | | | GCAACACACGTGCTACAATGGC |
| | | | AAAAACAAAGGGTCGCAAAATG |
| | | | GTAACATGAAGCTAATCCCAAA |
| | | | AAAATTGTCTTAGTTCGGATTGG |
| | | | AGTCTGAAACTCGACTCCATAA |
| | | | AGTCGGAATCGCTAGTAATCGT |
| | | | GAATCAGAATGTCACGGTGAAT |
| | | | ACGTTCTCGGGCCTTGTACACA |
| | | | CCGCCCGTCACACCATGGAAGT |
| | | | GAAATGCACCAGAAGTGGCAAG |
| | | | TTTAACCAAAAAACAGGAGAAC |
| | | | AGTCACTACGGTGTGGTTCATG |
| | | | ACTGGGGTGAAGTCGTAACAAG |
| | | | GTAGCTGTAGGGGAACCTGTGG |
| | | | CTGGATCACCTCCTTAA |
| | | | (SEQ ID NO: 2) |
| Buchnera aphidicola str. | Aphids | bacteriocytes | AGAGTTTGATCATGGCTCAGAT |
| APS (Acyrthosiphon | (Aphidoidea) | | TGAACGCTGGCGGCAAGCCTAA |
| pisum) | | | CACATGCAAGTCGAGCGGCAG |
| | | | CGAGAAGAGAGCTTGCTCTCTT |
| | | | TGTCGGCAAGCGGCAAACGGG |
| | | | TGAGTAATATCTGGGGATCTAC |
| | | | CCAAAAGAGGGGGATAACTACT |
| | | | AGAAATGGTAGCTAATACCGCA |
| | | | TAATGTTGAAAAACCAAAGTGG |
| | | | GGGACCTTTTGGCCTCATGCTT |
| | | | TTGGATGAACCCAGACGAGATT |
| | | | AGCTTGTTGGTAGAGTAATAGC |
| | | | CTACCAAGGCAACGATCTCTAG |
| | | | CTGGTCTGAGAGGATAACCAGC |
| | | | CACACTGGAACTGAGACACGGT |
| | | | CCAGACTCCTACGGGAGGCAG |
| | | | CAGTGGGGAATATTGCACAATG |
| | | | GGCGAAAGCCTGATGCAGCTAT |
| | | | GCCGCGTGTATGAAGAAGGCCT |
| | | | TAGGGTTGTAAAGTACTTTCAG |
| | | | CGGGGAGGAAAAAAATAAAACT |
| | | | AATAATTTTATTTCGTGACGTTA |
| | | | CCCGCAGAAGAAGCACCGGCT |
| | | | AACTCCGTGCCAGCAGCCGCG |
| | | | GTAATACGGAGGGTGCAAGCGT |
| | | | TAATCAGAATTACTGGGCGTAA |
| | | | AGAGCGCGTAGGTGGTTTTTTA |
| | | | AGTCAGGTGTGAAATCCCTAGG |
| | | | CTCAACCTAGGAACTGCATTTG |
| | | | AAACTGGAAAACTAGAGTTTCG |
| | | | TAGAGGGAGGTAGAATTCTAGG |
| | | | TGTAGCGGTGAAATGCGTAGAT |
| | | | ATCTGGAGGAATACCCGTGGCG |
| | | | AAAGCGGCCTCCTAAACGAAAA |
| | | | CTGACACTGAGGCGCGAAAGC |
| | | | GTGGGGAGCAAACAGGATTAGA |
| | | | TACCCTGGTAGTCCATGCCGTA |
| | | | AACGATGTCGACTTGGAGGTTG |
| | | | TTTCCAAGAGAAGTGACTTCCG |
| | | | AAGCTAACGCATTAAGTCGACC |
| | | | GCCTGGGGAGTACGGCCGCAA |
| | | | GGCTAAAACTCAAATGAATTGA |
| | | | CGGGGGCCCGCACAAGCGGTG |
| | | | GAGCATGTGGTTTAATTCGATG |
| | | | CAACGCGAAAAACCTTACCTGG |
| | | | TCTTGACATCCACAGAATTCTTT |
| | | | AGAAATAAAGAAGTGCCTTCGG |
| | | | GAGCTGTGAGACAGGTGCTGCA |
| | | | TGGCTGTCGTCAGCTCGTGTTG |
| | | | TGAAATGTTGGGTTAAGTCCCG |
| | | | CAACGAGCGCAACCCTTATCCC |
| | | | CTGTTGCCAGCGGTTCGGCCG |
| | | | GGAACTCAGAGGAGACTGCCG |
| | | | GTTATAAACCGGAGGAAGGTGG |
| | | | GGACGACGTCAAGTCATCATGG |
| | | | CCCTTACGACCAGGGCTACACA |
| | | | CGTGCTACAATGGTTTATACAAA |
| | | | GAGAAGCAAATCTGCAAAGACA |
| | | | AGCAAACCTCATAAAGTAAATC |
| | | | GTAGTCCGGACTGGAGTCTGCA |
| | | | ACTCGACTCCACGAAGTCGGAA |
| | | | TCGCTAGTAATCGTGGATCAGA |
| | | | ATGCCACGGTGAATACGTTCCC |
| | | | GGGCCTTGTACACACCGCCCGT |
| | | | CACACCATGGGAGTGGGTTGCA |
| | | | AAAGAAGCAGGTATCCTAACCC |
| | | | TTTAAAAGGAAGGCGCTTACCA |
| | | | CTTTGTGATTCATGACTGGGGT |
| | | | GAAGTCGTAACAAGGTAACCGT |
| | | | AGGGGAACCTGCGGTTGGATCA |
| | | | CCTCCTT |
| | | | (SEQ ID NO: 3) |
| Buchnera aphidicola str. | Aphids | bacteriocytes | AAACTGAAGAGTTTGATCATGG |
| Sg (Schizaphis | (Aphidoidea) | | CTCAGATTGAACGCTGGCGGCA |
| graminum) | | | AGCCTAACACATGCAAGTCGAG |
| | | | CGGCAGCGAAAAGAAAGCTTGC |
| | | | TTTCTTGTCGGCGAGCGGCAAA |
| | | | CGGGTGAGTAATATCTGGGGAT |
| | | | CTGCCCAAAAGAGGGGGATAAC |
| | | | TACTAGAAATGGTAGCTAATACC |
| | | | GCATAAAGTTGAAAAACCAAAG |
| | | | TGGGGGACCTTTTTTAAAGGCC |
| | | | TCATGCTTTTGGATGAACCCAG |
| | | | ACGAGATTAGCTTGTTGGTAAG |
| | | | GTAAAAGCTTACCAAGGCAACG |
| | | | ATCTCTAGCTGGTCTGAGAGGA |
| | | | TAACCAGCCACACTGGAACTGA |
| | | | GACACGGTCCAGACTCCTACGG |
| | | | GAGGCAGCAGTGGGGAATATTG |
| | | | CACAATGGGCGAAAGCCTGATG |
| | | | CAGCTATGCCGCGTGTATGAAG |
| | | | AAGGCCTTAGGGTTGTAAAGTA |
| | | | CTTTCAGCGGGGAGGAAAAAAT |
| | | | TAAAACTAATAATTTTATTTTGTG |
| | | | ACGTTACCCGCAGAAGAAGCAC |
| | | | CGGCTAACTCCGTGCCAGCAGC |
| | | | CGCGGTAATACGGAGGGTGCG |
| | | | AGCGTTAATCAGAATTACTGGG |
| | | | CGTAAAGAGCACGTAGGTGGTT |
| | | | TTTTAAGTCAGATGTGAAATCCC |
| | | | TAGGCTTAACCTAGGAACTGCA |
| | | | TTTGAAACTGAAATGCTAGAGTA |
| | | | TCGTAGAGGGAGGTAGAATTCT |
| | | | AGGTGTAGCGGTGAAATGCGTA |
| | | | GATATCTGGAGGAATACCCGTG |
| | | | GCGAAAGCGGCCTCCTAAACGA |
| | | | ATACTGACACTGAGGTGCGAAA |
| | | | GCGTGGGGAGCAAACAGGATTA |
| | | | GATACCCTGGTAGTCCATGCCG |
| | | | TAAACGATGTCGACTTGGAGGT |
| | | | TGTTTCCAAGAGAAGTGACTTC |
| | | | CGAAGCTAACGCGTTAAGTCGA |
| | | | CCGCCTGGGGAGTACGGCCGC |
| | | | AAGGCTAAAACTCAAATGAATTG |
| | | | ACGGGGGCCCGCACAAGCGGT |
| | | | GGAGCATGTGGTTTAATTCGAT |
| | | | GCAACGCGAAAAACCTTACCTG |
| | | | GTCTTGACATCCACAGAATTTTT |
| | | | TAGAAATAAAAAAGTGCCTTCG |
| | | | GGAACTGTGAGACAGGTGCTGC |
| | | | ATGGCTGTCGTCAGCTCGTGTT |
| | | | GTGAAATGTTGGGTTAAGTCCC |
| | | | GCAACGAGCGCAACCCTTATCC |
| | | | CCTGTTGCCAGCGGTTCGGCC |
| | | | GGGAACTCAGAGGAGACTGCC |
| | | | GGTTATAAACCGGAGGAAGGTG |
| | | | GGGACGACGTCAAGTCATCATG |
| | | | GCCCTTACGACCAGGGCTACAC |
| | | | ACGTGCTACAATGGTTTATACAA |
| | | | AGAGAAGCAAATCTGTAAAGAC |
| | | | AAGCAAACCTCATAAAGTAAATC |
| | | | GTAGTCCGGACTGGAGTCTGCA |
| | | | ACTCGACTCCACGAAGTCGGAA |
| | | | TCGCTAGTAATCGTGGATCAGA |
| | | | ATGCCACGGTGAATACGTTCCC |
| | | | GGGCCTTGTACACACCGCCCGT |
| | | | CACACCATGGGAGTGGGTTGCA |
| | | | AAAGAAGCAGATTTCCTAACCA |
| | | | CGAAAGTGGAAGGCGTCTACCA |
| | | | CTTTGTGATTCATGACTGGGGT |
| | | | GAAGTCGTAACAAGGTAACCGT |
| | | | AGGGGAACCTGCGGTTGGATCA |
| | | | CCTCCTTA |
| | | | (SEQ ID NO: 4) |
| Buchnera aphidicola str. | Aphids | bacteriocytes | ACTTAAAATTGAAGAGTTTGATC |
| Bp (Baizongia pistaciae) | (Aphidoidea) | | ATGGCTCAGATTGAACGCTGGC |
| | | | GGCAAGCTTAACACATGCAAGT |
| | | | CGAGCGGCATCGAAGAAAAGTT |
| | | | TACTTTTCTGGCGGCGAGCGGC |
| | | | AAACGGGTGAGTAACATCTGGG |
| | | | GATCTACCTAAAAGAGGGGGAC |
| | | | AACCATTGGAAACGATGGCTAA |
| | | | TACCGCATAATGTTTTTAAATAA |
| | | | ACCAAAGTAGGGGACTAAAATT |
| | | | TTTAGCCTTATGCTTTTAGATGA |
| | | | ACCCAGACGAGATTAGCTTGAT |
| | | | GGTAAGGTAATGGCTTACCAAG |
| | | | GCGACGATCTCTAGCTGGTCTG |
| | | | AGAGGATAACCAGCCACACTGG |
| | | | AACTGAGATACGGTCCAGACTC |
| | | | CTACGGGAGGCAGCAGTGGGG |
| | | | AATATTGCACAATGGGCTAAAG |
| | | | CCTGATGCAGCTATGCCGCGTG |
| | | | TATGAAGAAGGCCTTAGGGTTG |
| | | | TAAAGTACTTTCAGCGGGGAGG |
| | | | AAAGAATTATGTCTAATATACAT |
| | | | ATTTTGTGACGTTACCCGAAGA |
| | | | AGAAGCACCGGCTAACTCCGTG |
| | | | CCAGCAGCCGCGGTAATACGG |
| | | | AGGGTGCGAGCGTTAATCAGAA |
| | | | TTACTGGGCGTAAAGAGCACGT |
| | | | AGGCGGTTTATTAAGTCAGATG |
| | | | TGAAATCCCTAGGCTTAACTTAG |
| | | | GAACTGCATTTGAAACTAATAGA |
| | | | CTAGAGTCTCATAGAGGGAGGT |
| | | | AGAATTCTAGGTGTAGCGGTGA |
| | | | AATGCGTAGATATCTAGAGGAA |
| | | | TACCCGTGGCGAAAGCGACCTC |
| | | | CTAAATGAAAACTGACGCTGAG |
| | | | GTGCGAAAGCGTGGGGAGCAA |
| | | | ACAGGATTAGATACCCTGGTAG |
| | | | TCCATGCTGTAAACGATGTCGA |
| | | | CTTGGAGGTTGTTTCCTAGAGA |
| | | | AGTGGCTTCCGAAGCTAACGCA |
| | | | TTAAGTCGACCGCCTGGGGAGT |
| | | | ACGGTCGCAAGGCTAAAACTCA |
| | | | AATGAATTGACGGGGGCCCGCA |
| | | | CAAGCGGTGGAGCATGTGGTTT |
| | | | AATTCGATGCAACGCGAAGAAC |
| | | | CTTACCTGGTCTTGACATCCATA |
| | | | GAATTTTTTAGAGATAAAAGAGT |
| | | | GCCTTAGGGAACTATGAGACAG |
| | | | GTGCTGCATGGCTGTCGTCAGC |
| | | | TCGTGTTGTGAAATGTTGGGTT |
| | | | AAGTCCCGCAACGAGCGCAACC |
| | | | CCTATCCTTTGTTGCCATCAGGT |
| | | | TATGCTGGGAACTCAGAGGAGA |
| | | | CTGCCGGTTATAAACCGGAGGA |
| | | | AGGTGGGGATGACGTCAAGTCA |
| | | | TCATGGCCCTTACGACCAGGGC |
| | | | TACACACGTGCTACAATGGCAT |
| | | | ATACAAAGAGATGCAACTCTGC |
| | | | GAAGATAAGCAAACCTCATAAA |
| | | | GTATGTCGTAGTCCGGACTGGA |
| | | | GTCTGCAACTCGACTCCACGAA |
| | | | GTAGGAATCGCTAGTAATCGTG |
| | | | GATCAGAATGCCACGGTGAATA |
| | | | CGTTCCCGGGCCTTGTACACAC |
| | | | CGCCCGTCACACCATGGGAGT |
| | | | GGGTTGCAAAAGAAGCAGGTAG |
| | | | CTTAACCAGATTATTTTATTGGA |
| | | | GGGCGCTTACCACTTTGTGATT |
| | | | CATGACTGGGGTGAAGTCGTAA |
| | | | CAAGGTAACCGTAGGGGAACCT |
| | | | GCGGTTGGATCACCTCCTTA |
| | | | (SEQ ID NO: 5) |
| Buchnera aphidicola BCc | Aphids | bacteriocytes | ATGAGATCATTAATATATAAAAA |
| | (Aphidoidea) | | TCATGTTCCAATTAAAAAATTAG |
| | | | GACAAAATTTTTTACAGAATAAA |
| | | | GAAATTATTAATCAGATAATTAA |
| | | | TTTAATAAATATTAATAAAAATGA |
| | | | TAATATTATTGAAATAGGATCAG |
| | | | GATTAGGAGCGTTAACTTTTCCT |
| | | | ATTTGTAGAATCATTAAAAAAAT |
| | | | GATAGTATTAGAAATTGATGAAG |
| | | | ATCTTGTGTTTTTTTTAACTCAAA |
| | | | GTTTATTTATTAAAAAATTACAAA |
| | | | TTATAATTGCTGATATTATAAAAT |
| | | | TTGATTTTTGTTGTTTTTTTTCTT |
| | | | TACAGAAATATAAAAAATATAGG |
| | | | TTTATTGGTAATTTACCATATAAT |
| | | | ATTGCTACTATATTTTTTTTAAAA |
| | | | ACAATTAAATTTCTTTATAATATA |
| | | | ATTGATATGCATTTTATGTTTCA |
| | | | AAAAGAAGTAGCAAAGAGATTA |
| | | | TTAGCTACTCCTGGTACTAAAGA |
| | | | ATATGGTAGATTAAGTATTATTG |
| | | | CACAATATTTTTATAAGATAGAA |
| | | | ACTGTTATTAATGTTAATAAATTT |
| | | | AATTTTTTTCCTACTCCTAAAGT |
| | | | AGATTCTACTTTTTTACGATTTA |
| | | | CTCCTAAATATTTTAATAGTAAA |
| | | | TATAAAATAGATAAACATTTTTCT |
| | | | GTTTTAGAATTAATTACTAGATT |
| | | | TTCTTTTCAACATAGAAGAAAAT |
| | | | TTTTAAATAATAATTTAATATCTT |
| | | | TATTTTCTACAAAAGAATTAATTT |
| | | | CTTTAGATATTGATCCATATTCA |
| | | | AGAGCAGAAAATGTTTCTTTAAT |
| | | | TCAATATTGTAAATTAATGAAAT |
| | | | ATTATTTGAAAAGAAAAATTTTAT |
| | | | GTTTAGATTAA |
| | | | (SEQ ID NO: 6) |
| Buchnera aphidicola | Aphids | bacteriocytes | TTATCTTATTTCACATATACGTA |
| (Cinara tujafilina) | (Aphidoidea) | | ATATTGCGCTGCGTGCACGAGG |
| | | | ATTTTTTTGAATTTCAGATATATT |
| | | | TGGTTTAATACGTTTAATAAAAC |
| | | | GTATTTTTTTTTTTATTTTTCTTA |
| | | | TTTGCAATTCAGTAATAGGAAGT |
| | | | TTTTTAGGTATATTTGGATAATT |
| | | | ACTGTAATTCTTAATAAAGTTTTT |
| | | | TACAATCCTATCTTCAATAGAAT |
| | | | GAAAACTAATAATAGCAATTTTT |
| | | | GATCCGGAATGTAATATGTTAAT |
| | | | AATAATTTTTAATATTTTATGTAA |
| | | | TTCATTTATTTCTTGGTTAATATA |
| | | | TATTCGAAAAGCTTGAAATGTTC |
| | | | TCGTAGCTGGATGTTTAAATTTG |
| | | | TCATATTTTGGGATTGATTTTTTT |
| | | | ATGATTTGAACTAACTCTAACGT |
| | | | GCTTGTTATGGTTTTTTTTTTTAT |
| | | | TTGTAATATGATGGCTCGGGAT |
| | | | ATTTTTTTTGCGTATTTTTCTTCG |
| | | | CCAAAATTTTTTATTACCTGTTC |
| | | | TATTGTTTTTTGGTTTGTTTTTTT |
| | | | TAACCATTGACTAACTGATATTC |
| | | | CAGATTTAGGGTTCATACGCAT |
| | | | ATCTAAAGGTCCATCATTCATAA |
| | | | ATGAAAATCCTCGGATACTAGA |
| | | | ATTTAACTGTATTGAAGAAATAC |
| | | | CTAAATCTAATAATATTCCATCT |
| | | | ATTTTATCTCTATTTTTTTCTTTT |
| | | | TTTAATATTTTTTCAATATTAGAA |
| | | | AATTTACCTAAAAATATTTTAAAT |
| | | | CGCGAATCTTTTATTTTTTTTCC |
| | | | GATTTTTATAGATTGTGGGTCTT |
| | | | GATCAATACTATATAACTTTCCA |
| | | | TTAACCCCTAATTCTTGAAGAAT |
| | | | TGCTTTTGAATGACCACCACCT |
| | | | CCAAATGTACAATCAACATATGT |
| | | | ACCGTCTTTTTTTATTTTTAAGTA |
| | | | TTGTATGATTTCTTTTGTTAAAA |
| | | | CAGGTTTATGAATCAT |
| | | | (SEQ ID NO: 7) |
| Buchnera aphidicola str. | Aphids | bacteriocytes | ATGAAAAGTATAAAAACTTTTAA |
| G002 (Myzus persicae) | (Aphidoidea) | | AAAACACTTTCCTGTGAAAAAAT |
| | | | ATGGACAAAATTTTCTTATTAAT |
| | | | AAAGAGATCATAAAAAATATTGT |
| | | | TAAAAAAATTAATCCAAATATAG |
| | | | AACAAACATTAGTAGAAATCGG |
| | | | ACCAGGATTAGCTGCATTAACT |
| | | | GAGCCCATATCTCAGTTATTAAA |
| | | | AGAGTTAATAGTTATTGAAATAG |
| | | | ACTGTAATCTATTATATTTTTTAA |
| | | | AAAAACAACCATTTTATTCAAAA |
| | | | TTAATAGTTTTTTGTCAAGATGC |
| | | | TTTAAACTTTAATTATACAAATTT |
| | | | ATTTTATAAAAAAAATAAATTAAT |
| | | | TCGTATTTTTGGTAATTTACCAT |
| | | | ATAATATCTCTACATCTTTAATTA |
| | | | TTTTTTTATTTCAACACATTAGA |
| | | | GTAATTCAAGATATGAATTTTAT |
| | | | GCTTCAAAAAGAAGTTGCTGCA |
| | | | AGATTAATTGCATTACCTGGAAA |
| | | | TAAATATTACGGTCGTTTGAGCA |
| | | | TTATATCTCAATATTATTGTGATA |
| | | | TCAAAATTTTATTAAATGTTGCT |
| | | | CCTGAAGATTTTTGGCCTATTCC |
| | | | GAGAGTTCATTCTATATTTGTAA |
| | | | ATTTAACACCTCATCATAATTCT |
| | | | CCTTATTTTGTTTATGATATTAAT |
| | | | ATTTTAAGCCTTATTACAAATAA |
| | | | GGCTTTCCAAAATAGAAGAAAA |
| | | | ATATTACGTCATAGTTTAAAAAA |
| | | | TTTATTTTCTGAAACAACTTTATT |
| | | | AAATTTAGATATTAATCCCAGAT |
| | | | TAAGAGCTGAAAATATTTCTGTT |
| | | | TTTCAGTATTGTCAATTAGCTAA |
| | | | TTATTTGTATAAAAAAAATTATAC |
| | | | TAAAAAAAATTAA |
| | | | (SEQ ID NO: 8) |
| Buchnera aphidicola str. | Aphids | bacteriocytes | ATTATAAAAAATTTTAAAAAACAT |
| Ak (Acyrthosiphon | (Aphidoidea) | | TTTCCTTTAAAAAGGTATGGACA |
| kondoi) | | | AAATTTTCTTGTCAATACAAAAA |
| | | | CTATTCAAAAGATAATTAATATA |
| | | | ATTAATCCAAACACCAAACAAAC |
| | | | ATTAGTGGAAATTGGACCTGGA |
| | | | TTAGCTGCATTAACAAAACCAAT |
| | | | TTGTCAATTATTAGAAGAATTAA |
| | | | TTGTTATTGAAATAGATCCTAAT |
| | | | TTATTGTTTTTATTAAAAAAACGT |
| | | | TCATTTTATTCAAAATTAACAGTT |
| | | | TTTTATCAAGACGCTTTAAATTT |
| | | | CAATTATACAGATTTGTTTTATA |
| | | | AGAAAAATCAATTAATTCGTGTT |
| | | | TTTGGAAACTTGCCATATAATAT |
| | | | TTCTACATCTTTAATTATTTCTTT |
| | | | ATTCAATCATATTAAAGTTATTC |
| | | | AAGATATGAATTTTATGTTACAG |
| | | | AAAGAGGTTGCTGAAAGATTAA |
| | | | TTTCTATTCCTGGAAATAAATCT |
| | | | TATGGCCGTTTAAGCATTATTTC |
| | | | TCAGTATTATTGTAAAATTAAAA |
| | | | TATTATTAAATGTTGTACCTGAA |
| | | | GATTTTCGACCTATACCGAAAGT |
| | | | GCATTCTGTTTTTATCAATTTAA |
| | | | CTCCTCATACCAATTCTCCATAT |
| | | | TTTGTTTATGATACAAATATCCT |
| | | | CAGTTCTATCACAAGAAATGCTT |
| | | | TTCAAAATAGAAGGAAAATTTTG |
| | | | CGTCATAGTTTAAAAAATTTATT |
| | | | TTCTGAAAAAGAACTAATTCAAT |
| | | | TAGAAATTAATCCAAATTTACGA |
| | | | GCTGAAAATATTTCTATCTTTCA |
| | | | GTATTGTCAATTAGCTGATTATT |
| | | | TATATAAAAAATTAAATAATCTTG |
| | | | TAAAAATCAATTAA |
| | | | (SEQ ID NO: 9) |
| Buchnera aphidicola str. | Aphids | bacteriocytes | ATGATACTAAATAAATATAAAAA |
| Ua (Uroleucon | (Aphidoidea) | | ATTTATTCCTTTAAAAAGATACG |
| ambrosiae) | | | GACAAAATTTTCTTGTAAATAGA |
| | | | GAAATAATCAAAAATATTATCAA |
| | | | AATAATTAATCCTAAAAAAACGC |
| | | | AAACATTATTAGAAATTGGACCG |
| | | | GGTTTAGGTGCGTTAACAAAAC |
| | | | CTATTTGTGAATTTTTAAATGAA |
| | | | CTTATCGTCATTGAAATAGATCC |
| | | | TAATATATTATCTTTTTTAAAGAA |
| | | | ATGTATATTTTTTGATAAATTAAA |
| | | | AATATATTGTCATAATGCTTTAG |
| | | | ATTTTAATTATAAAAATATATTCT |
| | | | ATAAAAAAAGTCAATTAATTCGT |
| | | | ATTTTTGGAAATTTACCATATAA |
| | | | TATTTCTACATCTTTAATAATATA |
| | | | TTTATTTCGGAATATTGATATTAT |
| | | | TCAAGATATGAATTTTATGTTAC |
| | | | AACAAGAAGTGGCTAAAAGATT |
| | | | AGTTGCTATTCCTGGTGAAAAA |
| | | | CTTTATGGTCGTTTAAGTATTAT |
| | | | ATCTCAATATTATTGTAATATTAA |
| | | | AATATTATTACATATTCGACCTG |
| | | | AAAATTTTCAACCTATTCCTAAA |
| | | | GTTAATTCAATGTTTGTAAATTT |
| | | | AACTCCGCATATTCATTCTCCTT |
| | | | ATTTTGTTTATGATATTAATTTAT |
| | | | TAACTAGTATTACAAAACATGCT |
| | | | TTTCAACATAGAAGAAAAATATT |
| | | | GCGTCATAGTTTAAGAAATTTTT |
| | | | TTTCTGAGCAAGATTTAATTCAT |
| | | | TTAGAAATTAATCCAAATTTAAG |
| | | | AGCTGAAAATGTTTCTATTATTC |
| | | | AATATTGTCAATTGGCTAATAAT |
| | | | TTATATAAAAAACATAAACAGTT |
| | | | TATTAATAATTAA |
| | | | (SEQ ID NO: 10) |
| Buchnera aphidicola | Aphids | bacteriocytes | ATGAAAAAGCATATTCCTATAAA |
| (Aphis glycines) | (Aphidoidea) | | AAAATTTAGTCAAAATTTTCTTG |
| | | | TAGATTTGAGTGTGATTAAAAAA |
| | | | ATAATTAAATTTATTAATCCGCA |
| | | | GTTAAATGAAATATTGGTTGAAA |
| | | | TTGGACCGGGATTAGCTGCTAT |
| | | | CACTCGACCTATTTGTGATTTGA |
| | | | TAGATCATTTAATTGTGATTGAA |
| | | | ATTGATAAAATTTTATTAGATAG |
| | | | ATTAAAACAGTTCTCATTTTATT |
| | | | CAAAATTAACAGTATATCATCAA |
| | | | GATGCTTTAGCATTTGATTACAT |
| | | | AAAGTTATTTAATAAAAAAAATA |
| | | | AATTAGTTCGAATTTTTGGTAAT |
| | | | TTACCATATCATGTTTCTACGTC |
| | | | TTTAATATTGCATTTATTTAAAAG |
| | | | AATTAATATTATTAAAGATATGA |
| | | | ATTTTATGCTACAAAAAGAAGTT |
| | | | GCTGAACGTTTAATTGCAACTC |
| | | | CAGGTAGTAAATTATATGGTCGT |
| | | | TTAAGTATTATTTCTCAATATTAT |
| | | | TGTAATATAAAAGTTTTATTGCA |
| | | | TGTGTCTTCAAAATGTTTTAAAC |
| | | | CAGTTCCTAAAGTAGAATCAATT |
| | | | TTTCTTAATTTGACACCTTATAC |
| | | | TGATTATTTCCCTTATTTTACTTA |
| | | | TAATGTAAACGTTCTTAGTTATA |
| | | | TTACAAATTTAGCTTTTCAAAAA |
| | | | AGAAGAAAAATATTACGTCATAG |
| | | | TTTAGGTAAAATATTTTCTGAAA |
| | | | AAGTTTTTATAAAATTAAATATTA |
| | | | ATCCCAAATTAAGACCTGAGAAT |
| | | | ATTTCTATATTACAATATTGTCA |
| | | | GTTATCTAATTATATGATAGAAA |
| | | | ATAATATTCATCAGGAACATGTT |
| | | | TGTATTTAA |
| | | | (SEQ ID NO: 11) |
| Annandia pinicola | (Phylloxeroidea) | bacteriocytes | AGATTGAACGCTGGCGGCATGC |
| | | | CTTACACATGCAAGTCGAACGG |
| | | | TAACAGGTCTTCGGACGCTGAC |
| | | | GAGTGGCGAACGGGTGAGTAAT |
| | | | ACATCGGAACGTGCCCAGTCGT |
| | | | GGGGGATAACTACTCGAAAGAG |
| | | | TAGCTAATACCGCATACGATCT |
| | | | GAGGATGAAAGCGGGGGACCT |
| | | | TCGGGCCTCGCGCGATTGGAG |
| | | | CGGCCGATGGCAGATTAGGTAG |
| | | | TTGGTGGGATAAAAGCTTACCA |
| | | | AGCCGACGATCTGTAGCTGGTC |
| | | | TGAGAGGACGACCAGCCACACT |
| | | | GGAACTGAGATACGGTCCAGAC |
| | | | TCTTACGGGAGGCAGCAGTGG |
| | | | GGAATATTGCACAATGGGCGCA |
| | | | AGCCTGATGCAGCTATGTCGCG |
| | | | TGTATGAAGAAGACCTTAGGGT |
| | | | TGTAAAGTACTTTCGATAGCATA |
| | | | AGAAGATAATGAGACTAATAATT |
| | | | TTATTGTCTGACGTTAGCTATAG |
| | | | AAGAAGCACCGGCTAACTCCGT |
| | | | GCCAGCAGCCGCGGTAATACG |
| | | | GGGGGTGCTAGCGTTAATCGGA |
| | | | ATTACTGGGCGTAAAGAGCATG |
| | | | TAGGTGGTTTATTAAGTCAGATG |
| | | | TGAAATCCCTGGACTTAATCTAG |
| | | | GAACTGCATTTGAAACTAATAG |
| | | | GCTAGAGTTTCGTAGAGGGAGG |
| | | | TAGAATTCTAGGTGTAGCGGTG |
| | | | AAATGCATAGATATCTAGAGGA |
| | | | ATATCAGTGGCGAAGGCGACCT |
| | | | TCTGGACGATAACTGACGCTAA |
| | | | AATGCGAAAGCATGGGTAGCAA |
| | | | ACAGGATTAGATACCCTGGTAG |
| | | | TCCATGCTGTAAACGATGTCGA |
| | | | CTAAGAGGTTGGAGGTATAACT |
| | | | TTTAATCTCTGTAGCTAACGCGT |
| | | | TAAGTCGACCGCCTGGGGAGTA |
| | | | CGGTCGCAAGGCTAAAACTCAA |
| | | | ATGAATTGACGGGGGCCTGCAC |
| | | | AAGCGGTGGAGCATGTGGTTTA |
| | | | ATTCGATGCAACGCGTAAAACC |
| | | | TTACCTGGTCTTGACATCCACA |
| | | | GAATTTTACAGAAATGTAGAAGT |
| | | | GCAATTTGAACTGTGAGACAGG |
| | | | TGCTGCATGGCTGTCGTCAGCT |
| | | | CGTGTTGTGAAATGTTGGGTTA |
| | | | AGTCCCGCAACGAGCGCAACC |
| | | | CTTGTCCTTTGTTACCATAAGAT |
| | | | TTAAGGAACTCAAAGGAGACTG |
| | | | CCGGTGATAAACTGGAGGAAGG |
| | | | CGGGGACGACGTCAAGTCATCA |
| | | | TGGCCCTTATGACCAGGGCTAC |
| | | | ACACGTGCTACAATGGCATATA |
| | | | CAAAGAGATGCAATATTGCGAA |
| | | | ATAAAGCCAATCTTATAAAATAT |
| | | | GTCCTAGTTCGGACTGGAGTCT |
| | | | GCAACTCGACTCCACGAAGTCG |
| | | | GAATCGCTAGTAATCGTGGATC |
| | | | AGCATGCCACGGTGAATATGTT |
| | | | TCCAGGCCTTGTACACACCGCC |
| | | | CGTCACACCATGGAAGTGGATT |
| | | | GCAAAAGAAGTAAGAAAATTAA |
| | | | CCTTCTTAACAAGGAAATAACTT |
| | | | ACCACTTTGTGACTCATAACTG |
| | | | GGGTGA |
| | | | (SEQ ID NO: 12) |
| Moranella endobia | (Coccoidea) | bacteriocytes | TCTTTTTGGTAAGGAGGTGATC |
| | | | CAACCGCAGGTTCCCCTACGGT |
| | | | TACCTTGTTACGACTTCACCCCA |
| | | | GTCATGAATCACAAAGTGGTAA |
| | | | GCGCCCTCCTAAAAGGTTAGGC |
| | | | TACCTACTTCTTTTGCAACCCAC |
| | | | TTCCATGGTGTGACGGGCGGTG |
| | | | TGTACAAGGCCCGGGAACGTAT |
| | | | TCACCGTGGCATTCTGATCCAC |
| | | | GATTACTAGCGATTCCTACTTCA |
| | | | TGGAGTCGAGTTGCAGACTCCA |
| | | | ATCCGGACTACGACGCACTTTA |
| | | | TGAGGTCCGCTAACTCTCGCGA |
| | | | GCTTGCTTCTCTTTGTATGCGC |
| | | | CATTGTAGCACGTGTGTAGCCC |
| | | | TACTCGTAAGGGCCATGATGAC |
| | | | TTGACGTCATCCCCACCTTCCT |
| | | | CCGGTTTATCACCGGCAGTCTC |
| | | | CTTTGAGTTCCCGACCGAATCG |
| | | | CTGGCAAAAAAGGATAAGGGTT |
| | | | GCGCTCGTTGCGGGACTTAACC |
| | | | CAACATTTCACAACACGAGCTG |
| | | | ACGACAGCCATGCAGCACCTGT |
| | | | CTCAGAGTTCCCGAAGGTACCA |
| | | | AAACATCTCTGCTAAGTTCTCTG |
| | | | GATGTCAAGAGTAGGTAAGGTT |
| | | | CTTCGCGTTGCATCGAATTAAA |
| | | | CCACATGCTCCACCGCTTGTGC |
| | | | GGGCCCCCGTCAATTCATTTGA |
| | | | GTTTTAACCTTGCGGCCGTACT |
| | | | CCCCAGGCGGTCGATTTAACGC |
| | | | GTTAACTACGAAAGCCACAGTT |
| | | | CAAGACCACAGCTTTCAAATCG |
| | | | ACATAGTTTACGGCGTGGACTA |
| | | | CCAGGGTATCTAATCCTGTTTG |
| | | | CTCCCCACGCTTTCGTACCTGA |
| | | | GCGTCAGTATTCGTCCAGGGGG |
| | | | CCGCCTTCGCCACTGGTATTCC |
| | | | TCCAGATATCTACACATTTCACC |
| | | | GCTACACCTGGAATTCTACCCC |
| | | | CCTCTACGAGACTCTAGCCTAT |
| | | | CAGTTTCAAATGCAGTTCCTAG |
| | | | GTTAAGCCCAGGGATTTCACAT |
| | | | CTGACTTAATAAACCGCCTACG |
| | | | TACTCTTTACGCCCAGTAATTCC |
| | | | GATTAACGCTTGCACCCTCCGT |
| | | | ATTACCGCGGCTGCTGGCACG |
| | | | GAGTTAGCCGGTGCTTCTTCTG |
| | | | TAGGTAACGTCAATCAATAACC |
| | | | GTATTAAGGATATTGCCTTCCTC |
| | | | CCTACTGAAAGTGCTTTACAAC |
| | | | CCGAAGGCCTTCTTCACACACG |
| | | | CGGCATGGCTGCATCAGGGTTT |
| | | | CCCCCATTGTGCAATATTCCCC |
| | | | ACTGCTGCCTCCCGTAGGAGTC |
| | | | TGGACCGTGTCTCAGTTCCAGT |
| | | | GTGGCTGGTCATCCTCTCAGAC |
| | | | CAGCTAGGGATCGTCGCCTAGG |
| | | | TAAGCTATTACCTCACCTACTAG |
| | | | CTAATCCCATCTGGGTTCATCT |
| | | | GAAGGTGTGAGGCCAAAAGGTC |
| | | | CCCCACTTTGGTCTTACGACATT |
| | | | ATGCGGTATTAGCTACCGTTTC |
| | | | CAGCAGTTATCCCCCTCCATCA |
| | | | GGCAGATCCCCAGACTTTACTC |
| | | | ACCCGTTCGCTGCTCGCCGGCA |
| | | | AAAAAGTAAACTTTTTTCCGTTG |
| | | | CCGCTCAACTTGCATGTGTTAG |
| | | | GCCTGCCGCCAGCGTTCAATCT |
| | | | GAGCCATGATCAAACTCTTCAAT |
| | | | TAAA |
| | | | (SEQ ID NO: 13) |
| Ishikawaella capsulata | (Heteroptera) | bacteriocytes | AAATTGAAGAGTTTGATCATGG |
| Mpkobe | | | CTCAGATTGAACGCTAGCGGCA |
| | | | AGCTTAACACATGCAAGTCGAA |
| | | | CGGTAACAGAAAAAAGCTTGCT |
| | | | TTTTTGCTGACGAGTGGCGGAC |
| | | | GGGTGAGTAATGTCTGGGGATC |
| | | | TACCTAATGGCGGGGGATAACT |
| | | | ACTGGAAACGGTAGCTAATACC |
| | | | GCATAATGTTGTAAAACCAAAGT |
| | | | GGGGGACCTTATGGCCTCACAC |
| | | | CATTAGATGAACCTAGATGGGA |
| | | | TTAGCTTGTAGGTGGGGTAAAG |
| | | | GCTCACCTAGGCAACGATCCCT |
| | | | AGCTGGTCTGAGAGGATGACCA |
| | | | GCCACACTGGAACTGAGATACG |
| | | | GTCCAGACTCCTACGGGAGGCA |
| | | | GCAGTGGGGAATCTTGCACAAT |
| | | | GGGCGCAAGCCTGATGCAGCT |
| | | | ATGTCGCGTGTATGAAGAAGGC |
| | | | CTTAGGGTTGTAAAGTACTTTCA |
| | | | TCGGGGAAGAAGGATATGAGCC |
| | | | TAATATTCTCATATATTGACGTT |
| | | | ACCTGCAGAAGAAGCACCGGCT |
| | | | AACTCCGTGCCAGCAGCCGCG |
| | | | GTAACACGGAGGGTGCGAGCG |
| | | | TTAATCGGAATTACTGGGCGTA |
| | | | AAGAGCACGTAGGTGGTTTATT |
| | | | AAGTCATATGTGAAATCCCTGG |
| | | | GCTTAACCTAGGAACTGCATGT |
| | | | GAAACTGATAAACTAGAGTTTC |
| | | | GTAGAGGGAGGTGGAATTCCAG |
| | | | GTGTAGCGGTGAAATGCGTAGA |
| | | | TATCTGGAGGAATATCAGAGGC |
| | | | GAAGGCGACCTTCTGGACGAAA |
| | | | ACTGACACTCAGGTGCGAAAGC |
| | | | GTGGGGAGCAAACAGGATTAGA |
| | | | TACCCTGGTAGTCCACGCTGTA |
| | | | AACAATGTCGACTAAAAAACTGT |
| | | | GAGCTTGACTTGTGGTTTTTGTA |
| | | | GCTAACGCATTAAGTCGACCGC |
| | | | CTGGGGAGTACGGCCGCAAGG |
| | | | TTAAAACTCAAATGAATTGACGG |
| | | | GGGTCCGCACAAGCGGTGGAG |
| | | | CATGTGGTTTAATTCGATGCAAC |
| | | | GCGAAAAACCTTACCTGGTCTT |
| | | | GACATCCAGCGAATTATATAGA |
| | | | AATATATAAGTGCCTTTCGGGG |
| | | | AACTCTGAGACGCTGCATGGCT |
| | | | GTCGTCAGCTCGTGTTGTGAAA |
| | | | TGTTGGGTTAAGTCCCGCAACG |
| | | | AGCGCCCTTATCCTCTGTTGCC |
| | | | AGCGGCATGGCCGGGAACTCA |
| | | | GAGGAGACTGCCAGTATTAAAC |
| | | | TGGAGGAAGGTGGGGATGACG |
| | | | TCAAGTCATCATGGCCCTTATG |
| | | | ACCAGGGCTACACACGTGCTAC |
| | | | AATGGTGTATACAAAGAGAAGC |
| | | | AATCTCGCAAGAGTAAGCAAAA |
| | | | CTCAAAAAGTACATCGTAGTTC |
| | | | GGATTAGAGTCTGCAACTCGAC |
| | | | TCTATGAAGTAGGAATCGCTAG |
| | | | TAATCGTGGATCAGAATGCCAC |
| | | | GGTGAATACGTTCTCTGGCCTT |
| | | | GTACACACCGCCCGTCACACCA |
| | | | TGGGAGTAAGTTGCAAAAGAAG |
| | | | TAGGTAGCTTAACCTTTATAGGA |
| | | | GGGCGCTTACCACTTTGTGATT |
| | | | TATGACTGGGGTGAAGTCGTAA |
| | | | CAAGGTAACTGTAGGGGAACCT |
| | | | GTGGTTGGATTACCTCCTTA |
| | | | (SEQ ID NO: 14) |
| Baumannia | sharpshooter | bacteriocytes | TTCAATTGAAGAGTTTGATCATG |
| cicadellinicola | leafhoppers | | GCTCAGATTGAACGCTGGCGGT |
| | (Cicadellinae) | | AAGCTTAACACATGCAAGTCGA |
| | | | GCGGCATCGGAAAGTAAATTAA |
| | | | TTACTTTGCCGGCAAGCGGCGA |
| | | | ACGGGTGAGTAATATCTGGGGA |
| | | | TCTACCTTATGGAGAGGGATAA |
| | | | CTATTGGAAACGATAGCTAACA |
| | | | CCGCATAATGTCGTCAGACCAA |
| | | | AATGGGGGACCTAATTTAGGCC |
| | | | TCATGCCATAAGATGAACCCAG |
| | | | ATGAGATTAGCTAGTAGGTGAG |
| | | | ATAATAGCTCACCTAGGCAACG |
| | | | ATCTCTAGTTGGTCTGAGAGGA |
| | | | TGACCAGCCACACTGGAACTGA |
| | | | GACACGGTCCAGACTCCTACGG |
| | | | GAGGCAGCAGTGGGGAATCTT |
| | | | GCACAATGGGGGAAACCCTGAT |
| | | | GCAGCTATACCGCGTGTGTGAA |
| | | | GAAGGCCTTCGGGTTGTAAAGC |
| | | | ACTTTCAGCGGGGAAGAAAATG |
| | | | AAGTTACTAATAATAATTGTCAA |
| | | | TTGACGTTACCCGCAAAAGAAG |
| | | | CACCGGCTAACTCCGTGCCAGC |
| | | | AGCCGCGGTAAGACGGAGGGT |
| | | | GCAAGCGTTAATCGGAATTACT |
| | | | GGGCGTAAAGCGTATGTAGGC |
| | | | GGTTTATTTAGTCAGGTGTGAAA |
| | | | GCCCTAGGCTTAACCTAGGAAT |
| | | | TGCATTTGAAACTGGTAAGCTA |
| | | | GAGTCTCGTAGAGGGGGGGAG |
| | | | AATTCCAGGTGTAGCGGTGAAA |
| | | | TGCGTAGAGATCTGGAAGAATA |
| | | | CCAGTGGCGAAGGCGCCCCCC |
| | | | TGGACGAAAACTGACGCTCAAG |
| | | | TACGAAAGCGTGGGGAGCAAAC |
| | | | AGGATTAGATACCCTGGTAGTC |
| | | | CACGCTGTAAACGATGTCGATT |
| | | | TGAAGGTTGTAGCCTTGAGCTA |
| | | | TAGCTTTCGAAGCTAACGCATTA |
| | | | AATCGACCGCCTGGGGAGTAC |
| | | | GACCGCAAGGTTAAAACTCAAA |
| | | | TGAATTGACGGGGGCCCGCAC |
| | | | AAGCGGTGGAGCATGTGGTTTA |
| | | | ATTCGATACAACGCGAAAAACC |
| | | | TTACCTACTCTTGACATCCAGAG |
| | | | TATAAAGCAGAAAAGCTTTAGTG |
| | | | CCTTCGGGAACTCTGAGACAGG |
| | | | TGCTGCATGGCTGTCGTCAGCT |
| | | | CGTGTTGTGAAATGTTGGGTTA |
| | | | AGTCCCGCAACGAGCGCAACC |
| | | | CTTATCCTTTGTTGCCAACGATT |
| | | | AAGTCGGGAACTCAAAGGAGAC |
| | | | TGCCGGTGATAAACCGGAGGAA |
| | | | GGTGAGGATAACGTCAAGTCAT |
| | | | CATGGCCCTTACGAGTAGGGCT |
| | | | ACACACGTGCTACAATGGTGCA |
| | | | TACAAAGAGAAGCAATCTCGTA |
| | | | AGAGTTAGCAAACCTCATAAAG |
| | | | TGCATCGTAGTCCGGATTAGAG |
| | | | TCTGCAACTCGACTCTATGAAG |
| | | | TCGGAATCGCTAGTAATCGTGG |
| | | | ATCAGAATGCCACGGTGAATAC |
| | | | GTTCCCGGGCCTTGTACACACC |
| | | | GCCCGTCACACCATGGGAGTGT |
| | | | ATTGCAAAAGAAGTTAGTAGCTT |
| | | | AACTCATAATACGAGAGGGCGC |
| | | | TTACCACTTTGTGATTCATAACT |
| | | | GGGGTGAAGTCGTAACAAGGTA |
| | | | ACCGTAGGGGAACCTGCGGTT |
| | | | GGATCACCTCCTTACACTAAA |
| | | | (SEQ ID NO: 15) |
| Sodalis like | Rhopalus | wider tissue | ATTGAACGCTGGCGGCAGGCCT |
| | sapporensis | tropism | AACACATGCAAGTCGAGCGGCA |
| | | | GCGGGAAGAAGCTTGCTTCTTT |
| | | | GCCGGCGAGCGGCGGACGGGT |
| | | | GAGTAATGTCTGGGGATCTGCC |
| | | | CGATGGAGGGGGATAACTACTG |
| | | | GAAACGGTAGCTAATACCGCAT |
| | | | AACGTCGCAAGACCAAAGTGGG |
| | | | GGACCTTCGGGCCTCACACCAT |
| | | | CGGATGAACCCAGGTGGGATTA |
| | | | GCTAGTAGGTGGGGTAATGGCT |
| | | | CACCTAGGCGACGATCCCTAGC |
| | | | TGGTCTGAGAGGATGACCAGTC |
| | | | ACACTGGAACTGAGACACGGTC |
| | | | CAGACTCCTACGGGAGGCAGC |
| | | | AGTGGGGAATATTGCACAATGG |
| | | | GGGAAACCCTGATGCAGCCATG |
| | | | CCGCGTGTGTGAAGAAGGCCTT |
| | | | CGGGTTGTAAAGCACTTTCAGC |
| | | | GGGGAGGAAGGCGATGGCGTT |
| | | | AATAGCGCTATCGATTGACGTT |
| | | | ACCCGCAGAAGAAGCACCGGC |
| | | | TAACTCCGTGCCAGCAGCCGCG |
| | | | GTAATACGGAGGGTGCGAGCG |
| | | | TTAATCGGAATTACTGGGCGTA |
| | | | AAGCGTACGCAGGCGGTCTGTT |
| | | | AAGTCAGATGTGAAATCCCCGG |
| | | | GCTCAACCTGGGAACTGCATTT |
| | | | GAAACTGGCAGGCTAGAGTCTC |
| | | | GTAGAGGGGGGTAGAATTCCAG |
| | | | GTGTAGCGGTGAAATGCGTAGA |
| | | | GATCTGGAGGAATACCGGTGGC |
| | | | GAAGGCGGCCCCCTGGACGAA |
| | | | GACTGACGCTCAGGTACGAAAG |
| | | | CGTGGGGAGCAAACAGGATTAG |
| | | | ATACCCTGGTAGTCCACGCTGT |
| | | | AAACGATGTCGATTTGAAGGTT |
| | | | GTGGCCTTGAGCCGTGGCTTTC |
| | | | GGAGCTAACGTGTTAAATCGAC |
| | | | CGCCTGGGGAGTACGGCCGCA |
| | | | AGGTTAAAACTCAAATGAATTGA |
| | | | CGGGGGCCCGCACAAGCGGTG |
| | | | GAGCATGTGGTTTAATTCGATG |
| | | | CAACGCGAAGAACCTTACCTAC |
| | | | TCTTGACATCCAGAGAACTTGG |
| | | | CAGAGATGCTTTGGTGCCTTCG |
| | | | GGAACTCTGAGACAGGTGCTGC |
| | | | ATGGCTGTCGTCAGCTCGTGTT |
| | | | GTGAAATGTTGGGTTAAGTCCC |
| | | | GCAACGAGCGCAACCCTTATCC |
| | | | TTTATTGCCAGCGATTCGGTCG |
| | | | GGAACTCAAAGGAGACTGCCG |
| | | | GTGATAAACCGGAGGAAGGTG |
| | | | GGGATGACGTCAAGTCATCATG |
| | | | GCCCTTACGAGTAGGGCTACAC |
| | | | ACGTGCTACAATGGCGCATACA |
| | | | AAGAGAAGCGATCTCGCGAGAG |
| | | | TCAGCGGACCTCATAAAGTGCG |
| | | | TCGTAGTCCGGATTGGAGTCTG |
| | | | CAACTCGACTCCATGAAGTCGG |
| | | | AATCGCTAGTAATCGTGGATCA |
| | | | GAATGCCACGGTGAATACGTTC |
| | | | CCGGGCCTTGTACACACCGCCC |
| | | | GTCACACCATGGGAGTGGGTTG |
| | | | CAAAAGAAGTAGGTAGCTTAAC |
| | | | CTTCGGGAGGGCGCTTACCACT |
| | | | TTGTGATTCATGACTGGGGTG |
| | | | (SEQ ID NO: 16) |
| Hartigia pinicola | The pine bark | bacteriocytes | AGATTTAACGCTGGCGGCAGGC |
| | adelgid | | CTAACACATGCAAGTCGAGCGG |
| | | | TACCAGAAGAAGCTTGCTTCTT |
| | | | GCTGACGAGCGGCGGACGGGT |
| | | | GAGTAATGTATGGGGATCTGCC |
| | | | CGACAGAGGGGGATAACTATTG |
| | | | GAAACGGTAGCTAATACCGCAT |
| | | | AATCTCTGAGGAGCAAAGCAGG |
| | | | GGAACTTCGGTCCTTGCGCTAT |
| | | | CGGATGAACCCATATGGGATTA |
| | | | GCTAGTAGGTGAGGTAATGGCT |
| | | | CCCCTAGGCAACGATCCCTAGC |
| | | | TGGTCTGAGAGGATGATCAGCC |
| | | | ACACTGGGACTGAGACACGGC |
| | | | CCAGACTCCTACGGGAGGCAG |
| | | | CAGTGGGGAATATTGCACAATG |
| | | | GGCGAAAGCCTGATGCAGCCAT |
| | | | GCCGCGTGTATGAAGAAGGCTT |
| | | | TAGGGTTGTAAAGTACTTTCAGT |
| | | | CGAGAGGAAAACATTGATGCTA |
| | | | ATATCATCAATTATTGACGTTTC |
| | | | CGACAGAAGAAGCACCGGCTAA |
| | | | CTCCGTGCCAGCAGCCGCGGT |
| | | | AATACGGAGGGTGCAAGCGTTA |
| | | | ATCGGAATTACTGGGCGTAAAG |
| | | | CGCACGCAGGCGGTTAATTAAG |
| | | | TTAGATGTGAAAGCCCCGGGCT |
| | | | TAACCCAGGAATAGCATATAAAA |
| | | | CTGGTCAACTAGAGTATTGTAG |
| | | | AGGGGGGTAGAATTCCATGTGT |
| | | | AGCGGTGAAATGCGTAGAGATG |
| | | | TGGAGGAATACCAGTGGCGAAG |
| | | | GCGGCCCCCTGGACAAAAACTG |
| | | | ACGCTCAAATGCGAAAGCGTGG |
| | | | GGAGCAAACAGGATTAGATACC |
| | | | CTGGTAGTCCATGCTGTAAACG |
| | | | ATGTCGATTTGGAGGTTGTTCC |
| | | | CTTGAGGAGTAGCTTCCGTAGC |
| | | | TAACGCGTTAAATCGACCGCCT |
| | | | GGGGGAGTACGACTGCAAGGT |
| | | | TAAAACTCAAATGAATTGACGG |
| | | | GGGCCCGCACAAGCGGTGGAG |
| | | | CATGTGGTTTAATTCGATGCAAC |
| | | | GCGAAAAACCTTACCTACTCTT |
| | | | GACATCCAGATAATTTAGCAGA |
| | | | AATGCTTTAGTACCTTCGGGAA |
| | | | ATCTGAGACAGGTGCTGCATGG |
| | | | CTGTCGTCAGCTCGTGTTGTGA |
| | | | AATGTTGGGTTAAGTCCCGCAA |
| | | | CGAGCGCAACCCTTATCCTTTG |
| | | | TTGCCAGCGATTAGGTCGGGAA |
| | | | CTCAAAGGAGACTGCCGGTGAT |
| | | | AAACCGGAGGAAGGTGGGGAT |
| | | | GACGTCAAGTCATCATGGCCCT |
| | | | TACGAGTAGGGCTACACACGTG |
| | | | CTACAATGGCATATACAAAGGG |
| | | | AAGCAACCTCGCGAGAGCAAGC |
| | | | GAAACTCATAAATTATGTCGTAG |
| | | | TTCAGATTGGAGTCTGCAACTC |
| | | | GACTCCATGAAGTCGGAATCGC |
| | | | TAGTAATCGTAGATCAGAATGCT |
| | | | ACGGTGAATACGTTCCCGGGCC |
| | | | TTGTACACACCGCCCGTCACAC |
| | | | CATGGGAGTGGGTTGCAAAAGA |
| | | | AGTAGGTAACTTAACCTTATGGA |
| | | | AAGCGCTTACCACTTTGTGATTC |
| | | | ATAACTGGGGTG |
| | | | (SEQ ID NO: 17 |
| Wigglesworthia | tsetse fly | bacteriocytes | |
| glossinidia | (Diptera: | | |
| | Glossinidae) | | |
| Betaproteobacteria | | | |
| Tremblaya phenacola | Phenacoccus | bacteriomes | AGGTAATCCAGCCACACCTTCC |
| | avenae | | AGTACGGCTACCTTGTTACGAC |
| | (TPPAVE). | | TTCACCCCAGTCACAACCCTTA |
| | | | CCTTCGGAACTGCCCTCCTCAC |
| | | | AACTCAAACCACCAAACACTTTT |
| | | | AAATCAGGTTGAGAGAGGTTAG |
| | | | GCCTGTTACTTCTGGCAAGAAT |
| | | | TATTTCCATGGTGTGACGGGCG |
| | | | GTGTGTACAAGACCCGAGAACA |
| | | | TATTCACCGTGGCATGCTGATC |
| | | | CACGATTACTAGCAATTCCAACT |
| | | | TCATGCACTCGAGTTTCAGAGT |
| | | | ACAATCCGAACTGAGGCCGGCT |
| | | | TTGTGAGATTAGCTCCCTTTTGC |
| | | | AAGTTGGCAACTCTTTGGTCCG |
| | | | GCCATTGTATGATGTGTGAAGC |
| | | | CCCACCCATAAAGGCCATGAGG |
| | | | ACTTGACGTCATCCCCACCTTC |
| | | | CTCCAACTTATCGCTGGCAGTC |
| | | | TCTTTAAGGTAACTGACTAATCC |
| | | | AGTAGCAATTAAAGACAGGGGT |
| | | | TGCGCTCGTTACAGGACTTAAC |
| | | | CCAACATCTCACGACACGAGCT |
| | | | GACGACAGCCATGCAGCACCTG |
| | | | TGCACTAATTCTCTTTCAAGCAC |
| | | | TCCCGCTTCTCAACAGGATCTT |
| | | | AGCCATATCAAAGGTAGGTAAG |
| | | | GTTTTTCGCGTTGCATCGAATTA |
| | | | ATCCACATCATCCACTGCTTGT |
| | | | GCGGGTCCCCGTCAATTCCTTT |
| | | | GAGTTTTAACCTTGCGGCCGTA |
| | | | CTCCCCAGGCGGTCGACTTGTG |
| | | | CGTTAGCTGCACCACTGAAAAG |
| | | | GAAAACTGCCCAATGGTTAGTC |
| | | | AACATCGTTTAGGGCATGGACT |
| | | | ACCAGGGTATCTAATCCTGTTT |
| | | | GCTCCCCATGCTTTAGTGTCTG |
| | | | AGCGTCAGTAACGAACCAGGAG |
| | | | GCTGCCTACGCTTTCGGTATTC |
| | | | CTCCACATCTCTACACATTTCAC |
| | | | TGCTACATGCGGAATTCTACCT |
| | | | CCCCCTCTCGTACTCCAGCCTG |
| | | | CCAGTAACTGCCGCATTCTGAG |
| | | | GTTAAGCCTCAGCCTTTCACAG |
| | | | CAATCTTAACAGGCAGCCTGCA |
| | | | CACCCTTTACGCCCAATAAATCT |
| | | | GATTAACGCTCGCACCCTACGT |
| | | | ATTACCGCGGCTGCTGGCACGT |
| | | | AGTTTGCCGGTGCTTATTCTTTC |
| | | | GGTACAGTCACACCACCAAATT |
| | | | GTTAGTTGGGTGGCTTTCTTTC |
| | | | CGAACAAAAGTGCTTTACAACC |
| | | | CAAAGGCCTTCTTCACACACGC |
| | | | GGCATTGCTGGATCAGGCTTCC |
| | | | GCCCATTGTCCAAGATTCCTCA |
| | | | CTGCTGCCTTCCTCAGAAGTCT |
| | | | GGGCCGTGTCTCAGTCCCAGTG |
| | | | TGGCTGGCCGTCCTCTCAGACC |
| | | | AGCTACCGATCATTGCCTTGGG |
| | | | AAGCCATTACCTTTCCAACAAG |
| | | | CTAATCAGACATCAGCCAATCT |
| | | | CAGAGCGCAAGGCAATTGGTCC |
| | | | CCTGCTTTCATTCTGCTTGGTAG |
| | | | AGAACTTTATGCGGTATTAATTA |
| | | | GGCTTTCACCTAGCTGTCCCCC |
| | | | ACTCTGAGGCATGTTCTGATGC |
| | | | ATTACTCACCCGTTTGCCACTTG |
| | | | CCACCAAGCCTAAGCCCGTGTT |
| | | | GCCGTTCGACTTGCATGTGTAA |
| | | | GGCATGCCGCTAGCGTTCAATC |
| | | | TGAGCCAGGATCAAACTCT |
| | | | (SEQ ID NO: 18) |
| Tremblaya princeps | citrus mealybug | bacteriomes | AGAGTTTGATCCTGGCTCAGAT |
| | Planococcus citri | | TGAACGCTAGCGGCATGCATTA |
| | | | CACATGCAAGTCGTACGGCAGC |
| | | | ACGGGCTTAGGCCTGGTGGCG |
| | | | AGTGGCGAACGGGTGAGTAAC |
| | | | GCCTCGGAACGTGCCTTGTAGT |
| | | | GGGGGATAGCCTGGCGAAAGC |
| | | | CAGATTAATACCGCATGAAGCC |
| | | | GCACAGCATGCGCGGTGAAAGT |
| | | | GGGGGATTCTAGCCTCACGCTA |
| | | | CTGGATCGGCCGGGGTCTGATT |
| | | | AGCTAGTTGGCGGGGTAATGGC |
| | | | CCACCAAGGCTTAGATCAGTAG |
| | | | CTGGTCTGAGAGGACGATCAGC |
| | | | CACACTGGGACTGAGACACGG |
| | | | CCCAGACTCCTACGGGAGGCA |
| | | | GCAGTGGGGAATCTTGGACAAT |
| | | | GGGCGCAAGCCTGATCCAGCA |
| | | | ATGCCGCGTGTGTGAAGAAGGC |
| | | | CTTCGGGTCGTAAAGCACTTTT |
| | | | GTTCGGGATGAAGGGGGGCGT |
| | | | GCAAACACCATGCCCTCTTGAC |
| | | | GATACCGAAAGAATAAGCACCG |
| | | | GCTAACTACGTGCCAGCAGCCG |
| | | | CGGTAATACGTAGGGTGCGAGC |
| | | | GTTAATCGGAATCACTGGGCGT |
| | | | AAAGGGTGCGCGGGTGGTTTG |
| | | | CCAAGACCCCTGTAAAATCCTA |
| | | | CGGCCCAACCGTAGTGCTGCG |
| | | | GAGGTTACTGGTAAGCTTGAGT |
| | | | ATGGCAGAGGGGGGTAGAATTC |
| | | | CAGGTGTAGCGGTGAAATGCGT |
| | | | AGATATCTGGAGGAATACCGAA |
| | | | GGCGAAGGCAACCCCCTGGGC |
| | | | CATCACTGACACTGAGGCACGA |
| | | | AAGCGTGGGGAGCAAACAGGA |
| | | | TTAGATACCCTGGTAGTCCACG |
| | | | CCCTAAACCATGTCGACTAGTT |
| | | | GTCGGGGGGAGCCCTTTTTCCT |
| | | | CGGTGACGAAGCTAACGCATGA |
| | | | AGTCGACCGCCTGGGGAGTAC |
| | | | GACCGCAAGGTTAAAACTCAAA |
| | | | GGAATTGACGGGGACCCGCAC |
| | | | AAGCGGTGGATGATGTGGATTA |
| | | | ATTCGATGCAACGCGAAAAACC |
| | | | TTACCTACCCTTGACATGGCGG |
| | | | AGATTCTGCCGAGAGGCGGAA |
| | | | GTGCTCGAAAGAGAATCCGTGC |
| | | | ACAGGTGCTGCATGGCTGTCGT |
| | | | CAGCTCGTGTCGTGAGATGTTG |
| | | | GGTTAAGTCCCATAACGAGCGC |
| | | | AACCCCCGTCTTTAGTTGCTAC |
| | | | CACTGGGGCACTCTATAGAGAC |
| | | | TGCCGGTGATAAACCGGAGGAA |
| | | | GGTGGGGACGACGTCAAGTCAT |
| | | | CATGGCCTTTATGGGTAGGGCT |
| | | | TCACACGTCATACAATGGCTGG |
| | | | AGCAAAGGGTCGCCAACTCGAG |
| | | | AGAGGGAGCTAATCCCACAAAC |
| | | | CCAGCCCCAGTTCGGATTGCAC |
| | | | TCTGCAACTCGAGTGCATGAAG |
| | | | TCGGAATCGCTAGTAATCGTGG |
| | | | ATCAGCATGCCACGGTGAATAC |
| | | | GTTCTCGGGTCTTGTACACACC |
| | | | GCCCGTCACACCATGGGAGTAA |
| | | | GCCGCATCAGAAGCAGCCTCCC |
| | | | TAACCCTATGCTGGGAAGGAGG |
| | | | CTGCGAAGGTGGGGTCTATGAC |
| | | | TGGGGTGAAGTCGTAACAAGGT |
| | | | AGCCGTACCGGAAGGTGCGGC |
| | | | TGGATTACCT |
| | | | (SEQ ID NO: 19) |
| Vidania | | bacteriomes | |
| Nasuia deltocephalinicola | pestiferous insect | bacteriomes | AGTTTAATCCTGGCTCAGATTTA |
| | host, Macrosteles | | ACGCTTGCGACATGCCTAACAC |
| | quadripunctulatus | | ATGCAAGTTGAACGTTGAAAATA |
| | (Hemiptera: | | TTTCAAAGTAGCGTATAGGTGA |
| | Cicadellidae) | | GTATAACATTTAAACATACCTTA |
| | | | AAGTTCGGAATACCCCGATGAA |
| | | | AATCGGTATAATACCGTATAAAA |
| | | | GTATTTAAGAATTAAAGCGGGG |
| | | | AAAACCTCGTGCTATAAGATTGT |
| | | | TAAATGCCTGATTAGTTTGTTGG |
| | | | TTTTTAAGGTAAAAGCTTACCAA |
| | | | GACTTTGATCAGTAGCTATTCTG |
| | | | TGAGGATGTATAGCCACATTGG |
| | | | GATTGAAATAATGCCCAAACCT |
| | | | CTACGGAGGGCAGCAGTGGGG |
| | | | AATATTGGACAATGAGCGAAAG |
| | | | CTTGATCCAGCAATGTCGCGTG |
| | | | TGCGATTAAGGGAAACTGTAAA |
| | | | GCACTTTTTTTTAAGAATAAGAA |
| | | | ATTTTAATTAATAATTAAAATTTT |
| | | | TGAATGTATTAAAAGAATAAGTA |
| | | | CCGACTAATCACGTGCCAGCAG |
| | | | TCGCGGTAATACGTGGGGTGC |
| | | | GAGCGTTAATCGGATTTATTGG |
| | | | GCGTAAAGTGTATTCAGGCTGC |
| | | | TTAAAAAGATTTATATTAAATATT |
| | | | TAAATTAAATTTAAAAAATGTATA |
| | | | AATTACTATTAAGCTAGAGTTTA |
| | | | GTATAAGAAAAAAGAATTTTATG |
| | | | TGTAGCAGTGAAATGCGTTGAT |
| | | | ATATAAAGGAACGCCGAAAGCG |
| | | | AAAGCATTTTTCTGTAATAGAAC |
| | | | TGACGCTTATATACGAAAGCGT |
| | | | GGGTAGCAAACAGGATTAGATA |
| | | | CCCTGGTAGTCCACGCCCTAAA |
| | | | CTATGTCAATTAACTATTAGAAT |
| | | | TTTTTTTAGTGGTGTAGCTAACG |
| | | | CGTTAAATTGACCGCCTGGGTA |
| | | | TTACGATCGCAAGATTAAAACTC |
| | | | AAAGGAATTGACGGGGACCAGC |
| | | | ACAAGCGGTGGATGATGTGGAT |
| | | | TAATTCGATGATACGCGAAAAA |
| | | | CCTTACCTGCCCTTGACATGGT |
| | | | TAGAATTTTATTGAAAAATAAAA |
| | | | GTGCTTGGAAAAGAGCTAACAC |
| | | | ACAGGTGCTGCATGGCTGTCGT |
| | | | CAGCTCGTGTCGTGAGATGTTG |
| | | | GGTTAAGTCCCGCAACGAGCGC |
| | | | AACCCCTACTCTTAGTTGCTAAT |
| | | | TAAAGAACTTTAAGAGAACAGCT |
| | | | AACAATAAGTTTAGAGGAAGGA |
| | | | GGGGATGACTTCAAGTCCTCAT |
| | | | GGCCCTTATGGGCAGGGCTTCA |
| | | | CACGTCATACAATGGTTAATACA |
| | | | AAAAGTTGCAATATCGTAAGATT |
| | | | GAGCTAATCTTTAAAATTAATCT |
| | | | TAGTTCGGATTGTACTCTGCAA |
| | | | CTCGAGTACATGAAGTTGGAAT |
| | | | CGCTAGTAATCGCGGATCAGCA |
| | | | TGCCGCGGTGAATAGTTTAACT |
| | | | GGTCTTGTACACACCGCCCGTC |
| | | | ACACCATGGAAATAAATCTTGTT |
| | | | TTAAATGAAGTAATATATTTTATC |
| | | | AAAACAGGTTTTGTAACCGGGG |
| | | | TGAAGTCGTAACA |
| | | | (SEQ ID NO: 20) |
| Zinderia insecticola CARI | spittlebug | bacteriocytes | ATATAAATAAGAGTTTGATCCTG |
| | Clastoptera | | GCTCAGATTGAACGCTAGCGGT |
| | arizonana | | ATGCTTTACACATGCAAGTCGA |
| | | | ACGACAATATTAAAGCTTGCTTT |
| | | | AATATAAAGTGGCGAACGGGTG |
| | | | AGTAATATATCAAAACGTACCTT |
| | | | AAAGTGGGGGATAACTAATTGA |
| | | | AAAATTAGATAATACCGCATATT |
| | | | AATCTTAGGATGAAAATAGGAAT |
| | | | AATATCTTATGCTTTTAGATCGG |
| | | | TTGATATCTGATTAGCTAGTTGG |
| | | | TAGGGTAAATGCTTACCAAGGC |
| | | | AATGATCAGTAGCTGGTTTTAG |
| | | | CGAATGATCAGCCACACTGGAA |
| | | | CTGAGACACGGTCCAGACTTCT |
| | | | ACGGAAGGCAGCAGTGGGGAA |
| | | | TATTGGACAATGGGAGAAATCC |
| | | | TGATCCAGCAATACCGCGTGAG |
| | | | TGATGAAGGCCTTAGGGTCGTA |
| | | | AAACTCTTTTGTTAGGAAAGAAA |
| | | | TAATTTTAAATAATATTTAAAATT |
| | | | GATGACGGTACCTAAAGAATAA |
| | | | GCACCGGCTAACTACGTGCCAG |
| | | | CAGCCGCGGTAATACGTAGGGT |
| | | | GCAAGCGTTAATCGGAATTATT |
| | | | GGGCGTAAAGAGTGCGTAGGC |
| | | | TGTTATATAAGATAGATGTGAAA |
| | | | TACTTAAGCTTAACTTAAGAACT |
| | | | GCATTTATTACTGTTTAACTAGA |
| | | | GTTTATTAGAGAGAAGTGGAATT |
| | | | TTATGTGTAGCAGTGAAATGCG |
| | | | TAGATATATAAAGGAATATCGAT |
| | | | GGCGAAGGCAGCTTCTTGGAAT |
| | | | AATACTGACGCTGAGGCACGAA |
| | | | AGCGTGGGGAGCAAACAGGATT |
| | | | AGATACCCTGGTAGTCCACGCC |
| | | | CTAAACTATGTCTACTAGTTATT |
| | | | AAATTAAAAATAAAATTTAGTAA |
| | | | CGTAGCTAACGCATTAAGTAGA |
| | | | CCGCCTGGGGAGTACGATCGC |
| | | | AAGATTAAAACTCAAAGGAATTG |
| | | | ACGGGGACCCGCACAAGCGGT |
| | | | GGATGATGTGGATTAATTCGAT |
| | | | GCAACACGAAAAACCTTACCTA |
| | | | CTCTTGACATGTTTGGAATTTTA |
| | | | AAGAAATTTAAAAGTGCTTGAAA |
| | | | AAGAACCAAAACACAGGTGCTG |
| | | | CATGGCTGTCGTCAGCTCGTGT |
| | | | CGTGAGATGTTGGGTTAAGTCC |
| | | | CGCAACGAGCGCAACCCTTGTT |
| | | | ATTATTTGCTAATAAAAAGAACT |
| | | | TTAATAAGACTGCCAATGACAAA |
| | | | TTGGAGGAAGGTGGGGATGAC |
| | | | GTCAAGTCCTCATGGCCCTTAT |
| | | | GAGTAGGGCTTCACACGTCATA |
| | | | CAATGATATATACAATGGGTAG |
| | | | CAAATTTGTGAAAATGAGCCAAT |
| | | | CCTTAAAGTATATCTTAGTTCGG |
| | | | ATTGTAGTCTGCAACTCGACTA |
| | | | CATGAAGTTGGAATCGCTAGTA |
| | | | ATCGCGGATCAGCATGCCGCG |
| | | | GTGAATACGTTCTCGGGTCTTG |
| | | | TACACACCGCCCGTCACACCAT |
| | | | GGAAGTGATTTTTACCAGAAATT |
| | | | ATTTGTTTAACCTTTATTGGAAA |
| | | | AAAATAATTAAGGTAGAATTCAT |
| | | | GACTGGGGTGAAGTCGTAACAA |
| | | | GGTAGCAGTATCGGAAGGTGC |
| | | | GGCTGGATTACATTTTAAAT |
| | | | (SEQ ID NO: 21) |
| Profftella armatura | Diaphorina citri, | bacteriomes | |
| | the Asian citrus | | |
| | psyllid | | |
| Alpha proteobacteria | | | |
| Hodgkinia | Cicada | bacteriome | AATGCTGGCGGCAGGCCTAACA |
| | Diceroprocta | | CATGCAAGTCGAGCGGACAACG |
| | semicincta | | TTCAAACGTTGTTAGCGGCGAA |
| | | | CGGGTGAGTAATACGTGAGAAT |
| | | | CTACCCATCCCAACGTGATAAC |
| | | | ATAGTCAACACCATGTCAATAAC |
| | | | GTATGATTCCTGCAACAGGTAA |
| | | | AGATTTTATCGGGGATGGATGA |
| | | | GCTCACGCTAGATTAGCTAGTT |
| | | | GGTGAGATAAAAGCCCACCAAG |
| | | | GCCAAGATCTATAGCTGGTCTG |
| | | | GAAGGATGGACAGCCACATTGG |
| | | | GACTGAGACAAGGCCCAACCCT |
| | | | CTAAGGAGGGCAGCAGTGAGG |
| | | | AATATTGGACAATGGGCGTAAG |
| | | | CCTGATCCAGCCATGCCGCATG |
| | | | AGTGATTGAAGGTCCAACGGAC |
| | | | TGTAAAACTCTTTTCTCCAGAGA |
| | | | TCATAAATGATAGTATCTGGTGA |
| | | | TATAAGCTCCGGCCAACTTCGT |
| | | | GCCAGCAGCCGCGGTAATACG |
| | | | AGGGGAGCGAGTATTGTTCGGT |
| | | | TTTATTGGGCGTAAAGGGTGTC |
| | | | CAGGTTGCTAAGTAAGTTAACA |
| | | | ACAAAATCTTGAGATTCAACCTC |
| | | | ATAACGTTCGGTTAATACTACTA |
| | | | AGCTCGAGCTTGGATAGAGACA |
| | | | AACGGAATTCCGAGTGTAGAGG |
| | | | TGAAATTCGTTGATACTTGGAG |
| | | | GAACACCAGAGGCGAAGGCGG |
| | | | TTTGTCATACCAAGCTGACACT |
| | | | GAAGACACGAAAGCATGGGGA |
| | | | GCAAACAGGATTAGATACCCTG |
| | | | GTAGTCCATGCCCTAAACGTTG |
| | | | AGTGCTAACAGTTCGATCAAGC |
| | | | CACATGCTATGATCCAGGATTG |
| | | | TACAGCTAACGCGTTAAGCACT |
| | | | CCGCCTGGGTATTACGACCGCA |
| | | | AGGTTAAAACTCAAAGGAATTG |
| | | | ACGGAGACCCGCACAAGCGGT |
| | | | GGAGCATGTGGTTTAATTCGAA |
| | | | GCTACACGAAGAACCTTACCAG |
| | | | CCCTTGACATACCATGGCCAAC |
| | | | CATCCTGGAAACAGGATGTTGT |
| | | | TCAAGTTAAACCCTTGAAATGCC |
| | | | AGGAACAGGTGCTGCATGGCTG |
| | | | TTGTCAGTTCGTGTCGTGAGAT |
| | | | GTATGGTTAAGTCCCAAAACGA |
| | | | ACACAACCCTCACCCATAGTTG |
| | | | CCATAAACACAATTGGGTTCTCT |
| | | | ATGGGTACTGCTAACGTAAGTT |
| | | | AGAGGAAGGTGAGGACCACAA |
| | | | CAAGTCATCATGGCCCTTATGG |
| | | | GCTGGGCCACACACATGCTACA |
| | | | ATGGTGGTTACAAAGAGCCGCA |
| | | | ACGTTGTGAGACCGAGCAAATC |
| | | | TCCAAAGACCATCTCAGTCCGG |
| | | | ATTGTACTCTGCAACCCGAGTA |
| | | | CATGAAGTAGGAATCGCTAGTA |
| | | | ATCGTGGATCAGCATGCCACGG |
| | | | TGAATACGTTCTCGGGTCTTGT |
| | | | ACACGCCGCCCGTCACACCATG |
| | | | GGAGCTTCGCTCCGATCGAAGT |
| | | | CAAGTTACCCTTGACCACATCTT |
| | | | GGCAAGTGACCGA |
| | | | (SEQ ID NO: 22) |
| Wolbachia sp. wPip | Mosquito | bacteriome | AAATTTGAGAGTTTGATCCTGG |
| | Culex | | CTCAGAATGAACGCTGGCGGCA |
| | quinquefasciatus | | GGCCTAACACATGCAAGTCGAA |
| | | | CGGAGTTATATTGTAGCTTGCTA |
| | | | TGGTATAACTTAGTGGCAGACG |
| | | | GGTGAGTAATGTATAGGAATCT |
| | | | ACCTAGTAGTACGGAATAATTGT |
| | | | TGGAAACGACAACTAATACCGT |
| | | | ATACGCCCTACGGGGGAAAAAT |
| | | | TTATTGCTATTAGATGAGCCTAT |
| | | | ATTAGATTAGCTAGTTGGTGGG |
| | | | GTAATAGCCTACCAAGGTAATG |
| | | | ATCTATAGCTGATCTGAGAGGA |
| | | | TGATCAGCCACACTGGAACTGA |
| | | | GATACGGTCCAGACTCCTACGG |
| | | | GAGGCAGCAGTGGGGAATATTG |
| | | | GACAATGGGCGAAAGCCTGATC |
| | | | CAGCCATGCCGCATGAGTGAAG |
| | | | AAGGCCTTTGGGTTGTAAAGCT |
| | | | CTTTTAGTGAGGAAGATAATGA |
| | | | CGGTACTCACAGAAGAAGTCCT |
| | | | GGCTAACTCCGTGCCAGCAGCC |
| | | | GCGGTAATACGGAGAGGGCTA |
| | | | GCGTTATTCGGAATTATTGGGC |
| | | | GTAAAGGGCGCGTAGGCTGGTT |
| | | | AATAAGTTAAAAGTGAAATCCCG |
| | | | AGGCTTAACCTTGGAATTGCTTT |
| | | | TAAAACTATTAATCTAGAGATTG |
| | | | AAAGAGGATAGAGGAATTCCTG |
| | | | ATGTAGAGGTAAAATTCGTAAAT |
| | | | ATTAGGAGGAACACCAGTGGCG |
| | | | AAGGCGTCTATCTGGTTCAAAT |
| | | | CTGACGCTGAAGCGCGAAGGC |
| | | | GTGGGGAGCAAACAGGATTAGA |
| | | | TACCCTGGTAGTCCACGCTGTA |
| | | | AACGATGAATGTTAAATATGGG |
| | | | GAGTTTACTTTCTGTATTACAGC |
| | | | TAACGCGTTAAACATTCCGCCT |
| | | | GGGGACTACGGTCGCAAGATTA |
| | | | AAACTCAAAGGAATTGACGGGG |
| | | | ACCCGCACAAGCGGTGGAGCA |
| | | | TGTGGTTTAATTCGATGCAACG |
| | | | CGAAAAACCTTACCACTTCTTGA |
| | | | CATGAAAATCATACCTATTCGAA |
| | | | GGGATAGGGTCGGTTCGGCCG |
| | | | GATTTTACACAAGTGTTGCATG |
| | | | GCTGTCGTCAGCTCGTGTCGTG |
| | | | AGATGTTGGGTTAAGTCCCGCA |
| | | | ACGAGCGCAACCCTCATCCTTA |
| | | | GTTGCCATCAGGTAATGCTGAG |
| | | | TACTTTAAGGAAACTGCCAGTG |
| | | | ATAAGCTGGAGGAAGGTGGGG |
| | | | ATGATGTCAAGTCATCATGGCC |
| | | | TTTATGGAGTGGGCTACACACG |
| | | | TGCTACAATGGTGTCTACAATG |
| | | | GGCTGCAAGGTGCGCAAGCCT |
| | | | AAGCTAATCCCTAAAAGACATCT |
| | | | CAGTTCGGATTGTACTCTGCAA |
| | | | CTCGAGTACATGAAGTTGGAAT |
| | | | CGCTAGTAATCGTGGATCAGCA |
| | | | TGCCACGGTGAATACGTTCTCG |
| | | | GGTCTTGTACACACTGCCCGTC |
| | | | ACGCCATGGGAATTGGTTTCAC |
| | | | TCGAAGCTAATGGCCTAACCGC |
| | | | AAGGAAGGAGTTATTTAAAGTG |
| | | | GGATCAGTGACTGGGGTGAAGT |
| | | | CGTAACAAGGTAGCAGTAGGGG |
| | | | AATCTGCAGCTGGATTACCTCC |
| | | | TTA |
| | | | (SEQ ID NO: 23) |
| Bacteroidetes | | | |
| Uzinura diaspidicola | armoured scale | bacteriocytes | AAAGGAGATATTCCAACCACAC |
| | insects | | CTTCCGGTACGGTTACCTTGTT |
| | | | ACGACTTAGCCCTAGTCATCAA |
| | | | GTTTACCTTAGGCAGACCACTG |
| | | | AAGGATTACTGACTTCAGGTAC |
| | | | CCCCGACTCCCATGGCTTGACG |
| | | | GGCGGTGTGTACAAGGTTCGAG |
| | | | AACATATTCACCGCGCCATTGC |
| | | | TGATGCGCGATTACTAGCGATT |
| | | | CCTGCTTCATAGAGTCGAATTG |
| | | | CAGACTCCAATCCGAACTGAGA |
| | | | CTGGTTTTAGAGATTAGCTCCT |
| | | | GATCACCCAGTGGCTGCCCTTT |
| | | | GTAACCAGCCATTGTAGCACGT |
| | | | GTGTAGCCCAAGGCATAGAGGC |
| | | | CATGATGATTTGACATCATCCCC |
| | | | ACCTTCCTCACAGTTTACACCG |
| | | | GCAGTTTTGTTAGAGTCCCCGG |
| | | | CTTTACCCGATGGCAACTAACA |
| | | | ATAGGGGTTGCGCTCGTTATAG |
| | | | GACTTAACCAAACACTTCACAG |
| | | | CACGAACTGAAGACAACCATGC |
| | | | AGCACCTTGTAATACGTCGTATA |
| | | | GACTAAGCTGTTTCCAGCTTATT |
| | | | CGTAATACATTTAAGCCTTGGTA |
| | | | AGGTTCCTCGCGTATCATCGAA |
| | | | TTAAACCACATGCTCCACCGCT |
| | | | TGTGCGAACCCCCGTCAATTCC |
| | | | TTTGAGTTTCAATCTTGCGACTG |
| | | | TACTTCCCAGGTGGATCACTTAT |
| | | | CGCTTTCGCTAAGCCACTGAAT |
| | | | ATCGTTTTTCCAATAGCTAGTGA |
| | | | TCATCGTTTAGGGCGTGGACTA |
| | | | CCAGGGTATCTAATCCTGTTTG |
| | | | CTCCCCACGCTTTCGTGCACTG |
| | | | AGCGTCAGTAAAGATTTAGCAA |
| | | | CCTGCCTTCGCTATCGGTGTTC |
| | | | TGTATGATATCTATGCATTTCAC |
| | | | CGCTACACCATACATTCCAGAT |
| | | | GCTCCAATCTTACTCAAGTTTAC |
| | | | CAGTATCAATAGCAATTTTACAG |
| | | | TTAAGCTGTAAGCTTTCACTACT |
| | | | GACTTAATAAACAGCCTACACA |
| | | | CCCTTTAAACCCAATAAATCCGA |
| | | | ATAACGCTTGTGTCATCCGTATT |
| | | | GCCGCGGCTGCTGGCACGGAA |
| | | | TTAGCCGACACTTATTCGTATAG |
| | | | TACCTTCAATCTCCTATCACGTA |
| | | | AGATATTTTATTTCTATACAAAA |
| | | | GCAGTTTACAACCTAAAAGACC |
| | | | TTCATCCTGCACGCGACGTAGC |
| | | | TGGTTCAGAGTTTCCTCCATTGA |
| | | | CCAATATTCCTCACTGCTGCCT |
| | | | CCCGTAGGAGTCTGGTCCGTGT |
| | | | CTCAGTACCAGTGTGGAGGTAC |
| | | | ACCCTCTTAGGCCCCCTACTGA |
| | | | TCATAGTCTTGGTAGAGCCATTA |
| | | | CCTCACCAACTAACTAATCAAAC |
| | | | GCAGGCTCATCTTTTGCCACCT |
| | | | AAGTTTTAATAAAGGCTCCATGC |
| | | | AGAAACTTTATATTATGGGGGAT |
| | | | TAATCAGAATTTCTTCTGGCTAT |
| | | | ACCCCAGCAAAAGGTAGATTGC |
| | | | ATACGTGTTACTCACCCATTCG |
| | | | CCGGTCGCCGACAAATTAAAAA |
| | | | TTTTTCGATGCCCCTCGACTTG |
| | | | CATGTGTTAAGCTCGCCGCTAG |
| | | | CGTTAATTCTGAGCCAGGATCA |
| | | | AACTCTTCGTTGTAG |
| | | | (SEQ ID NO: 24) |
| Sulcia muelleri | Blue-Green | bacteriocytes | CTCAGGATAAACGCTAGCGGAG |
| | Sharpshooter | | GGCTTAACACATGCAAGTCGAG |
| | and several other | | GGGCAGCAAAAATAATTATTTTT |
| | leafhopper | | GGCGACCGGCAAACGGGTGAG |
| | species | | TAATACATACGTAACTTTCCTTA |
| | | | TGCTGAGGAATAGCCTGAGGAA |
| | | | ACTTGGATTAATACCTCATAATA |
| | | | CAATTTTTTAGAAAGAAAAATTG |
| | | | TTAAAGTTTTATTATGGCATAAG |
| | | | ATAGGCGTATGTCCAATTAGTTA |
| | | | GTTGGTAAGGTAATGGCTTACC |
| | | | AAGACGATGATTGGTAGGGGGC |
| | | | CTGAGAGGGGCGTTCCCCCAC |
| | | | ATTGGTACTGAGACACGGACCA |
| | | | AACTTCTACGGAAGGCTGCAGT |
| | | | GAGGAATATTGGTCAATGGAGG |
| | | | AAACTCTGAACCAGCCACTCCG |
| | | | CGTGCAGGATGAAAGAAAGCCT |
| | | | TATTGGTTGTAAACTGCTTTTGT |
| | | | ATATGAATAAAAAATTCTAATTAT |
| | | | AGAAATAATTGAAGGTAATATAC |
| | | | GAATAAGTATCGACTAACTCTGT |
| | | | GCCAGCAGTCGCGGTAAGACA |
| | | | GAGGATACAAGCGTTATCCGGA |
| | | | TTTATTGGGTTTAAAGGGTGCG |
| | | | TAGGCGGTTTTTAAAGTCAGTA |
| | | | GTGAAATCTTAAAGCTTAACTTT |
| | | | AAAAGTGCTATTGATACTGAAAA |
| | | | ACTAGAGTAAGGTTGGAGTAAC |
| | | | TGGAATGTGTGGTGTAGCGGTG |
| | | | AAATGCATAGATATCACACAGAA |
| | | | CACCGATAGCGAAAGCAAGTTA |
| | | | CTAACCCTATACTGACGCTGAG |
| | | | TCACGAAAGCATGGGGAGCAAA |
| | | | CAGGATTAGATACCCTGGTAGT |
| | | | CCATGCCGTAAACGATGATCAC |
| | | | TAACTATTGGGTTTTATACGTTG |
| | | | TAATTCAGTGGTGAAGCGAAAG |
| | | | TGTTAAGTGATCCACCTGAGGA |
| | | | GTACGACCGCAAGGTTGAAACT |
| | | | CAAAGGAATTGACGGGGGCCC |
| | | | GCACAATCGGTGGAGCATGTGG |
| | | | TTTAATTCGATGATACACGAGGA |
| | | | ACCTTACCAAGACTTAAATGTAC |
| | | | TACGAATAAATTGGAAACAATTT |
| | | | AGTCAAGCGACGGAGTACAAGG |
| | | | TGCTGCATGGTTGTCGTCAGCT |
| | | | CGTGCCGTGAGGTGTAAGGTTA |
| | | | AGTCCTTTAAACGAGCGCAACC |
| | | | CTTATTATTAGTTGCCATCGAGT |
| | | | AATGTCAGGGGACTCTAATAAG |
| | | | ACTGCCGGCGCAAGCCGAGAG |
| | | | GAAGGTGGGGATGACGTCAAAT |
| | | | CATCACGGCCCTTACGTCTTGG |
| | | | GCCACACACGTGCTACAATGAT |
| | | | CGGTACAAAAGGGAGCGACTG |
| | | | GGTGACCAGGAGCAAATCCAGA |
| | | | AAGCCGATCTAAGTTCGGATTG |
| | | | GAGTCTGAAACTCGACTCCATG |
| | | | AAGCTGGAATCGCTAGTAATCG |
| | | | TGCATCAGCCATGGCACGGTGA |
| | | | ATATGTTCCCGGGCCTTGTACA |
| | | | CACCGCCCGTCAAGCCATGGAA |
| | | | GTTGGAAGTACCTAAAGTTGGT |
| | | | TCGCTACCTAAGGTAAGTCTAAT |
| | | | AACTGGGGCTAAGTCGTAACAA |
| | | | GGTA |
| | | | (SEQ ID NO: 25) |
| Yeast like | | | |
| Symbiotaphrina buchneri | Anobiid beetles | mycetome | AGATTAAGCCATGCAAGTCTAA |
| voucher JCM9740 | Stegobium | between the | GTATAAGNAATCTATACNGTGAA |
| | paniceum | foregut and | ACTGCGAATGGCTCATTAAATC |
| | | midgut | AGTTATCGTTTATTTGATAGTAC |
| | | | CTTACTACATGGATAACCGTGG |
| | | | TAATTCTAGAGCTAATACATGCT |
| | | | AAAAACCCCGACTTCGGAAGGG |
| | | | GTGTATTTATTAGATAAAAAACC |
| | | | AATGCCCTTCGGGGCTCCTTGG |
| | | | TGATTCATGATAACTTAACGAAT |
| | | | CGCATGGCCTTGCGCCGGCGA |
| | | | TGGTTCATTCAAATTTCTGCCCT |
| | | | ATCAACTTTCGATGGTAGGATA |
| | | | GTGGCCTACCATGGTTTTAACG |
| | | | GGTAACGGGGAATTAGGGTTCG |
| | | | ATTCCGGAGAGGGAGCCTGAG |
| | | | AAACGGCTACCACATCCAAGGA |
| | | | AGGCAGCAGGCGCGCAAATTAC |
| | | | CCAATCCCGACACGGGGAGGT |
| | | | AGTGACAATAAATACTGATACAG |
| | | | GGCTCTTTTGGGTCTTGTAATTG |
| | | | GAATGAGTACAATTTAAATCCCT |
| | | | TAACGAGGAACAATTGGAGGGC |
| | | | AAGTCTGGTGCCAGCAGCCGC |
| | | | GGTAATTCCAGCTCCAATAGCG |
| | | | TATATTAAAGTTGTTGCAGTTAA |
| | | | AAAGCTCGTAGTTGAACCTTGG |
| | | | GCCTGGCTGGCCGGTCCGCCT |
| | | | AACCGCGTGTACTGGTCCGGCC |
| | | | GGGCCTTTCCTTCTGGGGAGCC |
| | | | GCATGCCCTTCACTGGGTGTGT |
| | | | CGGGGAACCAGGACTTTTACTT |
| | | | TGAAAAAATTAGAGTGTTCAAAG |
| | | | CAGGCCTATGCTCGAATACATT |
| | | | AGCATGGAATAATAGAATAGGA |
| | | | CGTGCGGTTCTATTTTGTTGGTT |
| | | | TCTAGGACCGCCGTAATGATTA |
| | | | ATAGGGATAGTCGGGGGCATCA |
| | | | GTATTCAATTGTCAGAGGTGAA |
| | | | ATTCTTGGATTTATTGAAGACTA |
| | | | ACTACTGCGAAAGCATTTGCCA |
| | | | AGGATGTTTTCATTAATCAGTGA |
| | | | ACGAAAGTTAGGGGATCGAAGA |
| | | | CGATCAGATACCGTCGTAGTCT |
| | | | TAACCATAAACTATGCCGACTA |
| | | | GGGATCGGGCGATGTTATTATT |
| | | | TTGACTCGCTCGGCACCTTACG |
| | | | AGAAATCAAAGTCTTTGGGTTCT |
| | | | GGGGGGAGTATGGTCGCAAGG |
| | | | CTGAAACTTAAAGAAATTGACG |
| | | | GAAGGGCACCACCAGGAGTGG |
| | | | AGCCTGCGGCTTAATTTGACTC |
| | | | AACACGGGGAAACTCACCAGGT |
| | | | CCAGACACATTAAGGATTGACA |
| | | | GATTGAGAGCTCTTTCTTGATTA |
| | | | TGTGGGTGGTGGTGCATGGCC |
| | | | GTTCTTAGTTGGTGGAGTGATTT |
| | | | GTCTGCTTAATTGCGATAACGA |
| | | | ACGAGACCTTAACCTGCTAAAT |
| | | | AGCCCGGTCCGCTTTGGCGGG |
| | | | CCGCTGGCTTCTTAGAGGGACT |
| | | | ATCGGCTCAAGCCGATGGAAGT |
| | | | TTGAGGCAATAACAGGTCTGTG |
| | | | ATGCCCTTAGATGTTCTGGGCC |
| | | | GCACGCGCGCTACACTGACAGA |
| | | | GCCAACGAGTAAATCACCTTGG |
| | | | CCGGAAGGTCTGGGTAATCTTG |
| | | | TTAAACTCTGTCGTGCTGGGGA |
| | | | TAGAGCATTGCAATTATTGCTCT |
| | | | TCAACGAGGAATTCCTAGTAAG |
| | | | CGCAAGTCATCAGCTTGCGCTG |
| | | | ATTACGTCCCTGCCCTTTGTACA |
| | | | CACCGCCCGTCGCTACTACCGA |
| | | | TTGAATGGCTCAGTGAGGCCTT |
| | | | CGGACTGGCACAGGGACGTTG |
| | | | GCAACGACGACCCAGTGCCGG |
| | | | AAAGTTGGTCAAACTTGGTCATT |
| | | | TAGAGGAAGTAAAAGTCGTAAC |
| | | | AAGGTTTCCGTAGGTGAACCTG |
| | | | CGGAAGGATCATTA |
| | | | (SEQ ID NO: 26) |
| Symbiotaphrina kochii | Anobiid beetles | mycetome | TACCTGGTTGATTCTGCCAGTA |
| voucher CBS 589.63 | Lasioderma | | GTCATATGCTTGTCTCAAAGATT |
| | serricorne | | AAGCCATGCAAGTCTAAGTATA |
| | | | AGCAATCTATACGGTGAAACTG |
| | | | CGAATGGCTCATTAAATCAGTTA |
| | | | TCGTTTATTTGATAGTACCTTAC |
| | | | TACATGGATAACCGTGGTAATT |
| | | | CTAGAGCTAATACATGCTAAAAA |
| | | | CCTCGACTTCGGAAGGGGTGTA |
| | | | TTTATTAGATAAAAAACCAATGC |
| | | | CCTTCGGGGCTCCTTGGTGATT |
| | | | CATGATAACTTAACGAATCGCAT |
| | | | GGCCTTGCGCCGGCGATGGTT |
| | | | CATTCAAATTTCTGCCCTATCAA |
| | | | CTTTCGATGGTAGGATAGTGGC |
| | | | CTACCATGGTTTCAACGGGTAA |
| | | | CGGGGAATTAGGGTTCGATTCC |
| | | | GGAGAGGGAGCCTGAGAAACG |
| | | | GCTACCACATCCAAGGAAGGCA |
| | | | GCAGGCGCGCAAATTACCCAAT |
| | | | CCCGACACGGGGAGGTAGTGA |
| | | | CAATAAATACTGATACAGGGCT |
| | | | CTTTTGGGTCTTGTAATTGGAAT |
| | | | GAGTACAATTTAAATCCCTTAAC |
| | | | GAGGAACAATTGGAGGGCAAGT |
| | | | CTGGTGCCAGCAGCCGCGGTA |
| | | | ATTCCAGCTCCAATAGCGTATAT |
| | | | TAAAGTTGTTGCAGTTAAAAAGC |
| | | | TCGTAGTTGAACCTTGGGCCTG |
| | | | GCTGGCCGGTCCGCCTAACCG |
| | | | CGTGTACTGGTCCGGCCGGGC |
| | | | CTTTCCTTCTGGGGAGCCGCAT |
| | | | GCCCTTCACTGGGTGTGTCGGG |
| | | | GAACCAGGACTTTTACTTTGAAA |
| | | | AAATTAGAGTGTTCAAAGCAGG |
| | | | CCTATGCTCGAATACATTAGCAT |
| | | | GGAATAATAGAATAGGACGTGT |
| | | | GGTTCTATTTTGTTGGTTTCTAG |
| | | | GACCGCCGTAATGATTAATAGG |
| | | | GATAGTCGGGGGCATCAGTATT |
| | | | CAATTGTCAGAGGTGAAATTCTT |
| | | | GGATTTATTGAAGACTAACTACT |
| | | | GCGAAAGCATTTGCCAAGGATG |
| | | | TTTTCATTAATCAGTGAACGAAA |
| | | | GTTAGGGGATCGAAGACGATCA |
| | | | GATACCGTCGTAGTCTTAACCA |
| | | | TAAACTATGCCGACTAGGGATC |
| | | | GGGCGATGTTATTATTTTGACTC |
| | | | GCTCGGCACCTTACGAGAAATC |
| | | | AAAGTCTTTGGGTTCTGGGGGG |
| | | | AGTATGGTCGCAAGGCTGAAAC |
| | | | TTAAAGAAATTGACGGAAGGGC |
| | | | ACCACCAGGAGTGGAGCCTGC |
| | | | GGCTTAATTTGACTCAACACGG |
| | | | GGAAACTCACCAGGTCCAGACA |
| | | | CATTAAGGATTGACAGATTGAG |
| | | | AGCTCTTTCTTGATTATGTGGGT |
| | | | GGTGGTGCATGGCCGTTCTTAG |
| | | | TTGGTGGAGTGATTTGTCTGCT |
| | | | TAATTGCGATAACGAACGAGAC |
| | | | CTTAACCTGCTAAATAGCCCGG |
| | | | TCCGCTTTGGCGGGCCGCTGG |
| | | | CTTCTTAGAGGGACTATCGGCT |
| | | | CAAGCCGATGGAAGTTTGAGGC |
| | | | AATAACAGGTCTGTGATGCCCT |
| | | | TAGATGTTCTGGGCCGCACGCG |
| | | | CGCTACACTGACAGAGCCAACG |
| | | | AGTACATCACCTTGGCCGGAAG |
| | | | GTCTGGGTAATCTTGTTAAACTC |
| | | | TGTCGTGCTGGGGATAGAGCAT |
| | | | TGCAATTATTGCTCTTCAACGAG |
| | | | GAATTCCTAGTAAGCGCAAGTC |
| | | | ATCAGCTTGCGCTGATTACGTC |
| | | | CCTGCCCTTTGTACACACCGCC |
| | | | CGTCGCTACTACCGATTGAATG |
| | | | GCTCAGTGAGGCCTTCGGACTG |
| | | | GCACAGGGACGTTGGCAACGA |
| | | | CGACCCAGTGCCGGAAAGTTCG |
| | | | TCAAACTTGGTCATTTAGAGGAA |
| | | | GNNNAAGTCGTAACAAGGTTTC |
| | | | CGTAGGTGAACCTGCGGAAGG |
| | | | ATCATTA |
| | | | (SEQ ID NO: 27) |
| Primary extracelullar | | | |
| symbiont | Host | Location | 16 rRNA |
| fenitrothion-degrading | | | |
| bacteria | | | |
| Burkholderia sp. SFA1 | Riptortus | Gut | AGTTTGATCCTGGCTCAGATTG |
| | pedestris | | AACGCTGGCGGCATGCCTTACA |
| | | | CATGCAAGTCGAACGGCAGCAC |
| | | | GGGGGCAACCCTGGTGGCGAG |
| | | | TGGCGAACGGGTGAGTAATACA |
| | | | TCGGAACGTGTCCTGTAGTGGG |
| | | | GGATAGCCCGGCGAAAGCCGG |
| | | | ATTAATACCGCATACGACCTAA |
| | | | GGGAGAAAGCGGGGGATCTTC |
| | | | GGACCTCGCGCTATAGGGGCG |
| | | | GCCGATGGCAGATTAGCTAGTT |
| | | | GGTGGGGTAAAGGCCTACCAA |
| | | | GGCGACGATCTGTAGCTGGTCT |
| | | | GAGAGGACGACCAGCCACACT |
| | | | GGGACTGAGACACGGCCCAGA |
| | | | CTCCTACGGGAGGCAGCAGTG |
| | | | GGGAATTTTGGACAATGGGGGC |
| | | | AACCCTGATCCAGCAATGCCGC |
| | | | GTGTGTGAAGAAGGCTTCGGGT |
| | | | TGTAAAGCACTTTTGTCCGGAA |
| | | | AGAAAACTTCGTCCCTAATATG |
| | | | GATGGAGGATGACGGTACCGG |
| | | | AAGAATAAGCACCGGCTAACTA |
| | | | CGTGCCAGCAGCCGCGGTAATA |
| | | | CGTAGGGTGCGAGCGTTAATCG |
| | | | GAATTACTGGGCGTAAAGCGTG |
| | | | CGCAGGCGGTCTGTTAAGACCG |
| | | | ATGTGAAATCCCCGGGCTTAAC |
| | | | CTGGGAACTGCATTGGTGACTG |
| | | | GCAGGCTTTGAGTGTGGCAGAG |
| | | | GGGGGTAGAATTCCACGTGTAG |
| | | | CAGTGAAATGCGTAGAGATGTG |
| | | | GAGGAATACCGATGGCGAAGG |
| | | | CAGCCCCCTGGGCCAACTACTG |
| | | | ACGCTCATGCACGAAAGCGTGG |
| | | | GGAGCAAACAGGATTAGATACC |
| | | | CTGGTAGTCCACGCCCTAAACG |
| | | | ATGTCAACTAGTTGTTGGGGAT |
| | | | TCATTTCCTTAGTAACGTAGCTA |
| | | | ACGCGTGAAGTTGACCGCCTGG |
| | | | GGAGTACGGTCGCAAGATTAAA |
| | | | ACTCAAAGGAATTGACGGGGAC |
| | | | CCGCACAAGCGGTGGATGATGT |
| | | | GGATTAATTCGATGCAACGCGA |
| | | | AAAACCTTACCTACCCTTGACAT |
| | | | GGTCGGAACCCTGCTGAAAGGT |
| | | | GGGGGTGCTCGAAAGAGAACC |
| | | | GGCGCACAGGTGCTGCATGGC |
| | | | TGTCGTCAGCTCGTGTCGTGAG |
| | | | ATGTTGGGTTAAGTCCCGCAAC |
| | | | GAGCGCAACCCTTGTCCTTAGT |
| | | | TGCTACGCAAGAGCACTCTAAG |
| | | | GAGACTGCCGGTGACAAACCG |
| | | | GAGGAAGGTGGGGATGACGTC |
| | | | AAGTCCTCATGGCCCTTATGGG |
| | | | TAGGGCTTCACACGTCATACAA |
| | | | TGGTCGGAACAGAGGGTTGCCA |
| | | | AGCCGCGAGGTGGAGCCAATC |
| | | | CCAGAAAACCGATCGTAGTCCG |
| | | | GATCGCAGTCTGCAACTCGACT |
| | | | GCGTGAAGCTGGAATCGCTAGT |
| | | | AATCGCGGATCAGCATGCCGCG |
| | | | GTGAATACGTTCCCGGGTCTTG |
| | | | TACACACCGCCCGTCACACCAT |
| | | | GGGAGTGGGTTTCACCAGAAGT |
| | | | AGGTAGCCTAACCGCAAGGAG |
| | | | GGCGCTTACCACGGTGGGATTC |
| | | | ATGACTGGGGTGAAGTCGTAAC |
| | | | AAGGTAGC |
| | | | (SEQ ID NO: 28) |
| Burkholderia sp. KM-A | Riptortus | Gut | GCAACCCTGGTGGCGAGTGGC |
| | pedestris | | GAACGGGTGAGTAATACATCGG |
| | | | AACGTGTCCTGTAGTGGGGGAT |
| | | | AGCCCGGCGAAAGCCGGATTAA |
| | | | TACCGCATACGATCTACGGAAG |
| | | | AAAGCGGGGGATCCTTCGGGA |
| | | | CCTCGCGCTATAGGGGCGGCC |
| | | | GATGGCAGATTAGCTAGTTGGT |
| | | | GGGGTAAAGGCCTACCAAGGC |
| | | | GACGATCTGTAGCTGGTCTGAG |
| | | | AGGACGACCAGCCACACTGGG |
| | | | ACTGAGACACGGCCCAGACTCC |
| | | | TACGGGAGGCAGCAGTGGGGA |
| | | | ATTTTGGACAATGGGGGCAACC |
| | | | CTGATCCAGCAATGCCGCGTGT |
| | | | GTGAAGAAGGCCTTCGGGTTGT |
| | | | AAAGCACTTTTGTCCGGAAAGA |
| | | | AAACGTCTTGGTTAATACCTGA |
| | | | GGCGGATGACGGTACCGGAAG |
| | | | AATAAGCACCGGCTAACTACGT |
| | | | GCCAGCAGCCGCGGTAATACGT |
| | | | AGGGTGCGAGCGTTAATCGGAA |
| | | | TTACTGGGCGTAAAGCGTGCGC |
| | | | AGGCGGTCTGTTAAGACCGATG |
| | | | TGAAATCCCCGGGCTTAACCTG |
| | | | GGAACTGCATTGGTGACTGGCA |
| | | | GGCTTTGAGTGTGGCAGAGGG |
| | | | GGGTAGAATTCCACGTGTAGCA |
| | | | GTGAAATGCGTAGAGATGTGGA |
| | | | GGAATACCGATGGCGAAGGCA |
| | | | GCCCCCTGGGCCAACACTGAC |
| | | | GCTCATGCACGAAAGCGTGGG |
| | | | GAGCAAACAGGATTAGATACCC |
| | | | TGGTAGTCCACGCCCTAAACGA |
| | | | TGTCAACTAGTTGTTGGGGATT |
| | | | CATTTCCTTAGTAACGTAGCTAA |
| | | | CGCGTGAAGTTGACCGCCTGG |
| | | | GGAGTACGGTCGCAAGATTAAA |
| | | | ACTCAAAGGAATTGACGGGGAC |
| | | | CCGCACAAGCGGTGGATGATGT |
| | | | GGATTAATTCGATGCAACGCGA |
| | | | AAAACCTTACCTACCCTTGACAT |
| | | | GGTCGGAAGTCTGCTGAGAGGT |
| | | | GGACGTGCTCGAAAGAGAACC |
| | | | GGCGCACAGGTGCTGCATGGC |
| | | | TGTCGTCAGCTCGTGTCGTGAG |
| | | | ATGTTGGGTTAAGTCCCGCAAC |
| | | | GAGCGCAACCCTTGTCCTTAGT |
| | | | TGCTACGCAAGAGCACTCTAAG |
| | | | GAGACTGCCGGTGACAAACCG |
| | | | GAGGAAGGTGGGGATGACGTC |
| | | | AAGTCCTCATGGCCCTTATGGG |
| | | | TAGGGCTTCACACGTCATACAA |
| | | | TGGTCGGAACAGAGGGTTGCCA |
| | | | AGCCGCGAGGTGGAGCCAATC |
| | | | CCAGAAAACCGATCGTAGTCCG |
| | | | GATCGCAGTCTGCAACTCGACT |
| | | | GCGTGAAGCTGGAATCGCTAG |
| | | | TAATCGCGGATCAGCATGCCGC |
| | | | GGTGAATACGTTCCCGGGTCTT |
| | | | GTACACACCGCCCGTCACACCA |
| | | | TGGGAGTGGGTTTCACCAGAAG |
| | | | TAGGTAGCCTAACCGCAAGGAG |
| | | | GGCGCTTACCACGGTGGGATTC |
| | | | ATGACTGGGGTGAAGT |
| | | | (SEQ ID NO: 29) |
| Burkholderia sp. KM-G | Riptortus | Gut | GCAACCCTGGTGGCGAGTGGC |
| | pedestris | | GAACGGGTGAGTAATACATCGG |
| | | | AACGTGTCCTGTAGTGGGGGAT |
| | | | AGCCCGGCGAAAGCCGGATTAA |
| | | | TACCGCATACGACCTAAGGGAG |
| | | | AAAGCGGGGGATCTTCGGACCT |
| | | | CGCGCTATAGGGGCGGCCGAT |
| | | | GGCAGATTAGCTAGTTGGTGGG |
| | | | GTAAAGGCCTACCAAGGCGACG |
| | | | ATCTGTAGCTGGTCTGAGAGGA |
| | | | CGACCAGCCACACTGGGACTGA |
| | | | GACACGGCCCAGACTCCTACG |
| | | | GGAGGCAGCAGTGGGGAATTTT |
| | | | GGACAATGGGGGCAACCCTGAT |
| | | | CCAGCAATGCCGCGTGTGTGAA |
| | | | GAAGGCCTTCGGGTTGTAAAGC |
| | | | ACTTTTGTCCGGAAAGAAAACTT |
| | | | CGAGGTTAATACCCTTGGAGGA |
| | | | TGACGGTACCGGAAGAATAAGC |
| | | | ACCGGCTAACTACGTGCCAGCA |
| | | | GCCGCGGTAATACGTAGGGTG |
| | | | CGAGCGTTAATCGGAATTACTG |
| | | | GGCGTAAAGCGTGCGCAGGCG |
| | | | GTCTGTTAAGACCGATGTGAAA |
| | | | TCCCCGGGCTTAACCTGGGAAC |
| | | | TGCATTGGTGACTGGCAGGCTT |
| | | | TGAGTGTGGCAGAGGGGGGTA |
| | | | GAATTCCACGTGTAGCAGTGAA |
| | | | ATGCGTAGAGATGTGGAGGAAT |
| | | | ACCGATGGCGAAGGCAGCCCC |
| | | | CTGGGCCAACACTGACGCTCAT |
| | | | GCACGAAAGCGTGGGGAGCAA |
| | | | ACAGGATTAGATACCCTGGTAG |
| | | | TCCACGCCCTAAACGATGTCAA |
| | | | CTAGTTGTTGGGGATTCATTTCC |
| | | | TTAGTAACGTAGCTAACGCGTG |
| | | | AAGTTGACCGCCTGGGGAGTAC |
| | | | GGTCGCAAGATTAAAACTCAAA |
| | | | GGAATTGACGGGGACCCGCAC |
| | | | AAGCGGTGGATGATGTGGATTA |
| | | | ATTCGATGCAACGCGAAAAACC |
| | | | TTACCTACCCTTGACATGGTCG |
| | | | GAAGTCTGCTGAGAGGTGGAC |
| | | | GTGCTCGAAAGAGAACCGGCG |
| | | | CACAGGTGCTGCATGGCTGTC |
| | | | GTCAGCTCGTGTCGTGAGATGT |
| | | | TGGGTTAAGTCCCGCAACGAGC |
| | | | GCAACCCTTGTCCTTAGTTGCT |
| | | | ACGCAAGAGCACTCTAAGGAGA |
| | | | CTGCCGGTGACAAACCGGAGG |
| | | | AAGGTGGGGATGACGTCAAGTC |
| | | | CTCATGGCCCTTATGGGTAGGG |
| | | | CTTCACACGTCATACAATGGTC |
| | | | GGAACAGAGGGTTGCCAAGCC |
| | | | GCGAGGTGGAGCCAATCCCAG |
| | | | AAAACCGATCGTAGTCCGGATC |
| | | | GCAGTCTGCAACTCGACTGCGT |
| | | | GAAGCTGGAATCGCTAGTAATC |
| | | | GCGGATCAGCATGCCGCGGTG |
| | | | AATACGTTCCCGGGTCTTGTAC |
| | | | ACACCGCCCGTCACACCATGGG |
| | | | AGTGGGTTTCACCAGAAGTAGG |
| | | | TAGCCTAACCTGCAAAGGAGGG |
| | | | CGCTTACCACG |
| | | | (SEQ ID NO: 30) |
| Snodgrassella alvi | Honeybee (Apis | Ileum | GAGAGTTTGATCCTGGCTCAGA |
| | mellifera) and | | TTGAACGCTGGCGGCATGCCTT |
| | Bombus spp. | | ACACATGCAAGTCGAACGGCAG |
| | | | CACGGAGAGCTTGCTCTCTGGT |
| | | | GGCGAGTGGCGAACGGGTGAG |
| | | | TAATGCATCGGAACGTACCGAG |
| | | | TAATGGGGGATAACTGTCCGAA |
| | | | AGGATGGCTAATACCGCATACG |
| | | | CCCTGAGGGGGAAAGCGGGGG |
| | | | ATCGAAAGACCTCGCGTTATTT |
| | | | GAGCGGCCGATGTTGGATTAGC |
| | | | TAGTTGGTGGGGTAAAGGCCTA |
| | | | CCAAGGCGACGATCCATAGCG |
| | | | GGTCTGAGAGGATGATCCGCCA |
| | | | CATTGGGACTGAGACACGGCCC |
| | | | AAACTCCTACGGGAGGCAGCAG |
| | | | TGGGGAATTTTGGACAATGGGG |
| | | | GGAACCCTGATCCAGCCATGCC |
| | | | GCGTGTCTGAAGAAGGCCTTCG |
| | | | GGTTGTAAAGGACTTTTGTTAG |
| | | | GGAAGAAAAGCCGGGTGTTAAT |
| | | | ACCATCTGGTGCTGACGGTACC |
| | | | TAAAGAATAAGCACCGGCTAAC |
| | | | TACGTGCCAGCAGCCGCGGTAA |
| | | | TACGTAGGGTGCGAGCGTTAAT |
| | | | CGGAATTACTGGGCGTAAAGCG |
| | | | AGCGCAGACGGTTAATTAAGTC |
| | | | AGATGTGAAATCCCCGAGCTCA |
| | | | ACTTGGGACGTGCATTTGAAAC |
| | | | TGGTTAACTAGAGTGTGTCAGA |
| | | | GGGAGGTAGAATTCCACGTGTA |
| | | | GCAGTGAAATGCGTAGAGATGT |
| | | | GGAGGAATACCGATGGCGAAG |
| | | | GCAGCCTCCTGGGATAACACTG |
| | | | ACGTTCATGCTCGAAAGCGTGG |
| | | | GTAGCAAACAGGATTAGATACC |
| | | | CTGGTAGTCCACGCCCTAAACG |
| | | | ATGACAATTAGCTGTTGGGACA |
| | | | CTAGATGTCTTAGTAGCGAAGC |
| | | | TAACGCGTGAAATTGTCCGCCT |
| | | | GGGGAGTACGGTCGCAAGATTA |
| | | | AAACTCAAAGGAATTGACGGGG |
| | | | ACCCGCACAAGCGGTGGATGAT |
| | | | GTGGATTAATTCGATGCAACGC |
| | | | GAAGAACCTTACCTGGTCTTGA |
| | | | CATGTACGGAATCTCTTAGAGA |
| | | | TAGGAGAGTGCCTTCGGGAACC |
| | | | GTAACACAGGTGCTGCATGGCT |
| | | | GTCGTCAGCTCGTGTCGTGAGA |
| | | | TGTTGGGTTAAGTCCCGCAACG |
| | | | AGCGCAACCCTTGTCATTAGTT |
| | | | GCCATCATTAAGTTGGGCACTC |
| | | | TAATGAGACTGCCGGTGACAAA |
| | | | CCGGAGGAAGGTGGGGATGAC |
| | | | GTCAAGTCCTCATGGCCCTTAT |
| | | | GACCAGGGCTTCACACGTCATA |
| | | | CAATGGTCGGTACAGAGGGTAG |
| | | | CGAAGCCGCGAGGTGAAGCCA |
| | | | ATCTCAGAAAGCCGATCGTAGT |
| | | | CCGGATTGCACTCTGCAACTCG |
| | | | AGTGCATGAAGTCGGAATCGCT |
| | | | AGTAATCGCAGGTCAGCATACT |
| | | | GCGGTGAATACGTTCCCGGGTC |
| | | | TTGTACACACCGCCCGTCACAC |
| | | | CATGGGAGTGGGGGATACCAG |
| | | | AATTGGGTAGACTAACCGCAAG |
| | | | GAGGTCGCTTAACACGGTATGC |
| | | | TTCATGACTGGGGTGAAGTCGT |
| | | | AACAAGGTAGCCGTAG |
| | | | (SEQ ID NO: 33) |
| Gilliamella apicola | honeybee (Apis | Ileum | TTAAATTGAAGAGTTTGATCATG |
| | mellifera) and | | GCTCAGATTGAACGCTGGCGGC |
| | Bombus spp. | | AGGCTTAACACATGCAAGTCGA |
| | | | ACGGTAACATGAGTGCTTGCAC |
| | | | TTGATGACGAGTGGCGGACGG |
| | | | GTGAGTAAAGTATGGGGATCTG |
| | | | CCGAATGGAGGGGGACAACAG |
| | | | TTGGAAACGACTGCTAATACCG |
| | | | CATAAAGTTGAGAGACCAAAGC |
| | | | ATGGGACCTTCGGGCCATGCG |
| | | | CCATTTGATGAACCCATATGGG |
| | | | ATTAGCTAGTTGGTAGGGTAAT |
| | | | GGCTTACCAAGGCGACGATCTC |
| | | | TAGCTGGTCTGAGAGGATGACC |
| | | | AGCCACACTGGAACTGAGACAC |
| | | | GGTCCAGACTCCTACGGGAGG |
| | | | CAGCAGTGGGGAATATTGCACA |
| | | | ATGGGGGAAACCCTGATGCAGC |
| | | | CATGCCGCGTGTATGAAGAAGG |
| | | | CCTTCGGGTTGTAAAGTACTTTC |
| | | | GGTGATGAGGAAGGTGGTGTAT |
| | | | CTAATAGGTGCATCAATTGACG |
| | | | TTAATTACAGAAGAAGCACCGG |
| | | | CTAACTCCGTGCCAGCAGCCGC |
| | | | GGTAATACGGAGGGTGCGAGC |
| | | | GTTAATCGGAATGACTGGGCGT |
| | | | AAAGGGCATGTAGGCGGATAAT |
| | | | TAAGTTAGGTGTGAAAGCCCTG |
| | | | GGCTCAACCTAGGAATTGCACT |
| | | | TAAAACTGGTTAACTAGAGTATT |
| | | | GTAGAGGAAGGTAGAATTCCAC |
| | | | GTGTAGCGGTGAAATGCGTAGA |
| | | | GATGTGGAGGAATACCGGTGG |
| | | | CGAAGGCGGCCTTCTGGACAG |
| | | | ATACTGACGCTGAGATGCGAAA |
| | | | GCGTGGGGAGCAAACAGGATTA |
| | | | GATACCCTGGTAGTCCACGCTG |
| | | | TAAACGATGTCGATTTGGAGTTT |
| | | | GTTGCCTAGAGTGATGGGCTCC |
| | | | GAAGCTAACGCGATAAATCGAC |
| | | | CGCCTGGGGAGTACGGCCGCA |
| | | | AGGTTAAAACTCAAATGAATTGA |
| | | | CGGGGGCCCGCACAAGCGGTG |
| | | | GAGCATGTGGTTTAATTCGATG |
| | | | CAACGCGAAGAACCTTACCTGG |
| | | | TCTTGACATCCACAGAATCTTGC |
| | | | AGAGATGCGGGAGTGCCTTCG |
| | | | GGAACTGTGAGACAGGTGCTGC |
| | | | ATGGCTGTCGTCAGCTCGTGTT |
| | | | GTGAAATGTTGGGTTAAGTCCC |
| | | | GCAACGAGCGCAACCCTTATCC |
| | | | TTTGTTGCCATCGGTTAGGCCG |
| | | | GGAACTCAAAGGAGACTGCCGT |
| | | | TGATAAAGCGGAGGAAGGTGG |
| | | | GGACGACGTCAAGTCATCATGG |
| | | | CCCTTACGACCAGGGCTACACA |
| | | | CGTGCTACAATGGCGTATACAA |
| | | | AGGGAGGCGACCTCGCGAGAG |
| | | | CAAGCGGACCTCATAAAGTACG |
| | | | TCTAAGTCCGGATTGGAGTCTG |
| | | | CAACTCGACTCCATGAAGTCGG |
| | | | AATCGCTAGTAATCGTGAATCA |
| | | | GAATGTCACGGTGAATACGTTC |
| | | | CCGGGCCTTGTACACACCGCCC |
| | | | GTCACACCATGGGAGTGGGTTG |
| | | | CACCAGAAGTAGATAGCTTAAC |
| | | | CTTCGGGAGGGCGTTTACCACG |
| | | | GTGTGGTCCATGACTGGGGTGA |
| | | | AGTCGTAACAAGGTAACCGTAG |
| | | | GGGAACCTGCGGTTGGATCACC |
| | | | TCCTTAC |
| | | | (SEQ ID NO: 34) |
| Bartonella apis | honeybee (Apis | Gut | AAGCCAAAATCAAATTTTCAACT |
| | mellifera) | | TGAGAGTTTGATCCTGGCTCAG |
| | | | AACGAACGCTGGCGGCAGGCT |
| | | | TAACACATGCAAGTCGAACGCA |
| | | | CTTTTCGGAGTGAGTGGCAGAC |
| | | | GGGTGAGTAACGCGTGGGAAT |
| | | | CTACCTATTTCTACGGAATAACG |
| | | | CAGAGAAATTTGTGCTAATACC |
| | | | GTATACGTCCTTCGGGAGAAAG |
| | | | ATTTATCGGAGATAGATGAGCC |
| | | | CGCGTTGGATTAGCTAGTTGGT |
| | | | GAGGTAATGGCCCACCAAGGC |
| | | | GACGATCCATAGCTGGTCTGAG |
| | | | AGGATGACCAGCCACATTGGGA |
| | | | CTGAGACACGGCCCAGACTCCT |
| | | | ACGGGAGGCAGCAGTGGGGAA |
| | | | TATTGGACAATGGGCGCAAGCC |
| | | | TGATCCAGCCATGCCGCGTGAG |
| | | | TGATGAAGGCCCTAGGGTTGTA |
| | | | AAGCTCTTTCACCGGTGAAGAT |
| | | | AATGACGGTAACCGGAGAAGAA |
| | | | GCCCCGGCTAACTTCGTGCCAG |
| | | | CAGCCGCGGTAATACGAAGGG |
| | | | GGCTAGCGTTGTTCGGATTTAC |
| | | | TGGGCGTAAAGCGCACGTAGG |
| | | | CGGATATTTAAGTCAGGGGTGA |
| | | | AATCCCGGGGCTCAACCCCGG |
| | | | AACTGCCTTTGATACTGGATATC |
| | | | TTGAGTATGGAAGAGGTAAGTG |
| | | | GAATTCCGAGTGTAGAGGTGAA |
| | | | ATTCGTAGATATTCGGAGGAAC |
| | | | ACCAGTGGCGAAGGCGGCTTA |
| | | | CTGGTCCATTACTGACGCTGAG |
| | | | GTGCGAAAGCGTGGGGAGCAA |
| | | | ACAGGATTAGATACCCTGGTAG |
| | | | TCCACGCTGTAAACGATGAATG |
| | | | TTAGCCGTTGGACAGTTTACTG |
| | | | TTCGGTGGCGCAGCTAACGCAT |
| | | | TAAACATTCCGCCTGGGGAGTA |
| | | | CGGTCGCAAGATTAAAACTCAA |
| | | | AGGAATTGACGGGGGCCCGCA |
| | | | CAAGCGGTGGAGCATGTGGTTT |
| | | | AATTCGAAGCAACGCGCAGAAC |
| | | | CTTACCAGCCCTTGACATCCCG |
| | | | ATCGCGGATGGTGGAGACACC |
| | | | GTCTTTCAGTTCGGCTGGATCG |
| | | | GTGACAGGTGCTGCATGGCTGT |
| | | | CGTCAGCTCGTGTCGTGAGATG |
| | | | TTGGGTTAAGTCCCGCAACGAG |
| | | | CGCAACCCTCGCCCTTAGTTGC |
| | | | CATCATTTAGTTGGGCACTCTAA |
| | | | GGGGACTGCCGGTGATAAGCC |
| | | | GAGAGGAAGGTGGGGATGACG |
| | | | TCAAGTCCTCATGGCCCTTACG |
| | | | GGCTGGGCTACACACGTGCTAC |
| | | | AATGGTGGTGACAGTGGGCAG |
| | | | CGAGACCGCGAGGTCGAGCTA |
| | | | ATCTCCAAAAGCCATCTCAGTTC |
| | | | GGATTGCACTCTGCAACTCGAG |
| | | | TGCATGAAGTTGGAATCGCTAG |
| | | | TAATCGTGGATCAGCATGCCAC |
| | | | GGTGAATACGTTCCCGGGCCTT |
| | | | GTACACACCGCCCGTCACACCA |
| | | | TGGGAGTTGGTTTTACCCGAAG |
| | | | GTGCTGTGCTAACCGCAAGGAG |
| | | | GCAGGCAACCACGGTAGGGTC |
| | | | AGCGACTGGGGTGAAGTCGTAA |
| | | | CAAGGTAGCCGTAGGGGAACCT |
| | | | GCGGCTGGATCACCTCCTTTCT |
| | | | AAGGAAGATGAAGAATTGGAA |
| | | | (SEQ ID NO: 35) |
| Parasaccharibacter | honeybee (Apis | Gut | CTACCATGCAAGTCGCACGAAA |
| apium | mellifera) | | CCTTTCGGGGTTAGTGGCGGAC |
| | | | GGGTGAGTAACGCGTTAGGAAC |
| | | | CTATCTGGAGGTGGGGGATAAC |
| | | | ATCGGGAAACTGGTGCTAATAC |
| | | | CGCATGATGCCTGAGGGCCAAA |
| | | | GGAGAGATCCGCCATTGGAGG |
| | | | GGCCTGCGTTCGATTAGCTAGT |
| | | | TGGTTGGGTAAAGGCTGACCAA |
| | | | GGCGATGATCGATAGCTGGTTT |
| | | | GAGAGGATGATCAGCCACACTG |
| | | | GGACTGAGACACGGCCCAGAC |
| | | | TCCTACGGGAGGCAGCAGTGG |
| | | | GGAATATTGGACAATGGGGGCA |
| | | | ACCCTGATCCAGCAATGCCGCG |
| | | | TGTGTGAAGAAGGTCTTCGGAT |
| | | | TGTAAAGCACTTTCACTAGGGA |
| | | | AGATGATGACGGTACCTAGAGA |
| | | | AGAAGCCCCGGCTAACTTCGTG |
| | | | CCAGCAGCCGCGGTAATACGAA |
| | | | GGGGGCTAGCGTTGCTCGGAA |
| | | | TGACTGGGCGTAAAGGGCGCG |
| | | | TAGGCTGTTTGTACAGTCAGAT |
| | | | GTGAAATCCCCGGGCTTAACCT |
| | | | GGGAACTGCATTTGATACGTGC |
| | | | AGACTAGAGTCCGAGAGAGGGT |
| | | | TGTGGAATTCCCAGTGTAGAGG |
| | | | TGAAATTCGTAGATATTGGGAA |
| | | | GAACACCGGTTGCGAAGGCGG |
| | | | CAACCTGGCTNNNNNNNNNNNN |
| | | | NNNNNNNNNNNNNNNNNNNNN |
| | | | NNNNNNNNNNNNNNNNNNNNN |
| | | | NNNNNNNNNNNNNNNNNNNNN |
| | | | NNNNNNNNNNNNNNNNNNNNN |
| | | | NNNNNNNNNNNNNNNNNNGAG |
| | | | CTAACGCGTTAAGCACACCGCC |
| | | | TGGGGAGTACGGCCGCAAGGT |
| | | | TGAAACTCAAAGGAATTGACGG |
| | | | GGGCCCGCACAAGCGGTGGAG |
| | | | CATGTGGTTTAATTCGAAGCAA |
| | | | CGCGCAGAACCTTACCAGGGCT |
| | | | TGCATGGGGAGGCTGTATTCAG |
| | | | AGATGGATATTTCTTCGGACCT |
| | | | CCCGCACAGGTGCTGCATGGCT |
| | | | GTCGTCAGCTCGTGTCGTGAGA |
| | | | TGTTGGGTTAAGTCCCGCAACG |
| | | | AGCGCAACCCTTGTCTTTAGTT |
| | | | GCCATCACGTCTGGGTGGGCA |
| | | | CTCTAGAGAGACTGCCGGTGAC |
| | | | AAGCCGGAGGAAGGTGGGGAT |
| | | | GACGTCAAGTCCTCATGGCCCT |
| | | | TATGTCCTGGGCTACACACGTG |
| | | | CTACAATGGCGGTGACAGAGG |
| | | | GATGCTACATGGTGACATGGTG |
| | | | CTGATCTCAAAAAACCGTCTCA |
| | | | GTTCGGATTGTACTCTGCAACT |
| | | | CGAGTGCATGAAGGTGGAATCG |
| | | | CTAGTAATCGCGGATCAGCATG |
| | | | CCGCGGTGAATACGTTCCCGG |
| | | | GCCTTGTACACACCGCCCGTCA |
| | | | CACCATGGGAGTTGGTTTGACC |
| | | | TTAAGCCGGTGAGCGAACCGCA |
| | | | AGGAACGCAGCCGACCACCGG |
| | | | TTCGGGTTCAGCGACTGGGGGA |
| | | | (SEQ ID NO: 36) |
| Lactobacillus kunkeei | honeybee (Apis | Gut | TTCCTTAGAAAGGAGGTGATCC |
| | mellifera) | | AGCCGCAGGTTCTCCTACGGCT |
| | | | ACCTTGTTACGACTTCACCCTAA |
| | | | TCATCTGTCCCACCTTAGACGA |
| | | | CTAGCTCCTAAAAGGTTACCCC |
| | | | ATCGTCTTTGGGTGTTACAAACT |
| | | | CTCATGGTGTGACGGGCGGTGT |
| | | | GTACAAGGCCCGGGAACGTATT |
| | | | CACCGTGGCATGCTGATCCACG |
| | | | ATTACTAGTGATTCCAACTTCAT |
| | | | GCAGGCGAGTTGCAGCCTGCA |
| | | | ATCCGAACTGAGAATGGCTTTA |
| | | | AGAGATTAGCTTGACCTCGCGG |
| | | | TTTCGCGACTCGTTGTACCATC |
| | | | CATTGTAGCACGTGTGTAGCCC |
| | | | AGCTCATAAGGGGCATGATGAT |
| | | | TTGACGTCGTCCCCACCTTCCT |
| | | | CCGGTTTATCACCGGCAGTCTC |
| | | | ACTAGAGTGCCCAACTAAATGC |
| | | | TGGCAACTAATAATAAGGGTTG |
| | | | CGCTCGTTGCGGGACTTAACCC |
| | | | AACATCTCACGACACGAGCTGA |
| | | | CGACAACCATGCACCACCTGTC |
| | | | ATTCTGTCCCCGAAGGGAACGC |
| | | | CCAATCTCTTGGGTTGGCAGAA |
| | | | GATGTCAAGAGCTGGTAAGGTT |
| | | | CTTCGCGTAGCATCGAATTAAA |
| | | | CCACATGCTCCACCACTTGTGC |
| | | | GGGCCCCCGTCAATTCCTTTGA |
| | | | GTTTCAACCTTGCGGTCGTACT |
| | | | CCCCAGGCGGAATACTTAATGC |
| | | | GTTAGCTGCGGCACTGAAGGG |
| | | | CGGAAACCCTCCAACACCTAGT |
| | | | ATTCATCGTTTACGGCATGGAC |
| | | | TACCAGGGTATCTAATCCTGTTC |
| | | | GCTACCCATGCTTTCGAGCCTC |
| | | | AGCGTCAGTAACAGACCAGAAA |
| | | | GCCGCCTTCGCCACTGGTGTTC |
| | | | TTCCATATATCTACGCATTTCAC |
| | | | CGCTACACATGGAGTTCCACTT |
| | | | TCCTCTTCTGTACTCAAGTTTTG |
| | | | TAGTTTCCACTGCACTTCCTCAG |
| | | | TTGAGCTGAGGGCTTTCACAGC |
| | | | AGACTTACAAAACCGCCTGCGC |
| | | | TCGCTTTACGCCCAATAAATCC |
| | | | GGACAACGCTTGCCACCTACGT |
| | | | ATTACCGCGGCTGCTGGCACGT |
| | | | AGTTAGCCGTGGCTTTCTGGTT |
| | | | AAATACCGTCAAAGTGTTAACA |
| | | | GTTACTCTAACACTTGTTCTTCT |
| | | | TTAACAACAGAGTTTTACGATCC |
| | | | GAAAACCTTCATCACTCACGCG |
| | | | GCGTTGCTCCATCAGACTTTCG |
| | | | TCCATTGTGGAAGATTCCCTACT |
| | | | GCTGCCTCCCGTAGGAGTCTGG |
| | | | GCCGTGTCTCAGTCCCAATGTG |
| | | | GCCGATTACCCTCTCAGGTCGG |
| | | | CTACGTATCATCGTCTTGGTGG |
| | | | GCTTTTATCTCACCAACTAACTA |
| | | | ATACGGCGCGGGTCCATCCCAA |
| | | | AGTGATAGCAAAGCCATCTTTC |
| | | | AAGTTGGAACCATGCGGTTCCA |
| | | | ACTAATTATGCGGTATTAGCACT |
| | | | TGTTTCCAAATGTTATCCCCCGC |
| | | | TTCGGGGCAGGTTACCCACGTG |
| | | | TTACTCACCAGTTCGCCACTCG |
| | | | CTCCGAATCCAAAAATCATTTAT |
| | | | GCAAGCATAAAATCAATTTGGG |
| | | | AGAACTCGTTCGACTTGCATGT |
| | | | ATTAGGCACGCCGCCAGCGTTC |
| | | | GTCCTGAGCCAGGATCAAACTC |
| | | | TCATCTTAA |
| | | | (SEQ ID NO: 37) |
| Lactobacillus Firm-4 | honeybee (Apis | Gut | ACGAACGCTGGCGGCGTGCCT |
| | mellifera) | | AATACATGCAAGTCGAGCGCGG |
| | | | GAAGTCAGGGAAGCCTTCGGGT |
| | | | GGAACTGGTGGAACGAGCGGC |
| | | | GGATGGGTGAGTAACACGTAGG |
| | | | TAACCTGCCCTAAAGCGGGGGA |
| | | | TACCATCTGGAAACAGGTGCTA |
| | | | ATACCGCATAAACCCAGCAGTC |
| | | | ACATGAGTGCTGGTTGAAAGAC |
| | | | GGCTTCGGCTGTCACTTTAGGA |
| | | | TGGACCTGCGGCGTATTAGCTA |
| | | | GTTGGTGGAGTAACGGTTCACC |
| | | | AAGGCAATGATACGTAGCCGAC |
| | | | CTGAGAGGGTAATCGGCCACAT |
| | | | TGGGACTGAGACACGGCCCAAA |
| | | | CTCCTACGGGAGGCAGCAGTA |
| | | | GGGAATCTTCCACAATGGACGC |
| | | | AAGTCTGATGGAGCAACGCCGC |
| | | | GTGGATGAAGAAGGTCTTCGGA |
| | | | TCGTAAAATCCTGTTGTTGAAGA |
| | | | AGAACGGTTGTGAGAGTAACTG |
| | | | CTCATAACGTGACGGTAATCAA |
| | | | CCAGAAAGTCACGGCTAACTAC |
| | | | GTGCCAGCAGCCGCGGTAATAC |
| | | | GTAGGTGGCAAGCGTTGTCCG |
| | | | GATTTATTGGGCGTAAAGGGAG |
| | | | CGCAGGCGGTCTTTTAAGTCTG |
| | | | AATGTGAAAGCCCTCAGCTTAA |
| | | | CTGAGGAAGAGCATCGGAAACT |
| | | | GAGAGACTTGAGTGCAGAAGAG |
| | | | GAGAGTGGAACTCCATGTGTAG |
| | | | CGGTGAAATGCGTAGATATATG |
| | | | GAAGAACACCAGTGGCGAAGG |
| | | | CGGCTCTCTGGTCTGTTACTGA |
| | | | CGCTGAGGCTCGAAAGCATGG |
| | | | GTAGCGAACAGGATTAGATACC |
| | | | CTGGTAGTCCATGCCGTAAACG |
| | | | ATGAGTGCTAAGTGTTGGGAGG |
| | | | TTTCCGCCTCTCAGTGCTGCAG |
| | | | CTAACGCATTAAGCACTCCGCC |
| | | | TGGGGAGTACGACCGCAAGGTT |
| | | | GAAACTCAAAGGAATTGACGGG |
| | | | GGCCCGCACAAGCGGTGGAGC |
| | | | ATGTGGTTTAATTCGAAGCAAC |
| | | | GCGAAGAACCTTACCAGGTCTT |
| | | | GACATCTCCTGCAAGCCTAAGA |
| | | | GATTAGGGGTTCCCTTCGGGGA |
| | | | CAGGAAGACAGGTGGTGCATG |
| | | | GTTGTCGTCAGCTCGTGTCGTG |
| | | | AGATGTTGGGTTAAGTCCCGCA |
| | | | ACGAGCGCAACCCTTGTTACTA |
| | | | GTTGCCAGCATTAAGTTGGGCA |
| | | | CTCTAGTGAGACTGCCGGTGAC |
| | | | AAACCGGAGGAAGGTGGGGAC |
| | | | GACGTCAAATCATCATGCCCCT |
| | | | TATGACCTGGGCTACACACGTG |
| | | | CTACAATGGATGGTACAATGAG |
| | | | AAGCGAACTCGCGAGGGGAAG |
| | | | CTGATCTCTGAAAACCATTCTCA |
| | | | GTTCGGATTGCAGGCTGCAACT |
| | | | CGCCTGCATGAAGCTGGAATCG |
| | | | CTAGTAATCGCGGATCAGCATG |
| | | | CCGCGGTGAATACGTTCCCGG |
| | | | GCCTTGTACACACCGCCC |
| | | | (SEQ ID NO: 38) |
| Silk worm | | | |
| Enterococcus | Bombyx mori | Gut | AGGTGATCCAGCCGCACCTTCC |
| | | | GATACGGCTACCTTGTTACGAC |
| | | | TTCACCCCAATCATCTATCCCAC |
| | | | CTTAGGCGGCTGGCTCCAAAAA |
| | | | GGTTACCTCACCGACTTCGGGT |
| | | | GTTACAAACTCTCGTGGTGTGA |
| | | | CGGGCGGTGTGTACAAGGCCC |
| | | | GGGAACGTATTCACCGCGGCGT |
| | | | GCTGATCCGCGATTACTAGCGA |
| | | | TTCCGGCTTCATGCAGGCGAGT |
| | | | TGCAGCCTGCAATCCGAACTGA |
| | | | GAGAAGCTTTAAGAGATTTGCA |
| | | | TGACCTCGCGGTCTAGCGACTC |
| | | | GTTGTACTTCCCATTGTAGCAC |
| | | | GTGTGTAGCCCAGGTCATAAGG |
| | | | GGCATGATGATTTGACGTCATC |
| | | | CCCACCTTCCTCCGGTTTGTCA |
| | | | CCGGCAGTCTCGCTAGAGTGCC |
| | | | CAACTAAATGATGGCAACTAAC |
| | | | AATAAGGGTTGCGCTCGTTGCG |
| | | | GGACTTAACCCAACATCTCACG |
| | | | ACACGAGCTGACGACAACCATG |
| | | | CACCACCTGTCACTTTGTCCCC |
| | | | GAAGGGAAAGCTCTATCTCTAG |
| | | | AGTGGTCAAAGGATGTCAAGAC |
| | | | CTGGTAAGGTTCTTCGCGTTGC |
| | | | TTCGAATTAAACCACATGCTCCA |
| | | | CCGCTTGTGCGGGCCCCCGTC |
| | | | AATTCCTTTGAGTTTCAACCTTG |
| | | | CGGTCGTACTCCCCAGGCGGA |
| | | | GTGCTTAATGCGTTTGCTGCAG |
| | | | CACTGAAGGGCGGAAACCCTCC |
| | | | AACACTTAGCACTCATCGTTTAC |
| | | | GGCGTGGACTACCAGGGTATCT |
| | | | AATCCTGTTTGCTCCCCACGCT |
| | | | TTCGAGCCTCAGCGTCAGTTAC |
| | | | AGACCAGAGAGCCGCCTTCGC |
| | | | CACTGGTGTTCCTCCATATATCT |
| | | | ACGCATTTCACCGCTACACATG |
| | | | GAATTCCACTCTCCTCTTCTGCA |
| | | | CTCAAGTCTCCCAGTTTCCAAT |
| | | | GACCCTCCCCGGTTGAGCCGG |
| | | | GGGCTTTCACATCAGACTTAAG |
| | | | AAACCGCCTGCGCTCGCTTTAC |
| | | | GCCCAATAAATCCGGACAACGC |
| | | | TTGCCACCTACGTATTACCGCG |
| | | | GCTGCTGGCACGTAGTTAGCCG |
| | | | TGGCTTTCTGGTTAGATACCGT |
| | | | CAGGGGACGTTCAGTTACTAAC |
| | | | GTCCTTGTTCTTCTCTAACAACA |
| | | | GAGTTTTACGATCCGAAAACCTT |
| | | | CTTCACTCACGCGGCGTTGCTC |
| | | | GGTCAGACTTTCGTCCATTGCC |
| | | | GAAGATTCCCTACTGCTGCCTC |
| | | | CCGTAGGAGTCTGGGCCGTGT |
| | | | CTCAGTCCCAGTGTGGCCGATC |
| | | | ACCCTCTCAGGTCGGCTATGCA |
| | | | TCGTGGCCTTGGTGAGCCGTTA |
| | | | CCTCACCAACTAGCTAATGCAC |
| | | | CGCGGGTCCATCCATCAGCGAC |
| | | | ACCCGAAAGCGCCTTTCACTCT |
| | | | TATGCCATGCGGCATAAACTGT |
| | | | TATGCGGTATTAGCACCTGTTTC |
| | | | CAAGTGTTATCCCCCTCTGATG |
| | | | GGTAGGTTACCCACGTGTTACT |
| | | | CACCCGTCCGCCACTCCTCTTT |
| | | | CCAATTGAGTGCAAGCACTCGG |
| | | | GAGGAAAGAAGCGTTCGACTTG |
| | | | CATGTATTAGGCACGCCGCCAG |
| | | | CGTTCGTCCTGAGCCAGGATCA |
| | | | AACTCT |
| | | | (SEQ ID NO: 39) |
| Delftia | Bombyx mori | Gut | CAGAAAGGAGGTGATCCAGCC |
| | | | GCACCTTCCGATACGGCTACCT |
| | | | TGTTACGACTTCACCCCAGTCA |
| | | | CGAACCCCGCCGTGGTAAGCG |
| | | | CCCTCCTTGCGGTTAGGCTACC |
| | | | TACTTCTGGCGAGACCCGCTCC |
| | | | CATGGTGTGACGGGCGGTGTG |
| | | | TACAAGACCCGGGAACGTATTC |
| | | | ACCGCGGCATGCTGATCCGCG |
| | | | ATTACTAGCGATTCCGACTTCAC |
| | | | GCAGTCGAGTTGCAGACTGCGA |
| | | | TCCGGACTACGACTGGTTTTAT |
| | | | GGGATTAGCTCCCCCTCGCGG |
| | | | GTTGGCAACCCTCTGTACCAGC |
| | | | CATTGTATGACGTGTGTAGCCC |
| | | | CACCTATAAGGGCCATGAGGAC |
| | | | TTGACGTCATCCCCACCTTCCT |
| | | | CCGGTTTGTCACCGGCAGTCTC |
| | | | ATTAGAGTGCTCAACTGAATGTA |
| | | | GCAACTAATGACAAGGGTTGCG |
| | | | CTCGTTGCGGGACTTAACCCAA |
| | | | CATCTCACGACACGAGCTGACG |
| | | | ACAGCCATGCAGCACCTGTGTG |
| | | | CAGGTTCTCTTTCGAGCACGAA |
| | | | TCCATCTCTGGAAACTTCCTGC |
| | | | CATGTCAAAGGTGGGTAAGGTT |
| | | | TTTCGCGTTGCATCGAATTAAAC |
| | | | CACATCATCCACCGCTTGTGCG |
| | | | GGTCCCCGTCAATTCCTTTGAG |
| | | | TTTCAACCTTGCGGCCGTACTC |
| | | | CCCAGGCGGTCAACTTCACGCG |
| | | | TTAGCTTCGTTACTGAGAAAACT |
| | | | AATTCCCAACAACCAGTTGACAT |
| | | | CGTTTAGGGCGTGGACTACCAG |
| | | | GGTATCTAATCCTGTTTGCTCCC |
| | | | CACGCTTTCGTGCATGAGCGTC |
| | | | AGTACAGGTCCAGGGGATTGCC |
| | | | TTCGCCATCGGTGTTCCTCCGC |
| | | | ATATCTACGCATTTCACTGCTAC |
| | | | ACGCGGAATTCCATCCCCCTCT |
| | | | ACCGTACTCTAGCCATGCAGTC |
| | | | ACAAATGCAGTTCCCAGGTTGA |
| | | | GCCCGGGGATTTCACATCTGTC |
| | | | TTACATAACCGCCTGCGCACGC |
| | | | TTTACGCCCAGTAATTCCGATTA |
| | | | ACGCTCGCACCCTACGTATTAC |
| | | | CGCGGCTGCTGGCACGTAGTTA |
| | | | GCCGGTGCTTATTCTTACGGTA |
| | | | CCGTCATGGGCCCCCTGTATTA |
| | | | GAAGGAGCTTTTTCGTTCCGTA |
| | | | CAAAAGCAGTTTACAACCCGAA |
| | | | GGCCTTCATCCTGCACGCGGCA |
| | | | TTGCTGGATCAGGCTTTCGCCC |
| | | | ATTGTCCAAAATTCCCCACTGCT |
| | | | GCCTCCCGTAGGAGTCTGGGC |
| | | | CGTGTCTCAGTCCCAGTGTGGC |
| | | | TGGTCGTCCTCTCAGACCAGCT |
| | | | ACAGATCGTCGGCTTGGTAAGC |
| | | | TTTTATCCCACCAACTACCTAAT |
| | | | CTGCCATCGGCCGCTCCAATCG |
| | | | CGCGAGGCCCGAAGGGCCCCC |
| | | | GCTTTCATCCTCAGATCGTATG |
| | | | CGGTATTAGCTACTCTTTCGAGT |
| | | | AGTTATCCCCCACGACTGGGCA |
| | | | CGTTCCGATGTATTACTCACCC |
| | | | GTTCGCCACTCGTCAGCGTCCG |
| | | | AAGACCTGTTACCGTTCGACTT |
| | | | GCATGTGTAAGGCATGCCGCCA |
| | | | GCGTTCAATCTGAGCCAGGATC |
| | | | AAACTCTACAGTTCGATCT |
| | | | (SEQ ID NO: 40) |
| Pelomonas | Bombyx mori | Gut | ATCCTGGCTCAGATTGAACGCT |
| | | | GGCGGCATGCCTTACACATGCA |
| | | | AGTCGAACGGTAACAGGTTAAG |
| | | | CTGACGAGTGGCGAACGGGTG |
| | | | AGTAATATATCGGAACGTGCCC |
| | | | AGTCGTGGGGGATAACTGCTCG |
| | | | AAAGAGCAGCTAATACCGCATA |
| | | | CGACCTGAGGGTGAAAGCGGG |
| | | | GGATCGCAAGACCTCGCNNGAT |
| | | | TGGAGCGGCCGATATCAGATTA |
| | | | GGTAGTTGGTGGGGTAAAGGC |
| | | | CCACCAAGCCAACGATCTGTAG |
| | | | CTGGTCTGAGAGGACGACCAG |
| | | | CCACACTGGGACTGAGACACG |
| | | | GCCCAGACTCCTACGGGAGGC |
| | | | AGCAGTGGGGAATTTTGGACAA |
| | | | TGGGCGCAAGCCTGATCCAGC |
| | | | CATGCCGCGTGCGGGAAGAAG |
| | | | GCCTTCGGGTTGTAAACCGCTT |
| | | | TTGTCAGGGAAGAAAAGGTTCT |
| | | | GGTTAATACCTGGGACTCATGA |
| | | | CGGTACCTGAAGAATAAGCACC |
| | | | GGCTAACTACGTGCCAGCAGCC |
| | | | GCGGTAATACGTAGGGTGCAAG |
| | | | CGTTAATCGGAATTACTGGGCG |
| | | | TAAAGCGTGCGCAGGCGGTTAT |
| | | | GCAAGACAGAGGTGAAATCCCC |
| | | | GGGCTCAACCTGGGAACTGCCT |
| | | | TTGTGACTGCATAGCTAGAGTA |
| | | | CGGTAGAGGGGGATGGAATTC |
| | | | CGCGTGTAGCAGTGAAATGCGT |
| | | | AGATATGCGGAGGAACACCGAT |
| | | | GGCGAAGGCAATCCCCTGGAC |
| | | | CTGTACTGACGCTCATGCACGA |
| | | | AAGCGTGGGGAGCAAACAGGA |
| | | | TTAGATACCCTGGTAGTCCACG |
| | | | CCCTAAACGATGTCAACTGGTT |
| | | | GTTGGGAGGGTTTCTTCTCAGT |
| | | | AACGTANNTAACGCGTGAAGTT |
| | | | GACCGCCTGGGGAGTACGGCC |
| | | | GCAAGGTTGAAACTCAAAGGAA |
| | | | TTGACGGGGACCCGCACAAGC |
| | | | GGTGGATGATGTGGTTTAATTC |
| | | | GATGCAACGCGAAAAACCTTAC |
| | | | CTACCCTTGACATGCCAGGAAT |
| | | | CCTGAAGAGATTTGGGAGTGCT |
| | | | CGAAAGAGAACCTGGACACAGG |
| | | | TGCTGCATGGCCGTCGTCAGCT |
| | | | CGTGTCGTGAGATGTTGGGTTA |
| | | | AGTCCCGCAACGAGCGCAACC |
| | | | CTTGTCATTAGTTGCTACGAAAG |
| | | | GGCACTCTAATGAGACTGCCGG |
| | | | TGACAAACCGGAGGAAGGTGG |
| | | | GGATGACGTCAGGTCATCATGG |
| | | | CCCTTATGGGTAGGGCTACACA |
| | | | CGTCATACAATGGCCGGGACAG |
| | | | AGGGCTGCCAACCCGCGAGGG |
| | | | GGAGCTAATCCCAGAAACCCGG |
| | | | TCGTAGTCCGGATCGTAGTCTG |
| | | | CAACTCGACTGCGTGAAGTCGG |
| | | | AATCGCTAGTAATCGCGGATCA |
| | | | GCTTGCCGCGGTGAATACGTTC |
| | | | CCGGGTCTTGTACACACCGCCC |
| | | | GTCACACCATGGGAGCGGGTTC |
| | | | TGCCAGAAGTAGTTAGCCTAAC |
| | | | CGCAAGGAGGGCGATTACCAC |
| | | | GGCAGGGTTCGTGACTGGGGT |
| | | | GAAGTCGTAACAAGGTAGCCGT |
| | | | ATCGGAAGGTGCGGCTGGATCAC |
| | | | (SEQ ID NO: 41) |

Any number of bacterial species may be targeted by the compositions or methods described herein. For example, in some instances, the modulating agent may target a single bacterial species. In some instances, the modulating agent may target at least about any of 1, 2, 3, 4, 5, 6, 7, 8, 9, 10, 15, 20, 30, 40, 50, 60, 70, 80, 90, 100, 150, 200, 250, 500, or more distinct bacterial species. In some instances, the modulating agent may target any one of about 1 to about 5, about 5 to about 10, about 10 to about 20, about 20 to about 50, about 50 to about 100, about 100 to about 200, about 200 to about 500, about 10 to about 50, about 5 to about 20, or about 10 to about 100 distinct bacterial species. In some instances, the modulating agent may target at least about any of 1, 2, 3, 4, 5, 6, 7, 8, 9, 10, 15, 20, 30, 40, 50, 60, 70, 80, 90, 100, or more phyla, classes, orders, families, or genera of bacteria.

In some instances, the modulating agent may increase a population of one or more bacteria (e.g., pathogenic bacteria, toxin-producing bacteria) by at least about any of 10%, 20%, 30%, 40%, 50%, 60%, 70%, 80%, 90% or more in the host in comparison to a host organism to which the modulating agent has not been administered. In some instances, the modulating agent may reduce the population of one or more bacteria (e.g., symbiotic bacteria, a pesticide-degrading bacterium (e.g., a bacterium that degrades a pesticide listed in Table 12) by at least about any of 10%, 20%, 30%, 40%, 50%, 60%, 70%, 80%, 90%, or more in the host in comparison to a host organism to which the modulating agent has not been administered. In some instances, the modulating agent may eradicate the population of a bacterium (e.g., a symbiotic bacterium, a pesticide-degrading bacterium) in the host. In some instances, the modulating agent may increase the level of one or more pathogenic bacteria by at least about any of 10%, 20%, 30%, 40%, 50%, 60%, 70%, 80%, 90% or more in the host and/or decreases the level of one or more symbiotic bacteria by at least about any of 10%, 20%, 30%, 40%, 50%, 60%, 70%, 80%, 90%, or more in the host in comparison to a host organism to which the modulating agent has not been administered.

In some instances, the modulating agent may alter the bacterial diversity and/or bacterial composition of the host. In some instances, the modulating agent may increase the bacterial diversity in the host relative to a starting diversity by at least about any of 10%, 20%, 30%, 40%, 50%, 60%, 70%, 80%, 90%, or more in comparison to a host organism to which the modulating agent has not been administered. In some instances, the modulating agent may decrease the bacterial diversity in the host relative to a starting diversity by at least about any of 10%, 20%, 30%, 40%, 50%, 60%, 70%, 80%, 90%, or more in comparison to a host organism to which the modulating agent has not been administered.

In some instances, the modulating agent may alter the function, activity, growth, and/or division of one or more bacterial cells. For example, the modulating agent may alter the expression of one or genes in the bacteria. In some instances, the modulating agent may alter the function of one or more proteins in the bacteria. In some instances, the modulating agent may alter the function of one or more cellular structures (e.g., the cell wall, the outer or inner membrane) in the bacteria. In some instances, the modulating agent may kill (e.g., lyse) the bacteria.

The target bacterium may reside in one or more parts of the insect. Further, the target bacteria may be intracellular or extracellular. In some instances, the bacteria reside in or on one or more parts of the host gut, including, e.g., the foregut, midgut, and/or hindgut. In some instances, the bacteria reside as an intracellular bacteria within a cell of the host insect. In some instances, the bacteria reside in a bacteriocyte of the host insect.

Changes to the populations of bacteria in the host may be determined by any methods known in the art, such as microarray, polymerase chain reaction (PCR), real-time PCR, flow cytometry, fluorescence microscopy, transmission electron microscopy, fluorescence in situ hybridization (e.g., FISH), spectrophotometry, matrix-assisted laser desorption ionization-mass spectrometry (MALDI-MS), and DNA sequencing. In some instances, a sample of the host treated with a modulating agent is sequenced (e.g., by metagenomics sequencing of 16S rRNA or rDNA) to determine the microbiome of the host after delivery or administration of the modulating agent. In some instances, a sample of a host that did not receive the modulating agent is also sequenced to provide a reference.

ii. Fungi

Exemplary fungi that may be targeted in accordance with the methods and compositions provided herein, include, but are not limited to Amylostereum areolatum, Epichloe spp, Pichia pinus, Hansenula capsulate, Daldinia decipien, Ceratocytis spp, Ophiostoma spp, and Attamyces bromatificus. Non-limiting examples of yeast and yeast-like symbionts found in insects include Candida, Metschnikowia, Debaromyces, Scheffersomyces shehatae and Scheffersomyces stipites, Starmerella, Pichia, Trichosporon, Cryptococcus, Pseudozyma, and yeast-like symbionts from the subphylum Pezizomycotina (e.g., Symbiotaphrina bucneri and Symbiotaphrina kochil). Non-limiting examples of yeast that may be targeted by the methods and compositions herein are listed in Table 2.

TABLE 2
Examples of Yeast in Insects

| Insect Species | Order: Family | Yeast Location (Species) |
|---|---|---|
| Stegobium paniceum | Coleoptera: Anobiidae | Mycetomes |
| (=Sitodrepa panicea) | | (Saccharomyces) |
| | | Cecae (Torulopsis buchnerii) |
| | | Mycetome between foregut and midgut |
| | | Mycetomes (Symbiotaphrina buchnerii) |
| | | Mycetomes and digestive tube |
| | | (Torulopsis buchnerii) |
| | | Gut cecae (Symbiotaphrina buchnerii) |
| Lasioderma serricorne | Coleoptera: Anobiidae | Mycetome between foregut and midgut |
| | | (Symbiotaphrina kochii) |
| Ernobius abietis | Coleoptera: Anobiidae | Mycetomes (Torulopsis karawaiewii) |
| | | (Candida karawaiewii) |
| Ernobius mollis | Coleoptera: Anobiidae | Mycetomes (Torulopsis ernobii) |
| | | (Candida ernobii) |
| Hemicoelus gibbicollis | Coleoptera: Anobiidae | Larval mycetomes |
| Xestobium plumbeum | Coleoptera: Anobiidae | Mycetomes (Torulopsis xestobii) |
| | | (Candida xestobii) |
| Criocephalus rusticus | Coleoptera: Cerambycidae | Mycetomes |
| Phoracantha | Coleoptera: Cerambycidae | Alimentary canal (Candida |
| semipunctata | | guilliermondii, C. tenuis) |
| | | Cecae around midgut (Candida |
| | | guilliermondii) |
| Harpium inquisitor | Coleoptera: Cerambycidae | Mycetomes (Candida rhagii) |
| Harpium mordax | Coleoptera: Cerambycidae | Cecae around midgut (Candida tenuis) |
| H. sycophanta | | |
| Gaurotes virginea | Coleoptera: Cerambycidae | Cecae around midgut (Candida rhagii) |
| Leptura rubra | Coleoptera: Cerambycidae | Cecae around midgut (Candida tenuis) |
| | | Cecae around midgut (Candida |
| | | parapsilosis) |
| Leptura maculicornis | Coleoptera: Cerambycidae | Cecae around midgut (Candida |
| | | parapsilosis) |
| L. cerambyciformis | | |
| Leptura sanguinolenta | Coleoptera: Cerambycidae | Cecae around midgut (Candida sp.) |
| Rhagium bifasciatum | Coleoptera: Cerambycidae | Cecae around midgut (Candida tenuis) |
| Rhagium inquisitor | Coleoptera: Cerambycidae | Cecae around midgut (Candida |
| | | guilliermondii) |
| Rhagium mordax | Coleoptera: Cerambycidae | Cecae around midgut (Candida) |
| Carpophilus | Coleoptera: Nitidulidae | Intestinal tract (10 yeast species) |
| hemipterus | | |
| Odontotaenius | Coleoptera: Passalidae | Hindgut (Enteroramus dimorphus) |
| disjunctus | | |
| Odontotaenius | Coleoptera: Passalidae | Gut (Pichia stipitis, P. segobiensis, |
| disjunctus | | Candida shehatae) |
| Verres stembergianus | | (C. ergatensis) |
| Scarabaeus | Coleoptera: Scarabaeidae | Digestive tract (10 yeast species) |
| semipunctatus | | |
| Chironitis furcifer | | |
| Unknown species | Coleoptera: Scarabaeidae | Guts (Trichosporon cutaneum) |
| Dendroctonus and Ips | Coleoptera: Scolytidae | Alimentary canal (13 yeast species) |
| spp. | | |
| Dendroctonus frontalis | Coleoptera: Scolytidae | Midgut (Candida sp.) |
| Ips sexdentatus | Coleoptera: Scolytidae | Digestive tract (Pichia bovis, P. |
| | | rhodanensis) |
| | | Hansenula holstii (Candida rhagii) |
| | | Digestive tract |
| | | (Candida pulcherina) |
| Ips typographus | Coleoptera: Scolytidae | Alimentary canal |
| | | Alimentary tracts (Hansenula capsulata, |
| | | Candida parapsilosis) |
| | | Guts and beetle homogenates |
| | | (Hansenula holstii, H. capsulata, |
| | | Candida diddensii, C. mohschtana, C. |
| | | nitratophila, Cryptococcus albidus, C. |
| | | laurentii) |
| Trypodendron | Coleoptera: Scolytidae | Not specified |
| lineatum | | |
| Xyloterinus politus | Coleoptera: Scolytidae | Head, thorax, abdomen (Candida, |
| | | Pichia, Saccharomycopsis) |
| Periplaneta americana | Dictyoptera: Blattidae | Hemocoel (Candida sp. nov.) |
| Blatta orientalis | Dictyoptera: Blattidae | Intestinal tract (Kluyveromyces blattae) |
| Blatella germanica | Dictyoptera: Blattellidae | Hemocoel |
| Cryptocercus | Dictyoptera: Cryptocercidae | Hindgut (1 yeast species) |
| punctulatus | | |
| Philophylla heraclei | Diptera: Tephritidae | Hemocoel |
| Aedes (4 species) | Diptera: Culicidae | Internal microflora (9 yeast genera) |
| Drosophila | Diptera: Drosophilidae | Alimentary canal (24 yeast species) |
| pseudoobscura | | |
| Drosophila (5 spp.) | Diptera: Drosophilidae | Crop (42 yeast species) |
| Drosophila | Diptera: Drosophilidae | Crop (8 yeast species) |
| melanogaster | | |
| Drosophila (4 spp.) | Diptera: Drosophilidae | Crop (7 yeast species) |
| Drosophila (6 spp.) | Diptera: Drosophilidae | Larval gut (17 yeast species) |
| Drosophila (20 spp.) | Diptera: Drosophilidae | Crop (20 yeast species) |
| Drosophila (8 species | Diptera: Drosophilidae | Crop (Kloeckera, Candida, |
| groups) | | Kluyveromyces) |
| Drosophila serido | Diptera: Drosophilidae | Crop (18 yeast species) |
| Drosophila (6 spp.) | Diptera: Drosophilidae | Intestinal epithelium (Coccidiascus |
| | | legeri) |
| Protaxymia | Diptera | Unknown (Candida, Cryptococcus, |
| melanoptera | | Sporoblomyces) |
| Astegopteryx styraci | Homoptera: Aphididae | Hemocoel and fat body |
| Tuberaphis sp. | Homoptera: Aphididae | Tissue sections |
| Hamiltonaphis styraci | | |
| Glyphinaphis | | |
| bambusae | | |
| Cerataphis sp. | | |
| Hamiltonaphis styraci | Homoptera: Aphididae | Abdominal hemocoel |
| Cofana unimaculata | Homoptera: Cicadellidae | Fat body |
| Leofa unicolor | Homoptera: Cicadellidae | Fat body |
| Lecaniines, etc. | Homoptera: Coccoidea d | Hemolymph, fatty tissue, etc. |
| Lecanium sp. | Homoptera: Coccidae | Hemolymph, adipose tissue |
| Ceroplastes (4 sp.) | Homoptera: Coccidae | Blood smears |
| Laodelphax striatellus | Homoptera: Delphacidae | Fat body |
| | | Eggs |
| | | Eggs (Candida) |
| Nilaparvata lugens | Homoptera: Delphacidae | Fat body |
| | | Eggs (2 unidentified yeast species) |
| | | Eggs, nymphs (Candida) |
| | | Eggs (7 unidentified yeast species) |
| | | Eggs (Candida) |
| Nisia nervosa | Homoptera: Delphacidae | Fat body |
| Nisia grandiceps | | |
| Perkinsiella spp. | | |
| Sardia rostrata | | |
| Sogatella furcifera | | |
| Sogatodes orizicola | Homoptera: Delphacidae | Fat body |
| Amrasca devastans | Homoptera: Jassidae | Eggs, mycetomes, hemolymph |
| Tachardina lobata | Homoptera: Kerriidae | Blood smears (Torulopsis) |
| Laccifer (=Lakshadia) | Homoptera: Kerriidae | Blood smears (Torula variabilis) |
| sp. | | |
| Comperia merceti | Hymenoptera: Encyrtidae | Hemolymph, gut, poison gland |
| Solenopsis invicta | Hymenoptera: Formicidae | Hemolymph (Myrmecomyces |
| | | annellisae) |
| S. quinquecuspis | | |
| Solenopsis invicta | Hymenoptera: Formicidae | Fourth instar larvae (Candida |
| | | parapsilosis, Yarrowia lipolytica) |
| | | Gut and hemolymph (Candida |
| | | parapsilosis, C. lipolytica, C. |
| | | guillermondii, C. rugosa, Debaryomyces |
| | | hansenii) |
| Apis mellifera | Hymenoptera: Apidae | Digestive tracts (Torulopsis sp.) |
| | | Intestinal tract (Torulopsis apicola) |
| | | Digestive tracts (8 yeast species) |
| | | Intestinal contents (12 yeast species) |
| | | Intestinal contents (7 yeast species) |
| | | Intestines (14 yeast species) |
| | | Intestinal tract (Pichia melissophila) |
| | | Intestinal tracts (7 yeast species) |
| | | Alimentary canal (Hansenula silvicola) |
| | | Crop and gut (13 yeast species) |
| Apis mellifera | Hymenoptera: Apidae | Midguts (9 yeast genera) |
| Anthophora | Hymenoptera: Anthophoridae | |
| occidentalis | | |
| Nomia melanderi | Hymenoptera: Halictidae | |
| Halictus rubicundus | Hymenoptera: Halictidae | |
| Megachile rotundata | Hymenoptera: Megachilidae | |

Any number of fungal species may be targeted by the compositions or methods described herein. For example, in some instances, the modulating agent may target a single fungal species. In some instances, the modulating agent may target at least about any of 1, 2, 3, 4, 5, 6, 7, 8, 9, 10, 15, 20, 30, 40, 50, 60, 70, 80, 90, 100, 150, 200, 250, 500, or more distinct fungal species. In some instances, the modulating agent may target any one of about 1 to about 5, about 5 to about 10, about 10 to about 20, about 20 to about 50, about 50 to about 100, about 100 to about 200, about 200 to about 500, about 10 to about 50, about 5 to about 20, or about 10 to about 100 distinct fungal species. In some instances, the modulating agent may target at least about any of 1, 2, 3, 4, 5, 6, 7, 8, 9, 10, 15, 20, 30, 40, 50, 60, 70, 80, 90, 100, or more phyla, classes, orders, families, or genera of fungi.

In some instances, the modulating agent may increase a population of one or more fungi (e.g., pathogenic or parasitic fungi) by at least about any of 10%, 20%, 30%, 40%, 50%, 60%, 70%, 80%, 90% or more in the host in comparison to a host organism to which the modulating agent has not been administered. In some instances, the modulating agent may reduce the population of one or more fungi (e.g., symbiotic fungi) by at least about any of 10%, 20%, 30%, 40%, 50%, 60%, 70%, 80%, 90% or more in the host in comparison to a host organism to which the modulating agent has not been administered. In some instances, the modulating agent may eradicate the population of a fungi (e.g., symbiotic fungi) in the host. In some instances, the modulating agent may increase the level of one or more symbiotic fungi by at least about any of 10%, 20%, 30%, 40%, 50%, 60%, 70%, 80%, 90% or more in the host and/or may decrease the level of one or more symbiotic fungi by at least about any of 10%, 20%, 30%, 40%, 50%, 60%, 70%, 80%, 90% or more in the host in comparison to a host organism to which the modulating agent has not been administered.

In some instances, the modulating agent may alter the fungal diversity and/or fungal composition of the host. In some instances, the modulating agent may increase the fungal diversity in the host relative to a starting diversity by at least about any of 10%, 20%, 30%, 40%, 50%, 60%, 70%, 80%, 90%, or more in comparison to a host organism to which the modulating agent has not been administered. In some instances, the modulating agent may decrease the fungal diversity in the host relative to a starting diversity by at least about any of 10%, 20%, 30%, 40%, 50%, 60%, 70%, 80%, 90%, or more in comparison to a host organism to which the modulating agent has not been administered.

In some instances, the modulating agent may alter the function, activity, growth, and/or division of one or more fungi. For example, the modulating agent may alter the expression of one or more genes in the fungus. In some instances, the modulating agent may alter the function of one or more proteins in the fungus. In some instances, the modulating agent may alter the function of one or more cellular components in the fungus. In some instances, the modulating agent may kill the fungus.

Further, the target fungus may reside in one or more parts of the insect. In some instances, the fungus resides in or on one or more parts of the insect gut, including, e.g., the foregut, midgut, and/or hindgut. In some instances, the fungus lives extracellularly in the hemolymph, fat bodies or in specialized structures in the host.

Changes to the population of fungi in the host may be determined by any methods known in the art, such as microarray, polymerase chain reaction (PCR), real-time PCR, flow cytometry, fluorescence microscopy, transmission electron microscopy, fluorescence in situ hybridization (e.g., FISH), spectrophotometry, matrix-assisted laser desorption ionization-mass spectrometry (MALDI-MS), and DNA sequencing. In some instances, a sample of the host treated with a modulating agent is sequenced (e.g., by metagenomics sequencing) to determine the microbiome of the host after delivery or administration of the modulating agent. In some instances, a sample of a host that did not receive the modulating agent is also sequenced to provide a reference.

### III. Modulating Agents

The modulating agent of the methods and compositions provided herein may include a phage, a polypeptide, a small molecule, an antibiotic, a secondary metabolite, a bacterium, a fungus, or any combination thereof.

i. Phage

The modulating agent described herein may include a phage (e.g., a lytic phage or a non-lytic phage). In some instances, an effective concentration of any phage described herein may alter a level, activity, or metabolism of one or more microorganisms (as described herein, e.g., a Buchnera spp.) resident in a host described herein (e.g., an aphid), the modulation resulting in a decrease in the host's fitness (e.g., as outlined herein). In some instances, the modulating agent includes at least one phage selected from the order Tectiviridae, Myoviridae, Siphoviridae, Podoviridae, Caudovirales, Lipothrixviridae, Rudiviridae, or Ligamenvirales. In some instances, the composition includes at least one phage selected from the family Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Gluboloviridae, Guttaviridae, Inoviridae, Leviviridae, Microviridae, Plasmaviridae, and Tectiviridae. Further non-limiting examples of phages useful in the methods and compositions are listed in Table 3.

TABLE 3
Examples of Phage and Targeted Bacteria

| Phage and Accession # | Target bacteria | Target host |
|---|---|---|
| SA1(NC_027991), phiP68 | Staphylococcus | Apidae family |
| (NC_004679) | sp. | |
| WO (AB036666.1) | Wolbachia sp. | Aedes aegypt; Drosophila |
| | | melanogaster; |
| | | Plasmodium sp; |
| | | Teleogryllus taiwanemma; |
| | | Bombyx mori |
| KL1 (NC_018278), BcepNazgul | Burkholderia sp. | Riptortus sp.; Pyrrhocoris |
| (NC_005091) PhiE125 (NC_003309) | | apterus. |
| Fern (NC_028851), Xenia | Paenibacillus | bumble bees: Bombus |
| (NC_028837), HB10c2 (NC_028758) | larvae | sp.; honey bees: A. |
| | | mellifera |
| CP2 (NC_020205), XP10 (NC 004902), | Xanthomonas | Plebeina denoiti; Apidae |
| XP15 (NC_007024), phiL7 | sp. | family; Apis mellifera; |
| (NC_012742) | | Drosphilidae family; and |
| | | Chloropidae family |
| PP1 (NC_019542), PM1 (NC_023865) | Pectobacterium | Apidae family |
| | carotovorum | |
| | subsp. | |
| | carotovorum | |
| ϕRSA1 (NC_009382), | Ralstonia | Bombyx mori |
| ϕRSB1 (NC_011201), ϕRSL1 | solanacearum | |
| (NC_010811), RSM1 (NC 008574) | | |
| SF1(NC_028807) | Streptomyces | Philantus sp.; Trachypus |
| | scabies | sp |
| ECML-4 (NC_025446), ECML-117 | Escherichia coli | Apidae family; |
| (NC_025441), ECML-134 (NC_025449) | | Varroa destructor |
| SSP5(JX274646.1), SSP6 | Salmonella sp. | Drosphilidae family |
| (NC_004831), SFP10 (NC_016073), | | |
| F18SE (NC_028698) | | |
| γ (NC_001416), Bcp1 (NC_024137) | Bacillus sp. | Gypsy moth; Lymantria |
| | | dispar; Varroa destructor |
| Phi1 (NC_009821) | Enterococcus | Schistocerca gragaria |
| | sp. | |
| ϕKMV (NC_005045), | Pseudomonas | Lymantria dispar; Apidae |
| ϕEL(AJ697969.1), ϕKZ (NC_004629) | sp. | family |
| A2 (NC_004112), phig1e (NC_004305) | Lactobacilli sp. | Apidae family; Drosophila |
| | | family; Varroa destructor |
| KLPN1 (NC_028760) | Klebsiella sp | C. capitata |
| vB_AbaM_Acibel004 (NC_025462), | Acinetobacter | Schistocerca gragaria |
| vB_AbaP_Acibel007 (NC_025457) | sp. | |

In some instances, a modulating agent includes a lytic phage. Thus, after delivery of the lytic phage to a bacterial cell resident in the host, the phage causes lysis in the target bacterial cell. In some instances, the lytic phage targets and kills a bacterium resident in a host insect to decrease the fitness of the host. Alternatively or additionally, the phage of the modulating agent may be a non-lytic phage (also referred to as lysogenic or temperate phage). Thus, after delivery of the non-lytic phage to a bacterial cell resident in the host, the bacterial cell may remain viable and able to stably maintain expression of genes encoded in the phage genome. In some instances, a non-lytic phage is used to alter gene expression in a bacterium resident in a host insect to decrease the fitness of the host. In some instances, the modulating agent includes a mixture of lytic and non-lytic phage.

In certain instances, the phage is a naturally occurring phage. For example, a naturally occurring phage may be isolated from an environmental sample with a mixture of different phages. The naturally occurring phage may be isolated using methods known in the art to isolate, purify, and identify phage that target a particular microorganism (e.g., a bacterial endosymbiont in an insect host, e.g., a Buchnera spp. in aphids). Alternatively, in certain instances, the phage may be engineered based on a naturally occurring phage.

The modulating agent described herein may include phage with either a narrow or broad bacterial target range. In some instances, the phage has a narrow bacterial target range. In some instances, the phage is a promiscuous phage with a large bacterial target range. For example, the promiscuous phage may target at least about any of 5, 10, 20, 30, 40, 50, or more bacterium resident in the host. A phage with a narrow bacterial target range may target a specific bacterial strain in the host without affecting another, e.g., non-targeted, bacterium in the host. For example, the phage may target no more than about any of 50, 40, 30, 20, 10, 8, 6, 4, 2, or 1 bacterium resident in the host.

The compositions described herein may include any number of phage, such as at least about any one of 1, 2, 3, 4, 5, 10, 15, 20, 30, 40, 50, 100, or more phage. In some instances, the composition includes phage from one or more (e.g., 2, 3, 4, 5, 6, 7, 8, 9, 10, or more phage) families, one or more orders (e.g., 2, 3, 4, 5, 6, 7, 8, 9, 10, or more phage), or one or more species (e.g., 1, 2, 3, 4, 5, 10, 15, 20, 30, 40, 50, 100, or more phage). Compositions including one or more phage are also referred herein as “phage cocktails.” Phage cocktails are useful because they allow for targeting of a wider host range of bacteria. Furthermore, they allow for each bacterial strain (i.e. subspecies) to be targeted by multiple orthogonal phages, thereby preventing or significantly delaying the onset of resistance. In some instances, a cocktail includes multiple phages targeting one bacterial species. In some instances, a cocktail includes multiple phages targeting multiple bacterial species. In some instances, a one-phage “cocktail” includes a single promiscuous phage (i.e. a phage with a large host range) targeting many strains within a species.

Suitable concentrations of the phage in the modulating agent described herein depends on factors such as efficacy, survival rate, transmissibility of the phage, number of distinct phage, and/or lysin types in the compositions, formulation, and methods of application of the composition. In some instances, the phage is in a liquid or a solid formulation. In some instances, the concentration of each phage in any of the compositions described herein is at least about any of 10², 10³, 10⁴, 10⁵, 10⁶, 10⁷, 10⁸, 10⁹, 10¹⁰ or more pfu/ml. In some instances, the concentration of each phage in any of the compositions described herein is no more than about any of 10², 10³, 10⁴, 10⁵, 10⁶, 10⁷, 10⁸, 10⁹ pfu/ml. In some instances, the concentration of each phage in the composition is any of about 10² to about 10³, about 10³ to about 10⁴, about 10⁴ to about 10⁵, about 10⁵ to about 10⁶, about 10⁷ to about 10⁸, about 10⁸ to about 10⁹, about 10² to about 10⁴, about 10⁴ to about 10⁶, about 10⁶ to about 10⁹, or about 10³ to about 10⁸ pfu/ml. In some instances, wherein the composition includes at least two types of phages, the concentration of each type of the phages may be the same or different. For example, in some instances, the concentration of one phage in the cocktail is about 10⁸ pfu/ml and the concentration of a second phage in the cocktail is about 10⁶ pfu/ml.

A modulating agent including a phage as described herein can be contacted with the target host in an amount and for a time sufficient to: (a) reach a target level (e.g., a predetermined or threshold level) of phage concentration inside a target host; (b) reach a target level (e.g., a predetermined or threshold level) of phage concentration inside a target host gut; (c) reach a target level (e.g., a predetermined or threshold level) of phage concentration inside a target host bacteriocyte; (d) modulate the level, or an activity, of one or more microorganism (e.g., endosymbiont) in the target host; or/and (e) modulate fitness of the target host.

As illustrated by Examples 1-3 and 25, phages (e.g., one or more naturally occurring phage) can be used as modulating agents that target an endosymbiotic bacterium, such as a Buchnera spp., in an insect host, such as aphids, to decrease the fitness of the host (e.g., as outlined herein).

ii. Polypeptides

Numerous polypeptides (e.g., a bacteriocin, R-type bacteriocin, nodule C-rich peptide, antimicrobial peptide, lysin, or bacteriocyte regulatory peptide) may be used in the compositions and methods described herein. In some instances, an effective concentration of any peptide or polypeptide described herein may alter a level, activity, or metabolism of one or more microorganisms (as described herein, e.g., a Buchnera spp.) resident in a host (e.g., an aphid), the alteration resulting in a decrease in the host's fitness (e.g., as outlined herein). Polypeptides included herein may include naturally occurring polypeptides or recombinantly produced variants. For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide.

A modulating agent comprising a polypeptide as described herein can be contacted with the target host in an amount and for a time sufficient to: (a) reach a target level (e.g., a predetermined or threshold level) of concentration inside a target host; (b) reach a target level (e.g., a predetermined or threshold level) of concentration inside a target host gut; (c) reach a target level (e.g., a predetermined or threshold level) of concentration inside a target host bacteriocyte; (d) modulate the level, or an activity, of one or more microorganism (e.g., endosymbiont) in the target host; or/and (e) modulate fitness of the target host.

The polypeptide modulating agents discussed hereinafter, namely bacteriocins, lysins, antimicrobial peptides, nodule C-rich peptides, and bacteriocyte regulatory peptides, can be used to alter the level, activity, or metabolism of target microorganisms (e.g., Buchnera) as indicated in the section for decreasing the fitness of insects (e.g., aphids).

(a) Bacteriocins

The modulating agent described herein may include a bacteriocin. In some instances, the bacteriocin is naturally produced by Gram-positive bacteria, such as Pseudomonas, Streptomyces, Bacillus, Staphylococcus, or lactic acid bacteria (LAB, such as Lactococcus lactis). In some instances, the bacteriocin is naturally produced by Gram-negative bacteria, such as Hafnia alvei, Citrobacter freundii, Klebsiella oxytoca, Klebsiella pneumonia, Enterobacter cloacae, Serratia plymithicum, Xanthomonas campestris, Erwinia carotovora, Ralstonia solanacearum, or Escherichia coli. Exemplary bacteriocins include, but are not limited to, Class I-IV LAB antibiotics (such as lantibiotics), colicins, microcins, and pyocins. Non-limiting examples of bacteriocins are listed in Table 4.

TABLE 4
Examples of Bacteriocins

| Class | Name | Producer | Targets | Sequence |
|---|---|---|---|---|
| Class I | Nisin | Lactococcus | Active on Gram- | ITSISLCTPGCKT |
| | | lactis | positive bacteria: | GALMGCNMKTA |
| | | | Enterococcus, | TCHCSIHVSK |
| | | | Lactobacillus, | (SEQ ID NO: 42) |
| | | | Lactococcus, | |
| | | | Leuconostoc, | |
| | | | Listeria, | |
| | | | Clostridium | |
| | Epidermin | Staphylococcus | Gram-positive bacteria | IASKFICTPGCA |
| | | epidermis | | KTGSFNSYCC |
| | | | | (SEQ ID NO: 43) |
| Class II | | | | |
| Class II a | Pediocin PA-1 | Pediococcus | Pediococci, | KYYGNGVTCG |
| | | acidilactici | Lactobacilli, | KHSCSVDWGK |
| | | | Leuconostoc, | ATTCIINNGAMA |
| | | | Brochothrix | WATGGHQGNH |
| | | | thermosphacta, | KC |
| | | | Propionibacteria, | (SEQ ID NO: 44) |
| | | | Bacilli, | |
| | | | Enterococci, | |
| | | | Staphylococci, | |
| | | | Listeria clostridia, | |
| | | | Listeria | |
| | | | monocytogenes, | |
| | | | Listeria innocua | |
| Class II b | Enterocin P | Enterococcus | Lactobacillus sakei, | ATRSYGNGVYC |
| | | faecium | Enterococcus faecium | NNSKCWVNWG |
| | | | | EAKENIAGIVISG |
| | | | | WASGLAGMGH |
| | | | | (SEQ ID NO: 45) |
| Class II c | Lactococcin G | Streptococcus | Gram-positive bacteria | GTWDDIGQGIG |
| | | lactis | | RVAYWVGKAM |
| | | | | GNMSDVNQAS |
| | | | | RINRKKKH |
| | | | | (SEQ ID NO: 46) |
| Class II d | Lactacin-F | Lactobacillus | Lactobacilli, | NRWGDTVLSAA |
| | | johnsonii | Enterococcus faecalis | SGAGTGIKACK |
| | | | | SFGPWGMAICG |
| | | | | VGGAAIGGYFG |
| | | | | YTHN |
| | | | | (SEQ ID NO: 47) |
| Class III | | | | |
| Class III a | Enterocin AS- | Enterococcus | Broad spectrum: Gram | MAKEFGIPAAVA |
| | 48 | faecalis | positive and Gram | GTVLNVVEAGG |
| | | | negative bacteria. | WVTTIVSILTAV |
| | | | | GSGGLSLLAAA |
| | | | | GRESIKAYLKKE |
| | | | | IKKKGKRAVIAW |
| | | | | (SEQ ID NO: 48) |
| Class III b | Aureocin A70 | Staphylococcus | Broad spectrum: Gram | MSWLNFLKYIAK |
| | | aureus | positive and Gram | YGKKAVSAAWK |
| | | | negative bacteria. | YKGKVLEWLNV |
| | | | | GPTLEWVWQKL |
| | | | | KKIAGL |
| | | | | (SEQ ID NO: 49) |
| Class IV | Garvicin A | Lactococcus | Broad spectrum: Gram | IGGALGNALNGL |
| | | garvieae | positive and Gram | GTWANMMNGG |
| | | | negative bacteria. | GFVNQWQVYA |
| | | | | NKGKINQYRPY |
| | | | | (SEQ ID NO: 50) |
| Unclassified | Colicin V | Escherichia coli | Active against | MRTLTLNELDS |
| | | | Escherichia coli (also | VSGGASGRDIA |
| | | | closely related | MAIGTLSGQFV |
| | | | bacteria), | AGGIGAAAGGV |
| | | | Enterobacteriaceae | AGGAIYDYAST |
| | | | | HKPNPAMSPSG |
| | | | | LGGTIKQKPEGI |
| | | | | PSEAWNYAAGR |
| | | | | LCNWSPNNLSD |
| | | | | VCL |
| | | | | (SEQ ID NO: 51) |

In some instances, the bacteriocin is a colicin, a pyocin, or a microcin produced by Gram-negative bacteria. In some instances, the bacteriocin is a colicin. The colicin may be a group A colicin (e.g., uses the Tol system to penetrate the outer membrane of a target bacterium) or a group B colicin (e.g., uses the Ton system to penetrate the outer membrane of a target bacterium). In some instances, the bacteriocin is a microcin. The microcin may be a class I microcin (e.g., <5 kDa, has post-translational modifications) or a class II microcin (e.g., 5-10 kDa, with or without post-translational modifications). In some instances, the class II microcin is a class IIa microcin (e.g., requires more than one genes to synthesize and assemble functional peptides) or a class IIb microcin (e.g., linear peptides with or without post-translational modifications at C-terminus). In some instances, the bacteriocin is a pyocin. In some instances, the pyocin is an R-pyocin, F-pyocin, or S-pyocin.

In some instances, the bacteriocin is a class I, class II, class III, or class IV bacteriocin produced by Gram-positive bacteria. In some instances, the modulating agent includes a Class I bacteriocin (e.g., lanthionine-containing antibiotics (lantibiotics) produced by a Gram-positive bacteria). The class I bacteriocins or lantibiotic may be a low molecular weight peptide (e.g., less than about 5 kDa) and may possess post-translationally modified amino acid residues (e.g., lanthionine, β-methyllanthionine, or dehydrated amino acids).

In some instances, the bacteriocin is a Class II bacteriocin (e.g., non-lantibiotics produced by Gram-positive bacteria). Many are positively charged, non-lanthionine-containing peptides, which unlike lantibiotics, do not undergo extensive post-translational modification. The Class II bacteriocin may belong to one of the following subclasses: “pediocin-like” bacteriocins (e.g., pediocin PA-1 and carnobacteriocin X (Class IIa)); two-peptide bacteriocins (e.g., lactacin F and ABP-118 (Class IIb)); circular bacteriocins (e.g., carnocyclin A and enterocin AS-48 (Class IIc)); or unmodified, linear, non-pediocin-like bacteriocins (e.g., epidermicin N|01 and lactococcin A (Class IId)).

In some instances, the bacteriocin is a Class III bacteriocin (e.g., produced by Gram-positive bacteria). Class III bacteriocins may have a molecular weight greater than 10 kDa and may be heat unstable proteins. The Class III bacteriocins can be further subdivided into Group IIIA and Group IIIB bacteriocins. The Group IIIA bacteriocins include bacteriolytic enzymes which kill sensitive strains by lysis of the cell well, such as Enterolisin A. Group IIIB bacteriocins include non-lytic proteins, such as Caseicin 80, Helveticin J, and lactacin B.

In some instances, the bacteriocin is a Class IV bacteriocin (e.g., produced by Gram-positive bacteria). Class IV bacteriocins are a group of complex proteins, associated with other lipid or carbohydrate moieties, which appear to be required for activity. They are relatively hydrophobic and heat stable. Examples of Class IV bacteriocins leuconocin S, lactocin 27, and lactocin S.

In some instances, the bacteriocin is an R-type bacteriocin. R-type bacteriocins are contractile bacteriocidal protein complexes. Some R-type bacteriocins have a contractile phage-tail-like structure. The C-terminal region of the phage tail fiber protein determines target-binding specificity. They may attach to target cells through a receptor-binding protein, e.g., a tail fiber. Attachment is followed by sheath contraction and insertion of the core through the envelope of the target bacterium. The core penetration results in a rapid depolarization of the cell membrane potential and prompt cell death. Contact with a single R-type bacteriocin particle can result in cell death. An R-type bacteriocin, for example, may be thermolabile, mild acid resistant, trypsin resistant, sedimentable by centrifugation, resolvable by electron microscopy, or a combination thereof. Other R-type bacteriocins may be complex molecules including multiple proteins, polypeptides, or subunits, and may resemble a tail structure of bacteriophages of the myoviridae family. In naturally occurring R-type bacteriocins, the subunit structures may be encoded by a bacterial genome, such as that of C. difficile or P. aeruginosa and form R-type bacteriocins to serve as natural defenses against other bacteria. In some instances, the R-type bacteriocin is a pyocin. In some instances, the pyocin is an R-pyocin, F-pyocin, or S-pyocin.

In some instances, the bacteriocin is a functionally active variant of the bacteriocins described herein. In some instances, the variant of the bacteriocin has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a bacteriocin described herein or a naturally occurring bacteriocin.

In some instances, the bacteriocin may be bioengineered, according to standard methods, to modulate their bioactivity, e.g., increase or decrease or regulate, or to specify their target microorganisms. In other instances, the bacteriocin is produced by the translational machinery (e.g. a ribosome, etc.) of a microbial cell. In some instances, the bacteriocin is chemically synthesized. Some bacteriocins can be derived from a polypeptide precursor. The polypeptide precursor can undergo cleavage (e.g., processing by a protease) to yield the polypeptide of the bacteriocin itself. As such, in some instances, the bacteriocin is produced from a precursor polypeptide. In some other instances, the bacteriocin includes a polypeptide that has undergone post-translational modifications, for example, cleavage, or the addition of one or more functional groups.

The bacteriocins described herein may be formulated in a composition for any of the uses described herein. The compositions disclosed herein may include any number or type (e.g., classes) of bacteriocins, such as at least about any one of 1 bacteriocin, 2, 3, 4, 5, 10, 15, 20, 30, 40, 50, 100, or more bacteriocins. Suitable concentrations of each bacteriocin in the compositions described herein depends on factors such as efficacy, stability of the bacteriocin, number of distinct bacteriocin types in the compositions, formulation, and methods of application of the composition. In some instances, each bacteriocin in a liquid composition is from about 0.01 ng/ml to about 100 mg/mL. In some instances, each bacteriocin in a solid composition is from about 0.01 ng/g to about 100 mg/g. In some instances, wherein the composition includes at least two types of bacteriocins, the concentration of each type of the bacteriocins may be the same or different. In some instances, the bacteriocin is provided in a composition including a bacterial cell that secretes the bacteriocin. In some instances, the bacteriocin is provided in a composition including a polypeptide (e.g., a polypeptide isolated from a bacterial cell).

Bacteriocins may neutralize (e.g., kill) at least one microorganism other than the individual bacterial cell in which the polypeptide is made, including cells clonally related to the bacterial cell and other microbial cells. As such, a bacterial cell may exert cytotoxic or growth-inhibiting effects on a plurality of microbial organisms by secreting bacteriocins. In some instances, the bacteriocin targets and kills one or more species of bacteria resident in the host via cytoplasmic membrane pore formation, cell wall interference (e.g., peptidoglycanase activity), or nuclease activity (e.g., DNase activity, 16S rRNase activity, or tRNase activity).

In some instances, the bacteriocin has a neutralizing activity. Neutralizing activity of bacteriocins may include, but is not limited to, arrest of microbial reproduction, or cytotoxicity. Some bacteriocins have cytotoxic activity, and thus can kill microbial organisms, for example bacteria, yeast, algae, and the like. Some bacteriocins can inhibit the reproduction of microbial organisms, for example bacteria, yeast, algae, and the like, for example by arresting the cell cycle.

In some instances, the bacteriocin has killing activity. The killing mechanism of bacteriocins is specific to each group of bacteriocins. In some instances, the bacteriocin has narrow-spectrum bioactivity. Bacteriocins are known for their very high potency against their target strains. Some bacteriocin activity is limited to strains that are closely related to the bacteriocin producer strain (narrow-spectrum bioactivity). In some instances, the bacteriocin has broad-spectrum bioactivity against a wide range of genera.

In some instances, bacteriocins interact with a receptor molecule or a docking molecule on the target bacterial cell membrane. For example, nisin is extremely potent against its target bacterial strains, showing antimicrobial activity even at a single-digit nanomolar concentration. The nisin molecule has been shown to bind to lipid II, which is the main transporter of peptidoglycan subunits from the cytoplasm to the cell wall

In some instances, the bacteriocin has anti-fungal activity. A number of bacteriocins with anti-yeast or anti-fungal activity have been identified. For example, bacteriocins from Bacillus have been shown to have neutralizing activity against some yeast strains (see, for example, Adetunji and Olaoye, Malaysian Journal of Microbiology 9:130-13, 2013). In another example, an Enterococcus faecalis peptide has been shown to have neutralizing activity against Candida species (see, for example, Shekh and Roy, BMC Microbiology 12:132, 2012). In another example, bacteriocins from Pseudomonas have been shown to have neutralizing activity against fungi, such as Curvularia lunata, Fusarium species, Helminthosporium species, and Biopolaris species (see, for example, Shalani and Srivastava, The Internet Journal of Microbiology Volume 5 Number 2, 2008). In another example, botrycidin AJ1316 and alirin B1 from B. subtilis have been shown to have antifungal activities.

A modulating agent including a bacteriocin as described herein can be contacted with the target host in an amount and for a time sufficient to: (a) reach a target level (e.g., a predetermined or threshold level) of bacteriocin concentration inside a target host; (b) reach a target level (e.g., a predetermined or threshold level) of bacteriocin concentration inside a target host gut; (c) reach a target level (e.g., a predetermined or threshold level) of bacteriocin concentration inside a target host bacteriocyte; (d) modulate the level, or an activity, of one or more microorganism (e.g., endosymbiont) in the target host; or/and (e) modulate fitness of the target host.

As illustrated by Examples 4, 5, and 13, bacteriocins (e.g., colA or nisin) can be used as modulating agents that target an endosymbiotic bacterium, such as a Buchnera spp., in an insect host, such as aphids, to decrease the fitness of the host (e.g., as outlined herein).

(b) Lysins

The modulating agent described herein may include a lysin (e.g., also known as a murein hydrolase or peptidoglycan autolysin). Any lysin suitable for inhibiting a bacterium resident in the host may be used. In some instances, the lysin is one that can be naturally produced by a bacterial cell. In some instances, the lysin is one that can be naturally produced by a bacteriophage. In some instances, the lysin is obtained from a phage that inhibits a bacterium resident in the host. In some instances, the lysin is engineered based on a naturally occurring lysin. In some instances, the lysin is engineered to be secreted by a host bacterium, for example, by introducing a signal peptide to the lysin. In some instances, the lysin is used in combination with a phage holin. In some instances, a lysin is expressed by a recombinant bacterium host that is not sensitive to the lysin. In some instances, the lysin is used to inhibit a Gram-positive or Gram-negative bacterium resident in the host.

The lysin may be any class of lysin and may have one or more substrate specificities. For example, the lysin may be a glycosidase, an endopeptidase, a carboxypeptidase, or a combination thereof. In some instances, the lysin cleaves the β-1-4 glycosidic bond in the sugar moiety of the cell wall, the amide bond connecting the sugar and peptide moieties of the bacterial cell wall, and/or the peptide bonds between the peptide moieties of the cell wall. The lysin may belong to one or more specific lysin groups, depending on the cleavage site within the peptidoglycan. In some instances, the lysin is a N-acetyl-β-D-muramidase (e.g., lysozyme), lytic transglycosylase, N-acetyl-β-D-glucosaminidase, N-acetylmuramyl-L-alanine amidase, L,D-endopeptidase, D,D-endopeptidase, D,D-carboxypeptidase, L,D-carboxypeptidase, or L,D-transpeptidase. Non-limiting examples of lysins and their activities are listed in Table 5.

TABLE 5
Examples of Lysins

| Target Bacteria | Producer | Lysins | Activity | Sequence |
|---|---|---|---|---|
| S. pneumoniae | Cp1 | Cpl-1 | Muramidase | MVKKNDLFVDVSSHN |
| | | | | GYDITGILEQMGTTNTI |
| | | | | IKISESTTYLNPCLSAQ |
| | | | | VEQSNPIGFYHFARFG |
| | | | | GDVAEAEREAQFFLD |
| | | | | NVPMQVKYLVLDYED |
| | | | | DPSGDAQANTNACLR |
| | | | | FMQMIADAGYKPIYYS |
| | | | | YKPFTHDNVDYQQILA |
| | | | | QFPNSLWIAGYGLND |
| | | | | GTANFEYFPSMDGIR |
| | | | | WWQYSSNPFDKNIVL |
| | | | | LDDEEDDKPKTAGTW |
| | | | | KQDSKGWWFRRNNG |
| | | | | SFPYNKWEKIGGVWY |
| | | | | YFDSKGYCLTSEWLK |
| | | | | DNEKWYYLKDNGAM |
| | | | | ATGWVLVGSEWYYM |
| | | | | DDSGAMVTGWVKYK |
| | | | | NNWYYMTNERGNMV |
| | | | | SNEFIKSGKGWYFMN |
| | | | | TNGELADNPSFTKEP |
| | | | | DGLITVA |
| | | | | (SEQ ID NO: 52) |
| S. pneumoniae | Dp-1 | Pal | Amidase | MGVDIEKGVAWMQA |
| | | | | RKGRVSYSMDFRDGP |
| | | | | DSYDCSSSMYYALRS |
| | | | | AGASSAGWAVNTEY |
| | | | | MHAWLIENGYELISEN |
| | | | | APWDAKRGDIFIWGR |
| | | | | KGASAGAGGHTGMFI |
| | | | | DSDNIIHCNYAYDGIS |
| | | | | VNDHDERWYYAGQP |
| | | | | YYYVYRLTNANAQPA |
| | | | | EKKLGWQKDATGFW |
| | | | | YARANGTYPKDEFEYI |
| | | | | EENKSWFYFDDQGY |
| | | | | MLAEKVVLKHTDGNW |
| | | | | YWFDRDGYMATSWK |
| | | | | RIGESWYYFNRDGSM |
| | | | | VTGWIKYYDNWYYCD |
| | | | | ATNGDMKSNAFIRYN |
| | | | | DGWYLLLPDGRLADK |
| | | | | PQFTVEPDGLITAKV |
| | | | | (SEQ ID NO: 53) |
| S. pyogenes | C1 | C1 | Amidase | N/A |
| B. anthracis | γ | PlyG | Amidase | MEIQKKLVDPSKYGTK |
| | | | | CPYTMKPKYITVHNTY |
| | | | | NDAPAENEVSYMISN |
| | | | | NNEVSFHIAVDDKKAI |
| | | | | QGIPLERNAWACGDG |
| | | | | NGSGNRQSISVEICYS |
| | | | | KSGGDRYYKAEDNAV |
| | | | | DVVRQLMSMYNIPIEN |
| | | | | VRTHQSWSGKYCPH |
| | | | | RMLAEGRWGAFIQKV |
| | | | | KNGNVATTSPTKQNII |
| | | | | QSGAFSPYETPDVMG |
| | | | | ALTSLKMTADFILQSD |
| | | | | GLTYFISKPTSDAQLK |
| | | | | AMKEYLDRKGWWYE |
| | | | | VK |
| | | | | (SEQ ID NO: 54) |
| B. anthracis | Ames | PlyPH | Amidase | N/A |
| | prophage | | | |
| E. faecalis and E. faecium | Phi1 | PlyV12 | Amidase | N/A |
| S. aureus | φMR11 | MV-L | Endopeptidase | MQAKLTKKEFIEWLKT |
| | | | and amidase | SEGKQFNVDLWYGFQ |
| | | | | CFDYANAGWKVLFGL |
| | | | | LLKGLGAKDIPFANNF |
| | | | | DGLATVYQNTPDFLA |
| | | | | QPGDMVVFGSNYGA |
| | | | | GYGHVAWVIEATLDYII |
| | | | | VYEQNWLGGGWTDRI |
| | | | | EQPGWGWEKVTRRQ |
| | | | | HAYDFPMWFIRPNFK |
| | | | | SETAPRSIQSPTQASK |
| | | | | KETAKPQPKAVELKIIK |
| | | | | DVVKGYDLPKRGGNP |
| | | | | KGIVIHNDAGSKGATA |
| | | | | EAYRNGLVNAPLSRL |
| | | | | EAGIAHSYVSGNTVW |
| | | | | QALDESQVGWHTAN |
| | | | | QLGNKYYYGIEVCQS |
| | | | | MGADNATFLKNEQAT |
| | | | | FQECARLLKKWGLPA |
| | | | | NRNTIRLHNEFTSTSC |
| | | | | PHRSSVLHTGFDPVT |
| | | | | RGLLPEDKQLQLKDY |
| | | | | FIKQIRVYMDGKIPVAT |
| | | | | VSNESSASSNTVKPV |
| | | | | ASAWKRNKYGTYYME |
| | | | | ENARFTNGNQPITVRK |
| | | | | IGPFLSCPVAYQFQPG |
| | | | | GYCDYTEVMLQDGHV |
| | | | | WVGYTWEGQRYYLPI |
| | | | | RTWNGSAPPNQILGD |
| | | | | LWGEIS |
| | | | | (SEQ ID NO: 55) |
| S. pyogenes | C1 | PlyC | Amidase | N/A |
| S. agalactiae | B30 | GBS lysin | Muramidase and | MVINIEQAIAWMASRK |
| | | | endopeptidase | GKVTYSMDYRNGPSS |
| | | | | YDCSSSVYFALRSAG |
| | | | | ASDNGWAVNTEYEHD |
| | | | | WLIKNGYVLIAENTNW |
| | | | | NAQRGDIFIWGKRGA |
| | | | | SAGAFGHTGMFVDPD |
| | | | | NIIHCNYGYNSITVNN |
| | | | | HDEIWGYNGQPYVYA |
| | | | | YRYSGKQSNAKVDNK |
| | | | | SVVSKFEKELDVNTPL |
| | | | | SNSNMPYYEATISEDY |
| | | | | YVESKPDVNSTDKELL |
| | | | | VAGTRVRVYEKVKGW |
| | | | | ARIGAPQSNQWVEDA |
| | | | | YLIDATDM |
| | | | | (SEQ ID NO: 56) |
| S. aureus | P68 | Lys16 | Endopeptidase | N/A |
| S. aureus | K | LysK | Amidase and | MAKTQAEINKRLDAYA |
| | | | endopeptidase | KGTVDSPYRVKKATS |
| | | | | YDPSFGVMEAGAIDA |
| | | | | DGYYHAQCQDLITDY |
| | | | | VLWLTDNKVRTWGNA |
| | | | | KDQIKQSYGTGFKIHE |
| | | | | NKPSTVPKKGWIAVFT |
| | | | | SGSYEQWGHIGIVYD |
| | | | | GGNTSTFTILEQNWN |
| | | | | GYANKKPTKRVDNYY |
| | | | | GLTHFIEIPVKAGTTVK |
| | | | | KETAKKSASKTPAPKK |
| | | | | KATLKVSKNHINYTMD |
| | | | | KRGKKPEGMVIHNDA |
| | | | | GRSSGQQYENSLANA |
| | | | | GYARYANGIAHYYGS |
| | | | | EGYVWEAIDAKNQIA |
| | | | | WHTGDGTGANSGNF |
| | | | | RFAGIEVCQSMSASD |
| | | | | AQFLKNEQAVFQFTA |
| | | | | EKFKEWGLTPNRKTV |
| | | | | RLHMEFVPTACPHRS |
| | | | | MVLHTGFNPVTQGRP |
| | | | | SQAIMNKLKDYFIKQIK |
| | | | | NYMDKGTSSSTVVKD |
| | | | | GKTSSASTPATRPVT |
| | | | | GSWKKNQYGTWYKP |
| | | | | ENATFVNGNQPIVTRI |
| | | | | GSPFLNAPVGGNLPA |
| | | | | GATIVYDEVCIQAGHI |
| | | | | WIGYNAYNGNRVYCP |
| | | | | VRTCQGVPPNQIPGV |
| | | | | AWGVFK |
| | | | | (SEQ ID NO: 57) |
| L. monocytogenes | A118 | Ply118 | Amidase | MTSYYYSRSLANVNK |
| | | | | LADNTKAAARKLLDW |
| | | | | SESNGIEVLIYETIRTK |
| | | | | EQQAANVNSGASQT |
| | | | | MRSYHLVGQALDFVM |
| | | | | AKGKTVDWGAYRSDK |
| | | | | GKKFVAKAKSLGFEW |
| | | | | GGDWSGFVDNPHLQ |
| | | | | FNYKGYGTDTFGKGA |
| | | | | STSNSSKPSADTNTN |
| | | | | SLGLVDYMNLNKLDS |
| | | | | SFANRKKLATSYGIKN |
| | | | | YSGTATQNTTLLAKLK |
| | | | | AGKPHTPASKNTYYT |
| | | | | ENPRKVKTLVQCDLY |
| | | | | KSVDFTTKNQTGGTF |
| | | | | PPGTVFTISGMGKTK |
| | | | | GGTPRLKTKSGYYLT |
| | | | | ANTKFVKKI |
| | | | | (SEQ ID NO: 58) |
| L. monocytogenes | A511 | Ply511 | Amidase | MVKYTVENKIIAGLPK |
| | | | | GKLKGANFVIAHETAN |
| | | | | SKSTIDNEVSYMTRN |
| | | | | WKNAFVTHFVGGGG |
| | | | | RVVQVANVNYVSWG |
| | | | | AGQYANSYSYAQVEL |
| | | | | CRTSNATTFKKDYEV |
| | | | | YCQLLVDLAKKAGIPIT |
| | | | | LDSGSKTSDKGIKSHK |
| | | | | WVADKLGGTTHQDPY |
| | | | | AYLSSWGISKAQFAS |
| | | | | DLAKVSGGGNTGTAP |
| | | | | AKPSTPAPKPSTPSTN |
| | | | | LDKLGLVDYMNAKKM |
| | | | | DSSYSNRDKLAKQYGI |
| | | | | ANYSGTASQNTTLLSK |
| | | | | IKGGAPKPSTPAPKPS |
| | | | | TSTAKKIYFPPNKGNW |
| | | | | SVYPTNKAPVKANAIG |
| | | | | AINPTKFGGLTYTIQK |
| | | | | DRGNGVYEIQTDQFG |
| | | | | RVQVYGAPSTGAVIKK |
| | | | | (SEQ ID NO: 59) |
| L. monocytogenes | A500 | Ply500 | Endopeptidase | MALTEAWLIEKANRKL |
| | | | | NAGGMYKITSDKTRN |
| | | | | VIKKMAKEGIYLCVAQ |
| | | | | GYRSTAEQNALYAQG |
| | | | | RTKPGAIVTNAKGGQ |
| | | | | SNHNYGVAVDLCLYT |
| | | | | NDGKDVIWESTTSRW |
| | | | | KKVVAAMKAEGFKWG |
| | | | | GDWKSFKDYPHFELC |
| | | | | DAVSGEKIPAATQNTN |
| | | | | TNSNRYEGKVIDSAPL |
| | | | | LPKMDFKSSPFRMYK |
| | | | | VGTEFLVYDHNQYVVY |
| | | | | KTYIDDKLYYMYKSFC |
| | | | | DVVAKKDAKGRIKVRI |
| | | | | KSAKDLRIPVWNNIKL |
| | | | | NSGKIKWYAPNVKLA |
| | | | | WYNYRRGYLELWYP |
| | | | | NDGWYYTAEYFLK |
| | | | | (SEQ ID NO: 60) |
| S. pneumoniae | φDp-1 | Pal, S | Endopeptidase | N/A |
| | | | and amidase | |
| S. agalactiae | LambdaSa1 | LambdaSa1 | Glycosidase | MVINIEQAIAWMASRK |
| | prophage | | | GKVTYSMDYRNGPSS |
| | | | | YDCSSSVYFALRSAG |
| | | | | ASDNGWAVNTEYEHD |
| | | | | WLIKNGYVLIAENTNW |
| | | | | NAQRGDIFIWGKRGA |
| | | | | SAGAFGHTGMFVDPD |
| | | | | NIIHCNYGYNSITVNN |
| | | | | HDEIWGYNGQPYVYA |
| | | | | YRYARKQSNAKVDNQ |
| | | | | SVVSKFEKELDVNTPL |
| | | | | SNSNMPYYEATISEDY |
| | | | | YVESKPDVNSTDKELL |
| | | | | VAGTRVRVYEKVKGW |
| | | | | ARIGAPQSNQWVEDA |
| | | | | YLIDATDM |
| | | | | (SEQ ID NO: 61) |
| S. agalactiae | LambdaSa2 | LambdaSa2 | Glycosidase and | MEINTEIAIAWMSARQ |
| | prophage | | endopeptidase | GKVSYSMDYRDGPNS |
| | | | | YDCSSSVYYALRSAG |
| | | | | ASSAGWAVNTEYMH |
| | | | | DWLIKNGYELIAENVD |
| | | | | WNAVRGDIAIWGMRG |
| | | | | HSSGAGGHVVMFIDP |
| | | | | ENIIHCNWANNGITVN |
| | | | | NYNQTAAASGWMYC |
| | | | | YVYRLKSGASTQGKS |
| | | | | LDTLVKETLAGNYGN |
| | | | | GEARKAVLGNQYEAV |
| | | | | MSVINGKTTTNQKTVD |
| | | | | QLVQEVIAGKHGNGE |
| | | | | ARKKSLGSQYDAVQK |
| | | | | RVTELLKKQPSEPFKA |
| | | | | QEVNKPTETKTSQTEL |
| | | | | TGQATATKEEGDLSF |
| | | | | NGTILKKAVLDKILGNC |
| | | | | KKHDILPSYALTILHYE |
| | | | | GLWGTSAVGKADNN |
| | | | | WGGMTWTGQGNRPS |
| | | | | GVTVTQGSARPSNEG |
| | | | | GHYMHYASVDDFLTD |
| | | | | WFYLLRAGGSYKVSG |
| | | | | AKTFSEAIKGMFKVGG |
| | | | | AVYDYAASGFDSYIVG |
| | | | | ASSRLKAIEAENGSLD |
| | | | | KFDKATDIGDGSKDKI |
| | | | | DITIEGIEVTINGITYEL |
| | | | | TKKPV |
| | | | | (SEQ ID NO: 62) |
| S. uberis | (ATCC700407) | Ply700 | Amidase | MTDSIQEMRKLOSIPV |
| | prophage | | | RYDMGDRYGNDADR |
| | | | | DGRIEMDCSSAVSKA |
| | | | | LGISMTNNTETLQQAL |
| | | | | PAIGYGKIHDAVDGTF |
| | | | | DMQAYDVIIWAPRDG |
| | | | | SSSLGAFGHVLIATSP |
| | | | | TTAIHCNYGSDGITEN |
| | | | | DYNYIWEINGRPREIV |
| | | | | FRKGVTQTQATVTSQ |
| | | | | FERELDVNARLTVSDK |
| | | | | PYYEATLSEDYYVEA |
| | | | | GPRIDSQDKELIKAGT |
| | | | | RVRVYEKLNGWSRIN |
| | | | | HPESAQWVEDSYLVD |
| | | | | ATEM |
| | | | | (SEQ ID NO: 63) |
| S. suis | SMP | LySMP | Glycosidase and | N/A |
| | | | endopeptidase | |
| B. anthracis | Bcp1 | PlyB | Muramidase | N/A |
| S. aureus | Phi11 and | Phi11 lysin | Amidase and | MQAKLTKNEFIEWLKT |
| | Phi12 | | endopeptidase | SEGKQFNVDLWYGFQ |
| | | | | CFDYANAGWKVLFGL |
| | | | | LLKGLGAKDIPFANNF |
| | | | | DGLATVYQNTPDFLA |
| | | | | QPGDMVVFGSNYGA |
| | | | | GYGHVAWVIEATLDYII |
| | | | | VYEQNWLGGGWTDG |
| | | | | IEQPGWGWEKVTRR |
| | | | | QHAYDFPMWFIRPNF |
| | | | | KSETAPRSVQSPTQA |
| | | | | PKKETAKPQPKAVELK |
| | | | | IIKDVVKGYDLPKRGS |
| | | | | NPKGIVIHNDAGSKGA |
| | | | | TAEAYRNGLVNAPLS |
| | | | | RLEAGIAHSYVSGNTV |
| | | | | WQALDESQVGWHTA |
| | | | | NQIGNKYYYGIEVCQS |
| | | | | MGADNATFLKNEQAT |
| | | | | FQECARLLKKWGLPA |
| | | | | NRNTIRLHNEFTSTSC |
| | | | | PHRSSVLHTGFDPVT |
| | | | | RGLLPEDKRLQLKDYF |
| | | | | IKQIRAYMDGKIPVATV |
| | | | | SNESSASSNTVKPVA |
| | | | | SAWKRNKYGTYYMEE |
| | | | | SARFTNGNQPITVRKV |
| | | | | GPFLSCPVGYQFQPG |
| | | | | GYCDYTEVMLQDGHV |
| | | | | WVGYTWEGQRYYLPI |
| | | | | RTWNGSAPPNQILGD |
| | | | | LWGEIS |
| | | | | (SEQ ID NO: 64) |
| S. aureus | φH5 | LysH5 | Amidase and | MQAKLTKKEFIEWLKT |
| | | | endopeptidase | SEGKQYNADGWYGF |
| | | | | QCFDYANAGWKALFG |
| | | | | LLLKGVGAKDIPFANN |
| | | | | FDGLATVYQNTPDFLA |
| | | | | QPGDMVVFGSNYGA |
| | | | | GYGHVAWVIEATLDYII |
| | | | | VYEQNWLGGGWTDG |
| | | | | VQQPGSGWEKVTRR |
| | | | | QHAYDFPMWFIRPNF |
| | | | | KSETAPRSVQSPTQA |
| | | | | SKKETAKPQPKAVELK |
| | | | | IIKDVVKGYDLPKRGS |
| | | | | NPNFIVIHNDAGSKGA |
| | | | | TAEAYRNGLVNAPLS |
| | | | | RLEAGIAHSYVSGNTV |
| | | | | WQALDESQVGWHTA |
| | | | | NQIGNKYGYGIEVCQS |
| | | | | MGADNATFLKNEQAT |
| | | | | FQECARLLKKWGLPA |
| | | | | NRNTIRLHNEFTSTSC |
| | | | | PHRSSVLHTGFDPVT |
| | | | | RGLLPEDKRLQLKDYF |
| | | | | IKQIRAYMDGKIPVATV |
| | | | | SNDSSASSNTVKPVA |
| | | | | SAWKRNKYGTYYMEE |
| | | | | SARFTNGNQPITVRKV |
| | | | | GPFLSCPVGYQFQPG |
| | | | | GYCDYTEVMLQDGHV |
| | | | | WVGYTWEGQRYYLPI |
| | | | | RTVVNGSAPPNQILGD |
| | | | | LWGEIS |
| | | | | (SEQ ID NO: 65) |
| S. warneri | φWMY | LysWMY | Amidase and | MKTKAQAKSWINSKIG |
| | | | endopeptidase | KGIDWDGMYGYQCM |
| | | | | DEAVDYIHHVTDGKVT |
| | | | | MWGNAIDAPKNNFQG |
| | | | | LCTVYTNTPEFRPAYG |
| | | | | DVIVWSYGTFATYGHI |
| | | | | AIVVNPDPYGDLQYIT |
| | | | | VLEQNWNGNGIYKTE |
| | | | | FATIRTHDYTGVSHFI |
| | | | | RPKFADEVKETAKTV |
| | | | | NKLSVQKKIVTPKNSV |
| | | | | ERIKNYVKTSGYINGE |
| | | | | HYELYNRGHKPKGVVI |
| | | | | HNTAGTASATQEGQR |
| | | | | LTNMTFQQLANGVAH |
| | | | | VYIDKNTIYETLPEDRI |
| | | | | AWHVAQQYGNTEFY |
| | | | | GIEVCGSRNTDKEQFL |
| | | | | ANEQVAFQEAARRLK |
| | | | | SWGMRANRNTVRLH |
| | | | | HTFSSTECPDMSMLL |
| | | | | HTGYSMKNGKPTQDI |
| | | | | TNKCADYFMKQINAYI |
| | | | | DGKQPTSTVVGSSSS |
| | | | | NKLKAKNKDKSTGWN |
| | | | | TNEYGTLWKKEHATF |
| | | | | TCGVRQGIVTRTTGPF |
| | | | | TSCPQAGVLYYGQSV |
| | | | | NYDTVCKQDGYVWIS |
| | | | | WTTSDGYDVWMPIRT |
| | | | | WDRSTDKVSEIWGTIS |
| | | | | (SEQ ID NO: 66) |
| Streptococci (GBS) | φNCTC | PlyGBS | Muramidase and | MATYQEYKSRSNGNA |
| | 11261 | | endopeptidase | YDIDGSFGAQCWDGY |
| | | | | ADYCKYLGLPYANCT |
| | | | | NTGYARDIWEQRHEN |
| | | | | GILNYFDEVEVMQAG |
| | | | | DVAIFMVVDGVTPYSH |
| | | | | VAIFDSDAGGGYGWF |
| | | | | LGQNQGGANGAYNIV |
| | | | | KIPYSATYPTAFRPKV |
| | | | | FKNAVTVTGNIGLNKG |
| | | | | DYFIDVSAYQQADLTT |
| | | | | TCQQAGTTKTIIKVSE |
| | | | | SIAWLSDRHQQQANT |
| | | | | SDPIGYYHFGRFGGD |
| | | | | SALAQREADLFLSNLP |
| | | | | SKKVSYLVIDYEDSAS |
| | | | | ADKQANTNAVIAFMD |
| | | | | KIASAGYKPIYYSYKPF |
| | | | | TLNNIDYQKIIAKYPNSI |
| | | | | WIAGYPDYEVRTEPL |
| | | | | WEFFPSMDGVRWWQ |
| | | | | FTSVGVAGGLDKNIVL |
| | | | | LADDSSKMDIPKVDKP |
| | | | | QELTFYQKLATNTKLD |
| | | | | NSNVPYYEATLSTDYY |
| | | | | VESKPNASSADKEFIK |
| | | | | AGTRVRVYEKVNGWS |
| | | | | RINHPESAQWVEDSY |
| | | | | LVNATDM |
| | | | | (SEQ ID NO: 67) |
| C. perfringens | φ3626 | Ply3626 | Amidase | N/A |
| C. difficile | φCD27 | CD27 lysin | Amidase | N/A |
| E. faecalis | φ1 | PlyV12 | Amidase | N/A |
| A. naeslundii | φAv-1- | Av-1 lysin | Putative amidase/ | N/A |
| | | | muramidase | |
| L. gasseri | φgaY | LysgaY | Muramidase | N/A |
| S. aureus | φSA4 | LysSA4 | Amidase and | N/A |
| | | | endopeptidase | |
| S. haemolyticus | φSH2 | SH2 | Amidase and | N/A |
| | | | endopeptidase | |
| B. thuringiensis | φBtCS33 | PlyBt33 | Amidase | N/A |
| L. monocytogenes | φP40 | PlyP40 | Amidase | N/A |
| L. monocytogenes | φFWLLm3 | LysZ5 | Amidase | MVKYTVENKIIAGLPK |
| | | | | GKLKGANFVIAHETAN |
| | | | | SKSTIDNEVSYMTRN |
| | | | | WQNAFVTHFVGGGG |
| | | | | RVVQVANVNYVSWG |
| | | | | AGQYANSYSYAQVEL |
| | | | | CRTSNATTFKKDYEV |
| | | | | YCQLLVDLAKKAGIPIT |
| | | | | LDSGSKTSDKGIKSHK |
| | | | | WVADKLGGTTHQDPY |
| | | | | AYLSSWGISKAQFAS |
| | | | | DLAKVSGGGNTGTAP |
| | | | | AKPSTPSTNLDKLGLV |
| | | | | DYMNAKKMDSSYSNR |
| | | | | AKLAKQYGIANYSGTA |
| | | | | SQNTTLLSKIKGGAPK |
| | | | | PSTPAPKPSTSTAKKI |
| | | | | YFPPNKGNWSVYPTN |
| | | | | KAPVKANAIGAINPTK |
| | | | | FGGLTYTIQKDRGNG |
| | | | | VYEIQTDQFGRVQVY |
| | | | | GAPSTGAVIKK |
| | | | | (SEQ ID NO: 68) |
| B. cereus | φBPS13 | LysBPS13 | Amidase | MAKREKYIFDVEAEVG |
| | | | | KAAKSIKSLEAELSKL |
| | | | | QKLNKEIDATGGDRTE |
| | | | | KEMLATLKAAKEVNAE |
| | | | | YQKMQRILKDLSKYS |
| | | | | GKVSRKEFNDSKVINN |
| | | | | AKTSVQGGKVTDSFG |
| | | | | QMLKNMERQINSVNK |
| | | | | QFDNHRKAMVDRGQ |
| | | | | QYTPHLKTNRKDSQG |
| | | | | NSNPSMMGRNKSTT |
| | | | | QDMEKAVDKFLNGQN |
| | | | | EATTGLNQALYQLKEI |
| | | | | SKLNRRSESLSRRAS |
| | | | | ASGYMSFQQYSNFTG |
| | | | | DRRTVQQTYGGLKTA |
| | | | | NRERVLELSGQATGIS |
| | | | | KELDRLNSKKGLTARE |
| | | | | GEERKKLMRQLEGID |
| | | | | AELTARKKLNSSLDET |
| | | | | TSNMEKFNQSLVDAQ |
| | | | | VSVKPERGTMRGMM |
| | | | | YERAPAIALAIGGAITA |
| | | | | TIGKLYSEGGNHSKA |
| | | | | MRPDEMYVGQQTGA |
| | | | | VGANWRPNRTATMR |
| | | | | SGLGNHLGFTGQEM |
| | | | | MEFQSNYLSANGYHG |
| | | | | AEDMKAATTGQATFA |
| | | | | RATGLGSDEVKDFFN |
| | | | | TAYRSGGIDGNQTKQ |
| | | | | FQNAFLGAMKQSGAV |
| | | | | GREKDQLKALNGILSS |
| | | | | MSQNRTVSNQDMMR |
| | | | | TVGLQSAISSSGVASL |
| | | | | QGTKGGALMEQLDN |
| | | | | GIREGFNDPQMRVLF |
| | | | | GQGTKYQGMGGRAA |
| | | | | LRKQMEKGISDPDNL |
| | | | | NTLIDASKASAGQDPA |
| | | | | EQAEVLATLASKMGV |
| | | | | NMSSDQARGLIDLQP |
| | | | | SGKLTKENIDKVMKEG |
| | | | | LKEGSIESAKRDKAYS |
| | | | | ESKASIDNSSEAATAK |
| | | | | QATELNDMGSKLRQA |
| | | | | NAALGGLPAPLYTAIA |
| | | | | AVVAFTAAVAGSALM |
| | | | | FKGASWLKGGMASKY |
| | | | | GGKGGKGGKGGGTG |
| | | | | GGGGAGGAAATGAG |
| | | | | AAAGAGGVGAAAAGE |
| | | | | VGAGVAAGGAAAGAA |
| | | | | AGGSKLAGVGKGFMK |
| | | | | GAGKLMLPLGILMGAS |
| | | | | EIMQAPEEAKGSAIGS |
| | | | | AVGGIGGGIAGGAAT |
| | | | | GAIAGSFLGPIGTAVG |
| | | | | GIAGGIAGGFAGSSLG |
| | | | | ETIGGWFDSGPKEDA |
| | | | | SAADKAKADASAAAL |
| | | | | AAAAGTSGAVGSSAL |
| | | | | QSQMAQGITGAPNMS |
| | | | | QVGSMASALGISSGA |
| | | | | MASALGISSGQENQIQ |
| | | | | TMTDKENTNTKKANE |
| | | | | AKKGDNLSYERENIS |
| | | | | MYERVLTRAEQILAQA |
| | | | | RAQNGIMGVGGGGTA |
| | | | | GAGGGINGFTGGGKL |
| | | | | QFLAAGQKWSSSNLQ |
| | | | | QHDLGFTDQNLTAED |
| | | | | LDKWIDSKAPQGSMM |
| | | | | RGMGATFLKAGQEYG |
| | | | | LDPRYLIAHAAEESGW |
| | | | | GTSKIARDKGNFFGIG |
| | | | | AFDDSPYSSAYEFKD |
| | | | | GTGSAAERGIMGGAK |
| | | | | WISEKYYGKGNTTLD |
| | | | | KMKAAGYATNASWAP |
| | | | | NIASIMAGAPTGSGSG |
| | | | | NVTATINVNVKGDEKV |
| | | | | SDKLKNSSDMKKAGK |
| | | | | DIGSLLGFYSREMTIA |
| | | | | (SEQ ID NO: 69) |
| S. aureus | φGH15 | LysGH15 | Amidase and | MAKTQAEINKRLDAYA |
| | | | endopeptidase | KGTVDSPYRIKKATSY |
| | | | | DPSFGVMEAGAIDAD |
| | | | | GYYHAQCQDLITDYVL |
| | | | | WLTDNKVRTWGNAK |
| | | | | DQIKQSYGTGFKIHEN |
| | | | | KPSTVPKKGWIAVFTS |
| | | | | GSYQQWGHIGIVYDG |
| | | | | GNTSTFTILEQNWNG |
| | | | | YANKKPTKRVDNYYG |
| | | | | LTHFIEIPVKAGTTVKK |
| | | | | ETAKKSASKTPAPKKK |
| | | | | ATLKVSKNHINYTMDK |
| | | | | RGKKPEGMVIHNDAG |
| | | | | RSSGQQYENSLANAG |
| | | | | YARYANGIAHYYGSE |
| | | | | GYVWEAIDAKNQIAW |
| | | | | HTGDGTGANSGNFRF |
| | | | | AGIEVCQSMSASDAQ |
| | | | | FLKNEQAVFQFTAEKF |
| | | | | KEWGLTPNRKTVRLH |
| | | | | MEFVPTACPHRSMVL |
| | | | | HTGFNPVTQGRPSQA |
| | | | | IMNKLKDYFIKQIKNYM |
| | | | | DKGTSSSTVVKDGKT |
| | | | | SSASTPATRPVTGSW |
| | | | | KKNQYGTWYKPENAT |
| | | | | FVNGNQPIVTRIGSPF |
| | | | | LNAPVGGNLPAGATIV |
| | | | | YDEVCIQAGHIWIGYN |
| | | | | AYNGDRVYCPVRTCQ |
| | | | | GVPPNHIPGVAWGVFK |
| | | | | (SEQ ID NO: 70) |
| S. aureus | φvB SauS- | HydH5 | Endopeptidase | N/A |
| | PLA88 | | and glycosidase | |
| E. faecalis | φF168/08 | Lys168 | Endopeptidase | N/A |
| E. faecalis | φF170/08 | Lys170 | Amidase | N/A |
| S. aureus | φP-27/HP | P-27/HP | Nonspecified | N/A |
| C. perfringens | φSM101 | Psm | Muramidase | N/A |
| C. sporogenes | φ8074-B1 | CS74L | Amidase | MKIGIDMGHTLSGADY |
| | | | | GVVGLRPESVLTREV |
| | | | | GTKVIYKLQKLGHVVV |
| | | | | NCTVDKASSVSESLYT |
| | | | | RYYRANQANVDLFISI |
| | | | | HFNATPGGTGTEVYT |
| | | | | YAGRQLGEATRIRQE |
| | | | | FKSLGLRDRGTKDGS |
| | | | | GLAVIRNTKAKAMLVE |
| | | | | CCFCDNPNDMKLYNS |
| | | | | ESFSNAIVKGITGKLP |
| | | | | NGESGNNNQGGNKV |
| | | | | KAVVIYNEGADRRGA |
| | | | | EYLADYLNCPTISNSR |
| | | | | TFDYSCVEHVYAVGG |
| | | | | KKEQYTKYLKTLLSGA |
| | | | | NRYDTMQQILNFINGGK |
| | | | | (SEQ ID NO: 71) |
| S. typhimurium | φSPN1S | SPN1S | Glycosidase | MDINQFRRASGINEQL |
| | | | | AARWFPHITTAMNEF |
| | | | | GITKPDDQAMFIAQVG |
| | | | | HESGGFTRLQENFNY |
| | | | | SVNGLSGFIRAGRITP |
| | | | | DQANALGRKTYEKSL |
| | | | | PLERQRAIANLVYSKR |
| | | | | MGNNGPGDGWNYRG |
| | | | | RGLIQITGLNNYRDCG |
| | | | | NGLKVDLVAQPELLA |
| | | | | QDEYAARSAAWFFSS |
| | | | | KGCMKYTGDLVRVTQ |
| | | | | IINGGQNGIDDRRTRY |
| | | | | AAARKVLAL |
| | | | | (SEQ ID NO: 72) |
| C. michiganensis | φCMP1 | CMP1 | Peptidase | N/A |
| C. michiganensis | φCN77 | CN77 | Peptidase | MGYWGYPNGQIPND |
| | | | | KMALYRGCLLRADAA |
| | | | | AQAYALQDAYTRATG |
| | | | | KPLVILEGYRDLTRQK |
| | | | | YLRNLYLSGRGNIAAV |
| | | | | PGLSNHGWGLACDFA |
| | | | | APLNSSGSEEHRWM |
| | | | | RQNAPLFGFDWARGK |
| | | | | ADNEPWHWEYGNVP |
| | | | | VSRWASLDVTPIDRN |
| | | | | DMADITEGQMQRIAVI |
| | | | | LLDTEIQTPLGPRLVK |
| | | | | HALGDALLLGQANAN |
| | | | | SIAEVPDKTWDVLVDH |
| | | | | PLAKNEDGTPLKVRL |
| | | | | GDVAKYEPLEHQNTR |
| | | | | DAIAKLGTLQFTDKQL |
| | | | | ATIGAGVKPIDEASLV |
| | | | | KKIVDGVRALFGRAAA |
| | | | | (SEQ ID NO: 73) |
| A. baumannii | φAB2 | LysAB2 | Glycosidase | MILTKDGFSIIRNELFG |
| | | | | GKLDQTQVDAINFIVA |
| | | | | KATESGLTYPEAAYLL |
| | | | | ATIYHETGLPSGYRTM |
| | | | | QPIKEAGSDSYLRSKK |
| | | | | YYPYIGYGYVQLTWK |
| | | | | ENYERIGKLIGVDLIKN |
| | | | | PEKALEPLIAIQIAIKGM |
| | | | | LNGWFTGVGFRRKRP |
| | | | | VSKYNKQQYVAARNII |
| | | | | NGKDKAELIAKYAIIFE |
| | | | | RALRSL |
| | | | | (SEQ ID NO: 74) |
| B. cereus | φB4 | LysB4 | Endopeptidase | MAMALQTLIDKANRKL |
| | | | | NVSGMRKDVADRTRA |
| | | | | VITQMHAQGIYICVAQ |
| | | | | GFRSFAEQNALYAQG |
| | | | | RTKPGSIVTNARGGQ |
| | | | | SNHNYGVAVDLCLYT |
| | | | | QDGSDVIWTVEGNFR |
| | | | | KVIAAMKAQGFKWGG |
| | | | | DWVSFKDYPHFELYD |
| | | | | VVGGQKPPADNGGA |
| | | | | VDNGGGSGSTGGSG |
| | | | | GGSTGGGSTGGGYD |
| | | | | SSWFTKETGTFVTNT |
| | | | | SIKLRTAPFTSADVIAT |
| | | | | LPAGSPVNYNGFGIEY |
| | | | | DGYVWIRQPRSNGYG |
| | | | | YLATGESKGGKRQNY |
| | | | | WGTFK |
| | | | | (SEQ ID NO: 75) |
| P. aeruginosa | φKMV | KMV45 | Nonspecified | N/A |
| C. tyrobutyricum | φCTP1 | Ctp1l | Glycosidase | MKKIADISNLNGNVDV |
| | | | | KLLFNLGYIGIIAKASE |
| | | | | GGTFVDKYYKQNYTN |
| | | | | TKAQGKITGAYHFANF |
| | | | | STIAKAQQEANFFLNC |
| | | | | IAGTTPDFVVLDLEQQ |
| | | | | CTGDITDACLAFLNIVA |
| | | | | KKFKCVVYCNSSFIKE |
| | | | | HLNSKICAYPLWIANY |
| | | | | GVATPAFTLWTKYAM |
| | | | | WQFTEKGQVSGISGYI |
| | | | | DFSYITDEFIKYIKGED |
| | | | | EVENLVVYNDGADQR |
| | | | | AAEYLADRLACPTINN |
| | | | | ARKFDYSNVKNVYAV |
| | | | | GGNKEQYTSYLTTLIA |
| | | | | GSTRYTTMQAVLDYIK |
| | | | | NLK |
| | | | | (SEQ ID NO: 76) |
| P. aeruginosa | φEL | EL188 | Transglycosylase | N/A |
| P. aeruginosa | φKZ | KZ144 | Transglycosylase | N/A |
| S. aureus | Staphylococcus | Ply187 | Cell Wall | MALPKTGKPTAKQVV |
| | virus 187 | | Hydrolase | DWAINLIGSGVDVDGY |
| | | | | YGRQCWDLPNYIFNR |
| | | | | YVVNFKTPGNARDMA |
| | | | | WYRYPEGFKVFRNTS |
| | | | | DFVPKPGDIAVWTGG |
| | | | | NYNWNTVVGHTGIVVG |
| | | | | PSTKSYFYSVDQNWN |
| | | | | NSNSYVGSPAAKIKHS |
| | | | | YFGVTHFVRPAYKAE |
| | | | | PKPTPPAQNNPAPKD |
| | | | | PEPSKKPESNKPIYKV |
| | | | | VTKILFTTAHIEHVKAN |
| | | | | RFVHYITKSDNHNNKP |
| | | | | NKIVIKNTNTALSTIDV |
| | | | | YRYRDELDKDEIPHFF |
| | | | | VDRLNVWACRPIEDSI |
| | | | | NGYHDSVVLSITETRT |
| | | | | ALSDNFKMNEIECLSL |
| | | | | AESILKANNKKMSASN |
| | | | | IIVDNKAWRTFKLHTG |
| | | | | KDSLKSSSFTSKDYQ |
| | | | | KAVNELIKLFNDKDKL |
| | | | | LNNKPKDVVERIRIRTI |
| | | | | VKENTKFVPSELKPRN |
| | | | | NIRDKQDSKIDRVINN |
| | | | | YTLKQALNIQYKLNPK |
| | | | | PQTSNGVSWYNASVN |
| | | | | QIKSAMDTTKIFNNNV |
| | | | | QVYQFLKLNQYQGIPV |
| | | | | DKLNKLLVGKGTLAN |
| | | | | QGHAFADGCKKYNIN |
| | | | | EIYLIAHRFLESANGTS |
| | | | | FFASGKTGVYNYFGIG |
| | | | | AFDNNPNNAMAFARS |
| | | | | HGWTSPTKAIIGGAEF |
| | | | | VGKGYFNVGQNTLYR |
| | | | | MRWNPQKPGTHQYA |
| | | | | TDISWAKVQAQMISA |
| | | | | MYKEIGLTGDYFIYDQ |
| | | | | YKK |
| | | | | (SEQ ID NO: 77) |
| P. uorescens | φOBP | OBPgp279 | Glycosidase | N/A |
| L. monocytogenes | φP35 | PlyP35 | Amidase | MARKFTKAELVAKAE |
| | | | | KKVGGLKPDVKKAVL |
| | | | | SAVKEAYDRYGIGIIVS |
| | | | | QGYRSIAEQNGLYAQ |
| | | | | GRTKPGNIVTNAKGG |
| | | | | QSNHNFGVAVDFAIDL |
| | | | | IDDGKIDSWQPSATIV |
| | | | | NMMKRRGFKWGGD |
| | | | | WKSFTDLPHFEACDW |
| | | | | YRGERKYKVDTSEWK |
| | | | | KKENINIVIKDVGYFQD |
| | | | | KPQFLNSKSVRQWKH |
| | | | | GTKVKLTKHNSHWYT |
| | | | | GVVKDGNKSVRGYIY |
| | | | | HSMAKVTSKNSDGSV |
| | | | | NATINAHAFCWDNKK |
| | | | | LNGGDFINLKRGFKGI |
| | | | | THPASDGFYPLYFAS |
| | | | | RKKTFYIPRYMFDIKK |
| | | | | (SEQ ID NO: 78) |
| L. fermentum | φPYB5 | Lyb5 | Muramidase | N/A |
| S. pneumoniae | φCP-7 | Cpl-7 | Muramidase | MVKKNDLFVDVASHQ |
| | | | | GYDISGILEEAGTTNTII |
| | | | | KVSESTSYLNPCLSAQ |
| | | | | VSQSNPIGFYHFAWF |
| | | | | GGNEEEAEAEARYFL |
| | | | | DNVPTQVKYLVLDYE |
| | | | | DHASASVQRNTTACL |
| | | | | RFMQIIAEAGYTPIYYS |
| | | | | YKPFTLDNVDYQQILA |
| | | | | QFPNSLWIAGYGLND |
| | | | | GTANFEYFPSMDGIR |
| | | | | WWQYSSNPFDKNIVL |
| | | | | LDDEKEDNINNENTLK |
| | | | | SLTTVANEVIQGLWG |
| | | | | NGQERYDSLANAGYD |
| | | | | PQAVQDKVNEILNARE |
| | | | | IADLTTVANEVIQGLW |
| | | | | GNGQERYDSLANAGY |
| | | | | DPQAVQDKVNEILNA |
| | | | | REIADLTTVANEVIQGL |
| | | | | WGNGQERYDSLANA |
| | | | | GYDPQAVQDKVNELLS |
| | | | | (SEQ ID NO: 79) |
| P. chlororaphis201 | φ2-1 | 201φ2- | Glycosidase | N/A |
| | | 1gp229 | | |
| S. enterica | φPVP-SE1) | PVP- | Glycosidase | N/A |
| | | SE1gp146 | | |
| Corynebacterium | φBFK20 | BKF20 | Amidase | N/A |
| E. faecalis | φEFAP-1 | EFAL-1 | Amidase | MKLKGILLSVVTTFGLL |
| | | | | FGATNVQAYEVNNEF |
| | | | | NLQPWEGSQQLAYPN |
| | | | | KIILHETANPRATGRN |
| | | | | EATYMKNNWFNAHTT |
| | | | | AIVGDGGIVYKVAPEG |
| | | | | NVSWGAGNANPYAP |
| | | | | VQIELQHTNDPELFKA |
| | | | | NYKAYVDYTRDMGKK |
| | | | | FGIPMTLDQGGSLWE |
| | | | | KGVVSHQWVTDFVW |
| | | | | GDHTDPYGYLAKMGI |
| | | | | SKAQLAHDLANGVSG |
| | | | | NTATPTPKPDKPKPT |
| | | | | QPSKPSNKKRFNYRV |
| | | | | DGLEYVNGMWQIYNE |
| | | | | HLGKIDFNWTENGIPV |
| | | | | EVVDKVNPATGQPTK |
| | | | | DQVLKVGDYFNFQEN |
| | | | | STGVVQEQTPYMGYT |
| | | | | LSHVQLPNEFIWLFTD |
| | | | | SKQALMYQ |
| | | | | (SEQ ID NO: 80) |
| Lactobacilli | lambdaSA2 | LysA, | Nonspecified | N/A |
| | | LysA2, and | | |
| | | Lysga Y | | |
| S. aureus | | SAL-1 | Nonspecified | N/A |

In some instances, the lysin is a functionally active variant of the lysins described herein. In some instances, the variant of the lysin has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a lysin described herein or a naturally occurring lysin.

In some instances, the lysin may be bioengineered to modulate its bioactivity, e.g., increase or decrease or regulate, or to specify a target microorganism. In some instances, the lysin is produced by the translational machinery (e.g. a ribosome, etc.) of a microbial cell. In some instances, the lysin is chemically synthesized. In some instances, the lysin is derived from a polypeptide precursor. The polypeptide precursor can undergo cleavage (for example, processing by a protease) to yield the polypeptide of the lysin itself. As such, in some instances, the lysin is produced from a precursor polypeptide. In some instances, the lysin includes a polypeptide that has undergone post-translational modifications, for example, cleavage, or the addition of one or more functional groups.

The lysins described herein may be formulated in a composition for any of the uses described herein. The compositions disclosed herein may include any number or type (e.g., classes) of lysins, such as at least about any one of 1 lysin, 2, 3, 4, 5, 10, 15, 20, or more lysins. A suitable concentration of each lysin in the composition depends on factors such as efficacy, stability of the lysin, number of distinct lysin, the formulation, and methods of application of the composition. In some instances, each lysin in a liquid composition is from about 0.1 ng/mL to about 100 mg/mL. In some instances, each lysin in a solid composition is from about 0.1 ng/g to about 100 mg/g. In some instances, wherein the composition includes at least two types of lysins, the concentration of each type of lysin may be the same or different.

A modulating agent including a lysin as described herein can be contacted with the target host in an amount and for a time sufficient to: (a) reach a target level (e.g., a predetermined or threshold level) of lysin concentration inside a target host; (b) reach a target level (e.g., a predetermined or threshold level) of lysin concentration inside a target host gut; (c) reach a target level (e.g., a predetermined or threshold level) of lysin concentration inside a target host bacteriocyte; (d) modulate the level, or an activity, of one or more microorganism (e.g., endosymbiont) in the target host; or/and (e) modulate fitness of the target host.

(c) Antimicrobial Peptides

The modulating agent described herein may include an antimicrobial peptide (AMP). Any AMP suitable for inhibiting a microorganism resident in the host may be used. AMPs are a diverse group of molecules, which are divided into subgroups on the basis of their amino acid composition and structure. The AMP may be derived or produced from any organism that naturally produces AMPs, including AMPs derived from plants (e.g., copsin), insects (e.g., drosocin, scorpion peptide (e.g., Uy192, UyCT3, D3, D10, Uy17, Uy192), mastoparan, poneratoxin, cecropin, moricin, melittin), frogs (e.g., magainin, dermaseptin, aurein), and mammals (e.g., cathelicidins, defensins and protegrins). In some instances, the AMP may be a scorpion peptide, such as Uy192 (5′-FLSTIWNGIKGLL-3′; SEQ ID NO: 232), UyCT3 (5′-LSAIWSGIKSLF-3; SEQ ID NO: 233), D3 (5′-LWGKLWEGVKSLI-3′; SEQ ID NO: 234), and D10 (5′-FPFLKLSLKIPKSAIKSAIKRL-3′; SEQ ID NO: 235), Uy17 (5′-ILSAIWSGIKGLL-3′; SEQ ID NO: 236), or a combination thereof. In some instances, the antimicrobial peptide may be one having at least 90% sequence identity (e.g., at least 90%, 92%, 94%, 96%, 98%, or 100% sequence identity) with one or more of the following: cecropin (SEQ ID NO: 82), melittin, copsin, drosomycin (SEQ ID NO: 93), dermcidin (SEQ ID NO: 81), andropin (SEQ ID NO: 83), moricin (SEQ ID NO: 84), ceratotoxin (SEQ ID NO: 85), abaecin (SEQ ID NO: 86), apidaecin (SEQ ID NO: 87), prophenin (SEQ ID NO: 88), indolicidin (SEQ ID NO: 89), protegrin (SEQ ID NO: 90), tachyplesin (SEQ ID NO: 91), or defensin (SEQ ID NO: 92) to the agricultural insect pest. Non-limiting examples of AMPs are listed in Table 6.

TABLE 6
Examples of Antimicrobial Peptides

| | | Example | |
|---|---|---|---|
| Type | Characteristic | AMP | Sequence |
| Anionic | rich in glutamic and | dermcidin | SSLLEKGLDGAKKAVGGLGKL |
| peptides | aspartic acid | | GKDAVEDLESVGKGAVHDVKD |
| | | | VLDSVL |
| | | | (SEQ ID NO: 81) |
| Linear cationic | lack cysteine | cecropin A | KWKLFKKIEKVGQNIRDGIIKAG |
| α-helical | | | PAVAVVGQATQIAK |
| peptides | | | (SEQ ID NO: 82) |
| | | andropin | MKYFSVLVVLTLILAIVDQSDAFI |
| | | | NLLDKVEDALHTGAQAGFKLIR |
| | | | PVERGATPKKSEKPEK |
| | | | (SEQ ID NO: 83) |
| | | moricin | MNILKFFFVFIVAMSLVSCSTAA |
| | | | PAKIPIKAIKTVGKAVGKGLRAI |
| | | | NIASTANDVFNFLKPKKRKH |
| | | | (SEQ ID NO: 84) |
| | | ceratotoxin | MANLKAVFLICIVAFIALQCVVA |
| | | | EPAAEDSVVVKRSIGSALKKAL |
| | | | PVAKKIGKIALPIAKAALPVAAG |
| | | | LVG |
| | | | (SEQ ID NO: 85) |
| Cationic | rich in proline, arginine, | abaecin | MKVVIFIFALLATICAAFAYVPLP |
| peptide | phenylalanine, glycine, | | NVPQPGRRPFPTFPGQGPFNP |
| enriched for | tryptophan | | KIKWPQGY |
| specific amino | | | (SEQ ID NO: 86) |
| acid | | | |
| | | apidaecins | KNFALAILVVTFVVAVFGNTNLD |
| | | | PPTRPTRLRREAKPEAEPGNN |
| | | | RPVYIPQPRPPHPRLRREAEPE |
| | | | AEPGNNRPVYIPQPRPPHPRL |
| | | | RREAELEAEPGNNRPVYISQP |
| | | | RPPHPRLRREAEPEAEPGNNR |
| | | | PVYIPQPRPPHPRLRREAELEA |
| | | | EPGNNRPVYISQPRPPHPRLR |
| | | | REAEPEAEPGNNRPVYIPQPR |
| | | | PPHPRLRREAEPEAEPGNNRP |
| | | | VYIPQPRPPHPRLRREAEPEAE |
| | | | PGNNRPVYIPQPRPPHPRLRR |
| | | | EAKPEAKPGNNRPVYIPQPRP |
| | | | PHPRI |
| | | | (SEQ ID NO: 87) |
| | | prophenin | METQRASLCLGRWSLWLLLLA |
| | | | LVVPSASAQALSYREAVLRAVD |
| | | | RLNEQSSEANLYRLLELDQPPK |
| | | | ADEDPGTPKPVSFTVKETVCP |
| | | | RPTRRPPELCDFKENGRVKQC |
| | | | VGTVTLDQIKDPLDITCNEGVR |
| | | | RFPWWWPFLRRPRLRRQAFP |
| | | | PPNVPGPRFPPPNVPGPRFPP |
| | | | PNFPGPRFPPPNFPGPRFPPP |
| | | | NFPGPPFPPPIFPGPWFPPPPP |
| | | | FRPPPFGPPRFPGRR |
| | | | (SEQ ID NO: 88) |
| | | indolicidin | MQTQRASLSLGRWSLWLLLLG |
| | | | LVVPSASAQALSYREAVLRAVD |
| | | | QLNELSSEANLYRLLELDPPPK |
| | | | DNEDLGTRKPVSFTVKETVCP |
| | | | RTIQQPAEQCDFKEKGRVKQC |
| | | | VGTVTLDPSNDQFDLNCNELQ |
| | | | SVILPWKWPWWPWRRG |
| | | | (SEQ ID NO: 89) |
| Anionic and | contain 1-3 disulfide bond | protegrin | METQRASLCLGRWSLWLLLLA |
| cationic | | | LVVPSASAQALSYREAVLRAVD |
| peptides that | | | RLNEQSSEANLYRLLELDQPPK |
| contain | | | ADEDPGTPKPVSFTVKETVCP |
| cysteine and | | | RPTRQPPELCDFKENGRVKQC |
| form disulfide | | | VGTVTLDQIKDPLDITCNEVQG |
| bonds | | | VRGGRLCYCRRRFCVCVGRG |
| | | | (SEQ ID NO: 90) |
| | | tachyplesins | KWCFRVCYRGICYRRCR |
| | | | (SEQ ID NO: 91) |
| | | defensin | MKCATIVCTIAVVLAATLLNGSV |
| | | | QAAPQEEAALSGGANLNTLLD |
| | | | ELPEETHHAALENYRAKRATC |
| | | | DLASGFGVGSSLCAAHCIARR |
| | | | YRGGYCNSKAVCVCRN |
| | | | (SEQ ID NO: 92) |
| | | drosomycin | MMQIKYLFALFAVLMLVVLGAN |
| | | | EADADCLSGRYKGPCAVWDN |
| | | | ETCRRVCKEEGRSSGHCSPSL |
| | | | KCWCEGC |
| | | | (SEQ ID NO: 93) |

The AMP may be active against any number of target microorganisms. In some instances, the AMP may have antibacterial and/or antifungal activities. In some instances, the AMP may have a narrow-spectrum bioactivity or a broad-spectrum bioactivity. For example, some AMPs target and kill only a few species of bacteria or fungi, while others are active against both gram-negative and gram-positive bacteria as well as fungi.

Further, the AMP may function through a number of known mechanisms of action. For example, the cytoplasmic membrane is a frequent target of AMPs, but AMPs may also interfere with DNA and protein synthesis, protein folding, and cell wall synthesis. In some instances, AMPs with net cationic charge and amphipathic nature disrupt bacterial membranes leading to cell lysis. In some instances, AMPs may enter cells and interact with intracellular target to interfere with DNA, RNA, protein, or cell wall synthesis. In addition to killing microorganisms, AMPs have demonstrated a number of immunomodulatory functions that are involved in the clearance of infection, including the ability to alter host gene expression, act as chemokines and/or induce chemokine production, inhibit lipopolysaccharide induced pro-inflammatory cytokine production, promote wound healing, and modulating the responses of dendritic cells and cells of the adaptive immune response.

In some instances, the AMP is a functionally active variant of the AMPs described herein. In some instances, the variant of the AMP has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of an AMP described herein or a naturally derived AMP.

In some instances, the AMP may be bioengineered to modulate its bioactivity, e.g., increase or decrease or regulate, or to specify a target microorganism. In some instances, the AMP is produced by the translational machinery (e.g. a ribosome, etc.) of a cell. In some instances, the AMP is chemically synthesized. In some instances, the AMP is derived from a polypeptide precursor. The polypeptide precursor can undergo cleavage (for example, processing by a protease) to yield the polypeptide of the AMP itself. As such, in some instances, the AMP is produced from a precursor polypeptide. In some instances, the AMP includes a polypeptide that has undergone post-translational modifications, for example, cleavage, or the addition of one or more functional groups.

The AMPs described herein may be formulated in a composition for any of the uses described herein. The compositions disclosed herein may include any number or type (e.g., classes) of AMPs, such as at least about any one of 1 AMP, 2, 3, 4, 5, 10, 15, 20, or more AMPs. For example, the compositions may include a cocktail of AMPs (e.g., a cocktail of scorpion peptides, e.g., UyCT3, D3, D10, and Uy17). A suitable concentration of each AMP in the composition depends on factors such as efficacy, stability of the AMP, number of distinct AMP in the composition, the formulation, and methods of application of the composition. In some instances, each AMP in a liquid composition is from about 0.1 ng/mL to about 100 mg/mL (about 0.1 ng/mL to about 1 ng/mL, about 1 ng/mL to about 10 ng/mL, about 10 ng/mL to about 100 ng/mL, about 100 ng/mL to about 1000 ng/mL, about 1 mg/mL to about 10 mg/mL, about 10 mg/mL to about 100 mg/mL). In some instances, each AMP in a solid composition is from about 0.1 ng/g to about 100 mg/g (about 0.1 ng/g to about 1 ng/g, about 1 ng/g to about 10 ng/g, about 10 ng/g to about 100 ng/g, about 100 ng/g to about 1000 ng/g, about 1 mg/g to about 10 mg/g, about 10 mg/g to about 100 mg/g). In some instances, wherein the composition includes at least two types of AMPs, the concentration of each type of AMP may be the same or different.

A modulating agent including an AMP as described herein can be contacted with the target host in an amount and for a time sufficient to: (a) reach a target level (e.g., a predetermined or threshold level) of AMP concentration inside a target host; (b) reach a target level (e.g., a predetermined or threshold level) of AMP concentration inside a target host gut; (c) reach a target level (e.g., a predetermined or threshold level) of AMP concentration inside a target host bacteriocyte; (d) modulate the level, or an activity, of one or more microorganism (e.g., endosymbiont) in the target host; or/and (e) modulate fitness of the target host.

As illustrated by Examples 17-19, AMPs, such as scorpion peptides, can be used as modulating agents that target an endosymbiotic bacterium, such as a Buchnera spp., in an insect host, such as aphids, to decrease the fitness of the host (e.g., as outlined herein).

(d) Nodule C-Rich Peptides

The modulating agent described herein may include a nodule C-rich peptide (NCR peptide). NCR peptides are produced in certain leguminous plants and play an important role in the mutualistic, nitrogen-fixing symbiosis of the plants with bacteria from the Rhizobiaceae family (rhizobia), resulting in the formation of root nodules where plant cells contain thousands of intracellular endosymbionts. NCR peptides possess anti-microbial properties that direct an irreversible, terminal differentiation process of bacteria, e.g., to permeabilize the bacterial membrane, disrupt cell division, or inhibit protein synthesis. For example, in Medicago truncatula nodule cells infected with Sinorhizobium meliloti, hundreds of NCR peptides are produced which direct irreversible differentiation of the bacteria into large polyploid nitrogen-fixing bacteroids.). Non-limiting examples of NCR peptides are listed in Table 7.

TABLE 7
Examples of NCR Peptides

| NAME | Peptide sequence | Producer |
|---|---|---|
| >gi\|152218086\|gb\|ABS31477.1\| | MTKIVVFIYVVILLLTIFHVSAKKKRYI | Medicago truncatula |
| NCR 340 | ECETHEDCSQVFMPPFVMRCVIHE | |
| | CKIFNGEHLRY | |
| | (SEQ ID NO: 94) | |
| >gi\|152218084\|gb\|ABS31476.1\| | MAKIMKFVYNMIPFLSIFIITLQVNVV | Medicago truncatula |
| NCR 339 | VCEIDADCPQICMPPYEVRCVNHRC | |
| | GWVNTDDSLFLTQEFTRSKQYIIS | |
| | (SEQ ID NO: 95) | |
| >gi\|152218082\|gb\|ABS31475.1\| | MYKVVESIFIRYMHRKPNMTKFFKF | Medicago truncatula |
| NCR 338 | VYTMFILISLFLVVTNANAHNCTDISD | |
| | CSSNHCSYEGVSLCMNGQCICIYE | |
| | (SEQ ID NO: 96) | |
| >gi\|152218080\|gb\|ABS31474.1\| | MVETLRLFYIMILFVSLCLVVVDGES | Medicago truncatula |
| NCR 337 | KLEQTCSEDFECYIKNPHVPFGHLR | |
| | CFEGFCQQLNGPA | |
| | (SEQ ID NO: 97) | |
| >gi\|152218078\|gb\|ABS31473.1\| | MAKIVNFVYSMIVFLFLFLVATKAAR | Medicago truncatula |
| NCR 336 | GYLCVTDSHCPPHMCPPGMEPRCV | |
| | RRMCKCLPIGWRKYFVP | |
| | (SEQ ID NO: 98) | |
| >gi\|152218076\|gb\|ABS31472.1\| | MQIGKNMVETPKLDYVIIFFFLYFFF | Medicago truncatula |
| NCR 335 | RQMIILRLNTTFRPLNFKMLRFWGQ | |
| | NRNIMKHRGQKVHFSLILSDCKTNK | |
| | DCPKLRRANVRCRKSYCVPI | |
| | (SEQ ID NO: 99) | |
| >gi\|152218074\|gb\|ABS31471.1\| | MLRLYLVSYFLLKRTLLVSYFSYFST | Medicago truncatula |
| NCR 334 | YIIECKTDNDCPISQLKIYAWKCVKN | |
| | GCHLFDVIPMMYE | |
| | (SEQ ID NO: 100) | |
| >gi\|152218072\|gb\|ABS31470.1\| | MAEILKFVYIVILFVSLLLIVVASEREC | Medicago truncatula |
| NCR 333 | VTDDDCEKLYPTNEYRMMCDSGYC | |
| | MNLLNGKIIYLLCLKKKKFLIIISVLL | |
| | (SEQ ID NO: 101) | |
| >gi\|152218070\|gb\|ABS31469.1\| | MAEIIKFVYIMILCVSLLLIEVAGEECV | Medicago truncatula |
| NCR 332 | TDADCDKLYPDIRKPLMCSIGECYSL | |
| | YKGKFSLSIISKTSFSLMVYNVVTLVI | |
| | CLRLAYISLLLKFL | |
| | (SEQ ID NO: 102) | |
| >gi\|152218068\|gb\|ABS31468.1\| | MAEILKDFYAMNLFIFLIILPAKIRGET | Medicago truncatula |
| NCR 331 | LSLTHPKCHHIMLPSLFITEVFQRVT | |
| | DDGCPKPVNHLRVVKCIEHICEYGY | |
| | NYRPDFASQIPESTKMPRKRE | |
| | (SEQ ID NO: 103) | |
| >gi\|152218066\|gb\|ABS31467.1\| | MVEILKNFYAMNLFIFLIILAVKIRGAH | Medicago truncatula |
| NCR 330 | FPCVTDDDCPKPVNKLRVIKCIDHIC | |
| | QYARNLPDFASEISESTKMPCKGE | |
| | (SEQ ID NO: 104) | |
| >gi\|152218064\|gb\|ABS31466.1\| | MFHAQAENMAKVSNFVCIMILFLALF | Medicago truncatula |
| NCR 329 | FITMNDAARFECREDSHCVTRIKCV | |
| | LPRKPECRNYACGCYDSNKYR | |
| | (SEQ ID NO: 105) | |
| >gi\|152218062\|gb\|ABS31465.1\| | MQMRQNMATILNFVFVIILFISLLLVV | Medicago truncatula |
| NCR 328 | TKGYREPFSSFTEGPTCKEDIDCPSI | |
| | SCVNPQVPKCIMFECHCKYIPTTLK | |
| | (SEQ ID NO: 106) | |
| >gi\|152218060\|gb\|ABS31464.1\| | MATILMYVYITILFISILTVLTEGLYEPL | Medicago truncatula |
| NCR 327 | YNFRRDPDCRRNIDCPSYLCVAPKV | |
| | PRCIMFECHCKDIPSDH | |
| | (SEQ ID NO: 107) | |
| >gi\|152218058\|gb\|ABS31463.1\| | MTTSLKFVYVAILFLSLLLVVMGGIR | Medicago truncatula |
| NCR 326 | RFECRQDSDCPSYFCEKLTVPKCF | |
| | WSKCYCK | |
| | (SEQ ID NO: 108) | |
| >gi\|152218056\|gb\|ABS31462.1\| | MTTSLKFVYVAILFLSLLLVVMGGIR | Medicago truncatula |
| NCR 325 | KKECRQDSDCPSYFCEKLTIAKCIHS | |
| | TCLCK | |
| | (SEQ ID NO: 109) | |
| >gi\|152218054\|gb\|ABS31461.1\| | MQIGKNMVETPKLVYFIILFLSIFLCIT | Medicago truncatula |
| NCR 324 | VSNSSFSQIFNSACKTDKDCPKFGR | |
| | VNVRCRKGNCVPI | |
| | (SEQ ID NO: 110) | |
| >gi\|152218046\|gb\|ABS31457.1\| | MTAILKKFINAVFLFIVLFLATTNVED | Medicago truncatula |
| NCR 320 | FVGGSNDECVYPDVFQCINNICKCV | |
| | SHHRT | |
| | (SEQ ID NO: 111) | |
| >gi\|152218044\|gb\|ABS31456.1\| | MQKRKNMAQIIFYVYALIILFSPFLAA | Medicago truncatula |
| NCR 319 | RLVFVNPEKPCVTDADCDRYRHES | |
| | AIYSDMFCKDGYCFIDYHHDPYP | |
| | (SEQ ID NO: 112) | |
| >gi\|152218042\|gb\|ABS31455.1\| | MQMRKNMAQILFYVYALLILFTPFLV | Medicago truncatula |
| NCR 318 | ARIMVVNPNNPCVTDADCQRYRHK | |
| | LATRMICNQGFCLMDFTHDPYAPSL | |
| | P | |
| | (SEQ ID NO: 113) | |
| >gi\|152218040\|gb\|ABS31454.1\| | MNHISKFVYALIIFLSIYLVVLDGLPIS | Medicago truncatula |
| NCR 317 | CKDHFECRRKINILRCIYRQEKPMCI | |
| | NSICTCVKLL | |
| | (SEQ ID NO: 114) | |
| >gi\|152218038\|gb\|ABS31453.1\| | MQREKNMAKIFEFVYAMIIFILLFLVE | Medicago truncatula |
| NCR 316 | KNVVAYLKFECKTDDDCQKSLLKTY | |
| | VWKCVKNECYFFAKK | |
| | (SEQ ID NO: 115) | |
| >gi\|152218036\|gb\|ABS31452.1\| | MAGIIKFVHVLIIFLSLFHVVKNDDGS | Medicago truncatula |
| NCR 315 | FCFKDSDCPDEMCPSPLKEMCYFL | |
| | QCKCGVDTIA | |
| | (SEQ ID NO: 116) | |
| >gi\|152218034\|gb\|ABS31451.1\| | MANTHKLVSMILFIFLFLASNNVEGY | Medicago truncatula |
| NCR 314 | VNCETDADCPPSTRVKRFKCVKGE | |
| | CRWTRMSYA | |
| | (SEQ ID NO: 117) | |
| >gi\|152218032\|gb\|ABS31450.1\| | MQRRKKKAQVVMFVHDLIICIYLFIVI | Medicago truncatula |
| NCR 313 | TTRKTDIRCRFYYDCPRLEYHFCECI | |
| | EDFCAYIRLN | |
| | (SEQ ID NO: 118) | |
| >gi\|152218030\|gb\|ABS31449.1\| | MAKVYMFVYALIIFVSPFLLATFRTRL | Medicago truncatula |
| NCR 312 | PCEKDDDCPEAFLPPVMKCVNRFC | |
| | QYEILE | |
| | (SEQ ID NO: 119) | |
| >gi\|152218028\|gb\|ABS31448.1\| | MIKQFSVCYIQMRRNMTTILKFPYIM | Medicago truncatula |
| NCR 310 | VICLLLLHVAAYEDFEKEIFDCKKDG | |
| | DCDHMCVTPGIPKCTGYVCFCFENL | |
| | (SEQ ID NO: 120) | |
| >gi\|152218026\|gb\|ABS31447.1\| | MQRSRNMTTIFKFAYIMIICVFLLNIA | Medicago truncatula |
| NCR 309 | AQEIENGIHPCKKNEDCNHMCVMP | |
| | GLPWCHENNLCFCYENAYGNTR | |
| | (SEQ ID NO: 121) | |
| >gi\|152218024\|gb\|ABS31446.1\| | MTIIIKFVNVLIIFLSLFHVAKNDDNKL | Medicago truncatula |
| NCR 304 | LLSFIEEGFLCFKDSDCPYNMCPSP | |
| | LKEMCYFIKCVCGVYGPIRERRLYQ | |
| | SHNPMIQ | |
| | (SEQ ID NO: 122) | |
| >gi\|152218022\|gb\|ABS31445.1\| | MRKNMTKILMIGYALMIFIFLSIAVSIT | Medicago truncatula |
| NCR 303 | GNLARASRKKPVDVIPCIYDHDCPR | |
| | KLYFLERCVGRVCKYL | |
| | (SEQ ID NO: 123) | |
| >gi\|152218020\|gb\|ABS31444.1\| | MAHKLVYAITLFIFLFLIANNIEDDIFCI | Medicago truncatula |
| NCR 301 | TDNDCPPNTLVQRYRCINGKCNLSF | |
| | VSYG | |
| | (SEQ ID NO: 124) | |
| >gi\|152218018\|gb\|ABS31443.1\| | MDETLKFVYILILFVSLCLVVADGVK | Medicago truncatula |
| NCR 300 | NINRECTQTSDCYKKYPFIPWGKVR | |
| | CVKGRCRLDM | |
| | (SEQ ID NO: 125) | |
| >gi\|152218016\|gb\|ABS31442.1\| | MAKIIKFVYVLAIFFSLFLVAKNVNG | Medicago truncatula |
| NCR 290 | WTCVEDSDCPANICQPPMQRMCFY | |
| | GECACVRSKFCT | |
| | (SEQ ID NO: 126) | |
| >gi\|152218014\|gb\|ABS31441.1\| | MVKIIKFVYFMTLFLSMLLVTTKEDG | Medicago truncatula |
| NCR 289 | SVECIANIDCPQIFMLPFVMRCINFR | |
| | CQIVNSEDT | |
| | (SEQ ID NO: 127) | |
| >gi\|152218012\|gb\|ABS31440.1\| | MDEILKFVYTLIIFFSLFFAANNVDANI | Medicago truncatula |
| NCR 286 | MNCQSTFDCPRDMCSHIRDVICIFK | |
| | KCKCAGGRYMPQVP | |
| | (SEQ ID NO: 128) | |
| >gi\|152218008\|gb\|ABS31438.1\| | MQRRKNMANNHMLIYAMIICLFPYL | Medicago truncatula |
| NCR 278 | VVTFKTAITCDCNEDCLNFFTPLDNL | |
| | KCIDNVCEVFM | |
| | (SEQ ID NO: 129) | |
| >gi\|152218006\|gb\|ABS31437.1\| | MVNILKFIYVIIFFILMFFVLIDVDGHV | Medicago truncatula |
| NCR 266 | LVECIENRDCEKGMCKFPFIVRCLM | |
| | DQCKCVRIHNLI | |
| | (SEQ ID NO: 130) | |
| >gi\|152218004\|gb\|ABS31436.1\| | MIIQFSIYYMQRRKLNMVEILKFSHA | Medicago truncatula |
| NCR 265 | LIIFLFLSALVTNANIFFCSTDEDCTW | |
| | NLCRQPWVQKCRLHMCSCEKN | |
| | (SEQ ID NO: 131) | |
| >gi\|152218002\|gb\|ABS31435.1\| | MDEVFKFVYVMIIFPFLILDVATNAEK | Medicago truncatula |
| NCR 263 | IRRCFNDAHCPPDMCTLGVIPKCSR | |
| | FTICIC | |
| | (SEQ ID NO: 132) | |
| >gi\|152218000\|gb\|ABS31434.1\| | MHRKPNMTKFFKFVYTMFILISLFLV | Medicago truncatula |
| NCR 244 | VTNANANNCTDTSDCSSNHCSYEG | |
| | VSLCMNGQCICIYE | |
| | (SEQ ID NO: 133) | |
| >gi\|152217998\|gb\|ABS31433.1\| | MQMKKMATILKFVYLIILLIYPLLVVTE | Medicago truncatula |
| NCR 239 | ESHYMKFSICKDDTDCPTLFCVLPN | |
| | VPKCIGSKCHCKLMVN | |
| | (SEQ ID NO: 134) | |
| >gi\|152217996\|gb\|ABS31432.1\| | MVETLRLFYIMILFVSLYLVVVDGVS | Medicago truncatula |
| NCR 237 | KLAQSCSEDFECYIKNPHAPFGQLR | |
| | CFEGYCQRLDKPT | |
| | (SEQ ID NO: 135) | |
| >gi\|152217994\|gb\|ABS31431.1\| | MTTFLKVAYIMIICVFVLHLAAQVDS | Medicago truncatula |
| NCR 228 | QKRLHGCKEDRDCDNICSVHAVTK | |
| | CIGNMCRCLANVK | |
| | (SEQ ID NO: 136) | |
| >gi\|152217992\|gb\|ABS31430.1\| | MRINRTPAIFKFVYTIIIYLFLLRVVAK | Medicago truncatula |
| NCR 224 | DLPFNICEKDEDCLEFCAHDKVAKC | |
| | MLNICFCF | |
| | (SEQ ID NO: 137) | |
| >gi\|152217990\|gb\|ABS31429.1\| | MAEILKILYVFIIFLSLILAVISQHPFTP | Medicago truncatula |
| NCR 221 | CETNADCKCRNHKRPDCLWHKCYC | |
| | Y | |
| | (SEQ ID NO: 138) | |
| >gi\|152217988\|gb\|ABS31428.1\| | MRKSMATILKFVYVIMLFIYSLFVIES | Medicago truncatula |
| NCR 217 | FGHRFLIYNNCKNDTECPNDCGPHE | |
| | QAKCILYACYCVE | |
| | (SEQ ID NO: 139) | |
| >gi\|152217986\|gb\|ABS31427.1\| | MNTILKFIFVVFLFLSIFLSAGNSKSY | Medicago truncatula |
| NCR 209 | GPCTTLQDCETHNWFEVCSCIDFEC | |
| | KCWSLL | |
| | (SEQ ID NO: 140) | |
| >gi\|152217984\|gb\|ABS31426.1\| | MAEIIKFVYIMILCVSLLLIAEASGKEC | Medicago truncatula |
| NCR 206 | VTDADCENLYPGNKKPMFCNNTGY | |
| | CMSLYKEPSRYM | |
| | (SEQ ID NO: 141) | |
| >gi\|152217982\|gb\|ABS31425.1\| | MAKIIKFVYIMILCVSLLLIVEAGGKEC | Medicago truncatula |
| NCR 201 | VTDVDCEKIYPGNKKPLICSTGYCYS | |
| | LYEEPPRYHK | |
| | (SEQ ID NO: 142) | |
| >gi\|152217980\|gb\|ABS31424.1\| | MAKVTKFGYIIIHFLSLFFLAMNVAG | Medicago truncatula |
| NCR 200 | GRECHANSHCVGKITCVLPQKPEC | |
| | WNYACVCYDSNKYR | |
| | (SEQ ID NO: 143) | |
| >gi\|152217978\|gb\|ABS31423.1\| | MAKIFNYVYALIMFLSLFLMGTSGMK | Medicago truncatula |
| NCR 192 | NGCKHTGHCPRKMCGAKTTKCRN | |
| | NKCQCV | |
| | (SEQ ID NO: 144) | |
| >gi\|152217976\|gb\|ABS31422.1\| | MTEILKFVCVMIIFISSFIVSKSLNGG | Medicago truncatula |
| NCR 189 | GKDKCFRDSDCPKHMCPSSLVAKCI | |
| | NRLCRCRRPELQVQLNP | |
| | (SEQ ID NO: 145) | |
| >gi\|152217974\|gb\|ABS31421.1\| | MAHIIMFVYALIYALIIFSSLFVRDGIP | Medicago truncatula |
| NCR 187 | CLSDDECPEMSHYSFKCNNKICEYD | |
| | LGEMSDDDYYLEMSRE | |
| | (SEQ ID NO: 146) | |
| >gi\|152217972\|gb\|ABS31420.1\| | MYREKNMAKTLKFVYVIVLFLSLFLA | Medicago truncatula |
| NCR 181 | AKNIDGRVSYNSFIALPVCQTAADC | |
| | PEGTRGRTYKCINNKCRYPKLLKPI | |
| | Q | |
| | (SEQ ID NO: 147) | |
| >gi\|152217970\|gb\|ABS31419.1\| | MAHIFNYVYALLVFLSLFLMVTNGIHI | Medicago truncatula |
| NCR 176 | GCDKDRDCPKQMCHLNQTPKCLKN | |
| | ICKCV | |
| | (SEQ ID NO: 148) | |
| >gi\|152217968\|gb\|ABS31418.1\| | MAEILKCFYTMNLFIFLIILPAKIREHI | Medicago truncatula |
| NCR 175 | QCVIDDDCPKSLNKLLIIKCINHVCQY | |
| | VGNLPDFASQIPKSTKMPYKGE | |
| | (SEQ ID NO: 149) | |
| >gi\|152217966\|gb\|ABS31417.1\| | MAYISRIFYVLIIFLSLFFVVINGVKSL | Medicago truncatula |
| NCR 173 | LLIKVRSFIPCQRSDDCPRNLCVDQII | |
| | PTCVWAKCKCKNYND | |
| | (SEQ ID NO: 150) | |
| >gi\|152217964\|gb\|ABS31416.1\| | MANVTKFVYIAIYFLSLFFIAKNDATA | Medicago truncatula |
| NCR 172 | TFCHDDSHCVTKIKCVLPRTPQCRN | |
| | EACGCYHSNKFR | |
| | (SEQ ID NO: 151) | |
| >gi\|152217962\|gb\|ABS31415.1\| | MGEIMKFVYVMIIYLFMFNVATGSEF | Medicago truncatula |
| NCR 171 | IFTKKLTSCDSSKDCRSFLCYSPKFP | |
| | VCKRGICECI | |
| | (SEQ ID NO: 152) | |
| >gi\|152217960\|gb\|ABS31414.1\| | MGEMFKFIYTFILFVHLFLVVIFEDIG | Medicago truncatula |
| NCR 169 | HIKYCGIVDDCYKSKKPLFKIWKCVE | |
| | NVCVLWYK | |
| | (SEQ ID NO: 153) | |
| >gi\|152217958\|gb\|ABS31413.1\| | MARTLKFVYSMILFLSLFLVANGLKIF | Medicago truncatula |
| NCR 165 | CIDVADCPKDLYPLLYKCIYNKCIVFT | |
| | RIPFPFDWI | |
| | (SEQ ID NO: 154) | |
| >gi\|152217956\|gb\|ABS31412.1\| | MANITKFVYIAILFLSLFFIGMNDAAIL | Medicago truncatula |
| NCR 159 | ECREDSHCVTKIKCVLPRKPECRNN | |
| | ACTCYKGGFSFHH | |
| | (SEQ ID NO: 155) | |
| >gi\|152217954\|gb\|ABS31411.1\| | MQRVKKMSETLKFVYVLILFISIFHVV | Medicago truncatula |
| NCR 147 | IVCDSIYFPVSRPCITDKDCPNMKHY | |
| | KAKCRKGFCISSRVR | |
| | (SEQ ID NO: 156) | |
| >gi\|152217952\|gb\|ABS31410.1\| | MQIRKIMSGVLKFVYAIILFLFLFLVA | Medicago truncatula |
| NCR 146 | REVGGLETIECETDGDCPRSMIKM | |
| | WNKNYRHKCIDGKCEWIKKLP | |
| | (SEQ ID NO: 157) | |
| >gi\|152217950\|gb\|ABS31409.1\| | MFVYDLILFISLILVVTGINAEADTSC | Medicago truncatula |
| NCR 145 | HSFDDCPWVAHHYRECIEGLCAYRI | |
| | LY | |
| | (SEQ ID NO: 158) | |
| >gi\|152217948\|gb\|ABS31408.1\| | MQRRKKSMAKMLKFFFAIILLLSLFL | Medicago truncatula |
| NCR 144 | VATEVGGAYIECEVDDDCPKPMKN | |
| | SHPDTYYKCVKHRCQWAWK | |
| | (SEQ ID NO: 159) | |
| >gi\|152217946\|gb\|ABS31407.1\| | MFVYTLIIFLFPSHVITNKIAIYCVSDD | Medicago truncatula |
| NCR 140 | DCLKTFTPLDLKCVDNVCEFNLRCK | |
| | GKCGERDEKFVFLKALKKMDQKLVL | |
| | EEQGNAREVKIPKKLLFDRIQVPTPA | |
| | TKDQVEEDDYDDDDEEEEEEEDDV | |
| | DMWFHLPDVVCH | |
| | (SEQ ID NO: 160) | |
| >gi\|152217944\|gb\|ABS31406.1\| | MAKFSMFVYALINFLSLFLVETAITNI | Medicago truncatula |
| NCR 138 | RCVSDDDCPKVIKPLVMKCIGNYCY | |
| | FFMIYEGP | |
| | (SEQ ID NO: 161) | |
| >gi\|152217942\|gb\|ABS31405.1\| | MAHKFVYAIILFIFLFLVAKNVKGYVV | Medicago truncatula |
| NCR 136 | CRTVDDCPPDTRDLRYRCLNGKCK | |
| | SYRLSYG | |
| | (SEQ ID NO: 162) | |
| >gi\|152217940\|gb\|ABS31404.1\| | MQRKKNMGQILIFVFALINFLSPILVE | Medicago truncatula |
| NCR 129 | MTTTTIPCTFIDDCPKMPLVVKCIDN | |
| | FCNYFEIK | |
| | (SEQ ID NO: 163) | |
| >gi\|152217938\|gb\|ABS31403.1\| | MAQTLMLVYALIIFTSLFLVVISRQTD | Medicago truncatula |
| NCR 128 | IPCKSDDACPRVSSHHIECVKGFCT | |
| | YVVKLD | |
| | (SEQ ID NO: 164) | |
| >gi\|152217936\|gb\|ABS31402.1\| | MLRRKNTVQILMFVSALLIYIFLFLVIT | Medicago truncatula |
| NCR 127 | SSANIPCNSDSDCPWKIYYTYRCND | |
| | GFCVYKSIDPSTIPQYMTDLIFPR | |
| | (SEQ ID NO: 165) | |
| >gi\|152217934\|gb\|ABS31401.1\| | MAVILKFVYIMIIFLFLLYVVNGTRCN | Medicago truncatula |
| NCR 122 | RDEDCPFICTGPQIPKCVSHICFCLS | |
| | SGKEAY | |
| | (SEQ ID NO: 166) | |
| >gi\|152217932\|gb\|ABS31400.1\| | MDAILKFIYAMFLFLFLFVTTRNVEAL | Medicago truncatula |
| NCR 121 | FECNRDFVCGNDDECVYPYAVQCI | |
| | HRYCKCLKSRN | |
| | (SEQ ID NO: 167) | |
| >gi\|152217930\|gb\|ABS31399.1\| | MQIGRKKMGETPKLVYVIILFLSIFLC | Medicago truncatula |
| NCR 119 | TNSSFSQMINFRGCKRDKDCPQFR | |
| | GVNIRCRSGFCTPIDS | |
| | (SEQ ID NO: 168) | |
| >gi\|152217928\|gb\|ABS31398.1\| | MQMRKNMAQILFYVYALLILFSPFLV | Medicago truncatula |
| NCR 118 | ARIMVVNPNNPCVTDADCQRYRHK | |
| | LATRMVCNIGFCLMDFTHDPYAPSL | |
| | P | |
| | (SEQ ID NO: 169) | |
| >gi\|152217926\|gb\|ABS31397.1\| | MYVYYIQMGKNMAQRFMFIYALIIFL | Medicago truncatula |
| NCR 111 | SQFFVVINTSDIPNNSNRNSPKEDVF | |
| | CNSNDDCPTILYYVSKCVYNFCEYVV | |
| | (SEQ ID NO: 170) | |
| >gi\|152217924\|gb\|ABS31396.1\| | MAKIVNFVYSMIIFVSLFLVATKGGS | Medicago truncatula |
| NCR 103 | KPFLTRPYPCNTGSDCPQNMCPPG | |
| | YKPGCEDGYCNHCYKRW | |
| | (SEQ ID NO: 171) | |
| >gi\|152217922\|gb\|ABS31395.1\| | MVRTLKFVYVIILILSLFLVAKGGGKK | Medicago truncatula |
| NCR 101 | IYCENAASCPRLMYPLVYKCLDNKC | |
| | VKFMMKSRFV | |
| | (SEQ ID NO: 172) | |
| >gi\|152217920\|gb\|ABS31394.1\| | MARTLKFVYAVILFLSLFLVAKGDDV | Medicago truncatula |
| NCR 96 | KIKCVVAANCPDLMYPLVYKCLNGIC | |
| | VQFTLTFPFV | |
| | (SEQ ID NO: 173) | |
| >gi\|152217918\|gb\|ABS31393.1\| | MSNTLMFVITFIVLVTLFLGPKNVYA | Medicago truncatula |
| NCR 94 | FQPCVTTADCMKTLKTDENIWYECI | |
| | NDFCIPFPIPKGRK | |
| | (SEQ ID NO: 174) | |
| >gi\|152217916\|gb\|ABS31392.1\| | MKRVVNMAKIVKYVYVIIIFLSLFLVA | Medicago truncatula |
| NCR 93 | TKIEGYYYKCFKDSDCVKLLCRIPLR | |
| | PKCMYRHICKCKVVLTQNNYVLT | |
| | (SEQ ID NO: 175) | |
| >gi\|152217914\|gb\|ABS31391.1\| | MKRGKNMSKILKFIYATLVLYLFLVV | Medicago truncatula |
| NCR 90 | TKASDDECKIDGDCPISWQKFHTYK | |
| | CINQKCKWVLRFHEY | |
| | (SEQ ID NO: 176) | |
| >gi\|152217912\|gb\|ABS31390.1\| | MAKTLNFMFALILFISLFLVSKNVAIDI | Medicago truncatula |
| NCR 88 | FVCQTDADCPKSELSMYTWKCIDN | |
| | ECNLFKVMQQMV | |
| | (SEQ ID NO: 177) | |
| >gi\|152217910\|gb\|ABS31389.1\| | MANTHKLVSMILFIFLFLVANNVEGY | Medicago truncatula |
| NCR 86 | VNCETDADCPPSTRVKRFKCVKGE | |
| | CRWTRMSYA | |
| | (SEQ ID NO: 178) | |
| >gi\|152217908\|gb\|ABS31388.1\| | MAHFLMFVYALITCLSLFLVEMGHLS | Medicago truncatula |
| NCR 77 | IHCVSVDDCPKVEKPITMKCINNYCK | |
| | YFVDHKL | |
| | (SEQ ID NO: 179) | |
| >gi\|152217906\|gb\|ABS31387.1\| | MNQIPMFGYTLIIFFSLFPVITNGDRI | Medicago truncatula |
| NCR 76 | PCVTNGDCPVMRLPLYMRCITYSCE | |
| | LFFDGPNLCAVERI | |
| | (SEQ ID NO: 180) | |
| >gi\|152217904\|gb\|ABS31386.1\| | MRKDMARISLFVYALIIFFSLFFVLTN | Medicago truncatula |
| NCR 74 | GELEIRCVSDADCPLFPLPLHNRCID | |
| | DVCHLFTS | |
| | (SEQ ID NO: 181) | |
| >gi\|152217902\|gb\|ABS31385.1\| | MAQILMFVYFLIIFLSLFLVESIKIFTE | Medicago truncatula |
| NCR 68 | HRCRTDADCPARELPEYLKCQGGM | |
| | CRLLIKKD | |
| | (SEQ ID NO: 182) | |
| >gi\|152217900\|gb\|ABS31384.1\| | MARVISLFYALIIFLFLFLVATNGDLS | Medicago truncatula |
| NCR 65 | PCLRSGDCSKDECPSHLVPKCIGLT | |
| | CYCI | |
| | (SEQ ID NO: 183) | |
| >gi\|152217898\|gb\|ABS31383.1\| | MQRRKNMAQILLFAYVFIISISLFLVV | Medicago truncatula |
| NCR 62 | TNGVKIPCVKDTDCPTLPCPLYSKC | |
| | VDGFCKMLSI | |
| | (SEQ ID NO: 184) | |
| >gi\|152217896\|gb\|ABS31382.1\| | MNHISKFVYALIIFLSVYLVVLDGRPV | Medicago truncatula |
| NCR 57 | SCKDHYDCRRKVKIVGCIFPQEKPM | |
| | CINSMCTCIREIVP | |
| | (SEQ ID NO: 185) | |
| >gi\|152217894\|gb\|ABS31381.1\| | MKSQNHAKFISFYKNDLFKIFQNND | Medicago truncatula |
| NCR 56 | SHFKVFFALIIFLYTYLHVTNGVFVSC | |
| | NSHIHCRVNNHKIGCNIPEQYLLCVN | |
| | LFCLWLDY | |
| | (SEQ ID NO: 186) | |
| >gi\|152217892\|gb\|ABS31380.1\| | MTYISKVVYALIIFLSIYVGVNDCMLV | Medicago truncatula |
| NCR 54 | TCEDHFDCRQNVQQVGCSFREIPQ | |
| | CINSICKCMKG | |
| | (SEQ ID NO: 187) | |
| >gi\|152217890\|gb\|ABS31379.1\| | MTHISKFVFALIIFLSIYVGVNDCKRIP | Medicago truncatula |
| NCR 53 | CKDNNDCNNNWQLLACRFEREVPR | |
| | CINSICKCMPM | |
| | (SEQ ID NO: 188) | |
| >gi\|152217888\|gb\|ABS31378.1\| | MVQTPKLVYVIVLLLSIFLGMTICNSS | Medicago truncatula |
| NCR 43 | FSHFFEGACKSDKDCPKLHRSNVR | |
| | CRKGQCVQI | |
| | (SEQ ID NO: 189) | |
| >gi\|152217886\|gb\|ABS31377.1\| | MTKILMLFYAMIVFHSIFLVASYTDEC | Medicago truncatula |
| NCR 28 | STDADCEYILCLFPIIKRCIHNHCKCV | |
| | PMGSIEPMSTIPNGVHKFHIINN | |
| | (SEQ ID NO: 190) | |
| >gi\|152217884\|gb\|ABS31376.1\| | MAKTLNFVCAMILFISLFLVSKNVAL | Medicago truncatula |
| NCR 26 | YIIECKTDADCPISKLNMYNWRCIKS | |
| | SCHLYKVIQFMV | |
| | (SEQ ID NO: 191) | |
| >gi\|152217882\|gb\|ABS31375.1\| | MQKEKNMAKTFEFVYAMIIFILLFLVE | Medicago truncatula |
| NCR 24 | NNFAAYIIECQTDDDCPKSQLEMFA | |
| | WKCVKNGCHLFGMYEDDDDP | |
| | (SEQ ID NO: 192) | |
| >gi\|152217880\|gb\|ABS31374.1\| | MAATRKFIYVLSHFLFLFLVTKITDAR | Medicago truncatula |
| NCR 21 | VCKSDKDCKDIIIYRYILKCRNGECV | |
| | KIKI | |
| | (SEQ ID NO: 193) | |
| >gi\|152217878\|gb\|ABS31373.1\| | MQRLDNMAKNVKFIYVIILLLFIFLVII | Medicago truncatula |
| NCR 20 | VCDSAFVPNSGPCTTDKDCKQVKG | |
| | YIARCRKGYCMQSVKRTWSSYSR | |
| | (SEQ ID NO: 194) | |
| >gi\|152217876\|gb\|ABS31372.1\| | MKFIYIMILFLSLFLVQFLTCKGLTVP | Medicago truncatula |
| NCR 19 | CENPTTCPEDFCTPPMITRCINFICL | |
| | CDGPEYAEPEYDGPEPEYDHKGDF | |
| | LSVKPKIINENMMMRERHMMKEIEV | |
| | (SEQ ID NO: 195) | |
| >gi\|152217874\|gb\|ABS31371.1\| | MAQFLMFIYVLIIFLYLFYVEAAMFEL | Medicago truncatula |
| NCR 12 | TKSTIRCVTDADCPNVVKPLKPKCV | |
| | DGFCEYT | |
| | (SEQ ID NO: 196) | |
| >gi\|152217872\|gb\|ABS31370.1\| | MKMRIHMAQIIMFFYALIIFLSPFLVD | Medicago truncatula |
| NCR 10 | RRSFPSSFVSPKSYTSEIPCKATRD | |
| | CPYELYYETKCVDSLCTY | |
| | (SEQ ID NO: 197) | |

Any NCR peptide known in the art is suitable for use in the methods or compositions described herein. NCR peptide-producing plants include but are not limited to Pisum sativum (pea), Astragalus sinicus (IRLC legumes), Phaseolus vulgaris (bean), Vigna unguiculata (cowpea), Medicago truncatula (barrelclover), and Lotus japonicus. For example, over 600 potential NCR peptides are predicted from the M. truncatula genome sequence and almost 150 different NCR peptides have been detected in cells isolated from root nodules by mass spectrometry.

The NCR peptides described herein may be mature or immature NCR peptides. Immature NCR peptides have a C-terminal signal peptide that is required for translocation into the endoplasmic reticulum and cleaved after translocation. The N-terminus of a NCR peptide includes a signal peptide, which may be cleavable, for targeting to a secretory pathway. NCR peptides are generally small peptides with disulfide bridges that stabilize their structure. Mature NCR peptides have a length in the range of about 20 to about 60 amino acids, about 25 to about 55 amino acids, about 30 to about 50 amino acids, about 35 to about 45 amino acids, or any range therebetween. NCR peptides may include a conserved sequence of cysteine residues with the rest of the peptide sequence highly variable. NCR peptides generally have about four or eight cysteines.

NCR peptides may be anionic, neutral, or cationic. In some instances, synthetic cationic NCR peptides having a pl greater than about eight possess antimicrobial activities. For example, NCR247 (pl=10.15) (RNGCIVDPRCPYQQCRRPLYCRRR; SEQ ID NO: 198) and NCR335 (pl=11.22) are both effective against gram-negative and gram-positive bacteria as well as fungi. In some instances, neutral and/or anionic NCR peptides, such as NCR001 (MAQFLLFVYSLIIFLSLFFGEAAFERTETRMLTIPCTSDDNCPKVISPCHTKCFDGFCGWYIEGSYEGP; SEQ ID NO: 199), do not possess antimicrobial activities at a pl greater than about 8.

In some instances, the NCR peptide is effective to kill bacteria. In some instances, the NCR peptide is effective to kill S. meliloti, Xenorhabdus spp, Photorhabdus spp, Candidatus spp, Buchnera spp, Blattabacterium spp, Baumania spp, Wigglesworthia spp, Wolbachia spp, Rickettsia spp, Orientia spp, Sodalis spp, Burkholderia spp, Cupriavidus spp, Frankia spp, Snirhizobium spp, Streptococcus spp, Wolinella spp, Xylella spp, Erwinia spp, Agrobacterium spp, Bacillus spp, Paenibacillus spp, Streptomyces spp, Micrococcus spp, Corynebacterium spp, Acetobacter spp, Cyanobacteria spp, Salmonella spp, Rhodococcus spp, Pseudomonas spp, Lactobacillus spp, Enterococcus spp, Alcaligenes spp, Klebsiella spp, Paenibacillus spp, Arthrobacter spp, Corynebacterium spp, Brevibacterium spp, Thermus spp, Pseudomonas spp, Clostridium spp, or Escherichia spp.

In some instances, the NCR peptide is a functionally active variant of a NCR peptide described herein. In some instances, the variant of the NCR peptide has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a NCR peptide described herein or naturally derived NCR peptide.

In some instances, the NCR peptide may be bioengineered to modulate its bioactivity, e.g., increase or decrease or regulate, or to specify a target microorganism. In some instances, the NCR peptide is produced by the translational machinery (e.g. a ribosome, etc.) of a cell. In some instances, the NCR peptide is chemically synthesized. In some instances, the NCR peptide is derived from a polypeptide precursor. The polypeptide precursor can undergo cleavage (for example, processing by a protease) to yield the NCR peptide itself. As such, in some instances, the NCR peptide is produced from a precursor polypeptide. In some instances, the NCR peptide includes a polypeptide that has undergone post-translational modifications, for example, cleavage, or the addition of one or more functional groups.

The NCR peptide described herein may be formulated in a composition for any of the uses described herein. The compositions disclosed herein may include any number or type of NCR peptides, such as at least about any one of 1 NCR peptide, 2, 3, 4, 5, 10, 15, 20, 30, 40, 50, 100, or more NCR peptides. A suitable concentration of each NCR peptide in the composition depends on factors such as efficacy, stability of the NCR peptide, number of distinct NCR peptide, the formulation, and methods of application of the composition. In some instances, each NCR peptide in a liquid composition is from about 0.1 ng/mL to about 100 mg/mL. In some instances, each NCR peptide in a solid composition is from about 0.1 ng/g to about 100 mg/g. In some instances, wherein the composition includes at least two types of NCR peptides, the concentration of each type of NCR peptide may be the same or different.

A modulating agent including a NCR peptide as described herein can be contacted with the target host in an amount and for a time sufficient to: (a) reach a target level (e.g., a predetermined or threshold level) of NCR peptide concentration inside a target host; (b) reach a target level (e.g., a predetermined or threshold level) of NCR peptide concentration inside a target host gut; (c) reach a target level (e.g., a predetermined or threshold level) of NCR peptide concentration inside a target host bacteriocyte; (d) modulate the level, or an activity, of one or more microorganism (e.g., endosymbiont) in the target host; or/and (e) modulate fitness of the target host.

(e) Bacteriocyte Regulatory Peptides

The modulating agent described herein may include a bacteriocyte regulatory peptide (BRP). BRPs are peptides expressed in the bacteriocytes of insects. These genes are expressed first at a developmental time point coincident with the incorporation of symbionts and their bacteriocyte-specific expression is maintained throughout the insect's life. In some instances, the BRP has a hydrophobic amino terminal domain, which is predicted to be a signal peptide. In addition, some BRPs have a cysteine-rich domain. In some instances, the bacteriocyte regulatory peptide is a bacteriocyte-specific cysteine rich (BCR) protein. Bacteriocyte regulatory peptides have a length between about 40 and 150 amino acids. In some instances, the bacteriocyte regulatory peptide has a length in the range of about 45 to about 145, about 50 to about 140, about 55 to about 135, about 60 to about 130, about 65 to about 125, about 70 to about 120, about 75 to about 115, about 80 to about 110, about 85 to about 105, or any range therebetween. Non-limiting examples of BRPs and their activities are listed in Table 8.

TABLE 8
Examples of Bacteriocyte Regulatory Peptides

| Name | Peptide Sequence |
|---|---|
| Bacteriocyte-specific cysteine rich | MKLLHGFLIIMLTMHLSIQYAYGGPFLTKYLCDRVCHKLC |
| proteins BCR family, peptide BCR1 | GDEFVCSCIQYKSLKGLWFPHCPTGKASVVLHNFLTSP |
| | (SEQ ID NO: 200) |
| Bacteriocyte-specific cysteine rich | MKLLYGFLIIMLTIHLSVQYFESPFETKYNCDTHCNKLCGK |
| proteins BCR family, peptide BCR2 | IDHCSCIQYHSMEGLWFPHCRTGSAAQMLHDFLSNP |
| | (SEQ ID NO: 201) |
| Bacteriocyte-specific cysteine rich | MSVRKNVLPTMFVVLLIMSPVTPTSVFISAVCYSGCGSLA |
| proteins BCR family, peptide BCR3 | LVCFVSNGITNGLDYFKSSAPLSTSETSCGEAFDTCTDH |
| | CLANFKF |
| | (SEQ ID NO: 202) |
| Bacteriocyte-specific cysteine rich | MRLLYGFLIIMLTIYLSVQDFDPTEFKGPFPTIEICSKYCAV |
| proteins BCR family, peptide BCR4 | VCNYTSRPCYCVEAAKERDQWFPYCYD |
| | (SEQ ID NO: 203) |
| Bacteriocyte-specific cysteine rich | MRLLYGFLIIMLTIHLSVQDIDPNTLRGPYPTKEICSKYCEY |
| proteins BCR family, peptide BCR5 | NVVCGASLPCICVQDARQLDHWFACCYDGGPEMLM |
| | (SEQ ID NO: 204) |
| Secreted proteins SP family, peptide | MKLFVVVVLVAVGIMFVFASDTAAAPTDYEDTNDMISLSS |
| SP1 | LVGDNSPYVRVSSADSGGSSKTSSKNPILGLLKSVIKLLT |
| | KIFGTYSDAAPAMPPIPPALRKNRGMLA |
| | (SEQ ID NO: 205) |
| Secreted proteins SP family, peptide | MVACKVILAVAVVFVAAVQGRPGGEPEWAAPIFAELKSV |
| SP2 | SDNITNLVGLDNAGEYATAAKNNLNAFAESLKTEAAVFSK |
| | SFEGKASASDVFKESTKNFQAVVDTYIKNLPKDLTLKDFT |
| | EKSEQALKYMVEHGTEITKKAQGNTETEKEIKEFFKKQIE |
| | NLIGQGKALQAKIAEAKKA |
| | (SEQ ID NO: 206) |
| Secreted proteins SP family, peptide | MKTSSSKVFASCVAIVCLASVANALPVQKSVAATTENPIV |
| SP3 | EKHGCRAHKNLVRQNVVDLKTYDSMLITNEVVQKQSNE |
| | VQSSEQSNEGQNSEQSNEGQNSEQSNEVQSSEHSNEG |
| | QNSKQSNEGQNSEQSNEVQSSEHSNEGQNSEQSNEVQ |
| | SSEHSNEGQNSKQSNEGQNSKQSNEVQSSEHWNEGQ |
| | NSKQSNEDQNSEQSNEGQNSKQSNEGQNSKQSNEDQ |
| | NSEQSNEGQNSKQSNEVQSSEQSNEGQNSKQSNEGQS |
| | SEQSNEGQNSKQSNEVQSPEEHYDLPDPESSYESEETK |
| | GSHESGDDSEHR |
| | (SEQ ID NO: 207) |
| Secreted proteins SP family, peptide | MKTIILGLCLFGALFWSTQSMPVGEVAPAVPAVPSEAVP |
| SP4 | QKQVEAKPETNAASPVSDAKPESDSKPVDAEVKPTVSEV |
| | KAESEQKPSGEPKPESDAKPVVASESKPESDPKPAAVVE |
| | SKPENDAVAPETNNDAKPENAAAPVSENKPATDAKAETE |
| | LIAQAKPESKPASDLKAEPEAAKPNSEVPVALPLNPTETK |
| | ATQQSVETNQVEQAAPAAAQADPAAAPAADPAPAPAAA |
| | PVAAEEAKLSESAPSTENKAAEEPSKPAEQQSAKPVEDA |
| | VPAASEISETKVSPAVPAVPEVPASPSAPAVADPVSAPEA |
| | EKNAEPAKAANSAEPAVQSEAKPAEDIQKSGAVVSAENP |
| | KPVEEQKPAEVAKPAEQSKSEAPAEAPKPTEQSAAEEPK |
| | KPESANDEKKEQHSVNKRDATKEKKPTDSIMKKQKQKK |
| | AN |
| | (SEQ ID NO: 208) |
| Secreted proteins SP family, peptide | MNGKIVLCFAVVFIGQAMSAATGTTPEVEDIKKVAEQMS |
| SP5a | QTFMSVANHLVGITPNSADAQKSIEKIRTIMNKGFTDMET |
| | EANKMKDIVRKNADPKLVEKYDELEKELKKHLSTAKDMF |
| | EDKVVKPIGEKVELKKITENVIKTTKDMEATMNKAIDGFKK |
| | Q |
| | (SEQ ID NO: 209) |
| Secreted proteins SP family, peptide | MHLFLALGLFIVCGMVDATFYNPRSQTFNQLMERRQRSI |
| SP6 | PIPYSYGYHYNPIEPSINVLDSLSEGLDSRINTFKPIYQNV |
| | KMSTQDVNSVPRTQYQPKNSLYDSEYISAKDIPSLFPEE |
| | DSYDYKYLGSPLNKYLTRPSTQESGIAINLVAIKETSVFDY |
| | GFPTYKSPYSSDSVWNFGSKIPNTVFEDPQSVESDPNTF |
| | KVSSPTIKIVKLLPETPEQESIITTTKNYELNYKTTQETPTE |
| | AELYPITSEEFQTEDEWHPMVPKENTTKDESSFITTEEPL |
| | TEDKSNSITIEKTQTEDESNSIEFNSIRTEEKSNSITTEENQ |
| | KEDDESMSTTSQETTTAFNLNDTFDTNRYSSSHESLMLR |
| | IRELMKNIADQQNKSQFRTVDNIPAKSQSNLSSDESTNQ |
| | QFEPQLVNGADTYK |
| | (SEQ ID NO: 210) |
| Colepotericin A, ColA peptide | MTRTMLFLACVAALYVCISATAGKPEEFAKLSDEAPSND |
| | QAMYESIQRYRRFVDGNRYNGGQQQQQQPKQWEVRP |
| | DLSRDQRGNTKAQVEINKKGDNHDINAGWGKNINGPDS |
| | HKDTWHVGGSVRW |
| | (SEQ ID NO: 211) |
| RlpA type I | MKETTVVWAKLFLILIILAKPLGLKAVNECKRLGNNSCRSH |
| | GECCSGFCFIEPGWALGVCKRLGTPKKSDDSNNGKNIEK |
| | NNGVHERIDDVFERGVCSYYKGPSITANGDVFDENEMTA |
| | AHRTLPFNTMVKVEGMGTSVVVKINDRKTAADGKVMLLS |
| | RAAAESLNIDENTGPVQCQLKFVLDGSGCTPDYGDTCVL |
| | HHECCSQNCFREMFSDKGFCLPK (SEQ ID NO: 212) |

In some instances, the BRP alters the growth and/or activity of one or more bacteria resident in the bacteriocyte of the host. In some instances, the BRP may be bioengineered to modulate its bioactivity (e.g., increase, decrease, or regulate) or to specify a target microorganism. In some instances, the BRP is produced by the translational machinery (e.g. a ribosome, etc.) of a cell. In some instances, the BRP is chemically synthesized. In some instances, the BRP is derived from a polypeptide precursor. The polypeptide precursor can undergo cleavage (for example, processing by a protease) to yield the polypeptide of the BRP itself. As such, in some instances, the BRP is produced from a precursor polypeptide. In some instances, the BRP includes a polypeptide that has undergone post-translational modifications, for example, cleavage, or the addition of one or more functional groups.

Functionally active variants of the BRPs as described herein are also useful in the compositions and methods described herein. In some instances, the variant of the BRP has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a BRP described herein or naturally derived BRP.

The BRP described herein may be formulated in a composition for any of the uses described herein. The compositions disclosed herein may include any number or type (e.g., classes) of BRPs, such as at least about any one of 1 BRP, 2, 3, 4, 5, 10, 15, 20, or more BRPs. A suitable concentration of each BRP in the composition depends on factors such as efficacy, stability of the BRP, number of distinct BRP, the formulation, and methods of application of the composition. In some instances, each BRP in a liquid composition is from about 0.1 ng/mL to about 100 mg/mL. In some instances, each BRP in a solid composition is from about 0.1 ng/g to about 100 mg/g. In some instances, wherein the composition includes at least two types of BRPs, the concentration of each type of BRP may be the same or different.

A modulating agent including a BRP as described herein can be contacted with the target host in an amount and for a time sufficient to: (a) reach a target level (e.g., a predetermined or threshold level) of BRP concentration inside a target host; (b) reach a target level (e.g., a predetermined or threshold level) of BRP concentration inside a target host gut; (c) reach a target level (e.g., a predetermined or threshold level) of BRP concentration inside a target host bacteriocyte; (d) modulate the level, or an activity, of one or more microorganism (e.g., endosymbiont) in the target host; or/and (e) modulate fitness of the target host.

iii. Small Molecules

Numerous small molecules (e.g., an antibiotic or a metabolite) may be used in the compositions and methods described herein. In some instances, an effective concentration of any small molecule described herein may alter the level, activity, or metabolism of one or more microorganisms (as described herein) resident in a host, the alteration resulting in a decrease in the host's fitness.

A modulating agent comprising a small molecule as described herein can be contacted with the target host in an amount and for a time sufficient to: (a) reach a target level (e.g., a predetermined or threshold level) of a small molecule concentration inside a target host; (b) reach a target level (e.g., a predetermined or threshold level) of small molecule concentration inside a target host gut; (c) reach a target level (e.g., a predetermined or threshold level) of a small molecule concentration inside a target host bacteriocyte; (d) modulate the level, or an activity, of one or more microorganism (e.g., endosymbiont) in the target host; or/and (e) modulate fitness of the target host.

The small molecules discussed hereinafter, namely antibiotics and secondary metabolites, can be used to alter the level, activity, or metabolism of target microorganisms as indicated in the sections for decreasing the fitness of insects, such as aphids.

(a) Antibiotics

The modulating agent described herein may include an antibiotic. Any antibiotic known in the art may be used. Antibiotics are commonly classified based on their mechanism of action, chemical structure, or spectrum of activity.

The antibiotic described herein may target any bacterial function or growth processes and may be either bacteriostatic (e.g., slow or prevent bacterial growth) or bactericidal (e.g., kill bacteria). In some instances, the antibiotic is a bactericidal antibiotic. In some instances, the bactericidal antibiotic is one that targets the bacterial cell wall (e.g., penicillins and cephalosporins); one that targets the cell membrane (e.g., polymyxins); or one that inhibits essential bacterial enzymes (e.g., rifamycins, lipiarmycins, quinolones, and sulfonamides). In some instances, the bactericidal antibiotic is an aminoglycoside. In some instances, the antibiotic is a bacteriostatic antibiotic. In some instances the bacteriostatic antibiotic targets protein synthesis (e.g., macrolides, lincosamides, and tetracyclines). Additional classes of antibiotics that may be used herein include cyclic lipopeptides (such as daptomycin), glycylcyclines (such as tigecycline), oxazolidinones (such as linezolid), or lipiarmycins (such as fidaxomicin). Examples of antibiotics include rifampicin, ciprofloxacin, doxycycline, ampicillin, and polymyxin B. Other non-limiting examples of antibiotics are found in Table 9.

TABLE 9
Examples of Antibiotics

| Antibiotics | Action |
|---|---|
| Penicillins, cephalosporins, vancomycin | Cell wall synthesis |
| Polymixin, gramicidin | Membrane active agent, disrupt |
| | cell membrane |
| Tetracyclines, macrolides, chloramphenicol, | Inhibit protein synthesis |
| clindamycin, spectinomycin | |
| Sulfonamides | Inhibit folate-dependent |
| | pathways |
| Ciprofloxacin | Inhibit DNA-gyrase |
| Isoniazid, rifampicin, pyrazinamide, | Antimycobacterial agents |
| ethambutol, (myambutol)l, streptomycin | |

The antibiotic described herein may have any level of target specificity (e.g., narrow- or broad-spectrum). In some instances, the antibiotic is a narrow-spectrum antibiotic, and thus targets specific types of bacteria, such as gram-negative or gram-positive bacteria. Alternatively, the antibiotic may be a broad-spectrum antibiotic that targets a wide range of bacteria.

The antibiotics described herein may be formulated in a composition for any of the uses described herein. The compositions disclosed herein may include any number or type (e.g., classes) of antibiotics, such as at least about any one of 1 antibiotic, 2, 3, 4, 5, 10, 15, 20, or more antibiotics (e.g., a combination of rifampicin and doxycycline, or a combination of ampicillin and rifampicin). A suitable concentration of each antibiotic in the composition depends on factors such as efficacy, stability of the antibiotic, number of distinct antibiotics, the formulation, and methods of application of the composition. In some instances, wherein the composition includes at least two types of antibiotics, the concentration of each type of antibiotic may be the same or different.

A modulating agent including an antibiotic as described herein can be contacted with the target host in an amount and for a time sufficient to: (a) reach a target level (e.g., a predetermined or threshold level) of antibiotic concentration inside a target host; (b) reach a target level (e.g., a predetermined or threshold level) of antibiotic concentration inside a target host gut; (c) reach a target level (e.g., a predetermined or threshold level) of antibiotic concentration inside a target host bacteriocyte; (d) modulate the level, or an activity, of one or more microorganism (e.g., endosymbiont) in the target host; or/and (e) modulate fitness of the target host.

As illustrated by Examples 6 and 11, antibiotics (e.g., rifampicin) can be used as modulating agents that target an endosymbiotic bacterium, such as a Buchnera spp., in an insect host, such as aphids, to decrease the fitness of the host (e.g., as outlined herein). As further illustrated by Example 7, antibiotics such as oxytetracycline can be used as modulating agents that target an endosymbiotic bacterium, such as a Bacillus spp., in an insect host, such as Varroa mites, to decrease the fitness of the host (e.g., as outlined herein). As yet further illustrated by Example 23, antibiotics (e.g., ciprofloxacin) can be used as modulating agents that target an endosymbiotic bacterium, such as a Sitophilus spp., in an insect host, such as weevils, to decrease the fitness of the host (e.g., as outlined herein). As also illustrated by Example 24, antibiotics (e.g., rifampicin or doxycline) can be used as modulating agents that target an endosymbiotic bacterium in an insect host, such as mites, to decrease the fitness of the host (e.g., as outlined herein).

(b) Secondary Metabolites

In some instances, the modulating agent of the compositions and methods described herein includes a secondary metabolite. Secondary metabolites are derived from organic molecules produced by an organism. Secondary metabolites may act (i) as competitive agents used against bacteria, fungi, amoebae, plants, insects, and large animals; (ii) as metal transporting agents; (iii) as agents of symbiosis between microbes and plants, insects, and higher animals; (iv) as sexual hormones; and (v) as differentiation effectors. Non-limiting examples of secondary metabolites are found in Table 10.

TABLE 10
Examples of Secondary Metabolites

| Phenyl- | | | | | |
|---|---|---|---|---|---|
| propanoids | Alkaloids | Terpenoids | Quinones | Steroids | Polyketides |
| Anthocyanins | Acridines | Carotenes | Anthro- | Cardiac | Erythromycin |
| | | | quinones | | |
| Coumarins | Betalaines | Monoterpenes | Bezo- | Glycosides | Lovastatin and |
| | | | quinones | | other statins |
| Flavonoids | Quinolo- | Sesquiterpenes | Naphtho- | Pregnenolone | Discoder-molide |
| | zidines | | quinones | | |
| Hydroxy- | Furono- | Diterpenes | | Derivatives | Aflatoxin B1 |
| cinnamoyl | quinones | | | | |
| Derivatives | Herring- | Triterpenes | | | Avermectins |
| | tonines | | | | |
| Isoflavonoids | Isoquino- | | | | Nystatin |
| | lines | | | | |
| Lignans | Indoles | | | | Rifamycin |
| Phenolenones | Purines | | | | |
| Proantho- | Pyridines | | | | |
| cyanidins | | | | | |
| Stilbenes | Tropane | | | | |
| Tanins | Alkaloids | | | | |

The secondary metabolite used herein may include a metabolite from any known group of secondary metabolites. For example, secondary metabolites can be categorized into the following groups: alkaloids, terpenoids, flavonoids, glycosides, natural phenols (e.g., gossypol acetic acid), enals (e.g., trans-cinnamaldehyde), phenazines, biphenols and dibenzofurans, polyketides, fatty acid synthase peptides, nonribosomal peptides, ribosomally synthesized and post-translationally modified peptides, polyphenols, polysaccharides (e.g., chitosan), and biopolymers. For an in-depth review of secondary metabolites see, for example, Vining, Annu. Rev. Microbiol. 44:395-427, 1990.

Secondary metabolites useful for compositions and methods described herein include those that alter a natural function of an endosymbiont (e.g., primary or secondary endosymbiont), bacteriocyte, or extracellular symbiont. In some instances, one or more secondary metabolites described herein is isolated from a high throughput screening (HTS) for antimicrobial compounds. For example, a HTS screen identified 49 antibacterial extracts that have specificity against gram positive and gram negative bacteria from over 39,000 crude extracts from organisms growing in diverse ecosystems of one specific region. In some instances, the secondary metabolite is transported inside a bacteriocyte.

In some instances, the small molecule is an inhibitor of vitamin synthesis. In some instances, the vitamin synthesis inhibitor is a vitamin precursor analog. In certain instances, the vitamin precursor analog is pantothenol. For example, pantothenol may be used to inhibit vitamin B5 synthesis in Buchnera in aphids.

In some instances, the small molecule is an amino acid analog. In certain instances, the amino acid analog is L-canvanine, D-arginine, D-valine, D-methionine, D-phenylalanine, D-histidine, D-tryptophan, D-threonine, D-leucine, L-NG-nitroarginine, or a combination thereof.

In some instances the small molecule is a natural antimicrobial compound, such as propionic acid, levulinic acid, trans-cinnemaldehdye, nisin, or low molecular weight chitosan. The secondary metabolite described herein may be formulated in a composition for any of the uses described herein. The compositions disclosed herein may include any number or type (e.g., classes) of secondary metabolites, such as at least about any one of 1 secondary metabolite, 2, 3, 4, 5, 10, 15, 20, or more secondary metabolites. A suitable concentration of each secondary metabolite in the composition depends on factors such as efficacy, stability of the secondary metabolite, number of distinct secondary metabolites, the formulation, and methods of application of the composition. In some instances, wherein the composition includes at least two types of secondary metabolites, the concentration of each type of secondary metabolite may be the same or different.

A modulating agent including a secondary metabolite as described herein can be contacted with the target host in an amount and for a time sufficient to: (a) reach a target level (e.g., a predetermined or threshold level) of secondary metabolite concentration inside a target host; (b) reach a target level (e.g., a predetermined or threshold level) of secondary metabolite concentration inside a target host gut; (c) reach a target level (e.g., a predetermined or threshold level) of secondary metabolite concentration inside a target host bacteriocyte; (d) modulate the level, or an activity, of one or more microorganism (e.g., endosymbiont) in the target host; or/and (e) modulate fitness of the target host.

As illustrated by Example 15, secondary metabolites (e.g., gossypol) can be used as modulating agents that target an endosymbiotic bacterium, such as a Buchnera spp., in an insect host, such as aphids, to decrease the fitness of the host (e.g., as outlined herein). As further illustrated by Examples 8-10, 12-14, 16, 20, and 21, small molecules, such as trans-cinnemaldehyde, levulinic acid, chitosan, vitamin analogs, or amino acid transport inhibitors, can be used as modulating agents that target an endosymbiotic bacterium, such as a Buchnera spp., in an insect host, such as aphids, to decrease the fitness of the host (e.g., as outlined herein).

iv. Bacteria as Modulating Agents

In some instances, the modulating agent described herein includes one or more bacteria. Numerous bacteria are useful in the compositions and methods described herein. In some instances, the agent is a bacterial species endogenously found in the host. In some instances, the bacterial modulating agent is an endosymbiotic bacterial species. In some instances, the bacterial modulating agent is a pathogen in the host. Non-limiting examples of bacteria that may be used as modulating agents include all bacterial species described herein in Section II of the detailed description and those listed in Table 1. For example, the modulating agent may be a bacterial species from any bacterial phyla present in insect guts, including Gammaproteobacteria, Alphaproteobacteria, Betaproteobacteria, Bacteroidetes, Firmicutes (e.g., Lactobacillus and Bacillus spp.), Clostridia, Actinomycetes, Spirochetes, Verrucomicrobia, and Actinobacteria.

In some instances, the modulating agent is a bacterium that disrupts microbial diversity or otherwise alters the microbiota of the host in a manner detrimental to the host. In one instance, bacteria may be provided to disrupt the microbiota of insects. For example, the bacterial modulating agent may compete with, displace, and/or reduce a population of symbiotic bacteria in an insect. For example, a bacterium may decrease the fitness of the host by competing with symbiotic bacteria in the host that confer resistance to a pesticide (e.g., a pesticide listed in Table 12). In other instances, a bacterium may be a pathogen that decreases the fitness of the host by causing disease in the host.

The bacterial modulating agents discussed herein can be used to alter the level, activity, or metabolism of target microorganisms as indicated in the sections for decreasing the fitness of insects, such as, aphids.

v. Modifications to Modulating Agents

(a) Fusions

Any of the modulating agents described herein may be fused or linked to an additional moiety. In some instances, the modulating agent includes a fusion of one or more additional moieties (e.g., 1 additional moiety, 2, 3, 4, 5, 6, 7, 8, 9, 10, or more additional moieties). In some instances, the additional moiety is any one of the modulating agents described herein (e.g., a peptide, polypeptide, small molecule, or antibiotic). Alternatively, the additional moiety may not act as modulating agent itself but may instead serve a secondary function. For example, the additional moiety may to help the modulating agent access, bind, or become activated at a target site in the host (e.g., at a host gut or a host bacteriocyte) or at a target microorganism resident in the host (e.g., aphid).

In some instances, the additional moiety may help the modulating agent penetrate a target host cell or target microorganism resident in the host. For example, the additional moiety may include a cell penetrating peptide. Cell penetrating peptides (CPPs) may be natural sequences derived from proteins; chimeric peptides that are formed by the fusion of two natural sequences; or synthetic CPPs, which are synthetically designed sequences based on structure-activity studies. In some instances, CPPs have the capacity to ubiquitously cross cellular membranes (e.g., prokaryotic and eukaryotic cellular membranes) with limited toxicity. Further, CPPs may have the capacity to cross cellular membranes via energy-dependent and/or independent mechanisms, without the necessity of a chiral recognition by specific receptors. CPPs can be bound to any of the modulating agents described herein. For example, a CPP can be bound to an antimicrobial peptide (AMP), e.g., a scorpion peptide, e.g., UY192 fused to a cell penetrating peptide (e.g., YGRKKRRQRRRFLSTIWNGIKGLLFAM; SEQ ID NO: 237). Non-limiting examples of CPPs are listed in Table 11.

TABLE 11
Examples of Cell Penetrating Peptides (CPPs)

| Peptide | Origin | Sequence |
|---|---|---|
| Protein-derived | | |
| Penetratin | Antennapedia | RQIKIWFQNRRMKWKK |
| | | (SEQ ID NO: 213) |
| Tat peptide | Tat | GRKKRRQRRRPPQ |
| | | (SEQ ID NO: 214) |
| pVEC | Cadherin | LLIILRRRIRKQAHAHSK |
| | | (SEQ ID NO: 215) |
| Chimeric | | |
| Transportan | Galanine/Mastoparan | GWTLNSAGYLLGKINLKALAALAKKIL |
| | | (SEQ ID NO: 216) |
| MPG | HIV-gp41/SV40 | GALFLGFLGAAGSTMGAWSQPKKKRKV |
| | T-antigen | (SEQ ID NO: 217) |
| Pep-1 | HIV-reverse | KETWWETVVWTEWSQPKKKRKV |
| | transcriptase/SV40 T- | (SEQ ID NO: 218) |
| | antigen | |
| Synthetic | | |
| Polyarginines | Based on Tat peptide | (R)ₙ; 6 < n < 12 |
| MAP | de novo | KLALKLALKALKAALKLA |
| | | (SEQ ID NO: 219) |
| R₆W₃ | Based on penetratin | RRWWRRWRR |
| | | (SEQ ID NO: 220) |

In other instances, the additional moiety helps the modulating agent bind a target microorganism (e.g., a fungi or bacterium) resident in the host. The additional moiety may include one or more targeting domains. In some instances, the targeting domain may target the modulating agent to one or more microorganisms (e.g., bacterium or fungus) resident in the gut of the host. In some instances, the targeting domain may target the modulating agent to a specific region of the host (e.g., host gut or bacteriocyte) to access microorganisms that are generally present in said region of the host. For example, the targeting domain may target the modulating agent to the foregut, midgut, or hindgut of the host. In other instances, the targeting domain may target the modulating agent to a bacteriocyte in the host and/or one or more specific bacteria resident in a host bacteriocyte. For example, the targeting domain may be Galanthus nivalis lectin or agglutinin (GNA) bound to a modulating agent described herein, e.g., an AMP, e.g., a scorpion peptide, e.g., Uy192.

(b) Pre- or Pro-Domains

In some instances, the modulating agent may include a pre- or pro-amino acid sequence. For example, the modulating agent may be an inactive protein or peptide that can be activated by cleavage or post-translational modification of a pre- or pro-sequence. In some instances, the modulating agent is engineered with an inactivating pre- or pro-sequence. For example, the pre- or pro-sequence may obscure an activation site on the modulating agent, e.g., a receptor binding site, or may induce a conformational change in the modulating agent. Thus, upon cleavage of the pre- or pro-sequence, the modulating agent is activated.

Alternatively, the modulating agent may include a pre- or pro-small molecule, e.g., an antibiotic. The modulating agent may be an inactive small molecule described herein that can be activated in a target environment inside the host. For example, the small molecule may be activated upon reaching a certain pH in the host gut.

(c) Linkers

In instances where the modulating agent is connected to an additional moiety, the modulating agent may further include a linker. For example, the linker may be a chemical bond, e.g., one or more covalent bonds or non-covalent bonds. In some instances, the linker may be a peptide linker (e.g., 2, 3, 4, 5, 6, 8, 10, 12, 14, 16, 20, 25, 30, 35, 40, or more amino acids longer). The linker maybe include any flexible, rigid, or cleavable linkers described herein.

A flexible peptide linker may include any of those commonly used in the art, including linkers having sequences having primarily Gly and Ser residues (“GS” linker). Flexible linkers may be useful for joining domains that require a certain degree of movement or interaction and may include small, non-polar (e.g. Gly) or polar (e.g. Ser or Thr) amino acids.

Alternatively, a peptide linker may be a rigid linker. Rigid linkers are useful to keep a fixed distance between moieties and to maintain their independent functions. Rigid linkers may also be useful when a spatial separation of the domains is critical to preserve the stability or bioactivity of one or more components in the fusion. Rigid linkers may, for example, have an alpha helix-structure or Pro-rich sequence, (XP)ₙ, with X designating any amino acid, preferably Ala, Lys, or Glu.

In yet other instances, a peptide linker may be a cleavable linker. In some instances, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between two Cys residues. In vitro thrombin treatment of CPRSC results in the cleavage of the thrombin-sensitive sequence, while the reversible disulfide linkage remains intact. Such linkers are known and described, e.g., in Chen et al., Adv. Drug Deliv. Rev. 65(10):1357-1369, 2013. Cleavage of linkers in fusions may also be carried out by proteases that are expressed in vivo under conditions in specific cells or tissues of the host or microorganisms resident in the host. In some instances, cleavage of the linker may release a free functional, modulating agent upon reaching a target site or cell.

Fusions described herein may alternatively be linked by a linking molecule, including a hydrophobic linker, such as a negatively charged sulfonate group; lipids, such as a poly (—CH2-) hydrocarbon chains, such as polyethylene glycol (PEG) group, unsaturated variants thereof, hydroxylated variants thereof, amidated or otherwise N-containing variants thereof, non-carbon linkers; carbohydrate linkers; phosphodiester linkers, or other molecule capable of covalently linking two or more molecules, e.g., two modulating agents. Non-covalent linkers may be used, such as hydrophobic lipid globules to which the modulating agent is linked, for example, through a hydrophobic region of the modulating agent or a hydrophobic extension of the modulating agent, such as a series of residues rich in leucine, isoleucine, valine, or perhaps also alanine, phenylalanine, or even tyrosine, methionine, glycine or other hydrophobic residue. The modulating agent may be linked using charge-based chemistry, such that a positively charged moiety of the modulating agent is linked to a negative charge of another modulating agent or an additional moiety.

### IV. Formulations and Compositions

The compositions described herein may be formulated either in pure form (e.g., the composition contains only the modulating agent) or together with one or more additional agents (such as excipient, delivery vehicle, carrier, diluent, stabilizer, etc.) to facilitate application or delivery of the compositions. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil.

In some instances, the composition includes a delivery vehicle or carrier. In some instances, the delivery vehicle includes an excipient. Exemplary excipients include, but are not limited to, solid or liquid carrier materials, solvents, stabilizers, slow-release excipients, colorings, and surface-active substances (surfactants). In some instances, the delivery vehicle is a stabilizing vehicle. In some instances, the stabilizing vehicle includes a stabilizing excipient. Exemplary stabilizing excipients include, but are not limited to, epoxidized vegetable oils, antifoaming agents, e.g. silicone oil, preservatives, viscosity regulators, binding agents and tackifiers. In some instances, the stabilizing vehicle is a buffer suitable for the modulating agent. In some instances, the composition is microencapsulated in a polymer bead delivery vehicle. In some instances, the stabilizing vehicle protects the modulating agent against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

Depending on the intended objectives and prevailing circumstances, the composition may be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can. In some instances, the composition is present in the waste (such as feces) of the pest. In some instances, the composition is present in or on a live pest.

In some instances, the delivery vehicle is the food or water of the host. In other instances, the delivery vehicle is a food source for the host. In some instances, the delivery vehicle is a food bait for the host. In some instances, the composition is a comestible agent consumed by the host. In some instances, the composition is delivered by the host to a second host, and consumed by the second host. In some instances, the composition is consumed by the host or a second host, and the composition is released to the surrounding of the host or the second host via the waste (such as feces) of the host or the second host. In some instances, the modulating agent is included in food bait intended to be consumed by a host or carried back to its colony.

In some instances, the modulating agent may make up about 0.1% to about 100% of the composition, such as any one of about 0.01% to about 100%, about 1% to about 99.9%, about 0.1% to about 10%, about 1% to about 25%, about 10% to about 50%, about 50% to about 99%, or about 0.1% to about 90% of active ingredients (such as phage, lysin or bacteriocin). In some instances, the composition includes at least any of 0.1%, 0.5%, 1%, 5%, 10%, 20%, 30%, 40%, 50%, 60%, 70%, 80%, 90%, 95%, or more active ingredients (such as phage, lysin or bacteriocin). In some instances, the concentrated agents are preferred as commercial products, the final user normally uses diluted agents, which have a substantially lower concentration of active ingredient.

Any of the formulations described herein may be used in the form of a bait, a coil, an electric mat, a smoking preparation, a fumigant, or a sheet.

i. Liquid Formulations

The compositions provided herein may be in a liquid formulation. Liquid formulations are generally mixed with water, but in some instances may be used with crop oil, diesel fuel, kerosene or other light oil as a carrier. The amount of active ingredient often ranges from about 0.5 to about 80 percent by weight.

An emulsifiable concentrate formulation may contain a liquid active ingredient, one or more petroleum-based solvents, and an agent that allows the formulation to be mixed with water to form an emulsion. Such concentrates may be used in agricultural, ornamental and turf, forestry, structural, food processing, livestock, and public health pest formulations. These may be adaptable to application equipment from small portable sprayers to hydraulic sprayers, low-volume ground sprayers, mist blowers, and low-volume aircraft sprayers. Some active ingredients are readily dissolve in a liquid carrier. When mixed with a carrier, they form a solution that does not settle out or separate, e.g., a homogenous solution. Formulations of these types may include an active ingredient, a carrier, and one or more other ingredients. Solutions may be used in any type of sprayer, indoors and outdoors.

In some instances, the composition may be formulated as an invert emulsion. An invert emulsion is a water-soluble active ingredient dispersed in an oil carrier. Invert emulsions require an emulsifier that allows the active ingredient to be mixed with a large volume of petroleum-based carrier, usually fuel oil. Invert emulsions aid in reducing drift. With other formulations, some spray drift results when water droplets begin to evaporate before reaching target surfaces; as a result the droplets become very small and lightweight. Because oil evaporates more slowly than water, invert emulsion droplets shrink less and more active ingredient reaches the target. Oil further helps to reduce runoff and improve rain resistance. It further serves as a sticker-spreader by improving surface coverage and absorption. Because droplets are relatively large and heavy, it is difficult to get thorough coverage on the undersides of foliage. Invert emulsions are most commonly used along rights-of-way where drift to susceptible non-target areas can be a problem.

A flowable or liquid formulation combines many of the characteristics of emulsifiable concentrates and wettable powders. Manufacturers use these formulations when the active ingredient is a solid that does not dissolve in either water or oil. The active ingredient, impregnated on a substance such as clay, is ground to a very fine powder. The powder is then suspended in a small amount of liquid. The resulting liquid product is quite thick. Flowables and liquids share many of the features of emulsifiable concentrates, and they have similar disadvantages. They require moderate agitation to keep them in suspension and leave visible residues, similar to those of wettable powders.

Flowables/liquids are easy to handle and apply. Because they are liquids, they are subject to spilling and splashing. They contain solid particles, so they contribute to abrasive wear of nozzles and pumps. Flowable and liquid suspensions settle out in their containers. Because flowable and liquid formulations tend to settle, packaging in containers of five gallons or less makes remixing easier.

Aerosol formulations contain one or more active ingredients and a solvent. Most aerosols contain a low percentage of active ingredients. There are two types of aerosol formulations—the ready-to-use type commonly available in pressurized sealed containers and those products used in electrical or gasoline-powered aerosol generators that release the formulation as a smoke or fog.

Ready to use aerosol formulations are usually small, self-contained units that release the formulation when the nozzle valve is triggered. The formulation is driven through a fine opening by an inert gas under pressure, creating fine droplets. These products are used in greenhouses, in small areas inside buildings, or in localized outdoor areas. Commercial models, which hold five to 5 pounds of active ingredient, are usually refillable.

Smoke or fog aerosol formulations are not under pressure. They are used in machines that break the liquid formulation into a fine mist or fog (aerosol) using a rapidly whirling disk or heated surface.

ii. Dry or Solid Formulations

Dry formulations can be divided into two types: ready-to-use and concentrates that must be mixed with water to be applied as a spray. Most dust formulations are ready to use and contain a low percentage of active ingredients (less than about 10 percent by weight), plus a very fine, dry inert carrier made from talc, chalk, clay, nut hulls, or volcanic ash. The size of individual dust particles varies. A few dust formulations are concentrates and contain a high percentage of active ingredients. Mix these with dry inert carriers before applying. Dusts are always used dry and can easily drift to non-target sites.

iii. Granule or Pellet Formulations

In some instances, the composition is formulated as granules. Granular formulations are similar to dust formulations, except granular particles are larger and heavier. The coarse particles may be made from materials such as clay, corncobs, or walnut shells. The active ingredient either coats the outside of the granules or is absorbed into them. The amount of active ingredient may be relatively low, usually ranging from about 0.5 to about 15 percent by weight. Granular formulations are most often used to apply to the soil, insects living in the soil, or absorption into plants through the roots. Granular formulations are sometimes applied by airplane or helicopter to minimize drift or to penetrate dense vegetation. Once applied, granules may release the active ingredient slowly. Some granules require soil moisture to release the active ingredient. Granular formulations also are used to control larval mosquitoes and other aquatic pests. Granules are used in agricultural, structural, ornamental, turf, aquatic, right-of-way, and public health (biting insect) pest-control operations.

In some instances, the composition is formulated as pellets. Most pellet formulations are very similar to granular formulations; the terms are used interchangeably. In a pellet formulation, however, all the particles are the same weight and shape. The uniformity of the particles allows use with precision application equipment.

iv. Powders

In some instances, the composition is formulated as a powder. In some instances, the composition is formulated as a wettable powder. Wettable powders are dry, finely ground formulations that look like dusts. They usually must be mixed with water for application as a spray. A few products, however, may be applied either as a dust or as a wettable powder—the choice is left to the applicator. Wettable powders have about 1 to about 95 percent active ingredient by weight; in some cases more than about 50 percent. The particles do not dissolve in water. They settle out quickly unless constantly agitated to keep them suspended. They can be used for most pest problems and in most types of spray equipment where agitation is possible. Wettable powders have excellent residual activity. Because of their physical properties, most of the formulation remains on the surface of treated porous materials such as concrete, plaster, and untreated wood. In such cases, only the water penetrates the material.

In some instances, the composition is formulated as a soluble powder. Soluble powder formulations look like wettable powders. However, when mixed with water, soluble powders dissolve readily and form a true solution. After they are mixed thoroughly, no additional agitation is necessary. The amount of active ingredient in soluble powders ranges from about 15 to about 95 percent by weight; in some cases more than about 50 percent. Soluble powders have all the advantages of wettable powders and none of the disadvantages, except the inhalation hazard during mixing.

In some instances, the composition is formulated as a water-dispersible granule. Water-dispersible granules, also known as dry flowables, are like wettable powders, except instead of being dust-like, they are formulated as small, easily measured granules. Water-dispersible granules must be mixed with water to be applied. Once in water, the granules break apart into fine particles similar to wettable powders. The formulation requires constant agitation to keep it suspended in water. The percentage of active ingredient is high, often as much as 90 percent by weight. Water-dispersible granules share many of the same advantages and disadvantages of wettable powders, except they are more easily measured and mixed. Because of low dust, they cause less inhalation hazard to the applicator during handling

v. Bait

In some instances, the composition includes a bait. The bait can be in any suitable form, such as a solid, paste, pellet or powdered form. The bait can also be carried away by the host back to a population of said host (e.g., a colony or hive). The bait can then act as a food source for other members of the colony, thus providing an effective modulating agent for a large number of hosts and potentially an entire host colony.

The baits can be provided in a suitable “housing” or “trap.” Such housings and traps are commercially available and existing traps can be adapted to include the compositions described herein. The housing or trap can be box-shaped for example, and can be provided in pre-formed condition or can be formed of foldable cardboard for example. Suitable materials for a housing or trap include plastics and cardboard, particularly corrugated cardboard. The inside surfaces of the traps can be lined with a sticky substance in order to restrict movement of the host once inside the trap. The housing or trap can contain a suitable trough inside which can hold the bait in place. A trap is distinguished from a housing because the host cannot readily leave a trap following entry, whereas a housing acts as a “feeding station” which provides the host with a preferred environment in which they can feed and feel safe from predators.

vi. Attractants

In some instances, the composition includes an attractant (e.g., a chemoattractant). The attractant may attract an adult host or immature host (e.g., larva) to the vicinity of the composition. Attractants include pheromones, a chemical that is secreted by an animal, especially an insect, which influences the behavior or development of others of the same species. Other attractants include sugar and protein hydrolysate syrups, yeasts, and rotting meat. Attractants also can be combined with an active ingredient and sprayed onto foliage or other items in the treatment area.

Various attractants are known which influence host behavior as a host's search for food, oviposition or mating sites, or mates. Attractants useful in the methods and compositions described herein include, for example, eugenol, phenethyl propionate, ethyl dimethylisobutyl-cyclopropane carboxylate, propyl benszodioxancarboxylate, cis-7,8-epoxy-2-methyloctadecane, trans-8,trans-0-dodecadienol, cis-9-tetradecenal (with cis-11-hexadecenal), trans-11-tetradecenal, cis-11-hexadecenal, (Z)-11,12-hexadecadienal, cis-7-dodecenyl acetate, cis-8-dodecenyul acetate, cis-9-dodecenyl acetate, cis-9-tetradecenyl acetate, cis-11-tetradecenyl acetate, trans-11-tetradecenyl acetate (with cis-11), cis-9,trans-11-tetradecadienyl acetate (with cis-9,trans-12), cis-9,trans-12-tetradecadienyl acetate, cis-7,cis-11-hexadecadienyl acetate (with cis-7,trans-11), cis-3,cis-13-octadecadienyl acetate, trans-3,cis-13-octadecadienyl acetate, anethole and isoamyl salicylate.

Means other than chemoattractants may also be used to attract insects, including lights in various wavelengths or colors.

vii. Nanocapsules/Microencapsulation/Liposomes

In some instances, the composition is provided in a microencapsulated formulation. Microencapsulated formulations are mixed with water and sprayed in the same manner as other sprayable formulations. After spraying, the plastic coating breaks down and slowly releases the active ingredient.

viii. Carriers

Any of the compositions described herein may be formulated to include the modulating agent described herein and an inert carrier. Such carrier can be a solid carrier, a liquid carrier, a gel carrier, and/or a gaseous carrier. In certain instances, the carrier can be a seed coating. The seed coating is any non-naturally occurring formulation that adheres, in whole or part, to the surface of the seed. The formulation may further include an adjuvant or surfactant. The formulation can also include one or more modulating agents to enlarge the action spectrum.

A solid carrier used for formulation includes finely-divided powder or granules of clay (e.g. kaolin clay, diatomaceous earth, bentonite, Fubasami clay, acid clay, etc.), synthetic hydrated silicon oxide, talc, ceramics, other inorganic minerals (e.g., sericite, quartz, sulfur, activated carbon, calcium carbonate, hydrated silica, etc.), a substance which can be sublimated and is in the solid form at room temperature (e.g., 2,4,6-triisopropyl-1,3,5-trioxane, naphthalene, p-dichlorobenzene, camphor, adamantan, etc.); wool; silk; cotton; hemp; pulp; synthetic resins (e.g., polyethylene resins such as low-density polyethylene, straight low-density polyethylene and high-density polyethylene; ethylene-vinyl ester copolymers such as ethylene-vinyl acetate copolymers; ethylene-methacrylic acid ester copolymers such as ethylene-methyl methacrylate copolymers and ethylene-ethyl methacrylate copolymers; ethylene-acrylic acid ester copolymers such as ethylene-methyl acrylate copolymers and ethylene-ethyl acrylate copolymers; ethylene-vinylcarboxylic acid copolymers such as ethylene-acrylic acid copolymers; ethylene-tetracyclododecene copolymers; polypropylene resins such as propylene homopolymers and propylene-ethylene copolymers; poly-4-methylpentene-1, polybutene-1, polybutadiene, polystyrene; acrylonitrile-styrene resins; styrene elastomers such as acrylonitrile-butadiene-styrene resins, styrene-conjugated diene block copolymers, and styrene-conjugated diene block copolymer hydrides; fluororesins; acrylic resins such as poly(methyl methacrylate); polyamide resins such as nylon 6 and nylon 66; polyester resins such as polyethylene terephthalate, polyethylene naphthalate, polybutylene terephthalate, and polycyclohexylenedimethylene terephthalate; polycarbonates, polyacetals, polyacrylsulfones, polyarylates, hydroxybenzoic acid polyesters, polyetherimides, polyester carbonates, polyphenylene ether resins, polyvinyl chloride, polyvinylidene chloride, polyurethane, and porous resins such as foamed polyurethane, foamed polypropylene, or foamed ethylene, etc.), glasses, metals, ceramics, fibers, cloths, knitted fabrics, sheets, papers, yarn, foam, porous substances, and multifilaments.

A liquid carrier may include, for example, aromatic or aliphatic hydrocarbons (e.g., xylene, toluene, alkylnaphthalene, phenylxylylethane, kerosine, gas oil, hexane, cyclohexane, etc.), halogenated hydrocarbons (e.g., chlorobenzene, dichloromethane, dichloroethane, trichloroethane, etc.), alcohols (e.g., methanol, ethanol, isopropyl alcohol, butanol, hexanol, benzyl alcohol, ethylene glycol, etc.), ethers (e.g., diethyl ether, ethylene glycol dimethyl ether, diethylene glycol monomethyl ether, diethylene glycol monoethyl ether, propylene glycol monomethyl ether, tetrahydrofuran, dioxane, etc.), esters (e.g., ethyl acetate, butyl acetate, etc.), ketones (e.g., acetone, methyl ethyl ketone, methyl isobutyl ketone, cyclohexanone, etc.), nitriles (e.g., acetonitrile, isobutyronitrile, etc.), sulfoxides (e.g., dimethyl sulfoxide, etc.), amides (e.g., N,N-dimethylformamide, N,N-dimethylacetamide, cyclic imides (e.g. N-methylpyrrolidone) alkylidene carbonates (e.g., propylene carbonate, etc.), vegetable oil (e.g., soybean oil, cottonseed oil, etc.), vegetable essential oils (e.g., orange oil, hyssop oil, lemon oil, etc.), or water.

A gaseous carrier may include, for example, butane gas, flon gas, liquefied petroleum gas (LPG), dimethyl ether, and carbon dioxide gas.

ix. Adjuvants

In some instances, the composition provided herein may include an adjuvant. Adjuvants are chemicals that do not possess activity. Adjuvants are either pre-mixed in the formulation or added to the spray tank to improve mixing or application or to enhance performance. They are used extensively in products designed for foliar applications. Adjuvants can be used to customize the formulation to specific needs and compensate for local conditions. Adjuvants may be designed to perform specific functions, including wetting, spreading, sticking, reducing evaporation, reducing volatilization, buffering, emulsifying, dispersing, reducing spray drift, and reducing foaming. No single adjuvant can perform all these functions, but compatible adjuvants often can be combined to perform multiple functions simultaneously.

Among nonlimiting examples of adjuvants included in the formulation are binders, dispersants and stabilizers, specifically, for example, casein, gelatin, polysaccharides (e.g., starch, gum arabic, cellulose derivatives, alginic acid, etc.), lignin derivatives, bentonite, sugars, synthetic water-soluble polymers (e.g., polyvinyl alcohol, polyvinylpyrrolidone, polyacrylic acid, etc.), PAP (acidic isopropyl phosphate), BHT (2,6-di-t-butyl-4-methylphenol), BHA (a mixture of 2-t-butyl-4-methoxyphenol and 3-t-butyl-4-methoxyphenol), vegetable oils, mineral oils, fatty acids and fatty acid esters.

x. Surfactants

In some instances, the composition provided herein includes a surfactant. Surfactants, also called wetting agents and spreaders, physically alter the surface tension of a spray droplet. For a formulation to perform its function properly, a spray droplet must be able to wet the foliage and spread out evenly over a leaf. Surfactants enlarge the area of formulation coverage, thereby increasing the pest's exposure to the chemical. Surfactants are particularly important when applying a formulation to waxy or hairy leaves. Without proper wetting and spreading, spray droplets often run off or fail to cover leaf surfaces adequately. Too much surfactant, however, can cause excessive runoff and reduce efficacy.

Surfactants are classified by the way they ionize or split apart into electrically charged atoms or molecules called ions. A surfactant with a negative charge is anionic. One with a positive charge is cationic, and one with no electrical charge is nonionic. Formulation activity in the presence of a nonionic surfactant can be quite different from activity in the presence of a cationic or anionic surfactant. Selecting the wrong surfactant can reduce the efficacy of a pesticide product and injure the target plant. Anionic surfactants are most effective when used with contact pesticides (pesticides that control the pest by direct contact rather than being absorbed systemically). Cationic surfactants should never be used as stand-alone surfactants because they usually are phytotoxic.

Nonionic surfactants, often used with systemic pesticides, help pesticide sprays penetrate plant cuticles. Nonionic surfactants are compatible with most pesticides, and most EPA-registered pesticides that require a surfactant recommend a nonionic type. Adjuvants include, but are not limited to, stickers, extenders, plant penetrants, compatibility agents, buffers or pH modifiers, drift control additives, defoaming agents, and thickeners.

Among nonlimiting examples of surfactants included in the compositions described herein are alkyl sulfate ester salts, alkyl sulfonates, alkyl aryl sulfonates, alkyl aryl ethers and polyoxyethylenated products thereof, polyethylene glycol ethers, polyvalent alcohol esters and sugar alcohol derivatives.

xi. Combinations

In formulations and in the use forms prepared from these formulations, the modulating agent may be in a mixture with other active compounds, such as pesticidal agents (e.g., insecticides, sterilants, acaricides, nematicides, molluscicides, or fungicides; see, e.g., pesticides listed in Table 12), attractants, growth-regulating substances, or herbicides. As used herein, the term “pesticidal agent” refers to any substance or mixture of substances intended for preventing, destroying, repelling, or mitigating any pest. A pesticide can be a chemical substance or biological agent used against pests including insects, pathogens, weeds, and microbes that compete with humans for food, destroy property, spread disease, or are a nuisance. The term “pesticidal agent” may further encompass other bioactive molecules such as antibiotics, antivirals pesticides, antifungals, antihelminthics, nutrients, pollen, sucrose, and/or agents that stun or slow insect movement.

In instances where the modulating agent is applied to plants, a mixture with other known compounds, such as herbicides, fertilizers, growth regulators, safeners, semiochemicals, or else with agents for improving plant properties is also possible.

### V. Delivery

A host described herein can be exposed to any of the compositions described herein in any suitable manner that permits delivering or administering the composition to the insect. The modulating agent may be delivered either alone or in combination with other active or inactive substances and may be applied by, for example, spraying, microinjection, through plants, pouring, dipping, in the form of concentrated liquids, gels, solutions, suspensions, sprays, powders, pellets, briquettes, bricks and the like, formulated to deliver an effective concentration of the modulating agent. Amounts and locations for application of the compositions described herein are generally determined by the habits of the host, the lifecycle stage at which the microorganisms of the host can be targeted by the modulating agent, the site where the application is to be made, and the physical and functional characteristics of the modulating agent. The modulating agents described herein may be administered to the insect by oral ingestion, but may also be administered by means which permit penetration through the cuticle or penetration of the insect respiratory system.

In some instances, the insect can be simply “soaked” or “sprayed” with a solution including the modulating agent. Alternatively, the modulating agent can be linked to a food component (e.g., comestible) of the insect for ease of delivery and/or in order to increase uptake of the modulating agent by the insect. Methods for oral introduction include, for example, directly mixing a modulating agent with the insect's food, spraying the modulating agent in the insect's habitat or field, as well as engineered approaches in which a species that is used as food is engineered to express a modulating agent, then fed to the insect to be affected. In some instances, for example, the modulating agent composition can be incorporated into, or overlaid on the top of, the insect's diet. For example, the modulating agent composition can be sprayed onto a field of crops which an insect inhabits.

In some instances, the composition is sprayed directly onto a plant e.g., crops, by e.g., backpack spraying, aerial spraying, crop spraying/dusting etc. In instances where the modulating agent is delivered to a plant, the plant receiving the modulating agent may be at any stage of plant growth. For example, formulated modulating agents can be applied as a seed-coating or root treatment in early stages of plant growth or as a total plant treatment at later stages of the crop cycle. In some instances, the modulating agent may be applied as a topical agent to a plant, such that the host insect ingests or otherwise comes in contact with the plant upon interacting with the plant.

Further, the modulating agent may be applied (e.g., in the soil in which a plant grows, or in the water that is used to water the plant) as a systemic agent that is absorbed and distributed through the tissues (e.g., stems or leafs) of a plant or animal host, such that an insect feeding thereon will obtain an effective dose of the modulating agent. In some instances, plants or food organisms may be genetically transformed to express the modulating agent such that a host feeding upon the plant or food organism will ingest the modulating agent.

Delayed or continuous release can also be accomplished by coating the modulating agent or a composition with the modulating agent(s) with a dissolvable or bioerodable coating layer, such as gelatin, which coating dissolves or erodes in the environment of use, to then make the modulating agent available, or by dispersing the agent in a dissolvable or erodable matrix. Such continuous release and/or dispensing means devices may be advantageously employed to consistently maintain an effective concentration of one or more of the modulating agents described herein in a specific host habitat.

The modulating agent can also be incorporated into the medium in which the insect grows, lives, reproduces, feeds, or infests. For example, a modulating agent can be incorporated into a food container, feeding station, protective wrapping, or a hive. For some applications the modulating agent may be bound to a solid support for application in powder form or in a “trap” or “feeding station.” As an example, for applications where the composition is to be used in a trap or as bait for a particular host insect, the compositions may also be bound to a solid support or encapsulated in a time-release material. For example, the compositions described herein can be administered by delivering the composition to at least one habitat where an agricultural pest (e.g., aphid) grows, lives, reproduces, or feeds.

### VI. Screening

Included herein are methods for screening for modulating agents that are effective to alter the microbiota of a host (e.g., insect) and thereby decrease host fitness. The screening assays provided herein may be effective to identify one or more modulating agents (e.g., phage) that target symbiotic microorganisms resident in the host and thereby decrease the fitness of the host. For example, the identified modulating agent (e.g., phage) may be effective to decrease the viability of pesticide- or allelochemical-degrading microorganisms (e.g., bacteria, e.g., a bacterium that degrades a pesticide listed in Table 12), thereby increasing the hosts sensitivity to a pesticide (e.g., sensitivity to a pesticide listed in Table 12) or allelochemical agent.

For example, a phage library may be screened to identify a phage that targets a specific endosymbiotic microorganism resident in a host. In some instances, the phage library may be provided in the form of one or more environmental samples (e.g., soil, pond sediments, or sewage water). Alternatively, the phage library may be generated from laboratory isolates. The phage library may be co-cultured with a target bacterial strain. After incubation with the bacterial strain, phage that successfully infect and lyse the target bacteria are enriched in the culture media. The phage-enriched culture may be sub-cultured with additional bacteria any number of times to further enrich for phage of interest. The phage may be isolated for use as a modulating agent in any of the methods or compositions described herein, wherein the phage alters the microbiota of the host in a manner that decreases host fitness.

TABLE 12
Pesticides

| | | |
|---|---|---|
| Aclonifen | Fenchlorazole-ethyl | Pendimethalin |
| Acetamiprid | Fenothiocarb | Penflufen |
| Alanycarb | Fenitrothion | Penflufen |
| Amidosulfuron | Fenpropidin | Pentachlorbenzene |
| Aminocyclopyrachlor | Fluazolate | Penthiopyrad |
| Amisulbrom | Flufenoxuron | Penthiopyrad |
| Anthraquinone | Flumetralin | Pirimiphos-methyl |
| Asulam, sodium salt | Fluxapyroxad | Prallethrin |
| Benfuracarb | Fuberidazole | Profenofos |
| Bensulide | Glufosinate-ammonium | Proquinazid |
| beta-HCH; beta-BCH | Glyphosate | Prothiofos |
| Bioresmethrin | Group: Borax, borate salts (see | Pyraclofos |
| Blasticidin-S | Group: Paraffin oils, Mineral | Pyrazachlor |
| Borax; disodium tetraborate | Halfenprox | Pyrazophos |
| Boric acid | Imiprothrin | Pyridaben |
| Bromoxynil heptanoate | Imidacloprid | Pyridalyl |
| Bromoxynil octanoate | Ipconazole | Pyridiphenthion |
| Carbosulfan | Isopyrazam | Pyrifenox |
| Chlorantraniliprole | Isopyrazam | Quinmerac |
| Chlordimeform | Lenacil | Rotenone |
| Chlorfluazuron | Magnesium phosphide | Sedaxane |
| Chlorphropham | Metaflumizone | Sedaxane |
| Climbazole | Metazachlor | Silafluofen |
| Clopyralid | Metazachlor | Sintofen |
| Copper (II) hydroxide | Metobromuron | Spinetoram |
| Cyflufenamid | Metoxuron | Sulfoxaflor |
| Cyhalothrin | Metsulfuron-methyl | Temephos |
| Cyhalothrin, gamma | Milbemectin | Thiocloprid |
| Decahydrate | Naled | Thiamethoxam |
| Diafenthiuron | Napropamide | Tolfenpyrad |
| Dimefuron | Nicosulfuron | Tralomethrin |
| Dimoxystrobin | Nitenpyram | Tributyltin compounds |
| Dinotefuran | Nitrobenzene | Tridiphane |
| Diquat dichloride | o-phenylphenol | Triflumizole |
| Dithianon | Oils | Validamycin |
| E-Phosphamidon | Oxadiargyl | Zinc phosphide |
| EPTC | Oxycarboxin | |
| Ethaboxam | Paraffin oil | |
| Ethirimol | Penconazole | |

### EXAMPLES

The following is an example of the methods of the invention. It is understood that various other embodiments may be practiced, given the general description provided above.

### Example 1: Production of a Phage Library

This Example demonstrates the acquisition of a phage collection from environmental samples.

Therapeutic Design:

Phage library collection having the following phage families: Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Gluboloviridae, Guttaviridae, Inoviridae, Leviviridae, Microviridae, Plasmaviridae, Tectiviridae

Experimental Design:

Multiple environmental samples (soil, pond sediments, sewage water) are collected in sterile 1 L flasks over a period of 2 weeks and are immediately processed as described below after collection and stored thereafter at 4° C. Solid samples are homogenized in sterile double-strength difco luria broth (LB) or tryptic soy broth (TSB) supplemented with 2 mM CaCl2) to a final volume of 100 mL. The pH and phosphate levels are measured using phosphate test strips. For purification, all samples are centrifuged at 3000-6000 g in a Megafuge 1.0R, Heraeus, or in Eppendorf centrifuge 5702 R, for 10-15 min at +4° C., and filtered through 0.2-μm low protein filters to remove all remaining bacterial cells. The supernatant is stored at 4° C. in the presence of chloroform in a glass bottle.

### Example 2: Identification of Target Specific Phage

This Example demonstrates the isolation, purification, and identification of single target specific phages from a heterogeneous phage library.

Experimental Design:

20-30 ml of the phage library described in Example 1 is diluted to a volume of 30-40 ml with LB-broth. The target bacterial strain, e.g., Buchnera, is added (50-200 μl overnight culture grown in LB-broth) to enrich phages that target this specific bacterial strain in the culture. This culture is incubated overnight at +37° C., shaken at 230 rpm. Bacteria from this enrichment culture are removed by centrifugation (3000-6000 g in Megafuge 1.0R, Heraeus, or in Eppendorf centrifuge 5702 R, 15-20 min, +4° C.) and filtered (0.2 or 0.45 μm filter). 2.5 ml of the bacteria free culture is added to 2.5 ml of LB-broth and 50-100 μl of the target bacteria to enrich the phages. The enrichment culture is grown overnight as above. A sample from this enrichment culture is centrifuged at 13,000 g for 15 min at room temperature and 10 μl of the supernatant is plated on a LB-agar containing petri dish along with 100-300 μl of the target bacteria and 3 ml of melted 0.7% soft-agar. The plates are incubated overnight at +37° C. Each of the plaques observed on the bacterial lawn are picked and transferred into 500 μl of LB-broth. A sample from this plaque-stock is further plated on the target bacteria. Plaque-purification is performed three times for all discovered phages in order to isolate a single homogenous phage from the heterogeneous phage mix.

Lysates from plates with high-titer phages (>1×10\hat{ }10 PFU/ml) are prepared by harvesting overlay plates of a host bacterium strain exhibiting confluent lysis. After being flooded with 5 ml of buffer, the soft agar overlay is macerated, clarified by centrifugation, and filter sterilized. The resulting lysates are stored at 4° C. High-titer phage lysates are further purified by isopycnic CsCl centrifugation, as described in (Summer et al., J. Bacteriol. 192:179-190, 2010).

DNA is isolated from CsCl-purified phage suspensions as described in (Summer, Methods Mol. Biol. 502:27-46, 2009). An individual isolated phage is sequenced as part of two pools of phage genomes by using a 454 pyrosequencing method. Phage genomic DNA is mixed in equimolar amounts to a final concentration of about 100 ng/L. The pooled DNA is sheared, ligated with a multiplex identifier (MID) tag specific for each of the pools, and sequenced by pyrosequencing using a full-plate reaction on a Roche FLX Titanium sequencer according to the manufacturer's protocols. The pooled phage DNA is present in two sequencing reactions. The trimmed FLX Titanium flow-gram output corresponding to each of the pools is assembled individually by using Newbler Assembler version 2.5.3 (454 Life Sciences), by adjusting the settings to include only reads containing a single MID per assembly. The identity of individual contigs is determined by PCR using primers generated against contig sequences and individual phage genomic DNA preparations as the template. Sequencher 4.8 (Gene Codes Corporation) is used for sequence assembly and editing. Phage chromosomal end structures are determined experimentally. Cohesive (cos) ends for phages are determined by sequencing off the ends of the phage genome and sequencing the PCR products derived by amplification through the ligated junction of circularized genomic DNA, as described in (Summer, Methods Mol. Biol. 502:27-46, 2009). Protein-coding regions are initially predicted using GeneMark.hmm (Lukashin et al. Nucleic Acids Res. 26:1107-1115, 1998), refined through manual analysis in Artemis (Rutherford et al., Bioinformatics 16:944-945, 2000.), and analyzed through the use of BLAST (E value cutoff of 0.005) (Camacho et al., BMC Bioinformatics 10:421, 2009). Proteins of particular interest are additionally analyzed by InterProScan (Hunter et al., Nucleic Acids Res. 40:D306-D312, 2012).

Electron microscopy of CsCl-purified phage (>1×10\hat{ }11 PFU/ml) that lysed the endosymbiotic bacteria, Buchnera, is performed by diluting stock with the tryptic soy broth buffer. Phages are applied onto thin 400-mesh carbon-coated Formvar grids, stained with 2% (wt/vol) uranyl acetate, and air dried. Specimens are observed on a JEOL 1200EX transmission electron microscope operating at an acceleration voltage of 100 kV. Five virions of each phage are measured to calculate mean values and standard deviations for dimensions of capsid and tail, where appropriate.

### Example 3: Treatment of Aphids with a Solution of Purified Phages

This Example demonstrates the ability to kill or decrease the fitness of aphids by treating them with a phage solution. This Example demonstrates that the effect of phage on aphids is mediated through the modulation of bacterial populations endogenous to the aphid that are sensitive to phages. One targeted bacterial strain is Buchnera with the phage identified in Example 2.

Aphids are one of the most important agricultural insect pests. They cause direct feeding damage to the plant and serve as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

Therapeutic Design:

Phage solutions are formulated with 0 (negative control), 10², 10⁵, or 10⁸ plaque-forming units (pfu)/ml phage from Example 2 in 10 mL of sterile water with 0.5% sucrose and essential amino acids.

Experimental Design:

To prepare for the treatment, aphids are grown in a lab environment and medium. In a climate-controlled room (16 h light photoperiod; 60±5% RH; 20±2° C.), fava bean plants are grown in a mixture of vermiculite and perlite at 24° C. with 16 h of light and 8 h of darkness. To limit maternal effects or health differences between plants, 5-10 adults from different plants are distributed among 10 two-week-old plants, and allowed to multiply to high density for 5-7 days. For experiments, second and third instar aphids are collected from healthy plants and divided into treatments so that each treatment receives approximately the same number of individuals from each of the collection plants.

Phage solutions are prepared as described herein. Wells of a 96-well plate are filled with 200 μl of artificial aphid diet (Febvay et al., Canadian Journal of Zoology 66(11):2449-2453, 1988) and the plate is covered with parafilm to make a feeding sachet. Artificial diet is either mixed with sterile water and with 0.5% sucrose and essential amino acids as a negative control or phage solutions with varying concentrations of phages. Phage solutions are mixed with artificial diet to get final concentrations of phages between 10² to 10⁸ (pfu)/ml.

For each replicate treatment, 30-50 second and third instar aphids are placed individually in wells of a 96-well plate and a feeding sachet plate is inverted above them, allowing the insects to feed through the parafilm and keeping them restricted to individual wells. Experimental aphids are kept under the same environmental conditions as aphid colonies. After the aphids are fed for 24 hr, the feeding sachet is replaced with a new one containing sterile artificial diet and a new sterile sachet is provided every 24 h for 4 days. At the time that the sachet is replaced, aphids are also checked for mortality. An aphid is counted as dead if it had turned brown or is at the bottom of the well and does not move during the observation. If an aphid is on the parafilm of the feeding sachet but not moving, it is assumed to be feeding and alive.

The status of Buchnera in aphid samples is assessed by PCR. Aphids adults from the negative control (non-phage treated) and phage treated groups are first surface-sterilized with 70% ethanol for 1 min, 10% bleach for 1 min and three washes of ultrapure water for 1 min. Total DNA is extracted from each individual (whole body) using an Insect DNA Kit (OMEGA, Bio-tek) according to the manufacturer's protocol. The primers for Buchnera, forward primer 5′-GTCGGCTCATCACATCC-3′ (SEQ ID NO: 221) and reverse primer 5′-TTCCGTCTGTATTATCTCCT-3′ (SEQ ID NO: 222), are designed based on 23S-5S rRNA sequences obtained from the Buchnera genome (Accession Number: GCA_000009605.1) (Shigenobu et al., Nature 407:81-86, 2000) using Primer 5.0 software (Primer-E Ltd., Plymouth, UK). The PCR amplification cycles included an initial denaturation step at 95° C. for 5 min, 35 cycles of 95° C. for 30 s, 55° C. for 30 s, and 72° C. for 60 s, and a final extension step of 10 min at 72° C. Amplification products from rifampicin treated and control samples are analyzed on 1% agarose gels, stained with SYBR safe, and visualized using an imaging System. Phage treated aphids show a reduction of Buchnera specific genes.

The survival rates of aphids treated with Buchnera specific phages are compared to the aphids treated with the negative control. The survival rate of aphids treated with Buchnera specific phages is decreased as compared to the control treated aphids.

### Example 4: Production of a colA Bacteriocin Solution

This Example demonstrates the production and purification of colA bacteriocin.

Construct Sequence:

(SEQ ID NO: 223)
catatgatgacccgcaccatgctgtttctggcgtgcgtggcggcgctgtat
gtgtgcattagcgcgaccgcgggcaaaccggaagaatttgcgaaactgagc
gatgaagcgccgagcaacgatcaggcgatgtatgaaagcattcagcgctat
cgccgctttgtggatggcaaccgctataacggcggccagcagcagcagcag
cagccgaaacagtgggaagtgcgcccggatctgagccgcgatcagcgcggc
aacaccaaagcgcaggtggaaattaacaaaaaaggcgataaccatgatatt
aacgcgggctggggcaaaaacattaacggcccggatagccataaagatacc
tggcatgtgggcggcagcgtgcgctggctcgag

Experimental Design:

DNA is generated by PCR with specific primers with upstream (NdeI) and downstream (XhoI) restriction sites. Forward primer GTATCTATTCCCGTCTACGAACATATGGAATTCC (SEQ ID NO: 224) and reverse primer CCGCTCGAGCCATCTGACACTTCCTCCAA (SEQ ID NO: 225). Purified PCR fragments (Nucleospin Extract II-Macherey Nagel) are digested with NdeI or XhoI and then the fragments are ligated. For colA cloning, the ligated DNA fragment is cloned into pcr2.1 (GenBank database accession number EY122872) vector (Anselme et al., BMC Biol. 6:43, 2008). The nucleotide sequence is systematically checked (Cogenics).

The plasmid with colA sequence is expressed in BL21 (DE3)/pLys. Bacteria are grown in LB broth at 30° C. At an OD600 of 0.9, isopropyl β-D-1-thiogalactopyranoside (IPTG) is added to a final concentration of 1 mM and cells were grown for 6 h. Bacteria are lysed by sonication in 100 mM NaCL, 1% Triton X-100, 100 mM Tris-base pH 9.5, and proteins are loaded onto a HisTrap HP column (GE Healthcare). The column is washed successively with 100 mM NaCl, 100 mM Tris-HCl pH 6.8, and PBS. Elution is performed with 0.3M imidazol in PBS. Desalting PD-10 columns (GE Healthcare) are used to eliminate imidazol and PBS solubilized peptides are concentrated on centrifugal filter units (Millipore).

ColA Protein sequence:

(SEQ ID NO: 211)
MTRTMLFLAC VAALYVCISA TAGKPEEFAK LSDEAPSNDQ
AMYESIQRYR RFVDGNRYNG GQQQQQQPKQ WEVRPDLSRD
QRGNTKAQVE INKKGDNHDI NAGWGKNING PDSHKDTWHV
GGSVRW

### Example 5: Treatment of Aphids with a Solution of colA Bacteriocin

This Example demonstrates the ability to kill or decrease the fitness of aphids by treating them with a bacteriocin solution. This Example demonstrates that the effect of bacteriocins on aphids is mediated through the modulation of bacterial populations endogenous to the aphid that are sensitive to ColA bacteriocin. One targeted bacterial strain is Buchnera with the bacteriocin produced in Example 1.

Aphids are one of the most important agricultural insect pests. They cause direct feeding damage to the plant and serve as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

Therapeutic Design:

ColA solutions are formulated with 0 (negative control), 0.6, 1, 50 or 100 mg/ml of ColA from Example 4 in 10 mL of sterile water with 0.5% sucrose and essential amino acids.

Experimental Design:

To prepare for the treatment, aphids are grown in a lab environment and medium. In a climate-controlled room (16 h light photoperiod; 60±5% RH; 20±2° C.), plants are grown in a mixture of vermiculite and perlite and are infested with aphids. In the same climatic conditions, E. balteatus larvae are obtained from a mass production; the hoverflies are reared with sugar, pollen and water; and the oviposition is induced by the introduction of infested host plants in the rearing cage during 3 h. The complete life cycle takes place on the host plants that are daily re-infested with aphids.

Wells of a 96-well plate are filled with 200 μl of artificial aphid diet (Febvay et al., Canadian Journal of Zoology 66(11):2449-2453, 1988) and the plate is covered with parafilm to make a feeding sachet. Artificial diet is either mixed with the solution of sterile water with 0.5% sucrose and essential amino acids as a negative control or ColA solutions with varying concentrations of ColA. ColA solutions are mixed with artificial diet to obtain final concentrations between 0.6 to 100 mg/ml.

For each replicate treatment, 30-50 second and third instar aphids are placed individually in wells of a 96-well plate and a feeding sachet plate is inverted above them, allowing the insects to feed through the parafilm and keeping them restricted to individual wells. Experimental aphids are kept under the same environmental conditions as aphid colonies. After the aphids are fed for 24 hr, the feeding sachet is replaced with a new one containing sterile artificial diet and a new sterile sachet is provided every 24 h for 4 days. At the time that the sachet is replaced, aphids are also checked for mortality. An aphid is counted as dead if it had turned brown or is at the bottom of the well and does not move during the observation. If an aphid is on the parafilm of the feeding sachet but not moving, it is assumed to be feeding and alive.

The status of Buchnera in aphid samples is assessed by PCR. Aphids adults from the negative control and phage treated are first surface-sterilized with 70% ethanol for 1 min, 10% bleach for 1 min and three washes of ultrapure water for 1 min. Total DNA is extracted from each individual (whole body) using an Insect DNA Kit (OMEGA, Bio-tek) according to the manufacturer's protocol. The primers for Buchnera, forward primer 5′-GTCGGCTCATCACATCC-3′ (SEQ ID NO: 221) and reverse primer 5′-TTCCGTCTGTATTATCTCCT-3′ (SEQ ID NO: 222), are designed based on 23S-5S rRNA sequences obtained from the Buchnera genome (Accession Number: GCA_000009605.1) (Shigenobu, et al., Nature 200.407, 81-86) using Primer 5.0 software (Primer-E Ltd., Plymouth, UK). The PCR amplification cycles included an initial denaturation step at 95° C. for 5 min, 35 cycles of 95° C. for 30 s, 55° C. for 30 s, and 72° C. for 60 s, and a final extension step of 10 min at 72° C. Amplification products from rifampicin treated and control samples are analyzed on 1% agarose gels, stained with SYBR safe, and visualized using an imaging System. ColA treated aphids show a reduction of Buchnera specific genes.

The survival rates of aphids treated with Buchnera specific ColA bacteriocin are compared to the aphids treated with the negative control. The survival rate of aphids treated with Buchnera specific ColA bacteriocin is decreased as compared to the control treated aphids.

### Example 6: Treatment of Aphids with Rifampicin Solutions

This Example demonstrates the ability to kill or decrease the fitness of aphids by treating them with rifampicin, a narrow spectrum antibiotic that inhibits DNA-dependent RNA synthesis by inhibiting a bacterial RNA polymerase. This Example demonstrates that the effect of rifampicin on aphids is mediated through the modulation of bacterial populations endogenous to the aphid that are sensitive to rifampicin. One targeted bacterial strain is Buchnera.

Aphids are one of the most important agricultural insect pests. They cause direct feeding damage to the plant and serve as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

Therapeutic Design:

The antibiotic solutions are formulated with 0 (negative control), 1, 10, or 50 μg/ml of rifampicin in 10 mL of sterile water with 0.5% sucrose and essential amino acids.

Experimental Design:

To prepare for the treatment, aphids are grown in a lab environment and medium. In a climate-controlled room (16 h light photoperiod; 60±5% RH; 20±2° C.), fava bean plants are grown in a mixture of vermiculite and perlite at 24° C. with 16 h of light and 8 h of darkness. To limit maternal effects or health differences between plants, 5-10 adults from different plants are distributed among 10 two-week-old plants, and allowed to multiply to high density for 5-7 days. For experiments, second and third instar aphids are collected from healthy plants and divided into treatments so that each treatment receives approximately the same number of individuals from each of the collection plants.

Rifampicin solutions are made by dissolving rifampicin (SIGMA-ALDRICH, 557303) in sterile water with 0.5% sucrose and essential aminoacids. Wells of a 96-well plate are filled with 200 μl of artificial aphid diet (Febvay et al., Canadian Journal of Zoology 66(11):2449-2453, 1988) and the plate is covered with parafilm to make a feeding sachet. Artificial diet is either mixed with sterile water and with 0.5% sucrose and essential aminoacids as a negative control or a rifampicin solution with one of the concentrations of rifampicin. Rifampicin solutions are mixed with artificial diet to get final concentrations of the antibiotic between 1 and 50 μg/mL.

For each replicate treatment, 30-50 second and third instar aphids are placed individually in wells of a 96-well plate and a feeding sachet plate is inverted above them, allowing the insects to feed through the parafilm and keeping them restricted to individual wells. Experimental aphids are kept under the same environmental conditions as aphid colonies. After the aphids are fed for 24 hr, the feeding sachet is replaced with a new one containing sterile artificial diet and a new sterile sachet is provided every 24 h for four days. At the time that the sachet is replaced, aphids are also checked for mortality. An aphid is counted as dead if it had turned brown or is at the bottom of the well and does not move during the observation. If an aphid is on the parafilm of the feeding sachet but not moving, it is assumed to be feeding and alive.

The status of Buchnera in aphid samples is assessed by PCR. Total DNA is isolated from control (non-rifampicin treated) and rifampicin treated individuals using an Insect DNA Kit (OMEGA, Bio-tek) according to the manufacturer's protocol. The primers for Buchnera, forward primer 5′-GTCGGCTCATCACATCC-3′ (SEQ ID NO: 221) and reverse primer 5′-TTCCGTCTGTATTATCTCCT-3′ (SEQ ID NO: 222), are designed based on 23S-5S rRNA sequences obtained from the Buchnera genome (Accession Number: GCA_000009605.1) (Shigenobu et al., Nature 407:81-86, 2000) using Primer 5.0 software (Primer-E Ltd., Plymouth, UK). The PCR amplification cycles included an initial denaturation step at 95° C. for 5 min, 35 cycles of 95° C. for 30 s, 55° C. for 30 s, and 72° C. for 60 s, and a final extension step of 10 min at 72° C. Amplification products from rifampicin treated and control samples are analyzed on 1% agarose gels, stained with SYBR safe, and visualized using an imaging System. Rifampicin treated aphids show a reduction of Buchnera specific genes.

The survival rates of aphids treated with rifampicin solution are compared to the aphids treated with the negative control. The survival rate of aphids treated with rifampicin solution is decreased compared to the control.

### Example 7: Treatment of Varroa Mites that Infect Bees with Rifampicin Solutions

This Example demonstrates the ability to kill or decrease the fitness of Varroa mites by treating them with an antibiotic solution. This Example demonstrates that the effect of oxytetracycline on Varroa mites is mediated through the modulation of bacterial populations endogenous, such as Bacillus, to the Varroa mites that are sensitive to oxytetracycline.

Varroa mites are thought to be a leading cause for the wide spread Colony Collapse Disorder (CCD) that decimates domesticated honey bee colonies of Apis mellifera, around the world. They attach to the bees' abdomen and suck on their blood, depriving them of nutrients, and eventually killing them. While Varroa mites can be killed with chemically synthesized miticides, these types of chemicals must be kept away from comestible honey.

Therapeutic Design:

Oxytetracycline solution is formulated with 0 (negative control), 1, 10, or 50 μg/ml in 10 mL of sterile water with 0.5% sucrose and essential amino acids.

Experimental Design:

To determine whether adult Varroa mites at the reproductive stage have different susceptibility compared to phoretic mites or their offspring because their cuticle is not hardened, Varroa mites living on adult bees, Apis mellifera, and mites associated with larvae and pupae are collected. This assay tests antibiotic solutions on different types of mites and determines how their fitness is altered by targeting endogenous microbes, such as Bacillus.

The brood mites are collected from combs (or pieces of combs) of Varroa mite-infested bee colonies. The mites are collected from cells where bee larvae develop.

Varroa mites are grouped per age of their brood host and assayed separately. The age of the brood is determined based on the morphology and pigmentation of the larva or the pupa as follows: Varroa mites collected from spinning larvae that are small enough to move around their cell are grouped into Group 1; Varroa mites collected from stretched larvae, which are too big to lay in the cell and start to stretch upright with their mouth in the direction of the cell opening, are grouped into Group 2; and Varroa mites collected from pupae are grouped into Group 3. Mites are stored on their host larva or pupa in glass Petri dishes until 50 units are collected. This ensures their feeding routine and physiological status remains unchanged. To prevent mites from straying from their host larva or pupa or climbing onto one another, only hosts at the same development stage are kept in the same dish.

The equipment—a stainless steel ring (56 mm inner diameter, 2-3 mm height) and 2 glass circles (62 mm diameter)—is cleaned with acetone and hexane or pentane to form the testing arena. The oxytetracycline solutions and control solution are applied on the equipment by spraying the glass disks and ring of the arena homogeneously. For this, a reservoir is loaded with 1 ml of the solutions; the distance of the sprayed surface from the bottom end of the tube is set at 11 mm and a 0.0275 inch nozzle is used. The pressure is adjusted (usually in the range 350-500 hPa) until the amount of solution deposited is 1±0.05 mg/cm². The antibiotic solutions are poured in their respective dishes, covering the whole bottom of the dishes, and residual liquid is evaporated under a fume hood. The ring is placed between the glass circles to build a cage. The cages are used within 60 hr of preparation, for not more than three assays, in order to control the exposure of mites to antibiotic solutions. 10 to 15 Varroa mites are introduced in this cage and the equipment pieces are bound together with droplets of melted wax. Mites collected from spinning larvae, stretched larvae, white eyed pupae and dark eyed with white and pale body are used.

After 4 hours, mites are transferred into a clean glass Petri dish (60 mm diameter) with two or three white eye pupae (4-5 days after capping) to feed on. The mites are observed under a dissecting microscope at 4 hr, 24 hr and 48 hr after being treated with the antibiotic or the control solutions, and classified according to the following categories:
Mobile: they walk around when on their legs, whether after being poked by a needle.
Paralyzed: they move one or more appendages, unstimulated or after stimulation, but they cannot move around.
Dead: immobile and do not react to 3 subsequent stimulations.

A sterile toothpick or needle is used to stimulate the mites by touching their legs. New tooth picks or sterile needles are used for stimulating each group to avoid contamination between mite groups.

The assays are carried out at 32.5° C. and 60-70% relative humidity. If the mortality in the controls exceeds 30%, the replicate is excluded. Each experiment is replicated with four series of cages.

The status of Bacillus in Varroa mite groups is assessed by PCR. Total DNA is isolated from control (non-oxytetracycline treated) and oxytetracyline treated individuals (whole body) using a DNA Kit (OMEGA, Bio-tek) according to the manufacturer's protocol. The primers for Bacillus, forward primer 5′-GAGGTAGACGAAGCGACCTG-3′ (SEQ ID NO: 226) and reverse primer 5′-TTCCCTCACGGTACTGGTTC-3′ (SEQ ID NO: 227), are designed based on 23S-5S rRNA sequences obtained from the Bacillus genome (Accession Number: AP007209.1) (Takeno et al., J. Bacteriol. 194(17):4767-4768, 2012) using Primer 5.0 software (Primer-E Ltd., Plymouth, UK). The PCR amplification cycles included an initial denaturation step at 95° C. for 5 min, 35 cycles of 95° C. for 1 min, 59° C. for 1 min, and 72° C. for 2 min, and a final extension step of 5 min at 72° C. Amplification products from oxytetracyline treated and control samples are analyzed on 1% agarose gels, stained with SYBR safe, and visualized using an imaging System.

The survival rates of Varroa mites treated with an oxytetracyline solution are compared to the Varroa mites treated with the negative control.

The survival rate and the mobility of Varroa mites treated with oxytetracyline solution are expected to be decreased compared to the control.

### Example 8: High Throughput Screening (HTS) for Buchnera Targeting Molecules

This Example demonstrates the identification of molecules that target Buchnera.

Experimental Design:

A HTS to identify inhibitors of targeted bacterial strains, Buchnera, uses sucrose fermentation in pH-MMSuc medium (Ymele-Leki et al., PLoS ONE 7(2):e31307, 2012) to decrease the pH of the medium. pH indicators in the medium monitor medium acidification spectrophotometrically through a change in absorbance at 615 nm (A615). A target bacterial strain, Buchnera, derived from a glycerol stock, is plated on an LB-agar plate and incubated overnight at 37° C. A loopful of cells is harvested, washed three times with PBS, and then resuspended in PBS at an optical density of 0.015.

For the HTS, 10 μL of this bacterial cell suspension is aliquoted into the wells of a 384-well plate containing 30 μL of pH-MMSuc medium and 100 nL of a test compound fraction from a natural product library of pre-fractionated extracts (39,314 extracts arrayed in 384-well plates) from microbial sources, such as fungal endophytes, bacterial endophytes, soil bacteria, and marine bacteria, described in (Ymele-Leki et al., PLoS ONE 7(2):e31307, 2012). For each assay, the A615 is measured after incubation at room temperature at 6 hr and 20 hr. This step is automated and validated in the 384-well plate format using an EnVision™ multi-well spectrophotometer to test all fractions from the library. Fractions that demonstrate delayed medium acidification by sucrose fermentation and inhibited cell growth are selected for further purification and identification.

### Example 9: Isolation and Identification of Buchnera Specific Molecules

This Example demonstrates the isolation and identification of an isolate from the fraction described in Example 8 that blocks sucrose fermentation and inhibits cell growth of Buchnera.

Experimental Design:

The fraction described in Example 8 is resuspended in 90% water/methanol and passed over a C18 SPE column to get fraction I. The column is then washed with methanol to get fraction II. Fraction II is separated on an Agilent 1100 series HPLC with a preparative Phenyl-hexyl column (Phenomenex, Luna, 25 cm 610 mm, 5 mm particle size) using an elution buffer with 20% acetonitrile/water with 0.1% formic acid at a flow rate of 2 mL/min for 50 minutes. This yields multiple compounds at different elution times. Spectra for each compound is obtained on an Alpha FT-IR mass spectrometer (Bruker), an Ultrospec™ 5300 pro UV/Visible Spectrophotometer (Amersham Biosciences), and an INOVA 600 MHz nuclear magnetic resonance spectrometer (Varian).

### Example 10: Treatment of Aphids with a Solution of a Buchnera Specific Molecule

This Example demonstrates the ability to kill or decrease the fitness of aphids by treating them with one of the compounds identified in Example 9 through the modulation of bacterial populations endogenous to the aphid that are sensitive to this compound. One targeted bacterial strain is Buchnera.

Therapeutic design:

Each compound from Example 9 is formulated at 0 (negative control), 0.6, 1, 20 or 80 μg/ml in 10 mL of sterile water with 0.5% sucrose and essential amino acids.

Experimental Design:

To prepare for the treatment, aphids are grown in a lab environment and medium. In a climate-controlled room (16 h light photoperiod; 60±5% RH; 20±2° C.), fava bean plants are grown in a mixture of vermiculite and perlite at 24° C. with 16 h of light and 8 h of darkness. To limit maternal effects or health differences between plants, 5-10 adults from different plants are distributed among 10 two-week-old plants, and allowed to multiply to high density for 5-7 days. For experiments, second and third instar aphids are collected from healthy plants and divided into treatments so that each treatment receives approximately the same number of individuals from each of the collection plants.

Wells of a 96-well plate are filled with 200 μl of artificial aphid diet (Febvay et al., Canadian Journal of Zoology 66(11):2449-2453, 1988) and the plate is covered with parafilm to make a feeding sachet. Artificial diet is either mixed with sterile water with 0.5% sucrose and essential amino acids as a negative control or solutions with varying concentrations of the compound.

For each replicate treatment, 30-50 second and third instar aphids are placed individually in wells of a 96-well plate and a feeding sachet plate is inverted above them, allowing the insects to feed through the parafilm and keeping them restricted to individual wells. Experimental aphids are kept under the same environmental conditions as aphid colonies. After the aphids are fed for 24 hr, the feeding sachet is replaced with a new one containing sterile artificial diet and a new sterile sachet is provided every 24 h for 4 days. At the time that the sachet is replaced, aphids are also checked for mortality. An aphid is counted as dead if it had turned brown or is at the bottom of the well and does not move during the observation. If an aphid is on the parafilm of the feeding sachet but not moving, it is assumed to be feeding and alive.

The status of Buchnera in aphid samples is assessed by PCR. Aphids from the negative control and compound 1 treated are first surface-sterilized with 70% ethanol for 1 min, 10% bleach for 1 min and three washes of ultrapure water for 1 min. Total DNA is extracted from each individual (whole body) using an Insect DNA Kit (OMEGA, Bio-tek) according to the manufacturer's protocol. The primers for Buchnera, forward primer 5′-GTCGGCTCATCACATCC-3′ (SEQ ID NO: 221) and reverse primer 5′-TTCCGTCTGTATTATCTCCT-3′ (SEQ ID NO: 222), are designed based on 23S-5S rRNA sequences obtained from the Buchnera genome (Accession Number: GCA_000009605.1) (Shigenobu et al., Nature 407:81-86, 2000) using Primer 5.0 software (Primer-E Ltd., Plymouth, UK). The PCR amplification cycles included an initial denaturation step at 95° C. for 5 min, 35 cycles of 95° C. for 30 s, 55° C. for 30 s, and 72° C. for 60 s, and a final extension step of 10 min at 72° C. Amplification products from compound 1 treated and control samples are analyzed on 1% agarose gels, stained with SYBR safe, and visualized using an imaging System. Reduction of Buchnera specific genes indicates antimicrobial activity of compound 1.

The survival rate of aphids treated with the compound is compared to the aphids treated with the negative control. A decrease in the survival rate of aphids treated with the compound is expected to indicate antimicrobial activity of the compound.

### Example 11: Aphids Treated with an Antibiotic Solution

This Example demonstrates the treatment of aphids with rifampicin, a narrow spectrum antibiotic that inhibits DNA-dependent RNA synthesis by inhibiting a bacterial RNA polymerase. This Example demonstrates that the effect of rifampicin on aphids is mediated through the modulation of bacterial populations endogenous to the aphid that are sensitive to rifampicin. One targeted bacterial strain is Buchnera.

Aphids are agricultural insect pests causing feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design

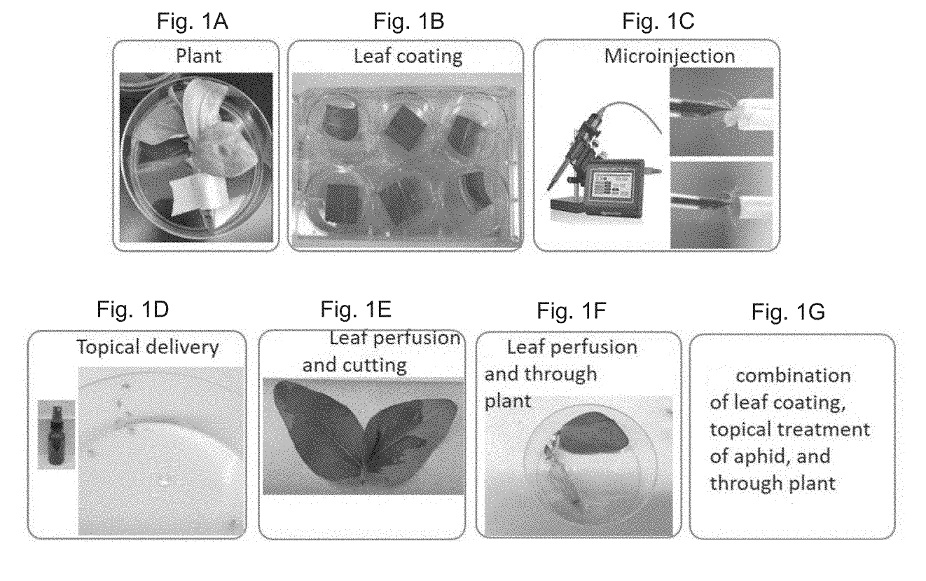

The antibiotic solution was formulated according to the means of delivery, as follows (FIG. 1A-1G):

1) Through the plants: with 0 (negative control) or 100 μg/ml of rifampicin formulated in an artificial diet (based on Akey and Beck, 1971; see Experimental Design) with and without essential amino acids (2 mg/ml histidine, 2 mg/ml isoleucine, 2 mg/ml leucine, 2 mg/ml lysine, 1 mg/ml methionine, 1.62 mg/ml phenylalanine, 2 mg/ml threonine, 1 mg/ml tryptophan, and 2 mg/ml valine).

2) Leaf coating: 100 μl of 0.025% nonionic organosilicone surfactant solvent Silwet L-77 in water (negative control), or 100 μl of 50 μg/ml of rifampicin formulated in solvent solution was applied directly to the leaf surface and allowed to dry.

3) Microinjection: injection solutions were either 0.025% nonionic organosilicone surfactant solvent Silwet L-77 in water (negative control), or 50 μg/ml of rifampicin formulated in solvent solution.

4) Topical delivery: 100 μl of 0.025% nonionic organosilicone surfactant solvent Silwet L-77 (negative control), or 50 μg/ml of rifampicin formulated in solvent solution were sprayed using a 30 mL spray bottle.

5) Leaf injection method A—Leaf perfusion and cutting: leaves were injected with approximately 1 ml of 50 μg/ml of rifampicin in water with food coloring or approximately 1 ml of negative control with water and food coloring. Leaves were cut into 2×2 cm squared pieces and aphids were placed on the leaf pieces.

6) Leaf perfusion and delivery through plant: Leaves were injected with approximately 1 ml of 100 μg/ml of rifampicin in water plus food coloring or approximately 1 ml of negative control with water and food coloring. The stem of injected leaf was then placed into an Eppendorf tube with 1 ml of 100 μg/ml of rifampicin plus water and food coloring or 1 ml of negative control with only water and food coloring.

7) Combination delivery method: a) Topical delivery to aphid and plant: via spraying both aphids and plants with 0.025% nonionic organosilicone surfactant solvent Silwet L-77 in water (negative control) or 100 μg/ml of rifampicin formulated in solvent solution using a 30 mL, b) Delivery through plant: water only (negative control) or 100 μg/ml of rifampicin formulated in water.

### Plant Delivery Experimental Design:

Aphids (LSR-1 strain, Acyrthosiphon pisum) were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first instar aphids were collected from healthy plants and divided into 3 different treatment groups: 1) artificial diet alone without essential amino acids, 2) artificial diet alone without essential amino acids and 100 μg/ml rifampicin, and 3) artificial diet with essential amino acids and 100 μg/ml rifampicin). Each treatment group received approximately the same number of individuals from each of the collection plants.

The artificial diet used was made as previously published (Akey and Beck, 1971 Continuous Rearing of the Pea Aphid, Acyrthosiphon pisum, on a Holidic Diet), with and without the essential amino acids (2 mg/ml histidine, 2 mg/ml isoleucine, 2 mg/ml leucine, 2 mg/ml lysine, 1 mg/ml methionine, 1.62 mg/ml phenylalanine, 2 mg/ml threonine, 1 mg/ml tryptophan, and 2 mg/ml valine), except neither diet included homoserine or beta-alanyltyrosine. The pH of the diets was adjusted to 7.5 with KOH and diets were filter sterilized through a 0.22 μm filter and stored at 4° C. for short term (<7 days) or at −80° C. for long term.

Rifampicin (Tokyo Chemical Industry, LTD) stock solutions were made at 25 mg/ml in methanol, sterilized by passing through a 0.22 μm syringe filter, and stored at −20° C. For treatments (see Therapeutic design), the appropriate amount of stock solution was added to the artificial diet with or without essential amino acids to obtain a final concentration of 100 μg/ml rifampicin. The diet was then placed into a 1.5 ml Eppendorf tube with a fava bean stem with a leaf and the opening of the tube was closed using parafilm. This artificial diet feeding system was then placed into a deep petri dish (Fisher Scientific, Cat# FB0875711) and aphids were applied to the leaves of the plant. For each treatment, 33 aphids were placed onto each leaf. Artificial diet feeding systems were changed every 2-3 days throughout the experiment. Aphids were monitored daily for survival and dead aphids were removed from the deep petri dish housing the artificial feeding system when they were discovered.

In addition, the developmental stage (1^{st}, 2^{nd}, 3^{rd}, 4^{th}, 5^{th} instar) was determined daily throughout the experiment. Once an aphid reached the 4^{th} instar stage, they were given their own artificial feeding system in a deep petri dish so that fecundity could be monitored once they reached adulthood.

For adult aphids, new nymphs were counted daily and then discarded. At the end of the experiments, fecundity was determined as the mean number of offspring produced daily once the aphid reached adulthood. Pictures of aphids were taken throughout the experiment to evaluate size differences between treatment groups.

After 7 days of treatment, DNA was extracted from multiple aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

### Antibiotic Treatment Delays and Stops Progression of Aphid Development

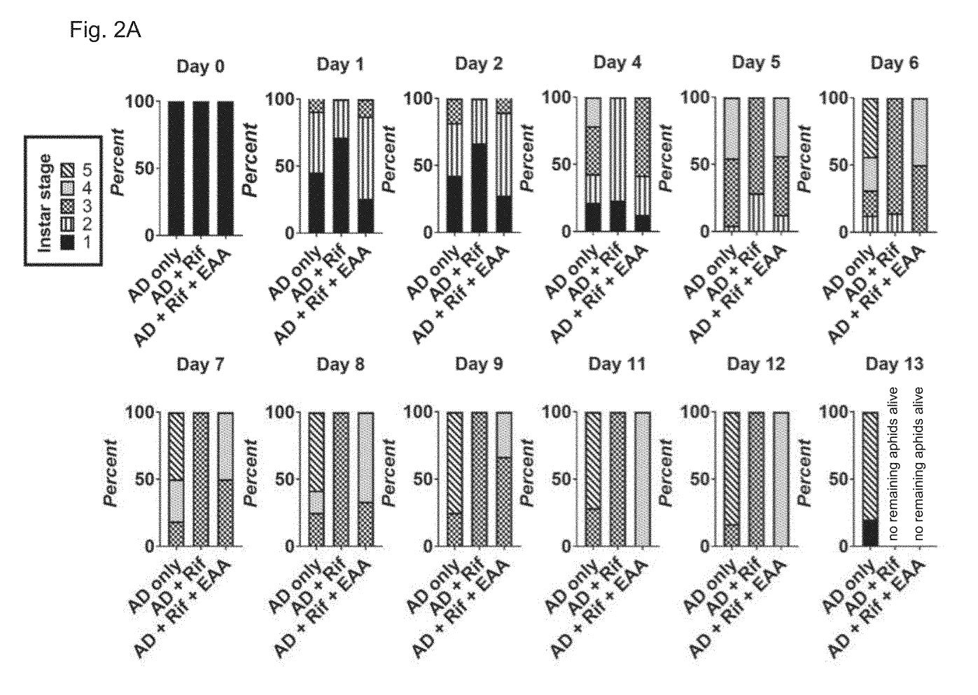

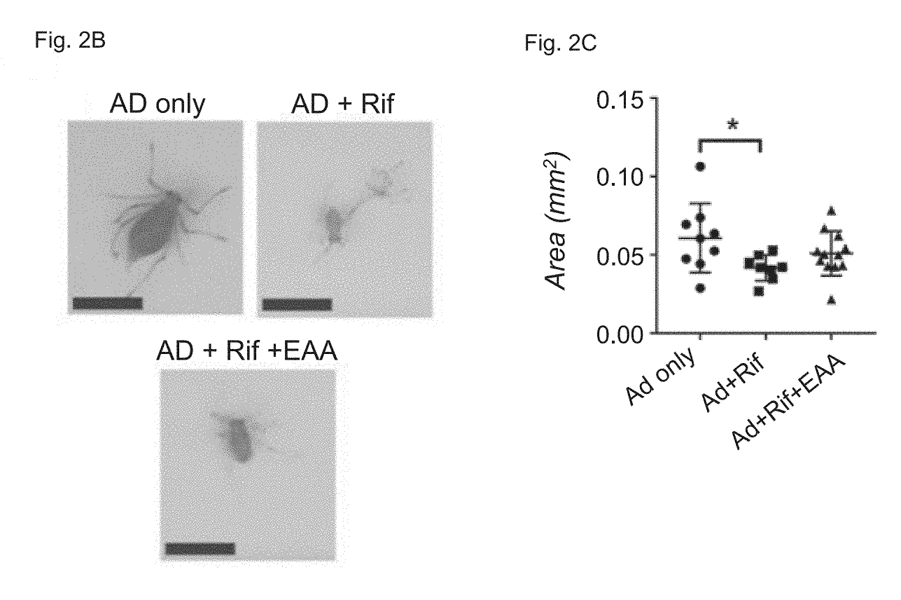

LSR-1 1^{st} instar aphids were divided into three separate treatment groups as defined in Experimental Design (above). Aphids were monitored daily and the number of aphids at each developmental stage was determined. Aphids treated with artificial diet alone without essential amino acids began reaching maturity (5^{th} instar stage) at approximately 6 days (FIG. 2A). Development was delayed in aphids treated with rifampicin, and by 6 days of treatment, most aphids did not mature further than the 3^{rd} instar stage, even after 12 days and their size is drastically affected (FIGS. 2A-2C).

In contrast, aphids treated with artificial diet with rifampicin supplemented with essential amino acids developed faster and to higher instar stages as compared to aphids treated with rifampicin alone, but not as quickly as aphids treated with artificial diet without essential amino acids (FIGS. 2A-2C). These data indicate that treatment with rifampicin impaired aphid development. Adding back essential amino acids partially rescued this defect in development.

### Antibiotic Treatment Increased Aphid Mortality

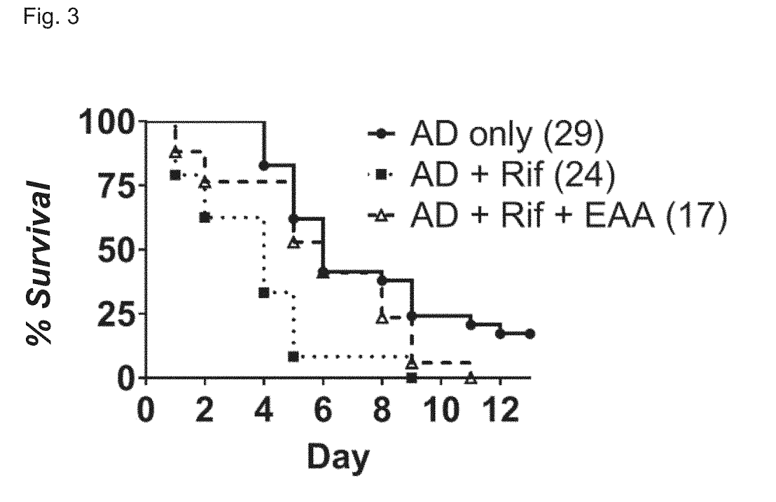

Survival rate of aphids was also measured during the treatments. The majority of the aphids treated with artificial diet alone without essential amino acids were alive at 5 days post-treatment (FIG. 3). After 5 days, aphids began gradually dying, and some survived beyond 13 days post-treatment.

In contrast, aphids treated with rifampicin without essential amino acids had lower survival rates than aphids treated with artificial diet alone (p<0.00001). Rifampicin-treated aphids began dying after 1 day of treatment and all aphids succumbed to treatment by 9 days. Aphids treated with both rifampicin and essential amino acids survived longer than those treated with rifampicin alone (p=0.017). These data indicate that rifampicin treatment affected aphid survival.

### Antibiotic Treatment Decreased Aphid Reproduction

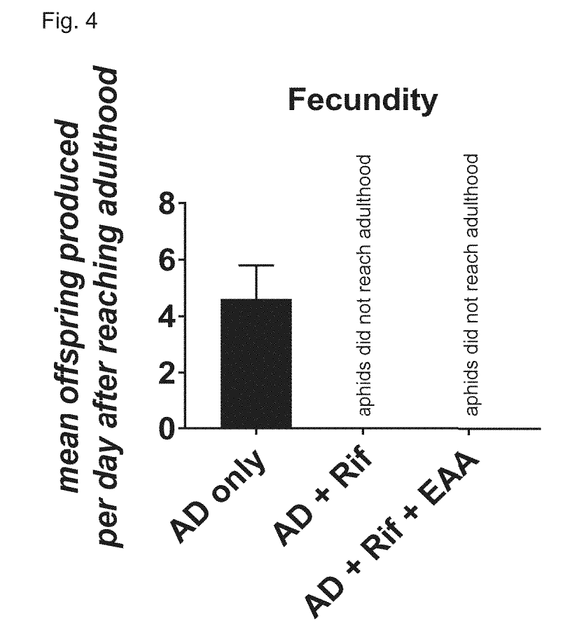

Fecundity was also monitored in aphids during the treatments. By days 7 and 8 post-treatment, the majority of the adult aphids treated with artificial diet without essential amino acids began reproducing. The mean number of offspring produced daily after maturity by aphids treated with artificial diet without essential amino acids was approximately 4 (FIG. 4). In contrast, aphids treated with rifampicin with or without essential amino acids were unable to reach adulthood and produce offspring. These data indicate that rifampicin treatment resulted in a loss of aphid reproduction.

### Antibiotic Treatment Decreased Buchnera in Aphids

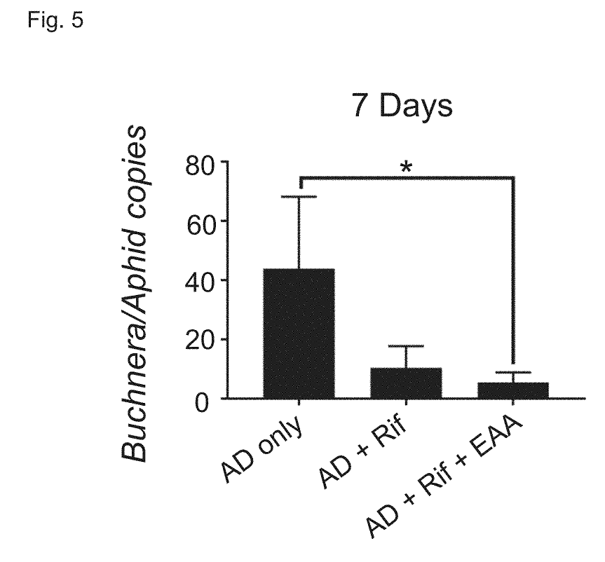

To test whether rifampicin, specifically resulted in loss of Buchnera in aphids, and that this loss impacted aphid fitness, DNA was extracted from aphids in each treatment group after 7 days of treatment and qPCR was performed to determine the Buchnera/aphid copy numbers. Aphids treated with artificial diet alone without essential amino acids had high ratios of Buchnera/aphid DNA copies. In contrast, aphids treated with rifampicin had ˜4-fold less Buchnera/aphid DNA copies (FIG. 5), indicating that rifampicin treatment decreased Buchnera levels.

### Leaf Coating Delivery Experimental Design

Rifampicin stock solution was added to 0.025% of a nonionic organosilicone surfactant solvent, Silwet L-77, to obtain a final concentration of 50 μg/ml rifampicin. Aphids (eNASCO strain, Acyrthosiphon pisum) were grown on fava bean plants as described in a previous Example. For experiments, first instar aphids were collected from healthy plants and divided into 2 different treatment groups: leaves were sprayed with 1) negative control (solvent solution only), 2) 50 μg/ml rifampicin in solvent. Solutions were absorbed onto a 2×2 cm piece of fava bean leaf.

Each treatment group received approximately the same number of individuals from each of the collection plant. For each treatment, 20 aphids were placed onto each leaf. Aphids were monitored daily for survival and dead aphids were removed when they were discovered. In addition, the developmental stage (1^{st}, 2^{nd}, 3^{rd}, 4^{th}, 5^{th} instar, and 5R, representing a reproducing 5^{th} instar) was determined daily throughout the experiment. Pictures of aphids were taken throughout the experiment to evaluate size differences between treatment groups.

After 6 days of treatment, DNA was extracted from multiple aphids from each treatment group and qPCR for quantifying Buchnera levels was done as described in the previous Example.

### Antibiotic Treatment Delivered Through Leaf Coating Delays and Stops Progression of Aphid Development

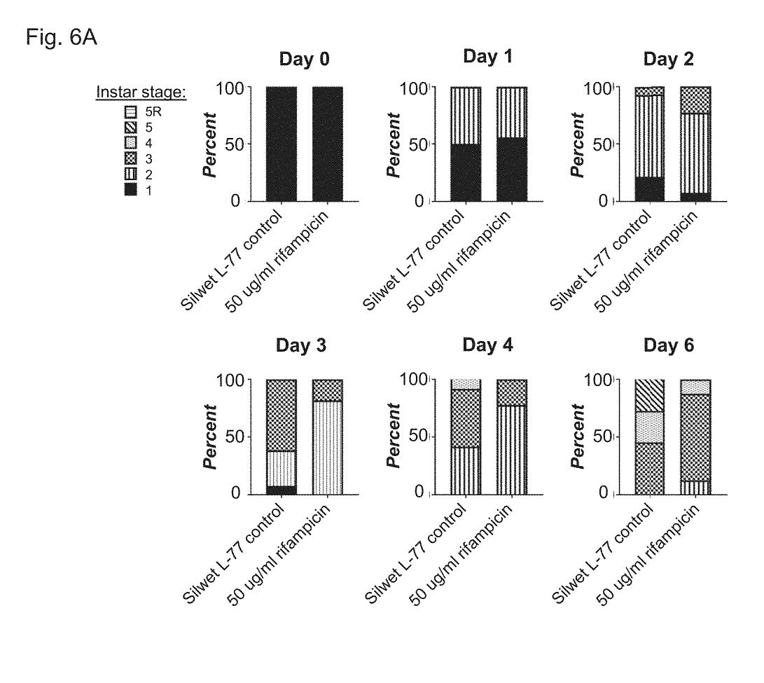

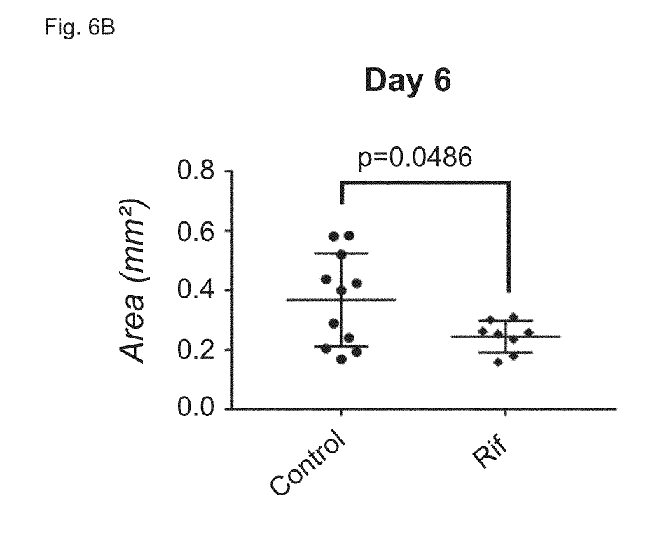

LSR-1 1^{st} instar aphids were divided into two separate treatment groups as defined in the Experimental Design described herein. Aphids were monitored daily and the number of aphids at each developmental stage was determined. Aphids placed on coated leaves treated with control began reaching maturity (5^{th} instar reproducing stage; 5R) at approximately 6 days (FIG. 6A). Development was delayed in aphids placed on coated leaves with rifampicin, and by 6 days of treatment, most aphids did not mature further than the 3^{rd} instar stage, even after 12 days, and their size is drastically affected (FIGS. 6A and 6B).

These data indicate that leaf coating with rifampicin impaired aphid development.

### Antibiotic Treatment Delivered Through Leaf Coating Increased Aphid Mortality

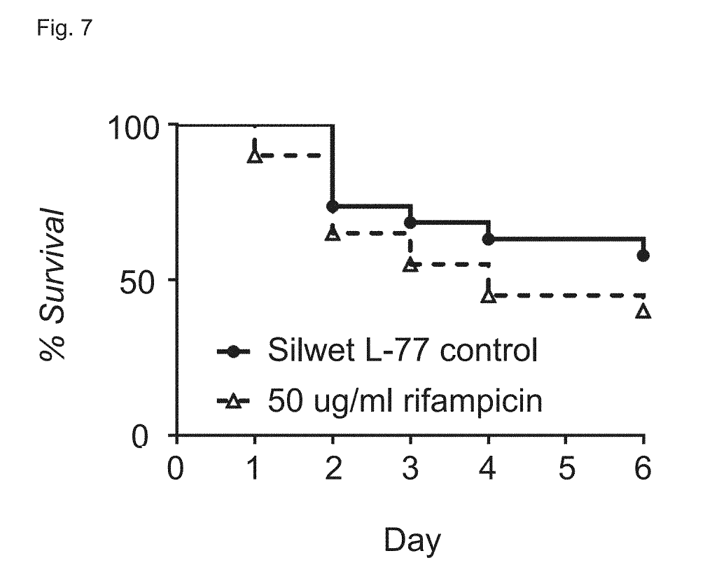

Survival rate of aphids was also measured during the leaf coating treatments. Aphids placed on coated leaves with rifampicin had lower survival rates than aphids placed on coated leaves with the control (FIG. 7). These data indicate that rifampicin treatment delivered through leaf coating affected aphid survival.

### Antibiotic Treatment Delivered Through Leaf Coating Decreased Buchnera in Aphids

To test whether rifampicin delivered through leaf coating, specifically resulted in loss of Buchnera in aphids, and that this loss impacted aphid fitness, DNA was extracted from aphids in each treatment group after 6 days of treatment and qPCR was performed to determine the Buchnera/aphid copy numbers.

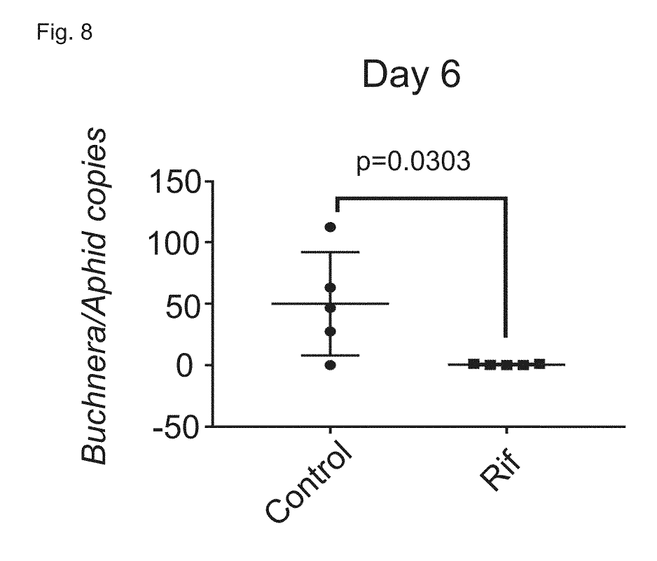

Aphids placed on leaves treated with control had high ratios of Buchnera/aphid DNA copies. In contrast, aphids placed on leaves treated with rifampicin had a drastic reduction of Buchnera/aphid DNA copies (FIG. 8), indicating that rifampicin leaf coating treatment eliminated endosymbiotic Buchnera.

### Microinjection Delivery Experimental Design:

Microinjection was performed using NanoJet III Auto-Nanoliter Injector with the in-house pulled borosilicate needle (Drummond Scientific; Cat#3-000-203-G/XL). Aphids (eNASCO strain, Acyrthosiphon pisum) were grown on fava bean plants as described in a previous Example. Aphids are transferred using a paint brush to a tubing system connected to vacuum (FIG. 1C). The injection site was at the ventral thorax of the aphid. The injection solutions were either the organosilicone surfactant solvent 0.025% Silwet L-77 (Lehle Seeds, Cat No VIS-01) in water (negative control), or 50 μg/ml of rifampicin formulated in solvent solution. The injection volume was 10 nl for nymph and 20 nl for adult (both at a rate of 2 nl/sec). Each treatment group had approximately the same number of individuals injected from each of the collection plants. After injection, aphids were released into a petri dish with fava bean leaves, whose stems are in 2% agar.

Microinjection with Antibiotic Treatment Decreased Buchnera in Aphids

To test whether rifampicin delivered by microinjection results in loss of Buchnera in aphids, and that this loss impacts aphid fitness as demonstrated in previous Examples, DNA was extracted from aphids in each treatment group after 4 days of treatment and qPCR was performed as described in a previous Example to determine the Buchnera/aphid copy numbers.

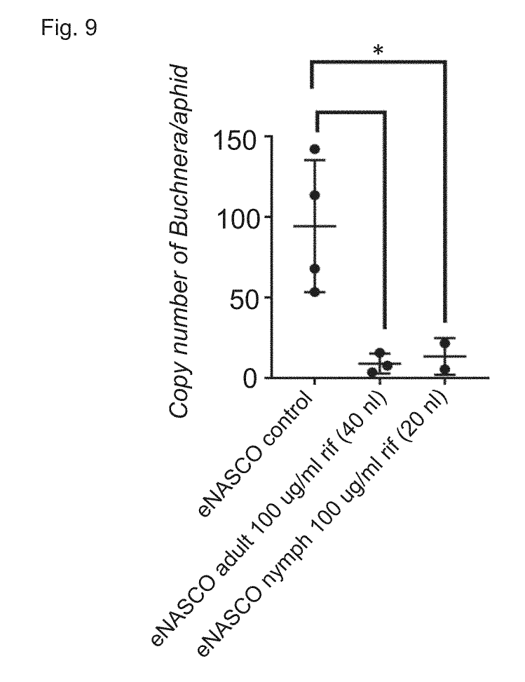

Aphids microinjected with negative control had high ratios of Buchnera/aphid DNA copies. In contrast, aphid nymphs and adults microinjected with rifampicin had a drastic reduction of Buchnera/aphid DNA copies (FIG. 9), indicating that rifampicin microinjection treatment decreased the presence of endosymbiotic Buchnera.

### Topical Delivery Experimental Design:

Aphids (LSR-1 strain, Acyrthosiphon pisum) were grown on fava bean plants as described in a previous Example. Spray bottles were filled with 2 ml of control (0.025% Silwet L-77) or rifampicin solutions (50 μg/ml of in solvent solution). Aphids (number=10) were transferred to the bottom of a clean petri dish and gathered to the corner of the petri dish using a paint brush. Subsequently, aphids were separated into two cohorts and sprayed with ˜100 μl of either control or rifampicin solutions. Immediately after spraying, the petri dish was covered with a lid. Five minutes after spraying, aphids were released into a petri dish with fava bean leaves with stems in 2% agar.

### Topical Delivery of Antibiotic Treatment Decreased Buchnera in Aphids

To test whether rifampicin delivered by topical delivery results in loss of Buchnera in aphids, and that this loss impacts aphid fitness as demonstrated in previous Examples, DNA was extracted from aphids in each treatment group after 3 days of treatment and qPCR as described in a previous Example was performed to determine the Buchnera/aphid copy numbers.

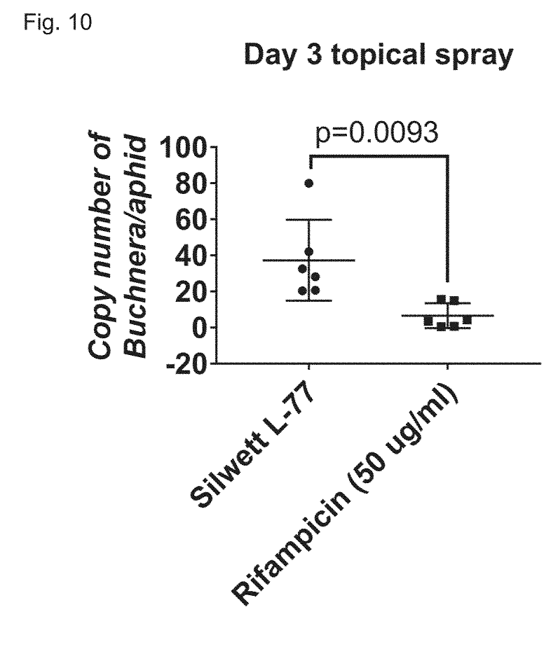

Aphids sprayed with the control solution had high ratios of Buchnera/aphid DNA copies. In contrast, aphids sprayed with rifampicin had a drastic reduction of Buchnera/aphid DNA copies (FIG. 10), indicating that rifampicin treatment delivered through topical treatment decreases the presence of endosymbiotic Buchnera.

### Leaf Injection Method A—Leaf Perfusion and Cutting

Experimental Design:

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 2 different treatment groups: 1) negative control (leaf injected with water plus blue food coloring) and 2) leaf injected with 50 μg/ml rifampicin in water plus blue food coloring. Each treatment group received approximately the same number of individuals from each of the collection plants. For treatment, rifampicin stock solution (25 mg/ml in 100% methanol) was diluted to 50 μg/ml in water plus blue food coloring. The solution was then placed into a 1.5 ml Eppendorf tube with a fava bean stem perfused with the solutions and the opening of the tube was closed using parafilm. This feeding system was then placed into a deep petri dish (Fisher Scientific, Cat# FB0875711) and aphids were applied to the leaves of the plant. For each treatment, 74-81 aphids were placed onto each leaf. The feeding systems were changed every 2-3 days throughout the experiment. Aphids were monitored daily for survival and dead aphids were removed from the deep petri dish when they were discovered. In addition, the developmental stage (1^{st}, 2^{nd}, 3^{rd}, 4^{th}, 5^{th}, and 5R (5^{th} that has reproduced) instar) was determined daily throughout the experiment.

### Antibiotic Treatment Delivered Through Leaf Injection Method A Delays and Stops Progression of Aphid Development

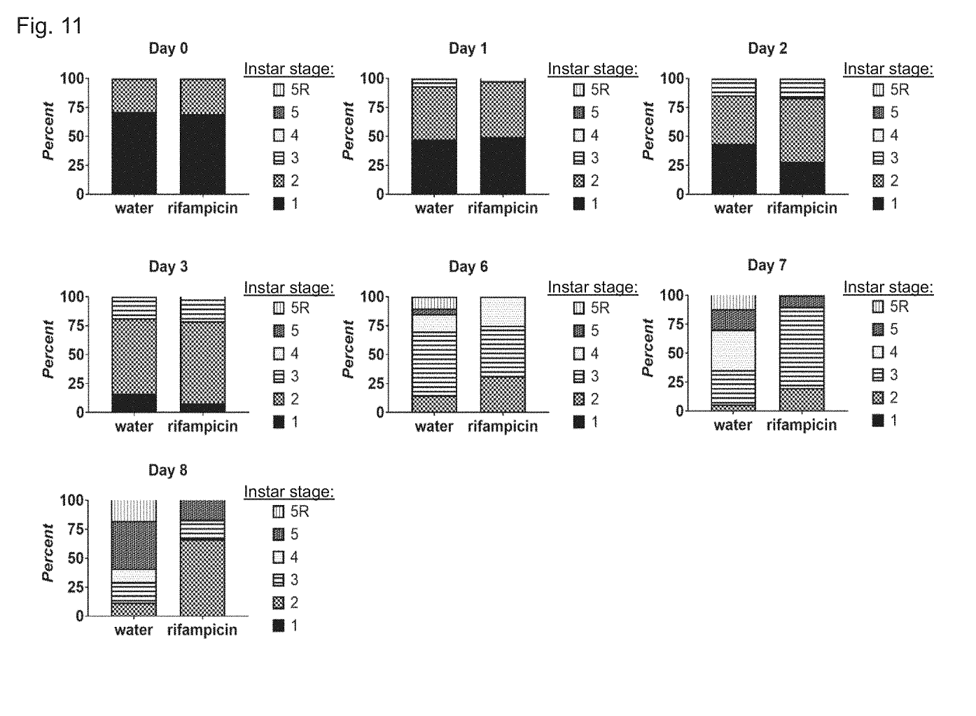

LSR-1 1st and 2nd instar aphids were divided into two separate treatment groups as defined in Leaf injection method A—Leaf perfusion and cutting Experimental Design (described herein). Aphids were monitored daily and the number of aphids at each developmental stage was determined. Aphids treated with water plus food coloring began reaching maturity (5th instar stage) at approximately 6 days (FIG. 11). Development was delayed in aphids feeding on rifampicin injected leaves, and by 6 days of treatment, most aphids did not mature further than the 4th instar stage. Even after 8 days, the development of aphids feeding on rifampicin injected leaves was drastically delayed (FIG. 11). These data indicate that rifampicin treatment via leaf perfusion impaired aphid development.

### Antibiotic Treatment Delivered Through Leaf Injection Method A Increased Aphid Mortality

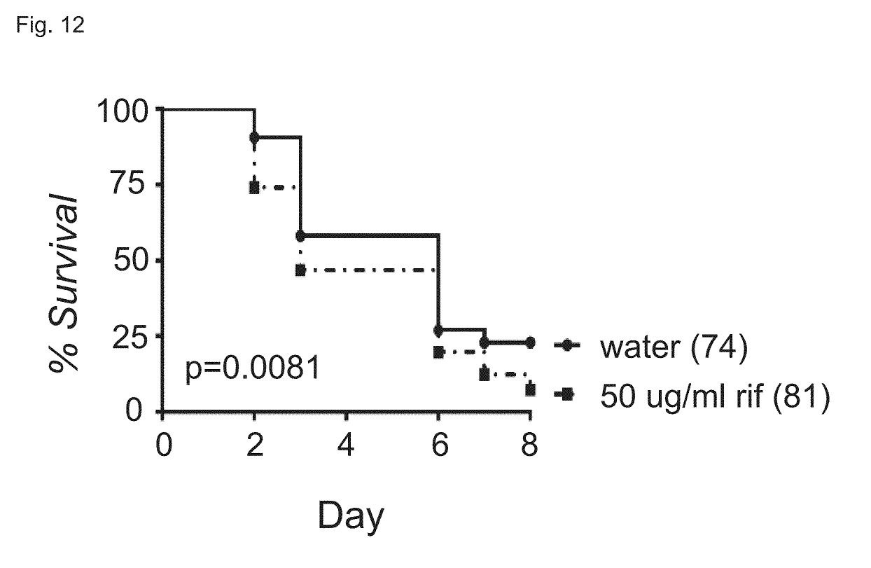

Survival rate of aphids was also measured during the leaf perfusion experiments. Aphids placed on leaves injected with rifampicin had lower survival rates than aphids placed on leaves injected with the control solution (FIG. 12). These data indicate that rifampicin treatment delivered through leaf injection affected aphid survival.

### Antibiotic Treatment Delivered Thorough Leaf Injection Method A Results in Decreased Levels of Buchnera

To test whether rifampicin delivered via leaf perfusion results in loss of Buchnera in aphids, and that this loss impacts aphid fitness as demonstrated in previous Examples, DNA was extracted from aphids in each treatment group after 8 days of treatment and qPCR as described in a previous Example was performed to determine the Buchnera/aphid copy numbers.

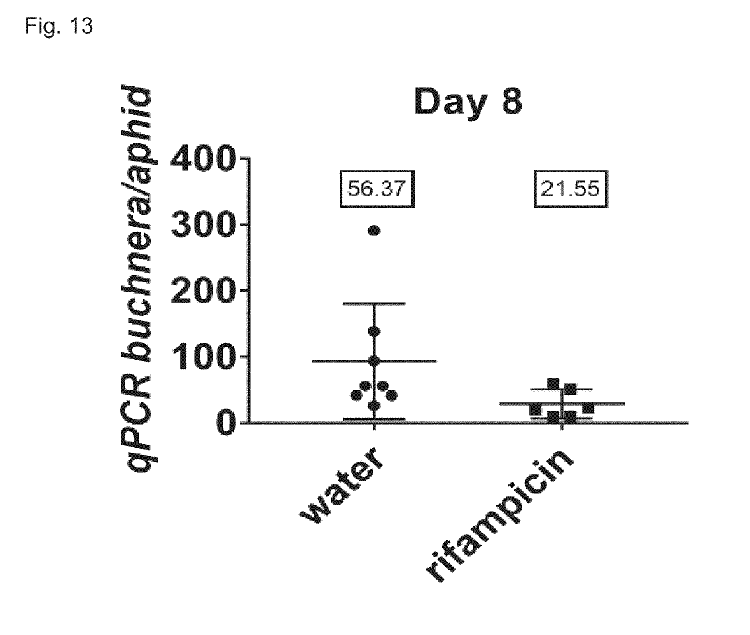

Aphids feeding on leaves injected with the control solution had high ratios of Buchnera/aphid DNA copies. In contrast, aphids feeding on leaves injected with rifampicin had a reduction of Buchnera/aphid DNA copies (FIG. 13), indicating that rifampicin treatment delivered via leaf injection decreases the presence of endosymbiotic Buchnera, as shown in previous Examples, and resulted in a fitness decrease.

### Leaf Perfusion and Delivery Through Plant

Experimental Design:

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 20±2° C.). Prior to being used for aphid rearing, fava bean plants were grown in potting soil at 24° C. with 16 h of light and 8 h of darkness.

To limit maternal effects or health differences between plants, 5-10 adults from different plants were distributed among 10 two-week-old plants, and allowed to multiply to high density for 5-7 days. For experiments, first and second instar aphids were collected from healthy plants and divided into 2 different treatment groups: 1) aphids placed on leaves injected with the negative control solution (water and food coloring) and placed into an Eppendorf tube with the negative control solution, or 2) aphids placed on leaves that were injected with 100 ug/ml rifampicin in water plus food coloring and put into an Eppendorf tube with 100 ug/ml rifampicin in water. Each treatment group received approximately the same number of individuals from each of the collection plants.

For treatment, rifampicin stock solution (25 mg/ml in 100% methanol) was diluted to 100 μg/ml in water plus blue food coloring. The solution was then placed into a 1.5 ml Eppendorf tube with a fava bean stem with a leaf also perfused with the solutions and the opening of the tube was closed using parafilm. This feeding system was then placed into a deep petri dish (Fisher Scientific, Cat# FB0875711) and aphids were applied to the leaves of the plant.

For each treatment, 49-50 aphids were placed onto each leaf. The feeding systems were changed every 2-3 days throughout the experiment. Aphids were monitored daily for survival and dead aphids were removed from the deep petri dish when they were discovered.

In addition, the developmental stage (1^{st}, 2^{nd}, 3^{rd}, 4^{th}, 5^{th}, and 5R (5^{th} that has reproduced) instar) was determined daily throughout the experiment.

### Antibiotic Treatment Delivered Through Leaf Injection and Delivery Through Plant Delays and Stops Progression of Aphid Development

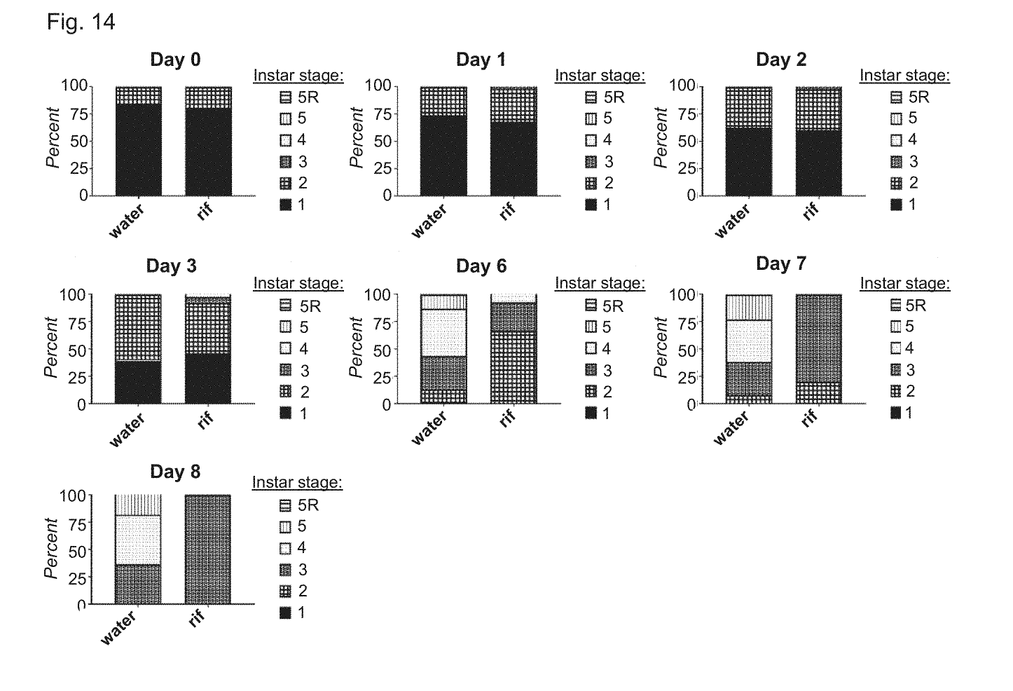

LSR-1 1^{st} and 2^{nd} instar aphids were divided into two separate treatment groups as defined in Leaf perfusion and delivery through plant Experimental Design (described herein). Aphids were monitored daily and the number of aphids at each developmental stage was determined. Aphids treated with the control solution (water plus food coloring only) began reaching maturity (5^{th} instar stage) at approximately 6 days (FIG. 14).

Development was delayed in aphids treated with rifampicin, and by 6 days of treatment, most aphids did not mature further than the 3rd instar stage. Even after 8 days, their development was drastically delayed (FIG. 14). These data indicate that rifampicin treatment via leaf perfusion impaired aphid development.

### Antibiotic Treatment Delivered Through Leaf Injection and Delivery Through Plant Increased Aphid Mortality

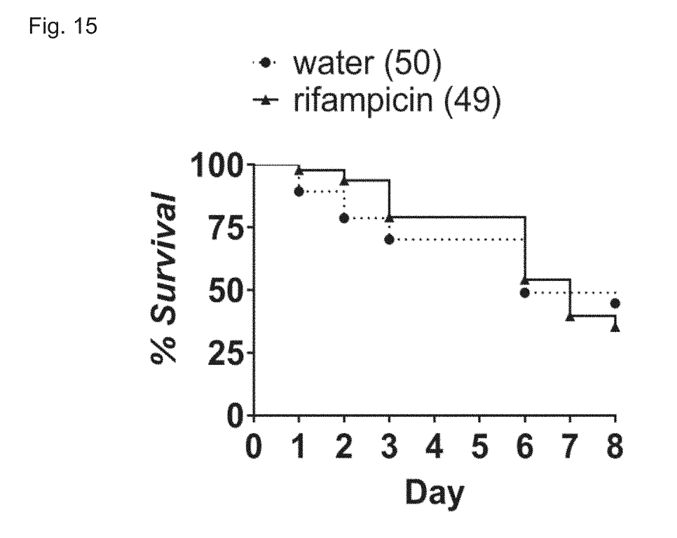

Survival rate of aphids was also measured during the experiments where aphids were treated with either control solution or rifampicin delivered via leaf perfusion and through the plant. Aphids feeding on leaves perfused and treated with rifampicin had lower survival rates than aphids feeding on leaves perfused and treated with the control solution (FIG. 15). These data indicate that rifampicin treatment delivered through leaf perfusion and through the plant negatively impacted aphid survival.

### Antibiotic Treatment Delivered Via Leaf Injection and Through the Plant Results in Decreased Levels of Buchnera

To test whether rifampicin delivered via leaf perfusion and through the plant results in loss of Buchnera in aphids, and that this loss impacts aphid fitness as demonstrated in previous Examples, DNA was extracted from aphids in each treatment group after 6 and 8 days of treatment and qPCR was performed to determine the Buchnera/aphid copy numbers, as described in previous Examples.

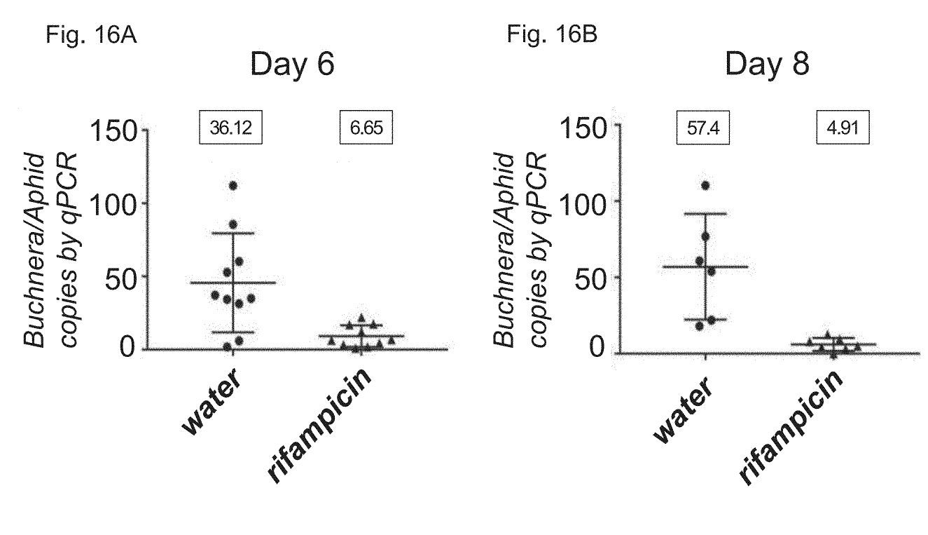

Aphids feeding on leaves injected and treated with the control solution had high ratios of Buchnera/aphid DNA copies. In contrast, aphids feeding on leaves perfused and treated with rifampicin had a statistically significant reduction of Buchnera/aphid DNA copies at both time points (p=0.0037 and p=0.0025 for days 6 and 8, respectively) (FIGS. 16A and 16B), indicating that rifampicin treatment delivered via leaf perfusion and through the plant decreased the presence of endosymbiotic Buchnera, and as shown in previous Examples, and resulted in a fitness decrease.

### Combination Delivery Method

Experimental Design:

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 20±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days.

For experiments, first and second instar aphids were collected from healthy plants and divided into 2 different treatment groups: 1) treatment with Silwet-L77 or water control solutions or 2) treatment with rifampicin diluted in silwet L-77 or water. Each treatment group received approximately the same number of individuals from each of the collection plants. The combination of delivery methods was as follows: a) Topical delivery to aphid and plant by spraying 0.025% nonionic organosilicone surfactant solvent Silwet L-77 (negative control) or 100 μg/ml of rifampicin formulated in solvent solution using a 30 mL spray bottle and b) Delivery through plant with either water (negative control) or 100 μg/ml of rifampicin formulated in water. For treatment, rifampicin stock solution (25 mg/ml in 100% methanol) was diluted to 100 μg/ml in Silwet L-77 (for topical treatment to aphid and coating the leaf) or water (for delivery through plant). The solution was then placed into a 1.5 ml Eppendorf tube with a fava bean stem with a leaf also perfused with the solutions and the opening of the tube was closed using parafilm. This feeding system was then placed into a deep petri dish (Fisher Scientific, Cat# FB0875711) and aphids were applied to the leaves of the plant.

For each treatment, 76-80 aphids were placed onto each leaf. The feeding systems were changed every 2-3 days throughout the experiment. Aphids were monitored daily for survival and dead aphids were removed from the deep petri dish when they were discovered.

In addition, the developmental stage (1^{st}, 2^{nd}, 3^{rd}, 4^{th}, 5^{th}, and 5R (5^{th} that has reproduced) instar) was determined daily throughout the experiment.

### Combination Antibiotic Treatment Delays Aphid Development

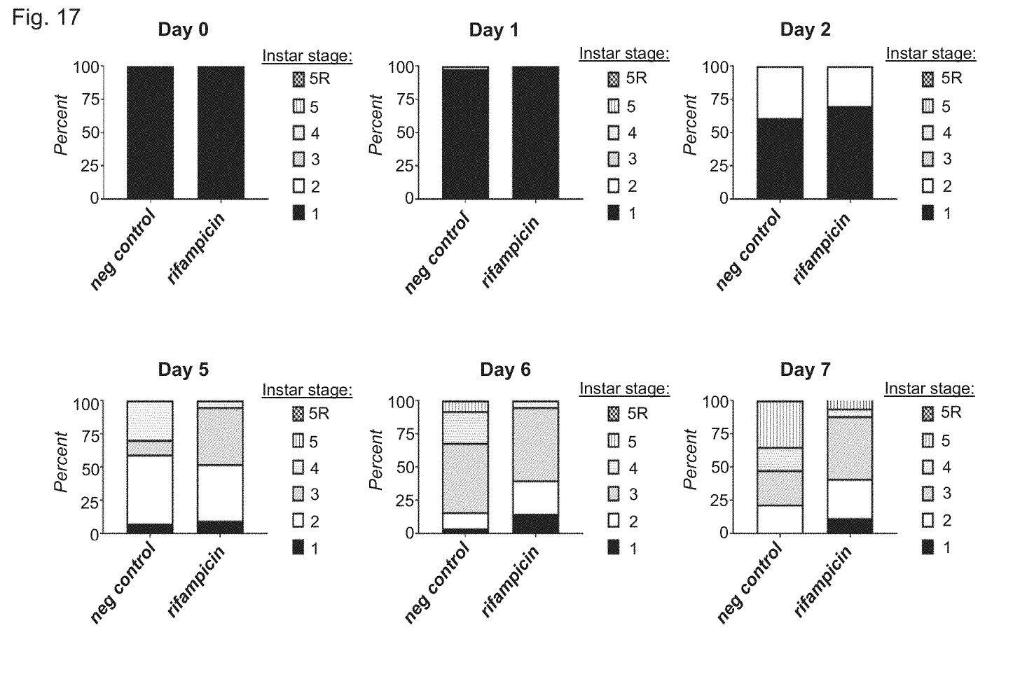

LSR-1 1^{st} and 2^{nd} instar aphids were divided into two separate treatment groups as defined in Combination delivery method Experimental Design (described herein). Aphids were monitored daily and the number of aphids at each developmental stage was determined. Control treated aphids began reaching maturity (5^{th} instar stage) at approximately 6 days (FIG. 17). Development was delayed in aphids treated with rifampicin, and by 6 days of treatment, most aphids did not mature further than the 3^{rd} instar stage, even after 7 days their development was drastically delayed (FIG. 17). These data indicate that a combination of rifampicin treatments impaired aphid development.

### Combination Antibiotic Treatment Results in Increased Aphid Mortality

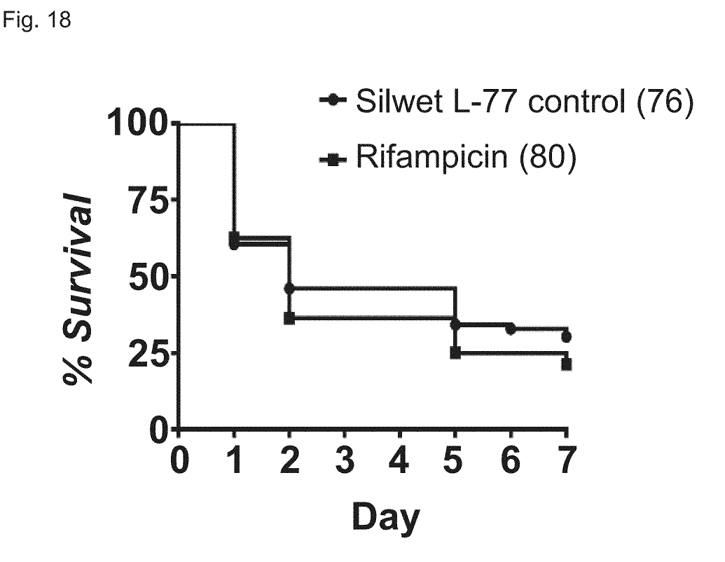

Survival rate of aphids was also measured during the experiments where aphids were treated with a combination of rifampicin treatments. Rifampicin treated aphids had slightly lower survival rates than aphids treated with control solutions (FIG. 18). These data indicate that rifampicin treatment delivered through a combination of treatments affected aphid survival.

### Combination Antibiotic Treatment in Decreased Levels of Buchnera

To test whether rifampicin delivered via a combination of methods results in loss of Buchnera in aphids, and that this loss impacts aphid fitness as demonstrated in previous Examples, DNA was extracted from aphids in each treatment group after 7 days of treatment and qPCR as described in a previous Example was performed to determine the Buchnera/aphid copy numbers.

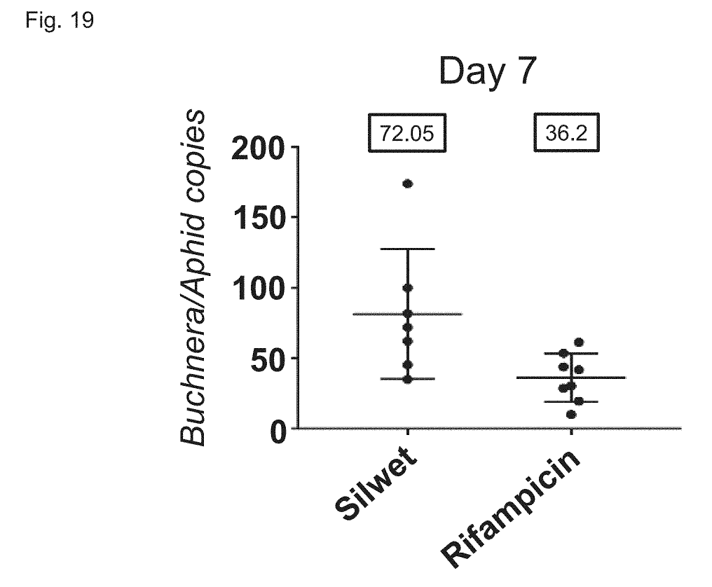

Aphids treated with the control solutions had high ratios of Buchnera/aphid DNA copies. In contrast, aphids treated with rifampicin had a statistically significant and drastic reduction of Buchnera/aphid DNA copies (p=0.227; FIG. 19), indicating that rifampicin treatment delivered via a combination of methods decreases the presence of endosymbiotic Buchnera, and as shown in previous Examples, this resulted in a fitness decrease.

Together this data described in the previous Examples demonstrate the ability to kill and decrease the development, reproductive ability, longevity, and endogenous bacterial populations, e.g., fitness, of aphids by treating them with an antibiotic through multiple delivery methods.

### Example 12: Aphids Treated with a Natural Antimicrobial Polysacharide

This Example demonstrates the treatment of aphids with Chitosan, a natural cationic linear polysaccharide of deacetylated beta-1,4-D-glucosamine derived from chitin. Chitin is the structural element in the exoskeleton of insects, crustaceans (mainly shrimp and crabs) and cell walls of fungi, and the second most abundant natural polysaccharide after cellulose. This Example demonstrates that the effect of chitosan on aphids is mediated through the modulation of bacterial populations endogenous to the aphid that are sensitive to chitosan. One targeted bacterial strain is Buchnera aphidicola.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design

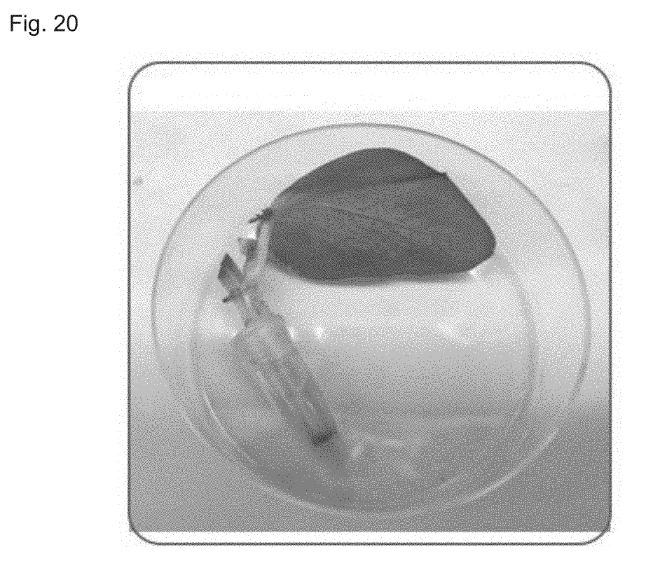

The chitosan solution was formulated using a combination of leaf perfusion and delivery through plants (FIG. 20). The control solution was leaf injected with water+blue food coloring and water in tube. The treatment solution with 300 ug/ml chitosan in water (low molecular weight) via leaf injection (with blue food coloring) and through plant (in Eppendorf tube).

### Leaf Perfusion-Plant Delivery Experimental Design:

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 2 different treatment groups: 1) negative control (water treated), 2) The treatment solution included 300 ug/ml chitosan in water (low molecular weight). Each treatment group received approximately the same number of individuals from each of the collection plants.

Chitosan (Sigma, catalog number 448869-50G) stock solution was made at 1% in acetic acid, sterilized autoclaving, and stored at 4° C. For treatment (see Therapeutic design), the appropriate amount of stock solution was diluted with water to obtain the final treatment concentration of chitosan. The solution was then placed into a 1.5 ml Eppendorf tube with a fava bean stem with a leaf also perfused with the solutions and the opening of the tube was closed using parafilm. This feeding system was then placed into a deep petri dish (Fisher Scientific, Cat# FB0875711) and aphids were applied to the leaves of the plant.

For each treatment, 50-51 aphids were placed onto each leaf. The feeding systems were changed every 2-3 days throughout the experiment. Aphids were monitored daily for survival and dead aphids were removed from the deep petri dish when they were discovered.

In addition, the developmental stage (1^{st}, 2^{nd}, 3^{rd}, 4^{th}, 5^{th}, and 5R (5^{th} that has reproduced) instar) was determined daily throughout the experiment.

After 8 days of treatment, DNA was extracted from multiple aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

There was a Negative Response on Insect Fitness Upon Treatment with the Natural Antimicrobial Polysaccharide

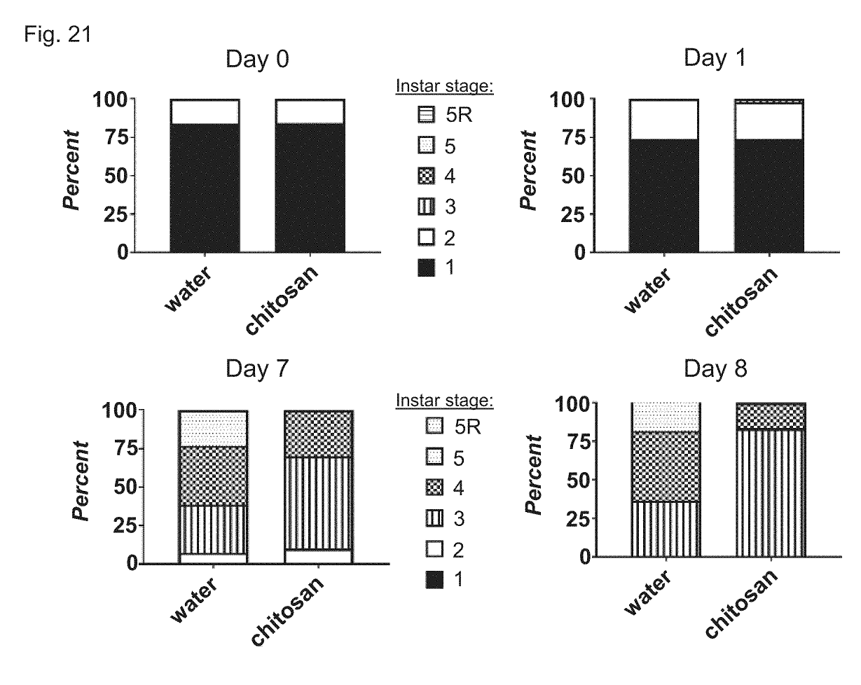

LSR-1 A. pisum 1^{st} and 2^{nd} instar aphids were divided into two separate treatment groups as defined in Experimental Design (above). Aphids were monitored daily and the number of aphids at each developmental stage was determined. Aphids treated with the negative control alone began reaching maturity (5^{th} instar stage) at approximately 6 days (FIG. 21). Development was delayed in aphids treated with chitosan solution, and by 6 days of treatment with chitosan, most aphids did not mature further than the 4^{rd} instar stage. These data indicate that treatment with chitosan delayed and stopped progression of aphid development.

### Chitosan Treatment Increased Aphid Mortality

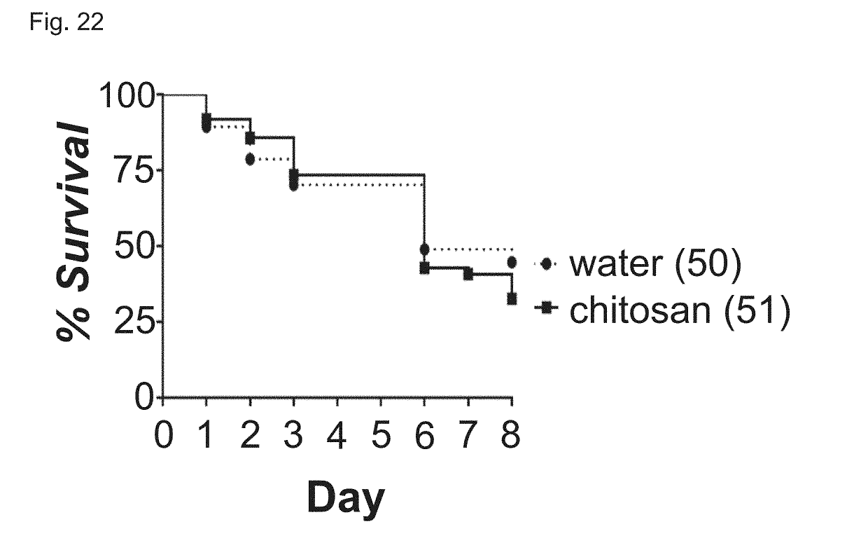

Survival rate of aphids was also measured during the treatments. The majority of the aphids treated with the control alone were alive at 3 days post-treatment (FIG. 22). After 4 days, aphids began gradually dying, and some survived beyond 7 days post-treatment.

In contrast, aphids treated with chitosan solution had lower survival rates than aphids treated with control. These data indicate that there was a decrease in survival upon treatment with the natural antimicrobial polysaccharide.

### Chitosan Treatment Decreased Buchnera in Aphids

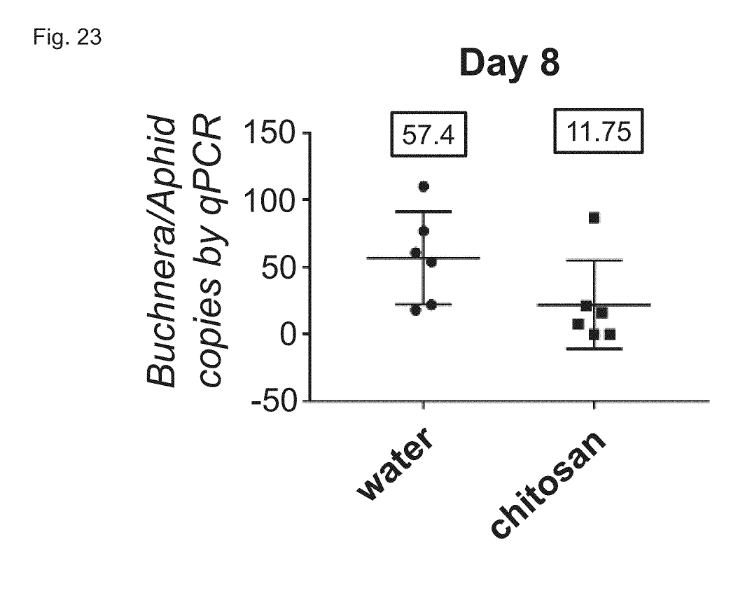

To test whether the chitosan solution treatment, specifically resulted in loss of Buchnera in aphids, and that this loss impacted aphid fitness, DNA was extracted from aphids in each treatment group after 8 days of treatment and qPCR was performed to determine the Buchnera/aphid copy numbers. Aphids treated with control alone had high ratios of Buchnera/aphid DNA copies. In contrast, aphids treated with 300 ug/ml chitosan in water had ˜5-fold less Buchnera/aphid DNA copies (FIG. 23), indicating that chitosan treatment decreased Buchnera levels.

Together this data described previously demonstrated the ability to kill and decrease the development, longevity, and endogenous bacterial populations, e.g., fitness, of aphids by treating them with a natural antimicrobial polysaccharide.

### Example 13: Aphids Treated with Nisin, a Natural Antimicrobial Peptide

This Example demonstrates the treatment of aphids with the natural, “broad spectrum”, polycyclic antibacterial peptide produced by the bacterium Lactococcus lactis that is commonly used as a food preservative. The antibacterial activity of nisin is mediated through its ability to generate pores in the bacterial cell membrane and interrupt bacterial cell-wall biosynthesis through a specific lipid II interaction. This Example demonstrates that the negative effect of nisin on aphid fitness is mediated through the modulation of bacterial populations endogenous to the aphid that are sensitive to nisin. One targeted bacterial strain is Buchnera aphidicola.

Aphids are agricultural insect pests causing direct feeding damage to the plant and serve as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design:

Nisin was formulated using a combination of leaf perfusion and delivery through plants. The control solution (water) or treatment solution (nisin) was injected into the leaf and placed in the Eppendorf tube. The treatment solutions consisted of 1.6 or 7 mg/ml nisin in water.

### Leaf Perfusion-Plant Delivery Experimental Design:

LSR-1 aphids, Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 2 different treatment groups: 1) negative control (water treated), 2) nisin treated with either 1.6 or 7 mg/ml nisin in water. Each treatment group received approximately the same number of individuals from each of the collection plants.

For treatment (see Therapeutic design), nisin (Sigma, product number: N5764) solution was made at 1.6 or 7 mg/ml (w/v) in water and filter sterilized using a 0.22 um syringe filter. The solution was then injected into the leaf of the plant and the stem of the plant was placed into a 1.5 ml Eppendorf tube. The opening of the tube was closed using parafilm. This feeding system was then placed into a deep petri dish (Fisher Scientific, Cat# FB0875711) and aphids were applied to the leaves of the plant.

For each treatment, 56-59 aphids were placed onto each leaf. The feeding systems were changed every 2-3 days throughout the experiment. Aphids were monitored daily for survival and dead aphids were removed from the deep petri dish when they were discovered.

In addition, the developmental stage (1st, 2nd, 3rd, 4th, 5th, and 5R (5th instar aphids that are reproducing) instar) was determined daily throughout the experiment.

After 8 days of treatment, DNA was extracted from the remaining aphids in each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

There was a Dose-Dependent Negative Response on Insect Fitness Upon Treatment with Nisin

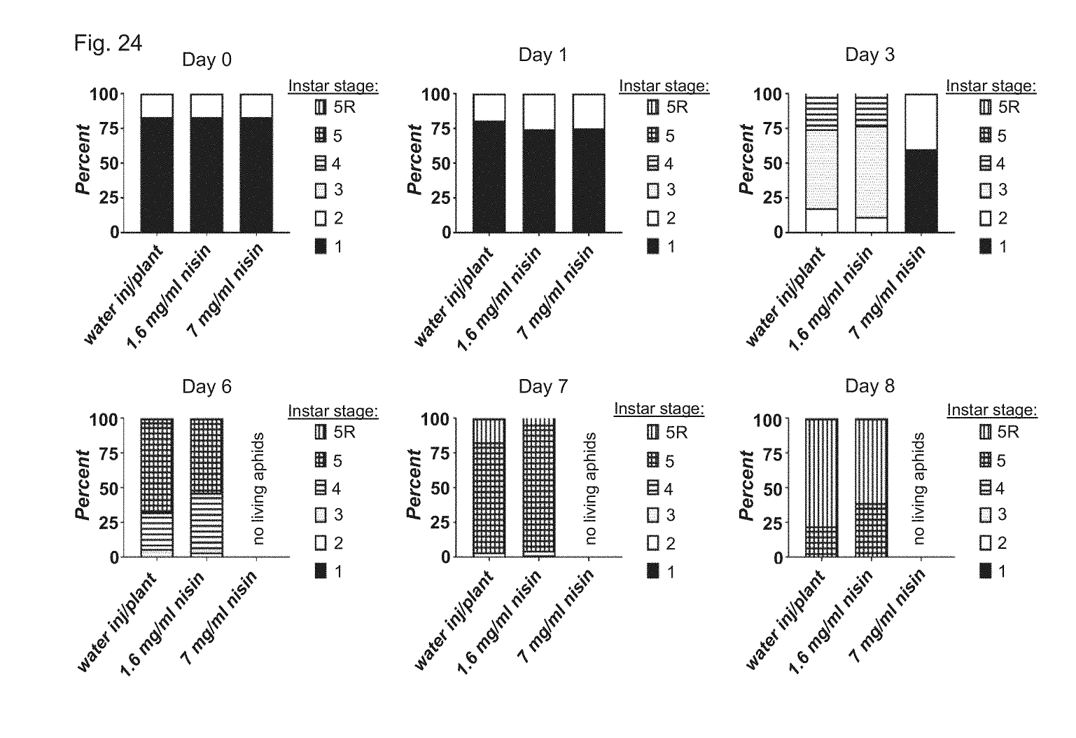

LSR-1 A. pisum 1st and 2nd instar aphids were divided into three separate treatment groups as defined in Experimental Design (above). Aphids were monitored daily and the number of aphids at each developmental stage was determined. Aphids treated with the negative control solution (water) began reaching maturity (5th instar stage) at approximately 6 days, and reproducing (5R stage) by 7 days (FIG. 24). Development was severely delayed in aphids treated with 7 mg/ml nisin. Aphids treated with 7 mg/ml nisin only developed to the 2nd instar stage by day 3, and by day 6, all aphids in the group were dead (FIG. 24). Development was slightly delayed in aphids treated with the lower concentration of nisin (1.6 mg/ml) and at each time point assessed, there were more less developed aphids compared to water-treated controls (FIG. 24). These data indicate that treatment with nisin delayed and stopped progression of aphid development and this delay in development was dependent on the dose of nisin administered.

However, it is important to note that treatment with 7 mg/ml of nisin also had a negative impact on the health of the leaves used in the assay. Shortly after leaf perfusion of 7 mg/ml of nisin it started turning brown. After two days, the leaves perfused with 7 mg/ml turned black. There was no noticeable difference in the condition of the leaves treated with 1.6 mg/ml nisin.

Treatment with Nisin Resulted in Increased Aphid Mortality

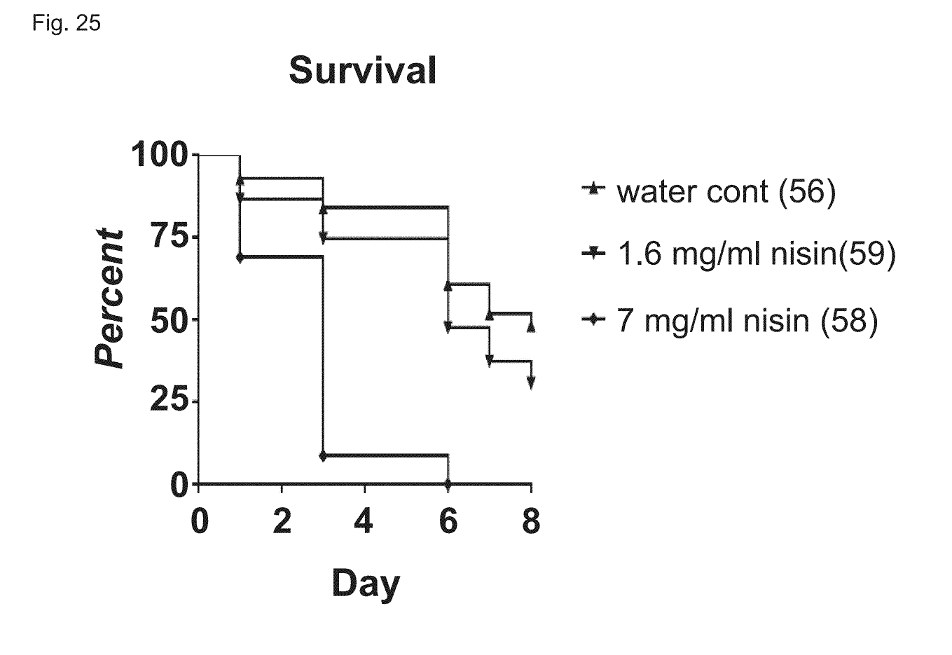

Survival rate of aphids was also measured during the treatments. Approximately 50% of aphids treated with the control alone survived the 8-day experiment (FIG. 25). In contrast, survival was significantly less for aphids treated with 7 mg/ml nisin (p<0.0001, by Log-Rank Mantel Cox test), and all aphids in this group succumbed to the treatment by 6 days (FIG. 25). Aphids treated with the lower dose of nisin (1.6 mg/ml) had higher mortality compared to control treated aphids, although the difference did not reach statistical significance (p=0.0501 by Log-Rank Mantel Cox test). These data indicate that there was a dose-dependent decrease in survival upon treatment with nisin.

Treatment with Nisin Resulted in Decreased Buchnera in Aphids

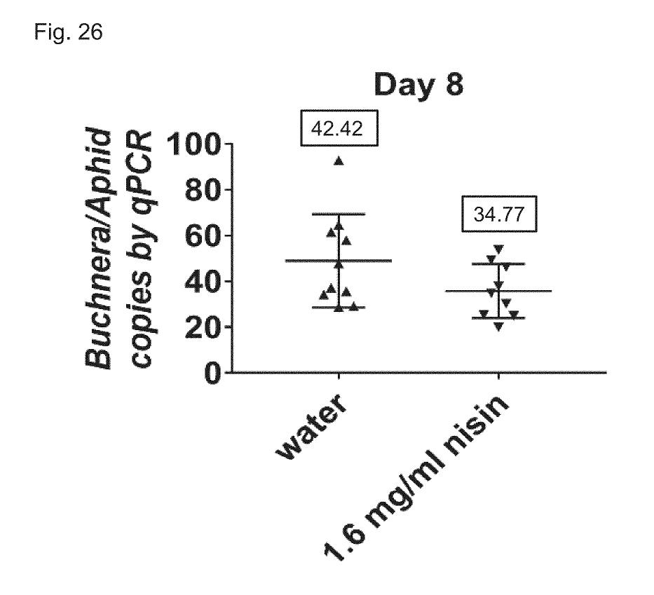

To test whether treatment with nisin, specifically resulted in loss of Buchnera in aphids, and that this loss impacted aphid fitness, DNA was extracted from aphids in each treatment group after 8 days of treatment and qPCR was performed to determine the Buchnera/aphid copy numbers. Aphids treated with control alone had high ratios of Buchnera/aphid DNA copies. In contrast, aphids treated with 1.6 mg/ml nisin had ˜1.4-fold less Buchnera/aphid DNA copies after 8 days of treatment (FIG. 26). No aphids were alive in the group treated with 7 mg/ml nisin, and therefore, Buchnera/aphid DNA copies was not assessed in this group. These data indicate that nisin treatment decreased Buchnera levels.

Together this data described previously demonstrate the ability to kill and decrease the development, longevity, and endogenous bacterial populations, e.g., fitness, of aphids by treating them with the antimicrobial peptide nisin.

### Example 14: Aphids Treated with Levulinic Acid Decreases Insect Fitness

This Example demonstrates the treatment of aphids with levulinic acid, a keto acid produced by heating a carbohydrate with hexose (e.g. wood, starch, wheat, straw, or cane sugar) with the addition of a dilute mineral acid reduces insect fitness. Levulinic acid has previously been tested as an antimicrobial agent against Escherichia coli and Salmonella in meat production (Carpenter et al., 2010, Meat Science; Savannah G. Hawkins, 2014, University of Tennessee Honors Thesis). This Example demonstrates that the effect of levulinic acid on aphids is mediated through the modulation of bacterial populations endogenous to the aphid that are sensitive to levulinic acid. One targeted bacterial strain is Buchnera aphidicola.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design:

The levulinic acid was formulated using a combination of leaf perfusion and delivery through plants. The control solution was leaf injected with water and water was placed in the Eppendorf tube. The treatment solutions included 0.03 or 0.3% levulinic acid in water via leaf injection and through plant (in Eppendorf tube).

### Leaf Perfusion-Plant Delivery Experimental Design:

eNASCO aphids, Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 2 different treatment groups: 1) negative control (water treated), 2) The treatment solution included 0.03 or 0.3% levulinic acid in water. Each treatment group received approximately the same number of individuals from each of the collection plants.

For treatment (see Therapeutic design), levulinic acid (Sigma, product number: W262706) was diluted to either 0.03 or 0.3% in water. The solution was then placed into a 1.5 ml Eppendorf tube with a fava bean stem with a leaf also perfused with the solutions and the opening of the tube was closed using parafilm. This feeding system was then placed into a deep petri dish (Fisher Scientific, Cat# FB0875711) and aphids were applied to the leaves of the plant.

For each treatment, 57-59 aphids were placed onto each leaf. The feeding systems were changed every 2-3 days throughout the experiment. Aphids were monitored daily for survival and dead aphids were removed from the deep petri dish when they were discovered.

In addition, the developmental stage (1^{st}, 2^{nd}, 3^{rd}, 4^{th}, and 5^{th} instar) was determined daily throughout the experiment.

After 7 of treatment, DNA was extracted from the remaining aphids in each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch groES_18F (CATGATCGTGTGCTTGTTAAG; SEQ ID NO: 228) and Buch groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

There was a Dose-Dependent Negative Response on Insect Fitness Upon Treatment with Levulinic Acid

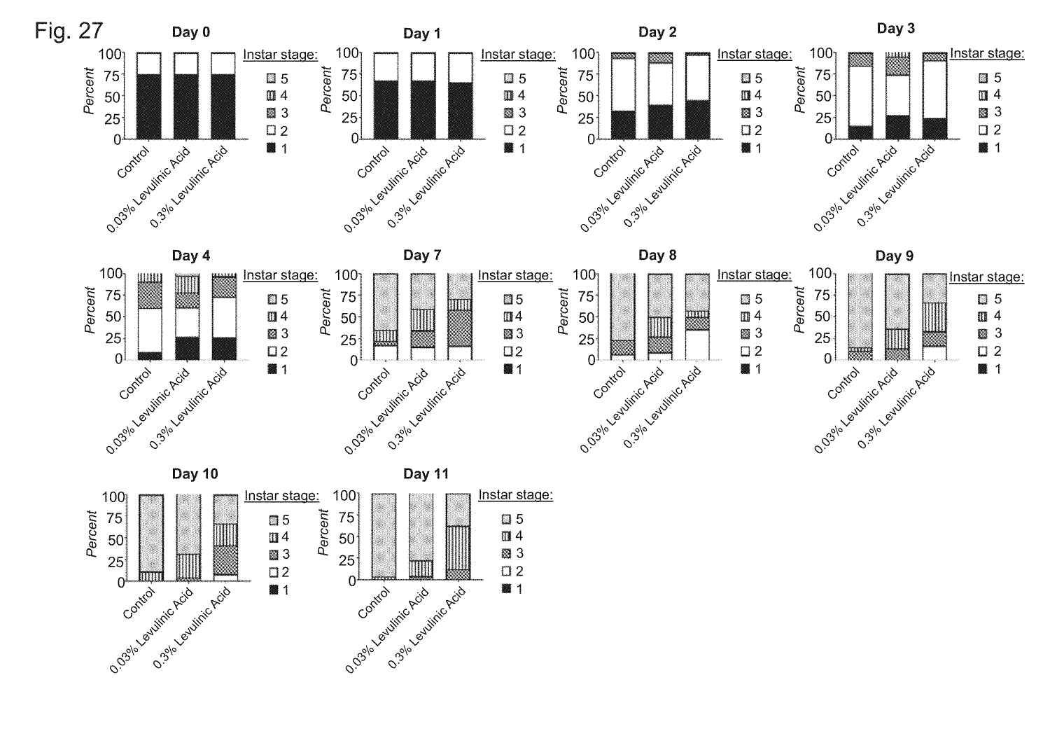

eNASCO A. pisum 1^{st} and 2^{nd} instar aphids were divided into three separate treatment groups as defined in Experimental Design (above). Aphids were monitored daily and the number of aphids at each developmental stage was determined. Aphids treated with the negative control alone began reaching maturity (5^{th} instar stage) at approximately 7 days (FIG. 27). Development was delayed in aphids treated with levulinic acid and by 11 days post-treatment, nearly all control treated aphids reached maturity while ˜23 and 63% aphids treated with 0.03 and 0.3% levulinic acid, respectively, did not mature further than the 4^{rd} instar stage. These data indicate that treatment with levulinic acid delayed and stopped progression of aphid development and this delay in development is dependent on the dose of levulinic acid administered.

Treatment with Levulinic Acid Results in Increased Aphid Mortality

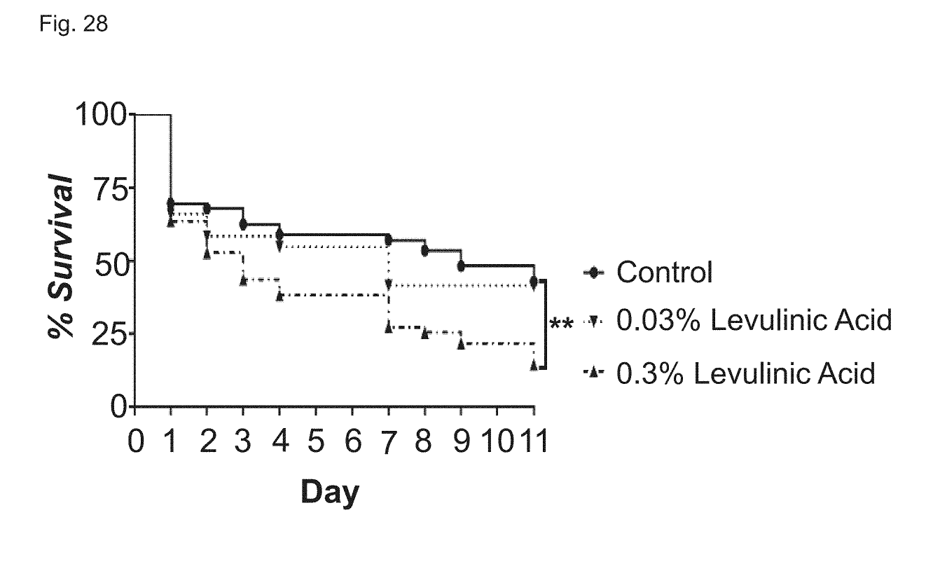

Survival rate of aphids was also measured during the treatments. Approximately 50% of aphids treated with the control alone survived the 11-day experiment (FIG. 28). In contrast, survival was significantly less for aphids treated with 0.3% levulinic acid (p<0.01). Aphids treated with the low dose of levulinic acid (0.03%) had higher mortality compared to control treated aphids, although the difference did not reach statistical significance. These data indicate that there was a dose-dependent decrease in survival upon treatment with levulinic acid.

Treatment with Levulinic Acid Results in Decreased Buchnera in Aphids

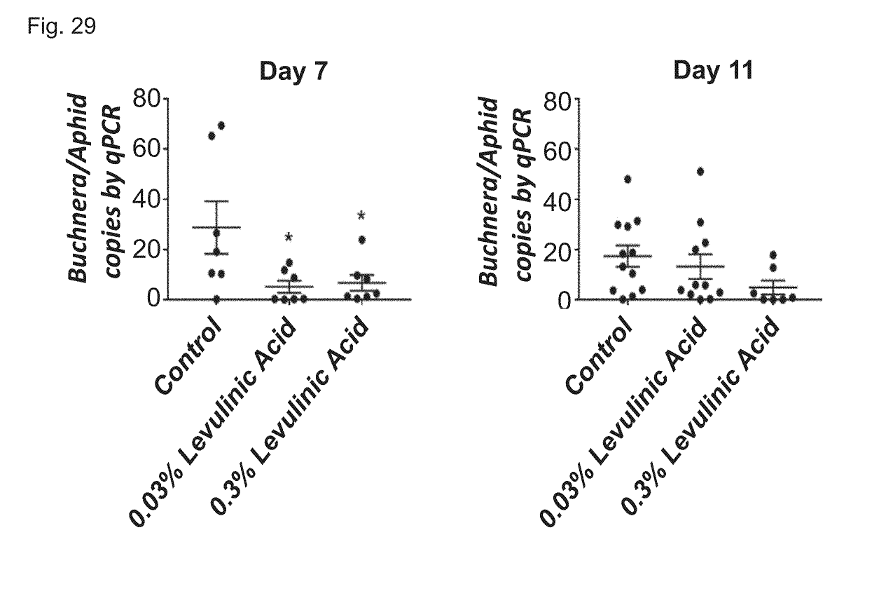

To test whether treatment with levulinic acid, specifically resulted in loss of Buchnera in aphids, and that this loss impacted aphid fitness, DNA was extracted from aphids in each treatment group after 7 days of treatment and qPCR was performed to determine the Buchnera/aphid copy numbers. Aphids treated with control alone had high ratios of Buchnera/aphid DNA copies. In contrast, aphids treated with 0.03 or 0.3% levulinic acid in water had ˜6-fold less Buchnera/aphid DNA copies after 7 days of treatment (FIG. 29, left panel). These data indicate that levulinic acid treatment decreased Buchnera levels.

Together this data described previously demonstrated the ability to kill and decrease the development, longevity, and endogenous bacterial populations, e.g., fitness, of aphids by treating them with levulinic acid.

### Example 15: Aphids Treated with a Plant Derived Secondary Metabolite Solution

This Example demonstrates the treatment of aphids with gossypol acetic acid, a natural phenol derived from the cotton plant (genus Gossypium) that permeates cells and acts as an inhibitor for several dehydrogenase enzymes. This Example demonstrates that the effect of gossypol on aphids is mediated through the modulation of bacterial populations endogenous to the aphid that are sensitive to gossypol. One targeted bacterial strain is Buchnera aphidicola.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

Therapeutic Design:

The gossypol solution was formulated depending on the delivery method:

1) Through the plants: with 0 (negative control) or 0.5%, 0.25%, and 0.05% of gossypol formulated in an artificial diet (based on Akey and Beck, 1971; see Experimental Design) without essential amino acids (histidine, isoleucine, leucine, lysine, methionine, phenylalanine, threonine, tryptophan, and valine).

2) Microinjection: injection solutions were either 0.5% of gossypol or artificial diet only (negative control).

Plant Delivery Experimental Design:

Aphids (either eNASCO (which harbor both Buchnera and Serratia primary and secondary symbionts, respectively) or LSR-1 (which harbor only Buchnera) strains, Acyrthosiphon pisum) were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 4 different treatment groups: 1) artificial diet alone without essential amino acids, 2) artificial diet alone without essential amino acids and 0.05% of gossypol, 3) artificial diet alone without essential amino acids and 0.25% of gossypol, and 4) artificial diet alone without essential amino acids and 0.5% of gossypol. Each treatment group received approximately the same number of individuals from each of the collection plants.

The artificial diet used was made as previously published (Akey and Beck, 1971 Continuous Rearing of the Pea Aphid, Acyrthosiphon pisum, on a Holidic Diet), with and without the essential amino acids (2 mg/ml histidine, 2 mg/ml isoleucine, 2 mg/ml leucine, 2 mg/ml lysine, 1 mg/ml methionine, 1.62 mg/ml phenylalanine, 2 mg/ml threonine, 1 mg/ml tryptophan, and 2 mg/ml valine), except neither diet included homoserine or beta-alanyltyrosine. The pH of the diets was adjusted to 7.5 with KOH and diets were filter sterilized through a 0.22 μm filter and stored at 4° C. for short term (<7 days) or at −80° C. for long term.

Gossypol acetic acid (Sigma, Cat#G4382-250MG) stock solution was made at 5% in methanol and sterilized by passing through a 0.22 μm syringe filter, and stored at 4° C. For treatments (see Therapeutic design), the appropriate amount of stock solution was added to the artificial diet to obtain the different final concentrations of gossypol. The diet was then placed into a 1.5 ml Eppendorf tube with a fava bean stem with a leaf and the opening of the tube was closed using parafilm. This feeding system was then placed into a deep petri dish (Fisher Scientific, Cat# FB0875711) and aphids were applied to the leaves of the plant.

For each treatment, 15-87 aphids were placed onto each leaf. Artificial diet feeding systems were changed every 2-3 days throughout the experiment. Aphids were monitored daily for survival and dead aphids were removed from the deep petri dish housing the artificial feeding system when they were discovered.

In addition, the developmental stage (1^{st}, 2^{nd}, 3^{rd}, 4^{th}, 5^{th}, and 5R (5^{th} that has reproduced) instar) was determined daily throughout the experiment. Once an aphid reached the 4^{th} instar stage, they were given their own artificial feeding system in a deep petri dish so that fecundity could be monitored once they reached adulthood.

For adult aphids, new nymphs were counted daily and then discarded. At the end of the experiments, fecundity was measured in two ways: 1) the mean day at which the first offspring for the treatment group was determined and 2) the mean number of offspring produced daily once the aphid reached adulthood. Pictures of aphids were taken throughout the experiment to evaluate size differences between treatment groups.

After 5 or 13 days of treatment, DNA was extracted from multiple aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

There was a Dose-Dependent Negative Response on Insect Fitness Upon Treatment with the Allelochemical Gossypol

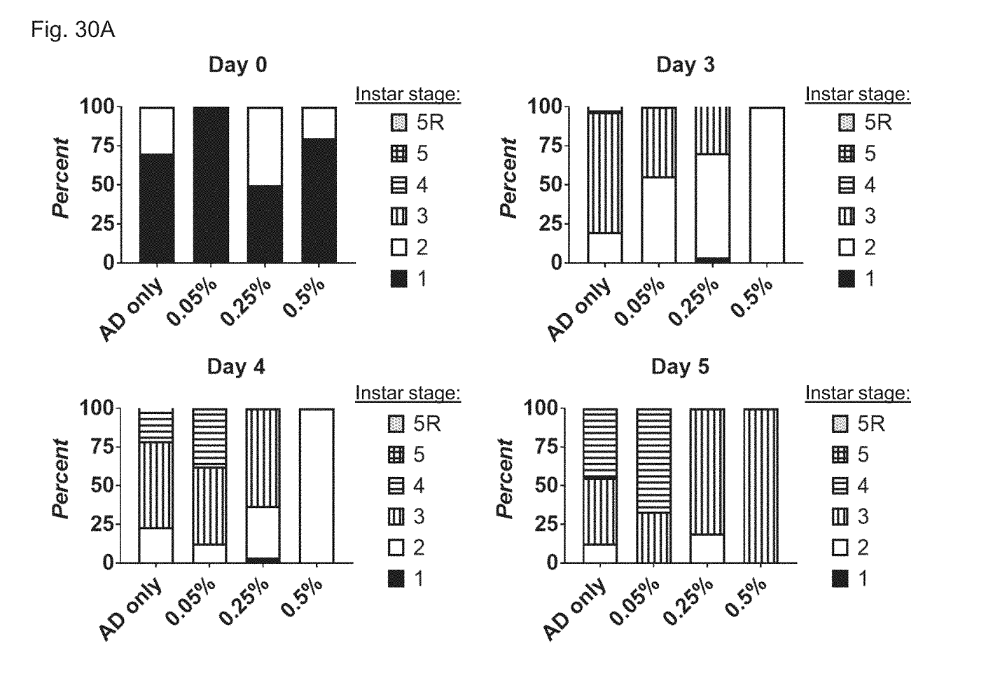

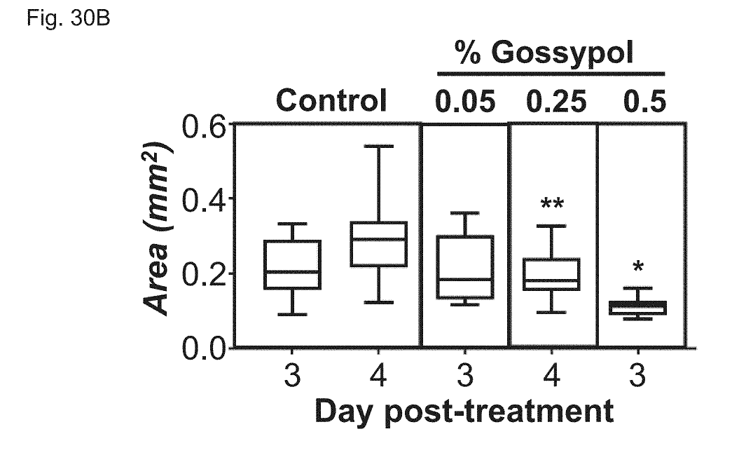

eNASCO and LSR-1 A. pisum 1^{st} and 2^{nd} instar aphids were divided into four separate treatment groups as defined in Experimental Design (described herein). Aphids were monitored daily and the number of aphids at each developmental stage was determined. Aphids treated with artificial diet alone began reaching maturity (5^{th} instar stage) at approximately 3 days (FIG. 30A). Development was delayed in aphids treated with gossypol, and by 5 days of treatment with 0.5% of gossypol, most aphids did not mature further than the 3^{rd} instar stage, and their size is also affected (FIGS. 30A and 30B). These data indicate that treatment with gossypol delayed and stopped progression of aphid development, and that this response was dose dependent.

### Gossypol Treatment Increased Aphid Mortality

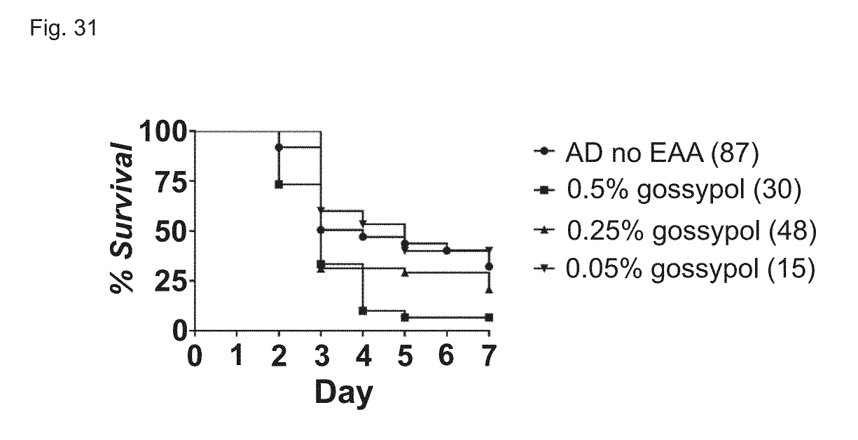

Survival rate of aphids was also measured during the treatments. The majority of the aphids treated with artificial diet alone without essential amino acids were alive at 2 days post-treatment (FIG. 31). After 4 days, aphids began gradually dying, and some survived beyond 7 days post-treatment.

In contrast, aphids treated with 0.25 (not significantly different than control, p=0.2794) and 0.5% of gossypol had lower survival rates than aphids treated with artificial diet alone, with 0.5% gossypol treatment being significantly different than AD no EAA control (p=0.00²). 0.5% gossypol-treated aphids began dying after 2 days of treatment and all aphids succumbed to treatment by 4 days. Aphids treated with 0.25% survived a bit longer than those treated with 0.5% but were also drastically affected. These data indicate that there was a dose-dependent decrease in survival upon treatment with the allelochemical gossypol.

### Gossypol Treatment Decreased Aphid Reproduction

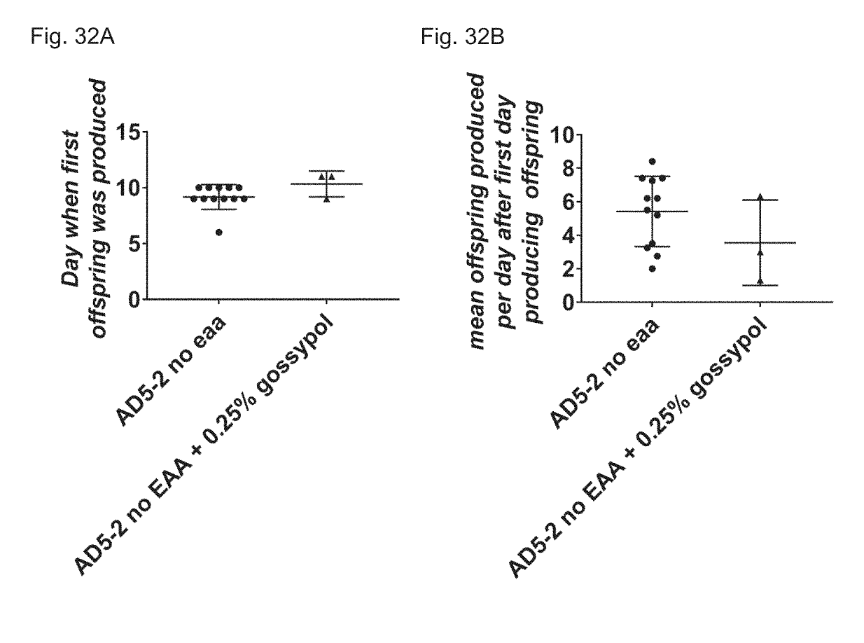

Fecundity was also monitored in aphids during the treatments. By days 7 and 8 post-treatment, the majority of the adult aphids treated with artificial diet without essential amino acids began reproducing. The mean number of offspring produced daily after maturity by aphids treated with artificial diet without essential amino acids was approximately 5 (FIGS. 32A and 32B).

In contrast, aphids treated with 0.25% of gossypol show a reduction to reach adulthood and produce offspring. These data indicate that gossypol treatment resulted in a decrease of aphid reproduction.

### Gossypol Treatment Decreased Buchnera in Aphids

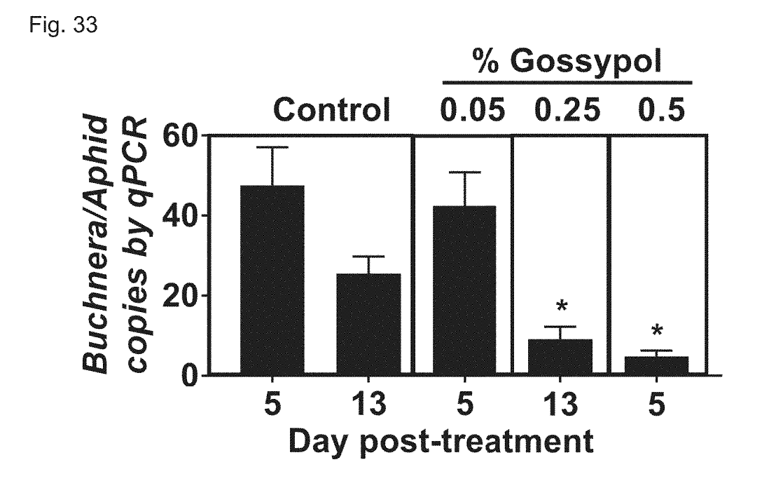

To test whether different concentrations of gossypol, specifically resulted in loss of Buchnera in aphids, and that this loss impacted aphid fitness, DNA was extracted from aphids in each treatment group after 5 or 13 days of treatment and qPCR was performed to determine the Buchnera/aphid copy numbers. Aphids treated with artificial diet alone without essential amino acids (control) had high ratios of Buchnera/aphid DNA copies. In contrast, aphids treated with 0.25 and 0.5% of gossypol had ˜4-fold less Buchnera/aphid DNA copies (FIG. 33), indicating that gossypol treatment decreased Buchnera levels, and that this decrease was concentration dependent.

### Microinjection Delivery Experimental Design:

Microinjection was performed using NanoJet III Auto-Nanoliter Injector with the in-house pulled borosilicate needle (Drummond Scientific; Cat#3-000-203-G/XL). Aphids (LSR-1 strain, A. pisum) were grown on fava bean plants as described in a previous Example. Each treatment group had approximately the same number of individuals injected from each of the collection plants. Nymph aphids (<3^{rd} instar stage) were transferred using a paint brush to a tubing system connected to vacuum and microinjected into the ventral thorax with 20 nl of either artificial diet without essential amino acids (negative control) or 0.05% of gossypol solution in artificial diet without essential amino acids. After injection, aphids were placed in a deep petri dish with a fava bean leaf with stem in 2% agar.

Microinjection with Antibiotic Treatment Decreased Buchnera in Aphids

To test whether gossypol delivered by microinjection results in loss of Buchnera in aphids, and that this loss impacts aphid fitness as demonstrated in previous Examples, DNA was extracted from aphids in each treatment group after 4 days of treatment and qPCR was performed as described in a previous Example to determine the Buchnera/aphid copy numbers.

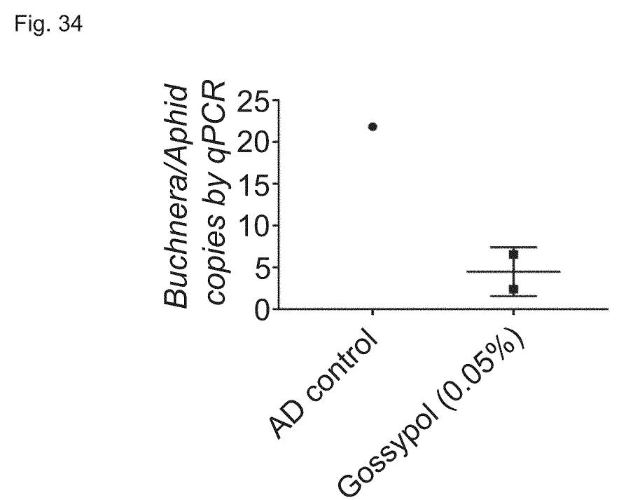

Aphids microinjected with negative control had high ratios of Buchnera/aphid DNA copies. In contrast, aphid nymphs and adults microinjected with gossypol had a drastic reduction of Buchnera/aphid DNA copies (FIG. 34), indicating that gossypol microinjection treatment decreases the presence of endosymbiotic Buchnera, and as shown in previous Examples this resulted in a fitness decrease.

Together this data described in the previous Examples demonstrated the ability to kill and decrease the development, reproductive ability, longevity, and endogenous bacterial populations, e.g., fitness, of aphids by treating them with plant secondary metabolite solution through multiple delivery methods.

### Example 16: Aphids Treated with Natural Plant Derived Antimicrobial Compound, Trans-Cinnemaldehyde

This Example demonstrates the treatment of aphids with trans-cinnemaldehyde, a natural aromatic aldehyde that is the major component of bark extract of cinnamon (Cinnamomum zeylandicum) results in decreased fitness. Trans-cinnemaldehyde has been shown to have antimicrobial activity against both gram-negative and gram-positive organisms, although the exact mechanism of action of its antimicrobial activity remains unclear. Trans-cinnemaldehyde may damage bacterial cell membranes and inhibit of specific cellular processes or enzymes (Gill and Holley, 2004 Applied Environmental Microbiology). This Example demonstrates that the effect of trans-cinnemaldehyde on aphids was mediated through the modulation of bacterial populations endogenous to the aphid that are sensitive to trans-cinnemaldehyde. One targeted bacterial strain is Buchnera aphidicola.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design:

Trans-cinnemaldehyde was diluted to 0.05%, 0.5%, or 5% in water and was delivered through leaf perfusion (˜1 ml was injected into leaves) and through plants.

### Experimental Design:

Aphids (LSR-1 (which harbor only Buchnera) strains, Acyrthosiphon pisum) were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into four different treatment groups: 1) water treated controls, 2) 0.05% trans-cinnemaldehyde in water, 3) 0.5% trans-cinnemaldehyde in water, and 4) 5% trans-cinnemaldehyde in water. Each treatment group received approximately the same number of individuals from each of the collection plants.

Trans-cinnemaldehyde (Sigma, Cat#C80687) was diluted to the appropriate concentration in water (see Therapeutic design), sterilized by passing through a 0.22 μm syringe filter, and stored at 4° C. Fava bean leaves were injected with approximately 1 ml of the treatment and then the leaf was placed in a 1.5 ml Eppendorf tube containing the same treatment solution. The opening of the tube where the fava bean stem was placed was closed using parafilm. This treatment feeding system was then placed into a deep petri dish (Fisher Scientific, Cat# FB0875711) and aphids were applied to the leaves of the plant.

For each treatment, 40-49 aphids were placed onto each leaf. Treatment feeding systems were changed every 2-3 days throughout the experiment. Aphids were monitored daily for survival and dead aphids were removed from the deep petri dish housing the treatment feeding system when they were discovered.

In addition, the developmental stage (1^{st}, 2^{nd}, 3^{rd}, 4^{th}, 5^{th}, and 5R (5^{th} that has reproduced) instar) was determined daily throughout the experiment.

After 3 days of treatment, DNA was extracted from the remaining living aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

There was a Dose-Dependent Negative Response on Insect Fitness Upon Treatment with the Natural Antimicrobial Trans-Cinnemaldehyde

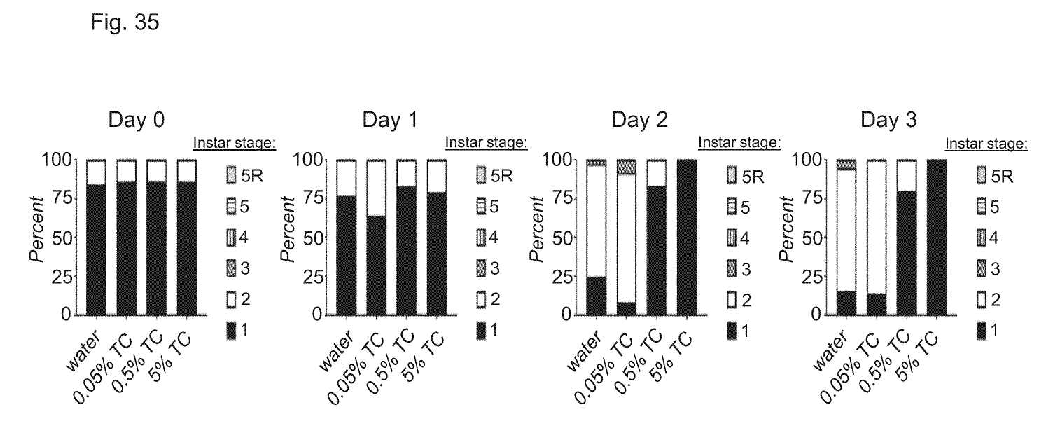

LSR-1 A. pisum 1^{st} and 2^{nd} instar aphids were divided into four separate treatment groups as defined in Experimental Design (described herein). Aphids were monitored daily and the number of aphids at each developmental stage was determined. Aphids treated with water alone began reaching the 3^{rd} instar stage at 3 days post-treatment (FIG. 35). Development was delayed in aphids treated with trans-cinnemaldehyde, and by 3 days of treatment with each the three of the trans-cinnemaldehyde concentrations, none of the aphids matured past the second instar stage (FIG. 35). Moreover, all the aphids treated with the highest concentration of trans-cinnemaldehyde (5%) remained at the 1^{st} instar stage throughout the course of the experiment. These data indicate that treatment with trans-cinnemaldehyde delays and stops progression of aphid development, and that this response is dose dependent.

### Trans-Cinnemaldehyde Treatment Increased Aphid Mortality

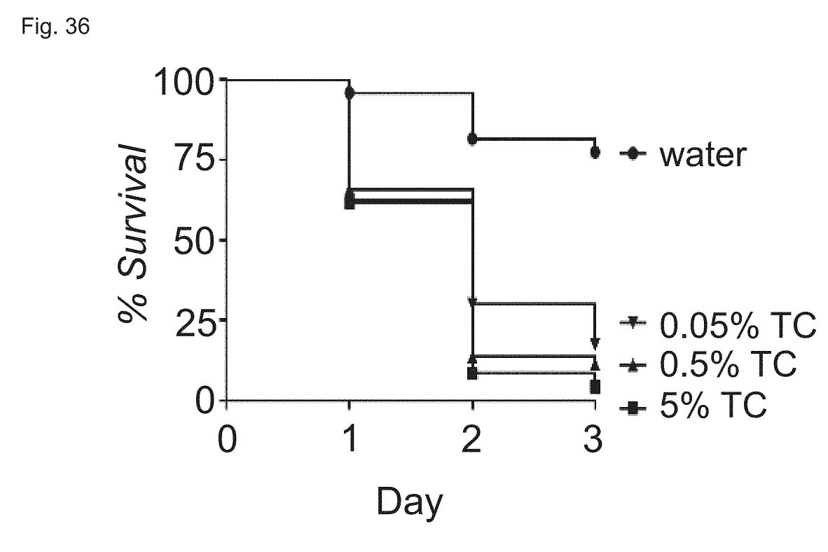

Survival rate of aphids was also measured during the treatments. Approximately 75 percent of the aphids treated with water alone were alive at 3 days post-treatment (FIG. 36). In contrast, aphids treated with 0.05%, 0.5%, and 5% trans-cinnemaldehyde had significantly lower (p<0.0001 for each treatment group compared to water treated control) survival rates than aphids treated with water alone. These data indicate that there was a dose-dependent decrease in survival upon treatment with the natural antimicrobial trans-cinnemaldehyde.

### Trans-Cinnemaldehyde Treatment Decreased Buchnera in Aphids

To test whether different concentrations of trans-cinnemaldehyde, specifically resulted in loss of Buchnera in aphids, and that this loss impacted aphid fitness, DNA was extracted from aphids in each treatment group after 3 days of treatment and qPCR was performed to determine the Buchnera/aphid copy numbers. Aphids treated with water alone (control) had high ratios of Buchnera/aphid DNA copies. Similarly, aphids treated with the lowest concentration of trans-cinnemaldehyde (0.5%) had high ratios of Buchnera/aphid DNA copies.

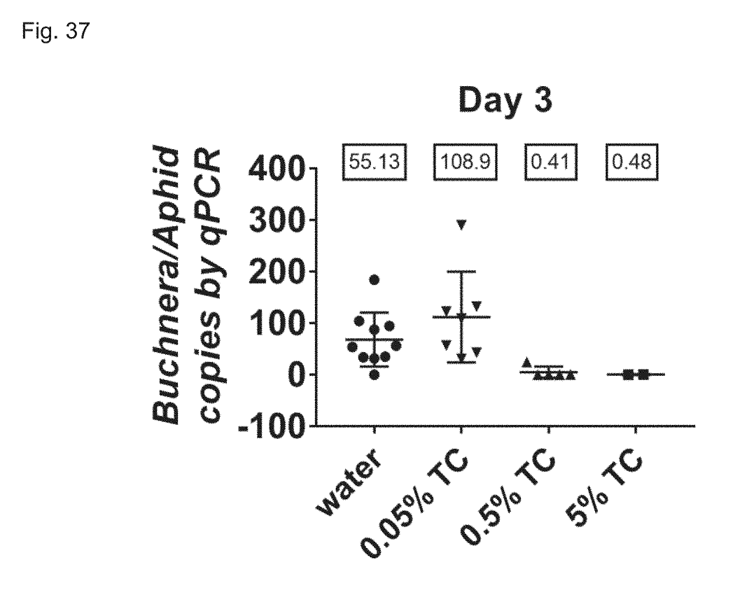

In contrast, aphids treated with 0.5 and 5% of trans-cinnemaldehyde had ˜870-fold less Buchnera/aphid DNA copies (FIG. 37), indicating that trans-cinnemaldehyde treatment decreased Buchnera levels, and that this decrease was concentration dependent.

Together this data described in the previous Examples demonstrate the ability to kill and decrease the development, reproductive ability, longevity, and endogenous bacterial populations, e.g., fitness, of aphids by treating them with plant secondary metabolite solution through multiple delivery methods.

### Example 17: Aphids Treated with Scorpion Antimicrobial Peptides

This Example demonstrates the treatment of aphids with multiple scorpion antimicrobial peptides (AMP), of which several are identified AMPs in the venom gland transcriptome of the scorpion Urodacus yaschenkoi (Luna-Ramirez et al., 2017, Toxins). AMPs typically have a net positive charge and hence, a high affinity for bacterial membranes. This Example demonstrates that the effect of the AMP on aphids was mediated through the modulation of bacterial populations endogenous to the aphid that were sensitive to AMP peptides. One targeted bacterial strain is Buchnera aphidicola, an obligate symbiont of aphids.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design:

The Uy192 solution was formulated using a combination of leaf perfusion and delivery through plants. The control solution was leaf injected with water+blue food coloring and water in tube. The treatment solution consisted of 100 ug/ml Uy192 in water via leaf injection (with blue food coloring) and through plant (in Eppendorf tube).

### Leaf Perfusion-Plant Delivery Experimental Design:

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 20±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 2 different treatment groups: 1) negative control (water treated), 2) The treatment solution of 100 ug/ml AMP in water. Each treatment group received approximately the same number of individuals from each of the collection plants.

Uy192 was synthesized by Bio-Synthesis at >75% purity. 1 mg of lyophilized peptide was reconstituted in 500 ul of 80% acetonitrile, 20% water, and 0.1% TFA, 100 ul (100 ug) was aliquoted into 10 individual Eppendorf tubes, and allowed to dry. For treatment (see Therapeutic design), 1 ml of water was added to a 100 ug aliquot of peptide to obtain the final concentration of Uy192 (100 ug/ml). The solution was then placed into a 1.5 ml Eppendorf tube with a fava bean stem with a leaf also perfused with the solutions and the opening of the tube was closed using parafilm. This feeding system was then placed into a deep petri dish (Fisher Scientific, Cat# FB0875711) and aphids were applied to the leaves of the plant.

For each treatment, 50 aphids were placed onto each leaf. The feeding systems were changed every 2-3 days throughout the experiment. Aphids were monitored daily for survival and dead aphids were removed from the deep petri dish when they were discovered.

In addition, the developmental stage (1^{st}, 2^{nd}, 3^{rd}, 4^{th}, 5^{th}, and 5R (5^{th} that has reproduced) instar) was determined daily throughout the experiment.

After 8 days of treatment, DNA was extracted from the remaining aphids in each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

There was a Negative Response on Insect Fitness Upon Treatment with the Scorpion AMPs

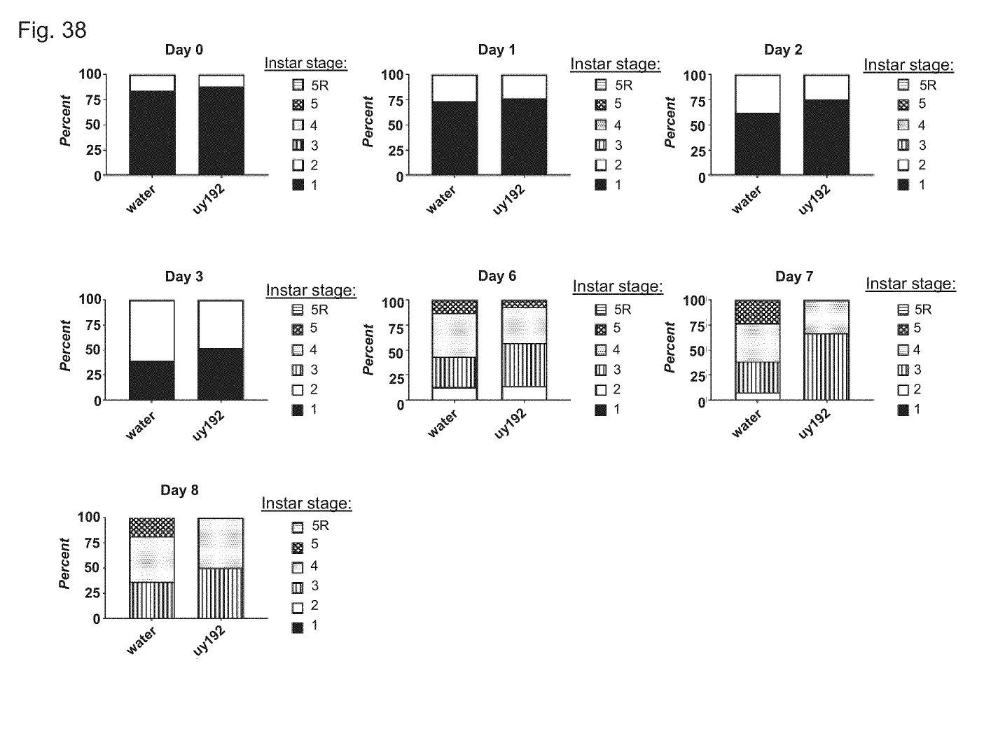

LSR-1 A. pisum 1^{st} and 2^{nd} instar aphids were divided into two separate treatment groups as defined in Experimental Design (above). Aphids were monitored daily and the number of aphids at each developmental stage was determined. Aphids treated with the negative control alone began reaching maturity (5^{th} instar stage) at approximately 6 days (FIG. 38). Development was delayed in aphids treated with Uy192, and after 8 days of treatment, aphids did not mature further than the 4^{rd} instar stage. These data indicate that treatment with Uy192 delayed and stopped progression of aphid development.

Treatment with Scorpion AMPs Results in Increased Aphid Mortality

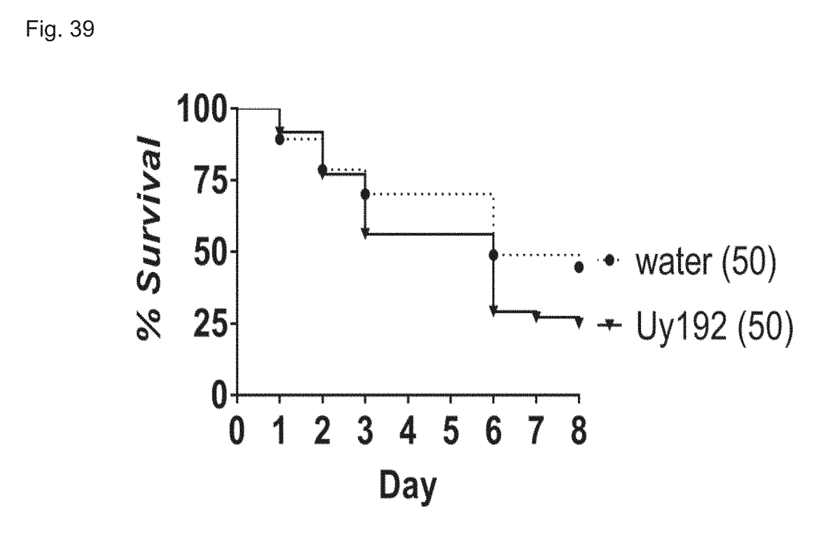

Survival rate of aphids was also measured during the treatments. The majority of the aphids treated with the control alone were alive at 3 days post-treatment (FIG. 39). After 4 days, aphids began gradually dying, and some survived beyond 7 days post-treatment.

In contrast, aphids treated with Uy192 had lower survival rates than aphids treated with control. These data indicate that there was a decrease in survival upon treatment with the scorpion AMP Uly192.

Treatment with Scorpion AMP Uy192 Results in Decreased Buchnera in Aphids

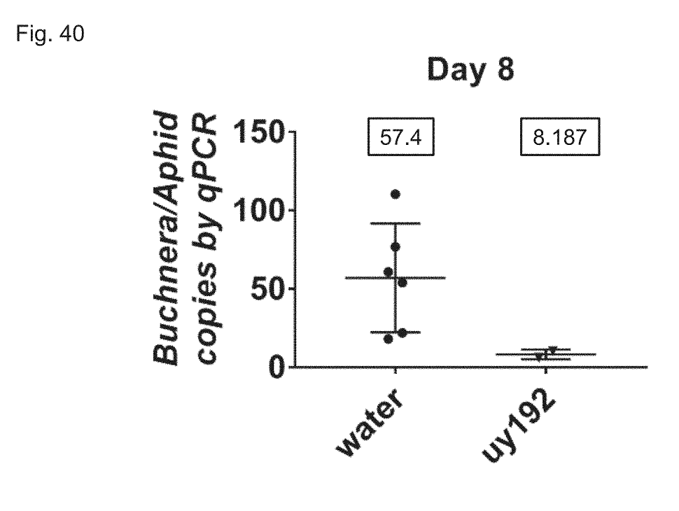

To test whether treatment with Uy192, specifically resulted in loss of Buchnera in aphids, and that this loss impacted aphid fitness, DNA was extracted from aphids in each treatment group after 8 days of treatment and qPCR was performed to determine the Buchnera/aphid copy numbers. Aphids treated with control alone had high ratios of Buchnera/aphid DNA copies. In contrast, aphids treated with 100 ug/ml Uy192 in water had ˜7-fold less Buchnera/aphid DNA copies (FIG. 40), indicating that Uy192 treatment decreased Buchnera levels.

Together this data described previously demonstrated the ability to kill and decrease the development, longevity, and endogenous bacterial populations, e.g., fitness, of aphids by treating them with a natural scorpion antimicrobial peptide.

### Example 18: Aphids Treated with Scorpion Antimicrobial Peptides

This Example demonstrates the treatment of aphids with several scorpion antimicrobial peptides (AMPs) D10, D3, Uyct3, and Uy17, which have been recently identified AMPs in the venom gland transcriptome of the scorpion Urodacus yaschenkoi (Luna-Ramirez et al., 2017, Toxins). AMPs typically have a net positive charge and hence, a high affinity for bacterial membranes. This Example demonstrates that the effect of the AMPs on aphids is mediated through the modulation of bacterial populations endogenous to the aphid that are sensitive to AMP peptides. One targeted bacterial strain is Buchnera aphidicola, an obligate symbiont of aphids.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design:

The indicated peptide or peptide cocktail (see Aphid Microinjection Experimental Design and Leaf perfusion-Plant Experimental Design sections for details below) was directly microinjected into aphids or delivered using a combination of leaf perfusion and delivery through plants. As a negative control, aphids were microinjected with water (for microinjection experiments) or placed on leaves injected with water and water in tube (for leaf perfusion and plant delivery experiments). The treatment solutions consisted of 20 nl of 5 μg/μl of D3 or D10 dissolved in water (for aphid microinjections) or 40 μg/ml of a cocktail of D10, Uy17, D3, and UyCt3 in water via leaf injection and through plant (in Eppendorf tube).

### Aphid Microinjection Experimental Design

Microinjection was performed using NanoJet III Auto-Nanoliter Injector with the in-house pulled borosilicate needle (Drummond Scientific; Cat#3-000-203-G/XL). Aphids (LSR-1 strain, Acyrthosiphon pisum) were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. Each treatment group had approximately the same number of individuals injected from each of the collection plants. Adult aphids were microinjected into the ventral thorax with 20 nl of either water or 100 ng (20 ul of a 5 ug/ml solution of peptide D3 or D10. The microinjection rate as 5 nl/sec. After injection, aphids were placed in a deep petri dish containing a fava bean leaf with stem in 2% agar.

Peptides were synthesized by Bio-Synthesis at >75% purity. 1 mg of lyophilized peptide was reconstituted in 500 μl of 80% acetonitrile, 20% water, and 0.1% TFA, 100 μl (100 μg) was aliquoted into 10 individual Eppendorf tubes, and allowed to dry.

After 5 days of treatment, DNA was extracted from the remaining aphids in each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

Microinjection of Aphids with Scorpion Peptides D3 and D10 Results in Decreased Insect Survival

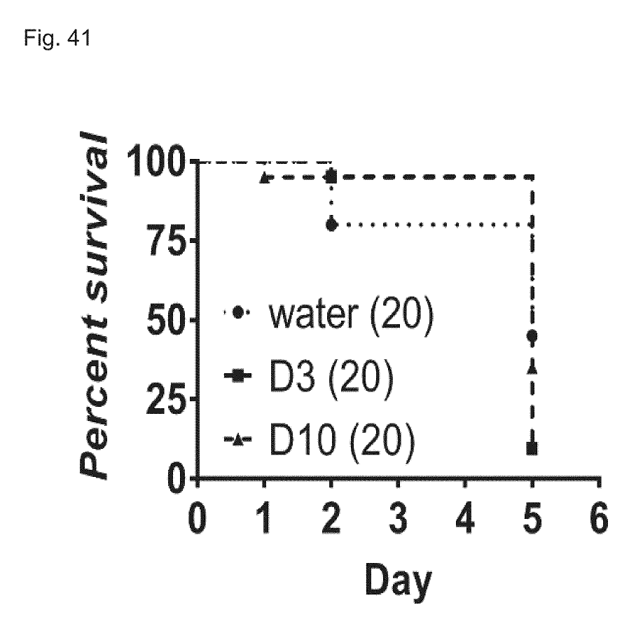

LSR-1 A. pisum 1^{st} and 2^{nd} instar aphids were divided into three separate treatment groups as defined in Experimental Design (described herein). Aphids were monitored daily and survival rate was determined. After 5 days of treatment, the aphids injected with the scorpion peptides had lower survival rates compared to water injected controls (9, 35, and 45% survival for injection with D3, D10, and water, respectively) (FIG. 41). These data indicate that there was a decrease in survival upon treatment with the scorpion AMPs D3 and D10.

Microinjection of Aphids with Scorpion Peptides D3 and D10 Results in a Reduction of Buchnera Endosymbionts

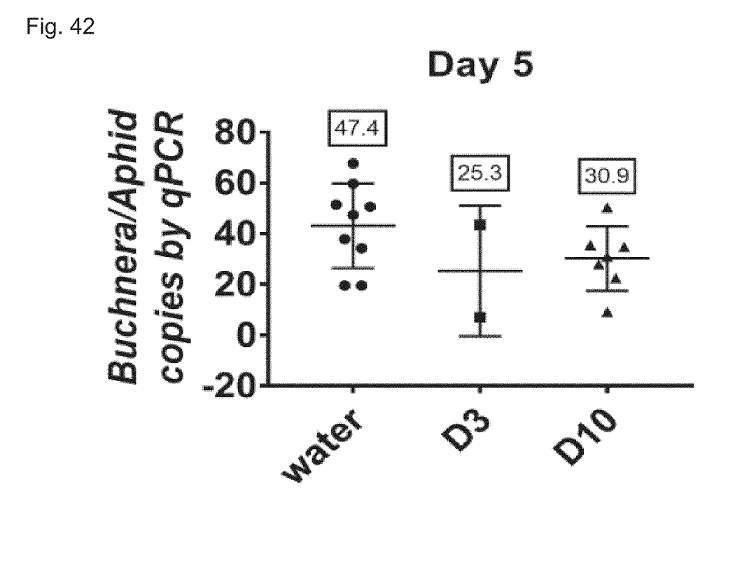

To test whether injection with the scorpion AMPs D3 and D10, specifically resulted in loss of Buchnera in aphids, and that this loss impacted aphid fitness, DNA was extracted from aphids in each treatment group 5 days after injection and qPCR was performed to determine the Buchnera/aphid copy numbers. Aphids injected with water alone had high ratios of Buchnera/aphid DNA (47.4) while aphids injected with D3 and D10 had lower ratios of Buchnera/aphid DNA (25.3 and 30.9, respectively) (FIG. 42). These data indicate that treatment with scorpion peptides D3 and D10 resulted in decreased levels of the bacterial symbiont Buchnera.

### Leaf Perfusion-Plant Delivery Experimental Design:

eNASCO Aphids (which harbor Buchnera and Serratia), Acyrthosiphon pisum were grown on fava bean plants (Vroma vicia faba from Johnny's Selected Seeds) as described above. For experiments, first and second instar aphids were collected from healthy plants and divided into 2 different treatment groups: 1) negative control (water treated), 2) The treatment solution consisted of 40 μg/ml of each D10, Uy17, D3, and UyCt3 in water. Each treatment group received approximately the same number of individuals from each of the collection plants.

Peptides were synthesized, dissolved, and aliquoted, as described above. For treatment (see Therapeutic design), water was added to a 100 μg aliquot of peptide to obtain the desired final concentration (40 μg/ml). The four peptides were combined to make the peptide cocktail solution. This solution was used to perfuse into leaves via injection. Following injection, the stems of the injected leaves were placed into a 1.5 ml Eppendorf tube which was then sealed with parafilm. This feeding system was then placed into a deep petri dish (Fisher Scientific, Cat# FB0875711) and aphids were applied to the leaves of the plant.

For each treatment, 41-49 aphids were placed onto each leaf. The feeding systems were changed every 2-3 days throughout the experiment. Aphids were monitored daily for survival and dead aphids were removed from the deep petri dish when they were discovered.

Treatment with Cocktail of Scorpion Peptides Results in Increased Aphid Mortality

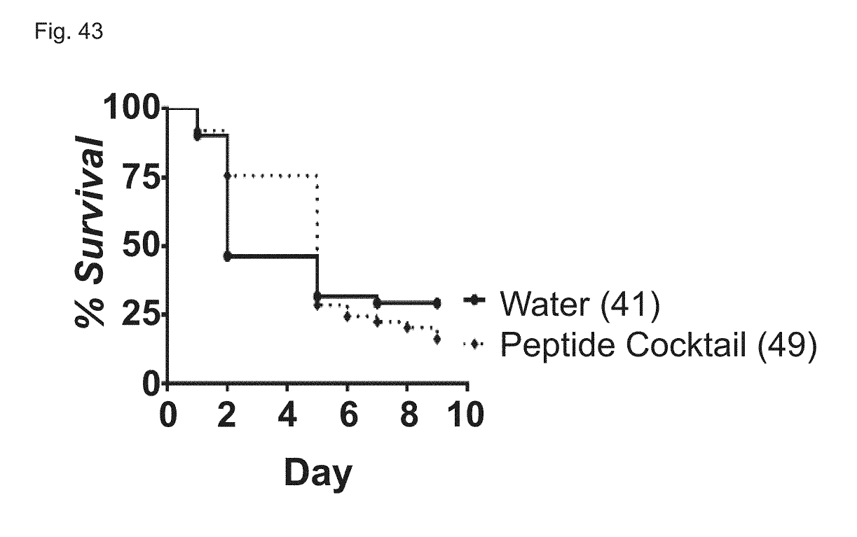

Survival rate of aphids was also measured during the treatments. After 6 days of treatment, aphids treated with the peptide cocktail had lower survival rates compared to those treated with water, and after 9 days the effect is more evident (16 and 29% survival for peptide cocktail and water treated, respectively) (FIG. 43). These data indicate that there was a decrease in survival upon treatment with the cocktail of scorpion AMPs, and as shown in previous Examples these AMP decreased the endosymbiont levels in the aphids.

Together this data described previously demonstrated the ability to kill and decrease the longevity and endogenous bacterial populations, e.g., fitness, of aphids by treating them with single natural scorpion antimicrobial peptides or a peptide cocktail.

### Example 19: Aphids Treated with an Antimicrobial Peptide Fused to a Cell Penetrating Peptide

This Example demonstrates the treatment of aphids with a fused scorpion antimicrobial peptide (AMP) (Uy192) to a cell penetrating peptide derived from a virus. The AMP Uy192 is one of several recently identified AMPs in the venom gland transcriptome of the scorpion Urodacus yaschenkoi (Luna-Ramirez et al., 2017, Toxins). AMPs typically have a net positive charge and hence, a high affinity for bacterial membranes. To enhance the delivery of the scorpion peptide Uy192 into aphid cells, the peptide was synthesized fused to a portion of the transactivator of transcription (TAT) protein of human immunodeficiency virus I (HIV-1). Previous studies have shown that conjugating this cell penetrating peptide (CPP) to other molecules increased their uptake into cells via transduction (Zhou et al., 2015 Journal of Insect Science and Cermenati et al., 2011 Journal of Insect Physiology). This Example demonstrates that the effect of the fused peptide on aphids was mediated through the modulation of bacterial populations endogenous to the aphid that were sensitive to the antimicrobial peptide. One targeted bacterial strain is Buchnera.

Aphids are agricultural insect pests causing direct feeding damage to the plant and serve as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design

The scorpion peptide conjugated to the cell penetrating peptide and fluorescently tagged with 6FAM (Uy192+CPP+FAM) was formulated using a combination of leaf perfusion and delivery through plants. The control solution (water) or treatment solution (Uy192+CPP+FAM) was injected into the leaf and placed in the Eppendorf tube. The treatment solution included 100 μg/ml Uy192+CPP+FAM in water.

### Leaf Perfusion-Plant Delivery Experimental Design

LSR-1 aphids, Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first instar aphids were collected from healthy plants and divided into 2 different treatment groups: 1) negative control (water treated), 2) Uy192+CPP+FAM treated with 100 μg/ml Uy192+CPP+FAM in water. Each treatment group received approximately the same number of individuals from each of the collection plants.

For treatment (see Therapeutic design), Uy192+CPP+FAM (amino acid sequence: YGRKKRRQRRRFLSTIWNGIKGLL-FAM) was synthesized by Bio-Synthesis at >75% purity. 5 mg of lyophilized peptide was reconstituted in 1 ml of 80% acetonitrile, 20% water, and 0.1% TFA, 50 μl (100 μg) was aliquoted into individual Eppendorf tubes, and allowed to dry. For treatment (see Therapeutic design), 1 ml of sterile water was added to a 100 μg aliquot of peptide to obtain the final concentration of Uy192+CPP+FAM (100 μg/ml). The solution was then injected into the leaf of the plant and the stem of the plant was placed into a 1.5 ml Eppendorf tube. The opening of the tube was closed using parafilm. This feeding system was then placed into a deep petri dish (Fisher Scientific, Cat# FB0875711) and aphids were applied to the leaves of the plant. Epi fluorescence imaging of the leaf confirmed that the leaves contained the green fluorescently tagged peptide in their vascular system.

For each treatment, 30 aphids were placed onto each leaf in triplicate. The feeding systems were changed every 2-3 days throughout the experiment. Aphids were monitored daily for survival and dead aphids were removed from the deep petri dish when they were discovered. In addition, the developmental stage (1st, 2nd, 3rd, 4th, 5th, and 5R (5th instar aphids that are reproducing) instar) was determined daily throughout the experiment.

At 5 days post-treatment, DNA was extracted from several aphids in each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

Treatment with Scorpion Peptide Uy192 Fused to a Cell Penetrating Peptide Delayed and Stopped Progression of Aphid Development

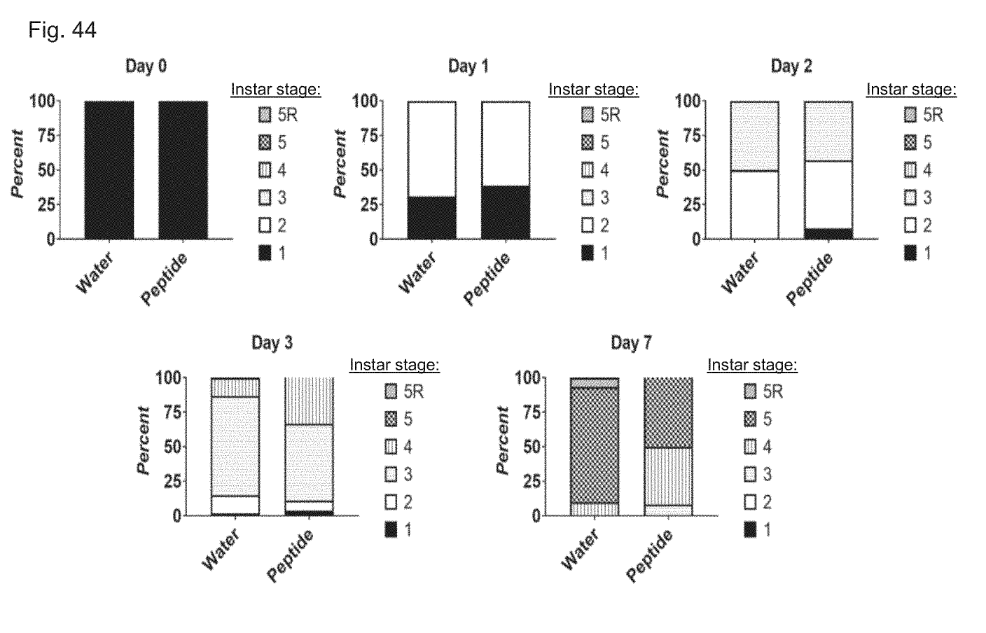

LSR-1 A. pisum 1st instar aphids were divided into three separate treatment groups as defined in Experimental Design (above). Aphids were monitored daily and the number of aphids at each developmental stage was determined. Development for both aphids treated with water and those treated with the scorpion peptide fused to the cell penetrating peptide was similar for days 0 and 1 (FIG. 44). By day 2, however, control treated aphids developed to either in the second or third instar stage, while some aphids treated with the scorpion peptide fused to the cell penetrating peptide remained in the first instar stage (FIG. 44). Even at 3 days post-treatment, some aphids treated with the scorpion peptide fused to the cell penetrating peptide remained in the first instar stage (FIG. 44). By 7 days post-treatment, the majority of the water treated aphids developed to the 5th or 5th reproducing instar stage. In contrast, only 50 percent of aphids treated with the scorpion peptide fused to the cell penetrating peptide developed to the 5th instar stage, while ˜42 and ˜8 percent of aphids remained at the 3rd or 4th instar stage, respectively (FIG. 44). These data indicate that treatment with the scorpion peptide Uy192 fused to the cell penetrating peptide delayed and stopped progression of aphid development.

Treatment with the Scorpion Peptide Uy192 Fused to a Cell Penetrating Peptide Resulted in Increased Aphid Mortality

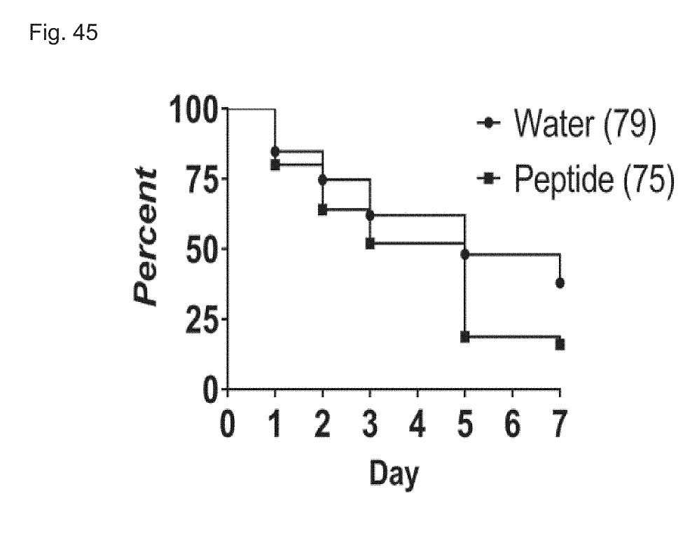

Survival rate of aphids was also measured during the treatments. Approximately 40% of aphids treated with the control alone survived the 7-day experiment (FIG. 45). In contrast, survival was significantly less for aphids treated with 100 μg/ml Uy192+CPP+FAM (p=0.0036, by Log-Rank Mantel Cox test), with only 16% of aphids surviving to day 7 (FIG. 45). These data indicate that there was a decrease in survival upon treatment with the scorpion peptide Uy192 fused to a cell penetrating peptide.

Treatment with a Scorpion Peptide Fused to a Cell Penetrating Peptide Resulted in Decreased Buchnera/Aphid DNA Ratios

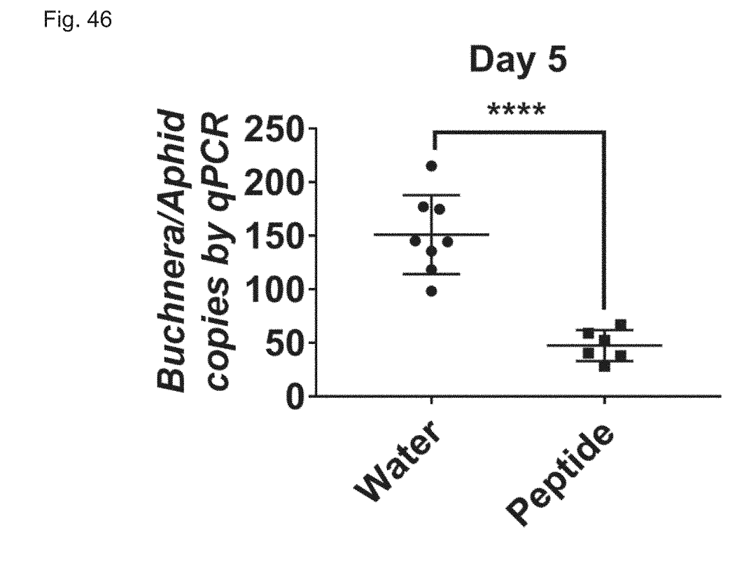

To test whether treatment with treatment with Uy192+CPP+FAM, specifically resulted in loss of Buchnera in aphids, and that this loss impacted aphid fitness, DNA was extracted from aphids in each group after 5 days of treatment, and qPCR was performed to determine the Buchnera/aphid copy numbers. Aphids treated with water had high ratios (˜134) of Buchnera/aphid DNA. In contrast, aphids treated with the scorpion peptide fused to the cell penetrating peptide had ˜1.8-fold less Buchnera/aphid DNA copies after 5 days of treatment (FIG. 46). These data indicate that treatment with the scorpion peptide fused to a cell penetrating peptide decreased endosymbiont levels.

### The Scorpion Peptide Fused to a Cell Penetrating Peptide Freely Entered the Bacteriocytes to Act Against Buchnera

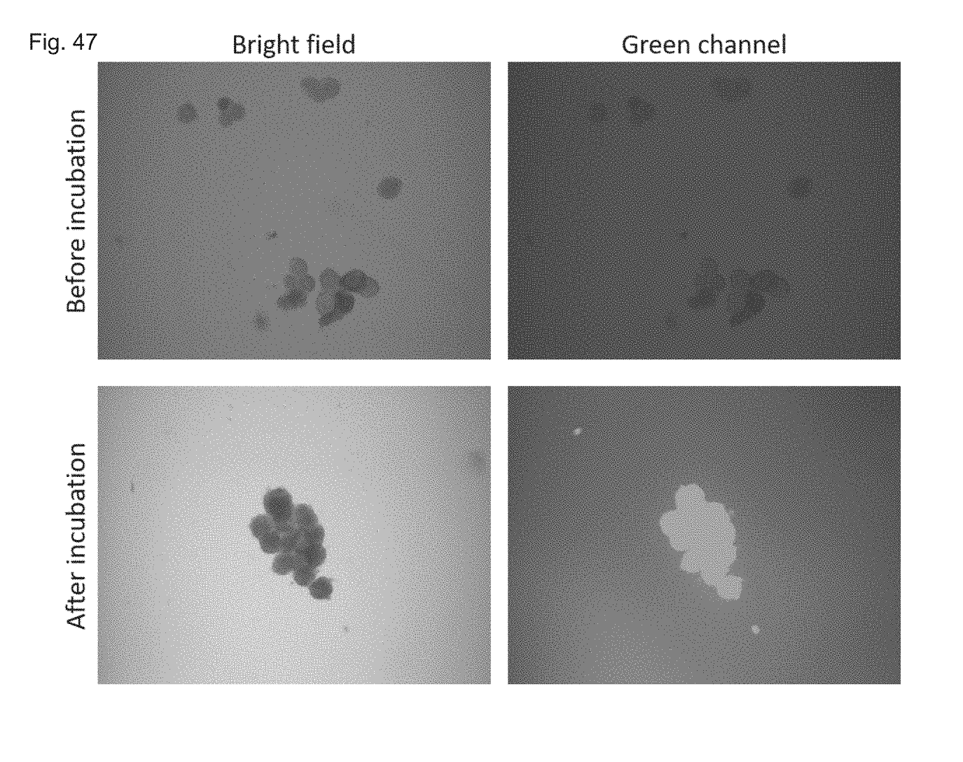

To test whether the cell penetrating peptide aids in the delivery of the scorpion peptide into the bacteriocytes directly, isolated bacteriocytes were directly exposed to the fusion protein and imaged. The bacteriocytes were dissected from the aphids in Schneider's medium supplemented with 1% w/v BSA (Schneider-BSA medium), and placed in an imaging well containing 20 ul of schneider's medium. A 100 ug lyophilized aliquot of the scorpion peptide was resuspended in 100 ul of the schneider's medium to produce 1 mg/ml solution, and 5 ul of this solution was mixed in to the well containing bacteriocytes. After 30 min of incubation at room temperature, the bacteriocytes were thoroughly washed to eliminate any excess free peptide in the solution. Images of the bacteriocytes were captured before and after the incubation (FIG. 47). The fusion peptide penetrated the bacteriocyte membranes and was directly available to Buchnera.

Together, this data demonstrates the ability to kill and decrease the development, longevity, and endogenous bacterial populations, e.g., fitness, of aphids by treating them with the scorpion antimicrobial peptide Uy192 fused to a cell penetrating peptide.

### Example 20: Aphids Treated with Vitamin Analogs

This Example demonstrates the treatment of aphids with the provitamin pantothenol which is the alcohol analog of pantothenic acid (Vitamin B5). Aphids have obligate endosymbiont bacteria, Buchnera, that help them make essential amino acids and vitamins, including Vitamin B5. A previous study has shown that pantothenol inhibits the growth of Plasmodium falciparium by inhibition of the specific parasite kinases (Saliba et al., 2005). It was hypothesized that treating aphids with pantothenol would be detrimental for the bacterial-insect symbiosis therefore affecting aphid fitness. This Example demonstrates that the treatment with pantothenol decreases aphid fitness.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

Therapeutic Design:

Pantothenol solutions were formulated depending on the delivery method:

1) In artificial diet through the plants: with 0 (negative control) or 10 or 100 uM pantothenol formulated in an artificial diet (based on Akey and Beck, 1971; see Experimental Design) without essential amino acids (2 mg/ml histidine, 2 mg/ml isoleucine, 2 mg/ml leucine, 2 mg/ml lysine, 1 mg/ml methionine, 1.62 mg/ml phenylalanine, 2 mg/ml threonine, 1 mg/ml tryptophan, and 2 mg/ml valine).

2) Leaf coating: 100 μl of 0.025% nonionic organosilicone surfactant solvent Silwet L-77 in water (negative control) or 100 μl of 50 μg/ml of rifampicin formulated in solvent solution was applied directly to the leaf surface and allowed to dry.

### Plant Delivery Experimental Design

Aphids (eNASCO, Acyrthosiphon pisum) were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first and second instar aphids were collected from healthy plants and divided into 3 different treatment groups: 1) artificial diet alone without essential amino acids, 2) artificial diet alone without essential amino acids and 10 uM pantothenol, and 3) artificial diet alone without essential amino acids and 100 uM pantothenol. Each treatment group received approximately the same number of individuals from each of the collection plants.

The artificial diet used was made as previously published (Akey and Beck, 1971 Continuous Rearing of the Pea Aphid, Acyrthosiphon pisum, on a Holidic Diet), with and without the essential amino acids (2 mg/ml histidine, 2 mg/ml isoleucine, 2 mg/ml leucine, 2 mg/ml lysine, 1 mg/ml methionine, 1.62 mg/ml phenylalanine, 2 mg/ml threonine, 1 mg/ml tryptophan, and 2 mg/ml valine), except neither diet included homoserine or beta-alanyltyrosine. The pH of the diets was adjusted to 7.5 with KOH and diets were filter sterilized through a 0.22 μm filter and stored at 4° C. for short term (<7 days) or at −80° C. for long term.

Pantothenol (Sigma Cat#295787) solutions were made at 10 uM and 100 uM in artificial diet without essential amino acids, sterilized by passing through a 0.22 μm syringe filter, and stored at −20° C. For treatments (see Therapeutic design), the appropriate amount of stock solution was added to the artificial diet without essential amino acids to obtain a final concentration of 10 or 100 uM pantothenol. The diet was then placed into a 1.5 ml Eppendorf tube with a fava bean stem with a leaf and the opening of the tube was closed using parafilm. This artificial diet feeding system was then placed into a deep petri dish (Fisher Scientific, Cat# FB0875711) and aphids were applied to the leaves of the plant.

For each treatment, 16-20 aphids were placed onto each leaf. Artificial diet feeding systems were changed every 2-3 days throughout the experiment. Aphids were monitored daily for survival and dead aphids were removed from the deep petri dish housing the artificial feeding system when they were discovered.

In addition, the developmental stage (1st, 2nd, 3rd, 4th, 5th instar) was determined daily throughout the experiment. Once an aphid reached the 4th instar stage, they were given their own artificial feeding system in a deep petri dish so that fecundity could be monitored once they reached adulthood.

For adult aphids, new nymphs were counted daily and then discarded. At the end of the experiments, fecundity was determined as the mean number of offspring produced daily once the aphid reached adulthood. Pictures of aphids were taken throughout the experiment to evaluate size differences between treatment groups.

After 8 days of treatment, DNA was extracted from multiple aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

### Vitamin Analog Treatment Delays Aphid Development

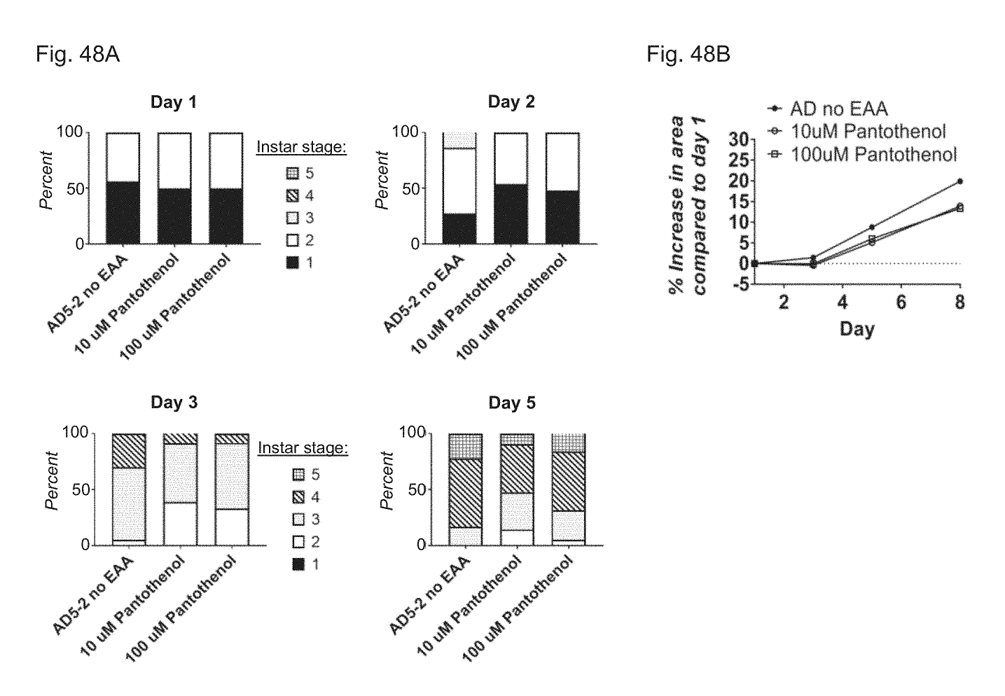

eNASCO 1st and 2nd instar aphids were divided into three separate treatment groups as defined in Plant Delivery Experimental Design (described herein). Aphids were monitored daily and the number of aphids at each developmental stage was determined. Aphids treated with artificial diet alone without essential amino acids began reaching maturity (5th instar stage) at approximately 5 days (FIG. 48A). Development was delayed in aphids treated with pantothenol, especially at days two and three post-treatment (FIG. 48A), indicating that treatment with pantothenol impairs aphid development. Eventually, most aphids from each treatment group reached maturity and began reproducing. In addition to monitoring developmental stage of aphids over time, aphids were also imaged and aphid area was determined. All aphids were the same size after 1 day of treatment, however, by 3 days post-treatment, aphids treated with pantothenol were smaller in area than untreated controls. Moreover, aphids treated with pantothenol had much less of an increase in body size (as determined by area) over the course of the experiment, compared to aphids treated with artificial diet alone without essential amino acids (FIG. 48B).

### Vitamin Analog Treatment Increased Aphid Mortality

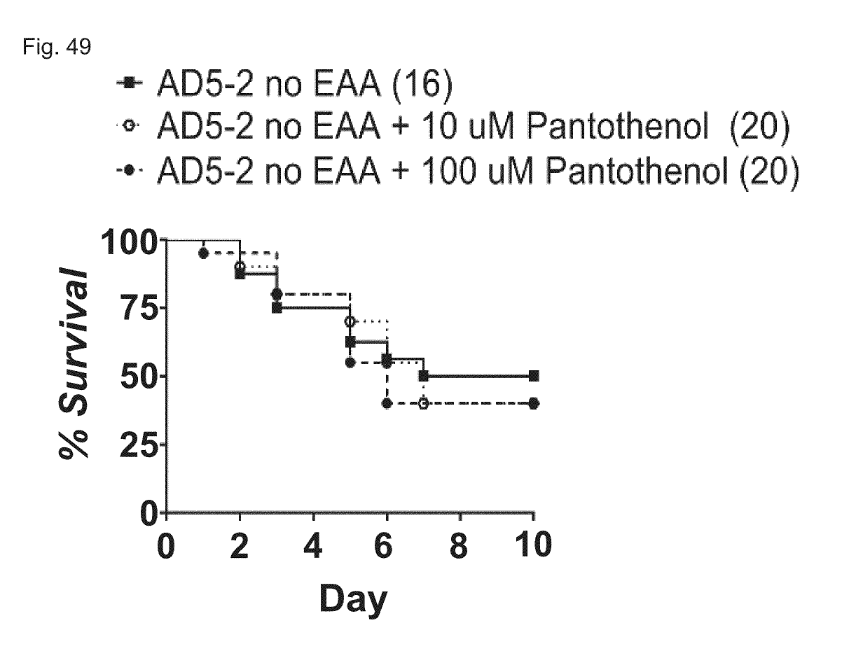

Survival rate of aphids was also measured during the treatments. Aphids reared on artificial diet alone without essential amino acids had higher survival rates compared to aphids treated with 10 or 100 uM pantothenol (FIG. 49), indicating that pantothenol treatment negatively affected aphid fitness.

Treatment with Pantothenol Decreases Aphid Fecundity

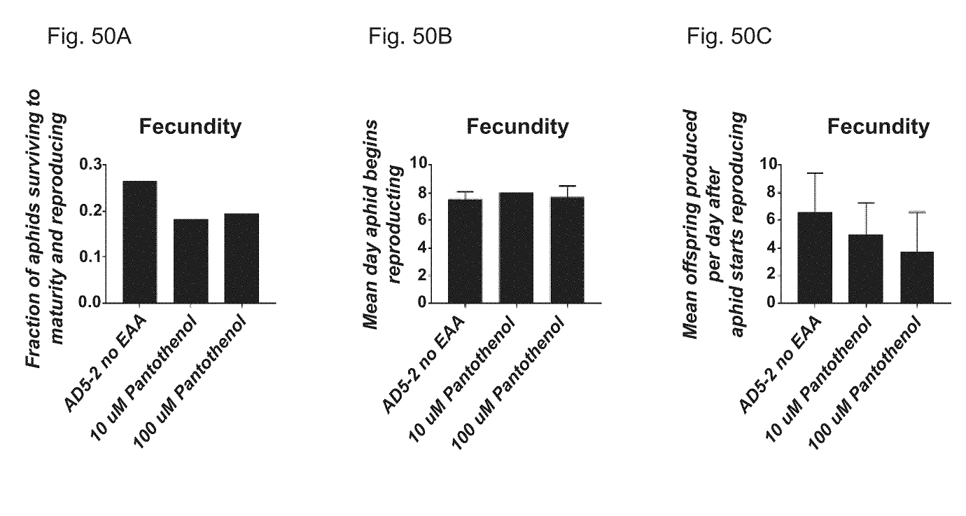

Fecundity was also monitored in aphids during the treatments. The fraction of aphids surviving to maturity and reproducing was determined. Approximately one quarter of aphids treated with artificial diet without essential amino acids survived to reach maturity by 8 days post-treatment (FIG. 50A). In contrast, only a little over 1/10th of aphids treated with 10 or 100 uM pantothenol survived to reach maturity and reproduce by 8 days post-treatment. The mean day aphids in each treatment group began reproducing was also measured and for all treatment groups, the mean day aphids began reproducing was 7 days (FIG. 50B). Additionally, the mean number of offspring per day produced by mature, reproducing aphids was also monitored. Aphids treated with artificial diet alone without essential amino acids produced approximately 7 offspring/day. In contrast, aphids treated with 10 and 100 uM pantothenol only produced approximately 4 and 5 offspring/day, respectively, shown in FIG. 50C. Taken together, these data indicate that pantothenol treatment resulted in a loss of aphid reproduction.

Pantothenol Treatment does not Affect Buchnera in Aphids

To test whether treatment with pantothenol, specifically resulted in loss of Buchnera in aphids, and that this loss impacted aphid fitness, DNA was extracted from aphids in each treatment group after 8 days of treatment and qPCR was performed to determine the Buchnera/aphid copy numbers. Aphids treated with artificial diet alone without essential amino acids had high ratios of Buchnera/aphid DNA copies as did aphids treated with each of the two concentrations of pantothenol (FIG. 51). These data indicate that pantothenol treatment does not affect Buchnera/aphid DNA copy number directly.

### Leaf Coating Delivery Experimental Design:

Pantothenol powder was added to 0.025% of a nonionic organosilicone surfactant solvent, Silwet L-77, to obtain a final concentration of 10 uM pantothenol. The treatment was filter sterilized using a 0.22 um filter and stored at 4 degrees C. Aphids (eNASCO strain, Acyrthosiphon pisum) were grown on fava bean plants as described in a previous Example. For experiments, first instar aphids were collected from healthy plants and divided into 2 different treatment groups: 1) negative control (solvent solution only) and 2) 10 uM pantothenol in solvent. 100 ul of the solution was absorbed onto a 2×2 cm piece of fava bean leaf.

Each treatment group received approximately the same number of individuals from each of the collection plant. For each treatment, 20 aphids were placed onto each leaf. Aphids were monitored daily for survival and dead aphids were removed when they were discovered. In addition, the developmental stage (1st, 2nd, 3rd, 4th, 5th instar, and 5R, representing a reproducing 5th instar) was determined daily throughout the experiment.

Pantothenol Treatment Delivered Through Leaf Coating does not Affect Aphid Development

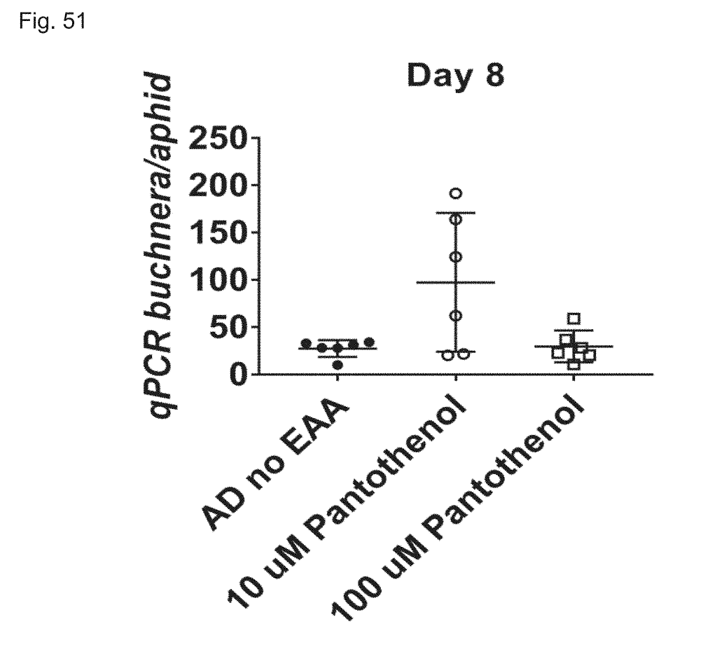

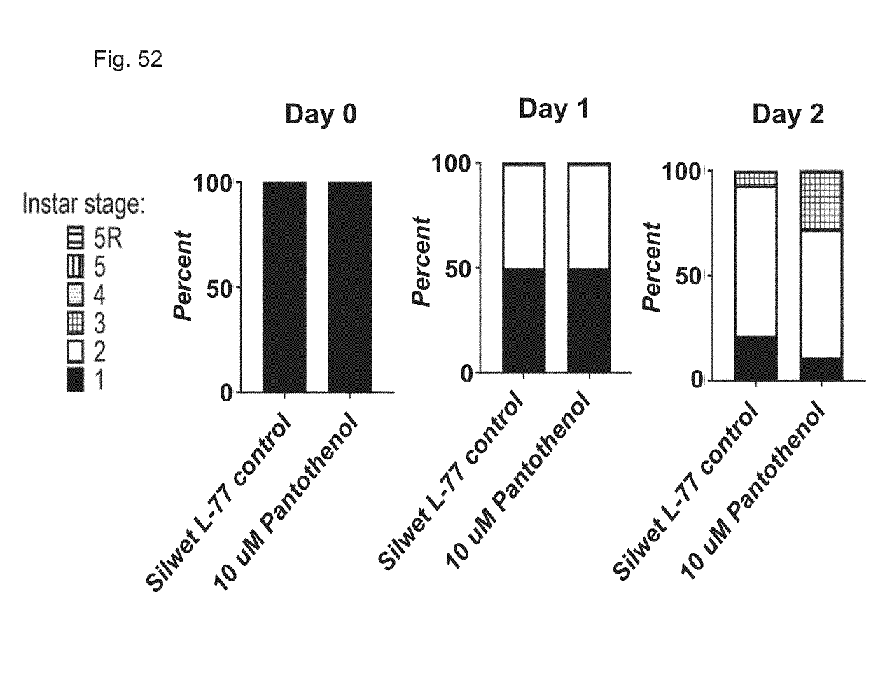

eNASCO 1st instar aphids were divided into two separate treatment groups as defined in the Experimental Design described herein. Aphids were monitored daily and the number of aphids at each developmental stage was determined. Aphids placed on coated leaves treated with either the control or pantothenol solution matured at similar rates up to two days post-treatment (FIG. 52). These data indicate that leaf coating with pantothenol did not affect aphid development.

### Pantothenol Treatment Delivered Through Leaf Coating Increased Aphid Mortality

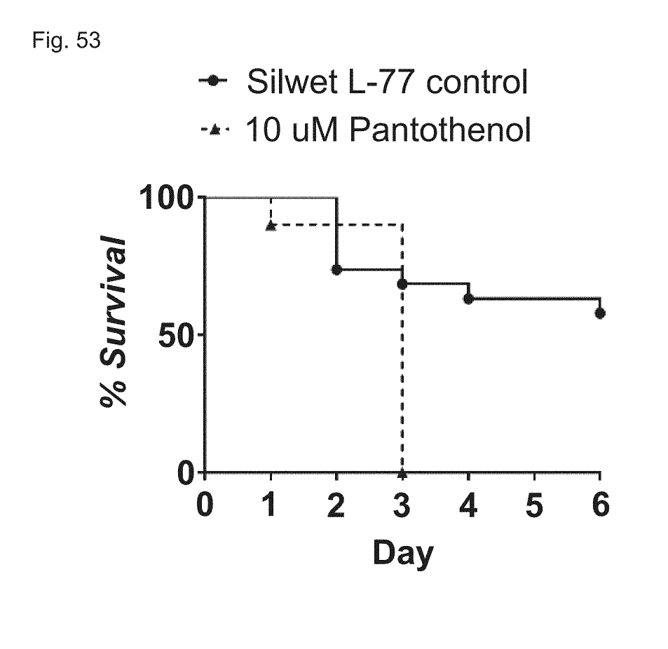

Survival rate of aphids was also measured during the leaf coating treatments. Aphids placed on coated leaves with pantothenol had lower survival rates than aphids placed on coated leaves with the control solution (FIG. 53). These data indicate that pantothenol treatment delivered through leaf coating significantly (p=0.0019) affected aphid survival. All aphids died in this experiment and there were no remaining aphids left to extract DNA from and determine Buchnera/aphid DNA ratios.

Together this data described in the previous Examples demonstrate the ability to kill and decrease the development, reproductive ability, longevity, and endogenous bacterial populations, e.g., fitness, of aphids by treating them with pantothenol through multiple delivery methods.

### Example 21: Aphids Treated with a Cocktail of Amino Acid Transporters Inhibitors

This Example demonstrates the treatment of aphids with a cocktail of amino acid analogs. The objective of this treatment was to inhibit uptakes of glutamine into the bacteriocytes through the ApGLNT1 glutamine transporter. It has previously been shown that arginine inhibits glutamine uptake by the glutamine transporter (Price et al., 2014 PNAS), and it was hypothesized that treatment with analogs of arginine, or other amino acid analogs, would also inhibit uptake of essential amino acids into the aphid bacteriocytes. This Example demonstrates that the decrease in fitness upon treatment was mediated through the modulation of bacterial populations endogenous to the aphid that were sensitive to amino acid analogs. One targeted bacterial strain is Buchnera.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design:

The amino acid cocktail was formulated for delivery through leaf perfusion and through the plant. This delivery method consisted of injecting leaves with approximately 1 ml of the amino acid cocktail in water (see below for list of components in the cocktail) or 1 ml of the negative control solution containing water only.

### Leaf Perfusion and Delivery Through Plants Experimental Design:

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first instar aphids were collected from healthy plants and divided into 2 different treatment groups: 1) negative control (water treatment) and 2) amino acid cocktail treatment. The amino acid cocktail contained each of the following agents at the indicated final concentrations: 330 μM L-NNA (N-nitro-L-Arginine; Sigma), 0.1 mg/ml L-canavanine (Sigma), 0.5 mg/ml D-arginine (Sigma), 0.5 mg/ml D-phenylalanine (Sigma), 0.5 mg/ml D-histidine (Sigma), 0.5 mg/ml D-tryptophan (Sigma), 0.5 mg/ml D-threonine (Sigma), 0.5 mg/ml D-valine (Sigma), 0.5 mg/ml D-methionine (Sigma), 0.5 mg/ml D-leucine, and 6 μM L-NMMA (citrate) (Cayman Chemical). ˜1 ml of the treatment solution was perfused into the fava bean leaf via injection and the stem of the plant was put into a 1.5 ml Eppendorf tube containing the treatment solution. The opening of the tube was closed using parafilm. This feeding system was then placed into a deep petri dish (Fisher Scientific, Cat# FB0875711) and aphids were applied to the leaves of the plant. For each treatment, a total of 56-58 aphids were placed onto each leaf (each treatment consisted of two replicates of 28-31 aphids). Each treatment group received approximately the same number of individuals from each of the collection plants. The feeding systems were changed every 2-3 days throughout the experiment. Aphids were monitored daily for survival and dead aphids were removed from the deep petri dish when they were discovered. The aphid developmental stage (1st, 2nd, 3rd, 4th, and 5th instar) was determined daily throughout the experiment and microscopic images were taken of the aphids on day 5 to determine aphid area measurements.

Stock solutions of L-NNA were made at 5 mM in water, sterilized by passing through a 0.22 μm syringe filter, and stored at −20° C. Stock solutions of L-canavanine were made at 100 mg/ml in water, sterilized by passing through a 0.22 μm syringe filter, and stored at 4° C. Stock solutions of D-arginine and D-threonine were made at 50 mg/ml in water, sterilized by passing through a 0.22 μm syringe filter, and stored at 4° C. Stock solutions of D-valine and D-methionine were made at 25 mg/ml in water, sterilized by passing through a 0.22 μm syringe filter, and stored at 4° C. Stock solutions of D-leucine were made at 12 mg/ml in water, sterilized by passing through a 0.22 μm syringe filter, and stored at 4° C. Stock solutions of D-phenylalanine and D-histidine were made at 50 mg/ml in 1M HCl, sterilized by passing through a 0.22 μm syringe filter, and stored at 4° C. Stock solutions of D-tryptophan were made at 50 mg/ml in 0.5M HCl, sterilized by passing through a 0.22 μm syringe filter, and stored at 4° C. Stock solutions of L-NMMA were made at 6 mg/ml in sterile PBS, sterilized by passing through a 0.22 μm syringe filter, and stored at −20° C. For treatments (see Therapeutic design), the appropriate amount of stock solution was added to water to obtain the final concentration of the agent in the cocktail as indicated above.

After 6 days of treatment, DNA was extracted from multiple aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

Treatment with a Cocktail of Amino Acid Analogs Delayed and Stopped Progression of Aphid Development

LSR-1 1st instar aphids were divided into two separate treatment groups as defined in Leaf perfusion and delivery through plants experimental design (described herein). Aphids were monitored daily and the number of aphids at each developmental stage was determined. Aphids treated with water began reaching maturity (5th instar stage) at day 5 post-treatment (FIG. 54A). By 6 days post-treatment, ˜20 percent of aphids treated with water reached the 5th instar stage. In contrast, less than 3 percent of the aphids treated with the amino acid cocktail reached the 5th instar stage, even after 6 days (FIG. 54A). This delay in development upon treatment with the amino acid cocktail was further exemplified by aphid size measurements taken at 5 days post-treatment. Aphids treated with water alone were approximately 0.45 mm2, whereas aphids treated with the amino acid cocktail were approximately 0.33 mm2 (FIG. 54B). These data indicate that treatment with the amino acid cocktail delayed aphid development, negatively impacting aphid fitness.

Treatment with an Amino Acid Analog Cocktail Resulted in Decreased Buchnera in Aphids

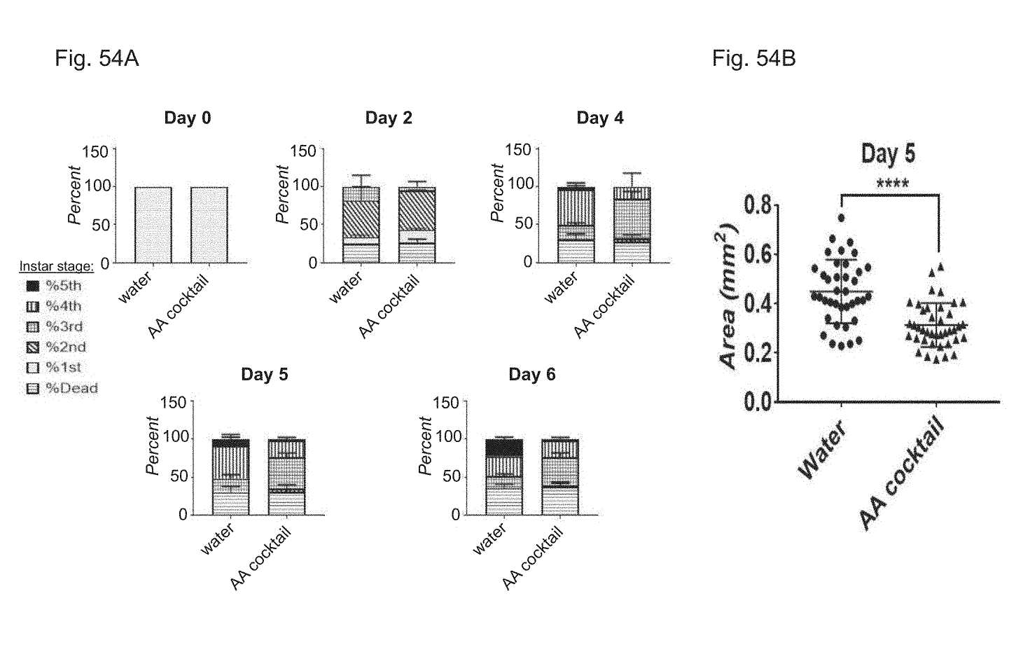

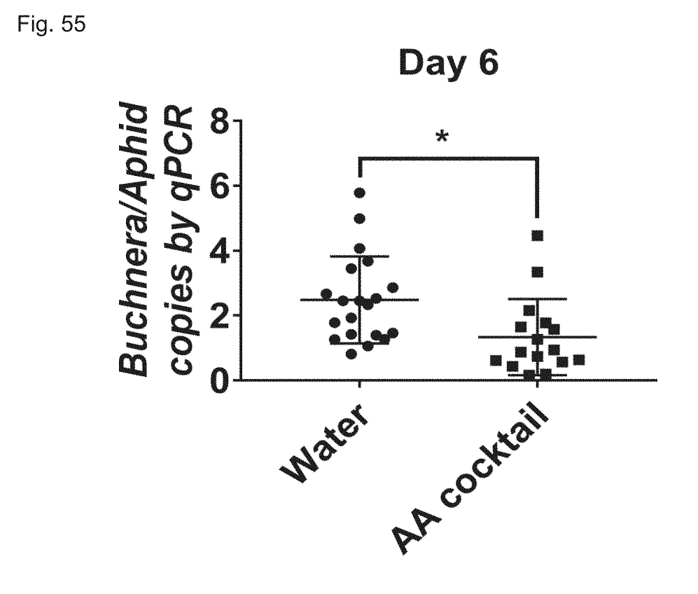

To test whether treatment with the amino acid analog cocktail specifically resulted in loss of Buchnera in aphids, and that this loss impacted aphid fitness, DNA was extracted from aphids in each treatment group after 6 days of treatment and qPCR was performed to determine the Buchnera/aphid copy numbers. Aphids placed on control solution had high ratios of Buchnera/aphid DNA copies. In contrast, aphids placed on AA cocktail treatment had a drastic reduction of Buchnera/aphid DNA copies (FIG. 55), indicating that the AA analog cocktail treatment eliminated endosymbiotic Buchnera.

Together, this data demonstrates the ability to decrease the development and endogenous bacterial populations, e.g., fitness, of aphids by treating them with a cocktail of amino acid analogs.

### Example 22: Aphids Treated with a Combination of Agents (Antibiotic, Peptide, and Natural Antimicrobial)

This Example demonstrates the treatment of aphids with a combination of three antimicrobial agents—an antibiotic (rifampicin), a peptide (the scorpion peptide Uy192), and a natural antimicrobial (low molecular weight chitosan). In other Examples, each of these agents administered individually resulted in decreased aphid fitness and reduced endosymbiont levels. This Example demonstrates that through the delivery of a combination of treatments, aphid fitness and endosymbiont levels were reduced as well as, or better than, treatment with each individual agent alone.

Aphids are agricultural insect pests causing direct feeding damage to the plant and serving as vectors of plant viruses. In addition, aphid honeydew promotes the growth of sooty mold and attracts nuisance ants. The use of chemical treatments, unfortunately still widespread, leads to the selection of resistant individuals whose eradication becomes increasingly difficult.

### Therapeutic Design

The combination treatment was formulated for delivery through leaf perfusion and through the plant. This delivery method consisted of injecting leaves with approximately 1 ml of the combination treatment in water (with final concentrations of 100 μg/ml rifampicin, 100 μg/ml Uy192, and 300 μg/ml chitosan) or 1 ml of the negative control solution containing water only.

### Leaf Perfusion and Delivery Through Plants Experimental Design

Aphids LSR-1 (which harbor only Buchnera), Acyrthosiphon pisum were grown on fava bean plants (Vroma vicia faba from Johnny's Selected Seeds) in a climate-controlled incubator (16 h light/8 h dark photoperiod; 60±5% RH; 25±2° C.). Prior to being used for aphid rearing, fava bean plants were grown in potting soil at 24° C. with 16 h of light and 8 h of darkness. To limit maternal effects or health differences between plants, 5-10 adults from different plants were distributed among 10 two-week-old plants, and allowed to multiply to high density for 5-7 days. For experiments, first instar aphids were collected from healthy plants and divided into 2 different treatment groups: 1) negative control (water treatment) and 2) a combination of 100 μg/ml rifampicin, 100 μg/ml Uy192, and 300 μg/ml chitosan treatment. 1 ml of the treatment solution was perfused into the fava bean leaf via injection and the stem of the plant was put into a 1.5 ml Eppendorf tube containing the treatment solution. The opening of the tube was closed using parafilm. This treatment system was then placed into a deep petri dish (Fisher Scientific, Cat# FB0875711) and aphids were applied to the leaves of the plant. For each treatment, a total of 56 aphids were placed onto each leaf (each treatment consisted of two replicates of 28 aphids). Each treatment group received approximately the same number of individuals from each of the collection plants. The feeding systems were changed every 2-3 days throughout the experiment. Aphids were monitored daily for survival and dead aphids were removed from the deep petri dish when they were discovered. The aphid developmental stage (1^{st}, 2^{nd}, 3^{rd}, 4^{th}, and 5^{th} instar) was determined daily throughout the experiment and microscopic images were taken of the aphids on day 5 to determine aphid area measurements.

Rifampicin (Tokyo Chemical Industry, LTD) stock solution was made at 25 mg/ml in methanol, sterilized by passing through a 0.22 μm syringe filter, and stored at −20° C. For treatment, the appropriate amount of stock solution was added to water to obtain a final concentration of 100 μg/ml rifampicin. Uy192 was synthesized by Bio-Synthesis at >75% purity. 1 mg of lyophilized peptide was reconstituted in 500 μl of 80% acetonitrile, 20% water, and 0.1% TFA. 100 μl (100 μg) was aliquoted into 10 individual Eppendorf tubes and allowed to dry. For treatment, 1 ml of water was added to a 100 μg aliquot of peptide to obtain the final concentration of 100 μg/ml Uy192. Chitosan (Sigma, catalog number 448869-50G) stock solution was made at 1% in acetic acid, sterilized autoclaving, and stored at 4° C. For treatments the appropriate amount of stock solution was added to water to obtain the final concentration of 300 μg/ml chitosan.

After 6 days of treatment, DNA was extracted from multiple aphids from each treatment group. Briefly, the aphid body surface was sterilized by dipping the aphid into a 6% bleach solution for approximately 5 seconds. Aphids were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and Buchnera and aphid DNA copy numbers were measured by qPCR. The primers used for Buchnera were Buch_groES_18F (CATGATCGTGTGCTTGTTAAG; SEQ ID NO: 228) and Buch_groES_98R (CTGTTCCTCGAGTCGATTTCC; SEQ ID NO: 229) (Chong and Moran, 2016 PNAS). The primers used for aphid were ApEF1a 107F (CTGATTGTGCCGTGCTTATTG; SEQ ID NO: 230) and ApEF1a 246R (TATGGTGGTTCAGTAGAGTCC; SEQ ID NO: 231) (Chong and Moran, 2016 PNAS). qPCR was performed using a qPCR amplification ramp of 1.6 degrees C./s and the following conditions: 1) 95° C. for 10 minutes, 2) 95° C. for 15 seconds, 3) 55° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

Treatment with a Combination of Three Antimicrobial Agents Delayed and Stopped Progression of Aphid Development

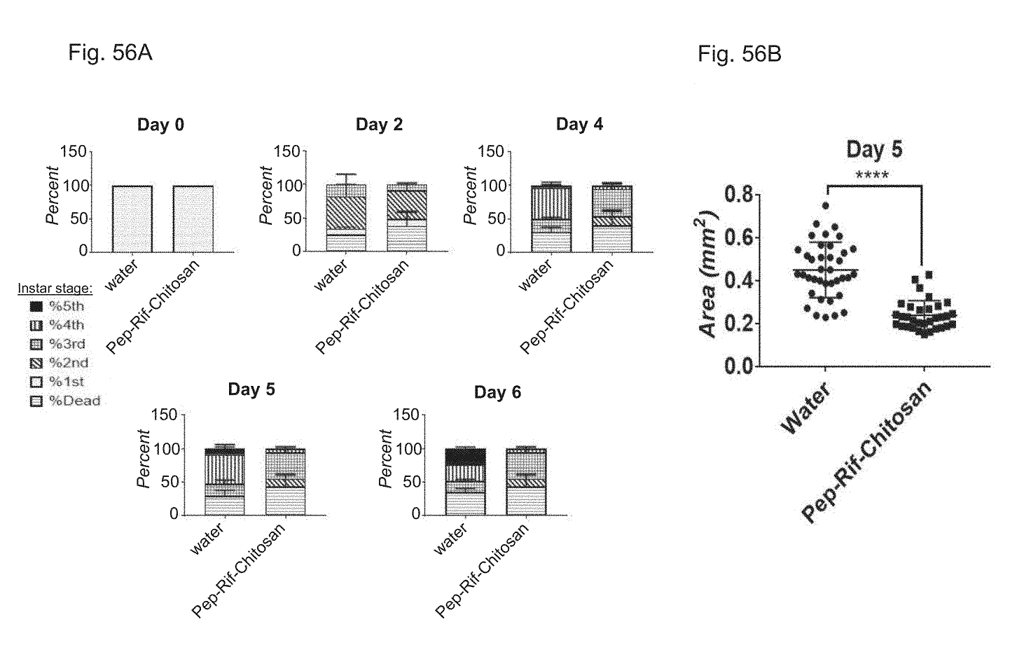

LSR-1 1^{st} instar aphids were divided into two separate treatment groups as defined in Leaf perfusion and delivery through plants experimental design (described herein). Aphids were monitored daily and the number of aphids at each developmental stage was determined. Aphids treated with water began reaching maturity (5^{th} instar stage) at day 5 post-treatment (FIG. 56A). By 6 days post-treatment, ˜20 percent of aphids treated with water reached the 5^{th} instar stage. In contrast, no aphids treated with the combination of three agents reached the 5^{th} instar stage, even after 6 days (FIG. 56A). This delay in development upon combination treatment was further exemplified by aphid size measurements taken at 5 days post-treatment. Aphids treated with water alone were approximately 0.45 mm², whereas aphids treated with the 3-agent combination were approximately 0.26 mm² (FIG. 56B). These data indicate that treatment with a combination of agents delayed aphid development, negatively impacting aphid fitness.

Treatment with a Combination of Three Antimicrobial Agents Increased Aphid Mortality

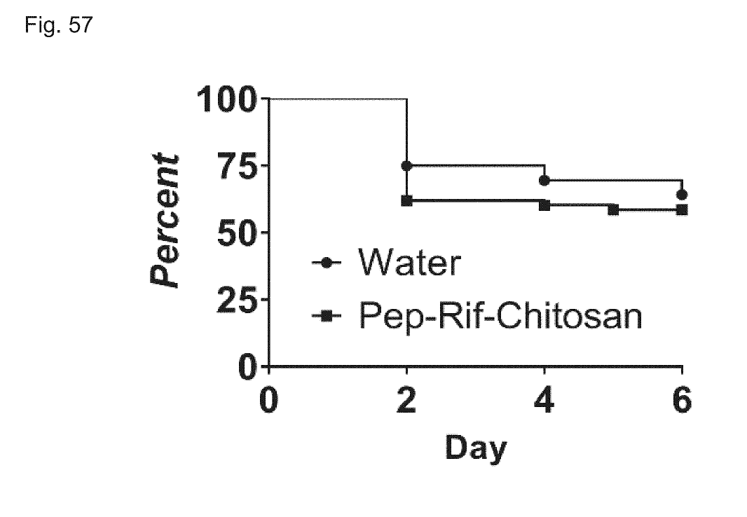

Survival was also monitored daily after treatment. At 2 days post-treatment, approximately 75 percent of aphids treated with water were alive, whereas only 62 percent of aphids treated with the combination of agents were alive. This trend of more aphids surviving treatment in the control (water-treated) group continued for the duration of the experiment. At 6 days post-treatment, 64 percent of control (water-treated) aphids survived, whereas 58 percent of aphids treated with a combination of rifampicin, Uy192, and chitosan survived (FIG. 57). These data indicate that the combination of treatments negatively affected aphid survival.

Treatment with a Combination of Three Agents Resulted in Decreased Buchnera in Aphids

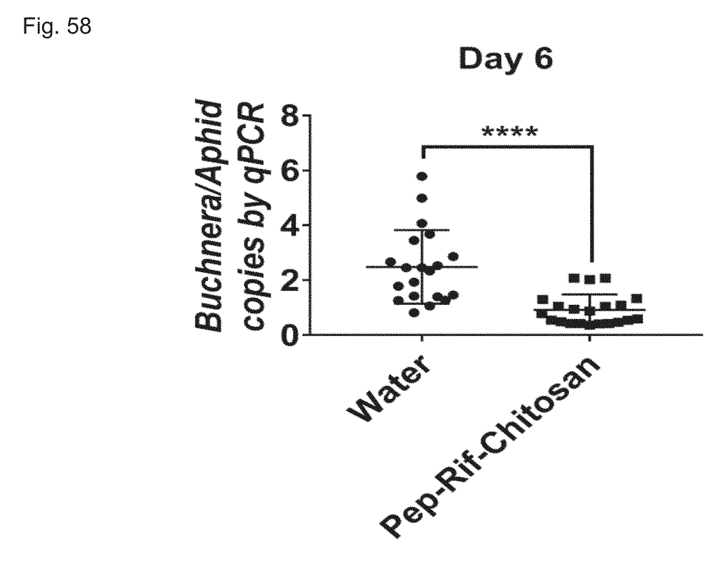

To test whether treatment with a combination of a peptide, antibiotic, and natural antimicrobial specifically resulted in loss of Buchnera in aphids, and that this loss impacted aphid fitness, DNA was extracted from aphids in each treatment group after 6 days of treatment and qPCR was performed to determine the Buchnera/aphid copy numbers. Aphids treated with water alone ratios of approximately 2.3 Buchnera/aphid DNA (FIG. 58). In contrast, aphids treated with the combination of a peptide, antibiotic, and natural antimicrobial had approximately 2-fold lower ratios of Buchnera/aphid DNA (FIG. 58). These data indicate that combination treatment reduced endosymbiont levels, which resulted in decreased aphid fitness.

Together, this data demonstrates the ability to decrease the development and endogenous bacterial populations, e.g., fitness, of aphids by treating them with a combination of a peptide, antibiotic, and natural antimicrobial.

### Example 23: Weevils Treated with an Antibiotic Solution

This Example demonstrates the effects of treatment of weevils with ciprofloxacin, a bactericidal antibiotic that inhibits the activity of DNA gyrase and topoisomerase, two enzymes essential for DNA replication. This Example demonstrates that the phenotypic effect of ciprofloxacin on weevils is mediated through the modulation of bacterial populations endogenous to the weevil that are sensitive to ciprofloxacin. One targeted bacterial strain is Sitophilus primary endosymbiont (SPE, Candidatus Sodalis pierantonius).

The genus Sitophilus comprises three weevil species known as storage pests (Sitophilus zemais, the maize weevil; Sitophilus oryzae, the rice weevil; and Sitophilus granarius, the grain weevil). All three of these weevil species harbor the intracellular symbiont, SPE, which provides the weevil with nutrients like vitamins and amino acids and improves mitochondrial energetic metabolism. Storage pests are controlled mainly by synthetic insecticides which leads to the selection of resistant individuals whose eradication becomes increasingly difficult.

### Experimental Design:

Sitophilus maize weevils (Sitophilus zeamais) were reared on organic corn at 27.5° C. and 70% relative humidity. Prior to being used for weevil rearing, corn was frozen for 7 days and then tempered to 10% humidity with sterile water. For experiments, adult male/female mating pairs were divided into 3 different treatment groups that were done in triplicate: 1) water control, 2) 250 μg/ml ciprofloxacin, and 3) 2.5 mg/ml ciprofloxacin. Ciprofloxacin (Sigma) stock solutions were made at 25 mg/ml in 0.1N HCl, sterilized by passing through a 0.22 μm syringe filter, and stored at −20° C. For treatments, the appropriate amount of stock solution was diluted in sterile water.

The weevils were subjected to three successive treatments:
1. The first treatment included soaking 25 g of corn with each of the three treatment groups listed above: 1) water control, 2) 250 μg/ml ciprofloxacin, and 3) 2.5 mg/ml ciprofloxacin. Briefly, 25 g of corn was placed into a 50 ml conical tube and each of the treatment was added to fill the tube completely. The tube was put on a shaker for 1.5 hours after which, the corn was removed and placed into a deep petri dish and air dried. Male/Female mating pairs were then added to each treatment group and allowed to feed for 4 days.
2. After 4 days, mating pairs were removed and subjected to a second treatment by putting them onto 25 g of new corn treated with 1) water control, 2) 250 μg/ml ciprofloxacin, and 3) 2.5 mg/ml ciprofloxacin. Mating pairs fed and laid eggs on this corn for 7 days. The corn from the second treatment was assessed for the emergence of offspring (see assessment of offspring, below)
3. Mating pairs were subjected to a final treatment which included a combination of submerging them into the treatment (1) water control, 2) 250 μg/ml ciprofloxacin, and 3) 2.5 mg/ml ciprofloxacin for 5 seconds and then placing them in a vial with 10 corn kernels that had been coated with 1 ml of 1) water control, 2) 250 μg/ml ciprofloxacin, and 3) 2.5 mg/ml ciprofloxacin.

Weevil survival was monitored daily for 18 days, after which DNA was extracted from the remaining weevils in each group. Briefly, the weevil body was surface sterilized by dipping the weevil into a 6% bleach solution for approximately 5 seconds. Weevils were then rinsed in sterile water and DNA was extracted from each individual aphid using a DNA extraction kit (Qiagen, DNeasy kit) according to manufacturer's instructions. DNA concentration was measured using a nanodrop nucleic acid quantification, and SPE and weevil DNA copy numbers were measured by qPCR. The primers used for SPE were qPCR_Sod_F (ATAGCTGTCCAGACGCTTCG; SEQ ID NO: 238) and qPCR_Sod_R (ATGTCGTCGAGGCGATTACC; SEQ ID NO: 239). The primers used for weevil (β-actin) were SACT144_FOR (GGTGTTGGCGTACAAGTCCT; SEQ ID NO: 240) and SACT314_REV (GAATTGCCTGATGGACAGGT; SEQ ID NO: 241) (Login et al., 2011). qPCR was performed using a qPCR amplification ramp of 1.6 degrees C./s and the following conditions: 1) 95° C. for 10 minutes, 2) 95° C. for 15 seconds, 3) 57° C. for 30 seconds, 4) repeat steps 2-3 40×, 5) 95° C. for 15 seconds, 6) 55° C. for 1 minute, 7) ramp change to 0.15 degrees C./s, 8) 95° C. for 1 second. qPCR data was analyzed using analytic (Thermo Fisher Scientific, QuantStudio Design and Analysis) software.

### Assessment of Offspring:

After 25 days, one replicate of the corn kernels from the second treatment of the adult mating pairs was dissected (see Experimental Design, above) to check for the presence of any developing larvae, pupae, or adult weevils. Most of the development of Sitophilus weevils takes place within the grain/rice/corn and adults emerge from the kernels once their development is complete. Corn kernels were gently dissected open with a scalpel and any developing weevils were collected and the percent of adults, pupae, and larvae were determined. The weevils from the dissection were then surface sterilized and the levels of SPE were determined by qPCR. Corn kernels from the remaining two replicates of each of the groups from the second treatment were not dissected but checked daily for the emergence of adult weevils.

Assessment of Antibiotic Penetration into Corn

25 mg of corn kernels was placed into a 50 ml conical tube and water or 2.5 mg/ml or 0.25 mg/ml ciprofloxacin in water was added to fill the tube. The kernels were soaked for 1.5 hours as described herein. After soaking, kernels were air dried and assayed to determine whether the antibiotic was able to coat and penetrate the kernel. To test this, a concentrated sample of Escherichia coli DH5α in water was spread onto 5 Luria Broth (LB) plates. To each plate the following was done, 1) a corn kernel soaked in water was added, 2) an entire corn kernel that had been soaked with 2.5 or 0.25 mg/ml ciprofloxacin was added, and 3) a half of corn kernel that had been soaked with 2.5 or 0.25 mg/ml ciprofloxacin was added and placed inside down on the plate. The plates were incubated overnight at 37 degrees C. and bacterial growth and/or zone(s) of inhibition were assessed the next day.

### Soaking Corn Kernels in Antibiotics Allowed Antibiotics to Coat the Surface and Penetrate Corn Kernels.

To test whether ciprofloxacin could coat the surface of a corn kernel after a kernel, corn kernels were soaked in water without antibiotics or water with 2.5 or 0.25 mg/ml ciprofloxacin (as described above). A concentrated culture of E. coli was then spread onto LB plates and one of the coated kernels was then placed onto the center of the plate. The plates were incubated overnight, and bacterial growth was assessed the next day.

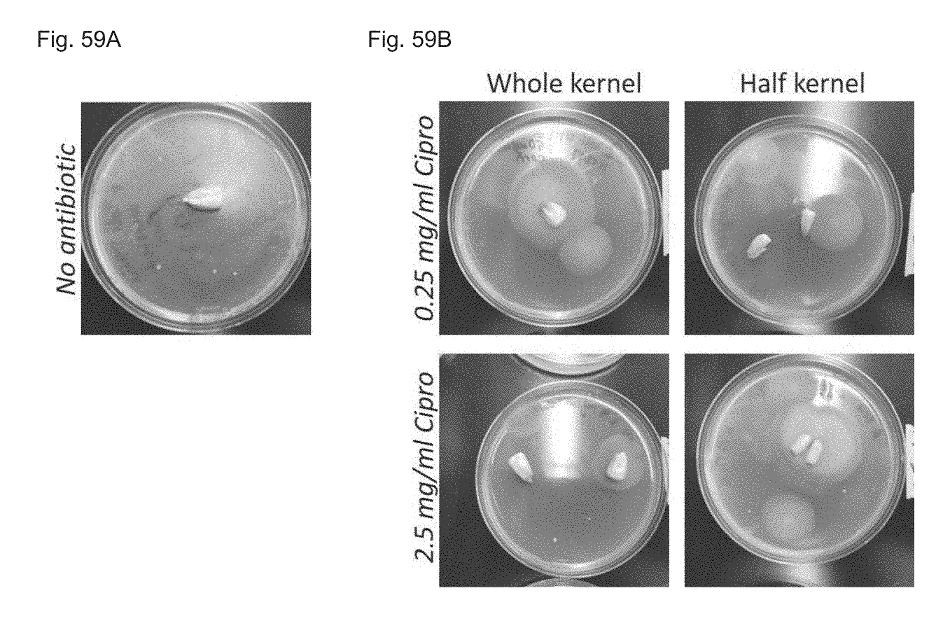

A lawn of bacteria grew on the entire plate with the corn kernel that had been coated in water without any antibiotics (FIG. 59A). In contrast, no bacteria grew on plates with entire corn kernels that had been soaked in either of the two concentrations of ciprofloxacin (FIG. 59B, left panels). These data show that the coating method employed in these experiments allowed for ciprofloxacin to successfully coat the surface of corn kernels and inhibit bacterial growth.

To test whether ciprofloxacin could penetrate the corn kernel, corn kernels soaked in 2.5 or 0.25 mg/ml ciprofloxacin were cut in half and placed cut side down on an LB plate with a concentrated culture of E. coli. The plates were incubated overnight and the next day bacterial growth was assessed. No bacterial growth was present on the plates with the kernels soaked in either concentration of antibiotic, indicating that ciprofloxacin penetrated the corn kernel (FIG. 59B, right panels). Together, these data indicate that the method of corn kernel soaking used for these experiments successfully coated and penetrated the kernels with the antibiotic.

### Antibiotic Treatment Decreases SPE Levels in the F0 Generation.

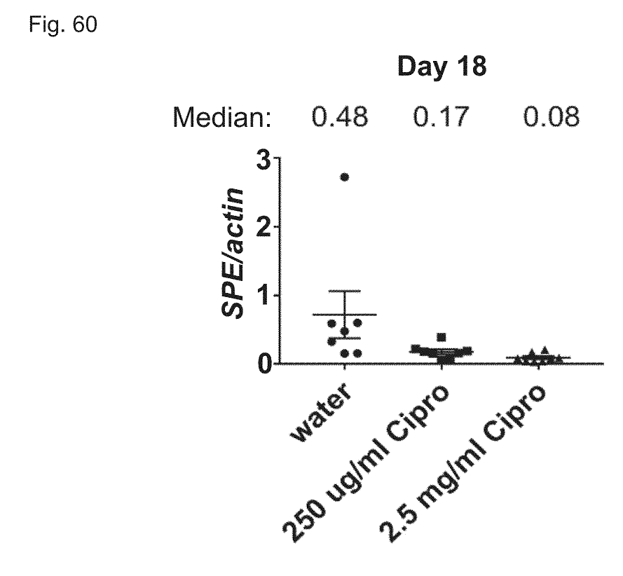

S. zeamais mating pairs were divided into three separate treatment groups as defined in Experimental Design (above). Weevils were monitored daily and all weevils remained alive for the course of the experiment. After 18 days of treatment, weevils were surface sterilized, genomic DNA was extracted, and SPE levels were measured by qPCR. Weevils treated with water only had approximately 4 and 8-fold higher amounts of SPE compared to weevils treated with 250 ug/ml and 2.5 mg/ml ciprofloxacin, respectively (FIG. 60). These data indicate that treatment of weevils with ciprofloxacin resulted in decreased levels of SPE.

### Antibiotic Treatment Delays the Development and Decreases the SPE Levels of the F Generation of Weevils.

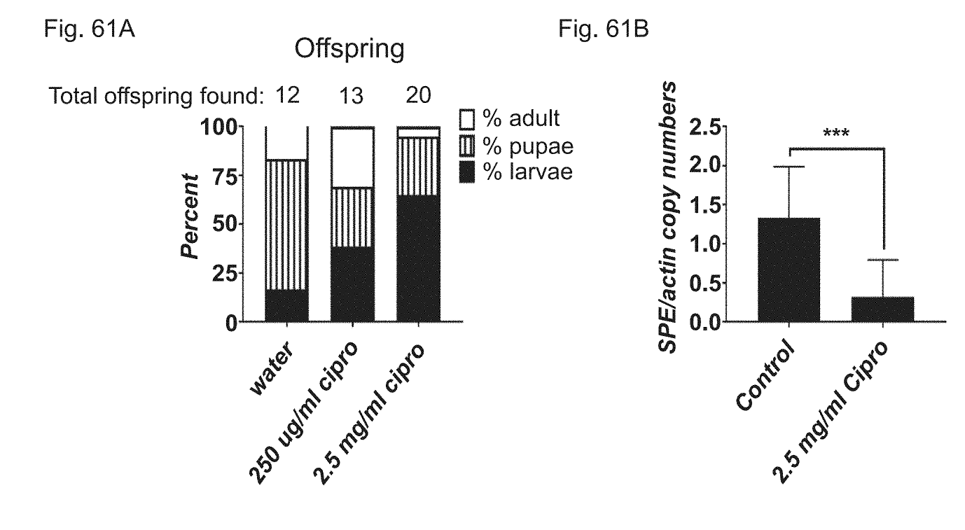

The development of the F1 generation of weevils was assessed by dissecting corn kernels that F0 mating pairs had oviposited on for 7 days and were subsequently removed. After 25 days, 12 offspring were found in water/control-treated corn with the majority (˜67%) of offspring being in the pupae form (FIG. 61A). 13 and 20 offspring were found in weevils treated with 250 ug/ml and 2.5 mg/ml ciprofloxacin, respectively. Interestingly, weevils treated with antibiotic showed a delay in development compared to control treated weevils with the majority (38 and 65% for 250 ug/ml and 2.5 mg/ml ciprofloxacin, respectively) of the offspring being in the larval form (FIG. 61A).

Genomic DNA was extracted from weevils dissected from the corn kernels and qPCR was performed to measure the levels of SPE. Water treated F1 weevils had approximately 4-fold higher levels of SPE compared to weevils treated with 2.5 mg/ml ciprofloxacin (FIG. 61B). These data indicate that treatment with ciprofloxacin reduced the levels of the SPE in weevils which led to a delay in development.

### Antibiotic Treatment Decreased Weevil Reproduction

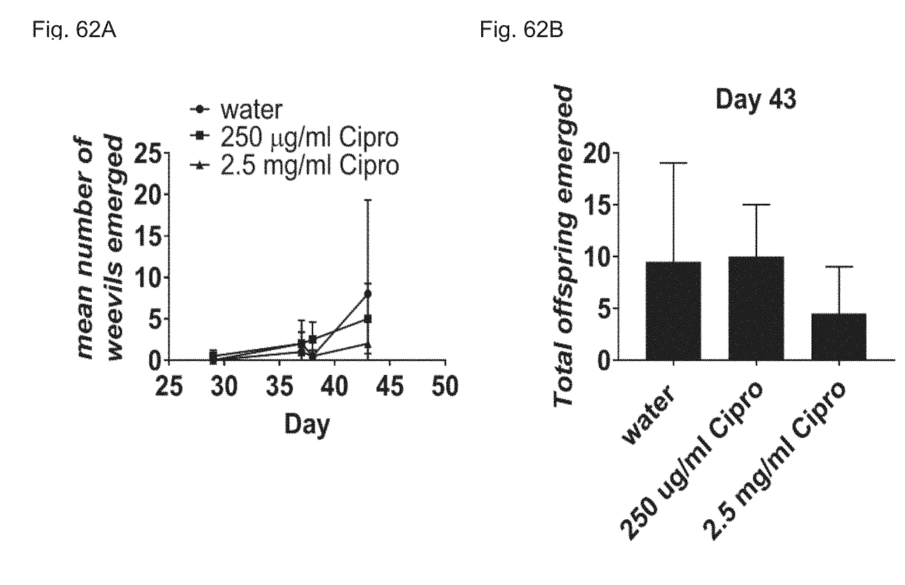

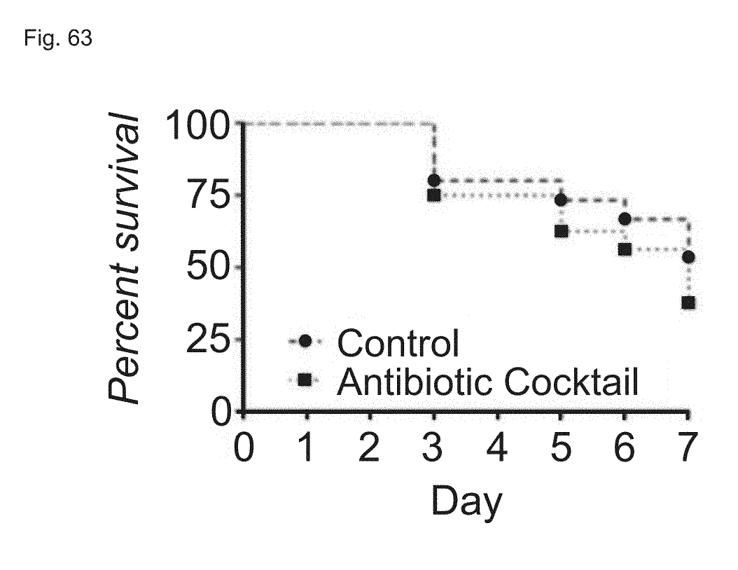

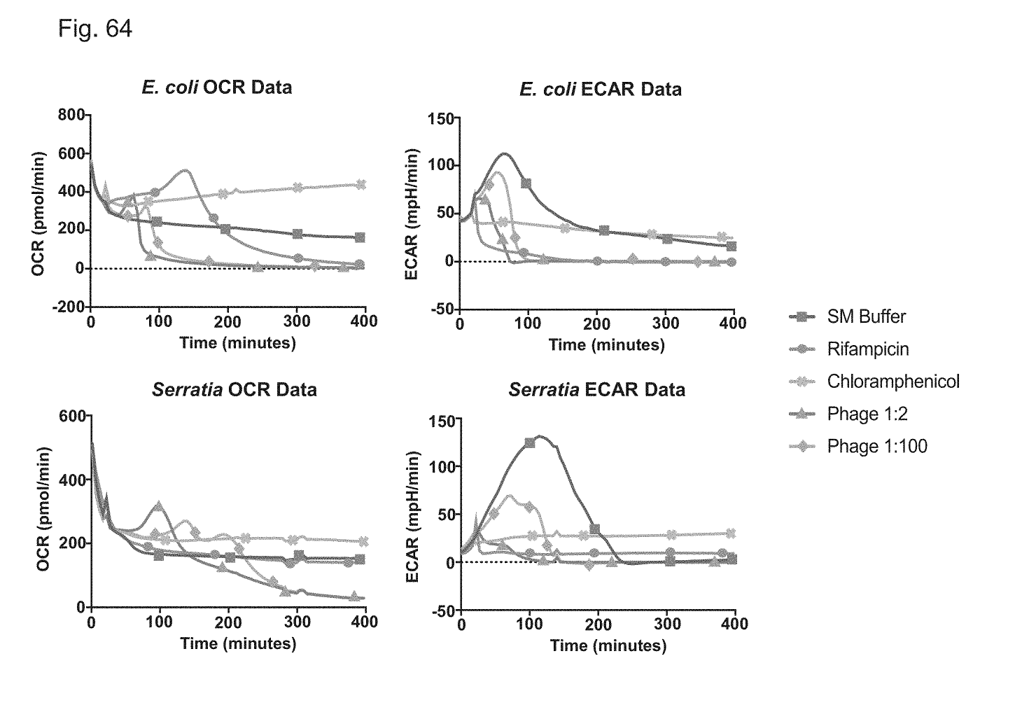

The number of weevils that emerged over the course of 43 days after the initial mating pairs were removed from the second treatment was used a measure for the fecundity FIGS. 62A and 62B). The first weevil emerged on day 29, and the total number of weevils that emerged till day 43 were counted. While weevils treated with water and 250 ug/ml had similar amount of F1 offspring, there were much less offspring that emerged from the 2.5 mg/ml treatment group, indicating that antibiotic treatment decreased SPE levels affected weevil fecundity.

Together with the previous Examples, this data demonstrate the ability to kill and decrease the development, reproductive ability, longevity, and endogenous bacterial populations, e.g., fitness, of weevils by treating them with an antibiotic through multiple delivery methods.

### Example 24: Mites Treated with an Antibiotic Solution

This Example demonstrates the ability to kill, decrease the fitness of two-spotted spider mites by treating them with rifampicin, a narrow spectrum antibiotic that inhibits DNA-dependent RNA synthesis by inhibiting a bacterial RNA polymerase, and doxycycline, a broad-spectrum antibiotic that prevents bacterial reproduction by inhibiting protein synthesis. The effect of rifampicin and doxycycline on mites was mediated through the modulation of bacterial populations endogenous to the mites that were sensitive to the antibiotics.

Insects, such as mosquitoes, and arachnids, such as ticks, can function as vectors for pathogens causing severe diseases in humans and animals such as Lyme disease, dengue, trypanosomiases, and malaria. Vector-borne diseases cause millions of human deaths every year. Also, vector-borne diseases that infect animals, such as livestock, represent a major global public health burden. Thus, there is a need for methods and compositions to control insects and arachnids that carry vector-borne diseases. Two-spotted spider mites are arachnids in the same subclass as ticks. Therefore, methods and compositions used to decrease the fitness of two-spotted spider mites can provide insight into decreasing tick fitness.

### Therapeutic Design

Two treatments were used for these experiments 1) 0.025% Silwet L-77 (negative control) or 2) a cocktail of antibiotics containing 250 μg/ml rifampicin and 500 μg/ml doxycycline. Rifampicin (Tokyo Chemical Industry, LTD) stock solutions were made at 25 mg/ml in methanol, sterilized by passing through a 0.22 μm syringe filter, and stored at −20° C. Doxcycline (manufacturer) stock solutions were made at 50 mg/mL in water, sterilized by passing through a 0.22 μm syringe filter, and stored at −20° C.

### Experimental Design:

This assay tested an antibiotic solution on two-spotted spider mites and determined how their fitness was altered by targeting endogenous microbes.

Kidney plants were grown in potting soil at 24° C. with 16 h of light and 8 h of darkness. Mites were reared on kidney bean plants at 26° C. and 15-20% relative humidity. For treatments, one-inch diameter leaf disks were cut from kidney bean leaves and sprayed with either 0.025% Silwet L-77 (negative control) or the antibiotic cocktail (250 μg/ml rifampicin and 500 μg/ml doxycycline in 0.025% Silwet L-77) using a Master Airbrush Brand Compressor Model C-16-B Black Mini Airbrush Air Compressor. The compressor was cleaned with ethanol before, after, and between treatments. The liquid was feed through the compressor using a quarter inch tube. A new tube was used for each treatment.

After leaf discs dried, four of each treatment were placed in a cup on top of a wet cotton ball covered with a piece of kimwipe. Each treatment setup was done in duplicate. 25 adult female mites were then placed in the cup. On day 4, the females were removed from the cup and the eggs and larvae were left on the leaf discs.

On day 11, mites at the protonymph stage and the deutonymph stage were taken from the cups and placed in their own tube so survival could be measured. Each tube contained a moist cotton ball covered with a piece of kimwipe with a half inch leaf disc treated with the negative control or the cocktail.

The mites were observed under a dissecting microscope daily after feeding on a leaf treated with the antibiotic or the control solutions, and classified according to the following categories:
Alive: they walked around when on their legs or moved after being poked by a paint brush.
Dead: immobile and did not react to stimulation from a paint brush A sterile paint brush was used to stimulate the mites by touching their legs. Mites classified as dead were kept throughout the assay and rechecked for movement daily. The assays were carried out at 26° C. and 15-20% relative humidity.

### Antibiotic Treatment Increased Mite Mortality

The survival rates of the two-spotted spider mites treated with the antibiotic cocktail were compared to the mites treated with the negative control. The survival rates of the mites treated with the cocktail were decreased compared to the control (FIG. 60).

This data demonstrates the ability to decrease fitness of mites by treating them with a solution of antibiotics.

### Example 25: Insects Treated with a Solution of Purified Phage

This Example demonstrates the isolation and purification of phages from environmental samples that targeted specific insect bacteria. This Example also demonstrates the efficacy of isolated phages against the target bacteria in vitro by plaque assays, by measuring their oxygen consumption rate, and the extracellular acidification rate. Finally, this Example demonstrates the efficacy of the phages in vivo, by measuring the ability of the phage to the target bacteria from flies by treating them with a phage isolated against the bacteria. This Example demonstrates that a pathogenic bacterium that decreased the fitness of an insect can be cleared using a phage to target the bacteria. Specifically, Serratia marcescens which is a pathogenic bacterium in flies can be cleared with the use of a phage that was isolated from garden compost.

### Experimental Design

Isolation of Specific Bacteriophages from Natural Samples:

Bacteriophages against target bacteria were isolated from environmental source material. Briefly, a saturated culture of Serratia marcescens was diluted into fresh double-strength tryptic soy broth (TSB) and grown for ˜120 minutes to early log-phase at 24-26° C., or into double-strength Luria-Bertani (LB) broth and grown for ˜90 min at 37° C. Garden compost was prepared by homogenization in PBS and sterilized by 0.2 μm filtration. Raw sewage was sterilized by 0.2 μm filtration. One volume of filtered source material was added to log-phase bacterial cultures and incubation was continued for 24 h. Enriched source material was prepared by pelleting cultures and filtering supernatant fluid through 0.45 μm membranes.

Phages were isolated by plating samples onto double-agar bacterial lawns. Stationary bacterial cultures were combined with molten 0.6% agar LB or TSB and poured onto 1.5% agar LB or TSB plates. After solidification, 2.5 μL of phage sample dilutions were spotted onto the double-agar plates and allowed to absorb. Plates were then wrapped and incubated overnight at 25° C. (TSA) or 37° C. (LB), then assessed for the formation of visible plaques. Newly isolated plaques were purified by serial passaging of individual plaques on the target strain by picking plaques into SM Buffer (50 mM Tris-HCl [pH 7.4], 10 mM MgSO4, 100 mM NaCl) and incubating for 15 min at 55° C., then repeating the double-agar spotting method from above using the plaque suspension.

Bacteriophages were successfully isolated from both sewage and compost, as detailed above. Plaque formation was clearly evident after spotting samples onto lawns of the S. marcescens bacteria used for the enrichments.

Passaging, Quantification, and Propagation of Bacteriophages:

Propagation and generation of phage lysates for use in subsequent experiments was performed using bacteriophages isolated and purified as above. Briefly, saturated bacterial cultures were diluted 100-fold into fresh medium and grown for 60-120 minutes to achieve an early-logarithmic growth state for effective phage infection. Phage suspensions or lysates were added to early log phase cultures and incubation was continued until broth clearing, indicative of phage propagation and bacterial lysis, was observed, or until up to 24 h post-infection. Lysates were harvested by pelleting cells at 7,197×g for 20 min, then filtering the supernatant fluid through 0.45 or 0.2 μm membranes. Filtered lysates were stored at 4° C.

Enumeration of infective phage particles was performed using the double-agar spotting method. Briefly, a 1:10 dilution series of samples was performed in PBS and dilutions were spotted onto solidified double-agar plates prepared with the host bacteria as above. Plaque-forming units (PFU) were counted after overnight incubation to determine the approximate titer of samples.

In Vitro Analysis of Isolated Phages Measuring Bacterial Respiration:

A Seahorse XFe96 Analyzer (Agilent) was used to measure the effects of phages on bacteria by monitoring oxygen consumption rate (OCR) and extracellular acidification rate (ECAR) during infection. XFe96 plates were coated the day prior to experiments by 15 μL of a 1 mg/mL poly-L-lysine stock per well and dried overnight at 28° C. and XFe96 probes were equilibrated by placing into wells containing 200 μL of XF Calibrant and incubating in the dark at room temperature. The following day, poly-L-lysine coated plates were washed twice with ddH2O. Saturated overnight cultures of E. coli BL21 (LB, 37° C.) or S. marcescens (TSB, 25° C.) were subcultured at 1:100 into the same media and grown with aeration for ˜2.5 h at 30° C. Cultures were then diluted to O.D.600 nm 0.02 using the same media. Treatments were prepared by diluting stocks into SM Buffer at 10× final concentration and loading 20 μL of the 10× solutions into the appropriate injection ports of the probe plate. While the probes were equilibrating in the XFe96 Flux Analyzer, bacterial plates were prepared by adding 90 μL of bacterial suspensions or media controls and spun at 3,000 rpm for 10 min. Following centrifugation, an additional 90 μL of the appropriate media were added gently to the wells so as not to disturb bacterial adherence, bringing the total volume to 180 μL per well.

The XFe96 Flux Analyzer was run at 30° C., following a Mix, Wait, Read cycling of 1:00, 0:30, 3:00. Four cycles were completed to permit equilibration/normalization of bacteria, then the 20 μL treatments were injected and cycling continued as above, for a total time of approximately 6 h. Data were analyzed using the Seahorse XFe96 Wave software package.

The effects of isolated bacteriophages were assayed by measuring oxygen consumption rate (OCR) and extracellular acidification rate (ECAR) of bacteria with a Seahorse XFe96 Analyzer. When E. coli was infected with phage T7 and S. marcescens infected with the newly isolated ϕSmVL-C1, dramatic decreases in OCR were observed following brief bursts in this rate (FIG. 64). For both phages with both host organisms, the Seahorse assay permitted the detection of successful phage infection without the need for plaque assays. Thus, this method is applicable for detecting phage infection of a host organism not amenable to traditional phage detection methods.

SYBR Gold Transduction Assay for Infection Identification:

Bacteriophage preparations were prepared for staining by pretreating with nucleases to remove extraviral nucleic acids that could interfere with fluorescent signal interpretation. Briefly, MgCl2 was added to 10 mL of phage lysate at 10 mM final concentration, and RNase A (Qiagen) and DNase I (Sigma) were both added to final concentrations of 10 μg/mL. Samples were incubated for 1 h at room temperature. After nuclease treatment, 5 mL of lysates were combined with 1 μL of SYBR Gold (Thermo, 10,000×) and incubated at room temperature for ˜1.5 h. Excess dye was subsequently removed from samples using Amicon ultrafiltration columns. Briefly, Amicon columns (15 mL, 10 k MWCO) were washed by adding 10 mL of SM Buffer and spinning at 5,000×g, 4° C. for 5 min. Labeled phage samples were then spun through the columns at 5,000×g, 4° C. until the volume had decreased by approximately 10-fold (15-30 min). To wash samples, 5 mL SM Buffer was added to each reservoir and the spin repeated, followed by two additional washes. After the third wash, the retained samples were pipetted out from the Amicon reservoirs and brought up to approximately 1 mL using SM Buffer. To remove larger contaminants, washed and labeled phage samples were spun at 10,000×g for 2 min, and the supernatants were subsequently filtered through 0.2 μm membranes into black microtubes and stored at 4° C.

Saturated bacterial cultures (E. coli MG1655 grown in LB at 37° C., S. marcescens and S. symbiotica grown in TSB at 26° C.) were prepared by spinning down 1 mL aliquots and washing once with 1 mL PBS before a final resuspension using 1 mL PBS. Positive control labeled bacteria were stained by combining 500 μL of washed bacteria with 1 μL of SYBR Gold and incubating for 1 h in the dark at room temperature. Bacteria were pelleted by spinning at 8,000×g for 5 min and washed twice with an equal volume of PBS, followed by resuspension in a final volume of 500 μL PBS. A volume of 25 μL of stained bacteria was combined with 25 μL of SM Buffer in a black microtube, to which 50 μL of 10% formalin (5% final volume, 2% formaldehyde) was added and mixed by flicking. Samples were fixed at room temperature for 3 h and then washed using Amicon ultrafiltration columns. Briefly, 500 μL of picopure water was added to Amicon columns (0.5 mL, 100 k MWCO) and spun at 14,000×g for 5 min to wash membranes. Fixed samples were diluted by adding 400 μL of PBS and then transferred to pre-washed spin columns and spun at 14,000×g for 10 min. Columns were transferred to fresh collection tubes, and 500 μL of PBS was added to dilute out fixative remaining in the retentate. Subsequently, two additional PBS dilutions were performed, for a total of three washes. The final retentates were diluted to roughly 100 μL, then columns were inverted into fresh collection tubes and spun at 1,000×g for 2 min to collect samples. Washed samples were transferred to black microtubes and stored at 4° C.

For transduction experiments and controls, 25 μL of bacteria (or PBS) and 25 μL of SYBR Gold labeled phage (or SM Buffer) were combined in black microtubes and incubated static for 15-20 min at room temperature to permit phage adsorption and injection into recipient bacteria. Immediately after incubation, 50 μL of 10% formalin was added to samples and fixation was performed at room temperature for ˜4 h. Samples were washed with PBS using Amicon columns, as above.

Injection of bacteriophage nucleic acid was required for a phage to successfully infect a host bacterial cell. Coliphage P1kc labeled with SYBR Gold and co-incubated with S. marcescens revealed the presence of fluorescent bacteria by microscopy, validating the use of this assay in a phage isolation pipeline. As with the Seahorse assay, this approach provided an alternative to traditional phage methods to permit expansion to organisms not amenable to plaque assay. Additionally, the SYBR Gold transduction assay did not require bacterial growth, so is applicable to analysis of phages targeting difficult or even non-culturable organisms, including endosymbionts such as Buchnera.

Testing In Vivo Efficacy of the Phages Against S. marcescens in Drosophila melanogaster Flies

S. marcescens cultures were grown in Tryptic Soy Broth (TSB) at 30° C. with constant shaking at 200 rpm.

The media used to rear fly stocks was cornmeal, molasses and yeast medium (11 g/l yeast, 54 g/l yellow cornmeal, 5 g/l agar, 66 ml/I molasses, and 4.8 ml/I propionic acid). All the components of the diet except propionic acid were heated together to 80° C. in deionized water with constant mixing for 30 minutes and let to cool to 60° C. Propionic acid was then mixed in and 50 ml of the diet was aliquoted into individual bottles and allowed to cool down and solidify. The flies were raised at 26° C., 16:8 hour light:dark cycle, at around 60% humidity.

To infect the flies with S. marcescens, a fine needle (About 10 um wide tip) was dipped in a dense overnight stationary phase culture and the thorax of the flies was punctured. For this experiment, four replicates of 10 males and 10 females each were infected with S. marcescens using the needle puncturing method as the positive control for fly mortality. For the treatment group, four replicates of 10 males and 10 females each were pricked with S. marcescens and a phage solution containing about 108 phage particles/ml. Finally, two replicates of 10 males and 10 females each that were not pricked or treated in anyway were used as a negative control for mortality.

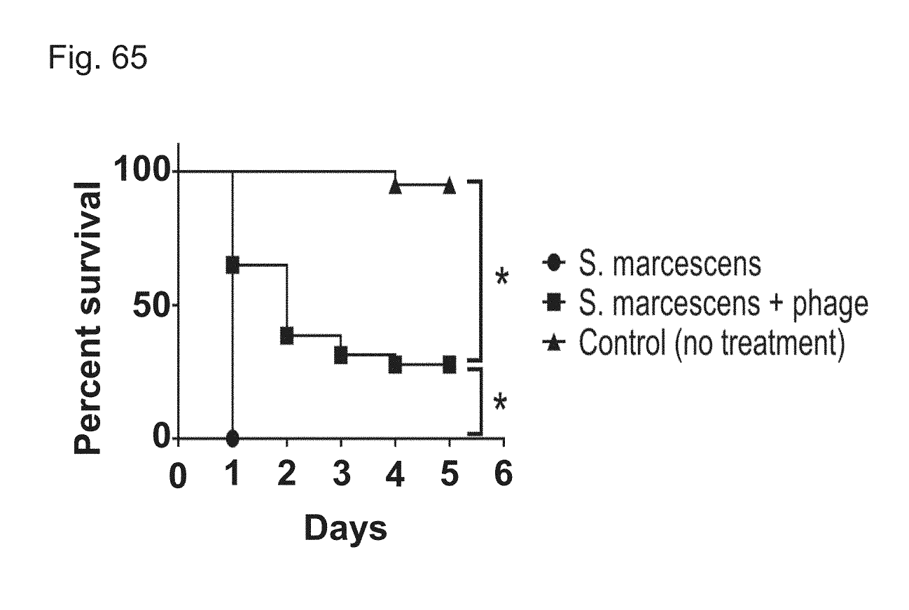

Flies in all conditions were placed in food bottles and incubated at 26° C., 16:8 light:dark cycle, at 60% humidity. The number of alive and dead flies were counted every day for four days after the pricking. All The flies pricked with S. marcescens alone were all dead within 24 hours of the treatment. In comparison, more than 60% of the flies in the phage treatment group, and all the flies in the untreated control group were alive at that time point (FIG. 65). Further, most of the flies in the phage treatment group and the negative control group went on to survive for four more days when the experiment was terminated.

To ascertain the reason of death of the flies, dead flies from both the S. marcescens and S. marcescens+phage pricked flies were homogenized and plated out. Four dead flies from each of the four replicates of both the S. marcescens and the S. marcescens+phage treatment were homogenized in 100 ul of TSB. A 1:100 dilution was also produced by diluting the homogenate in TSB. 10 ul of the concentrated homogenate as well as the 1:100 dilution was plated out onto TSA plates, and incubated overnight at 30° C. Upon inspection of the plates for bacteria growth, all the plates from the dead S. marcescens pricked flies had a lawn of bacteria growing on them, whereas the plates from the dead S. marcescens+phage pricked flies had no bacteria on them. This shows that in the absence of the phage, S. marcescens likely induced septic shock in the flies leading to their fatality. However, in the presence of the phage, the mortality may have been due to injury caused by the pricking with the needle.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for decreasing the fitness of an agricultural insect pest, the method comprising:
(a) providing a composition comprising (i) validamycin and (ii) an antimicrobial agent that targets one or more symbiotic microorganisms in the agricultural insect pest; and
(b) delivering said composition to the agricultural insect pest, whereby the fitness of the agricultural insect pest treated with the composition is decreased relative to an untreated agricultural insect pest.

2. The method of claim 1, wherein the antimicrobial agent is a chemical compound.

3. The method of claim 1, wherein the antimicrobial agent is an antibiotic, a polypeptide, a bacteriocin, a lysin, an antimicrobial peptide, a secondary metabolite, a small molecule, or a phage.

4. The method of claim 1, wherein the decrease in fitness of the agricultural insect pest is measured as a decrease in a physiological parameter of the insect.

5. The method of claim 1, wherein the decrease in fitness of the agricultural insect pest is measured as death of the insect.

6. The method of claim 1, wherein the delivery comprises delivering the composition to at least one habitat where the agricultural insect pest grows, lives, reproduces, feeds, or infests.

7. The method of claim 1, wherein the composition is formulated with an agriculturally acceptable carrier as a liquid, a solid, an aerosol, a paste, a gel, or a gas composition.

8. The method of claim 1, wherein the composition is delivered as a spray to an agricultural crop.

9. The method of claim 1, wherein the agricultural insect pest is an aphid, a member of the Pentatomidae, or a whitefly.

10. The method of claim 9, wherein the agricultural insect pest is an aphid.

11. The method of claim 10, wherein the aphid is Acyrthosiphon pisum.

12. The method of claim 10 or 11, wherein the symbiotic microorganism is Buchnera.

13. The method of claim 9, wherein the agricultural insect pest is a member of the Pentatomidae.

14. The method of claim 13, wherein the member of the Pentatomidae is a Nezara species or an Oebalus species.

15. The method of claim 9, wherein the agricultural insect pest is a whitefly.

16. The method of claim 15, wherein the whitefly is Bemisia tabaci.

17. The method of claim 15 or 16, wherein the symbiotic microorganism is Portiera aleyrodidarum.


SEQUENCE LISTING (241 sequences)

SEQ ID NO: 1 - DNA, 1526 bp, Carsonella ruddii
tatccagccacaggttcccctacagctaccttgttacgacttcaccccagttacaaatcataccgttgtaatagtaaaattacttatgatacaatttacttccatggtgtgacgggcggtgtgtacaaggctcgagaacgtattcaccgtaacattctgatttacgattactagcgattccaacttcatgaaatcgagttacagatttcaatccgaactaagaatattttttaagattagcattatgttgccatatagcatataactttttgtaatactcattgtagcacgtgtgtagccctacttataagggccatgatgacttgacgtcgtcctcaccttcctccaatttatcattggcagtttcttattagttctaatatatttttagtaaaataagataagggttgcgctcgttataggacttaacccaacatttcacaacacgagctgacgacagccatgcagcacctgtctcaaagctaaaaaagctttattatttctaataaattctttggatgtcaaaagtaggtaagatttttcgtgttgtatcgaattaaaccacatgctccaccgcttgtgcgagcccccgtcaattcatttgagttttaaccttgcggtcgtaatccccaggcggtcaacttaacgcgttagctttttcactaaaaatatataactttttttcataaaacaaaattacaattataatatttaataaatagttgacatcgtttactgcatggactaccagggtatctaatcctgtttgctccccatgctttcgtgtattagtgtcagtattaaaatagaaatacgccttcgccactagtattctttcagatatctaagcatttcactgctactcctgaaattctaatttcttcttttatactcaagtttataagtattaatttcaatattaaattactttaataaatttaaaaattaatttttaaaaacaacctgcacaccctttacgcccaataattccgattaacgcttgcacccctcgtattaccgcggctgctggcacgaagttagccggtgcttcttttacaaataacgtcaaagataatatttttttattataaaatctcttcttactttgttgaaagtgttttacaaccctaaggccttcttcacacacgcgatatagctggatcaagctttcgctcattgtccaatatcccccactgctgccttccgtaaaagtttgggccgtgtctcagtcccaatgtggttgttcatcctctaagatcaactacgaatcatagtcttgttaagcttttactttaacaactaactaattcgatataagctcttctattagcgaacgacattctcgttctttatccattaggatacatattgaattactatacatttctatatacttttctaatactaataggtagattcttatatattactcacccgttcgctgctaattatttttttaataattcgcacaacttgcatgtgttaagcttatcgctagcgttcaatctgagctatgatcaaactca

SEQ ID NO: 2 - DNA, 1536 bp, aleyrodidarum BT-B
aagagtttgatcatggctcagattgaacgctagcggcagacataacacatgcaagtcgagcggcatcatacaggttggcaagcggcgcacgggtgagtaatacatgtaaatatacctaaaagtggggaataacgtacggaaacgtacgctaataccgcataattattacgagataaagcaggggcttgataaaaaaaatcaaccttgcgcttttagaaaattacatgccggattagctagttggtagagtaaaagcctaccaaggtaacgatccgtagctggtctgagaggatgatcagccacactgggactgagaaaaggcccagactcctacgggaggcagcagtggggaatattggacaatggggggaaccctgatccagtcatgccgcgtgtgtgaagaaggcctttgggttgtaaagcactttcagcgaagaagaaaagttagaaaataaaaagttataactatgacggtactcgcagaagaagcaccggctaactccgtgccagcagccgcggtaagacggagggtgcaagcgttaatcagaattactgggcgtaaagggcatgtaggtggtttgttaagctttatgtgaaagccctatgcttaacataggaacggaataaagaactgacaaactagagtgcagaagaggaaggtagaattcccggtgtagcggtgaaatgcgtagatatctggaggaataccagttgcgaaggcgaccttctgggctgacactgacactgagatgcgaaagcgtggggagcaaacaggattagataccctggtagtccacgctgtaaacgatatcaactagccgttggattcttaaagaattttgtggcgtagctaacgcgataagttgatcgcctggggagtacggtcgcaaggctaaaactcaaatgaattgacgggggcccgcacaagcggtggagcatgtggtttaattcgatgcaacgcgcaaaaccttacctactcttgacatccaaagtactttccagagatggaagggtgccttagggaactttgagacaggtgctgcatggctgtcgtcagctcgtgttgtgaaatgttgggttaagtcccgtaacgagcgcaacccttgtccttagttgccaacgcataaggcgggaactttaaggagactgctggtgataaaccggaggaaggtggggacgacgtcaagtcatcatggcccttaagagtagggcaacacacgtgctacaatggcaaaaacaaagggtcgcaaaatggtaacatgaagctaatcccaaaaaaattgtcttagttcggattggagtctgaaactcgactccataaagtcggaatcgctagtaatcgtgaatcagaatgtcacggtgaatacgttctcgggccttgtacacaccgcccgtcacaccatggaagtgaaatgcaccagaagtggcaagtttaaccaaaaaacaggagaacagtcactacggtgtggttcatgactggggtgaagtcgtaacaaggtagctgtaggggaacctgtggctggatcacctccttaa

SEQ ID NO: 3 - DNA, 1540 bp, Buchnera aphidicola str. APS (Acyrthosiphon pisum)
agagtttgatcatggctcagattgaacgctggcggcaagcctaacacatgcaagtcgagcggcagcgagaagagagcttgctctctttgtcggcaagcggcaaacgggtgagtaatatctggggatctacccaaaagagggggataactactagaaatggtagctaataccgcataatgttgaaaaaccaaagtgggggaccttttggcctcatgcttttggatgaacccagacgagattagcttgttggtagagtaatagcctaccaaggcaacgatctctagctggtctgagaggataaccagccacactggaactgagacacggtccagactcctacgggaggcagcagtggggaatattgcacaatgggcgaaagcctgatgcagctatgccgcgtgtatgaagaaggccttagggttgtaaagtactttcagcggggaggaaaaaaataaaactaataattttatttcgtgacgttacccgcagaagaagcaccggctaactccgtgccagcagccgcggtaatacggagggtgcaagcgttaatcagaattactgggcgtaaagagcgcgtaggtggttttttaagtcaggtgtgaaatccctaggctcaacctaggaactgcatttgaaactggaaaactagagtttcgtagagggaggtagaattctaggtgtagcggtgaaatgcgtagatatctggaggaatacccgtggcgaaagcggcctcctaaacgaaaactgacactgaggcgcgaaagcgtggggagcaaacaggattagataccctggtagtccatgccgtaaacgatgtcgacttggaggttgtttccaagagaagtgacttccgaagctaacgcattaagtcgaccgcctggggagtacggccgcaaggctaaaactcaaatgaattgacgggggcccgcacaagcggtggagcatgtggtttaattcgatgcaacgcgaaaaaccttacctggtcttgacatccacagaattctttagaaataaagaagtgccttcgggagctgtgagacaggtgctgcatggctgtcgtcagctcgtgttgtgaaatgttgggttaagtcccgcaacgagcgcaacccttatcccctgttgccagcggttcggccgggaactcagaggagactgccggttataaaccggaggaaggtggggacgacgtcaagtcatcatggcccttacgaccagggctacacacgtgctacaatggtttatacaaagagaagcaaatctgcaaagacaagcaaacctcataaagtaaatcgtagtccggactggagtctgcaactcgactccacgaagtcggaatcgctagtaatcgtggatcagaatgccacggtgaatacgttcccgggccttgtacacaccgcccgtcacaccatgggagtgggttgcaaaagaagcaggtatcctaaccctttaaaaggaaggcgcttaccactttgtgattcatgactggggtgaagtcgtaacaaggtaaccgtaggggaacctgcggttggatcacctcctt

SEQ ID NO: 4 - DNA, 1552 bp, Buchnera aphidicola str. Sg (Schizaphis graminum)
aaactgaagagtttgatcatggctcagattgaacgctggcggcaagcctaacacatgcaagtcgagcggcagcgaaaagaaagcttgctttcttgtcggcgagcggcaaacgggtgagtaatatctggggatctgcccaaaagagggggataactactagaaatggtagctaataccgcataaagttgaaaaaccaaagtgggggaccttttttaaaggcctcatgcttttggatgaacccagacgagattagcttgttggtaaggtaaaagcttaccaaggcaacgatctctagctggtctgagaggataaccagccacactggaactgagacacggtccagactcctacgggaggcagcagtggggaatattgcacaatgggcgaaagcctgatgcagctatgccgcgtgtatgaagaaggccttagggttgtaaagtactttcagcggggaggaaaaaattaaaactaataattttattttgtgacgttacccgcagaagaagcaccggctaactccgtgccagcagccgcggtaatacggagggtgcgagcgttaatcagaattactgggcgtaaagagcacgtaggtggttttttaagtcagatgtgaaatccctaggcttaacctaggaactgcatttgaaactgaaatgctagagtatcgtagagggaggtagaattctaggtgtagcggtgaaatgcgtagatatctggaggaatacccgtggcgaaagcggcctcctaaacgaatactgacactgaggtgcgaaagcgtggggagcaaacaggattagataccctggtagtccatgccgtaaacgatgtcgacttggaggttgtttccaagagaagtgacttccgaagctaacgcgttaagtcgaccgcctggggagtacggccgcaaggctaaaactcaaatgaattgacgggggcccgcacaagcggtggagcatgtggtttaattcgatgcaacgcgaaaaaccttacctggtcttgacatccacagaattttttagaaataaaaaagtgccttcgggaactgtgagacaggtgctgcatggctgtcgtcagctcgtgttgtgaaatgttgggttaagtcccgcaacgagcgcaacccttatcccctgttgccagcggttcggccgggaactcagaggagactgccggttataaaccggaggaaggtggggacgacgtcaagtcatcatggcccttacgaccagggctacacacgtgctacaatggtttatacaaagagaagcaaatctgtaaagacaagcaaacctcataaagtaaatcgtagtccggactggagtctgcaactcgactccacgaagtcggaatcgctagtaatcgtggatcagaatgccacggtgaatacgttcccgggccttgtacacaccgcccgtcacaccatgggagtgggttgcaaaagaagcagatttcctaaccacgaaagtggaaggcgtctaccactttgtgattcatgactggggtgaagtcgtaacaaggtaaccgtaggggaacctgcggttggatcacctcctta

SEQ ID NO: 5 - DNA, 1566 bp, Buchnera aphidicola str. Bp (Baizongia pistaciae)
acttaaaattgaagagtttgatcatggctcagattgaacgctggcggcaagcttaacacatgcaagtcgagcggcatcgaagaaaagtttacttttctggcggcgagcggcaaacgggtgagtaacatctggggatctacctaaaagagggggacaaccattggaaacgatggctaataccgcataatgtttttaaataaaccaaagtaggggactaaaatttttagccttatgcttttagatgaacccagacgagattagcttgatggtaaggtaatggcttaccaaggcgacgatctctagctggtctgagaggataaccagccacactggaactgagatacggtccagactcctacgggaggcagcagtggggaatattgcacaatgggctaaagcctgatgcagctatgccgcgtgtatgaagaaggccttagggttgtaaagtactttcagcggggaggaaagaattatgtctaatatacatattttgtgacgttacccgaagaagaagcaccggctaactccgtgccagcagccgcggtaatacggagggtgcgagcgttaatcagaattactgggcgtaaagagcacgtaggcggtttattaagtcagatgtgaaatccctaggcttaacttaggaactgcatttgaaactaatagactagagtctcatagagggaggtagaattctaggtgtagcggtgaaatgcgtagatatctagaggaatacccgtggcgaaagcgacctcctaaatgaaaactgacgctgaggtgcgaaagcgtggggagcaaacaggattagataccctggtagtccatgctgtaaacgatgtcgacttggaggttgtttcctagagaagtggcttccgaagctaacgcattaagtcgaccgcctggggagtacggtcgcaaggctaaaactcaaatgaattgacgggggcccgcacaagcggtggagcatgtggtttaattcgatgcaacgcgaagaaccttacctggtcttgacatccatagaattttttagagataaaagagtgccttagggaactatgagacaggtgctgcatggctgtcgtcagctcgtgttgtgaaatgttgggttaagtcccgcaacgagcgcaacccctatcctttgttgccatcaggttatgctgggaactcagaggagactgccggttataaaccggaggaaggtggggatgacgtcaagtcatcatggcccttacgaccagggctacacacgtgctacaatggcatatacaaagagatgcaactctgcgaagataagcaaacctcataaagtatgtcgtagtccggactggagtctgcaactcgactccacgaagtaggaatcgctagtaatcgtggatcagaatgccacggtgaatacgttcccgggccttgtacacaccgcccgtcacaccatgggagtgggttgcaaaagaagcaggtagcttaaccagattattttattggagggcgcttaccactttgtgattcatgactggggtgaagtcgtaacaaggtaaccgtaggggaacctgcggttggatcacctcctta

SEQ ID NO: 6 - DNA, 828 bp, Buchnera aphidicola BCc
atgagatcattaatatataaaaatcatgttccaattaaaaaattaggacaaaattttttacagaataaagaaattattaatcagataattaatttaataaatattaataaaaatgataatattattgaaataggatcaggattaggagcgttaacttttcctatttgtagaatcattaaaaaaatgatagtattagaaattgatgaagatcttgtgttttttttaactcaaagtttatttattaaaaaattacaaattataattgctgatattataaaatttgatttttgttgttttttttctttacagaaatataaaaaatataggtttattggtaatttaccatataatattgctactatattttttttaaaaacaattaaatttctttataatataattgatatgcattttatgtttcaaaaagaagtagcaaagagattattagctactcctggtactaaagaatatggtagattaagtattattgcacaatatttttataagatagaaactgttattaatgttaataaatttaatttttttcctactcctaaagtagattctacttttttacgatttactcctaaatattttaatagtaaatataaaatagataaacatttttctgttttagaattaattactagattttcttttcaacatagaagaaaatttttaaataataatttaatatctttattttctacaaaagaattaatttctttagatattgatccatattcaagagcagaaaatgtttctttaattcaatattgtaaattaatgaaatattatttgaaaagaaaaattttatgtttagattaa

SEQ ID NO: 7 - DNA, 921 bp, Buchnera aphidicola (Cinara tujafilina)
ttatcttatttcacatatacgtaatattgcgctgcgtgcacgaggatttttttgaatttcagatatatttggtttaatacgtttaataaaacgtatttttttttttatttttcttatttgcaattcagtaataggaagttttttaggtatatttggataattactgtaattcttaataaagttttttacaatcctatcttcaatagaatgaaaactaataatagcaatttttgatccggaatgtaatatgttaataataatttttaatattttatgtaattcatttatttcttggttaatatatattcgaaaagcttgaaatgttctcgtagctggatgtttaaatttgtcatattttgggattgatttttttatgatttgaactaactctaacgtgcttgttatggtttttttttttatttgtaatatgatggctcgggatattttttttgcgtatttttcttcgccaaaattttttattacctgttctattgttttttggtttgttttttttaaccattgactaactgatattccagatttagggttcatacgcatatctaaaggtccatcattcataaatgaaaatcctcggatactagaatttaactgtattgaagaaatacctaaatctaataatattccatctattttatctctatttttttctttttttaatattttttcaatattagaaaatttacctaaaaatattttaaatcgcgaatcttttattttttttccgatttttatagattgtgggtcttgatcaatactatataactttccattaacccctaattcttgaagaattgcttttgaatgaccaccacctccaaatgtacaatcaacatatgtaccgtctttttttatttttaagtattgtatgatttcttttgttaaaacaggtttatgaatcat

SEQ ID NO: 8 - DNA, 822 bp, Buchnera aphidicola str. G002 (Myzus persicae)
atgaaaagtataaaaacttttaaaaaacactttcctgtgaaaaaatatggacaaaattttcttattaataaagagatcataaaaaatattgttaaaaaaattaatccaaatatagaacaaacattagtagaaatcggaccaggattagctgcattaactgagcccatatctcagttattaaaagagttaatagttattgaaatagactgtaatctattatattttttaaaaaaacaaccattttattcaaaattaatagttttttgtcaagatgctttaaactttaattatacaaatttattttataaaaaaaataaattaattcgtatttttggtaatttaccatataatatctctacatctttaattatttttttatttcaacacattagagtaattcaagatatgaattttatgcttcaaaaagaagttgctgcaagattaattgcattacctggaaataaatattacggtcgtttgagcattatatctcaatattattgtgatatcaaaattttattaaatgttgctcctgaagatttttggcctattccgagagttcattctatatttgtaaatttaacacctcatcataattctccttattttgtttatgatattaatattttaagccttattacaaataaggctttccaaaatagaagaaaaatattacgtcatagtttaaaaaatttattttctgaaacaactttattaaatttagatattaatcccagattaagagctgaaaatatttctgtttttcagtattgtcaattagctaattatttgtataaaaaaaattatactaaaaaaaattaa

SEQ ID NO: 9 - DNA, 822 bp, Buchnera aphidicola str. Ak (Acyrthosiphon kondoi)
attataaaaaattttaaaaaacattttcctttaaaaaggtatggacaaaattttcttgtcaatacaaaaactattcaaaagataattaatataattaatccaaacaccaaacaaacattagtggaaattggacctggattagctgcattaacaaaaccaatttgtcaattattagaagaattaattgttattgaaatagatcctaatttattgtttttattaaaaaaacgttcattttattcaaaattaacagttttttatcaagacgctttaaatttcaattatacagatttgttttataagaaaaatcaattaattcgtgtttttggaaacttgccatataatatttctacatctttaattatttctttattcaatcatattaaagttattcaagatatgaattttatgttacagaaagaggttgctgaaagattaatttctattcctggaaataaatcttatggccgtttaagcattatttctcagtattattgtaaaattaaaatattattaaatgttgtacctgaagattttcgacctataccgaaagtgcattctgtttttatcaatttaactcctcataccaattctccatattttgtttatgatacaaatatcctcagttctatcacaagaaatgcttttcaaaatagaaggaaaattttgcgtcatagtttaaaaaatttattttctgaaaaagaactaattcaattagaaattaatccaaatttacgagctgaaaatatttctatctttcagtattgtcaattagctgattatttatataaaaaattaaataatcttgtaaaaatcaattaa

SEQ ID NO: 10 - DNA, 822 bp, Buchnera aphidicola str. Ua (Uroleucon ambrosiae)
atgatactaaataaatataaaaaatttattcctttaaaaagatacggacaaaattttcttgtaaatagagaaataatcaaaaatattatcaaaataattaatcctaaaaaaacgcaaacattattagaaattggaccgggtttaggtgcgttaacaaaacctatttgtgaatttttaaatgaacttatcgtcattgaaatagatcctaatatattatcttttttaaagaaatgtatattttttgataaattaaaaatatattgtcataatgctttagattttaattataaaaatatattctataaaaaaagtcaattaattcgtatttttggaaatttaccatataatatttctacatctttaataatatatttatttcggaatattgatattattcaagatatgaattttatgttacaacaagaagtggctaaaagattagttgctattcctggtgaaaaactttatggtcgtttaagtattatatctcaatattattgtaatattaaaatattattacatattcgacctgaaaattttcaacctattcctaaagttaattcaatgtttgtaaatttaactccgcatattcattctccttattttgtttatgatattaatttattaactagtattacaaaacatgcttttcaacatagaagaaaaatattgcgtcatagtttaagaaattttttttctgagcaagatttaattcatttagaaattaatccaaatttaagagctgaaaatgtttctattattcaatattgtcaattggctaataatttatataaaaaacataaacagtttattaataattaa

SEQ ID NO: 11 - DNA, 816 bp, Buchnera aphidicola (Aphis glycines)
atgaaaaagcatattcctataaaaaaatttagtcaaaattttcttgtagatttgagtgtgattaaaaaaataattaaatttattaatccgcagttaaatgaaatattggttgaaattggaccgggattagctgctatcactcgacctatttgtgatttgatagatcatttaattgtgattgaaattgataaaattttattagatagattaaaacagttctcattttattcaaaattaacagtatatcatcaagatgctttagcatttgattacataaagttatttaataaaaaaaataaattagttcgaatttttggtaatttaccatatcatgtttctacgtctttaatattgcatttatttaaaagaattaatattattaaagatatgaattttatgctacaaaaagaagttgctgaacgtttaattgcaactccaggtagtaaattatatggtcgtttaagtattatttctcaatattattgtaatataaaagttttattgcatgtgtcttcaaaatgttttaaaccagttcctaaagtagaatcaatttttcttaatttgacaccttatactgattatttcccttattttacttataatgtaaacgttcttagttatattacaaatttagcttttcaaaaaagaagaaaaatattacgtcatagtttaggtaaaatattttctgaaaaagtttttataaaattaaatattaatcccaaattaagacctgagaatatttctatattacaatattgtcagttatctaattatatgatagaaaataatattcatcaggaacatgtttgtatttaa

SEQ ID NO: 12 - DNA, 1463 bp, Annandia pinicola
agattgaacgctggcggcatgccttacacatgcaagtcgaacggtaacaggtcttcggacgctgacgagtggcgaacgggtgagtaatacatcggaacgtgcccagtcgtgggggataactactcgaaagagtagctaataccgcatacgatctgaggatgaaagcgggggaccttcgggcctcgcgcgattggagcggccgatggcagattaggtagttggtgggataaaagcttaccaagccgacgatctgtagctggtctgagaggacgaccagccacactggaactgagatacggtccagactcttacgggaggcagcagtggggaatattgcacaatgggcgcaagcctgatgcagctatgtcgcgtgtatgaagaagaccttagggttgtaaagtactttcgatagcataagaagataatgagactaataattttattgtctgacgttagctatagaagaagcaccggctaactccgtgccagcagccgcggtaatacggggggtgctagcgttaatcggaattactgggcgtaaagagcatgtaggtggtttattaagtcagatgtgaaatccctggacttaatctaggaactgcatttgaaactaataggctagagtttcgtagagggaggtagaattctaggtgtagcggtgaaatgcatagatatctagaggaatatcagtggcgaaggcgaccttctggacgataactgacgctaaaatgcgaaagcatgggtagcaaacaggattagataccctggtagtccatgctgtaaacgatgtcgactaagaggttggaggtataacttttaatctctgtagctaacgcgttaagtcgaccgcctggggagtacggtcgcaaggctaaaactcaaatgaattgacgggggcctgcacaagcggtggagcatgtggtttaattcgatgcaacgcgtaaaaccttacctggtcttgacatccacagaattttacagaaatgtagaagtgcaatttgaactgtgagacaggtgctgcatggctgtcgtcagctcgtgttgtgaaatgttgggttaagtcccgcaacgagcgcaacccttgtcctttgttaccataagatttaaggaactcaaaggagactgccggtgataaactggaggaaggcggggacgacgtcaagtcatcatggcccttatgaccagggctacacacgtgctacaatggcatatacaaagagatgcaatattgcgaaataaagccaatcttataaaatatgtcctagttcggactggagtctgcaactcgactccacgaagtcggaatcgctagtaatcgtggatcagcatgccacggtgaatatgtttccaggccttgtacacaccgcccgtcacaccatggaagtggattgcaaaagaagtaagaaaattaaccttcttaacaaggaaataacttaccactttgtgactcataactggggtga

SEQ ID NO: 13 - DNA, 1554 bp, Moranella endobia
tctttttggtaaggaggtgatccaaccgcaggttcccctacggttaccttgttacgacttcaccccagtcatgaatcacaaagtggtaagcgccctcctaaaaggttaggctacctacttcttttgcaacccacttccatggtgtgacgggcggtgtgtacaaggcccgggaacgtattcaccgtggcattctgatccacgattactagcgattcctacttcatggagtcgagttgcagactccaatccggactacgacgcactttatgaggtccgctaactctcgcgagcttgcttctctttgtatgcgccattgtagcacgtgtgtagccctactcgtaagggccatgatgacttgacgtcatccccaccttcctccggtttatcaccggcagtctcctttgagttcccgaccgaatcgctggcaaaaaaggataagggttgcgctcgttgcgggacttaacccaacatttcacaacacgagctgacgacagccatgcagcacctgtctcagagttcccgaaggtaccaaaacatctctgctaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgatttaacgcgttaactacgaaagccacagttcaagaccacagctttcaaatcgacatagtttacggcgtggactaccagggtatctaatcctgtttgctccccacgctttcgtacctgagcgtcagtattcgtccagggggccgccttcgccactggtattcctccagatatctacacatttcaccgctacacctggaattctacccccctctacgagactctagcctatcagtttcaaatgcagttcctaggttaagcccagggatttcacatctgacttaataaaccgcctacgtactctttacgcccagtaattccgattaacgcttgcaccctccgtattaccgcggctgctggcacggagttagccggtgcttcttctgtaggtaacgtcaatcaataaccgtattaaggatattgccttcctccctactgaaagtgctttacaacccgaaggccttcttcacacacgcggcatggctgcatcagggtttcccccattgtgcaatattccccactgctgcctcccgtaggagtctggaccgtgtctcagttccagtgtggctggtcatcctctcagaccagctagggatcgtcgcctaggtaagctattacctcacctactagctaatcccatctgggttcatctgaaggtgtgaggccaaaaggtcccccactttggtcttacgacattatgcggtattagctaccgtttccagcagttatccccctccatcaggcagatccccagactttactcacccgttcgctgctcgccggcaaaaaagtaaacttttttccgttgccgctcaacttgcatgtgttaggcctgccgccagcgttcaatctgagccatgatcaaactcttcaattaaa

SEQ ID NO: 14 - DNA, 1539 bp, Ishikawaella capsulata Mpkobe
aaattgaagagtttgatcatggctcagattgaacgctagcggcaagcttaacacatgcaagtcgaacggtaacagaaaaaagcttgcttttttgctgacgagtggcggacgggtgagtaatgtctggggatctacctaatggcgggggataactactggaaacggtagctaataccgcataatgttgtaaaaccaaagtgggggaccttatggcctcacaccattagatgaacctagatgggattagcttgtaggtggggtaaaggctcacctaggcaacgatccctagctggtctgagaggatgaccagccacactggaactgagatacggtccagactcctacgggaggcagcagtggggaatcttgcacaatgggcgcaagcctgatgcagctatgtcgcgtgtatgaagaaggccttagggttgtaaagtactttcatcggggaagaaggatatgagcctaatattctcatatattgacgttacctgcagaagaagcaccggctaactccgtgccagcagccgcggtaacacggagggtgcgagcgttaatcggaattactgggcgtaaagagcacgtaggtggtttattaagtcatatgtgaaatccctgggcttaacctaggaactgcatgtgaaactgataaactagagtttcgtagagggaggtggaattccaggtgtagcggtgaaatgcgtagatatctggaggaatatcagaggcgaaggcgaccttctggacgaaaactgacactcaggtgcgaaagcgtggggagcaaacaggattagataccctggtagtccacgctgtaaacaatgtcgactaaaaaactgtgagcttgacttgtggtttttgtagctaacgcattaagtcgaccgcctggggagtacggccgcaaggttaaaactcaaatgaattgacgggggtccgcacaagcggtggagcatgtggtttaattcgatgcaacgcgaaaaaccttacctggtcttgacatccagcgaattatatagaaatatataagtgcctttcggggaactctgagacgctgcatggctgtcgtcagctcgtgttgtgaaatgttgggttaagtcccgcaacgagcgcccttatcctctgttgccagcggcatggccgggaactcagaggagactgccagtattaaactggaggaaggtggggatgacgtcaagtcatcatggcccttatgaccagggctacacacgtgctacaatggtgtatacaaagagaagcaatctcgcaagagtaagcaaaactcaaaaagtacatcgtagttcggattagagtctgcaactcgactctatgaagtaggaatcgctagtaatcgtggatcagaatgccacggtgaatacgttctctggccttgtacacaccgcccgtcacaccatgggagtaagttgcaaaagaagtaggtagcttaacctttataggagggcgcttaccactttgtgatttatgactggggtgaagtcgtaacaaggtaactgtaggggaacctgtggttggattacctcctta

SEQ ID NO: 15 - DNA, 1561 bp, Baumannia cicadellinicola
ttcaattgaagagtttgatcatggctcagattgaacgctggcggtaagcttaacacatgcaagtcgagcggcatcggaaagtaaattaattactttgccggcaagcggcgaacgggtgagtaatatctggggatctaccttatggagagggataactattggaaacgatagctaacaccgcataatgtcgtcagaccaaaatgggggacctaatttaggcctcatgccataagatgaacccagatgagattagctagtaggtgagataatagctcacctaggcaacgatctctagttggtctgagaggatgaccagccacactggaactgagacacggtccagactcctacgggaggcagcagtggggaatcttgcacaatgggggaaaccctgatgcagctataccgcgtgtgtgaagaaggccttcgggttgtaaagcactttcagcggggaagaaaatgaagttactaataataattgtcaattgacgttacccgcaaaagaagcaccggctaactccgtgccagcagccgcggtaagacggagggtgcaagcgttaatcggaattactgggcgtaaagcgtatgtaggcggtttatttagtcaggtgtgaaagccctaggcttaacctaggaattgcatttgaaactggtaagctagagtctcgtagagggggggagaattccaggtgtagcggtgaaatgcgtagagatctggaagaataccagtggcgaaggcgcccccctggacgaaaactgacgctcaagtacgaaagcgtggggagcaaacaggattagataccctggtagtccacgctgtaaacgatgtcgatttgaaggttgtagccttgagctatagctttcgaagctaacgcattaaatcgaccgcctggggagtacgaccgcaaggttaaaactcaaatgaattgacgggggcccgcacaagcggtggagcatgtggtttaattcgatacaacgcgaaaaaccttacctactcttgacatccagagtataaagcagaaaagctttagtgccttcgggaactctgagacaggtgctgcatggctgtcgtcagctcgtgttgtgaaatgttgggttaagtcccgcaacgagcgcaacccttatcctttgttgccaacgattaagtcgggaactcaaaggagactgccggtgataaaccggaggaaggtgaggataacgtcaagtcatcatggcccttacgagtagggctacacacgtgctacaatggtgcatacaaagagaagcaatctcgtaagagttagcaaacctcataaagtgcatcgtagtccggattagagtctgcaactcgactctatgaagtcggaatcgctagtaatcgtggatcagaatgccacggtgaatacgttcccgggccttgtacacaccgcccgtcacaccatgggagtgtattgcaaaagaagttagtagcttaactcataatacgagagggcgcttaccactttgtgattcataactggggtgaagtcgtaacaaggtaaccgtaggggaacctgcggttggatcacctccttacactaaa

SEQ ID NO: 16 - DNA, 1464 bp, Sodalis like
attgaacgctggcggcaggcctaacacatgcaagtcgagcggcagcgggaagaagcttgcttctttgccggcgagcggcggacgggtgagtaatgtctggggatctgcccgatggagggggataactactggaaacggtagctaataccgcataacgtcgcaagaccaaagtgggggaccttcgggcctcacaccatcggatgaacccaggtgggattagctagtaggtggggtaatggctcacctaggcgacgatccctagctggtctgagaggatgaccagtcacactggaactgagacacggtccagactcctacgggaggcagcagtggggaatattgcacaatgggggaaaccctgatgcagccatgccgcgtgtgtgaagaaggccttcgggttgtaaagcactttcagcggggaggaaggcgatggcgttaatagcgctatcgattgacgttacccgcagaagaagcaccggctaactccgtgccagcagccgcggtaatacggagggtgcgagcgttaatcggaattactgggcgtaaagcgtacgcaggcggtctgttaagtcagatgtgaaatccccgggctcaacctgggaactgcatttgaaactggcaggctagagtctcgtagaggggggtagaattccaggtgtagcggtgaaatgcgtagagatctggaggaataccggtggcgaaggcggccccctggacgaagactgacgctcaggtacgaaagcgtggggagcaaacaggattagataccctggtagtccacgctgtaaacgatgtcgatttgaaggttgtggccttgagccgtggctttcggagctaacgtgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaacttggcagagatgctttggtgccttcgggaactctgagacaggtgctgcatggctgtcgtcagctcgtgttgtgaaatgttgggttaagtcccgcaacgagcgcaacccttatcctttattgccagcgattcggtcgggaactcaaaggagactgccggtgataaaccggaggaaggtggggatgacgtcaagtcatcatggcccttacgagtagggctacacacgtgctacaatggcgcatacaaagagaagcgatctcgcgagagtcagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtg

SEQ ID NO: 17 - DNA, 1465 bp, Hartigia pinicola
agatttaacgctggcggcaggcctaacacatgcaagtcgagcggtaccagaagaagcttgcttcttgctgacgagcggcggacgggtgagtaatgtatggggatctgcccgacagagggggataactattggaaacggtagctaataccgcataatctctgaggagcaaagcaggggaacttcggtccttgcgctatcggatgaacccatatgggattagctagtaggtgaggtaatggctcccctaggcaacgatccctagctggtctgagaggatgatcagccacactgggactgagacacggcccagactcctacgggaggcagcagtggggaatattgcacaatgggcgaaagcctgatgcagccatgccgcgtgtatgaagaaggctttagggttgtaaagtactttcagtcgagaggaaaacattgatgctaatatcatcaattattgacgtttccgacagaagaagcaccggctaactccgtgccagcagccgcggtaatacggagggtgcaagcgttaatcggaattactgggcgtaaagcgcacgcaggcggttaattaagttagatgtgaaagccccgggcttaacccaggaatagcatataaaactggtcaactagagtattgtagaggggggtagaattccatgtgtagcggtgaaatgcgtagagatgtggaggaataccagtggcgaaggcggccccctggacaaaaactgacgctcaaatgcgaaagcgtggggagcaaacaggattagataccctggtagtccatgctgtaaacgatgtcgatttggaggttgttcccttgaggagtagcttccgtagctaacgcgttaaatcgaccgcctgggggagtacgactgcaaggttaaaactcaaatgaattgacgggggcccgcacaagcggtggagcatgtggtttaattcgatgcaacgcgaaaaaccttacctactcttgacatccagataatttagcagaaatgctttagtaccttcgggaaatctgagacaggtgctgcatggctgtcgtcagctcgtgttgtgaaatgttgggttaagtcccgcaacgagcgcaacccttatcctttgttgccagcgattaggtcgggaactcaaaggagactgccggtgataaaccggaggaaggtggggatgacgtcaagtcatcatggcccttacgagtagggctacacacgtgctacaatggcatatacaaagggaagcaacctcgcgagagcaagcgaaactcataaattatgtcgtagttcagattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtaacttaaccttatggaaagcgcttaccactttgtgattcataactggggtg

SEQ ID NO: 18 - DNA, 1571 bp, Tremblaya phenacola
aggtaatccagccacaccttccagtacggctaccttgttacgacttcaccccagtcacaacccttaccttcggaactgccctcctcacaactcaaaccaccaaacacttttaaatcaggttgagagaggttaggcctgttacttctggcaagaattatttccatggtgtgacgggcggtgtgtacaagacccgagaacatattcaccgtggcatgctgatccacgattactagcaattccaacttcatgcactcgagtttcagagtacaatccgaactgaggccggctttgtgagattagctcccttttgcaagttggcaactctttggtccggccattgtatgatgtgtgaagccccacccataaaggccatgaggacttgacgtcatccccaccttcctccaacttatcgctggcagtctctttaaggtaactgactaatccagtagcaattaaagacaggggttgcgctcgttacaggacttaacccaacatctcacgacacgagctgacgacagccatgcagcacctgtgcactaattctctttcaagcactcccgcttctcaacaggatcttagccatatcaaaggtaggtaaggtttttcgcgttgcatcgaattaatccacatcatccactgcttgtgcgggtccccgtcaattcctttgagttttaaccttgcggccgtactccccaggcggtcgacttgtgcgttagctgcaccactgaaaaggaaaactgcccaatggttagtcaacatcgtttagggcatggactaccagggtatctaatcctgtttgctccccatgctttagtgtctgagcgtcagtaacgaaccaggaggctgcctacgctttcggtattcctccacatctctacacatttcactgctacatgcggaattctacctccccctctcgtactccagcctgccagtaactgccgcattctgaggttaagcctcagcctttcacagcaatcttaacaggcagcctgcacaccctttacgcccaataaatctgattaacgctcgcaccctacgtattaccgcggctgctggcacgtagtttgccggtgcttattctttcggtacagtcacaccaccaaattgttagttgggtggctttctttccgaacaaaagtgctttacaacccaaaggccttcttcacacacgcggcattgctggatcaggcttccgcccattgtccaagattcctcactgctgccttcctcagaagtctgggccgtgtctcagtcccagtgtggctggccgtcctctcagaccagctaccgatcattgccttgggaagccattacctttccaacaagctaatcagacatcagccaatctcagagcgcaaggcaattggtcccctgctttcattctgcttggtagagaactttatgcggtattaattaggctttcacctagctgtcccccactctgaggcatgttctgatgcattactcacccgtttgccacttgccaccaagcctaagcccgtgttgccgttcgacttgcatgtgtaaggcatgccgctagcgttcaatctgagccaggatcaaactct

SEQ ID NO: 19 - DNA, 1535 bp, Tremblaya princeps
agagtttgatcctggctcagattgaacgctagcggcatgcattacacatgcaagtcgtacggcagcacgggcttaggcctggtggcgagtggcgaacgggtgagtaacgcctcggaacgtgccttgtagtgggggatagcctggcgaaagccagattaataccgcatgaagccgcacagcatgcgcggtgaaagtgggggattctagcctcacgctactggatcggccggggtctgattagctagttggcggggtaatggcccaccaaggcttagatcagtagctggtctgagaggacgatcagccacactgggactgagacacggcccagactcctacgggaggcagcagtggggaatcttggacaatgggcgcaagcctgatccagcaatgccgcgtgtgtgaagaaggccttcgggtcgtaaagcacttttgttcgggatgaaggggggcgtgcaaacaccatgccctcttgacgataccgaaagaataagcaccggctaactacgtgccagcagccgcggtaatacgtagggtgcgagcgttaatcggaatcactgggcgtaaagggtgcgcgggtggtttgccaagacccctgtaaaatcctacggcccaaccgtagtgctgcggaggttactggtaagcttgagtatggcagaggggggtagaattccaggtgtagcggtgaaatgcgtagatatctggaggaataccgaaggcgaaggcaaccccctgggccatcactgacactgaggcacgaaagcgtggggagcaaacaggattagataccctggtagtccacgccctaaaccatgtcgactagttgtcggggggagccctttttcctcggtgacgaagctaacgcatgaagtcgaccgcctggggagtacgaccgcaaggttaaaactcaaaggaattgacggggacccgcacaagcggtggatgatgtggattaattcgatgcaacgcgaaaaaccttacctacccttgacatggcggagattctgccgagaggcggaagtgctcgaaagagaatccgtgcacaggtgctgcatggctgtcgtcagctcgtgtcgtgagatgttgggttaagtcccataacgagcgcaacccccgtctttagttgctaccactggggcactctatagagactgccggtgataaaccggaggaaggtggggacgacgtcaagtcatcatggcctttatgggtagggcttcacacgtcatacaatggctggagcaaagggtcgccaactcgagagagggagctaatcccacaaacccagccccagttcggattgcactctgcaactcgagtgcatgaagtcggaatcgctagtaatcgtggatcagcatgccacggtgaatacgttctcgggtcttgtacacaccgcccgtcacaccatgggagtaagccgcatcagaagcagcctccctaaccctatgctgggaaggaggctgcgaaggtggggtctatgactggggtgaagtcgtaacaaggtagccgtaccggaaggtgcggctggattacct

SEQ ID NO: 20 - DNA, 1450 bp, Nasuia deltocephalinicola
agtttaatcctggctcagatttaacgcttgcgacatgcctaacacatgcaagttgaacgttgaaaatatttcaaagtagcgtataggtgagtataacatttaaacataccttaaagttcggaataccccgatgaaaatcggtataataccgtataaaagtatttaagaattaaagcggggaaaacctcgtgctataagattgttaaatgcctgattagtttgttggtttttaaggtaaaagcttaccaagactttgatcagtagctattctgtgaggatgtatagccacattgggattgaaataatgcccaaacctctacggagggcagcagtggggaatattggacaatgagcgaaagcttgatccagcaatgtcgcgtgtgcgattaagggaaactgtaaagcacttttttttaagaataagaaattttaattaataattaaaatttttgaatgtattaaaagaataagtaccgactaatcacgtgccagcagtcgcggtaatacgtggggtgcgagcgttaatcggatttattgggcgtaaagtgtattcaggctgcttaaaaagatttatattaaatatttaaattaaatttaaaaaatgtataaattactattaagctagagtttagtataagaaaaaagaattttatgtgtagcagtgaaatgcgttgatatataaaggaacgccgaaagcgaaagcatttttctgtaatagaactgacgcttatatacgaaagcgtgggtagcaaacaggattagataccctggtagtccacgccctaaactatgtcaattaactattagaattttttttagtggtgtagctaacgcgttaaattgaccgcctgggtattacgatcgcaagattaaaactcaaaggaattgacggggaccagcacaagcggtggatgatgtggattaattcgatgatacgcgaaaaaccttacctgcccttgacatggttagaattttattgaaaaataaaagtgcttggaaaagagctaacacacaggtgctgcatggctgtcgtcagctcgtgtcgtgagatgttgggttaagtcccgcaacgagcgcaacccctactcttagttgctaattaaagaactttaagagaacagctaacaataagtttagaggaaggaggggatgacttcaagtcctcatggcccttatgggcagggcttcacacgtcatacaatggttaatacaaaaagttgcaatatcgtaagattgagctaatctttaaaattaatcttagttcggattgtactctgcaactcgagtacatgaagttggaatcgctagtaatcgcggatcagcatgccgcggtgaatagtttaactggtcttgtacacaccgcccgtcacaccatggaaataaatcttgttttaaatgaagtaatatattttatcaaaacaggttttgtaaccggggtgaagtcgtaaca

SEQ ID NO: 21 - DNA, 1536 bp, Zinderia insecticola CARI
atataaataagagtttgatcctggctcagattgaacgctagcggtatgctttacacatgcaagtcgaacgacaatattaaagcttgctttaatataaagtggcgaacgggtgagtaatatatcaaaacgtaccttaaagtgggggataactaattgaaaaattagataataccgcatattaatcttaggatgaaaataggaataatatcttatgcttttagatcggttgatatctgattagctagttggtagggtaaatgcttaccaaggcaatgatcagtagctggttttagcgaatgatcagccacactggaactgagacacggtccagacttctacggaaggcagcagtggggaatattggacaatgggagaaatcctgatccagcaataccgcgtgagtgatgaaggccttagggtcgtaaaactcttttgttaggaaagaaataattttaaataatatttaaaattgatgacggtacctaaagaataagcaccggctaactacgtgccagcagccgcggtaatacgtagggtgcaagcgttaatcggaattattgggcgtaaagagtgcgtaggctgttatataagatagatgtgaaatacttaagcttaacttaagaactgcatttattactgtttaactagagtttattagagagaagtggaattttatgtgtagcagtgaaatgcgtagatatataaaggaatatcgatggcgaaggcagcttcttggaataatactgacgctgaggcacgaaagcgtggggagcaaacaggattagataccctggtagtccacgccctaaactatgtctactagttattaaattaaaaataaaatttagtaacgtagctaacgcattaagtagaccgcctggggagtacgatcgcaagattaaaactcaaaggaattgacggggacccgcacaagcggtggatgatgtggattaattcgatgcaacacgaaaaaccttacctactcttgacatgtttggaattttaaagaaatttaaaagtgcttgaaaaagaaccaaaacacaggtgctgcatggctgtcgtcagctcgtgtcgtgagatgttgggttaagtcccgcaacgagcgcaacccttgttattatttgctaataaaaagaactttaataagactgccaatgacaaattggaggaaggtggggatgacgtcaagtcctcatggcccttatgagtagggcttcacacgtcatacaatgatatatacaatgggtagcaaatttgtgaaaatgagccaatccttaaagtatatcttagttcggattgtagtctgcaactcgactacatgaagttggaatcgctagtaatcgcggatcagcatgccgcggtgaatacgttctcgggtcttgtacacaccgcccgtcacaccatggaagtgatttttaccagaaattatttgtttaacctttattggaaaaaaataattaaggtagaattcatgactggggtgaagtcgtaacaaggtagcagtatcggaaggtgcggctggattacattttaaat

SEQ ID NO: 22 - DNA, 1423 bp, Hodgkinia
aatgctggcggcaggcctaacacatgcaagtcgagcggacaacgttcaaacgttgttagcggcgaacgggtgagtaatacgtgagaatctacccatcccaacgtgataacatagtcaacaccatgtcaataacgtatgattcctgcaacaggtaaagattttatcggggatggatgagctcacgctagattagctagttggtgagataaaagcccaccaaggccaagatctatagctggtctggaaggatggacagccacattgggactgagacaaggcccaaccctctaaggagggcagcagtgaggaatattggacaatgggcgtaagcctgatccagccatgccgcatgagtgattgaaggtccaacggactgtaaaactcttttctccagagatcataaatgatagtatctggtgatataagctccggccaacttcgtgccagcagccgcggtaatacgaggggagcgagtattgttcggttttattgggcgtaaagggtgtccaggttgctaagtaagttaacaacaaaatcttgagattcaacctcataacgttcggttaatactactaagctcgagcttggatagagacaaacggaattccgagtgtagaggtgaaattcgttgatacttggaggaacaccagaggcgaaggcggtttgtcataccaagctgacactgaagacacgaaagcatggggagcaaacaggattagataccctggtagtccatgccctaaacgttgagtgctaacagttcgatcaagccacatgctatgatccaggattgtacagctaacgcgttaagcactccgcctgggtattacgaccgcaaggttaaaactcaaaggaattgacggagacccgcacaagcggtggagcatgtggtttaattcgaagctacacgaagaaccttaccagcccttgacataccatggccaaccatcctggaaacaggatgttgttcaagttaaacccttgaaatgccaggaacaggtgctgcatggctgttgtcagttcgtgtcgtgagatgtatggttaagtcccaaaacgaacacaaccctcacccatagttgccataaacacaattgggttctctatgggtactgctaacgtaagttagaggaaggtgaggaccacaacaagtcatcatggcccttatgggctgggccacacacatgctacaatggtggttacaaagagccgcaacgttgtgagaccgagcaaatctccaaagaccatctcagtccggattgtactctgcaacccgagtacatgaagtaggaatcgctagtaatcgtggatcagcatgccacggtgaatacgttctcgggtcttgtacacgccgcccgtcacaccatgggagcttcgctccgatcgaagtcaagttacccttgaccacatcttggcaagtgaccga

SEQ ID NO: 23 - DNA, 1504 bp, Wolbachia sp. wPip
aaatttgagagtttgatcctggctcagaatgaacgctggcggcaggcctaacacatgcaagtcgaacggagttatattgtagcttgctatggtataacttagtggcagacgggtgagtaatgtataggaatctacctagtagtacggaataattgttggaaacgacaactaataccgtatacgccctacgggggaaaaatttattgctattagatgagcctatattagattagctagttggtggggtaatagcctaccaaggtaatgatctatagctgatctgagaggatgatcagccacactggaactgagatacggtccagactcctacgggaggcagcagtggggaatattggacaatgggcgaaagcctgatccagccatgccgcatgagtgaagaaggcctttgggttgtaaagctcttttagtgaggaagataatgacggtactcacagaagaagtcctggctaactccgtgccagcagccgcggtaatacggagagggctagcgttattcggaattattgggcgtaaagggcgcgtaggctggttaataagttaaaagtgaaatcccgaggcttaaccttggaattgcttttaaaactattaatctagagattgaaagaggatagaggaattcctgatgtagaggtaaaattcgtaaatattaggaggaacaccagtggcgaaggcgtctatctggttcaaatctgacgctgaagcgcgaaggcgtggggagcaaacaggattagataccctggtagtccacgctgtaaacgatgaatgttaaatatggggagtttactttctgtattacagctaacgcgttaaacattccgcctggggactacggtcgcaagattaaaactcaaaggaattgacggggacccgcacaagcggtggagcatgtggtttaattcgatgcaacgcgaaaaaccttaccacttcttgacatgaaaatcatacctattcgaagggatagggtcggttcggccggattttacacaagtgttgcatggctgtcgtcagctcgtgtcgtgagatgttgggttaagtcccgcaacgagcgcaaccctcatccttagttgccatcaggtaatgctgagtactttaaggaaactgccagtgataagctggaggaaggtggggatgatgtcaagtcatcatggcctttatggagtgggctacacacgtgctacaatggtgtctacaatgggctgcaaggtgcgcaagcctaagctaatccctaaaagacatctcagttcggattgtactctgcaactcgagtacatgaagttggaatcgctagtaatcgtggatcagcatgccacggtgaatacgttctcgggtcttgtacacactgcccgtcacgccatgggaattggtttcactcgaagctaatggcctaaccgcaaggaaggagttatttaaagtgggatcagtgactggggtgaagtcgtaacaaggtagcagtaggggaatctgcagctggattacctcctta

SEQ ID NO: 24 - DNA, 1532 bp, Uzinura diaspidicola
aaaggagatattccaaccacaccttccggtacggttaccttgttacgacttagccctagtcatcaagtttaccttaggcagaccactgaaggattactgacttcaggtacccccgactcccatggcttgacgggcggtgtgtacaaggttcgagaacatattcaccgcgccattgctgatgcgcgattactagcgattcctgcttcatagagtcgaattgcagactccaatccgaactgagactggttttagagattagctcctgatcacccagtggctgccctttgtaaccagccattgtagcacgtgtgtagcccaaggcatagaggccatgatgatttgacatcatccccaccttcctcacagtttacaccggcagttttgttagagtccccggctttacccgatggcaactaacaataggggttgcgctcgttataggacttaaccaaacacttcacagcacgaactgaagacaaccatgcagcaccttgtaatacgtcgtatagactaagctgtttccagcttattcgtaatacatttaagccttggtaaggttcctcgcgtatcatcgaattaaaccacatgctccaccgcttgtgcgaacccccgtcaattcctttgagtttcaatcttgcgactgtacttcccaggtggatcacttatcgctttcgctaagccactgaatatcgtttttccaatagctagtgatcatcgtttagggcgtggactaccagggtatctaatcctgtttgctccccacgctttcgtgcactgagcgtcagtaaagatttagcaacctgccttcgctatcggtgttctgtatgatatctatgcatttcaccgctacaccatacattccagatgctccaatcttactcaagtttaccagtatcaatagcaattttacagttaagctgtaagctttcactactgacttaataaacagcctacacaccctttaaacccaataaatccgaataacgcttgtgtcatccgtattgccgcggctgctggcacggaattagccgacacttattcgtatagtaccttcaatctcctatcacgtaagatattttatttctatacaaaagcagtttacaacctaaaagaccttcatcctgcacgcgacgtagctggttcagagtttcctccattgaccaatattcctcactgctgcctcccgtaggagtctggtccgtgtctcagtaccagtgtggaggtacaccctcttaggccccctactgatcatagtcttggtagagccattacctcaccaactaactaatcaaacgcaggctcatcttttgccacctaagttttaataaaggctccatgcagaaactttatattatgggggattaatcagaatttcttctggctataccccagcaaaaggtagattgcatacgtgttactcacccattcgccggtcgccgacaaattaaaaatttttcgatgcccctcgacttgcatgtgttaagctcgccgctagcgttaattctgagccaggatcaaactcttcgttgtag

SEQ ID NO: 25 - DNA, 1470 bp, Carsonella ruddii
ctcaggataaacgctagcggagggcttaacacatgcaagtcgaggggcagcaaaaataattatttttggcgaccggcaaacgggtgagtaatacatacgtaactttccttatgctgaggaatagcctgaggaaacttggattaatacctcataatacaattttttagaaagaaaaattgttaaagttttattatggcataagataggcgtatgtccaattagttagttggtaaggtaatggcttaccaagacgatgattggtagggggcctgagaggggcgttcccccacattggtactgagacacggaccaaacttctacggaaggctgcagtgaggaatattggtcaatggaggaaactctgaaccagccactccgcgtgcaggatgaaagaaagccttattggttgtaaactgcttttgtatatgaataaaaaattctaattatagaaataattgaaggtaatatacgaataagtatcgactaactctgtgccagcagtcgcggtaagacagaggatacaagcgttatccggatttattgggtttaaagggtgcgtaggcggtttttaaagtcagtagtgaaatcttaaagcttaactttaaaagtgctattgatactgaaaaactagagtaaggttggagtaactggaatgtgtggtgtagcggtgaaatgcatagatatcacacagaacaccgatagcgaaagcaagttactaaccctatactgacgctgagtcacgaaagcatggggagcaaacaggattagataccctggtagtccatgccgtaaacgatgatcactaactattgggttttatacgttgtaattcagtggtgaagcgaaagtgttaagtgatccacctgaggagtacgaccgcaaggttgaaactcaaaggaattgacgggggcccgcacaatcggtggagcatgtggtttaattcgatgatacacgaggaaccttaccaagacttaaatgtactacgaataaattggaaacaatttagtcaagcgacggagtacaaggtgctgcatggttgtcgtcagctcgtgccgtgaggtgtaaggttaagtcctttaaacgagcgcaacccttattattagttgccatcgagtaatgtcaggggactctaataagactgccggcgcaagccgagaggaaggtggggatgacgtcaaatcatcacggcccttacgtcttgggccacacacgtgctacaatgatcggtacaaaagggagcgactgggtgaccaggagcaaatccagaaagccgatctaagttcggattggagtctgaaactcgactccatgaagctggaatcgctagtaatcgtgcatcagccatggcacggtgaatatgttcccgggccttgtacacaccgcccgtcaagccatggaagttggaagtacctaaagttggttcgctacctaaggtaagtctaataactggggctaagtcgtaacaaggta

SEQ ID NO: 26 - DNA, 1761 bp, Symbiotaphrina buchneri voucher JCM9740
  misc_feature (30)..(30): n is a, g, c, or t
  misc_feature (40)..(40): n is a, g, c, or t
agattaagccatgcaagtctaagtataagnaatctatacngtgaaactgcgaatggctcattaaatcagttatcgtttatttgatagtaccttactacatggataaccgtggtaattctagagctaatacatgctaaaaaccccgacttcggaaggggtgtatttattagataaaaaaccaatgcccttcggggctccttggtgattcatgataacttaacgaatcgcatggccttgcgccggcgatggttcattcaaatttctgccctatcaactttcgatggtaggatagtggcctaccatggttttaacgggtaacggggaattagggttcgattccggagagggagcctgagaaacggctaccacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgcagttaaaaagctcgtagttgaaccttgggcctggctggccggtccgcctaaccgcgtgtactggtccggccgggcctttccttctggggagccgcatgcccttcactgggtgtgtcggggaaccaggacttttactttgaaaaaattagagtgttcaaagcaggcctatgctcgaatacattagcatggaataatagaataggacgtgcggttctattttgttggtttctaggaccgccgtaatgattaatagggatagtcgggggcatcagtattcaattgtcagaggtgaaattcttggatttattgaagactaactactgcgaaagcatttgccaaggatgttttcattaatcagtgaacgaaagttaggggatcgaagacgatcagataccgtcgtagtcttaaccataaactatgccgactagggatcgggcgatgttattattttgactcgctcggcaccttacgagaaatcaaagtctttgggttctggggggagtatggtcgcaaggctgaaacttaaagaaattgacggaagggcaccaccaggagtggagcctgcggcttaatttgactcaacacggggaaactcaccaggtccagacacattaaggattgacagattgagagctctttcttgattatgtgggtggtggtgcatggccgttcttagttggtggagtgatttgtctgcttaattgcgataacgaacgagaccttaacctgctaaatagcccggtccgctttggcgggccgctggcttcttagagggactatcggctcaagccgatggaagtttgaggcaataacaggtctgtgatgcccttagatgttctgggccgcacgcgcgctacactgacagagccaacgagtaaatcaccttggccggaaggtctgggtaatcttgttaaactctgtcgtgctggggatagagcattgcaattattgctcttcaacgaggaattcctagtaagcgcaagtcatcagcttgcgctgattacgtccctgccctttgtacacaccgcccgtcgctactaccgattgaatggctcagtgaggccttcggactggcacagggacgttggcaacgacgacccagtgccggaaagttggtcaaacttggtcatttagaggaagtaaaagtcgtaacaaggtttccgtaggtgaacctgcggaaggatcatta

SEQ ID NO: 27 - DNA, 1801 bp, Symbiotaphrina kochii voucher CBS 589.63
  misc_feature (1753)..(1755): n is a, g, c, or t
tacctggttgattctgccagtagtcatatgcttgtctcaaagattaagccatgcaagtctaagtataagcaatctatacggtgaaactgcgaatggctcattaaatcagttatcgtttatttgatagtaccttactacatggataaccgtggtaattctagagctaatacatgctaaaaacctcgacttcggaaggggtgtatttattagataaaaaaccaatgcccttcggggctccttggtgattcatgataacttaacgaatcgcatggccttgcgccggcgatggttcattcaaatttctgccctatcaactttcgatggtaggatagtggcctaccatggtttcaacgggtaacggggaattagggttcgattccggagagggagcctgagaaacggctaccacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgcagttaaaaagctcgtagttgaaccttgggcctggctggccggtccgcctaaccgcgtgtactggtccggccgggcctttccttctggggagccgcatgcccttcactgggtgtgtcggggaaccaggacttttactttgaaaaaattagagtgttcaaagcaggcctatgctcgaatacattagcatggaataatagaataggacgtgtggttctattttgttggtttctaggaccgccgtaatgattaatagggatagtcgggggcatcagtattcaattgtcagaggtgaaattcttggatttattgaagactaactactgcgaaagcatttgccaaggatgttttcattaatcagtgaacgaaagttaggggatcgaagacgatcagataccgtcgtagtcttaaccataaactatgccgactagggatcgggcgatgttattattttgactcgctcggcaccttacgagaaatcaaagtctttgggttctggggggagtatggtcgcaaggctgaaacttaaagaaattgacggaagggcaccaccaggagtggagcctgcggcttaatttgactcaacacggggaaactcaccaggtccagacacattaaggattgacagattgagagctctttcttgattatgtgggtggtggtgcatggccgttcttagttggtggagtgatttgtctgcttaattgcgataacgaacgagaccttaacctgctaaatagcccggtccgctttggcgggccgctggcttcttagagggactatcggctcaagccgatggaagtttgaggcaataacaggtctgtgatgcccttagatgttctgggccgcacgcgcgctacactgacagagccaacgagtacatcaccttggccggaaggtctgggtaatcttgttaaactctgtcgtgctggggatagagcattgcaattattgctcttcaacgaggaattcctagtaagcgcaagtcatcagcttgcgctgattacgtccctgccctttgtacacaccgcccgtcgctactaccgattgaatggctcagtgaggccttcggactggcacagggacgttggcaacgacgacccagtgccggaaagttcgtcaaacttggtcatttagaggaagnnnaagtcgtaacaaggtttccgtaggtgaacctgcggaaggatcatta

SEQ ID NO: 28 - DNA, 1490 bp, Burkholderia sp. SFA1
agtttgatcctggctcagattgaacgctggcggcatgccttacacatgcaagtcgaacggcagcacgggggcaaccctggtggcgagtggcgaacgggtgagtaatacatcggaacgtgtcctgtagtgggggatagcccggcgaaagccggattaataccgcatacgacctaagggagaaagcgggggatcttcggacctcgcgctataggggcggccgatggcagattagctagttggtggggtaaaggcctaccaaggcgacgatctgtagctggtctgagaggacgaccagccacactgggactgagacacggcccagactcctacgggaggcagcagtggggaattttggacaatgggggcaaccctgatccagcaatgccgcgtgtgtgaagaaggcttcgggttgtaaagcacttttgtccggaaagaaaacttcgtccctaatatggatggaggatgacggtaccggaagaataagcaccggctaactacgtgccagcagccgcggtaatacgtagggtgcgagcgttaatcggaattactgggcgtaaagcgtgcgcaggcggtctgttaagaccgatgtgaaatccccgggcttaacctgggaactgcattggtgactggcaggctttgagtgtggcagaggggggtagaattccacgtgtagcagtgaaatgcgtagagatgtggaggaataccgatggcgaaggcagccccctgggccaactactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaaccctgctgaaaggtgggggtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggttgccaagccgcgaggtggagccaatcccagaaaaccgatcgtagtccggatcgcagtctgcaactcgactgcgtgaagctggaatcgctagtaatcgcggatcagcatgccgcggtgaatacgttcccgggtcttgtacacaccgcccgtcacaccatgggagtgggtttcaccagaagtaggtagcctaaccgcaaggagggcgcttaccacggtgggattcatgactggggtgaagtcgtaacaaggtagc

SEQ ID NO: 29 - DNA, 1408 bp, Burkholderia sp. KM-A
gcaaccctggtggcgagtggcgaacgggtgagtaatacatcggaacgtgtcctgtagtgggggatagcccggcgaaagccggattaataccgcatacgatctacggaagaaagcgggggatccttcgggacctcgcgctataggggcggccgatggcagattagctagttggtggggtaaaggcctaccaaggcgacgatctgtagctggtctgagaggacgaccagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaaacgtcttggttaatacctgaggcggatgacggtaccggaagaataagcaccggctaactacgtgccagcagccgcggtaatacgtagggtgcgagcgttaatcggaattactgggcgtaaagcgtgcgcaggcggtctgttaagaccgatgtgaaatccccgggcttaacctgggaactgcattggtgactggcaggctttgagtgtggcagaggggggtagaattccacgtgtagcagtgaaatgcgtagagatgtggaggaataccgatggcgaaggcagccccctgggccaacactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaagtctgctgagaggtggacgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggttgccaagccgcgaggtggagccaatcccagaaaaccgatcgtagtccggatcgcagtctgcaactcgactgcgtgaagctggaatcgctagtaatcgcggatcagcatgccgcggtgaatacgttcccgggtcttgtacacaccgcccgtcacaccatgggagtgggtttcaccagaagtaggtagcctaaccgcaaggagggcgcttaccacggtgggattcatgactggggtgaagt

SEQ ID NO: 30 - DNA, 1383 bp, Burkholderia sp. KM-G
gcaaccctggtggcgagtggcgaacgggtgagtaatacatcggaacgtgtcctgtagtgggggatagcccggcgaaagccggattaataccgcatacgacctaagggagaaagcgggggatcttcggacctcgcgctataggggcggccgatggcagattagctagttggtggggtaaaggcctaccaaggcgacgatctgtagctggtctgagaggacgaccagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaaacttcgaggttaatacccttggaggatgacggtaccggaagaataagcaccggctaactacgtgccagcagccgcggtaatacgtagggtgcgagcgttaatcggaattactgggcgtaaagcgtgcgcaggcggtctgttaagaccgatgtgaaatccccgggcttaacctgggaactgcattggtgactggcaggctttgagtgtggcagaggggggtagaattccacgtgtagcagtgaaatgcgtagagatgtggaggaataccgatggcgaaggcagccccctgggccaacactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaagtctgctgagaggtggacgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggttgccaagccgcgaggtggagccaatcccagaaaaccgatcgtagtccggatcgcagtctgcaactcgactgcgtgaagctggaatcgctagtaatcgcggatcagcatgccgcggtgaatacgttcccgggtcttgtacacaccgcccgtcacaccatgggagtgggtttcaccagaagtaggtagcctaacctgcaaaggagggcgcttaccacg

SEQ ID NO: 31-32 - skipped (2 entries)

SEQ ID NO: 33 - DNA, 1505 bp, Snodgrassella alvi
gagagtttgatcctggctcagattgaacgctggcggcatgccttacacatgcaagtcgaacggcagcacggagagcttgctctctggtggcgagtggcgaacgggtgagtaatgcatcggaacgtaccgagtaatgggggataactgtccgaaaggatggctaataccgcatacgccctgagggggaaagcgggggatcgaaagacctcgcgttatttgagcggccgatgttggattagctagttggtggggtaaaggcctaccaaggcgacgatccatagcgggtctgagaggatgatccgccacattgggactgagacacggcccaaactcctacgggaggcagcagtggggaattttggacaatggggggaaccctgatccagccatgccgcgtgtctgaagaaggccttcgggttgtaaaggacttttgttagggaagaaaagccgggtgttaataccatctggtgctgacggtacctaaagaataagcaccggctaactacgtgccagcagccgcggtaatacgtagggtgcgagcgttaatcggaattactgggcgtaaagcgagcgcagacggttaattaagtcagatgtgaaatccccgagctcaacttgggacgtgcatttgaaactggttaactagagtgtgtcagagggaggtagaattccacgtgtagcagtgaaatgcgtagagatgtggaggaataccgatggcgaaggcagcctcctgggataacactgacgttcatgctcgaaagcgtgggtagcaaacaggattagataccctggtagtccacgccctaaacgatgacaattagctgttgggacactagatgtcttagtagcgaagctaacgcgtgaaattgtccgcctggggagtacggtcgcaagattaaaactcaaaggaattgacggggacccgcacaagcggtggatgatgtggattaattcgatgcaacgcgaagaaccttacctggtcttgacatgtacggaatctcttagagataggagagtgccttcgggaaccgtaacacaggtgctgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcctcatggcccttatgaccagggcttcacacgtcatacaatggtcggtacagagggtagcgaagccgcgaggtgaagccaatctcagaaagccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaattgggtagactaaccgcaaggaggtcgcttaacacggtatgcttcatgactggggtgaagtcgtaacaaggtagccgtag

SEQ ID NO: 34 - DNA, 1541 bp, Gilliamella apicola
ttaaattgaagagtttgatcatggctcagattgaacgctggcggcaggcttaacacatgcaagtcgaacggtaacatgagtgcttgcacttgatgacgagtggcggacgggtgagtaaagtatggggatctgccgaatggagggggacaacagttggaaacgactgctaataccgcataaagttgagagaccaaagcatgggaccttcgggccatgcgccatttgatgaacccatatgggattagctagttggtagggtaatggcttaccaaggcgacgatctctagctggtctgagaggatgaccagccacactggaactgagacacggtccagactcctacgggaggcagcagtggggaatattgcacaatgggggaaaccctgatgcagccatgccgcgtgtatgaagaaggccttcgggttgtaaagtactttcggtgatgaggaaggtggtgtatctaataggtgcatcaattgacgttaattacagaagaagcaccggctaactccgtgccagcagccgcggtaatacggagggtgcgagcgttaatcggaatgactgggcgtaaagggcatgtaggcggataattaagttaggtgtgaaagccctgggctcaacctaggaattgcacttaaaactggttaactagagtattgtagaggaaggtagaattccacgtgtagcggtgaaatgcgtagagatgtggaggaataccggtggcgaaggcggccttctggacagatactgacgctgagatgcgaaagcgtggggagcaaacaggattagataccctggtagtccacgctgtaaacgatgtcgatttggagtttgttgcctagagtgatgggctccgaagctaacgcgataaatcgaccgcctggggagtacggccgcaaggttaaaactcaaatgaattgacgggggcccgcacaagcggtggagcatgtggtttaattcgatgcaacgcgaagaaccttacctggtcttgacatccacagaatcttgcagagatgcgggagtgccttcgggaactgtgagacaggtgctgcatggctgtcgtcagctcgtgttgtgaaatgttgggttaagtcccgcaacgagcgcaacccttatcctttgttgccatcggttaggccgggaactcaaaggagactgccgttgataaagcggaggaaggtggggacgacgtcaagtcatcatggcccttacgaccagggctacacacgtgctacaatggcgtatacaaagggaggcgacctcgcgagagcaagcggacctcataaagtacgtctaagtccggattggagtctgcaactcgactccatgaagtcggaatcgctagtaatcgtgaatcagaatgtcacggtgaatacgttcccgggccttgtacacaccgcccgtcacaccatgggagtgggttgcaccagaagtagatagcttaaccttcgggagggcgtttaccacggtgtggtccatgactggggtgaagtcgtaacaaggtaaccgtaggggaacctgcggttggatcacctccttac

SEQ ID NO: 35 - DNA, 1528 bp, Bartonella apis
aagccaaaatcaaattttcaacttgagagtttgatcctggctcagaacgaacgctggcggcaggcttaacacatgcaagtcgaacgcacttttcggagtgagtggcagacgggtgagtaacgcgtgggaatctacctatttctacggaataacgcagagaaatttgtgctaataccgtatacgtccttcgggagaaagatttatcggagatagatgagcccgcgttggattagctagttggtgaggtaatggcccaccaaggcgacgatccatagctggtctgagaggatgaccagccacattgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggatttactgggcgtaaagcgcacgtaggcggatatttaagtcaggggtgaaatcccggggctcaaccccggaactgcctttgatactggatatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgctgtaaacgatgaatgttagccgttggacagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccgatcgcggatggtggagacaccgtctttcagttcggctggatcggtgacaggtgctgcatggctgtcgtcagctcgtgtcgtgagatgttgggttaagtcccgcaacgagcgcaaccctcgcccttagttgccatcatttagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgtggatcagcatgccacggtgaatacgttcccgggccttgtacacaccgcccgtcacaccatgggagttggttttacccgaaggtgctgtgctaaccgcaaggaggcaggcaaccacggtagggtcagcgactggggtgaagtcgtaacaaggtagccgtaggggaacctgcggctggatcacctcctttctaaggaagatgaagaattggaa

SEQ ID NO: 36 - DNA, 1390 bp, Parasaccharibacter apium
  misc_feature (643)..(756): n is a, g, c, or t
ctaccatgcaagtcgcacgaaacctttcggggttagtggcggacgggtgagtaacgcgttaggaacctatctggaggtgggggataacatcgggaaactggtgctaataccgcatgatgcctgagggccaaaggagagatccgccattggaggggcctgcgttcgattagctagttggttgggtaaaggctgaccaaggcgatgatcgatagctggtttgagaggatgatcagccacactgggactgagacacggcccagactcctacgggaggcagcagtggggaatattggacaatgggggcaaccctgatccagcaatgccgcgtgtgtgaagaaggtcttcggattgtaaagcactttcactagggaagatgatgacggtacctagagaagaagccccggctaacttcgtgccagcagccgcggtaatacgaagggggctagcgttgctcggaatgactgggcgtaaagggcgcgtaggctgtttgtacagtcagatgtgaaatccccgggcttaacctgggaactgcatttgatacgtgcagactagagtccgagagagggttgtggaattcccagtgtagaggtgaaattcgtagatattgggaagaacaccggttgcgaaggcggcaacctggctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagctaacgcgttaagcacaccgcctggggagtacggccgcaaggttgaaactcaaaggaattgacgggggcccgcacaagcggtggagcatgtggtttaattcgaagcaacgcgcagaaccttaccagggcttgcatggggaggctgtattcagagatggatatttcttcggacctcccgcacaggtgctgcatggctgtcgtcagctcgtgtcgtgagatgttgggttaagtcccgcaacgagcgcaacccttgtctttagttgccatcacgtctgggtgggcactctagagagactgccggtgacaagccggaggaaggtggggatgacgtcaagtcctcatggcccttatgtcctgggctacacacgtgctacaatggcggtgacagagggatgctacatggtgacatggtgctgatctcaaaaaaccgtctcagttcggattgtactctgcaactcgagtgcatgaaggtggaatcgctagtaatcgcggatcagcatgccgcggtgaatacgttcccgggccttgtacacaccgcccgtcacaccatgggagttggtttgaccttaagccggtgagcgaaccgcaaggaacgcagccgaccaccggttcgggttcagcgactggggga

SEQ ID NO: 37 - DNA, 1583 bp, Lactobacillus kunkeei
ttccttagaaaggaggtgatccagccgcaggttctcctacggctaccttgttacgacttcaccctaatcatctgtcccaccttagacgactagctcctaaaaggttaccccatcgtctttgggtgttacaaactctcatggtgtgacgggcggtgtgtacaaggcccgggaacgtattcaccgtggcatgctgatccacgattactagtgattccaacttcatgcaggcgagttgcagcctgcaatccgaactgagaatggctttaagagattagcttgacctcgcggtttcgcgactcgttgtaccatccattgtagcacgtgtgtagcccagctcataaggggcatgatgatttgacgtcgtccccaccttcctccggtttatcaccggcagtctcactagagtgcccaactaaatgctggcaactaataataagggttgcgctcgttgcgggacttaacccaacatctcacgacacgagctgacgacaaccatgcaccacctgtcattctgtccccgaagggaacgcccaatctcttgggttggcagaagatgtcaagagctggtaaggttcttcgcgtagcatcgaattaaaccacatgctccaccacttgtgcgggcccccgtcaattcctttgagtttcaaccttgcggtcgtactccccaggcggaatacttaatgcgttagctgcggcactgaagggcggaaaccctccaacacctagtattcatcgtttacggcatggactaccagggtatctaatcctgttcgctacccatgctttcgagcctcagcgtcagtaacagaccagaaagccgccttcgccactggtgttcttccatatatctacgcatttcaccgctacacatggagttccactttcctcttctgtactcaagttttgtagtttccactgcacttcctcagttgagctgagggctttcacagcagacttacaaaaccgcctgcgctcgctttacgcccaataaatccggacaacgcttgccacctacgtattaccgcggctgctggcacgtagttagccgtggctttctggttaaataccgtcaaagtgttaacagttactctaacacttgttcttctttaacaacagagttttacgatccgaaaaccttcatcactcacgcggcgttgctccatcagactttcgtccattgtggaagattccctactgctgcctcccgtaggagtctgggccgtgtctcagtcccaatgtggccgattaccctctcaggtcggctacgtatcatcgtcttggtgggcttttatctcaccaactaactaatacggcgcgggtccatcccaaagtgatagcaaagccatctttcaagttggaaccatgcggttccaactaattatgcggtattagcacttgtttccaaatgttatcccccgcttcggggcaggttacccacgtgttactcaccagttcgccactcgctccgaatccaaaaatcatttatgcaagcataaaatcaatttgggagaactcgttcgacttgcatgtattaggcacgccgccagcgttcgtcctgagccaggatcaaactctcatcttaa

SEQ ID NO: 38 - DNA, 1395 bp, Lactobacillus Firm-4
acgaacgctggcggcgtgcctaatacatgcaagtcgagcgcgggaagtcagggaagccttcgggtggaactggtggaacgagcggcggatgggtgagtaacacgtaggtaacctgccctaaagcgggggataccatctggaaacaggtgctaataccgcataaacccagcagtcacatgagtgctggttgaaagacggcttcggctgtcactttaggatggacctgcggcgtattagctagttggtggagtaacggttcaccaaggcaatgatacgtagccgacctgagagggtaatcggccacattgggactgagacacggcccaaactcctacgggaggcagcagtagggaatcttccacaatggacgcaagtctgatggagcaacgccgcgtggatgaagaaggtcttcggatcgtaaaatcctgttgttgaagaagaacggttgtgagagtaactgctcataacgtgacggtaatcaaccagaaagtcacggctaactacgtgccagcagccgcggtaatacgtaggtggcaagcgttgtccggatttattgggcgtaaagggagcgcaggcggtcttttaagtctgaatgtgaaagccctcagcttaactgaggaagagcatcggaaactgagagacttgagtgcagaagaggagagtggaactccatgtgtagcggtgaaatgcgtagatatatggaagaacaccagtggcgaaggcggctctctggtctgttactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctcctgcaagcctaagagattaggggttcccttcggggacaggaagacaggtggtgcatggttgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcaaatcatcatgccccttatgacctgggctacacacgtgctacaatggatggtacaatgagaagcgaactcgcgaggggaagctgatctctgaaaaccattctcagttcggattgcaggctgcaactcgcctgcatgaagctggaatcgctagtaatcgcggatcagcatgccgcggtgaatacgttcccgggccttgtacacaccgccc

SEQ ID NO: 39 - DNA, 1549 bp, Enterococcus
aggtgatccagccgcaccttccgatacggctaccttgttacgacttcaccccaatcatctatcccaccttaggcggctggctccaaaaaggttacctcaccgacttcgggtgttacaaactctcgtggtgtgacgggcggtgtgtacaaggcccgggaacgtattcaccgcggcgtgctgatccgcgattactagcgattccggcttcatgcaggcgagttgcagcctgcaatccgaactgagagaagctttaagagatttgcatgacctcgcggtctagcgactcgttgtacttcccattgtagcacgtgtgtagcccaggtcataaggggcatgatgatttgacgtcatccccaccttcctccggtttgtcaccggcagtctcgctagagtgcccaactaaatgatggcaactaacaataagggttgcgctcgttgcgggacttaacccaacatctcacgacacgagctgacgacaaccatgcaccacctgtcactttgtccccgaagggaaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgtttgctgcagcactgaagggcggaaaccctccaacacttagcactcatcgtttacggcgtggactaccagggtatctaatcctgtttgctccccacgctttcgagcctcagcgtcagttacagaccagagagccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaggggacgttcagttactaacgtccttgttcttctctaacaacagagttttacgatccgaaaaccttcttcactcacgcggcgttgctcggtcagactttcgtccattgccgaagattccctactgctgcctcccgtaggagtctgggccgtgtctcagtcccagtgtggccgatcaccctctcaggtcggctatgcatcgtggccttggtgagccgttacctcaccaactagctaatgcaccgcgggtccatccatcagcgacacccgaaagcgcctttcactcttatgccatgcggcataaactgttatgcggtattagcacctgtttccaagtgttatccccctctgatgggtaggttacccacgtgttactcacccgtccgccactcctctttccaattgagtgcaagcactcgggaggaaagaagcgttcgacttgcatgtattaggcacgccgccagcgttcgtcctgagccaggatcaaactct

SEQ ID NO: 40 - DNA, 1541 bp, Delftia
cagaaaggaggtgatccagccgcaccttccgatacggctaccttgttacgacttcaccccagtcacgaaccccgccgtggtaagcgccctccttgcggttaggctacctacttctggcgagacccgctcccatggtgtgacgggcggtgtgtacaagacccgggaacgtattcaccgcggcatgctgatccgcgattactagcgattccgacttcacgcagtcgagttgcagactgcgatccggactacgactggttttatgggattagctccccctcgcgggttggcaaccctctgtaccagccattgtatgacgtgtgtagccccacctataagggccatgaggacttgacgtcatccccaccttcctccggtttgtcaccggcagtctcattagagtgctcaactgaatgtagcaactaatgacaagggttgcgctcgttgcgggacttaacccaacatctcacgacacgagctgacgacagccatgcagcacctgtgtgcaggttctctttcgagcacgaatccatctctggaaacttcctgccatgtcaaaggtgggtaaggtttttcgcgttgcatcgaattaaaccacatcatccaccgcttgtgcgggtccccgtcaattcctttgagtttcaaccttgcggccgtactccccaggcggtcaacttcacgcgttagcttcgttactgagaaaactaattcccaacaaccagttgacatcgtttagggcgtggactaccagggtatctaatcctgtttgctccccacgctttcgtgcatgagcgtcagtacaggtccaggggattgccttcgccatcggtgttcctccgcatatctacgcatttcactgctacacgcggaattccatccccctctaccgtactctagccatgcagtcacaaatgcagttcccaggttgagcccggggatttcacatctgtcttacataaccgcctgcgcacgctttacgcccagtaattccgattaacgctcgcaccctacgtattaccgcggctgctggcacgtagttagccggtgcttattcttacggtaccgtcatgggccccctgtattagaaggagctttttcgttccgtacaaaagcagtttacaacccgaaggccttcatcctgcacgcggcattgctggatcaggctttcgcccattgtccaaaattccccactgctgcctcccgtaggagtctgggccgtgtctcagtcccagtgtggctggtcgtcctctcagaccagctacagatcgtcggcttggtaagcttttatcccaccaactacctaatctgccatcggccgctccaatcgcgcgaggcccgaagggcccccgctttcatcctcagatcgtatgcggtattagctactctttcgagtagttatcccccacgactgggcacgttccgatgtattactcacccgttcgccactcgtcagcgtccgaagacctgttaccgttcgacttgcatgtgtaaggcatgccgccagcgttcaatctgagccaggatcaaactctacagttcgatct

SEQ ID NO: 41 - DNA, 1502 bp, Pelomonas
  misc_feature (192)..(193): n is a, g, c, or t
  misc_feature (832)..(833): n is a, g, c, or t
atcctggctcagattgaacgctggcggcatgccttacacatgcaagtcgaacggtaacaggttaagctgacgagtggcgaacgggtgagtaatatatcggaacgtgcccagtcgtgggggataactgctcgaaagagcagctaataccgcatacgacctgagggtgaaagcgggggatcgcaagacctcgcnngattggagcggccgatatcagattaggtagttggtggggtaaaggcccaccaagccaacgatctgtagctggtctgagaggacgaccagccacactgggactgagacacggcccagactcctacgggaggcagcagtggggaattttggacaatgggcgcaagcctgatccagccatgccgcgtgcgggaagaaggccttcgggttgtaaaccgcttttgtcagggaagaaaaggttctggttaatacctgggactcatgacggtacctgaagaataagcaccggctaactacgtgccagcagccgcggtaatacgtagggtgcaagcgttaatcggaattactgggcgtaaagcgtgcgcaggcggttatgcaagacagaggtgaaatccccgggctcaacctgggaactgcctttgtgactgcatagctagagtacggtagagggggatggaattccgcgtgtagcagtgaaatgcgtagatatgcggaggaacaccgatggcgaaggcaatcccctggacctgtactgacgctcatgcacgaaagcgtggggagcaaacaggattagataccctggtagtccacgccctaaacgatgtcaactggttgttgggagggtttcttctcagtaacgtanntaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgccaggaatcctgaagagatttgggagtgctcgaaagagaacctggacacaggtgctgcatggccgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaggtcatcatggcccttatgggtagggctacacacgtcatacaatggccgggacagagggctgccaacccgcgagggggagctaatcccagaaacccggtcgtagtccggatcgtagtctgcaactcgactgcgtgaagtcggaatcgctagtaatcgcggatcagcttgccgcggtgaatacgttcccgggtcttgtacacaccgcccgtcacaccatgggagcgggttctgccagaagtagttagcctaaccgcaaggagggcgattaccacggcagggttcgtgactggggtgaagtcgtaacaaggtagccgtatcggaaggtgcggctggatcac

SEQ ID NO: 42 - protein, 34 aa, Lactococcus lactis
ITSISLCTPGCKTGALMGCNMKTATCHCSIHVSK

SEQ ID NO: 43 - protein, 22 aa, Staphylococcus epidermis
IASKFICTPGCAKTGSFNSYCC

SEQ ID NO: 44 - protein, 44 aa, Pediococcus acidilactici
KYYGNGVTCGKHSCSVDWGKATTCIINNGAMAWATGGHQGNHKC

SEQ ID NO: 45 - protein, 44 aa, Enterococcus faecium
ATRSYGNGVYCNNSKCWVNWGEAKENIAGIVISGWASGLAGMGH

SEQ ID NO: 46 - protein, 39 aa, Streptococcus lactis
GTWDDIGQGIGRVAYWVGKAMGNMSDVNQASRINRKKKH

SEQ ID NO: 47 - protein, 48 aa, Lactobacillus johnsonii
NRWGDTVLSAASGAGTGIKACKSFGPWGMAICGVGGAAIGGYFGYTHN

SEQ ID NO: 48 - protein, 70 aa, Enterococcus faecalis
MAKEFGIPAAVAGTVLNVVEAGGWVTTIVSILTAVGSGGLSLLAAAGRESIKAYLKKEIKKKGKRAVIAW

SEQ ID NO: 49 - protein, 51 aa, Staphylococcus aureus [sequence omitted]

SEQ ID NO: 50 - protein, 43 aa, Lactococcus garvieae
IGGALGNALNGLGTWANMMNGGGFVNQWQVYANKGKINQYRPY

SEQ ID NO: 51 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 52 - protein, 339 aa, Cp1
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 53 - protein, 296 aa, Dp-1
MGVDIEKGVAWMQARKGRVSYSMDFRDGPDSYDCSSSMYYALRSAGASSAGWAVNTEYMHAWLIENGYELISENAPWDAKRGDIFIWGRKGASAGAGGHTGMFIDSDNIIHCNYAYDGISVNDHDERWYYAGQPYYYVYRLTNANAQPAEKKLGWQKDATGFWYARANGTYPKDEFEYIEENKSWFYFDDQGYMLAEKWLKHTDGNWYWFDRDGYMATSWKRIGESWYYFNRDGSMVTGWIKYYDNWYYCDATNGDMKSNAFIRYNDGWYLLLPDGRLADKPQFTVEPDGLITAKV

SEQ ID NO: 54 - protein, 233 aa, gamma
MEIQKKLVDPSKYGTKCPYTMKPKYITVHNTYNDAPAENEVSYMISNNNEVSFHIAVDDKKAIQGIPLERNAWACGDGNGSGNRQSISVEICYSKSGGDRYYKAEDNAVDVVRQLMSMYNIPIENVRTHQSWSGKYCPHRMLAEGRWGAFIQKVKNGNVATTSPTKQNIIQSGAFSPYETPDVMGALTSLKMTADFILQSDGLTYFISKPTSDAQLKAMKEYLDRKGWWYEVK

SEQ ID NO: 55 - protein, 481 aa, MR11
MQAKLTKKEFIEWLKTSEGKQFNVDLWYGFQCFDYANAGWKVLFGLLLKGLGAKDIPFANNFDGLATVYQNTPDFLAQPGDMVVFGSNYGAGYGHVAWVIEATLDYIIVYEQNWLGGGWTDRIEQPGWGWEKVTRRQHAYDFPMWFIRPNFKSETAPRSIQSPTQASKKETAKPQPKAVELKIIKDVVKGYDLPKRGGNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKQLQLKDYFIKQIRVYMDGKIPVATVSNESSASSNTVKPVASAWKRNKYGTYYMEENARFTNGNQPITVRKIGPFLSCPVAYQFQPGGYCDYTEVMLQDGHVWVGYTWEGQRYYLPIRTWNGSAPPNQILGDLWGEIS

SEQ ID NO: 56 - protein, 239 aa, B30
MVINIEQAIAWMASRKGKVTYSMDYRNGPSSYDCSSSVYFALRSAGASDNGWAVNTEYEHDWLIKNGYVLIAENTNWNAQRGDIFIWGKRGASAGAFGHTGMFVDPDNIIHCNYGYNSITVNNHDEIWGYNGQPYVYAYRYSGKQSNAKVDNKSVVSKFEKELDVNTPLSNSNMPYYEATISEDYYVESKPDVNSTDKELLVAGTRVRVYEKVKGWARIGAPQSNQWVEDAYLIDATDM

SEQ ID NO: 57 - protein, 495 aa, K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 58 - protein, 281 aa, A118
MTSYYYSRSLANVNKLADNTKAAARKLLDWSESNGIEVLIYETIRTKEQQAANVNSGASQTMRSYHLVGQALDFVMAKGKTVDWGAYRSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 59 - protein, 341 aa, A511
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKK

SEQ ID NO: 60 - protein, 289 aa, A500
MALTEAWLIEKANRKLNAGGMYKITSDKTRNVIKKMAKEGIYLCVAQGYRSTAEQNALYAQGRTKPGAIVTNAKGGQSNHNYGVAVDLCLYTNDGKDVIWESTTSRWKKVVAAMKAEGFKWGGDWKSFKDYPHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 61 - protein, 239 aa, LambdaSa1 prophage
MVINIEQAIAWMASRKGKVTYSMDYRNGPSSYDCSSSVYFALRSAGASDNGWAVNTEYEHDWLIKNGYVLIAENTNWNAQRGDIFIWGKRGASAGAFGHTGMFVDPDNIIHCNYGYNSITVNNHDEIWGYNGQPYVYAYRYARKQSNAKVDNQSVVSKFEKELDVNTPLSNSNMPYYEATISEDYYVESKPDVNSTDKELLVAGTRVRVYEKVKGWARIGAPQSNQWVEDAYLIDATDM

SEQ ID NO: 62 - protein, 468 aa, LambdaSa2 prophage
MEINTEIAIAWMSARQGKVSYSMDYRDGPNSYDCSSSVYYALRSAGASSAGWAVNTEYMHDWLIKNGYELIAENVDWNAVRGDIAIWGMRGHSSGAGGHVVMFIDPENIIHCNWANNGITVNNYNQTAAASGWMYCYVYRLKSGASTQGKSLDTLVKETLAGNYGNGEARKAVLGNQYEAVMSVINGKTTTNQKTVDQLVQEVIAGKHGNGEARKKSLGSQYDAVQKRVTELLKKQPSEPFKAQEVNKPTETKTSQTELTGQATATKEEGDLSFNGTILKKAVLDKILGNCKKHDILPSYALTILHYEGLWGTSAVGKADNNWGGMTWTGQGNRPSGVTVTQGSARPSNEGGHYMHYASVDDFLTDWFYLLRAGGSYKVSGAKTFSEAIKGMFKVGGAVYDYAASGFDSYIVGASSRLKAIEAENGSLDKFDKATDIGDGSKDKIDITIEGIEVTINGITYELTKKPV

SEQ ID NO: 63 - protein, 236 aa, (ATCC700407) prophage
MTDSIQEMRKLQSIPVRYDMGDRYGNDADRDGRIEMDCSSAVSKALGISMTNNTETLQQALPAIGYGKIHDAVDGTFDMQAYDVIIWAPRDGSSSLGAFGHVLIATSPTTAIHCNYGSDGITENDYNYIWEINGRPREIVFRKGVTQTQATVTSQFERELDVNARLTVSDKPYYEATLSEDYYVEAGPRIDSQDKELIKAGTRVRVYEKLNGWSRINHPESAQWVEDSYLVDATEM

SEQ ID NO: 64 - protein, 481 aa, Phi11 and Phi12
MQAKLTKNEFIEWLKTSEGKQFNVDLWYGFQCFDYANAGWKVLFGLLLKGLGAKDIPFANNFDGLATVYQNTPDFLAQPGDMVVFGSNYGAGYGHVAWVIEATLDYIIVYEQNWLGGGWTDGIEQPGWGWEKVTRRQHAYDFPMWFIRPNFKSETAPRSVQSPTQAPKKETAKPQPKAVELKIIKDVVKGYDLPKRGSNPKGIVIHNDAGSKGATAEAYRNGLVNAPLSRLEAGIAHSYVSGNTVWQALDESQVGWHTANQIGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNESSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 65 - protein, 481 aa, (Phi)H5
MQAKLTKKEFIEWLKTSEGKQYNADGWYGFQCFDYANAGWKALFGLLLKGVGAKDIPFANNFDGLATVYQNTPDFLAQPGDMVVFGSNYGAGYGHVAWVIEATLDYIIVYEQNWLGGGWTDGVQQPGSGWEKVTRRQHAYDFPMWFIRPNFKSETAPRSVQSPTQASKKETAKPQPKAVELKIIKDVVKGYDLPKRGSNPNFIVIHNDAGSKGATAEAYRNGLVNAPLSRLEAGIAHSYVSGNTVWQALDESQVGWHTANQIGNKYGYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 66 - protein, 477 aa, (Phi)WMY
MKTKAQAKSWINSKIGKGIDWDGMYGYQCMDEAVDYIHHVTDGKVTMWGNAIDAPKNNFQGLCTVYTNTPEFRPAYGDVIVWSYGTFATYGHIAIVVNPDPYGDLQYITVLEQNWNGNGIYKTEFATIRTHDYTGVSHFIRPKFADEVKETAKTVNKLSVQKKIVTPKNSVERIKNYVKTSGYINGEHYELYNRGHKPKGVVIHNTAGTASATQEGQRLTNMTFQQLANGVAHVYIDKNTIYETLPEDRIAWHVAQQYGNTEFYGIEVCGSRNTDKEQFLANEQVAFQEAARRLKSWGMRANRNTVRLHHTFSSTECPDMSMLLHTGYSMKNGKPTQDITNKCADYFMKQINAYIDGKQPTSTVVGSSSSNKLKAKNKDKSTGWNTNEYGTLWKKEHATFTCGVRQGIVTRTTGPFTSCPQAGVLYYGQSVNYDTVCKQDGYVWISWTTSDGYDVWMPIRTWDRSTDKVSEIWGTIS

SEQ ID NO: 67 - protein, 443 aa, (Phi)NCTC 11261
MATYQEYKSRSNGNAYDIDGSFGAQCWDGYADYCKYLGLPYANCTNTGYARDIWEQRHENGILNYFDEVEVMQAGDVAIFMVVDGVTPYSHVAIFDSDAGGGYGWFLGQNQGGANGAYNIVKIPYSATYPTAFRPKVFKNAVTVTGNIGLNKGDYFIDVSAYQQADLTTTCQQAGTTKTIIKVSESIAWLSDRHQQQANTSDPIGYYHFGRFGGDSALAQREADLFLSNLPSKKVSYLVIDYEDSASADKQANTNAVIAFMDKIASAGYKPIYYSYKPFTLNNIDYQKIIAKYPNSIWIAGYPDYEVRTEPLWEFFPSMDGVRWWQFTSVGVAGGLDKNIVLLADDSSKMDIPKVDKPQELTFYQKLATNTKLDNSNVPYYEATLSTDYYVESKPNASSADKEFIKAGTRVRVYEKVNGWSRINHPESAQWVEDSYLVNATDM

SEQ ID NO: 68 - protein, 334 aa, (Phi)FWLLm3
MVKYTVENKIIAGLPKGKLKGANFVIAHETANSKSTIDNEVSYMTRNWQNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPSTNLDKLGLVDYMNAKKMDSSYSNRAKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKK

SEQ ID NO: 69 - protein, 1278 aa, (Phi)BPS13
MAKREKYIFDVEAEVGKAAKSIKSLEAELSKLQKLNKEIDATGGDRTEKEMLATLKAAKEVNAEYQKMQRILKDLSKYSGKVSRKEFNDSKVINNAKTSVQGGKVTDSFGQMLKNMERQINSVNKQFDNHRKAMVDRGQQYTPHLKTNRKDSQGNSNPSMMGRNKSTTQDMEKAVDKFLNGQNEATTGLNQALYQLKEISKLNRRSESLSRRASASGYMSFQQYSNFTGDRRTVQQTYGGLKTANRERVLELSGQATGISKELDRLNSKKGLTAREGEERKKLMRQLEGIDAELTARKKLNSSLDETTSNMEKFNQSLVDAQVSVKPERGTMRGMMYERAPAIALAIGGAITATIGKLYSEGGNHSKAMRPDEMYVGQQTGAVGANWRPNRTATMRSGLGNHLGFTGQEMMEFQSNYLSANGYHGAEDMKAATTGQATFARATGLGSDEVKDFFNTAYRSGGIDGNQTKQFQNAFLGAMKQSGAVGREKDQLKALNGILSSMSQNRTVSNQDMMRTVGLQSAISSSGVASLQGTKGGALMEQLDNGIREGFNDPQMRVLFGQGTKYQGMGGRAALRKQMEKGISDPDNLNTLIDASKASAGQDPAEQAEVLATLASKMGVNMSSDQARGLIDLQPSGKLTKENIDKVMKEGLKEGSIESAKRDKAYSESKASIDNSSEAATAKQATELNDMGSKLRQANAALGGLPAPLYTAIAAVVAFTAAVAGSALMFKGASWLKGGMASKYGGKGGKGGKGGGTGGGGGAGGAAATGAGAAAGAGGVGAAAAGEVGAGVAAGGAAAGAAAGGSKLAGVGKGFMKGAGKLMLPLGILMGASEIMQAPEEAKGSAIGSAVGGIGGGIAGGAATGAIAGSFLGPIGTAVGGIAGGIAGGFAGSSLGETIGGWFDSGPKEDASAADKAKADASAAALAAAAGTSGAVGSSALQSQMAQGITGAPNMSQVGSMASALGISSGAMASALGISSGQENQIQTMTDKENTNTKKANEAKKGDNLSYERENISMYERVLTRAEQILAQARAQNGIMGVGGGGTAGAGGGINGFTGGGKLQFLAAGQKWSSSNLQQHDLGFTDQNLTAEDLDKWIDSKAPQGSMMRGMGATFLKAGQEYGLDPRYLIAHAAEESGWGTSKIARDKGNFFGIGAFDDSPYSSAYEFKDGTGSAAERGIMGGAKWISEKYYGKGNTTLDKMKAAGYATNASWAPNIASIMAGAPTGSGSGNVTATINVNVKGDEKVSDKLKNSSDMKKAGKDIGSLLGFYSREMTIA

SEQ ID NO: 70 - protein, 495 aa, (Phi)GH15
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGDRVYCPVRTCQGVPPNHIPGVAWGVFK

SEQ ID NO: 71 - protein, 264 aa, (Phi)8074-B1
MKIGIDMGHTLSGADYGVVGLRPESVLTREVGTKVIYKLQKLGHVVVNCTVDKASSVSESLYTRYYRANQANVDLFISIHFNATPGGTGTEVYTYAGRQLGEATRIRQEFKSLGLRDRGTKDGSGLAVIRNTKAKAMLVECCFCDNPNDMKLYNSESFSNAIVKGITGKLPNGESGNNNQGGNKVKAVVIYNEGADRRGAEYLADYLNCPTISNSRTFDYSCVEHVYAVGGKKEQYTKYLKTLLSGANRYDTMQQILNFINGGK

SEQ ID NO: 72 - protein, 209 aa, (Phi)SPN1S
MDINQFRRASGINEQLAARWFPHITTAMNEFGITKPDDQAMFIAQVGHESGGFTRLQENFNYSVNGLSGFIRAGRITPDQANALGRKTYEKSLPLERQRAIANLVYSKRMGNNGPGDGWNYRGRGLIQITGLNNYRDCGNGLKVDLVAQPELLAQDEYAARSAAWFFSSKGCMKYTGDLVRVTQIINGGQNGIDDRRTRYAAARKVLAL

SEQ ID NO: 73 - protein, 290 aa, (Phi)CN77
MGYWGYPNGQIPNDKMALYRGCLLRADAAAQAYALQDAYTRATGKPLVILEGYRDLTRQKYLRNLYLSGRGNIAAVPGLSNHGWGLACDFAAPLNSSGSEEHRWMRQNAPLFGFDWARGKADNEPWHWEYGNVPVSRWASLDVTPIDRNDMADITEGQMQRIAVILLDTEIQTPLGPRLVKHALGDALLLGQANANSIAEVPDKTWDVLVDHPLAKNEDGTPLKVRLGDVAKYEPLEHQNTRDAIAKLGTLQFTDKQLATIGAGVKPIDEASLVKKIVDGVRALFGRAAA

SEQ ID NO: 74 - protein, 185 aa, (Phi)AB2
MILTKDGFSIIRNELFGGKLDQTQVDAINFIVAKATESGLTYPEAAYLLATIYHETGLPSGYRTMQPIKEAGSDSYLRSKKYYPYIGYGYVQLTWKENYERIGKLIGVDLIKNPEKALEPLIAIQIAIKGMLNGWFTGVGFRRKRPVSKYNKQQYVAARNIINGKDKAELIAKYAIIFERALRSL

SEQ ID NO: 75 - protein, 262 aa, (Phi)B4
MAMALQTLIDKANRKLNVSGMRKDVADRTRAVITQMHAQGIYICVAQGFRSFAEQNALYAQGRTKPGSIVTNARGGQSNHNYGVAVDLCLYTQDGSDVIWTVEGNFRKVIAAMKAQGFKWGGDWVSFKDYPHFELYDVVGGQKPPADNGGAVDNGGGSGSTGGSGGGSTGGGSTGGGYDSSWFTKETGTFVTNTSIKLRTAPFTSADVIATLPAGSPVNYNGFGIEYDGYVWIRQPRSNGYGYLATGESKGGKRQNYWGTFK

SEQ ID NO: 76 - protein, 274 aa, (Phi)CTP1
MKKIADISNLNGNVDVKLLFNLGYIGIIAKASEGGTFVDKYYKQNYTNTKAQGKITGAYHFANFSTIAKAQQEANFFLNCIAGTTPDFVVLDLEQQCTGDITDACLAFLNIVAKKFKCVVYCNSSFIKEHLNSKICAYPLWIANYGVATPAFTLWTKYAMWQFTEKGQVSGISGYIDFSYITDEFIKYIKGEDEVENLVVYNDGADQRAAEYLADRLACPTINNARKFDYSNVKNVYAVGGNKEQYTSYLTTLIAGSTRYTTMQAVLDYIKNLK

SEQ ID NO: 77 - protein, 628 aa, Staphylococcus virus 187
MALPKTGKPTAKQVVDWAINLIGSGVDVDGYYGRQCWDLPNYIFNRYWNFKTPGNARDMAWYRYPEGFKVFRNTSDFVPKPGDIAVWTGGNYNWNTWGHTGIVVGPSTKSYFYSVDQNWNNSNSYVGSPAAKIKHSYFGVTHFVRPAYKAEPKPTPPAQNNPAPKDPEPSKKPESNKPIYKVVTKILFTTAHIEHVKANRFVHYITKSDNHNNKPNKIVIKNTNTALSTIDVYRYRDELDKDEIPHFFVDRLNVWACRPIEDSINGYHDSVVLSITETRTALSDNFKMNEIECLSLAESILKANNKKMSASNIIVDNKAWRTFKLHTGKDSLKSSSFTSKDYQKAVNELIKLFNDKDKLLNNKPKDVVERIRIRTIVKENTKFVPSELKPRNNIRDKQDSKIDRVINNYTLKQALNIQYKLNPKPQTSNGVSWYNASVNQIKSAMDTTKIFNNNVQVYQFLKLNQYQGIPVDKLNKLLVGKGTLANQGHAFADGCKKYNINEIYLIAHRFLESANGTSFFASGKTGVYNYFGIGAFDNNPNNAMAFARSHGWTSPTKAIIGGAEFVGKGYFNVGQNTLYRMRWNPQKPGTHQYATDISWAKVQAQMISAMYKEIGLTGDYFIYDQYKK

SEQ ID NO: 78 - protein, 291 aa, (Phi)P35
MARKFTKAELVAKAEKKVGGLKPDVKKAVLSAVKEAYDRYGIGIIVSQGYRSIAEQNGLYAQGRTKPGNIVTNAKGGQSNHNFGVAVDFAIDLIDDGKIDSWQPSATIVNMMKRRGFKWGGDWKSFTDLPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 79 - protein, 342 aa, (Phi)CP-7
MVKKNDLFVDVASHQGYDISGILEEAGTTNTIIKVSESTSYLNPCLSAQVSQSNPIGFYHFAWFGGNEEEAEAEARYFLDNVPTQVKYLVLDYEDHASASVQRNTTACLRFMQIIAEAGYTPIYYSYKPFTLDNVDYQQILAQFPNSLWIAGYGLNDGTANFEYFPSMDGIRWWQYSSNPFDKNIVLLDDEKEDNINNENTLKSLTTVANEVIQGLWGNGQERYDSLANAGYDPQAVQDKVNEILNAREIADLTTVANEVIQGLWGNGQERYDSLANAGYDPQAVQDKVNEILNAREIADLTTVANEVIQGLWGNGQERYDSLANAGYDPQAVQDKVNELLS

SEQ ID NO: 80 - protein, 328 aa, (Phi)EFAP-1
MKLKGILLSVVTTFGLLFGATNVQAYEVNNEFNLQPWEGSQQLAYPNKIILHETANPRATGRNEATYMKNNWFNAHTTAIVGDGGIVYKVAPEGNVSWGAGNANPYAPVQIELQHTNDPELFKANYKAYVDYTRDMGKKFGIPMTLDQGGSLWEKGVVSHQWVTDFVWGDHTDPYGYLAKMGISKAQLAHDLANGVSGNTATPTPKPDKPKPTQPSKPSNKKRFNYRVDGLEYVNGMWQIYNEHLGKIDFNWTENGIPVEVVDKVNPATGQPTKDQVLKVGDYFNFQENSTGVVQEQTPYMGYTLSHVQLPNEFIWLFTDSKQALMYQ

SEQ ID NO: 81 - protein, 48 aa, Homo sapiens
SSLLEKGLDGAKKAVGGLGKLGKDAVEDLESVGKGAVHDVKDVLDSVL

SEQ ID NO: 82 - protein, 37 aa, Hyalophora cecropia
KWKLFKKIEKVGQNIRDGIIKAGPAVAVVGQATQIAK

SEQ ID NO: 83 - protein, 62 aa, Drosophila teissieri
MKYFSVLVVLTLILAIVDQSDAFINLLDKVEDALHTGAQAGFKLIRPVERGATPKKSEKPEK

SEQ ID NO: 84 - protein, 66 aa, Bombyz mori
MNILKFFFVFIVAMSLVSCSTAAPAKIPIKAIKTVGKAVGKGLRAINIASTANDVFNFLKPKKRKH

SEQ ID NO: 85 - protein, 71 aa, Ceratitis capitata
MANLKAVFLICIVAFIALQCVVAEPAAEDSVVVKRSIGSALKKALPVAKKIGKIALPIAKAALPVAAGLVG

SEQ ID NO: 86 - protein, 53 aa, Apis mellifera
MKVVIFIFALLATICAAFAYVPLPNVPQPGRRPFPTFPGQGPFNPKIKWPQGY

SEQ ID NO: 87 - protein, 283 aa, Apis mellifera
KNFALAILVVTFVVAVFGNTNLDPPTRPTRLRREAKPEAEPGNNRPVYIPQPRPPHPRLRREAEPEAEPGNNRPVYIPQPRPPHPRLRREAELEAEPGNNRPVYISQPRPPHPRLRREAEPEAEPGNNRPVYIPQPRPPHPRLRREAELEAEPGNNRPVYISQPRPPHPRLRREAEPEAEPGNNRPVYIPQPRPPHPRLRREAEPEAEPGNNRPVYIPQPRPPHPRLRREAEPEAEPGNNRPVYIPQPRPPHPRLRREAKPEAKPGNNRPVYIPQPRPPHPRI

SEQ ID NO: 88 - protein, 228 aa, Sus scrofa
METQRASLCLGRWSLWLLLLALVVPSASAQALSYREAVLRAVDRLNEQSSEANLYRLLELDQPPKADEDPGTPKPVSFTVKETVCPRPTRRPPELCDFKENGRVKQCVGTVTLDQIKDPLDITCNEGVRRFPWWWPFLRRPRLRRQAFPPPNVPGPRFPPPNVPGPRFPPPNFPGPRFPPPNFPGPRFPPPNFPGPPFPPPIFPGPWFPPPPPFRPPPFGPPRFPGRR

SEQ ID NO: 89 - protein, 144 aa, Bos taurus
MQTQRASLSLGRWSLWLLLLGLVVPSASAQALSYREAVLRAVDQLNELSSEANLYRLLELDPPPKDNEDLGTRKPVSFTVKETVCPRTIQQPAEQCDFKEKGRVKQCVGTVTLDPSNDQFDLNCNELQSVILPWKWPWWPWRRG

SEQ ID NO: 90 - protein, 149 aa, Sus scrofa
METQRASLCLGRWSLWLLLLALVVPSASAQALSYREAVLRAVDRLNEQSSEANLYRLLELDQPPKADEDPGTPKPVSFTVKETVCPRPTRQPPELCDFKENGRVKQCVGTVTLDQIKDPLDITCNEVQGVRGGRLCYCRRRFCVCVGRG

SEQ ID NO: 91 - protein, 17 aa, Tachypleus gigas
KWCFRVCYRGICYRRCR

SEQ ID NO: 92 - protein, 102 aa, Anopheles gambiae
MKCATIVCTIAVVLAATLLNGSVQAAPQEEAALSGGANLNTLLDELPEETHHAALENYRAKRATCDLASGFGVGSSLCAAHCIARRYRGGYCNSKAVCVCRN

SEQ ID NO: 93 - protein, 70 aa, Drosophila melanogaster
MMQIKYLFALFAVLMLVVLGANEADADCLSGRYKGPCAVWDNETCRRVCKEEGRSSGHCSPSLKCWCEGC

SEQ ID NO: 94 - protein, 63 aa, Medicago truncatula
MTKIVVFIYVVILLLTIFHVSAKKKRYIECETHEDCSQVFMPPFVMRCVIHECKIFNGEHLRY

SEQ ID NO: 95 - protein, 76 aa, Medicago truncatula
MAKIMKFVYNMIPFLSIFIITLQVNVVVCEIDADCPQICMPPYEVRCVNHRCGWVNTDDSLFLTQEFTRSKQYIIS

SEQ ID NO: 96 - protein, 76 aa, Medicago truncatula
MYKVVESIFIRYMHRKPNMTKFFKFVYTMFILISLFLVVTNANAHNCTDISDCSSNHCSYEGVSLCMNGQCICIYE

SEQ ID NO: 97 - protein, 64 aa, Medicago truncatula
MVETLRLFYIMILFVSLCLVVVDGESKLEQTCSEDFECYIKNPHVPFGHLRCFEGFCQQLNGPA

SEQ ID NO: 98 - protein, 67 aa, Medicago truncatula
MAKIVNFVYSMIVFLFLFLVATKAARGYLCVTDSHCPPHMCPPGMEPRCVRRMCKCLPIGWRKYFVP

SEQ ID NO: 99 - protein, 96 aa, Medicago truncatula
MQIGKNMVETPKLDYVIIFFFLYFFFRQMIILRLNTTFRPLNFKMLRFWGQNRNIMKHRGQKVHFSLILSDCKTNKDCPKLRRANVRCRKSYCVPI

SEQ ID NO: 100 - protein, 65 aa, Medicago truncatula
MLRLYLVSYFLLKRTLLVSYFSYFSTYIIECKTDNDCPISQLKIYAWKCVKNGCHLFDVIPMMYE

SEQ ID NO: 101 - protein, 79 aa, Medicago truncatula
MAEILKFVYIVILFVSLLLIVVASERECVTDDDCEKLYPTNEYRMMCDSGYCMNLLNGKIIYLLCLKKKKFLIIISVLL

SEQ ID NO: 102 - protein, 95 aa, Medicago truncatula
MAEIIKFVYIMILCVSLLLIEVAGEECVTDADCDKLYPDIRKPLMCSIGECYSLYKGKFSLSIISKTSFSLMVYNVVTLVICLRLAYISLLLKFL

SEQ ID NO: 103 - protein, 100 aa, Medicago truncatula
MAEILKDFYAMNLFIFLIILPAKIRGETLSLTHPKCHHIMLPSLFITEVFQRVTDDGCPKPVNHLRVVKCIEHICEYGYNYRPDFASQIPESTKMPRKRE

SEQ ID NO: 104 - protein, 78 aa, Medicago truncatula
MVEILKNFYAMNLFIFLIILAVKIRGAHFPCVTDDDCPKPVNKLRVIKCIDHICQYARNLPDFASEISESTKMPCKGE

SEQ ID NO: 105 - protein, 72 aa, Medicago truncatula
MFHAQAENMAKVSNFVCIMILFLALFFITMNDAARFECREDSHCVTRIKCVLPRKPECRNYACGCYDSNKYR

SEQ ID NO: 106 - protein, 78 aa, Medicago truncatula
MQMRQNMATILNFVFVIILFISLLLVVTKGYREPFSSFTEGPTCKEDIDCPSISCVNPQVPKCIMFECHCKYIPTTLK

SEQ ID NO: 107 - protein, 71 aa, Medicago truncatula
MATILMYVYITILFISILTVLTEGLYEPLYNFRRDPDCRRNIDCPSYLCVAPKVPRCIMFECHCKDIPSDH

SEQ ID NO: 108 - protein, 57 aa, Medicago truncatula
MTTSLKFVYVAILFLSLLLVVMGGIRRFECRQDSDCPSYFCEKLTVPKCFWSKCYCK

SEQ ID NO: 109 - protein, 57 aa, Medicago truncatula
MTTSLKFVYVAILFLSLLLVVMGGIRKKECRQDSDCPSYFCEKLTIAKCIHSTCLCK

SEQ ID NO: 110 - protein, 66 aa, Medicago truncatula
MQIGKNMVETPKLVYFIILFLSIFLCITVSNSSFSQIFNSACKTDKDCPKFGRVNVRCRKGNCVPI

SEQ ID NO: 111 - protein, 57 aa, Medicago truncatula
MTAILKKFINAVFLFIVLFLATTNVEDFVGGSNDECVYPDVFQCINNICKCVSHHRT

SEQ ID NO: 112 - protein, 74 aa, Medicago truncatula
MQKRKNMAQIIFYVYALIILFSPFLAARLVFVNPEKPCVTDADCDRYRHESAIYSDMFCKDGYCFIDYHHDPYP

SEQ ID NO: 113 - protein, 76 aa, Medicago truncatula
MQMRKNMAQILFYVYALLILFTPFLVARIMVVNPNNPCVTDADCQRYRHKLATRMICNQGFCLMDFTHDPYAPSLP

SEQ ID NO: 114 - protein, 64 aa, Medicago truncatula
MNHISKFVYALIIFLSIYLVVLDGLPISCKDHFECRRKINILRCIYRQEKPMCINSICTCVKLL

SEQ ID NO: 115 - protein, 67 aa, Medicago truncatula
MQREKNMAKIFEFVYAMIIFILLFLVEKNVVAYLKFECKTDDDCQKSLLKTYVWKCVKNECYFFAKK

SEQ ID NO: 116 - protein, 61 aa, Medicago truncatula
MAGIIKFVHVLIIFLSLFHVVKNDDGSFCFKDSDCPDEMCPSPLKEMCYFLQCKCGVDTIA

SEQ ID NO: 117 - protein, 59 aa, Medicago truncatula
MANTHKLVSMILFIFLFLASNNVEGYVNCETDADCPPSTRVKRFKCVKGECRWTRMSYA

SEQ ID NO: 118 - protein, 63 aa, Medicago truncatula
MQRRKKKAQVVMFVHDLIICIYLFIVITTRKTDIRCRFYYDCPRLEYHFCECIEDFCAYIRLN

SEQ ID NO: 119 - protein, 57 aa, Medicago truncatula
MAKVYMFVYALIIFVSPFLLATFRTRLPCEKDDDCPEAFLPPVMKCVNRFCQYEILE

SEQ ID NO: 120 - protein, 77 aa, Medicago truncatula
MIKQFSVCYIQMRRNMTTILKFPYIMVICLLLLHVAAYEDFEKEIFDCKKDGDCDHMCVTPGIPKCTGYVCFCFENL

SEQ ID NO: 121 - protein, 73 aa, Medicago truncatula
MQRSRNMTTIFKFAYIMIICVFLLNIAAQEIENGIHPCKKNEDCNHMCVMPGLPWCHENNLCFCYENAYGNTR

SEQ ID NO: 122 - protein, 85 aa, Medicago truncatula
MTIIIKFVNVLIIFLSLFHVAKNDDNKLLLSFIEEGFLCFKDSDCPYNMCPSPLKEMCYFIKCVCGVYGPIRERRLYQSHNPMIQ

SEQ ID NO: 123 - protein, 69 aa, Medicago truncatula
MRKNMTKILMIGYALMIFIFLSIAVSITGNLARASRKKPVDVIPCIYDHDCPRKLYFLERCVGRVCKYL

SEQ ID NO: 124 - protein, 58 aa, Medicago truncatula
MAHKLVYAITLFIFLFLIANNIEDDIFCITDNDCPPNTLVQRYRCINGKCNLSFVSYG

SEQ ID NO: 125 - protein, 61 aa, Medicago truncatula
MDETLKFVYILILFVSLCLVVADGVKNINRECTQTSDCYKKYPFIPWGKVRCVKGRCRLDM

SEQ ID NO: 126 - protein, 62 aa, Medicago truncatula
MAKIIKFVYVLAIFFSLFLVAKNVNGWTCVEDSDCPANICQPPMQRMCFYGECACVRSKFCT

SEQ ID NO: 127 - protein, 61 aa, Medicago truncatula
MVKIIKFVYFMTLFLSMLLVTTKEDGSVECIANIDCPQIFMLPFVMRCINFRCQIVNSEDT

SEQ ID NO: 128 - protein, 67 aa, Medicago truncatula
MDEILKFVYTLIIFFSLFFAANNVDANIMNCQSTFDCPRDMCSHIRDVICIFKKCKCAGGRYMPQVP

SEQ ID NO: 129 - protein, 62 aa, Medicago truncatula
MQRRKNMANNHMLIYAMIICLFPYLVVTFKTAITCDCNEDCLNFFTPLDNLKCIDNVCEVFM

SEQ ID NO: 130 - protein, 65 aa, Medicago truncatula
MVNILKFIYVIIFFILMFFVLIDVDGHVLVECIENRDCEKGMCKFPFIVRCLMDQCKCVRIHNLI

SEQ ID NO: 131 - protein, 74 aa, Medicago truncatula
MIIQFSIYYMQRRKLNMVEILKFSHALIIFLFLSALVTNANIFFCSTDEDCTWNLCRQPWVQKCRLHMCSCEKN

SEQ ID NO: 132 - protein, 58 aa, Medicago truncatula
MDEVFKFVYVMIIFPFLILDVATNAEKIRRCFNDAHCPPDMCTLGVIPKCSRFTICIC

SEQ ID NO: 133 - protein, 64 aa, Medicago truncatula
MHRKPNMTKFFKFVYTMFILISLFLVVTNANANNCTDTSDCSSNHCSYEGVSLCMNGQCICIYE

SEQ ID NO: 134 - protein, 69 aa, Medicago truncatula
MQMKKMATILKFVYLIILLIYPLLVVTEESHYMKFSICKDDTDCPTLFCVLPNVPKCIGSKCHCKLMVN

SEQ ID NO: 135 - protein, 64 aa, Medicago truncatula
MVETLRLFYIMILFVSLYLVVVDGVSKLAQSCSEDFECYIKNPHAPFGQLRCFEGYCQRLDKPT

SEQ ID NO: 136 - protein, 63 aa, Medicago truncatula
MTTFLKVAYIMIICVFVLHLAAQVDSQKRLHGCKEDRDCDNICSVHAVTKCIGNMCRCLANVK

SEQ ID NO: 137 - protein, 61 aa, Medicago truncatula
MRINRTPAIFKFVYTIIIYLFLLRVVAKDLPFNICEKDEDCLEFCAHDKVAKCMLNICFCF

SEQ ID NO: 138 - protein, 54 aa, Medicago truncatula
MAEILKILYVFIIFLSLILAVISQHPFTPCETNADCKCRNHKRPDCLWHKCYCY

SEQ ID NO: 139 - protein, 65 aa, Medicago truncatula
MRKSMATILKFVYVIMLFIYSLFVIESFGHRFLIYNNCKNDTECPNDCGPHEQAKCILYACYCVE

SEQ ID NO: 140 - protein, 58 aa, Medicago truncatula
MNTILKFIFVVFLFLSIFLSAGNSKSYGPCTTLQDCETHNWFEVCSCIDFECKCWSLL

SEQ ID NO: 141 - protein, 64 aa, Medicago truncatula
MAEIIKFVYIMILCVSLLLIAEASGKECVTDADCENLYPGNKKPMFCNNTGYCMSLYKEPSRYM

SEQ ID NO: 142 - protein, 64 aa, Medicago truncatula
MAKIIKFVYIMILCVSLLLIVEAGGKECVTDVDCEKIYPGNKKPLICSTGYCYSLYEEPPRYHK

SEQ ID NO: 143 - protein, 64 aa, Medicago truncatula
MAKVTKFGYIIIHFLSLFFLAMNVAGGRECHANSHCVGKITCVLPQKPECWNYACVCYDSNKYR

SEQ ID NO: 144 - protein, 55 aa, Medicago truncatula
MAKIFNYVYALIMFLSLFLMGTSGMKNGCKHTGHCPRKMCGAKTTKCRNNKCQCV

SEQ ID NO: 145 - protein, 69 aa, Medicago truncatula
MTEILKFVCVMIIFISSFIVSKSLNGGGKDKCFRDSDCPKHMCPSSLVAKCINRLCRCRRPELQVQLNP

SEQ ID NO: 146 - protein, 69 aa, Medicago truncatula
MAHIIMFVYALIYALIIFSSLFVRDGIPCLSDDECPEMSHYSFKCNNKICEYDLGEMSDDDYYLEMSRE

SEQ ID NO: 147 - protein, 77 aa, Medicago truncatula
MYREKNMAKTLKFVYVIVLFLSLFLAAKNIDGRVSYNSFIALPVCQTAADCPEGTRGRTYKCINNKCRYPKLLKPIQ

SEQ ID NO: 148 - protein, 56 aa, Medicago truncatula
MAHIFNYVYALLVFLSLFLMVTNGIHIGCDKDRDCPKQMCHLNQTPKCLKNICKCV

SEQ ID NO: 149 - protein, 77 aa, Medicago truncatula
MAEILKCFYTMNLFIFLIILPAKIREHIQCVIDDDCPKSLNKLLIIKCINHVCQYVGNLPDFASQIPKSTKMPYKGE

SEQ ID NO: 150 - protein, 70 aa, Medicago truncatula
MAYISRIFYVLIIFLSLFFVVINGVKSLLLIKVRSFIPCQRSDDCPRNLCVDQIIPTCVWAKCKCKNYND

SEQ ID NO: 151 - protein, 64 aa, Medicago truncatula
MANVTKFVYIAIYFLSLFFIAKNDATATFCHDDSHCVTKIKCVLPRTPQCRNEACGCYHSNKFR

SEQ ID NO: 152 - protein, 62 aa, Medicago truncatula
MGEIMKFVYVMIIYLFMFNVATGSEFIFTKKLTSCDSSKDCRSFLCYSPKFPVCKRGICECI

SEQ ID NO: 153 - protein, 61 aa, Medicago truncatula
MGEMFKFIYTFILFVHLFLVVIFEDIGHIKYCGIVDDCYKSKKPLFKIWKCVENVCVLWYK

SEQ ID NO: 154 - protein, 63 aa, Medicago truncatula
MARTLKFVYSMILFLSLFLVANGLKIFCIDVADCPKDLYPLLYKCIYNKCIVFTRIPFPFDWI

SEQ ID NO: 155 - protein, 66 aa, Medicago truncatula
MANITKFVYIAILFLSLFFIGMNDAAILECREDSHCVTKIKCVLPRKPECRNNACTCYKGGFSFHH

SEQ ID NO: 156 - protein, 68 aa, Medicago truncatula
MQRVKKMSETLKFVYVLILFISIFHVVIVCDSIYFPVSRPCITDKDCPNMKHYKAKCRKGFCISSRVR

SEQ ID NO: 157 - protein, 72 aa, Medicago truncatula
MQIRKIMSGVLKFVYAIILFLFLFLVAREVGGLETIECETDGDCPRSMIKMWNKNYRHKCIDGKCEWIKKLP

SEQ ID NO: 158 - protein, 54 aa, Medicago truncatula
MFVYDLILFISLILVVTGINAEADTSCHSFDDCPWVAHHYRECIEGLCAYRILY

SEQ ID NO: 159 - protein, 69 aa, Medicago truncatula
MQRRKKSMAKMLKFFFAIILLLSLFLVATEVGGAYIECEVDDDCPKPMKNSHPDTYYKCVKHRCQWAWK

SEQ ID NO: 160 - protein, 140 aa, Medicago truncatula
MFVYTLIIFLFPSHVITNKIAIYCVSDDDCLKTFTPLDLKCVDNVCEFNLRCKGKCGERDEKFVFLKALKKMDQKLVLEEQGNAREVKIPKKLLFDRIQVPTPATKDQVEEDDYDDDDEEEEEEEDDVDMWFHLPDVVCH

SEQ ID NO: 161 - protein, 60 aa, Medicago truncatula
MAKFSMFVYALINFLSLFLVETAITNIRCVSDDDCPKVIKPLVMKCIGNYCYFFMIYEGP

SEQ ID NO: 162 - protein, 58 aa, Medicago truncatula
MAHKFVYAIILFIFLFLVAKNVKGYVVCRTVDDCPPDTRDLRYRCLNGKCKSYRLSYG

SEQ ID NO: 163 - protein, 61 aa, Medicago truncatula
MQRKKNMGQILIFVFALINFLSPILVEMTTTTIPCTFIDDCPKMPLVVKCIDNFCNYFEIK

SEQ ID NO: 164 - protein, 57 aa, Medicago truncatula
MAQTLMLVYALIIFTSLFLVVISRQTDIPCKSDDACPRVSSHHIECVKGFCTYWKLD

SEQ ID NO: 165 - protein, 77 aa, Medicago truncatula
MLRRKNTVQILMFVSALLIYIFLFLVITSSANIPCNSDSDCPWKIYYTYRCNDGFCVYKSIDPSTIPQYMTDLIFPR

SEQ ID NO: 166 - protein, 59 aa, Medicago truncatula
MAVILKFVYIMIIFLFLLYVVNGTRCNRDEDCPFICTGPQIPKCVSHICFCLSSGKEAY

SEQ ID NO: 167 - protein, 62 aa, Medicago truncatula
MDAILKFIYAMFLFLFLFVTTRNVEALFECNRDFVCGNDDECVYPYAVQCIHRYCKCLKSRN

SEQ ID NO: 168 - protein, 67 aa, Medicago truncatula
MQIGRKKMGETPKLVYVIILFLSIFLCTNSSFSQMINFRGCKRDKDCPQFRGVNIRCRSGFCTPIDS

SEQ ID NO: 169 - protein, 76 aa, Medicago truncatula
MQMRKNMAQILFYVYALLILFSPFLVARIMVVNPNNPCVTDADCQRYRHKLATRMVCNIGFCLMDFTHDPYAPSLP

SEQ ID NO: 170 - protein, 77 aa, Medicago truncatula
MYVYYIQMGKNMAQRFMFIYALIIFLSQFFVVINTSDIPNNSNRNSPKEDVFCNSNDDCPTILYYVSKCVYNFCEYW

SEQ ID NO: 171 - protein, 67 aa, Medicago truncatula
MAKIVNFVYSMIIFVSLFLVATKGGSKPFLTRPYPCNTGSDCPQNMCPPGYKPGCEDGYCNHCYKRW

SEQ ID NO: 172 - protein, 62 aa, Medicago truncatula
MVRTLKFVYVIILILSLFLVAKGGGKKIYCENAASCPRLMYPLVYKCLDNKCVKFMMKSRFV

SEQ ID NO: 173 - protein, 62 aa, Medicago truncatula
MARTLKFVYAVILFLSLFLVAKGDDVKIKCVVAANCPDLMYPLVYKCLNGICVQFTLTFPFV

SEQ ID NO: 174 - protein, 65 aa, Medicago truncatula
MSNTLMFVITFIVLVTLFLGPKNVYAFQPCVTTADCMKTLKTDENIWYECINDFCIPFPIPKGRK

SEQ ID NO: 175 - protein, 76 aa, Medicago truncatula
MKRVVNMAKIVKYVYVIIIFLSLFLVATKIEGYYYKCFKDSDCVKLLCRIPLRPKCMYRHICKCKVVLTQNNYVLT

SEQ ID NO: 176 - protein, 66 aa, Medicago truncatula
MKRGKNMSKILKFIYATLVLYLFLVVTKASDDECKIDGDCPISWQKFHTYKCINQKCKWVLRFHEY

SEQ ID NO: 177 - protein, 64 aa, Medicago truncatula
MAKTLNFMFALILFISLFLVSKNVAIDIFVCQTDADCPKSELSMYTWKCIDNECNLFKVMQQMV

SEQ ID NO: 178 - protein, 59 aa, Medicago truncatula
MANTHKLVSMILFIFLFLVANNVEGYVNCETDADCPPSTRVKRFKCVKGECRWTRMSYA

SEQ ID NO: 179 - protein, 59 aa, Medicago truncatula
MAHFLMFVYALITCLSLFLVEMGHLSIHCVSVDDCPKVEKPITMKCINNYCKYFVDHKL

SEQ ID NO: 180 - protein, 66 aa, Medicago truncatula
MNQIPMFGYTLIIFFSLFPVITNGDRIPCVTNGDCPVMRLPLYMRCITYSCELFFDGPNLCAVERI

SEQ ID NO: 181 - protein, 61 aa, Medicago truncatula
MRKDMARISLFVYALIIFFSLFFVLTNGELEIRCVSDADCPLFPLPLHNRCIDDVCHLFTS

SEQ ID NO: 182 - protein, 60 aa, Medicago truncatula
MAQILMFVYFLIIFLSLFLVESIKIFTEHRCRTDADCPARELPEYLKCQGGMCRLLIKKD

SEQ ID NO: 183 - protein, 56 aa, Medicago truncatula
MARVISLFYALIIFLFLFLVATNGDLSPCLRSGDCSKDECPSHLVPKCIGLTCYCI

SEQ ID NO: 184 - protein, 62 aa, Medicago truncatula
MQRRKNMAQILLFAYVFIISISLFLVVTNGVKIPCVKDTDCPTLPCPLYSKCVDGFCKMLSI

SEQ ID NO: 185 - protein, 66 aa, Medicago truncatula
MNHISKFVYALIIFLSVYLVVLDGRPVSCKDHYDCRRKVKIVGCIFPQEKPMCINSMCTCIREIVP

SEQ ID NO: 186 - protein, 86 aa, Medicago truncatula
MKSQNHAKFISFYKNDLFKIFQNNDSHFKVFFALIIFLYTYLHVTNGVFVSCNSHIHCRVNNHKIGCNIPEQYLLCVNLFCLWLDY

SEQ ID NO: 187 - protein, 62 aa, Medicago truncatula
MTYISKVVYALIIFLSIYVGVNDCMLVTCEDHFDCRQNVQQVGCSFREIPQCINSICKCMKG

SEQ ID NO: 188 - protein, 63 aa, Medicago truncatula
MTHISKFVFALIIFLSIYVGVNDCKRIPCKDNNDCNNNWQLLACRFEREVPRCINSICKCMPM

SEQ ID NO: 189 - protein, 60 aa, Medicago truncatula
MVQTPKLVYVIVLLLSIFLGMTICNSSFSHFFEGACKSDKDCPKLHRSNVRCRKGQCVQI

SEQ ID NO: 190 - protein, 77 aa, Medicago truncatula
MTKILMLFYAMIVFHSIFLVASYTDECSTDADCEYILCLFPIIKRCIHNHCKCVPMGSIEPMSTIPNGVHKFHIINN

SEQ ID NO: 191 - protein, 64 aa, Medicago truncatula
MAKTLNFVCAMILFISLFLVSKNVALYIIECKTDADCPISKLNMYNWRCIKSSCHLYKVIQFMV

SEQ ID NO: 192 - protein, 72 aa, Medicago truncatula
MQKEKNMAKTFEFVYAMIIFILLFLVENNFAAYIIECQTDDDCPKSQLEMFAWKCVKNGCHLFGMYEDDDDP

SEQ ID NO: 193 - protein, 57 aa, Medicago truncatula
MAATRKFIYVLSHFLFLFLVTKITDARVCKSDKDCKDIIIYRYILKCRNGECVKIKI

SEQ ID NO: 194 - protein, 75 aa, Medicago truncatula
MQRLDNMAKNVKFIYVIILLLFIFLVIIVCDSAFVPNSGPCTTDKDCKQVKGYIARCRKGYCMQSVKRTWSSYSR

SEQ ID NO: 195 - protein, 102 aa, Medicago truncatula
MKFIYIMILFLSLFLVQFLTCKGLTVPCENPTTCPEDFCTPPMITRCINFICLCDGPEYAEPEYDGPEPEYDHKGDFLSVKPKIINENMMMRERHMMKEIEV

SEQ ID NO: 196 - protein, 59 aa, Medicago truncatula
MAQFLMFIYVLIIFLYLFYVEAAMFELTKSTIRCVTDADCPNVVKPLKPKCVDGFCEYT

SEQ ID NO: 197 - protein, 70 aa, Medicago truncatula
MKMRIHMAQIIMFFYALIIFLSPFLVDRRSFPSSFVSPKSYTSEIPCKATRDCPYELYYETKCVDSLCTY

SEQ ID NO: 198 - protein, 41 aa, Medicago truncatula
TRMLTIPCTSDDNCPKVISPCHTKCFDGFCGWYIEGSYEGP

SEQ ID NO: 199 - protein, 69 aa, Medicago truncatula
MAQFLLFVYSLIIFLSLFFGEAAFERTETRMLTIPCTSDDNCPKVISPCHTKCFDGFCGWYIEGSYEGP

SEQ ID NO: 200 - protein, 78 aa, Buchnera aphidicola
MKLLHGFLIIMLTMHLSIQYAYGGPFLTKYLCDRVCHKLCGDEFVCSCIQYKSLKGLWFPHCPTGKASVVLHNFLTSP

SEQ ID NO: 201 - protein, 77 aa, Buchnera aphidicola
MKLLYGFLIIMLTIHLSVQYFESPFETKYNCDTHCNKLCGKIDHCSCIQYHSMEGLWFPHCRTGSAAQMLHDFLSNP

SEQ ID NO: 202 - protein, 86 aa, Buchnera aphidicola
MSVRKNVLPTMFVVLLIMSPVTPTSVFISAVCYSGCGSLALVCFVSNGITNGLDYFKSSAPLSTSETSCGEAFDTCTDHCLANFKF

SEQ ID NO: 203 - protein, 69 aa, Buchnera aphidicola
MRLLYGFLIIMLTIYLSVQDFDPTEFKGPFPTIEICSKYCAVVCNYTSRPCYCVEAAKERDQWFPYCYD

SEQ ID NO: 204 - protein, 77 aa, Buchnera aphidicola
MRLLYGFLIIMLTIHLSVQDIDPNTLRGPYPTKEICSKYCEYNVVCGASLPCICVQDARQLDHWFACCYDGGPEMLM

SEQ ID NO: 205 - protein, 108 aa, Buchnera aphidicola
MKLFVVVVLVAVGIMFVFASDTAAAPTDYEDTNDMISLSSLVGDNSPYVRVSSADSGGSSKTSSKNPILGLLKSVIKLLTKIFGTYSDAAPAMPPIPPALRKNRGMLA

SEQ ID NO: 206 - protein, 178 aa, Buchnera aphidicola
MVACKVILAVAVVFVAAVQGRPGGEPEWAAPIFAELKSVSDNITNLVGLDNAGEYATAAKNNLNAFAESLKTEAAVFSKSFEGKASASDVFKESTKNFQAVVDTYIKNLPKDLTLKDFTEKSEQALKYMVEHGTEITKKAQGNTETEKEIKEFFKKQIENLIGQGKALQAKIAEAKKA

SEQ ID NO: 207 - protein, 311 aa, Buchnera aphidicola
MKTSSSKVFASCVAIVCLASVANALPVQKSVAATTENPIVEKHGCRAHKNLVRQNVVDLKTYDSMLITNEVVQKQSNEVQSSEQSNEGQNSEQSNEGQNSEQSNEVQSSEHSNEGQNSKQSNEGQNSEQSNEVQSSEHSNEGQNSEQSNEVQSSEHSNEGQNSKQSNEGQNSKQSNEVQSSEHWNEGQNSKQSNEDQNSEQSNEGQNSKQSNEGQNSKQSNEDQNSEQSNEGQNSKQSNEVQSSEQSNEGQNSKQSNEGQSSEQSNEGQNSKQSNEVQSPEEHYDLPDPESSYESEETKGSHESGDDSEHR

SEQ ID NO: 208 - protein, 431 aa, Buchnera aphidicola
MKTIILGLCLFGALFWSTQSMPVGEVAPAVPAVPSEAVPQKQVEAKPETNAASPVSDAKPESDSKPVDAEVKPTVSEVKAESEQKPSGEPKPESDAKPVVASESKPESDPKPAAVVESKPENDAVAPETNNDAKPENAAAPVSENKPATDAKAETELIAQAKPESKPASDLKAEPEAAKPNSEVPVALPLNPTETKATQQSVETNQVEQAAPAAAQADPAAAPAADPAPAPAAAPVAAEEAKLSESAPSTENKAAEEPSKPAEQQSAKPVEDAVPAASEISETKVSPAVPAVPEVPASPSAPAVADPVSAPEAEKNAEPAKAANSAEPAVQSEAKPAEDIQKSGAVVSAENPKPVEEQKPAEVAKPAEQSKSEAPAEAPKPTEQSAAEEPKKPESANDEKKEQHSVNKRDATKEKKPTDSIMKKQKQKKAN

SEQ ID NO: 209 - protein, 160 aa, Buchnera aphidicola
MNGKIVLCFAVVFIGQAMSAATGTTPEVEDIKKVAEQMSQTFMSVANHLVGITPNSADAQKSIEKIRTIMNKGFTDMETEANKMKDIVRKNADPKLVEKYDELEKELKKHLSTAKDMFEDKVVKPIGEKVELKKITENVIKTTKDMEATMNKAIDGFKKQ

SEQ ID NO: 210 - protein, 415 aa, Buchnera aphidicola
MHLFLALGLFIVCGMVDATFYNPRSQTFNQLMERRQRSIPIPYSYGYHYNPIEPSINVLDSLSEGLDSRINTFKPIYQNVKMSTQDVNSVPRTQYQPKNSLYDSEYISAKDIPSLFPEEDSYDYKYLGSPLNKYLTRPSTQESGIAINLVAIKETSVFDYGFPTYKSPYSSDSVWNFGSKIPNTVFEDPQSVESDPNTFKVSSPTIKIVKLLPETPEQESIITTTKNYELNYKTTQETPTEAELYPITSEEFQTEDEWHPMVPKENTTKDESSFITTEEPLTEDKSNSITIEKTQTEDESNSIEFNSIRTEEKSNSITTEENQKEDDESMSTTSQETTTAFNLNDTFDTNRYSSSHESLMLRIRELMKNIADQQNKSQFRTVDNIPAKSQSNLSSDESTNQQFEPQLVNGADTYK

SEQ ID NO: 211 - protein, 126 aa, Sitophilus zeamais
MTRTMLFLACVAALYVCISATAGKPEEFAKLSDEAPSNDQAMYESIQRYRRFVDGNRYNGGQQQQQQPKQWEVRPDLSRDQRGNTKAQVEINKKGDNHDINAGWGKNINGPDSHKDTWHVGGSVRW

SEQ ID NO: 212 - protein, 220 aa, Acyrthosiphon pisum
MKETTVVWAKLFLILIILAKPLGLKAVNECKRLGNNSCRSHGECCSGFCFIEPGWALGVCKRLGTPKKSDDSNNGKNIEKNNGVHERIDDVFERGVCSYYKGPSITANGDVFDENEMTAAHRTLPFNTMVKVEGMGTSVVVKINDRKTAADGKVMLLSRAAAESLNIDENTGPVQCQLKFVLDGSGCTPDYGDTCVLHHECCSQNCFREMFSDKGFCLPK

SEQ ID NO: 213 - protein, 16 aa, Artificial Sequence
  Penetratin
RQIKIWFQNRRMKWKK

SEQ ID NO: 214 - protein, 13 aa, Human immunodeficiency virus 1
GRKKRRQRRRPPQ

SEQ ID NO: 215 - protein, 18 aa, Artificial Sequence
  pVEC
LLIILRRRIRKQAHAHSK

SEQ ID NO: 216 - protein, 27 aa, Artificial Sequence
  Transportan
GWTLNSAGYLLGKINLKALAALAKKIL

SEQ ID NO: 217 - protein, 27 aa, Artificial Sequence
  MPG
GALFLGFLGAAGSTMGAWSQPKKKRKV

SEQ ID NO: 218 - protein, 21 aa, Artificial Sequence
  Pep-1
KETWWETWWTEWSQPKKKRKV

SEQ ID NO: 219 - protein, 18 aa, Artificial Sequence
  MAP
KLALKLALKALKAALKLA

SEQ ID NO: 220 - protein, 9 aa, Artificial Sequence
  R6W3
RRWWRRWRR

SEQ ID NO: 221 - DNA, 17 bp, Artificial Sequence
  Buchnera forward primer
gtcggctcatcacatcc

SEQ ID NO: 222 - DNA, 20 bp, Artificial Sequence
  Buchnera reverse primer
ttccgtctgtattatctcct

SEQ ID NO: 223 - DNA, 390 bp, Sitophilus zeamais
catatgatgacccgcaccatgctgtttctggcgtgcgtggcggcgctgtatgtgtgcattagcgcgaccgcgggcaaaccggaagaatttgcgaaactgagcgatgaagcgccgagcaacgatcaggcgatgtatgaaagcattcagcgctatcgccgctttgtggatggcaaccgctataacggcggccagcagcagcagcagcagccgaaacagtgggaagtgcgcccggatctgagccgcgatcagcgcggcaacaccaaagcgcaggtggaaattaacaaaaaaggcgataaccatgatattaacgcgggctggggcaaaaacattaacggcccggatagccataaagatacctggcatgtgggcggcagcgtgcgctggctcgag

SEQ ID NO: 224 - DNA, 34 bp, Artificial Sequence
  ColA Forward primer
gtatctattcccgtctacgaacatatggaattcc

SEQ ID NO: 225 - DNA, 29 bp, Artificial Sequence
  ColA Reverse primer
ccgctcgagccatctgacacttcctccaa

SEQ ID NO: 226 - DNA, 20 bp, Artificial Sequence
  Bacillus forward primer
gaggtagacgaagcgacctg

SEQ ID NO: 227 - DNA, 20 bp, Artificial Sequence
  Bacillus reverse primer
ttccctcacggtactggttc

SEQ ID NO: 228 - DNA, 21 bp, Artificial Sequence
  Buch_groES_18F
catgatcgtgtgcttgttaag

SEQ ID NO: 229 - DNA, 21 bp, Artificial Sequence
  Buch_groES_98R
ctgttcctcgagtcgatttcc

SEQ ID NO: 230 - DNA, 21 bp, Artificial Sequence
  ApEF1a 107F
ctgattgtgccgtgcttattg

SEQ ID NO: 231 - DNA, 21 bp, Artificial Sequence
  ApEF1a 246R
tatggtggttcagtagagtcc

SEQ ID NO: 232 - protein, 13 aa, Pandinum imperator
FLSTIWNGIKGLL

SEQ ID NO: 233 - protein, 13 aa, Urodacus yaschenkoi
ILSAIWSGIKSLF

SEQ ID NO: 234 - protein, 13 aa, Scorpiops tibetanus
LWGKLWEGVKSLI

SEQ ID NO: 235 - protein, 22 aa, Apostichopus japonicus
FPFLKLSLKIPKSAIKSAIKRL

SEQ ID NO: 236 - protein, 13 aa, Urodacus yaschenkoi
ILSAIWSGIKGLL

SEQ ID NO: 237 - protein, 27 aa, Artificial Sequence
  Uy192 + cell penetrating peptide
YGRKKRRQRRRFLSTIWNGIKGLLFAM

SEQ ID NO: 238 - DNA, 20 bp, Sod For
atagctgtccagacgcttcg

SEQ ID NO: 239 - DNA, 20 bp, Sod rev
atgtcgtcgaggcgattacc

SEQ ID NO: 240 - DNA, 20 bp, SACT144 For
ggtgttggcgtacaagtcct

SEQ ID NO: 241 - DNA, 20 bp, SACT314 Rev
gaattgcctgatggacaggt